(19) United States
(12) Patent Application Publication
(10) Pub. No.: US 2025/0144220 A1
(43) Pub. Date: May 8, 2025

(54) CONJUGATE FOR PREVENTING AND TREATING VIRAL INFECTIONS AND USE THEREOF

(71) Applicant: SUZHOU ARK BIOPHARMACEUTICAL CO., LTD., Suzhou (CN)

(72) Inventors: Cheng PENG, Suzhou (CN); Guowei SONG, Suzhou (CN); Zhidong SONG, Suzhou (CN); Shuai YUAN, Suzhou (CN); Weikang LING, Suzhou (CN); Jia MENG, Suzhou (CN); Ta SUN, Suzhou (CN); Zhao GAO, Suzhou (CN); Gang ZOU, Suzhou (CN); Haiging YUAN, Suzhou (CN); Zhen Jim WU, Suzhou (CN)

(21) Appl. No.: 18/719,549

(22) PCT Filed: Dec. 29, 2022
(86) PCT No.: PCT/CN2022/143387

§ 371 Date: Jun. 13, 2024

(30) Foreign Application Priority Data
Dec. 30, 2021 (CN) 202111653630.5

(51) Int. Cl. A61K 47/54; A61K 31/13; A61K 31/196; A61K 31/215; A61K 31/351; A61K 31/506; A61K 31/5383; A61K 39/42; A61P 31/16; C07D 309/28; C07D 405/14

(52) U.S. Cl. CPC A61K 47/545; A61K 31/13; A61K 31/196; A61K 31/215; A61K 31/351; A61K 31/506; A61K 31/5383; A61K 39/42; A61K 47/543; A61P 31/16; C07D 309/28; C07D 405/14

(57) ABSTRACT

The present invention relates to a conjugate for preventing and treating viral infections and a use thereof, and in particular, to a protein-anti-influenza compound conjugate having a structure of formula I-1 or a pharmaceutically acceptable salt, an ester, an isomer, a solvate, a prodrug or an isotope label thereof. The conjugate has small molecules (D¹ and D²) linked, by means of linkers (L¹ and L²) to an Fc monomer, an Fc domain, an Fc linker peptide, an albumin or an albumin linker peptide (E) and having anti-influenza virus activity. The conjugate or the intermediate compound of the present invention has significant anti-influenza virus activity, and meanwhile, has excellent in vitro/in vivo pharmacokinetic property and safety and has good clinical application prospects.

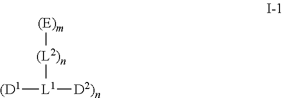

### TECHNICAL FIELD

The present disclosure relates to the conjugates for anti-influenza (Flu) virus drugs and antibodies or its constant region Fc. Specifically, it relates to the compound containing antibodies or its constant region Fc that is covalently attached to small molecular inhibitors of viral surface protein or inhibitors of peptide drugs, and the intermediates thereof, and relates to the pharmaceutical composition or drug comprising the same, and to their use in the prevention and/or treatment of related influenza viral infections.

### BACKGROUND

The influenza virus (Influenza virus) is responsible for nearly three to five million cases of severe infections annually and around 500,000 deaths worldwide (Luliano et al., 2018, Lancet 391: 1285-1300). While most healthy people recover from the virus infection on their own within one to two weeks, influenza virus infections can develop into life-threatening infections and complications, such as pneumonia, in older adults, people with chronic illnesses, and those with a weak immune system.

It is an ongoing faced challenge for human to develop treatments for influenza viruses. While there are already anti-influenza virus drugs and prophylactic vaccines approved on the market, anti-influenza small molecule drugs typically need to be taken within 48 hours of the onset of infection symptoms in order to have a clinical benefit. In addition, due to the high variability of the influenza virus, drug-resistant virus strains have been found to emerg against these commonly used drugs. Therefore, there is a need to develop a more effective and long-lasting therapy for the treatment and/or prevention of influenza viruses.

Influenza virus is a kind of negative-stranded, segmented RNA virus belonging to the family Orthomyxoviridae, which includes the genus Influenza virus A, B, and C. In humans, influenza A and B virus infections predominate. Influenza viruses infect respiratory epithelial cells. The first step in the process is the adsorption process to the host cell mediated by the virus surface receptor-binding protein (the hemagglutinin protein, HA protein, for influenza proteins). Subsequently, in the presence of the hemagglutinin protein, the viral envelope fuses with the cell membrane, and the influenza virus genome fragment and the viral RNA-dependent RNA polymerase complex are subsequently released into the cell. The viral RNA-dependent RNA polymerase complex uses the genomic fragment as a template to synthesize new progeny virus particles inside the cell. The newly synthesized virus particles are released outside the cell via cell lysis or budding. For influenza virus, complete release of the progeny virus depends on the cleavage of sialic acid residues on cell surface by neuraminidase NA. The Neuraminidase inhibitors that target the neuraminidase of influenza viruses to reduce viral spread have been approved in clinical trials, including oseltamivir (Tamiflu™), zanamivir (Relenza™) and peramivir (Rapivab™).

Patients who have received organ transplants or cancer patients are not able to effectively clear the virus due to a suppressed immune system, leading to the prolonged in vivo virus replication after infection of influenza viruses, and thus the increased occurrence of the drug-resistant strains as has already been found in the clinical. Therefore, there is a need for the new and more effective influenza treatments and therapeutic agents.

### SUMMARY

In one aspect, the present disclosure provides a conjugate of formula I-1 that includes an anti-influenza virus small molecule coupled to a protein (also referred to as the “protein-anti-influenza compound conjugate”) has a significant anti-influenza virus biological activity.

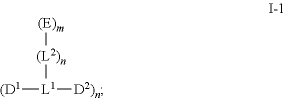

Specifically, the protein-anti-influenza compound conjugates in present disclosure have small molecules (D¹ and D²) having anti-influenza viral activity and linked to an antibody or an antibody fragment thereof, an Fc monomer, an Fc structural domain, an Fc-linked peptide, an albumin or an albumin-linked peptide (E) via a linker (L¹ and L²). The protein-anti-influenza compound conjugates in present disclosure have a significant anti-influenza virus activity, as well as the excellent in vitro/in vivo pharmacokinetic property and safety, the long half-life, resulting in a good application prospect in clinical.

The Embodiments described as below are provided in present disclosure:

Present disclosure provides a conjugate of formula I-1, or a pharmaceutically acceptable salt, ester, isomer, solvate, prodrug or isotopically labeled derivative thereof,

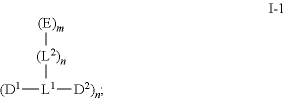

wherein,
m is 1 or 2;
n is an integer from 1 to 20, preferably an integer from 1 to 10, e.g., 1, 2, 3, 4, 5, 6, 7, 8, 9 or 10; and two n's are identical;
each of D1 and D2 is an anti-influenza virus small molecule drug, and is independently selected from a compound having an anti-influenza activity as shown in formulae D-1-1 to D-1-8:

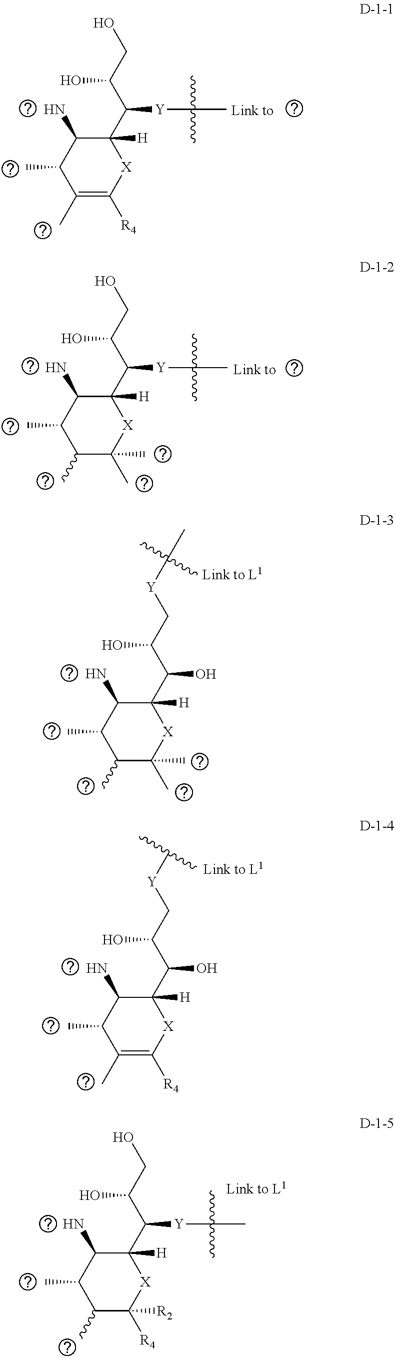

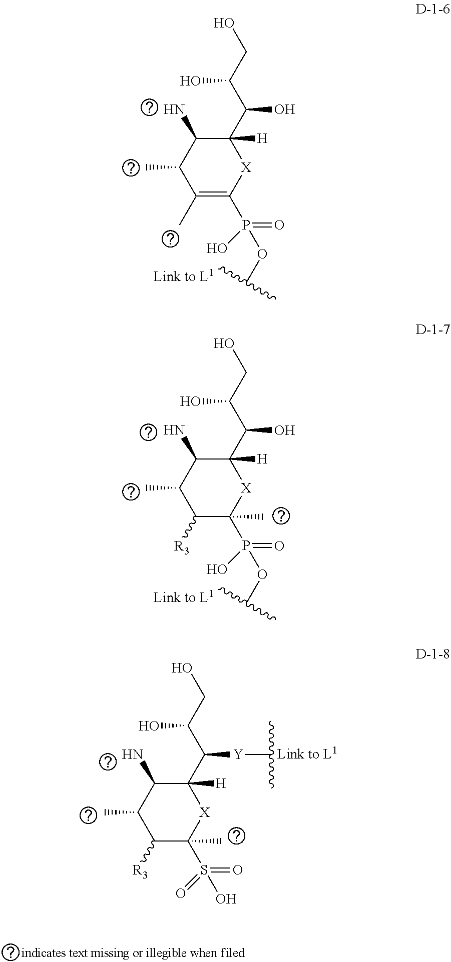

R₁ is selected from OH, NH₂, —NH(═NH)NH₂ and —NHC(═NH)NHR₆; R₂ and R₃ are each independently selected from H, OH, F, Cl and Br; R₄ is selected from —COOH, —P(═O)(OH)₂ and —SO₃H;
R₅ is selected from —COC₁₋₆ alkyl, —COC₁₋₆ haloalkyl, —SO₂C₁₋₆ alkyl, and —SO₂C₁₋₆ haloalkyl; preferably selected from —COCH₃, —COCF₃, and —SO₂CH₃;
X is selected from O and S;
R⁶ is selected from the following groups:

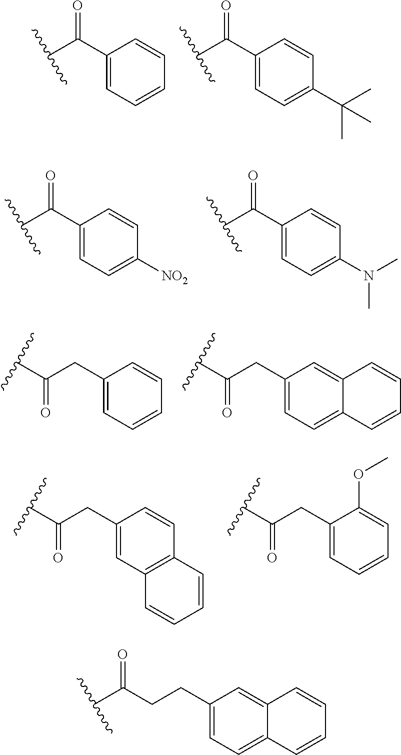

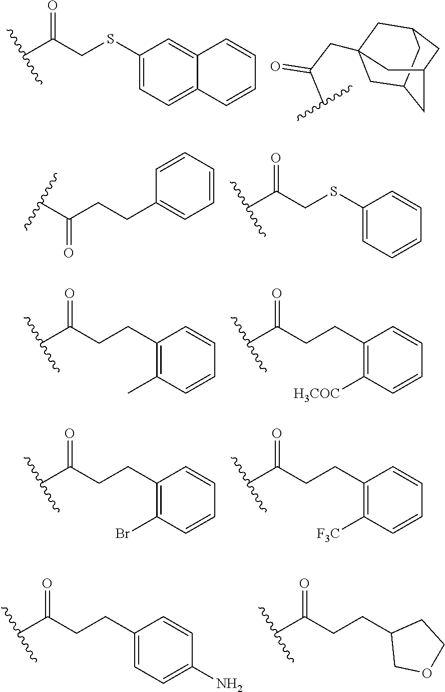

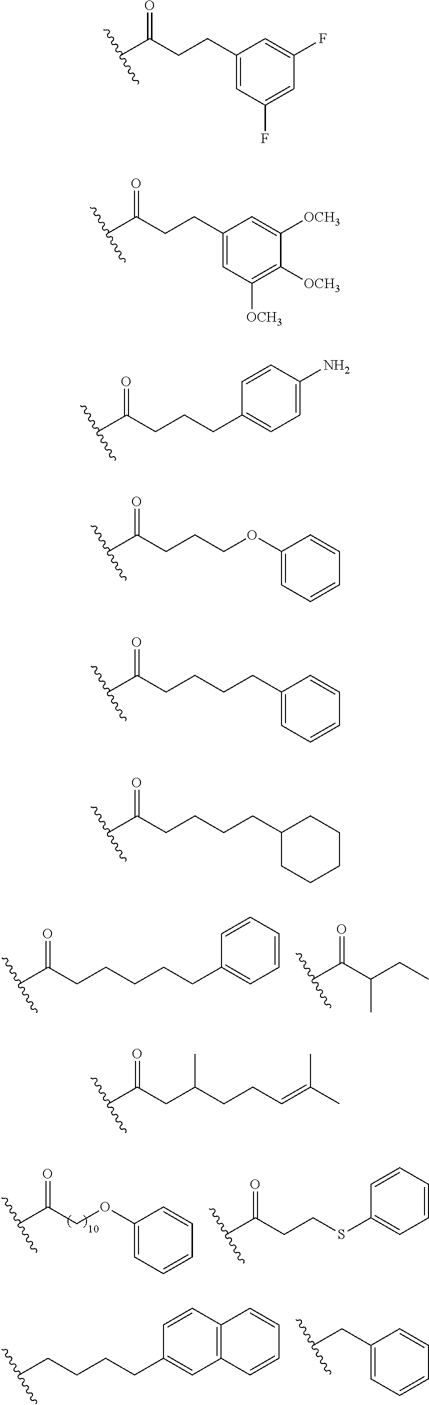

Y is selected from the following groups, wherein “(attached to L¹)” specifies the end of the group Y attached to the end of L, wherein N denotes an atom within the ring when drawn inside the ring:

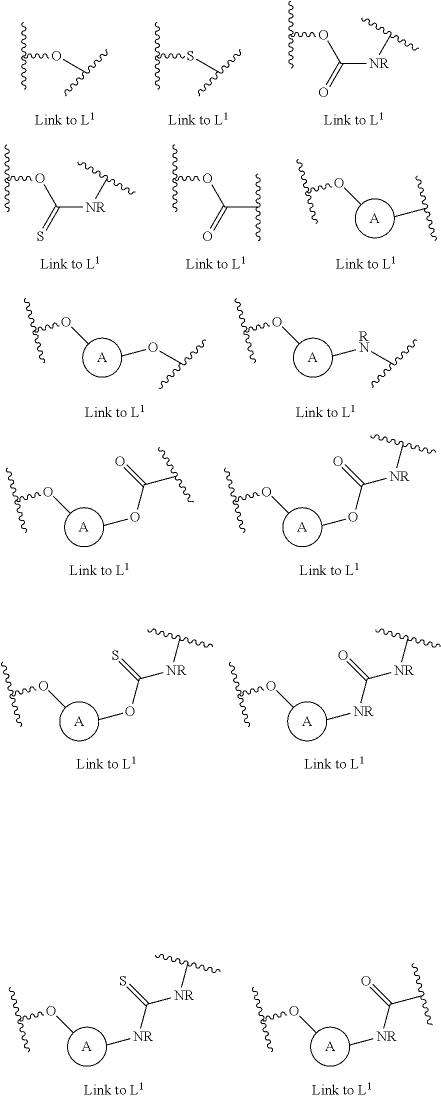

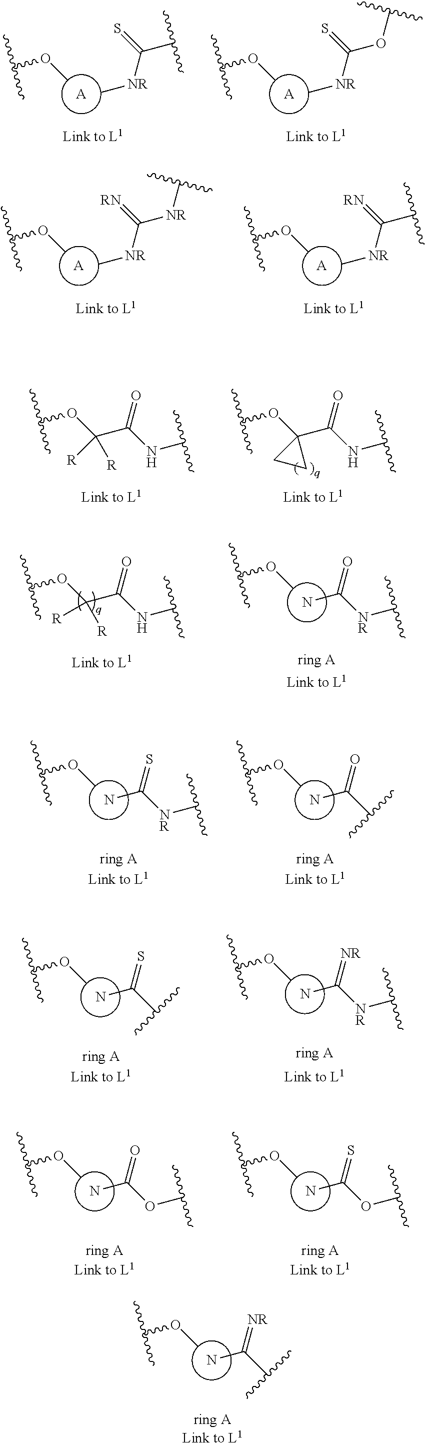

ring A is selected from C₃-C₂₀ cycloalkyl, substituted C₃-C₂₀ cycloalkyl, C₃-C₂₀ cycloalkenyl, substituted C₃-C₂₀ cycloalkenyl, C₆-C₁₅ aryl, 3- to 20-membered heterocycloalkyl, substituted 3- to 20-membered heterocycloalkyl, substituted C₆-C₁₅ aryl and substituted 5- to 15-membered heteroaryl;
R groups are each independently selected from H, deuterium, optionally substituted C₁-C₂₀ alkyl, optionally substituted C₂-C₂₀ streptenyl, optionally substituted C₃-C₂₀ cycloalkyl, optionally substituted 3- to 20-membered heterocycloalkyl, optionally substituted C₆-C₁₅ aryl, and optionally substituted 5- to 15-membered heteroaryl;
q is 0, 1, 2, 3, 4, 5, 6, 7, 8, 9 or 10;
L¹ is a linker attached to the drug (D¹ or D²) and L², and includes but not limited to following structures.

It should be understood that, bonds at left and right ends of the following structures are attached to an oxygen atom of the drug and a bond marked by the wavy line in the middle is attached to L²,

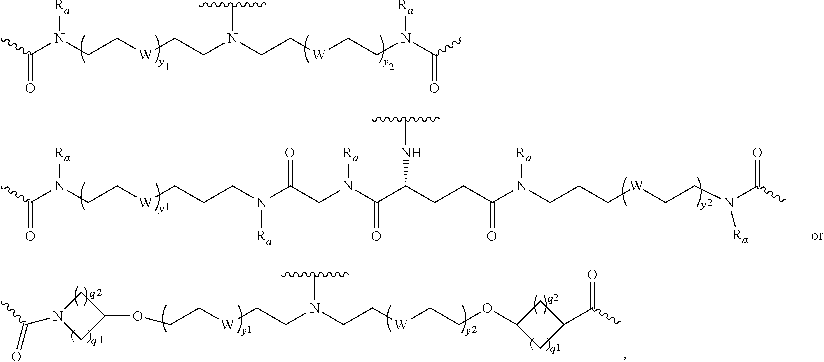

wherein:
W is selected from O, S, NR^{b}, CH₂ or absent;
R^{a} and R^{b} are each independently selected from H, optionally substituted C₁-C₂₀ alkyl, optionally substituted C₂-C₂₀ alkenyl;
y¹ and y² are each independently 0, 1, 2, 3, 4, 5 or 6;
q¹ and q² are each independently selected from 1, 2, 3, 4, 5 and 6;
or
L¹ is selected from following structures:

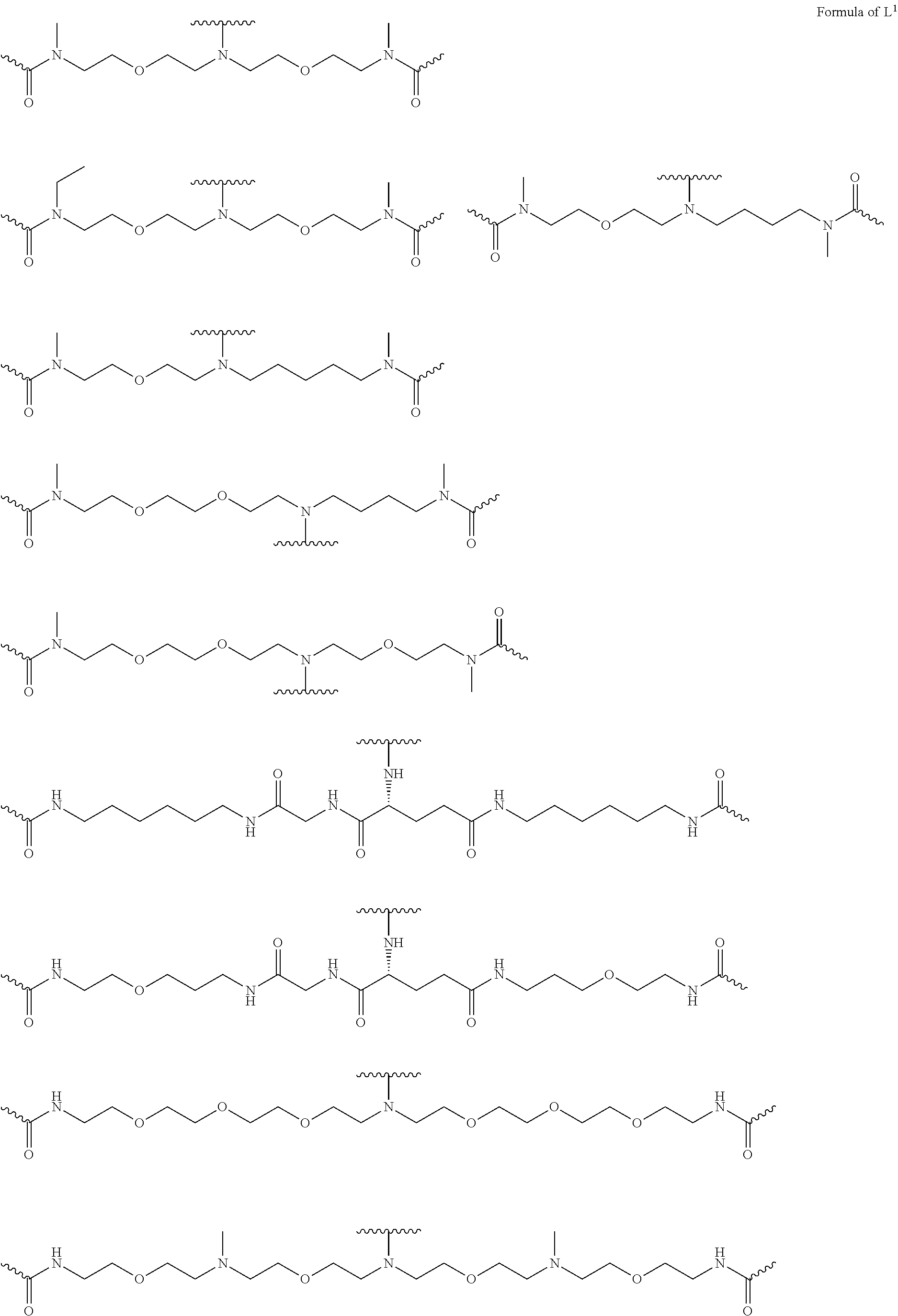

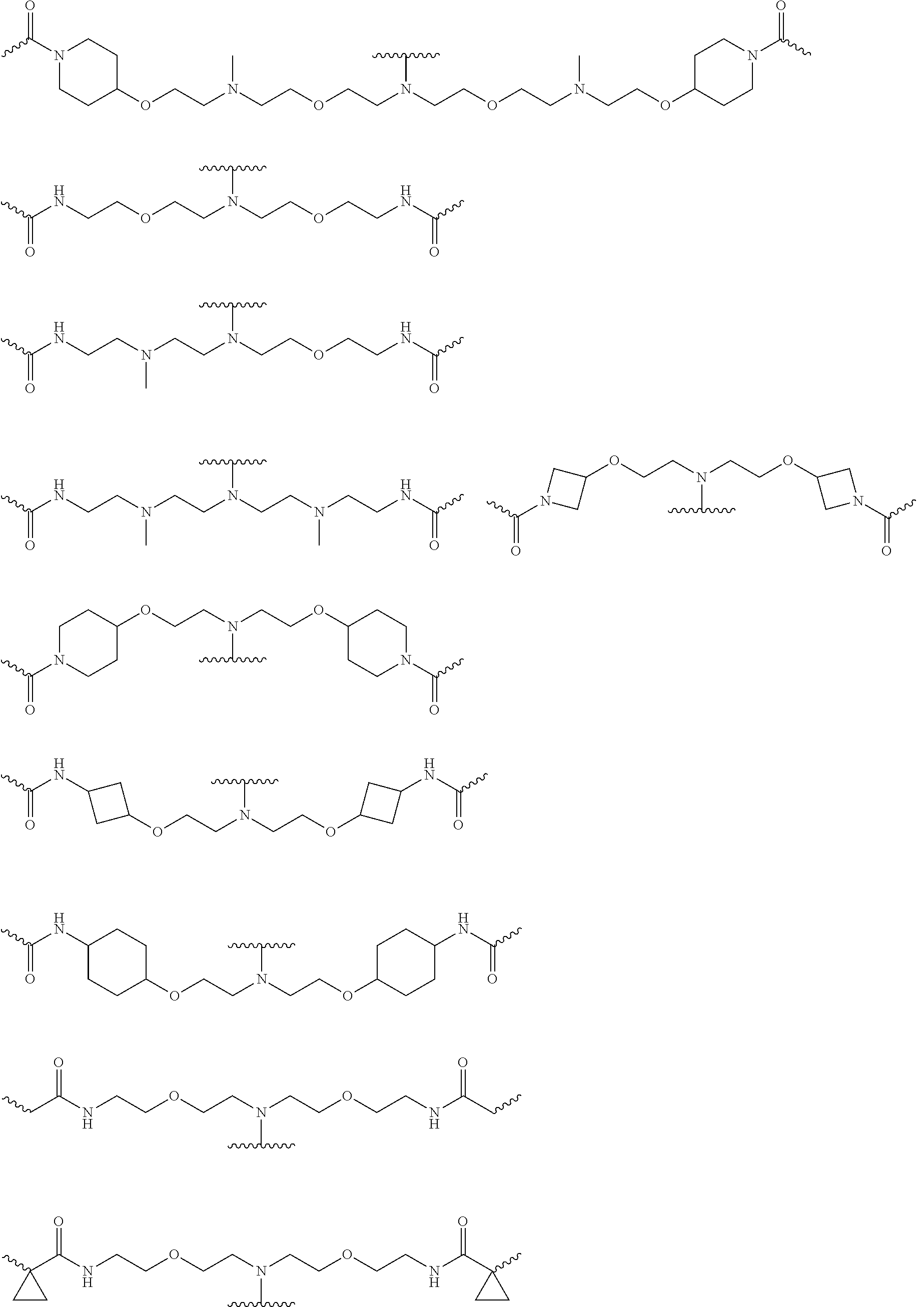

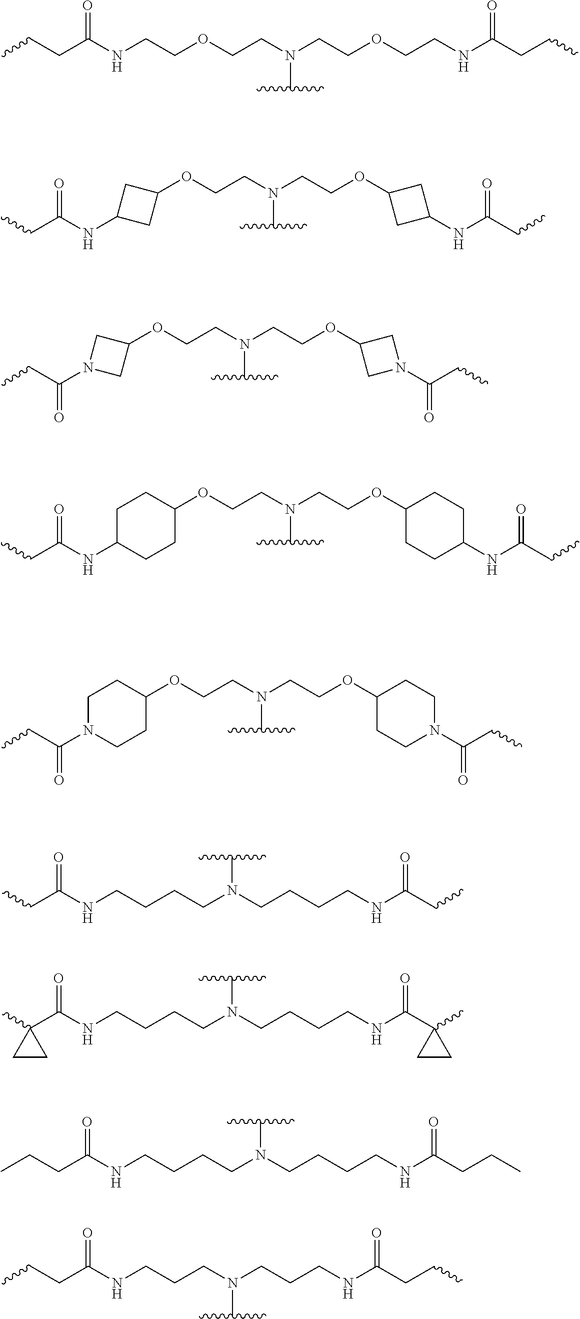

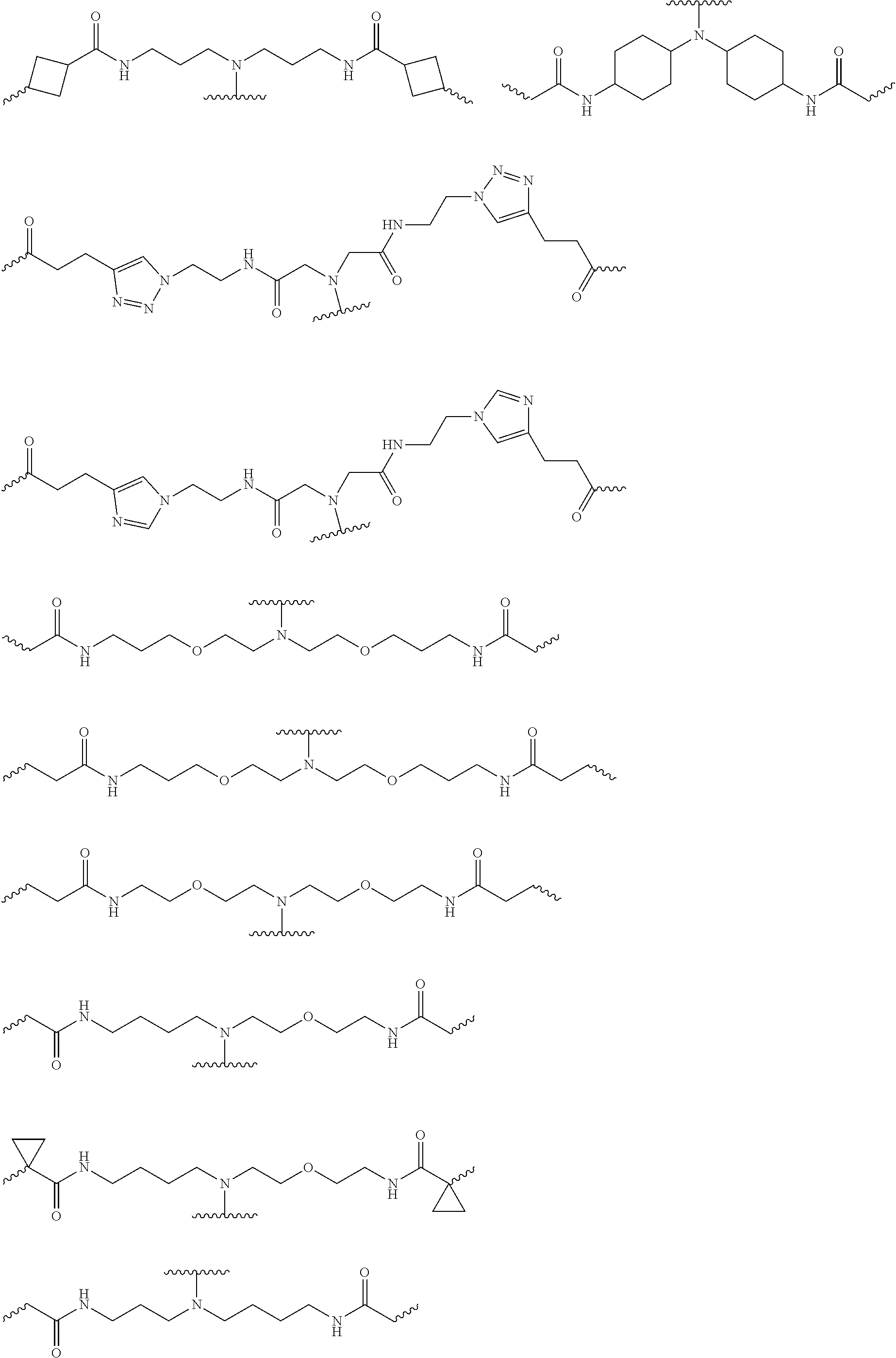

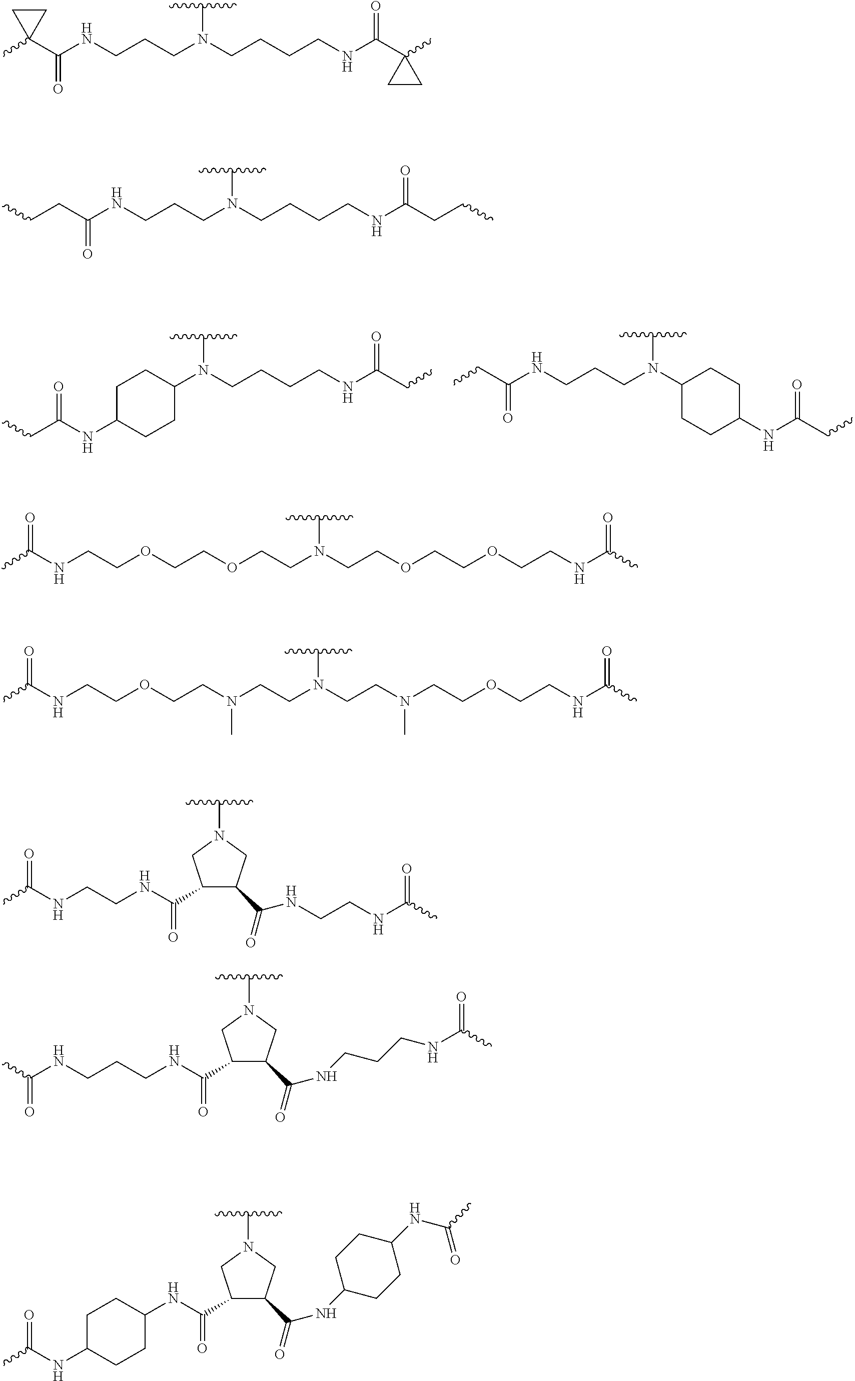

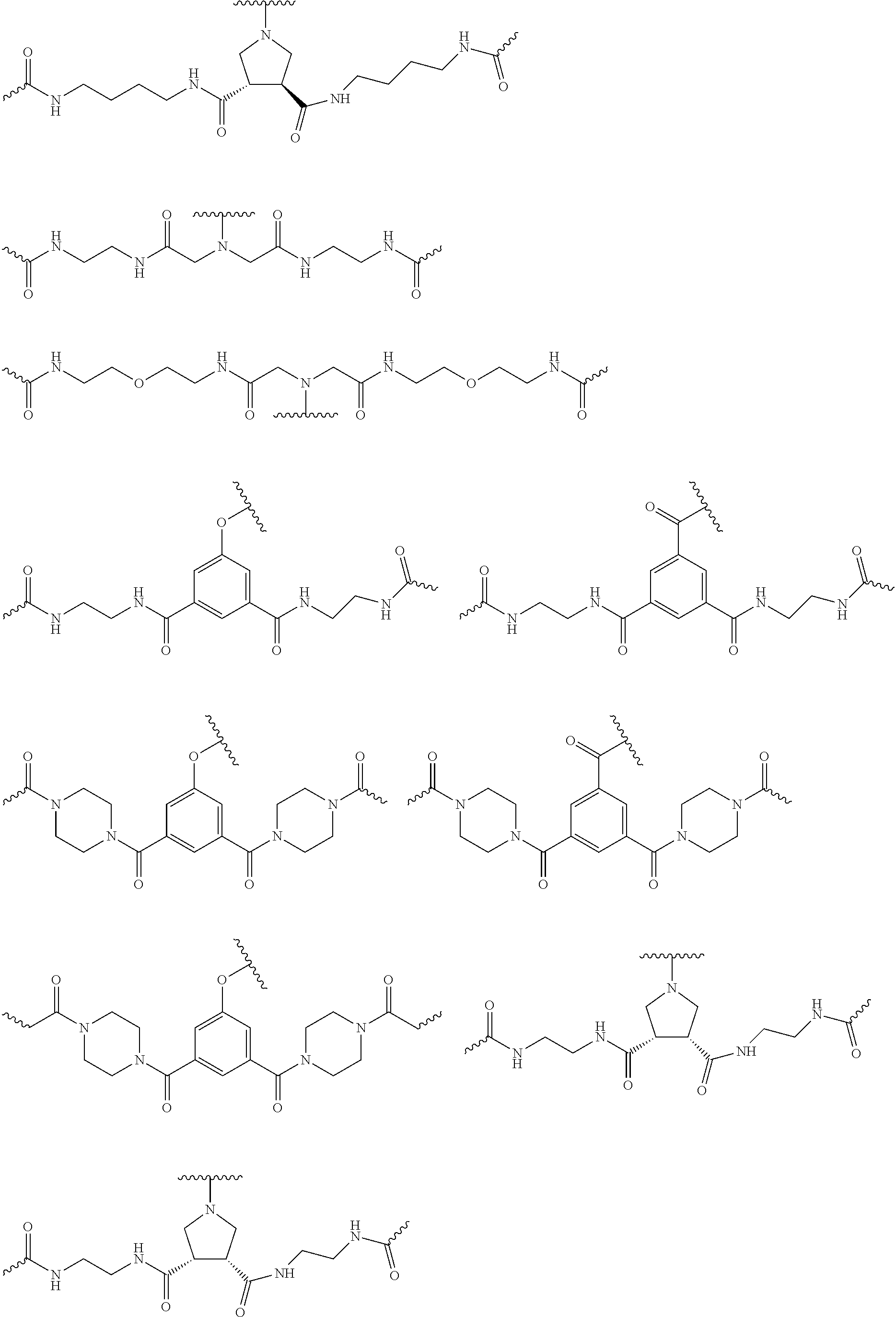

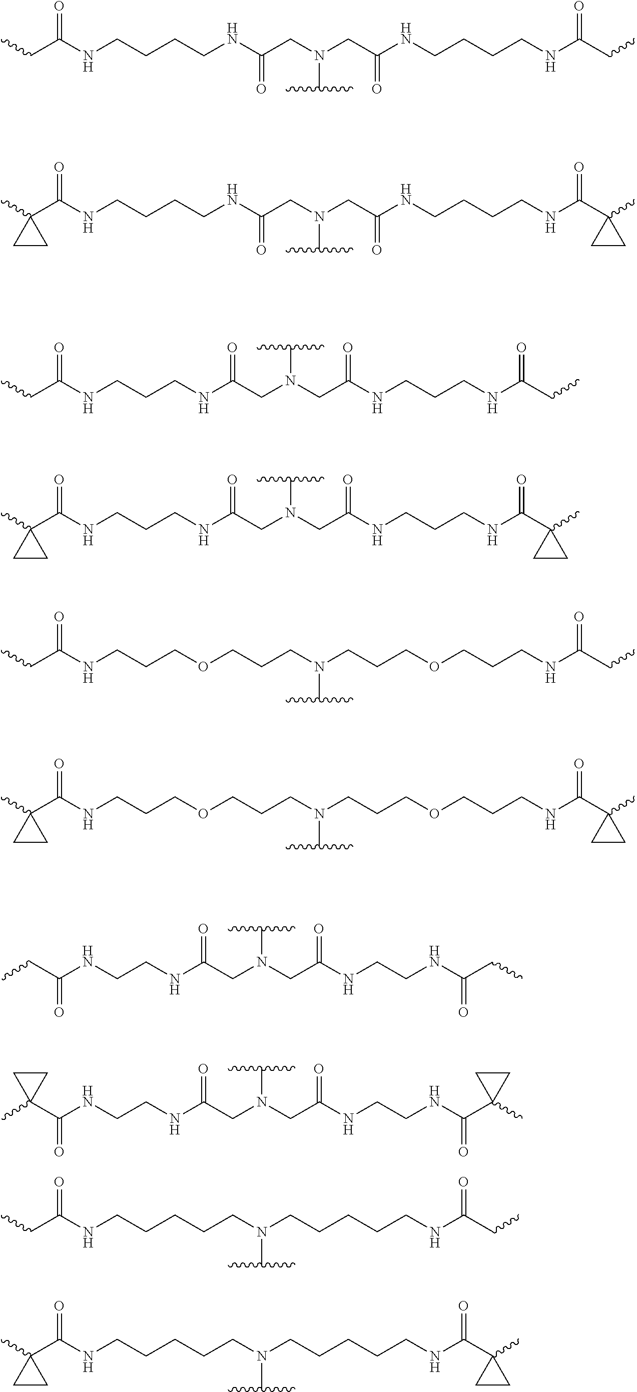

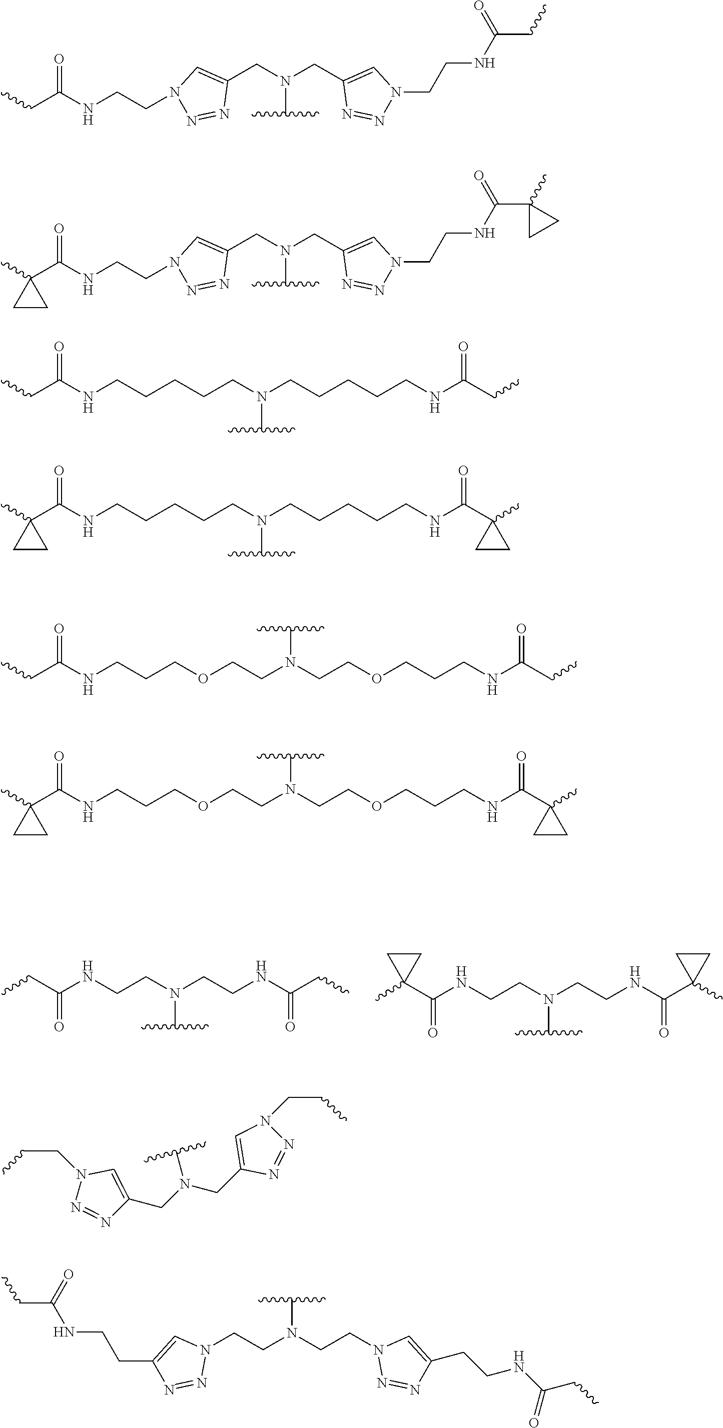

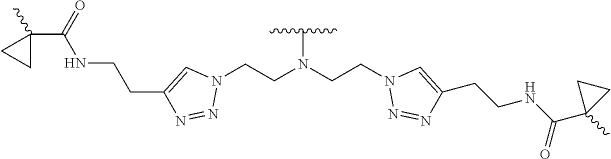

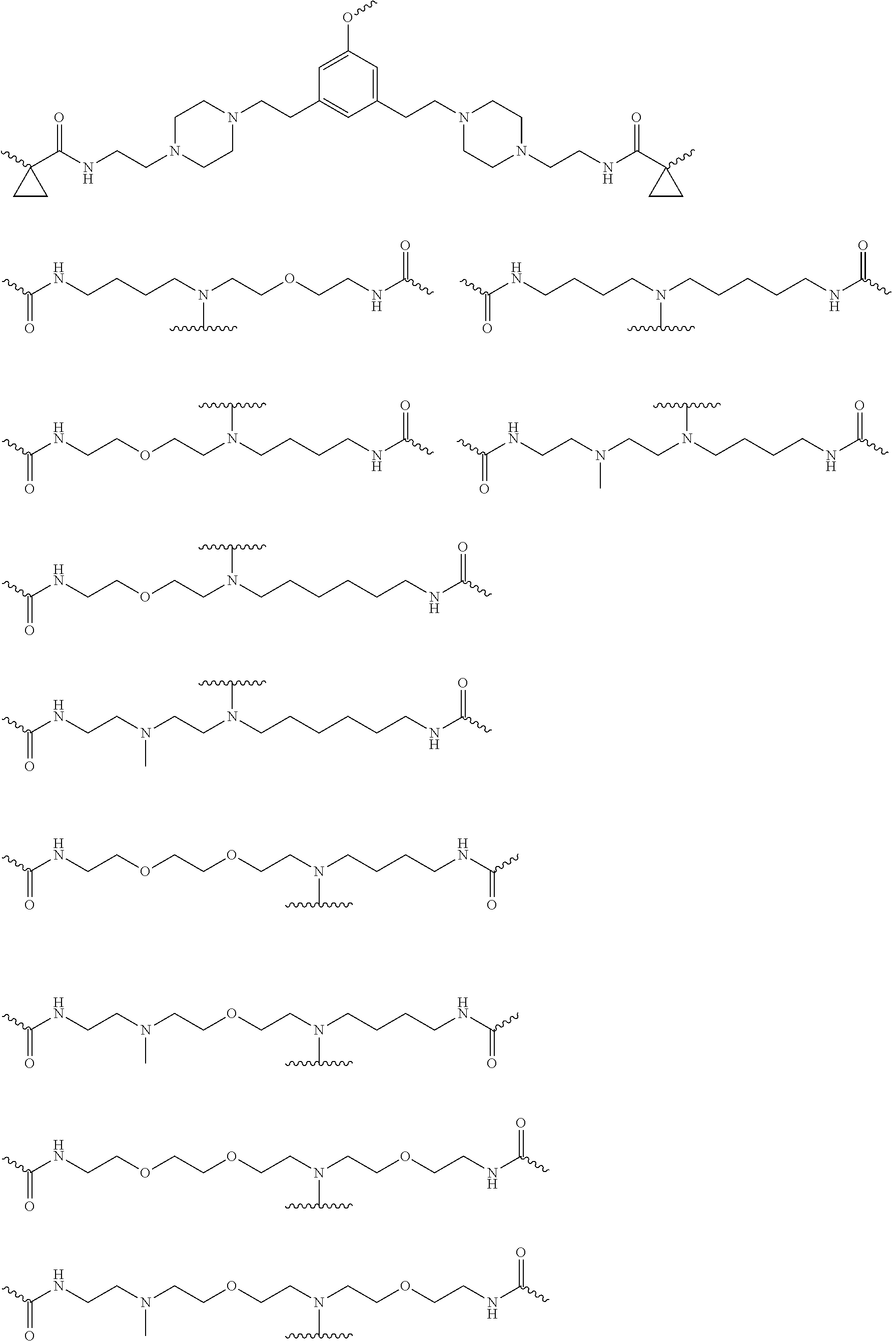

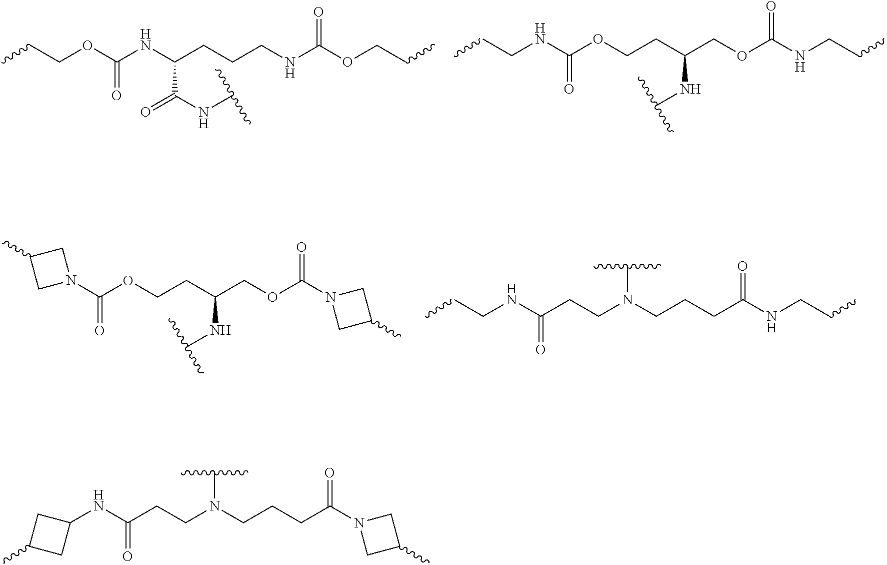

L² is a linker attaching L¹ and E (Fc monomer, Fc structural domain, Fc-binding peptide, albumin, or albumin-linking peptide) and having a structure sleeted from formula L2-1 to L2-15, wherein (L¹) and (E) denote ends of L² connected to L¹ and E, respectively:

| L² structure No. | Structure |
|---|---|
| L2-1 | 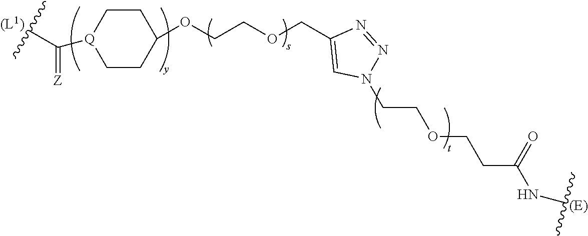 |
| L2-2 | 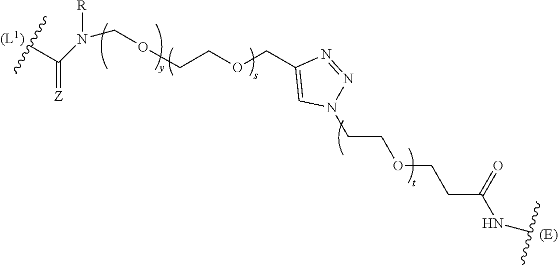 |
| L2-3 | 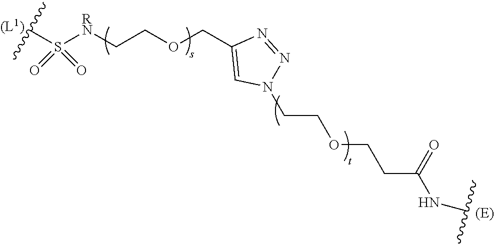 |
| L2-4 | 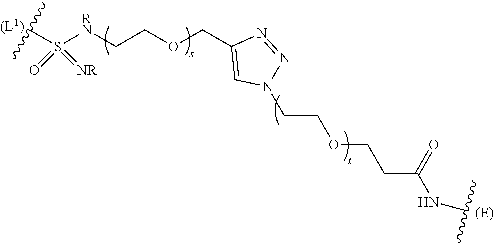 |
| L2-5 | 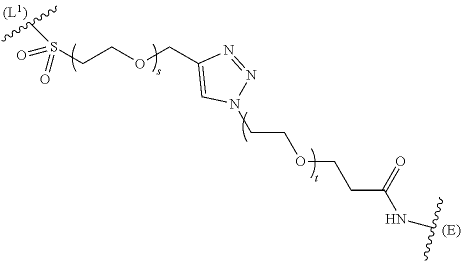 |
| L2-6 | 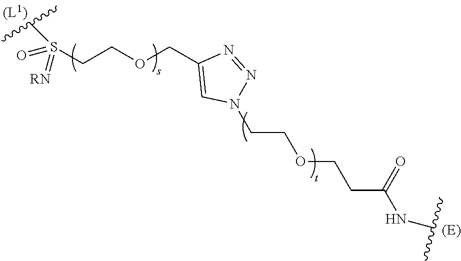 |
| L2-7 | 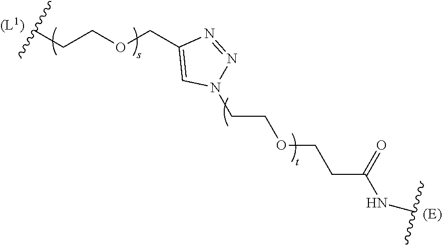 |
| L2-8 | 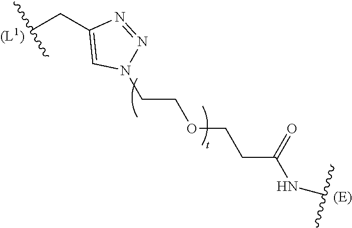 |
| L2-9 | 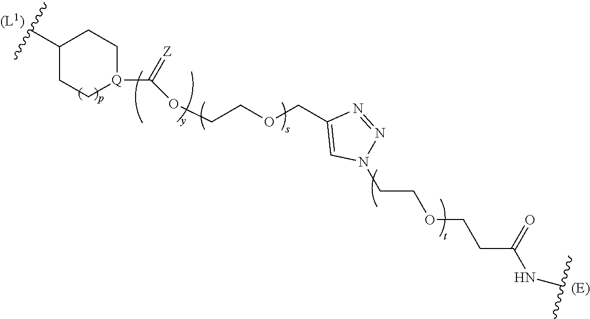 |
| L2-10 | 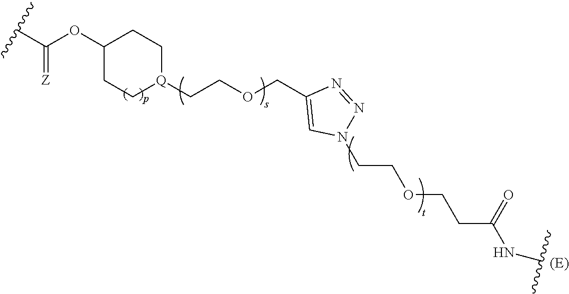 |
| L2-11 | 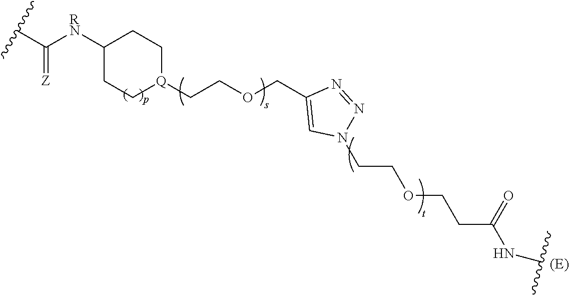 |
| L2-12 | 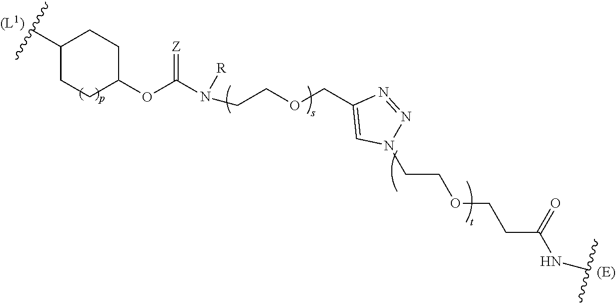 |
| L2-13 | 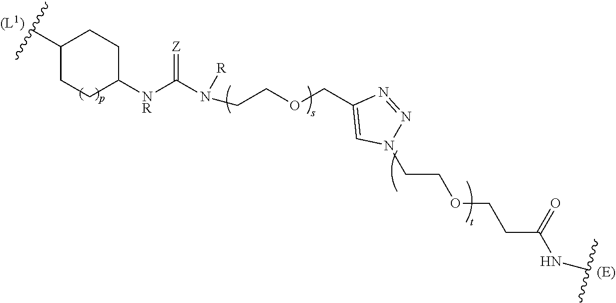 |
| L2-14 | 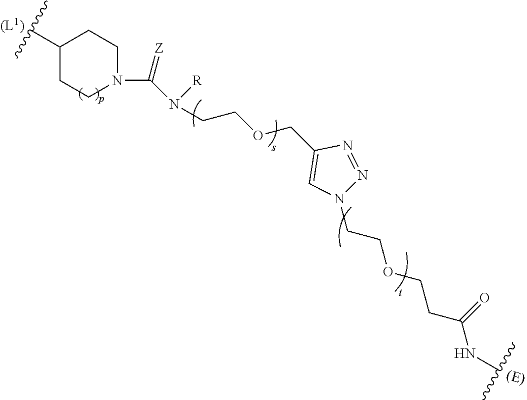 |
| L2-15 | 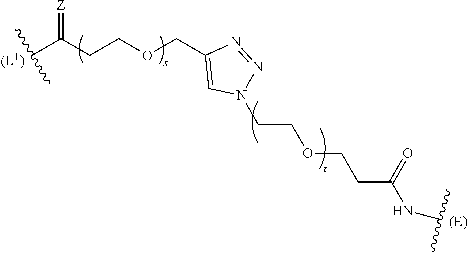 |

wherein,
Z is NR, S or O;
R are each independently selected from hydrogen, deuterium, optionally substituted C₁-C₂₀ alkyl, optionally substituted C₂-C₂₀ chain alkenyl, optionally substituted C₃-C₂₀ cycloalkyl, optionally substituted 3- to 20-membered heterocycloalkyl, optionally substituted C₆-C₁₅ aryl and optionally substituted 5- to 15-membered heteroaryl;
s and t are integers from 1 to 20; such as 1, 2, 3, 4, 5, 6, 7, 8, 9, 10, 11 or 12;
y is 0 or 1;
Q is N or CH;
p is 0, 1, 2, 3, 4, 5, 6, 7 or 8;
E is an antibody or an antibody fragment thereof, an Fc structural domain monomer, an Fc structural domain, an Fc-binding peptide, an albumin or an albumin-linking peptide, and comprises an amino acid sequence of any one of SEQ ID Nos. 1-68 or an amino acid sequence that is at least 95% identical to any one of SEQ ID Nos. 1-68. In one embodiment, in the compound of formula I-1, dimerization of the E single structural domain forms the Fc structural domain.

Further provided is a conjugate of formula I-1, or a pharmaceutically acceptable salt, ester, isomer, solvate, prodrug or isotopically labeled derivative thereof.

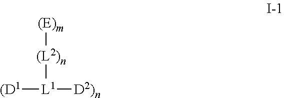

wherein
m is 1 or 2;
n is an integer from 1 to 20, preferably an integer from 1 to 10;
each of D¹ and D² is independently selected from the compound having an anti-influenza activity as shown in the formulae D-1-1 to D-1-8;
D¹ and D² are covalently attached to L¹ that is covalently attached to L², and L² is covalently attached to E;
E is selected from the Fc structural domain monomer, the Fc structural domain, the Fc-binding peptide, the albumin or the albumin-linking peptide as defined above.

In some embodiments, the conjugate of formula I-1 has a structure of formula I-1-1 to I-1-8:

| No. | Structure |
|---|---|
| I- 1-1 | 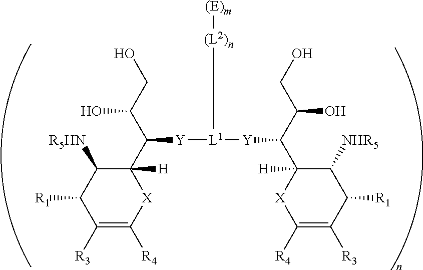 |
| I- 1-2 | 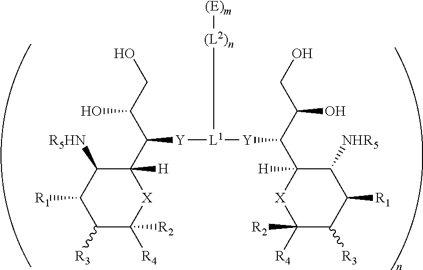 |
| I- 1-3 | 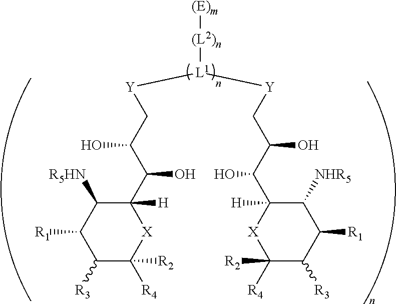 |
| I- 1-4 | 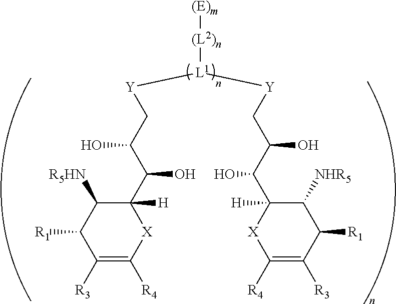 |
| I- 1-5 | 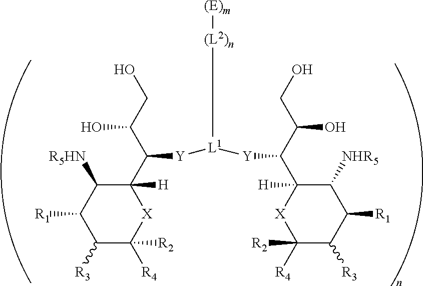 |
| I- 1-6 | 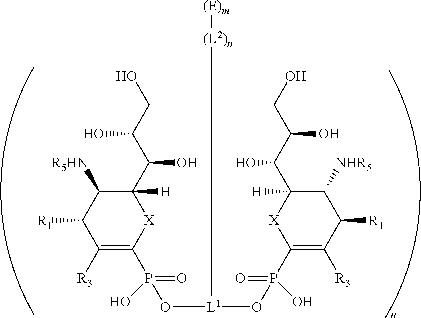 |
| I- 1-7 | 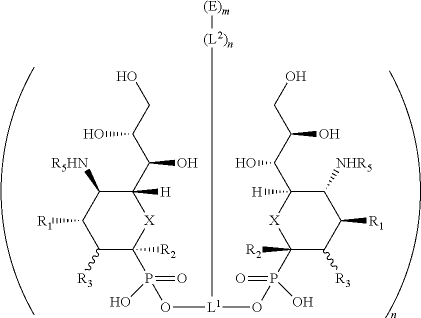 |
| I- 1-8 | 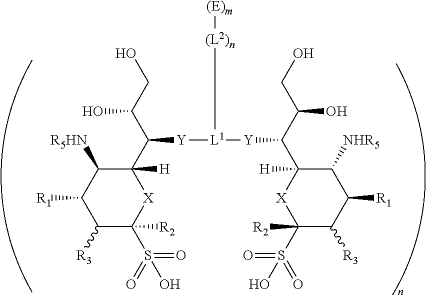 |

wherein each variable (such as R¹-R⁵, X, Y, E, L¹, L², m and n) is as defined above.

In some embodiments, the compounds of formula I-1-1 to I-1-8 are selected from structures of formula I-1-A to I-1-D:

| Compound No. | Structure |
|---|---|
| I-1-A | 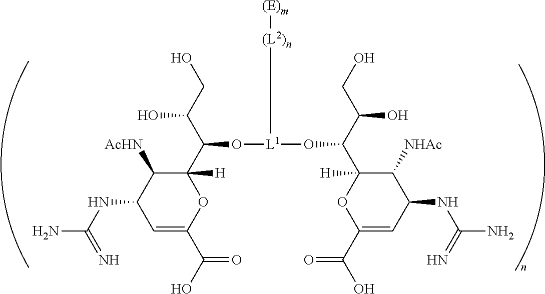 |
| I-1-B | 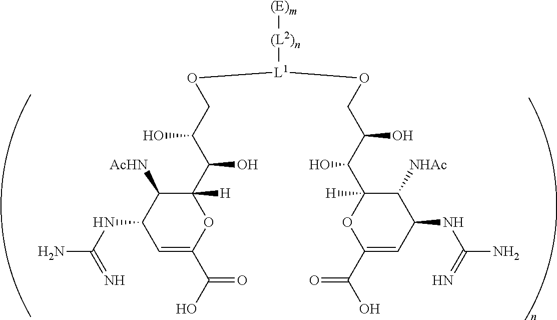 |
| I-1-C | 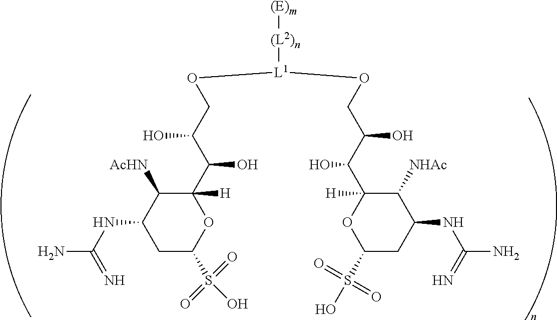 |
| I-1-D | 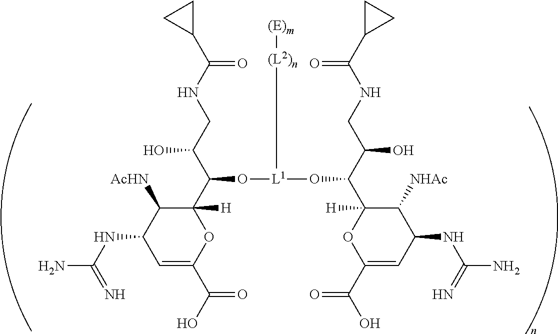 |

wherein each variable (such as E, L¹, L², m and n) is as defined above.

In some embodiments, the conjugate of formula I-1 preferably selected from conjugates of formula C-1 to C-115:

| Con- | |
|---|---|
| jugate | |
| No. | Conjugate Structure |
| C-1 | 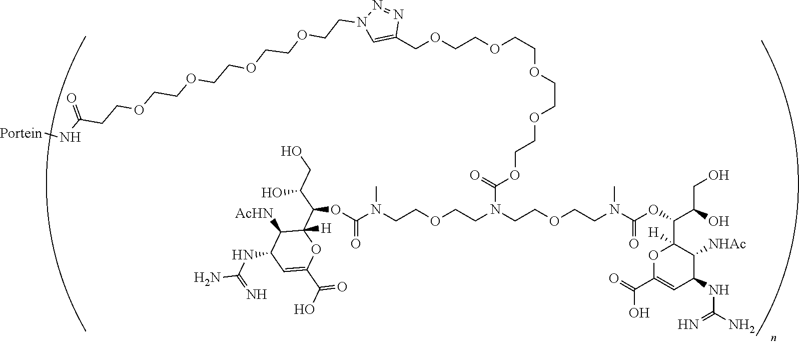 |
| C-2 | 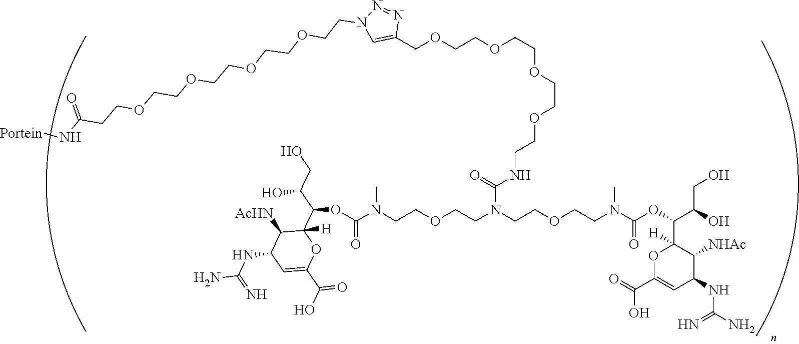 |
| C-3 | 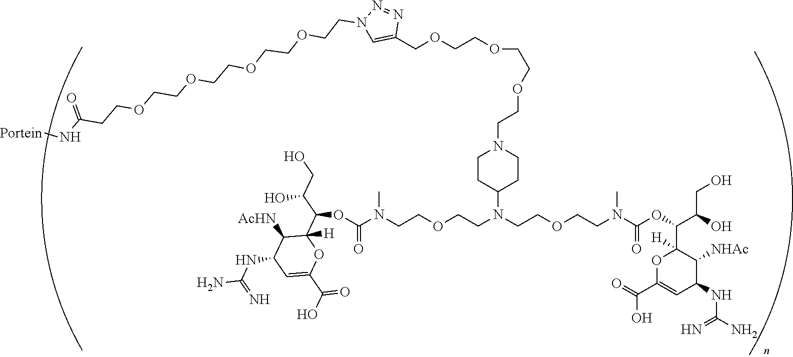 |
| C-4 | 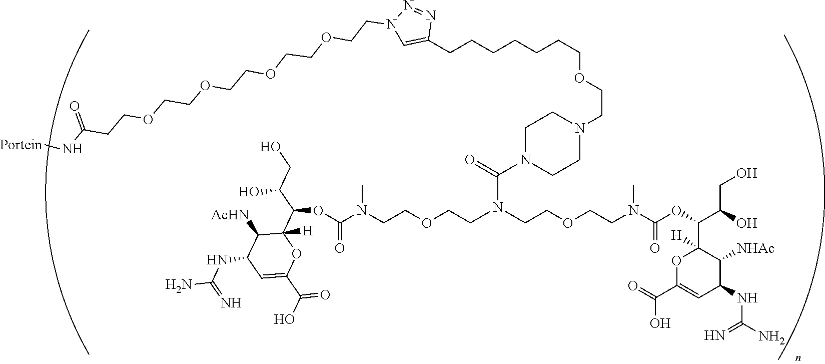 |
| C-5 | 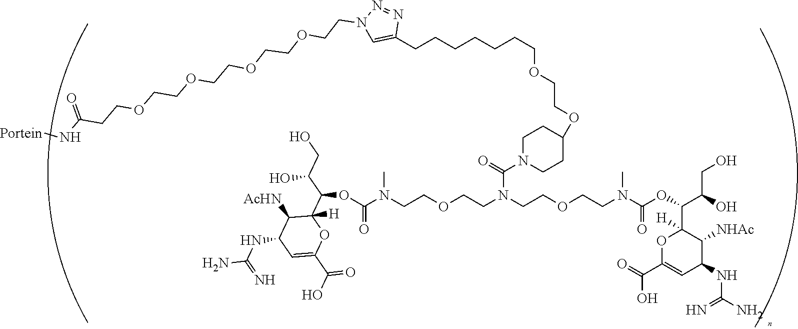 |
| C-6 | 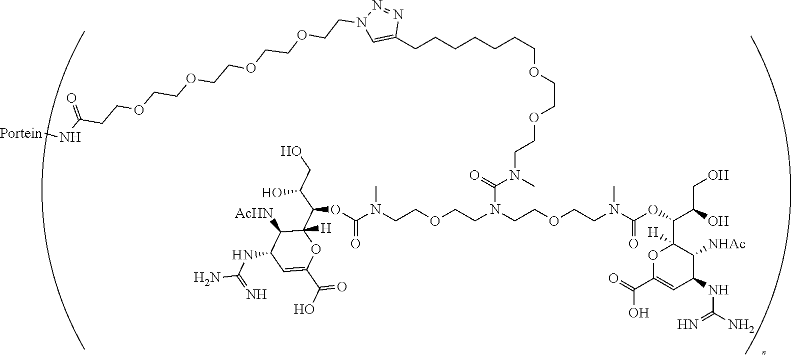 |
| C-7 | 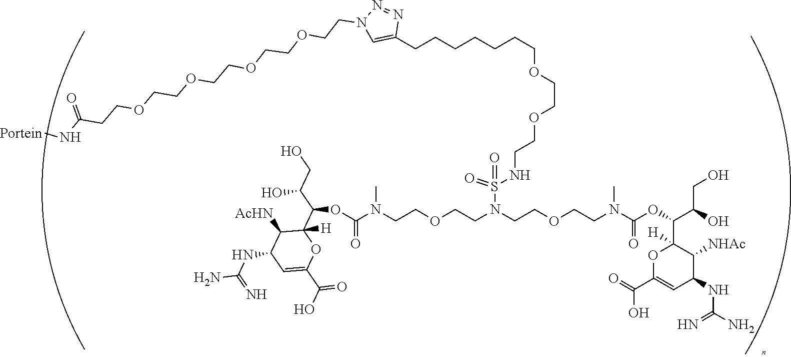 |
| C-8 | 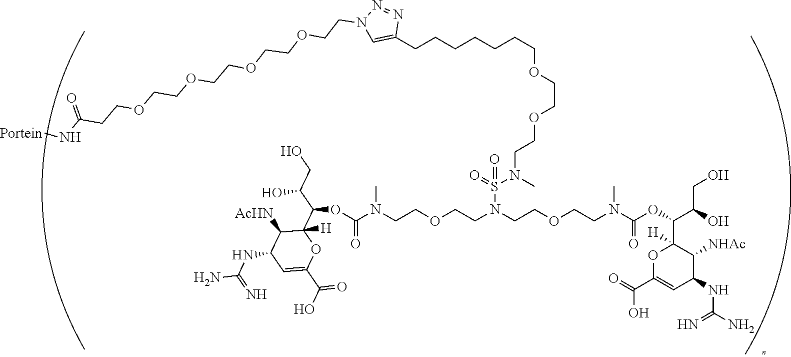 |
| C-9 | 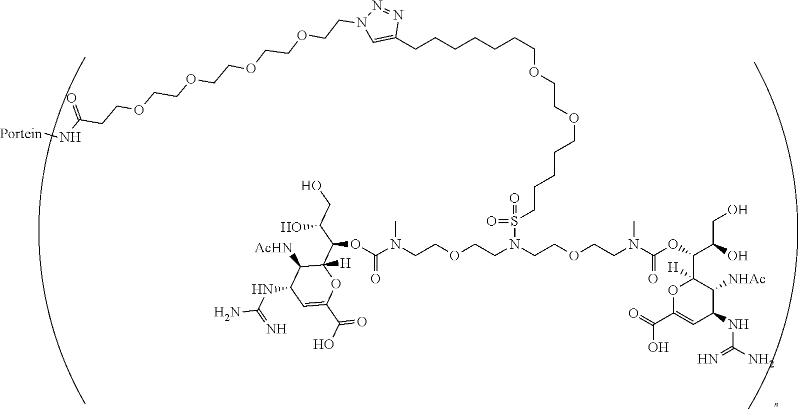 |
| C-10 | 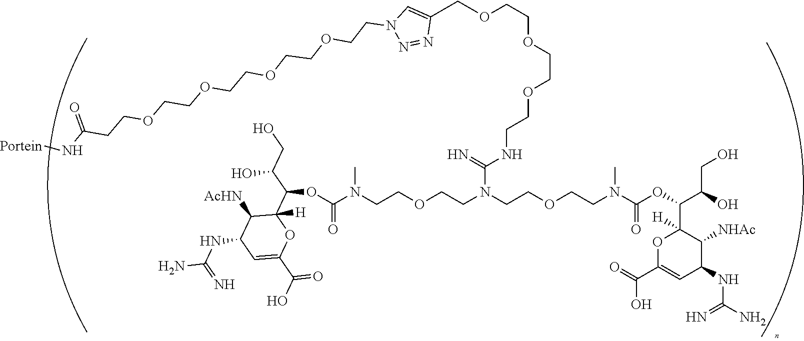 |
| C-11 | 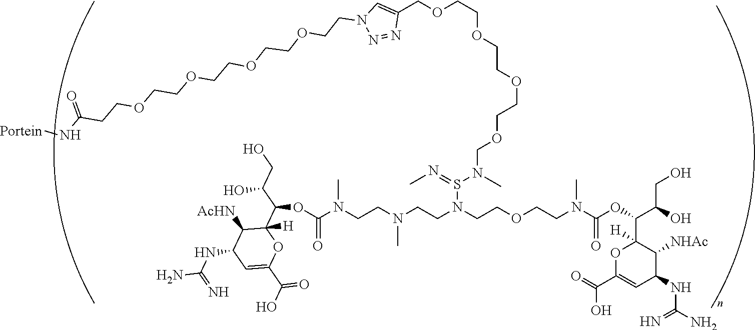 |
| C-12 | 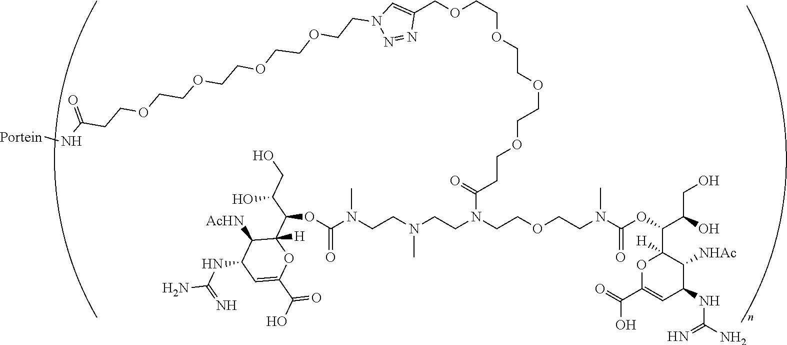 |
| C-13 | 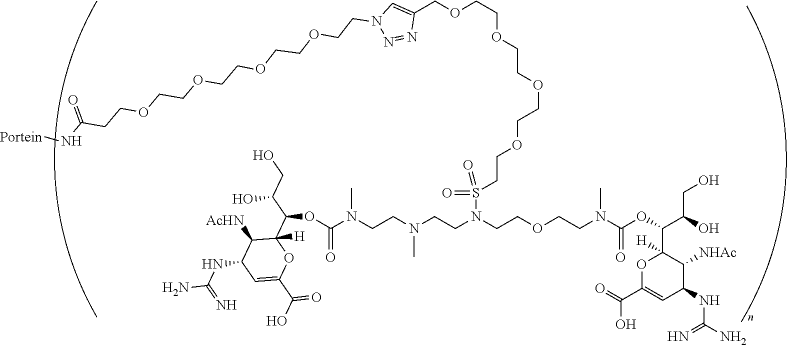 |
| C-14 | 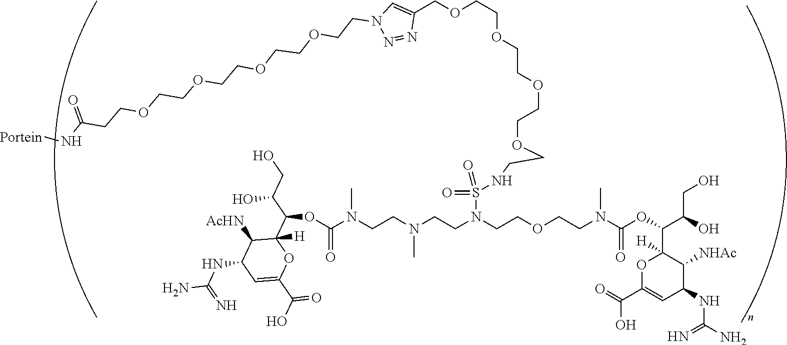 |
| C-15 | 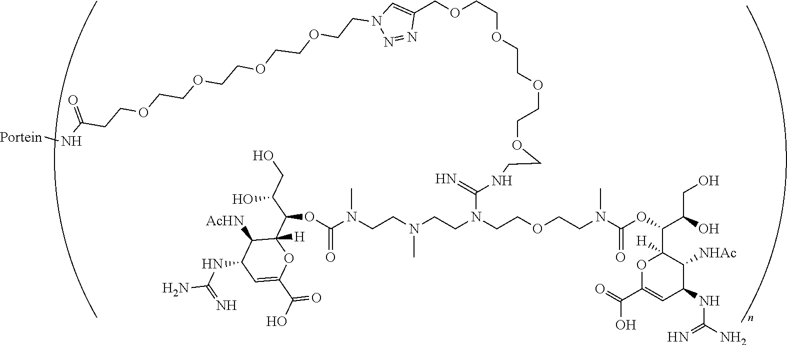 |
| C-16 | 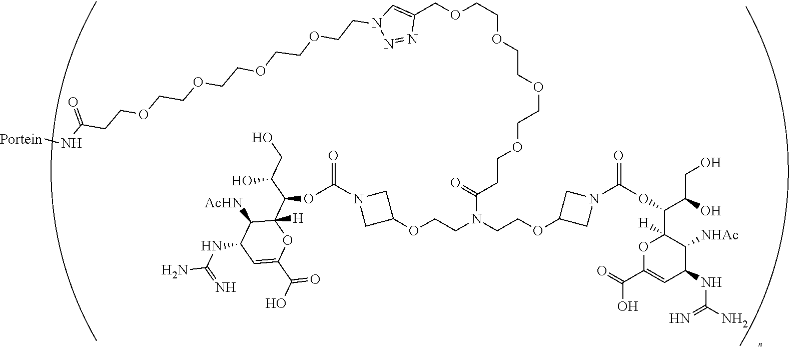 |
| C-17 | 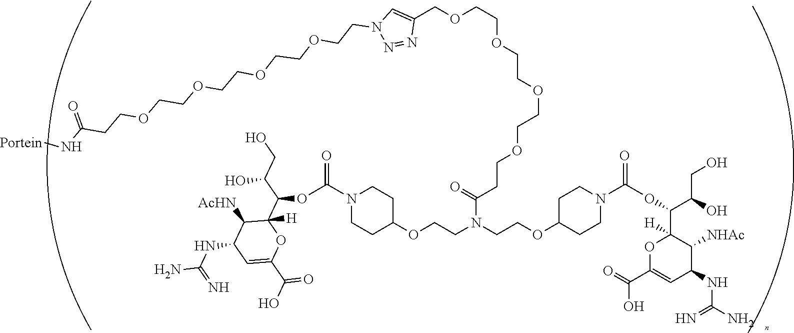 |
| C-18 | 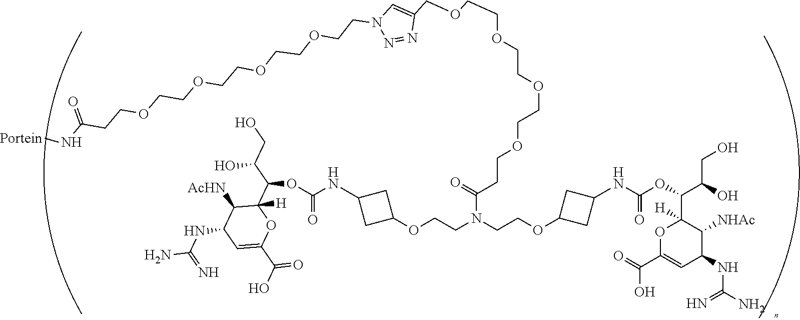 |
| C-19 | 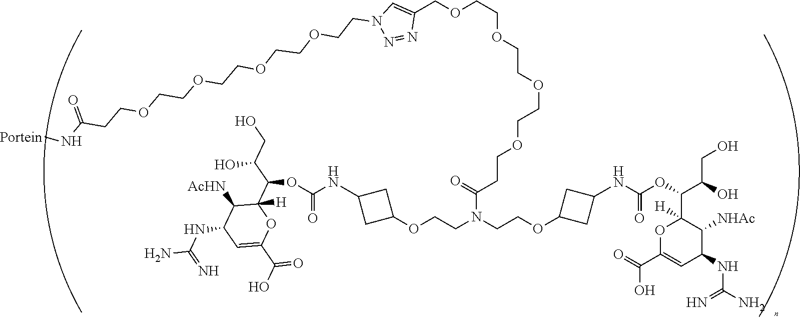 |
| C-20 | 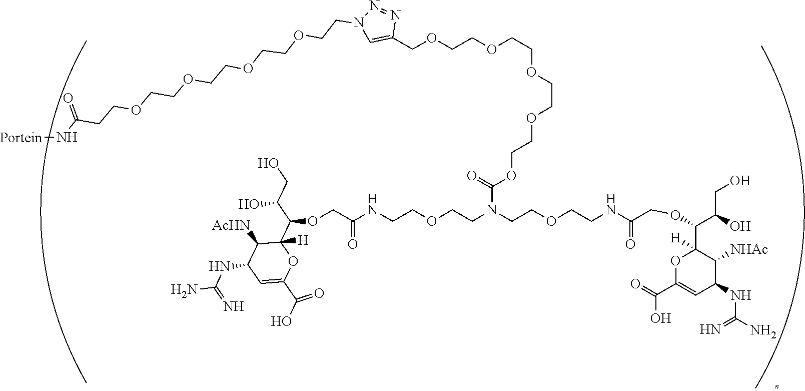 |
| C-21 | 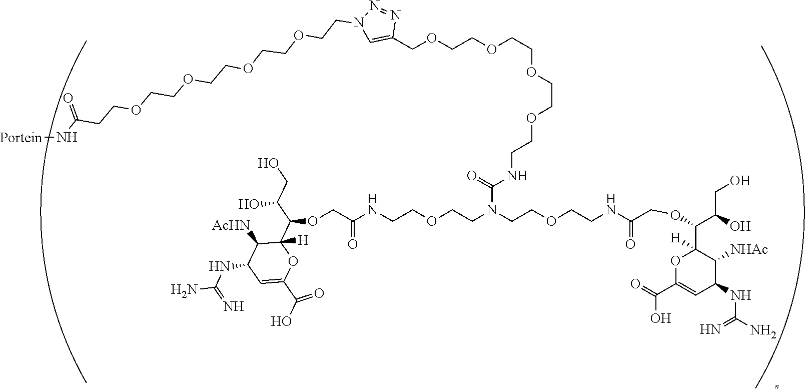 |
| C-22 | 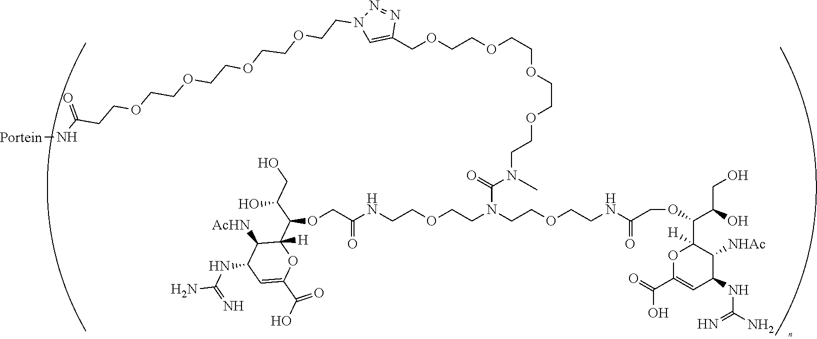 |
| C-23 | 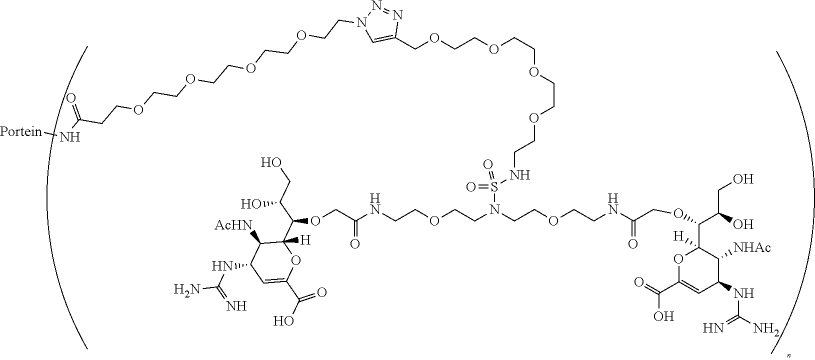 |
| C-24 | 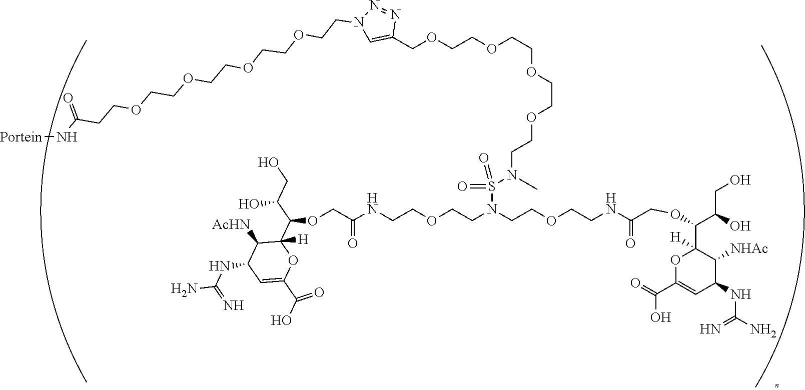 |
| C-25 | 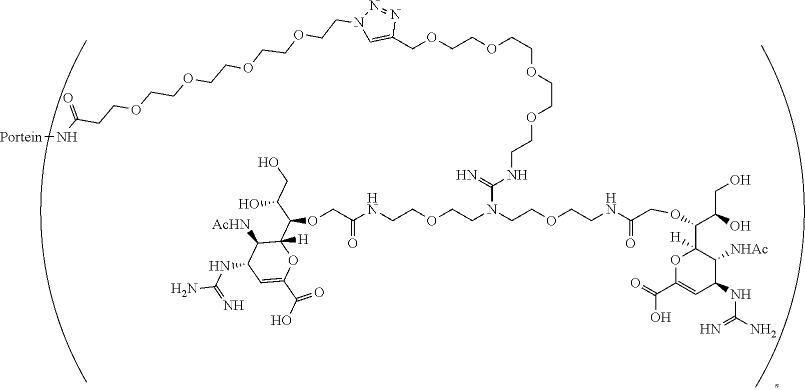 |
| C-26 | 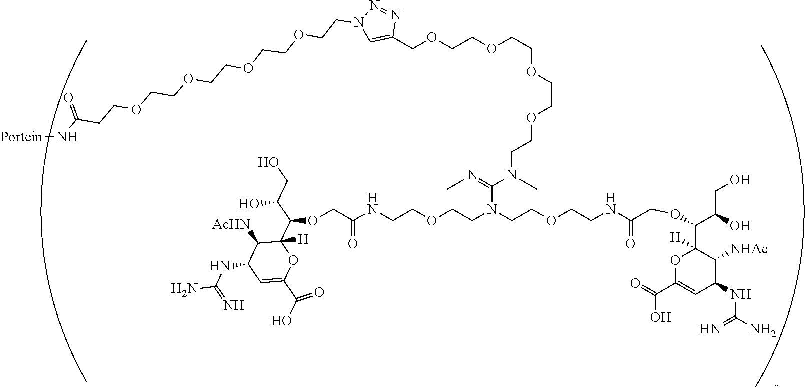 |
| C-27 | 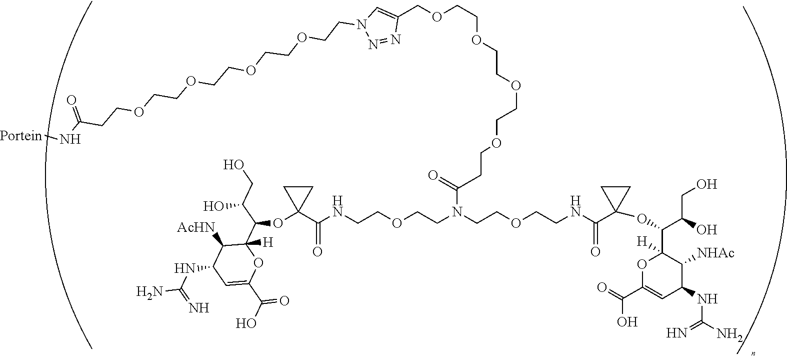 |
| C-28 | 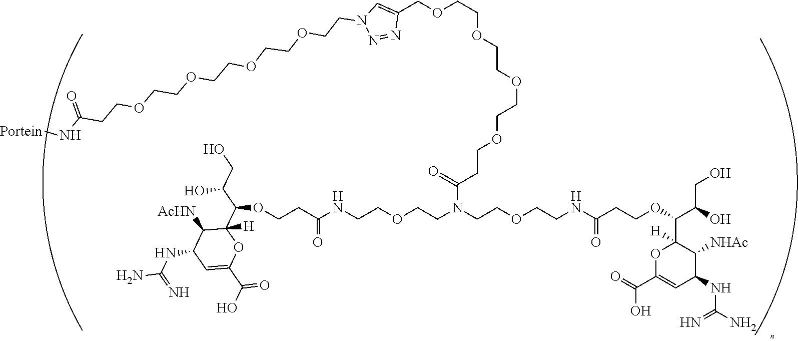 |
| C-29 | 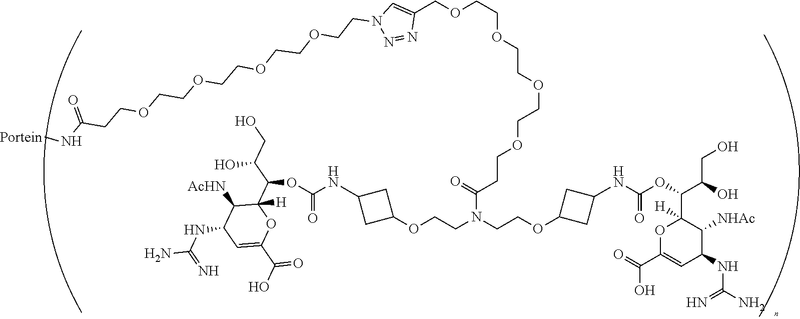 |
| C-30 | 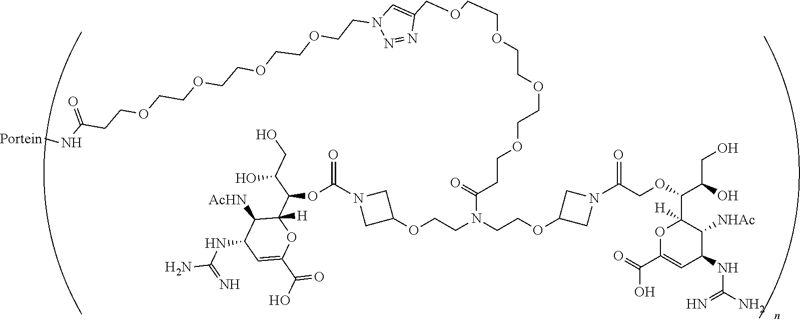 |
| C-31 | 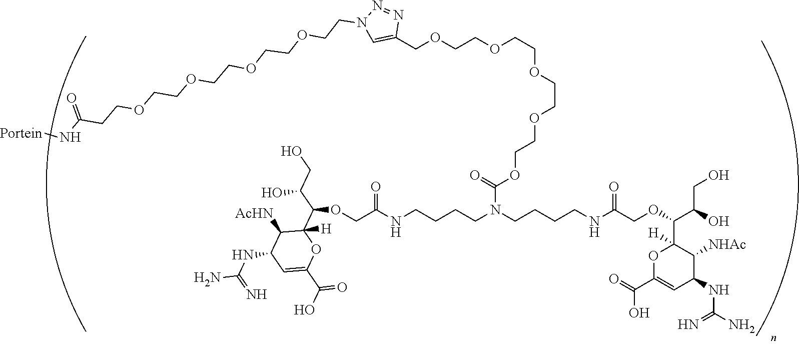 |
| C-32 | 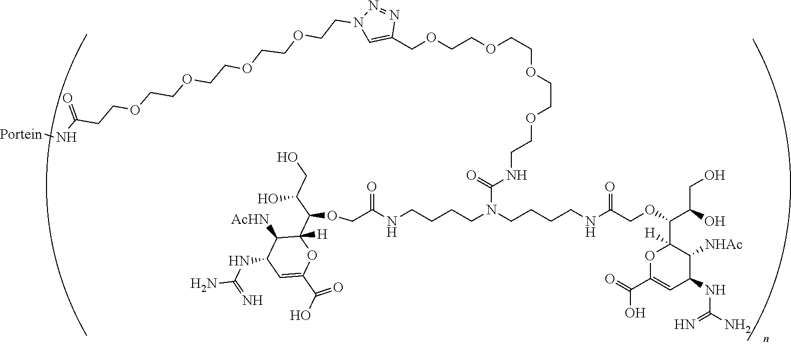 |
| C-33 | 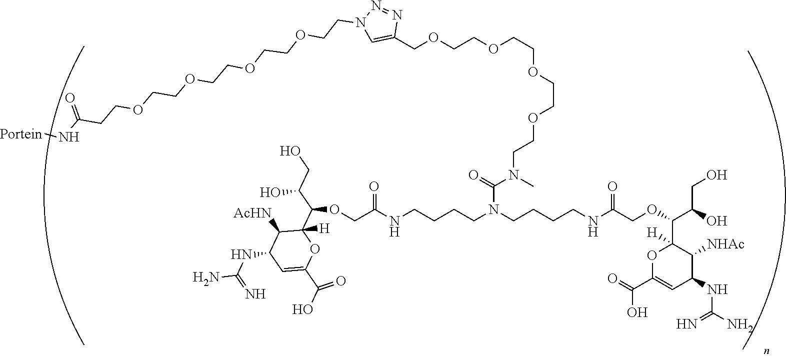 |
| C-34 | 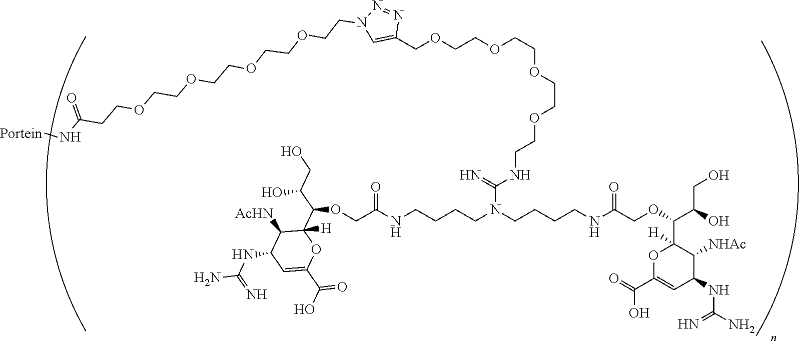 |
| C-35 | 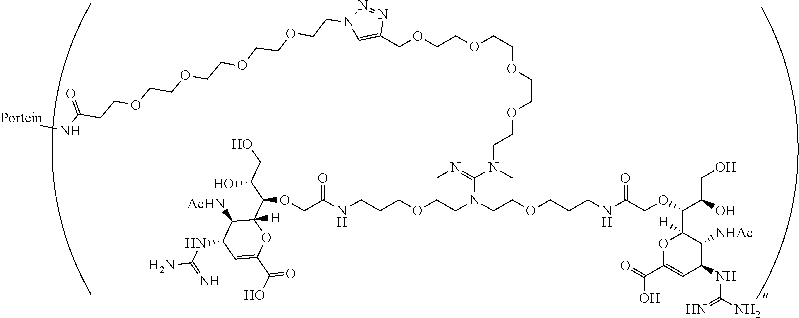 |
| C-36 | 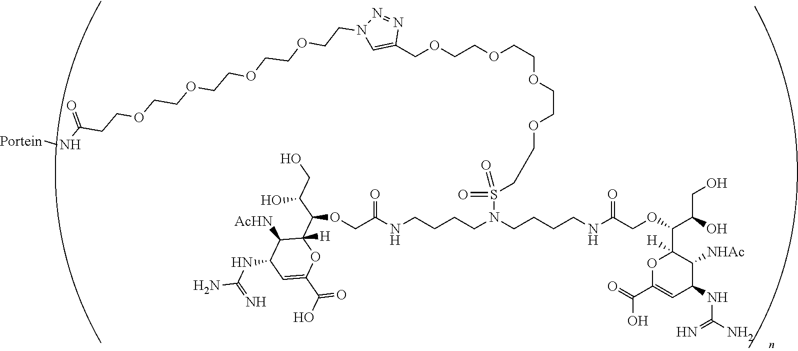 |
| C-37 | 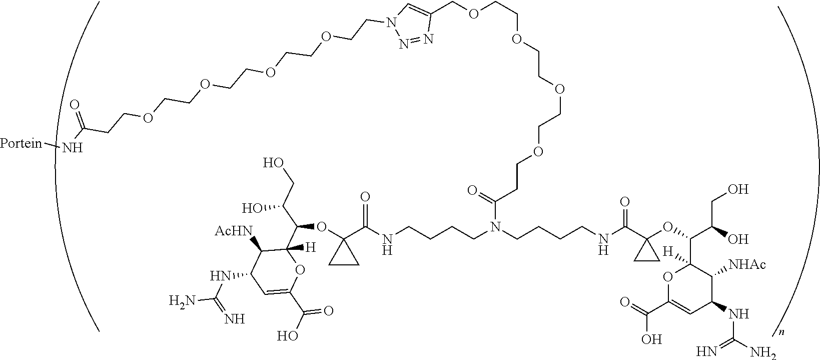 |
| C-38 | 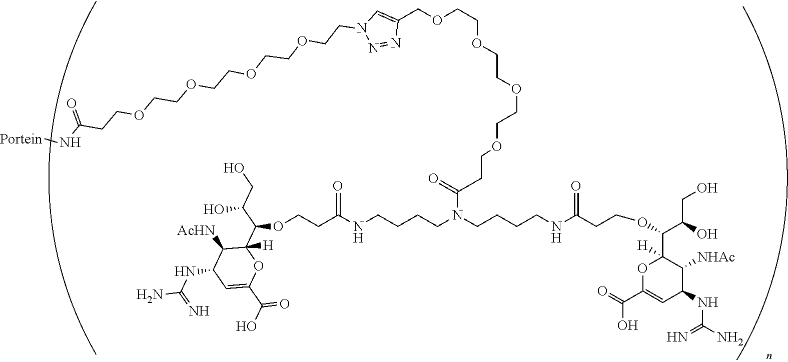 |
| C-39 | 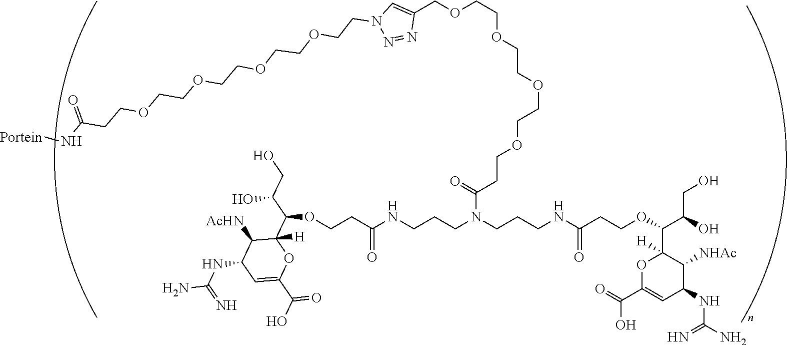 |
| C-40 | 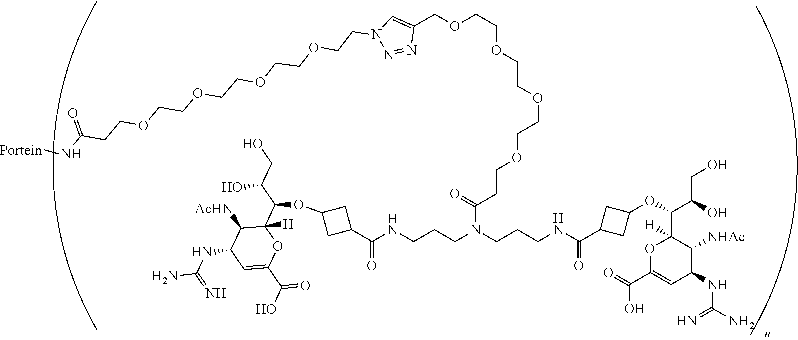 |
| C-41 | 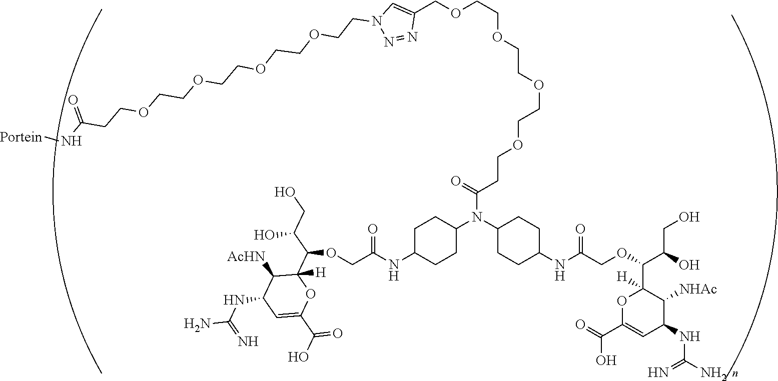 |
| C-42 | 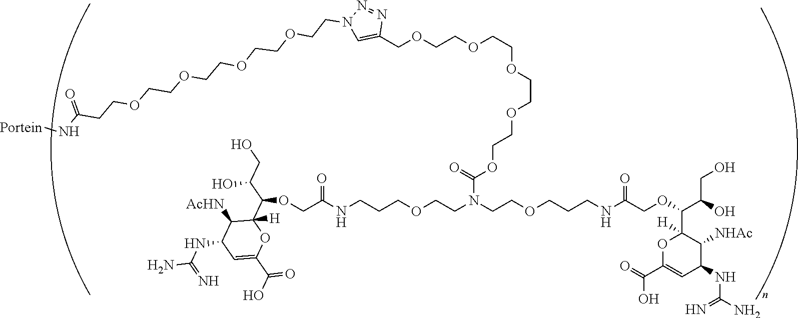 |
| C-43 | 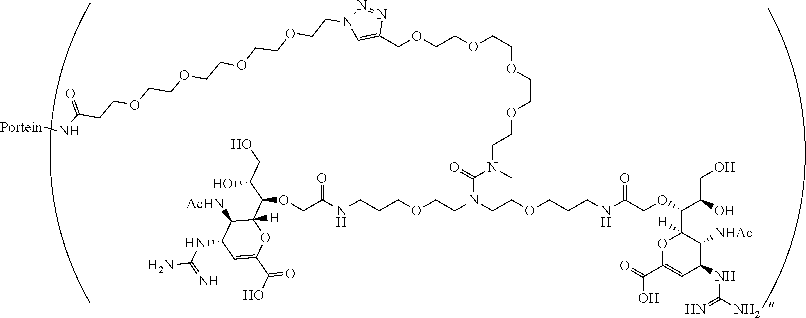 |
| C-44 | 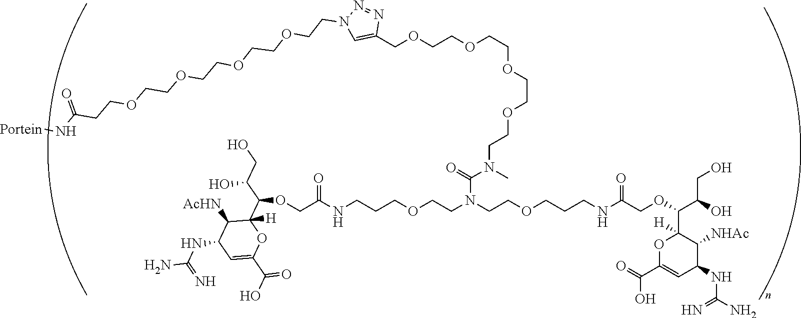 |
| C-45 | 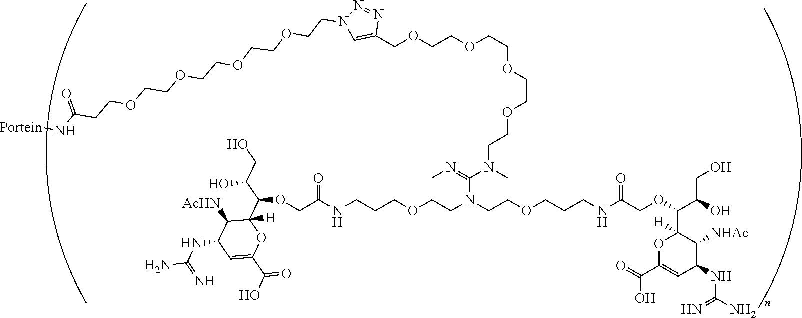 |
| C-46 | 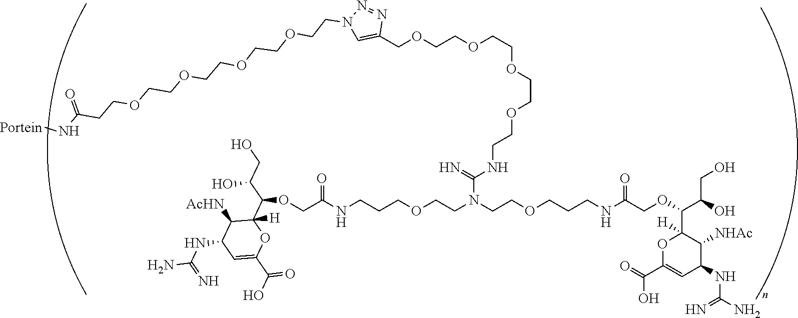 |
| C-47 | 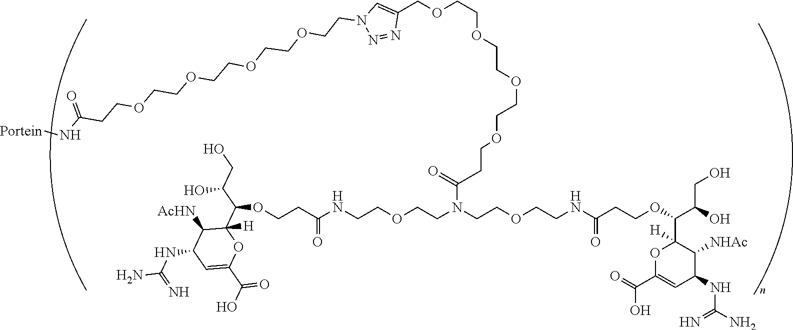 |
| C-48 | 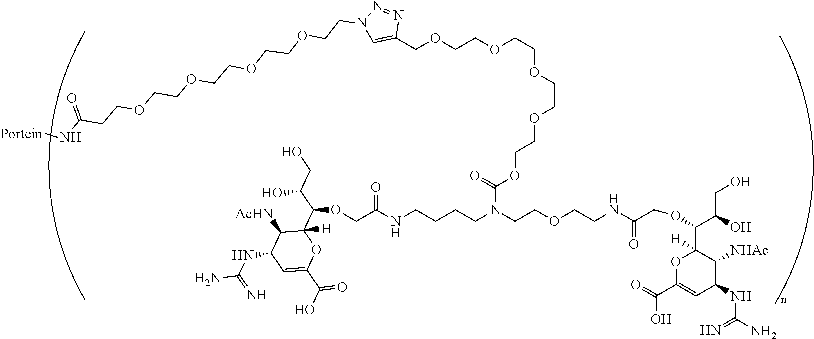 |
| C-49 | 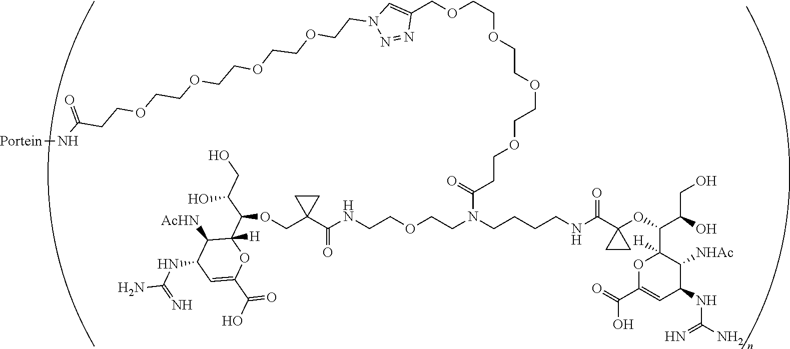 |
| C-50 | 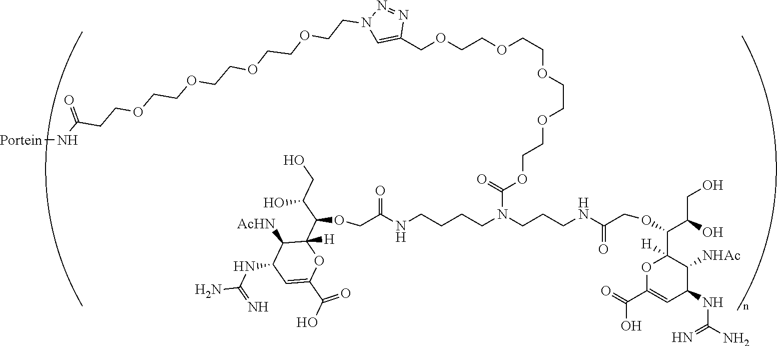 |
| C-51 | 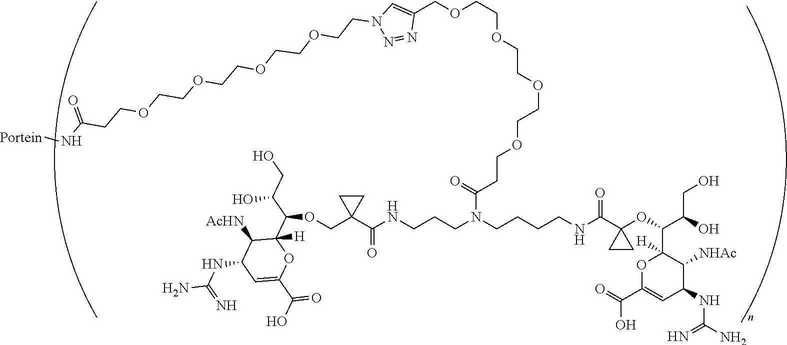 |
| C-52 | 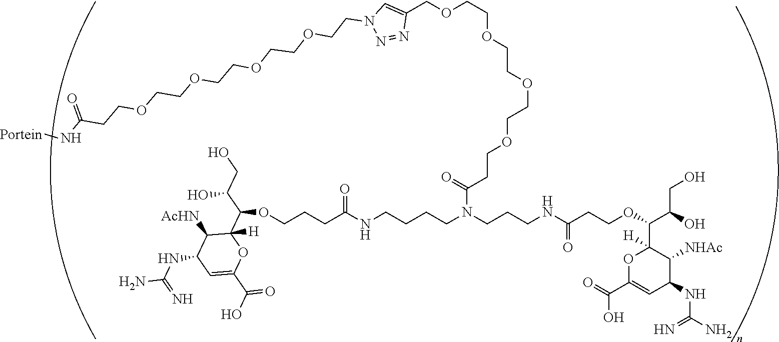 |
| C-53 | 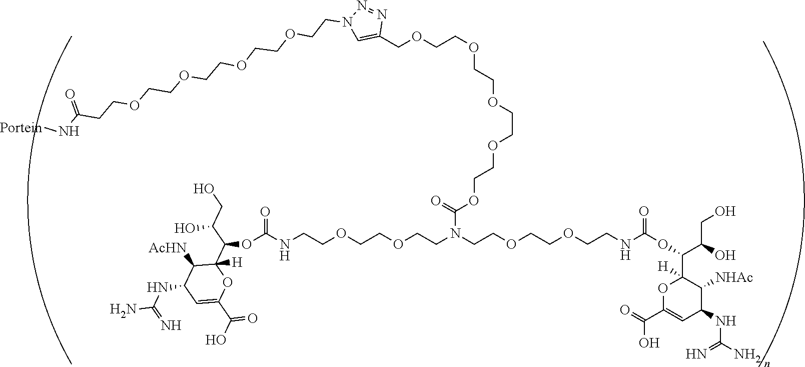 |
| C-54 | 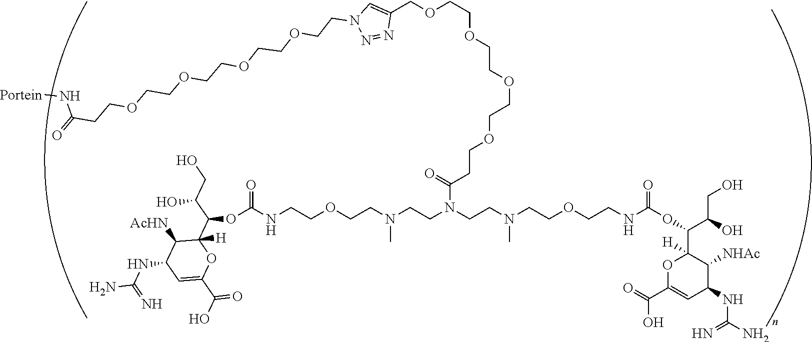 |
| C-55 | 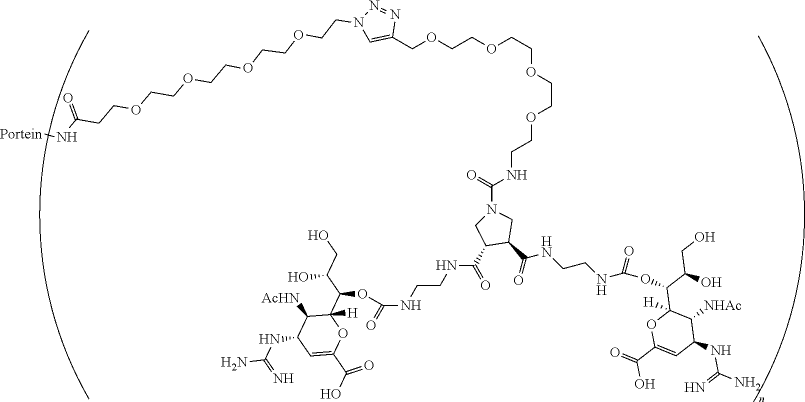 |
| C-56 | 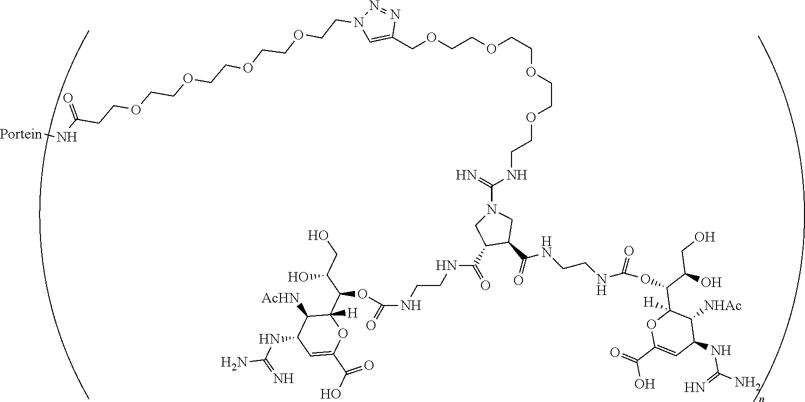 |
| C-57 | 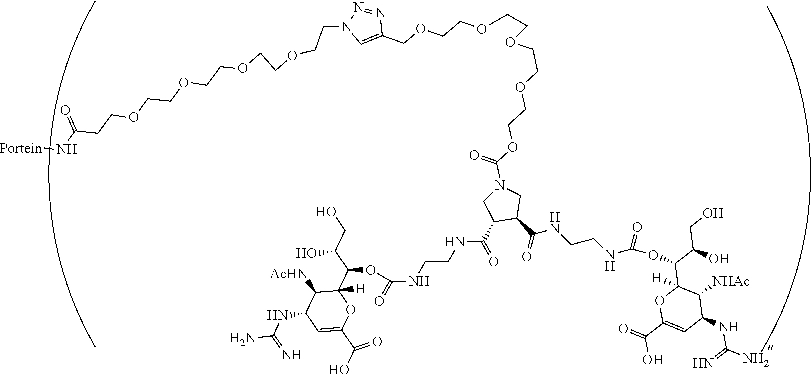 |
| C-58 | 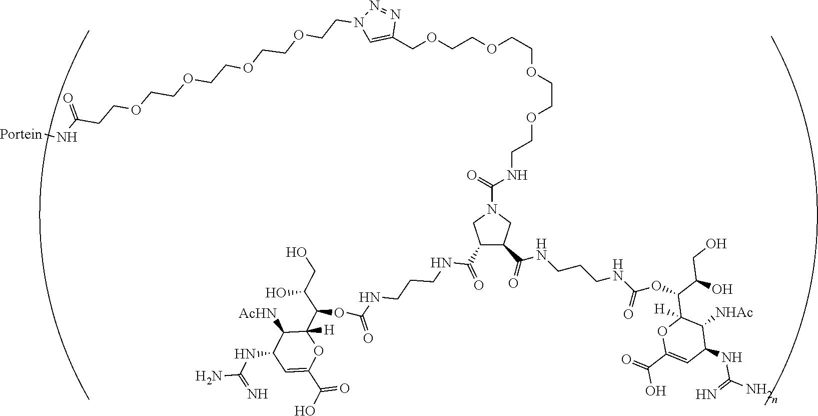 |
| C-59 | 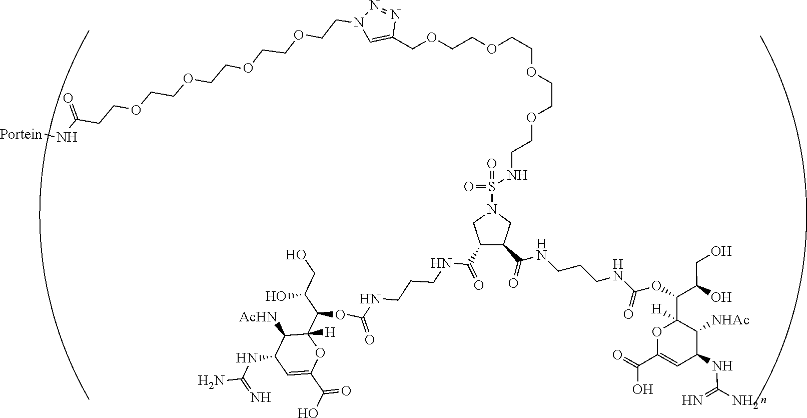 |
| C-60 | 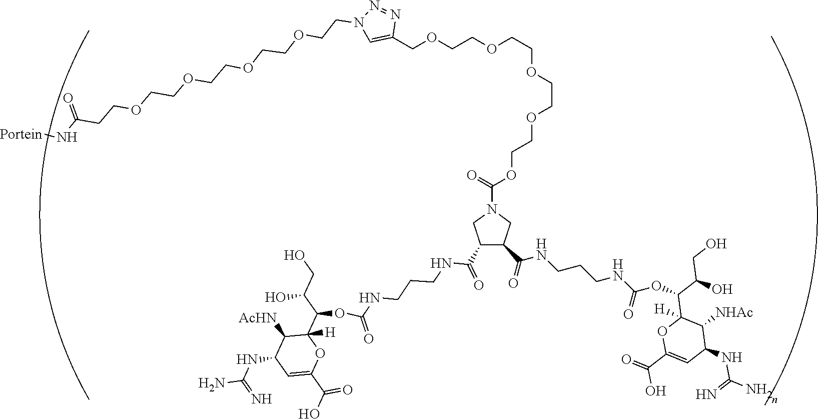 |
| C-61 | 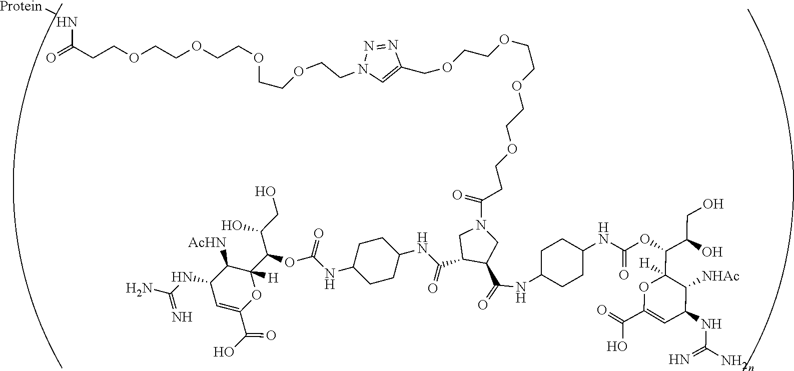 |
| C-62 | 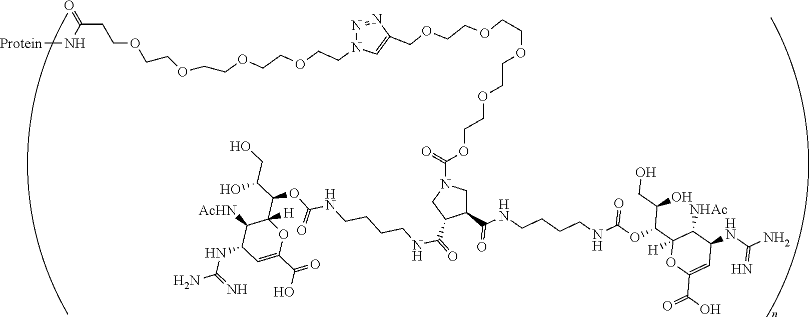 |
| C-63 | 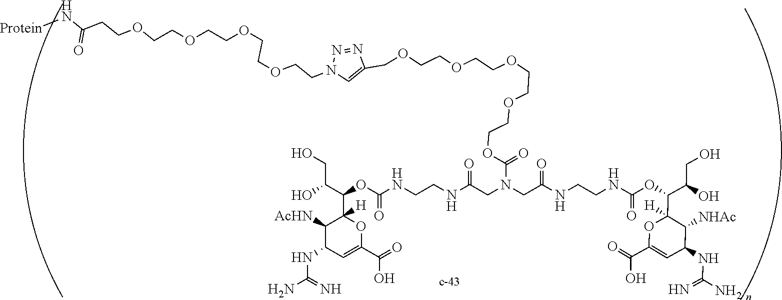 |
| C-64 | 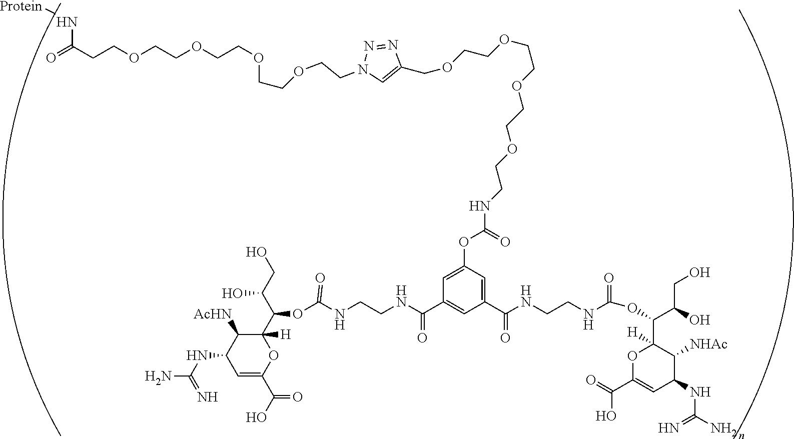 |
| C-65 | 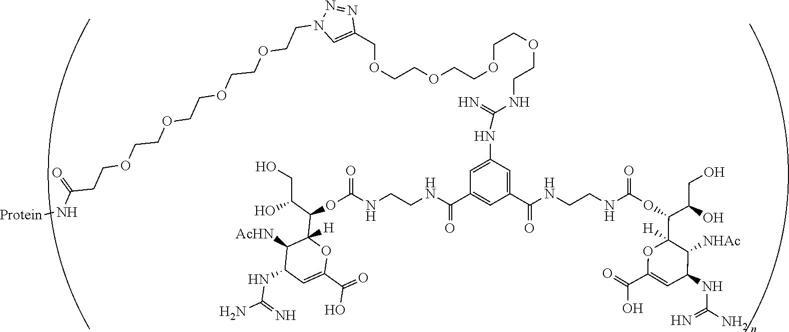 |
| C-66 | 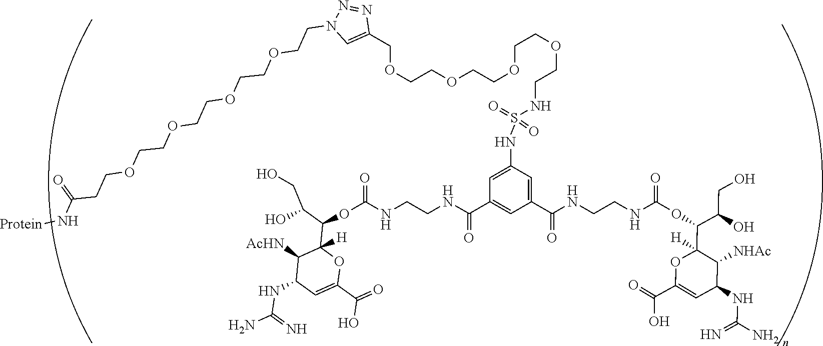 |
| C-67 | 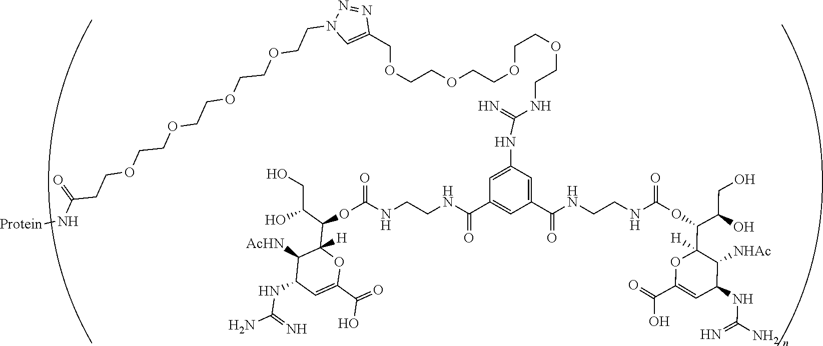 |
| C-68 | 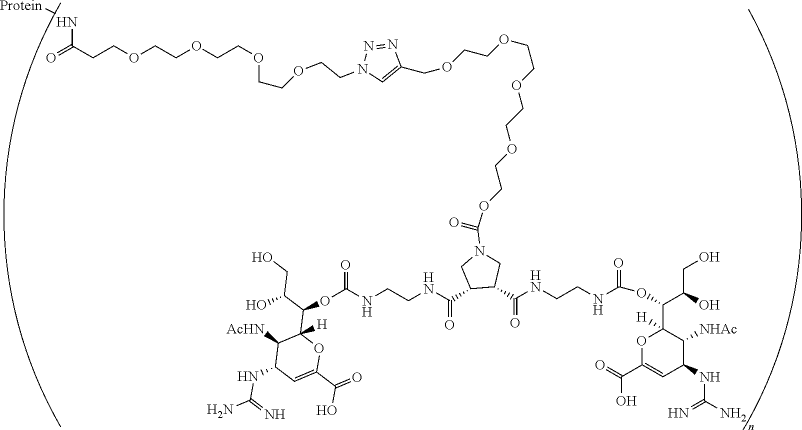 |
| C-69 | 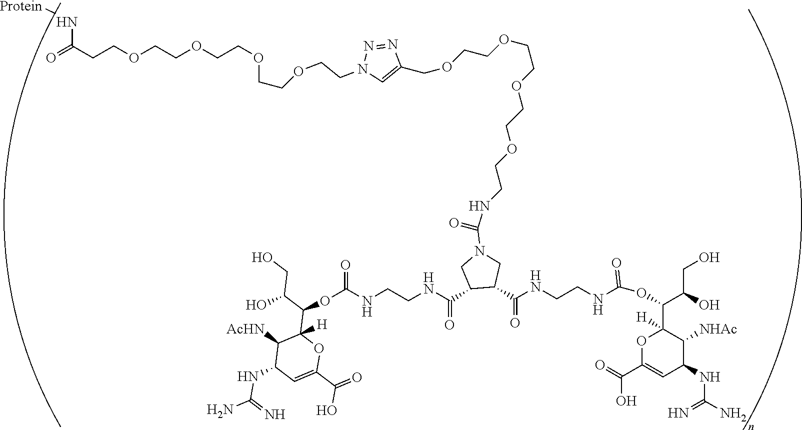 |
| C-70 | 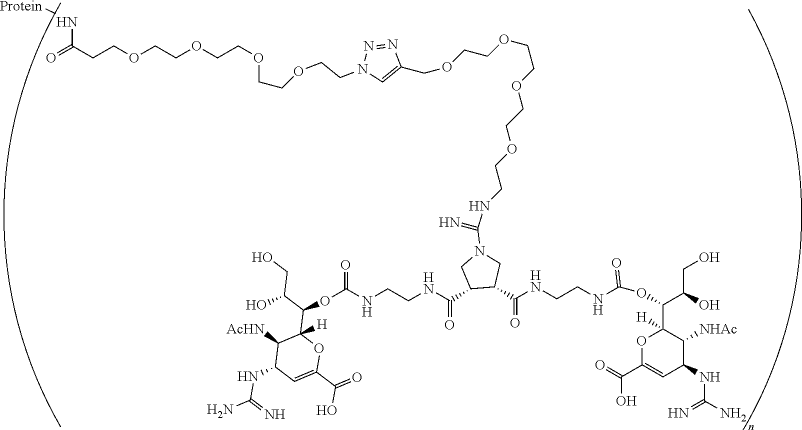 |
| C-71 | 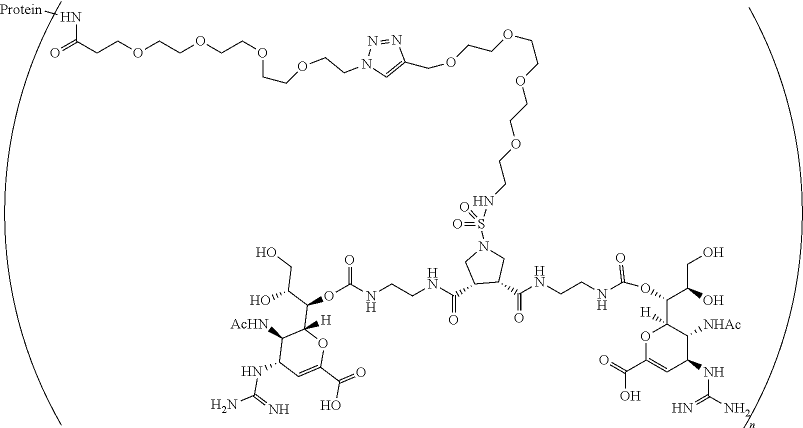 |
| C-72 | 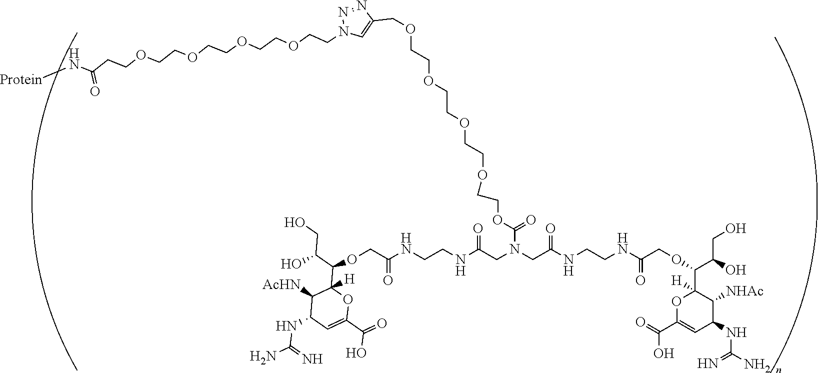 |
| C-73 | 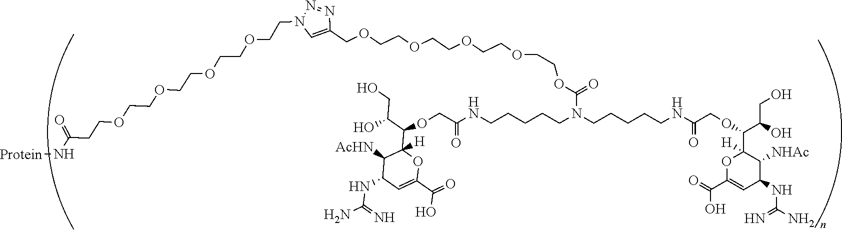 |
| C-74 | 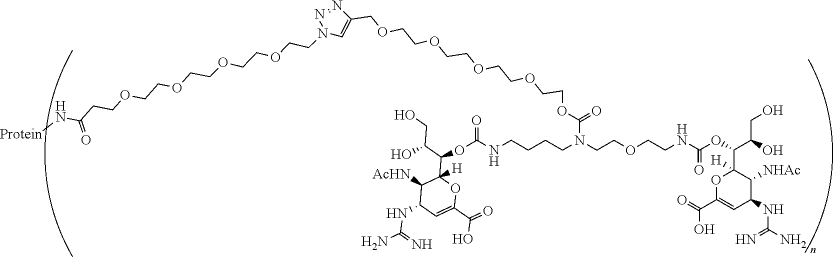 |
| C-75 | 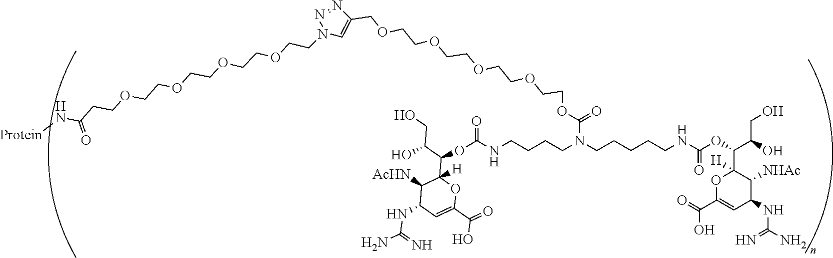 |
| C-76 | 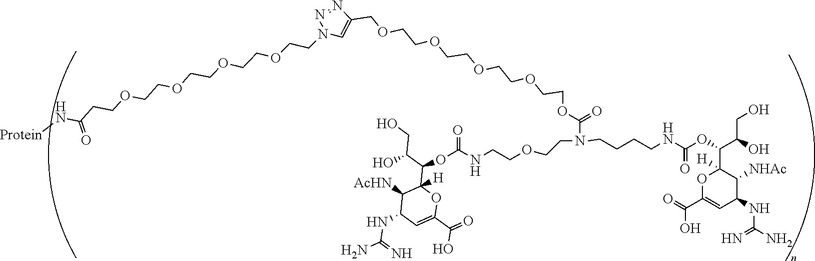 |
| C-77 | 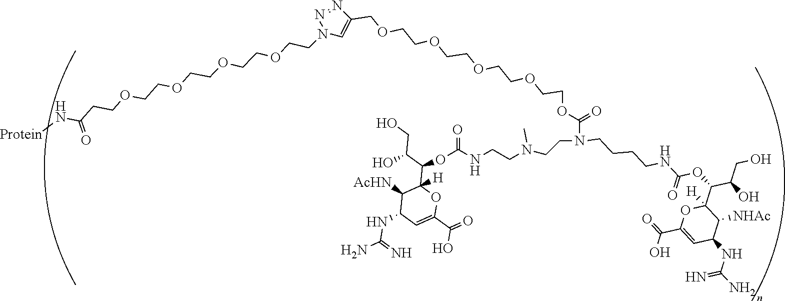 |
| C-78 | 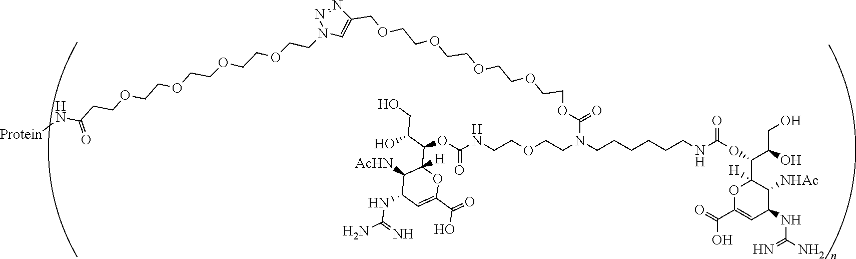 |
| C-79 | 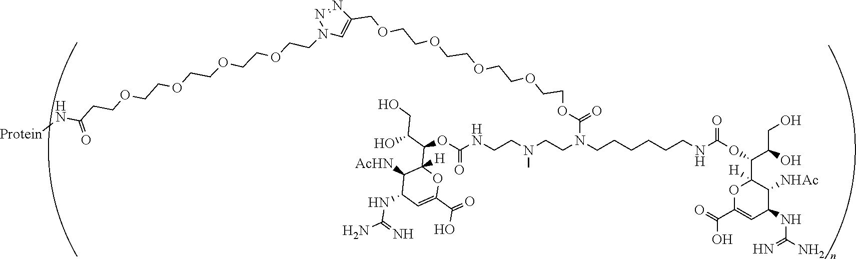 |
| C-80 | 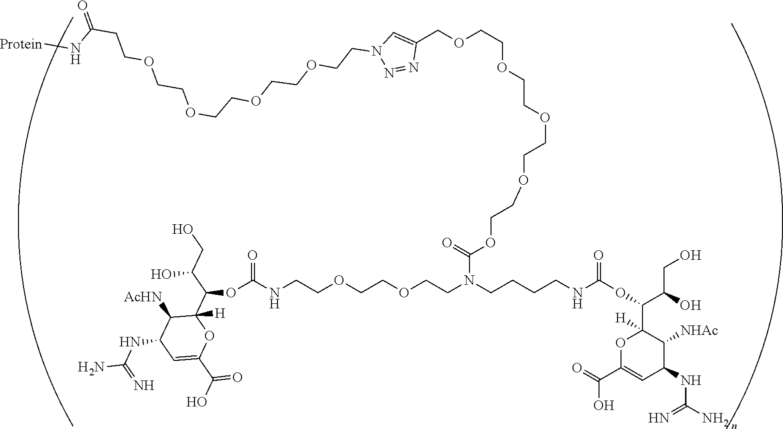 |
| C-81 | 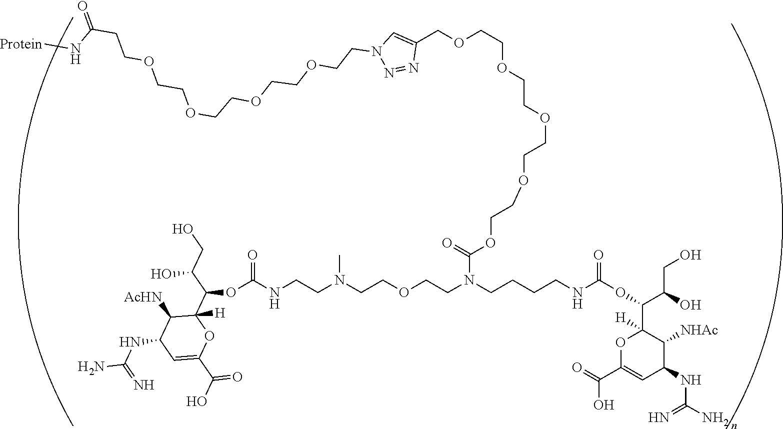 |
| C-82 | 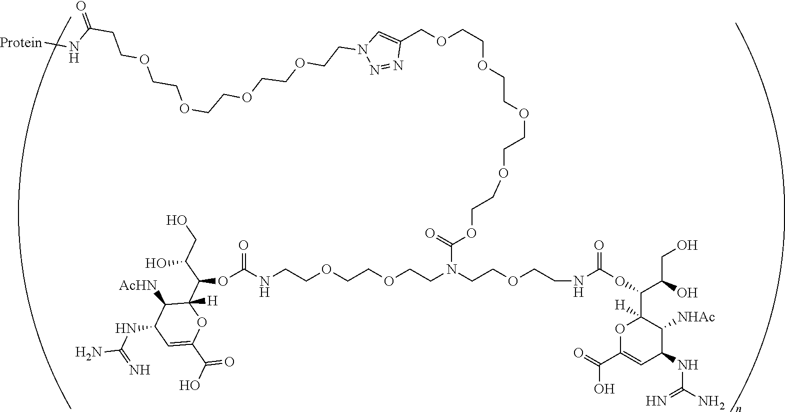 |
| C-83 | 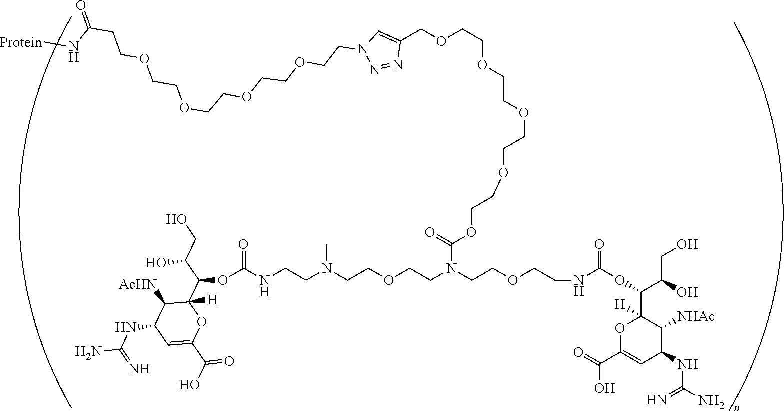 |
| C-84 | 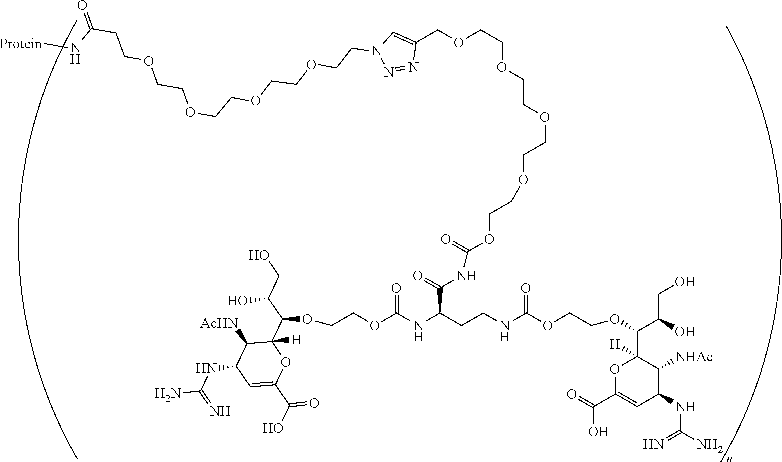 |
| C-85 | 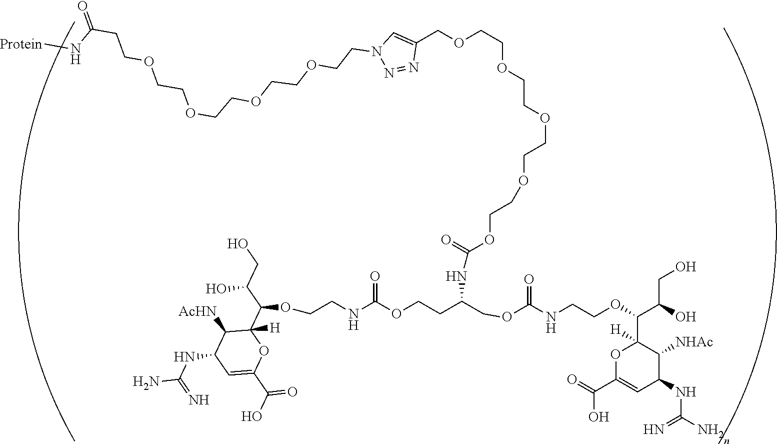 |
| C-86 | 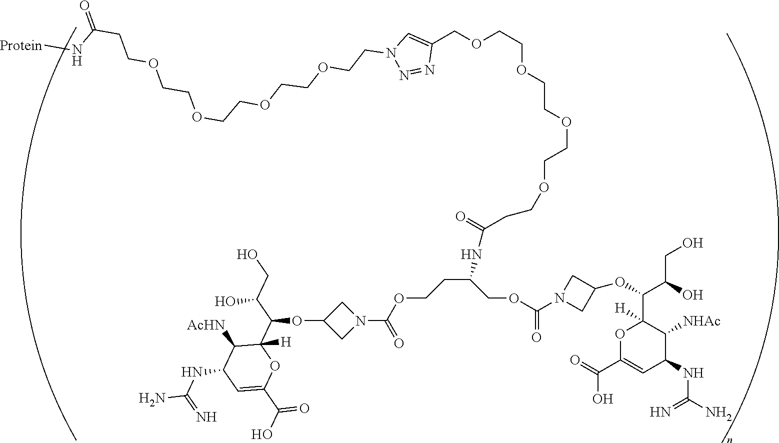 |
| C-87 | 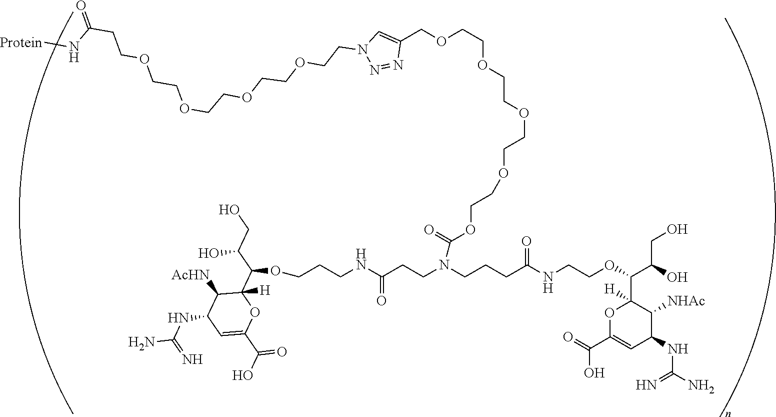 |
| C-88 | 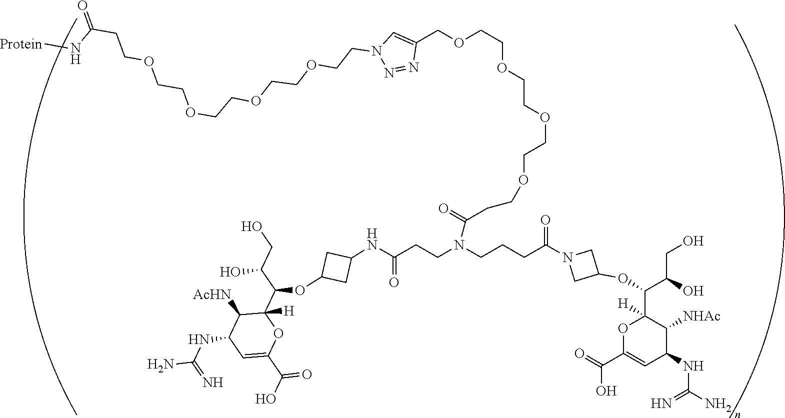 |
| C-89 | 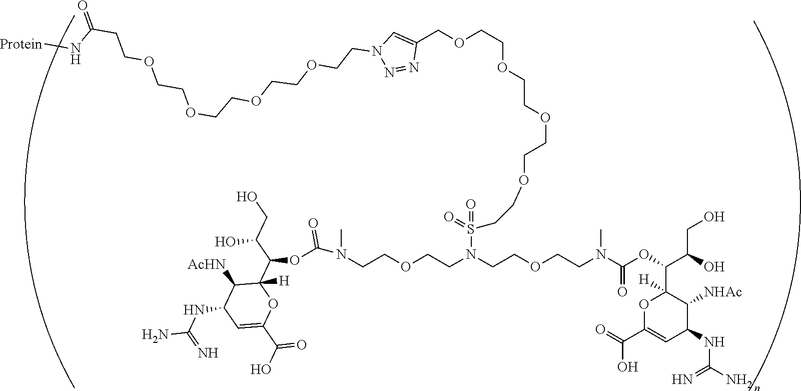 |
| C-90 | 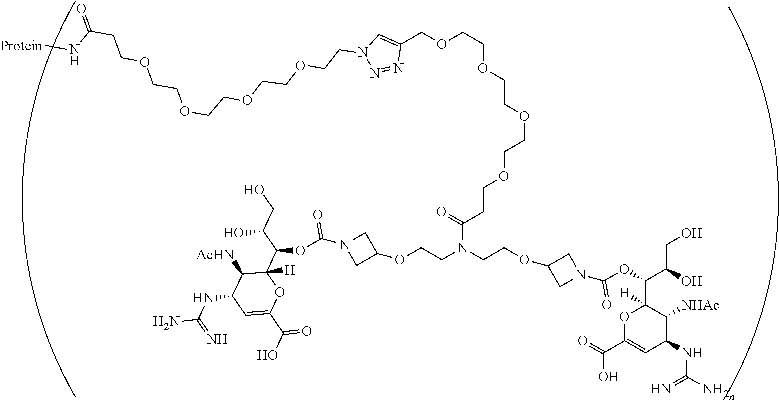 |
| C-91 | 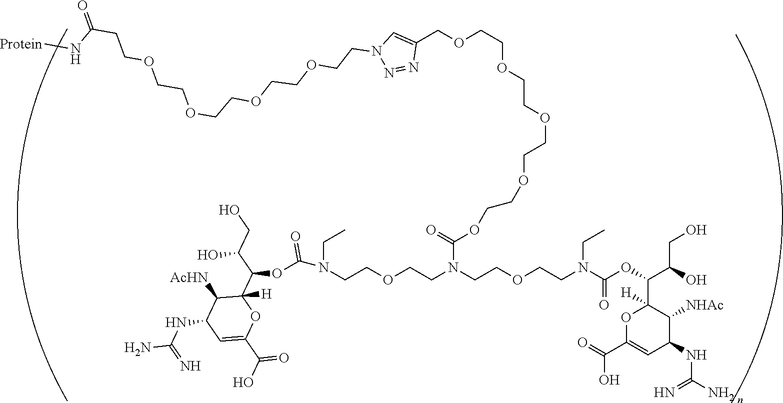 |
| C-92 | 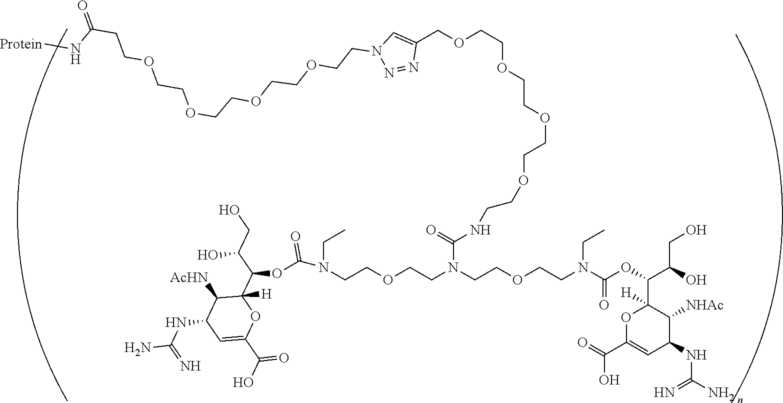 |
| C-93 | 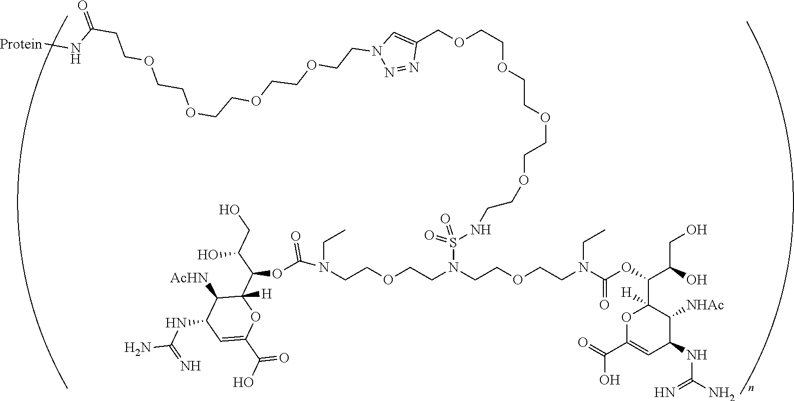 |
| C-94 | 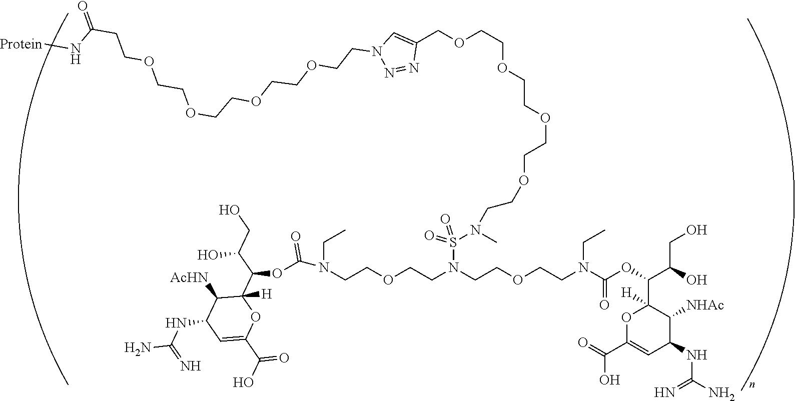 |
| C-95 | 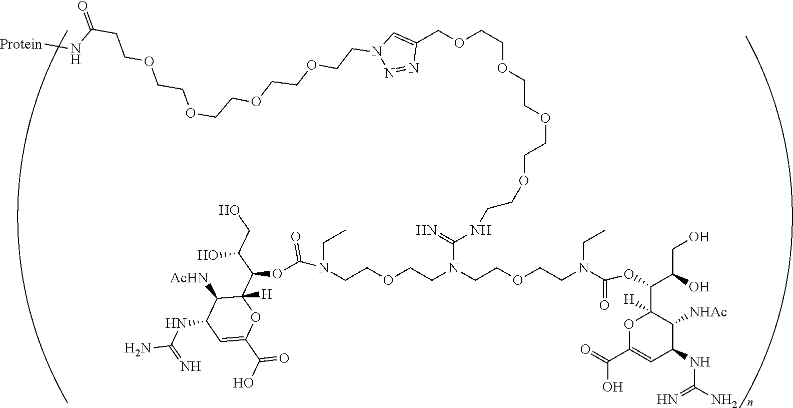 |
| C-96 | 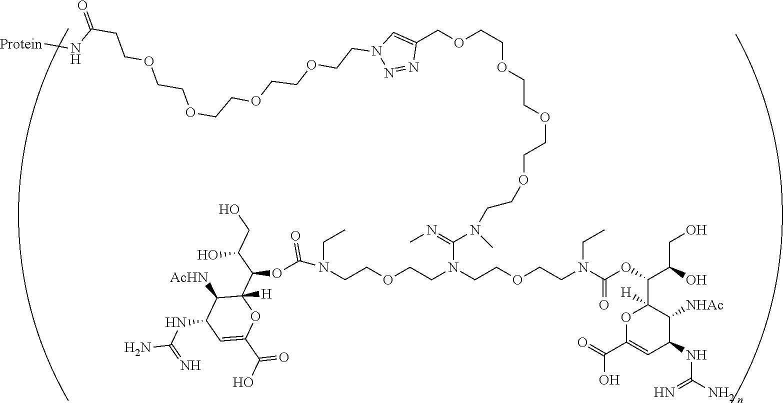 |
| C-97 | 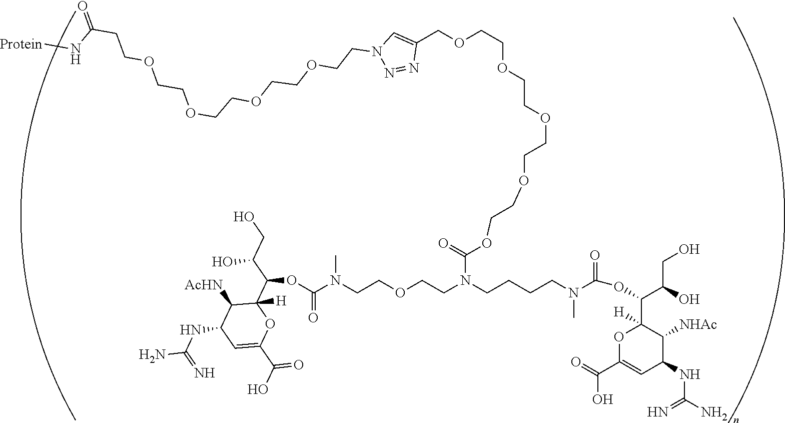 |
| C-98 | 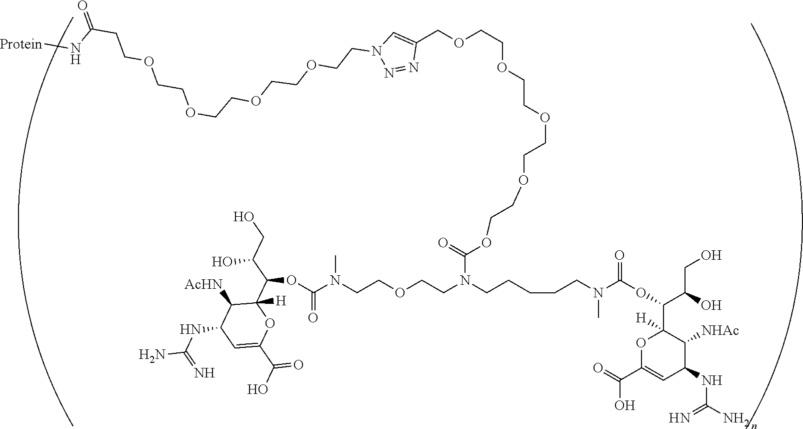 |
| C-99 | 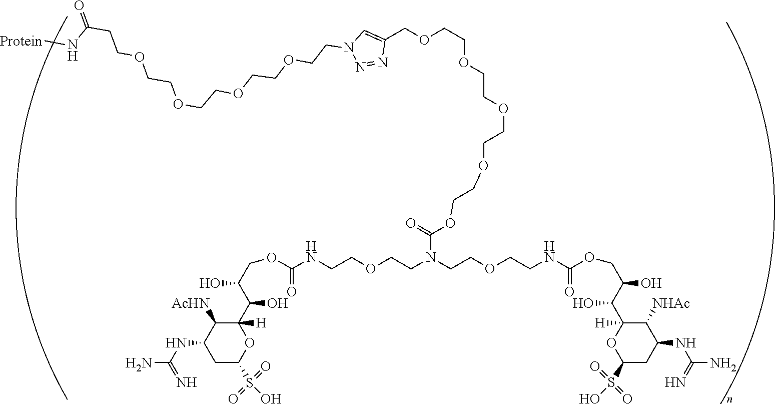 |
| C-100 | 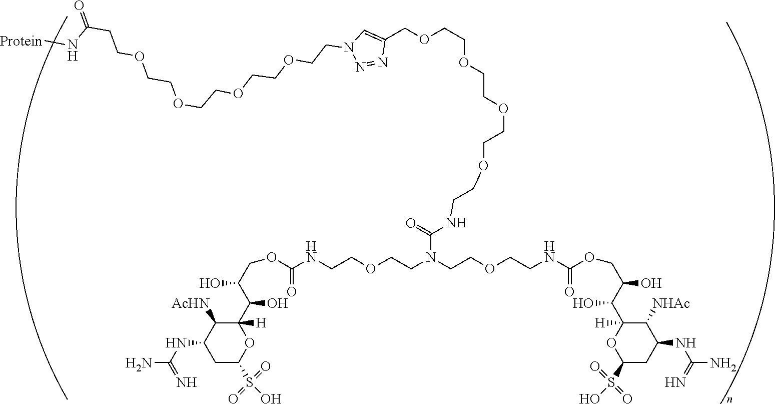 |
| C-101 | 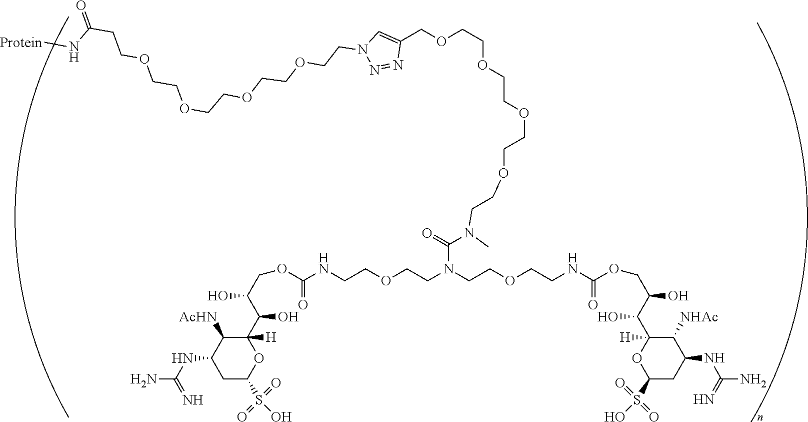 |
| C-102 | 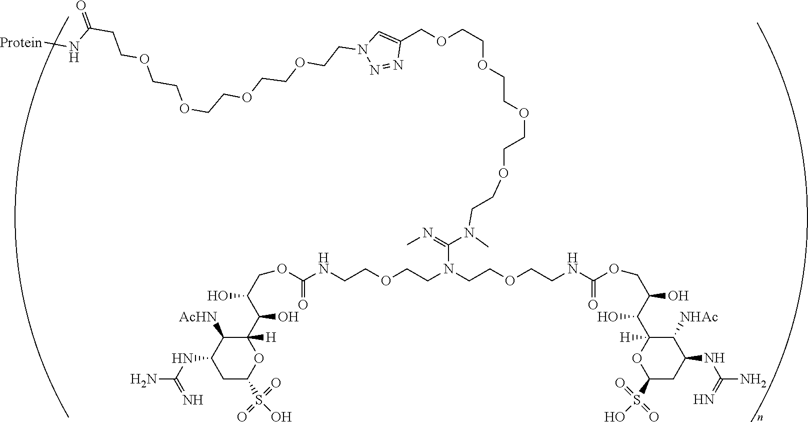 |
| C-103 | 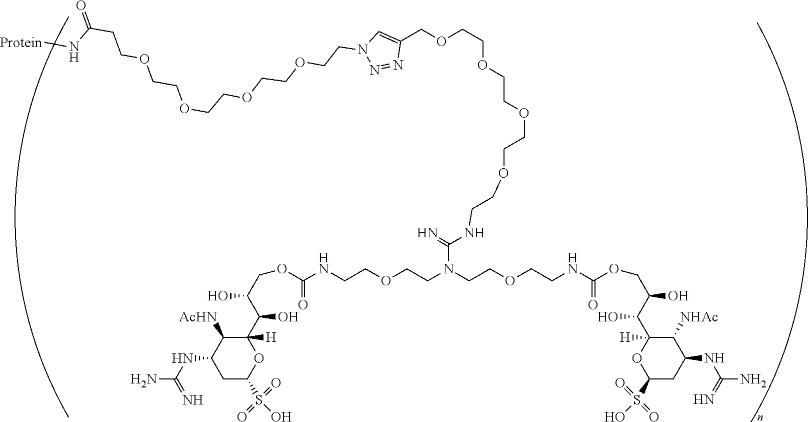 |
| C-104 | 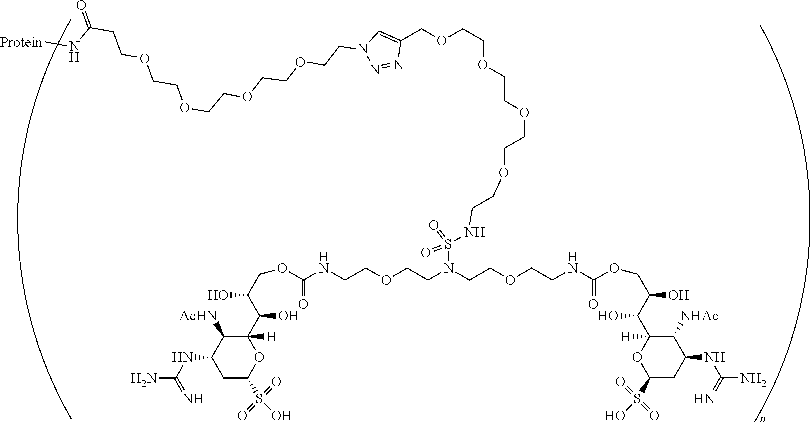 |
| C-105 | 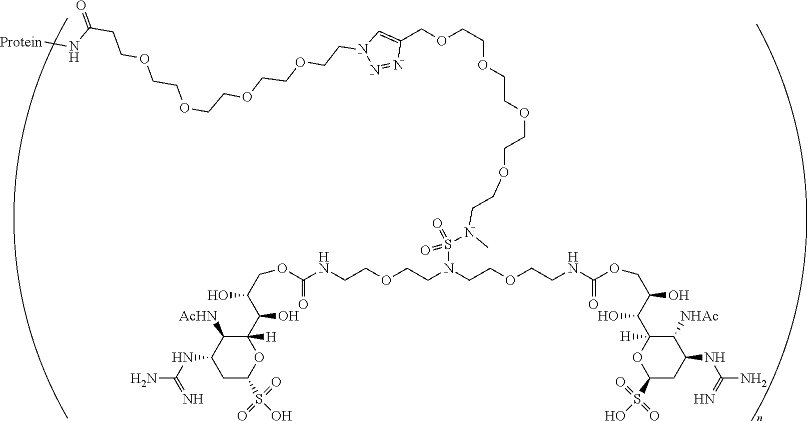 |
| C-106 | 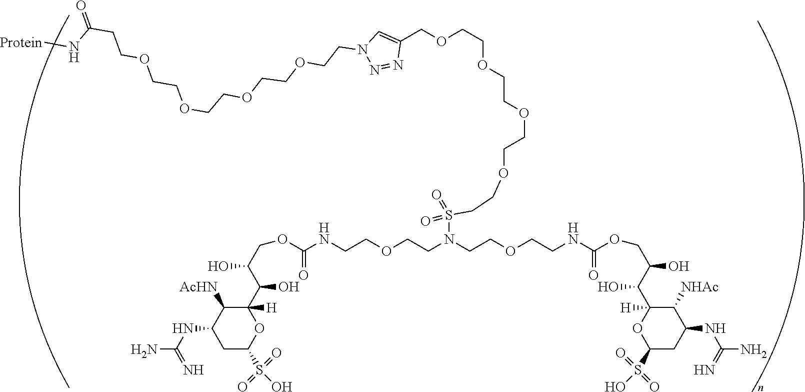 |
| C-107 | 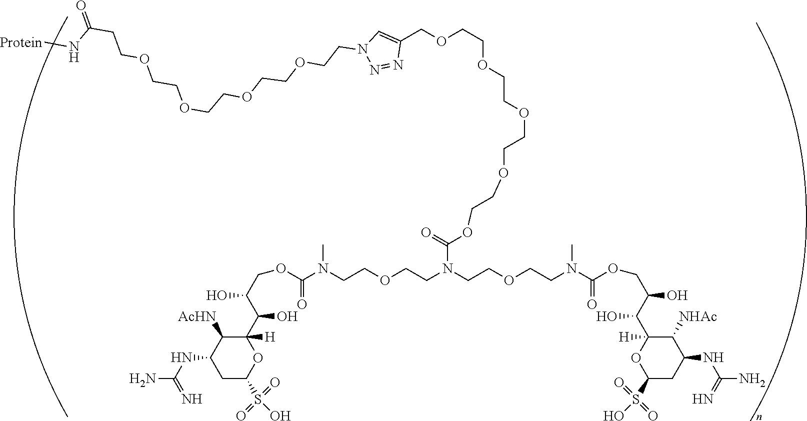 |
| C-108 | 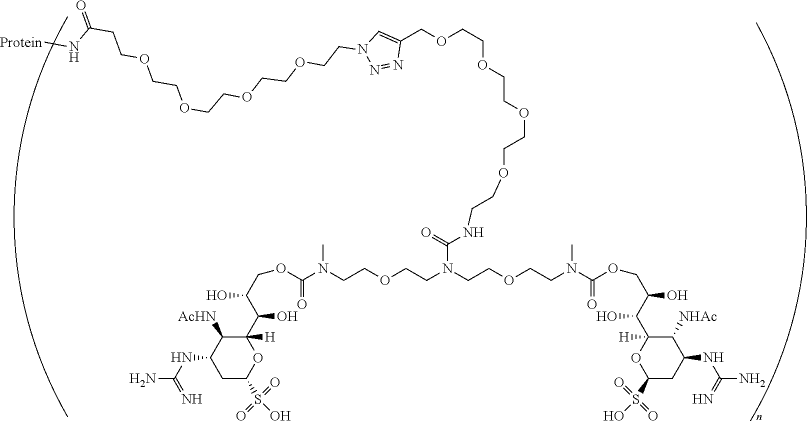 |
| C-109 | 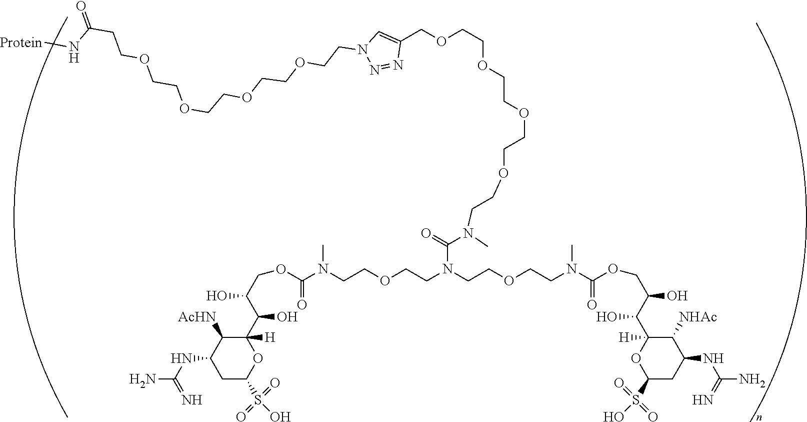 |
| C-110 | 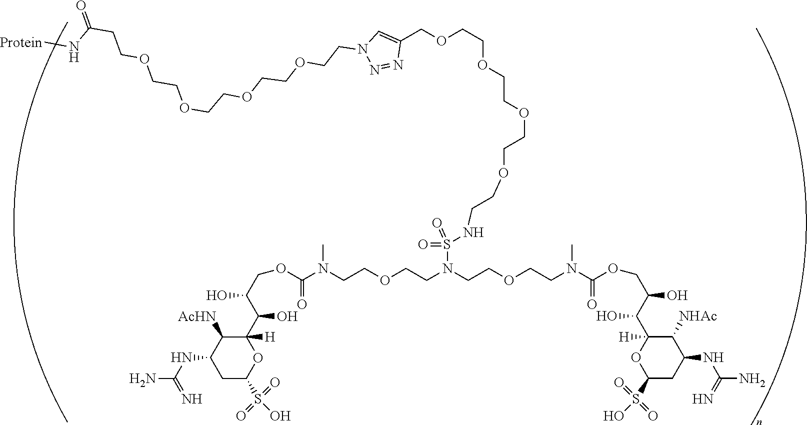 |
| C-111 | 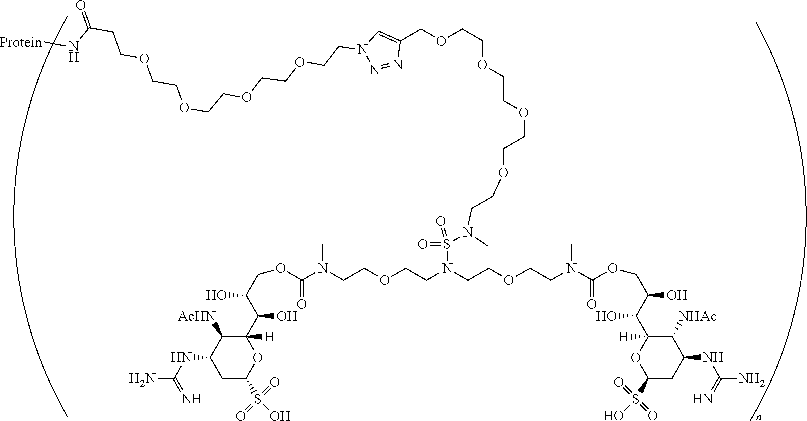 |
| C-112 | 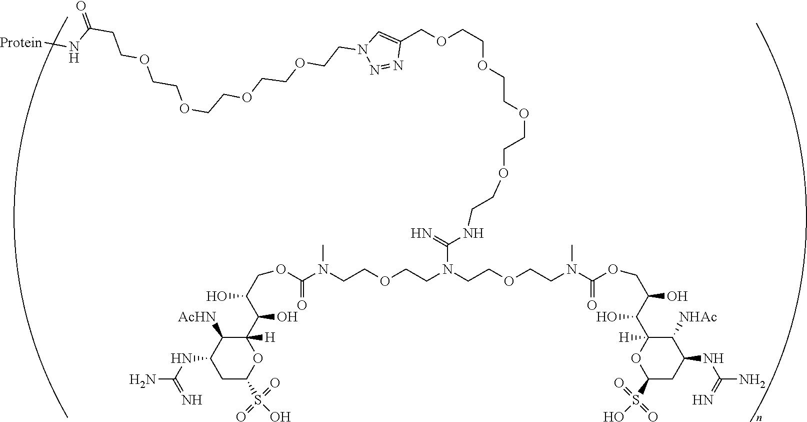 |
| C-113 | 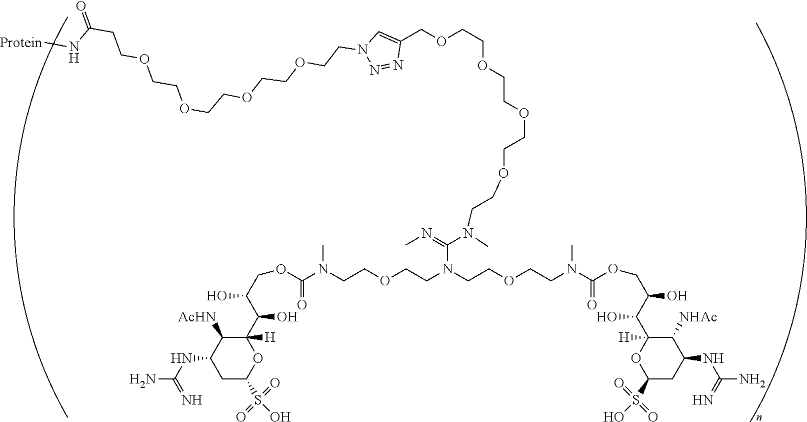 |
| C-114 | 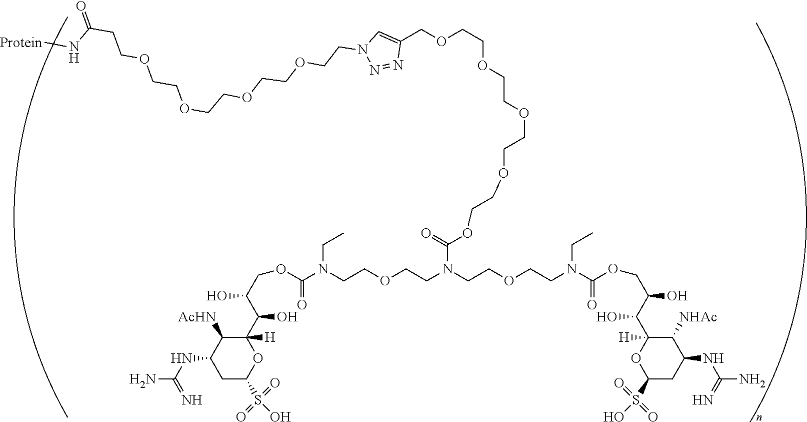 |
| C-115 | 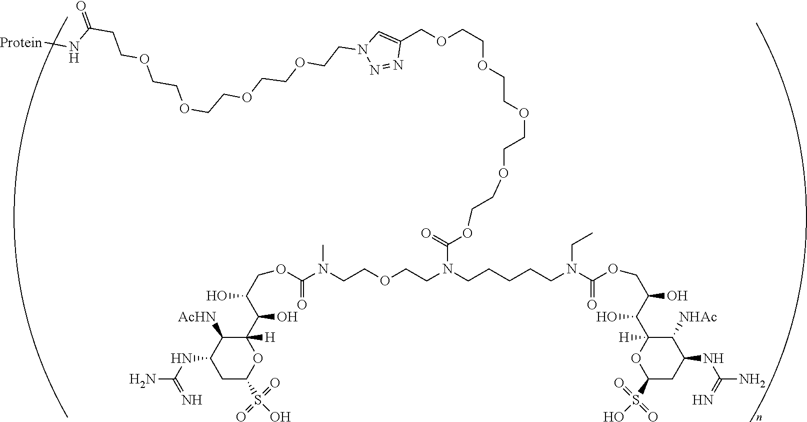 |

where n is as defined above, and protein is E as defined above.

In some embodiments, for the structure of formula I, the ratio of n to m is from 1 to 20, preferably from 2 to 10, such as 2, 3, 4, 5, 6, 7, 8, 9 or 10.

In some embodiments, the conjugate or a pharmaceutically acceptable salt, ester, isomer, solvate, prodrug or isotopically labeled derivative thereof, has an average DAR value between 0.5 and 10.0.

In some embodiments, E is an antibody or antibody fragment, Fc structural domain monomer, Fc structural domain, and/or Fc binding peptide. In some embodiments, E comprises or consists of an amino acid sequence of any one of SEQ ID Nos. 1-68 or an amino acid sequence that is at least 9500 identical to any one of SEQ TD Nos. 1-68. In some embodiments, antibody fragments are antigen-binding fragments, such as

In some embodiments, the antibody or antibody fragment is a human, mouse, camelid, goat, sheep, rabbit, chicken, guinea pig, hamster, horse or rat antibody or antibody fragment.

In some embodiments, the antibody or antibody fragment is of the IgG, IgA, IgD, IgE, or IgM type.

In some embodiments, the antibody fragment includes scFv, sdAb, Fab, Fab′, Fab′2, F(ab′)2, Fd, Fv, Feb or SMIP.

In some embodiments, the antibody or antibody fragment is able to recognize surface antigens of viruses, such as CR6261, CR8020, MEDI8897, Palivizumab, SD38, and so on.

In some embodiments, the Fc structural domain monomer may be an Fc structural domain monomer of an antibody subtype (such as, IGHG1*01 (such as G1m(za)), IGHG1*07 (such as G1m(zax)), IGHG1*04 (such as G1m(zav)), IGHG1*03 (G1m(f)), IGHG1*08 (such as G1m(fa)), IGHG2*01, IGHG2*02, IGHG2*06, IGHG3*01, IGHG3*04, IGHG3*05, IGHG3*09, IGHG3*10, IGHG3*11, IGHG3*12, IGHG3*06, IGHG3*07, IGHG3*08, IGHG3*13, IGHG3*03, IGHG3*14, IGHG3*15, IGHG3*16, IGHG3*17, IGHG3*18, IGHG3*19, IGHG2*04, IGHG4*01, IGHG4*02, IGHG4*03)(Vidarsson et al, IgG subclasses and allotypes: from structure to effector function. Frontiers in Immunology. 5(520):1-17(2014)) of any kind of immunoglobulins. Fc structural domain monomers may contain one or more unnatural amino acid sequences that serve as targeted coupling sites for small molecule drugs. Fc structural domain monomers may contain one or more solvent-exposed cysteine or lysine residues that have been modified in a targeted position to provide additional coupling sites for small molecule drugs.

In some embodiments, the Asn amino acid residue in E is replaced with an Ala amino acid residue to avoid coupling of the site.

In some embodiments, E includes additional Cys amino acid residues to increase the coupling site without affecting the spatial three-dimensional structure of the antibody protein.

In some embodiments, the end of E contains additional amino acid sequences, such as a protein purification tag (e.g., six histidine tags), or a signal peptide sequence (e.g., a signal peptide sequence of human interleukin 2) or an MVRS amino acid sequence or an ISAMVRS amino acid sequence.

In some embodiments, the protein purification tag is located at the C-terminus of E. In some embodiments, the signal peptide sequence is located at the N-terminus of E.

In some embodiments, the protein purification tag is selected from a six of histidine tag or a c-Myc tag. In some embodiments, the signal peptide is selected from a human IL-2 signal peptide sequence (e.g., MYRMQLLSCIALSLALVTNS (SEQ ID NO: 69)), a human serum albumin signal sequence (e.g., MKWVTFISLLFLFSSAYS (SEQ ID NO: 70)), a mouse heavy chain MIgG Vh signal sequence (e.g. MGWSCIILFLVATATGVHS (SEQ ID NO: 71)).

In some embodiments, the N-terminus of E further includes a hinge region or a partial hinge region.

In some embodiments, E includes an amino acid sequence of, or consisting of, any one of SEQ ID NOs:1-68.

In some embodiments, E includes an amino acid sequence having at least 70%, 75%, 80%, 85%, 90%, 95%, 96%, 97%, 98%, 99%, or 100% identity to, or consisting of, any one of SEQ ID NOs:1-68.

In some embodiments, E includes the following amino acid sequences or consists of the following amino acid sequences, said amino acid sequence:
(i) having at least 70%, 75%, 80%, 85%, 90%, 95%, 96%, 97%, 98%, or 99% identity to the amino acid sequence of any one of SEQ ID NOs:1-68; and
(ii) including N-terminal addition sequences, mutations and/or C-terminal addition sequences etc. described for each SEQ ID NO: in the “SEQ ID NOs: 1-68 and descriptions thereof” of present disclosure.

In some embodiments, E further includes a connector. In one embodiment, a connector is a short amino acid sequence consisted of amino acids, such as a glycine (G) and/or a serine (S) and/or a threonine residue (T), alone or in combination. In one embodiment, the connector includes the amino acid sequence (G4S)n, wherein n is an integer equal to or greater than 1, e.g., n is an integer of 1, 2, 3, 4, 5, 6, or 7. In one embodiment, the linker is GGGGS. In one embodiment, the connector includes the amino acid sequence TS(G4S)n, wherein n is an integer equal to or greater than 1, e.g., n is an integer of 2, 3, 4, 5, 6 or 7. In one embodiment, the connector includes the amino acid sequence G(G4S)n, wherein n is an integer equal to or greater than 1, e.g., n is an integer of 2, 3, 4, 5, 6 or 7.

In some embodiments, E includes the amino acid sequence in SEQ ID NO: 1. In some embodiments, E includes the amino acid sequence having at least 70%, 75%, 80%, 85%, 90%, 95%, 96%, 97%, 98%, or 99% identity to the amino acid sequence of SEQ ID NO: 1, and optionally has an IL2 signaling sequence at its N-terminus.

In some embodiments, E includes the amino acid sequence of SEQ ID NO: 2. In some embodiments, E includes the amino acid sequence having at least 70%, 75%, 80%, 85%, 90%, 95%, 96%, 97%, 98%, or 99% identity to the amino acid sequence of SEQ ID NO: 2.

In some embodiments, E includes the amino acid sequence of SEQ ID NO: 3. In some embodiments, E includes the amino acid sequence having at least 70%, 75%, 80%, 85%, 90%, 95%, 96%, 97%, 98%, or 99% identity to the amino acid sequence of SEQ ID NO: 3 and optionally has an IL2 signaling sequence at its N-terminus, and an N-terminal MVRS amino acid sequence.

In some embodiments, E includes the amino acid sequence in SEQ ID NO: 4. In some embodiments, E includes the amino acid sequence having at least 70%, 75%, 80%, 85%, 90%, 95%, 96%, 97%, 98%, or 99% identity to the amino acid sequence of SEQ ID NO: 4 and, optionally has an MVRS amino acid sequence at its N-terminus.

In some embodiments, E includes the amino acid sequence of SEQ ID NO: 5. In some embodiments, E includes the amino acid sequence having at least 70%, 75%, 80%, 85%, 90%, 95%, 96%, 97%, 98%, or 99% identity to the amino acid sequence of SEQ ID NO: 5 and, optionally has an IL2 signaling sequence at its N-terminus and six histidine tags at its C-terminus.

In some embodiments, E includes the amino acid sequence in SEQ ID NO: 6. In some embodiments, E includes the amino acid sequence having at least 70%, 75%, 80%, 85%, 90%, 95%, 96%, 97%, 98%, or 99% identity to the amino acid sequence of SEQ ID NO: 6 and, optionally has six histidine tags at its C-terminus.

In some embodiments, E includes the amino acid sequence of SEQ ID NO: 7. In some embodiments, E includes the amino acid sequence having at least 70%, 75%, 80%, 85%, 90%, 95%, 96%, 97%, 98%, or 99% identity to the amino acid sequence of SEQ ID NO: 7 and, optionally, has an IL2 signaling sequence and a MVRS amino acid sequence at its N-terminus and six histidine tags at its C-terminus.

In some embodiments, E includes the amino acid sequence in SEQ ID NO: 8. In some embodiments, E includes the amino acid sequence having at least 70%, 75%, 80%, 85%, 90%, 95%, 96%, 97%, 98%, or 99% identity to the amino acid sequence of SEQ ID NO: 8 and, optionally, has an a MVRS amino acid sequence at its N-terminus and six histidine tags at its C-terminus.

In some embodiments, E includes the amino acid sequence of SEQ ID NO: 9. In some embodiments, E includes the amino acid sequence having at least 70%, 75%, 80%, 85%, 90%, 95%, 96%, 97%, 98%, or 99% identity to the amino acid sequence of SEQ ID NO: 9 and, optionally has an IL2 signaling sequence and an MVRS amino acid sequence at its N-terminus, two additional cysteines (at the position corresponding to position * of the SEQ ID NO:9) in its hinge region and six histidine tags at its C-terminus.

In some embodiments, E includes the amino acid sequence in SEQ ID NO: 10. In some embodiments, E includes the amino acid sequence having at least 70%, 75%, 80%, 85%, 90%, 95%, 96%, 97%, 98%, or 99% identity to the amino acid sequence of SEQ ID NO: 10 and, optionally has an MVRS amino acid sequence at its N-terminus, two additional cysteines (at the position corresponding to position * of the SEQ ID NO: 10) in its hinge region and six histidine tags at its C-terminus.

In some embodiments, E includes the amino acid sequence of SEQ ID NO: 11. In some embodiments, E includes the amino acid sequence having at least 70%, 75%, 80%, 85%, 90%, 95%, 96%, 97%, 98%, or 99% identity to the amino acid sequence of SEQ ID NO: 11 and, optionally has an MVRS amino acid sequence at its N-terminus, and two additional cysteines (at the position corresponding to position * of the SEQ ID NO:11) in its hinge region.

In some embodiments, E includes the amino acid sequence in SEQ ID NO: 12. In some embodiments, E includes the amino acid sequence having at least 70%, 75%, 80%, 85%, 90%, 95%, 96%, 97%, 98%, or 99% identity to the amino acid sequence of SEQ ID NO: 12 and, optionally has an IL2 signaling sequence at its N-terminus, an Asn to Ala substitution (at the position corresponding to position * of the SEQ ID NO:12), and six histidine tags at its C-terminus.

In some embodiments, E includes the amino acid sequence of SEQ ID NO: 13. In some embodiments, E includes the amino acid sequence having at least 70%, 75%, 80%, 85%, 90%, 95%, 96%, 97%, 98%, or 99% identity to the amino acid sequence of SEQ ID NO: 13 and, optionally has an Asn to Ala substitution (at the position corresponding to position * of the SEQ ID NO:13) and six histidine tags at its C-terminus.

In some embodiments, E includes the amino acid sequence in SEQ ID NO: 14. In some embodiments, E includes the amino acid sequence having at least 70%, 75%, 80%, 85%, 90%, 95%, 96%, 97%, 98%, or 99% identity to the amino acid sequence of SEQ ID NO: 14 and, optionally has an IL2 signaling sequence and an MVRS amino acid sequence at its N-terminus, an Asn to Ala substitution (at the position corresponding to position * of SEQ ID NO: 14) and six histidine tags at its C-terminus.

In some embodiments, E includes the amino acid sequence of SEQ ID NO: 15. In some embodiments, E includes the amino acid sequence having at least 70%, 75%, 80%, 85%, 90%, 95%, 96%, 97%, 98%, or 99% identity to the amino acid sequence of SEQ ID NO: 15 and, optionally has a MVRS amino acid sequence at its N-terminus, an Asn to Ala substitution (at the position corresponding to position * of SEQ ID NO: 15) and six histidine tags at its C-terminus.

In some embodiments, E includes the amino acid sequence in SEQ ID NO: 16. In some embodiments, E includes the amino acid sequence having at least 70%, 75%, 80%, 85%, 90%, 95%, 96%, 97%, 98%, or 99% identity to the amino acid sequence of SEQ ID NO: 16 and, optionally has a human serum albumin signaling sequence and an ISAMVRS amino acid sequence at its N-terminus.

In some embodiments, E includes the amino acid sequence of SEQ ID NO: 17. In some embodiments, E includes the amino acid sequence having at least 70%, 75%, 80%, 85%, 90%, 95%, 96%, 97%, 98%, or 99% identity to the amino acid sequence of SEQ ID NO: 17 and, optionally has a human serum albumin signaling sequence and an ISAMVRS amino acid sequence at its N-terminus, and a C-terminal G4S connector and a C-terminal c-Myc tag.

In some embodiments, E includes the amino acid sequence in SEQ ID NO: 18. In some embodiments, E includes the amino acid sequence having at least 70%, 75%, 80%, 85%, 90%, 95%, 96%, 97%, 98%, or 99% identity to the amino acid sequence of SEQ ID NO: 18 and, optionally has an ISAMVRS amino acid sequence at its N-terminus, a C-terminal G4S connector and a C-terminal c-Myc tag.

In some embodiments, E includes the amino acid sequence of SEQ ID NO: 19. In some embodiments, E includes the amino acid sequence having at least 70%, 75%, 80%, 85%, 90%, 95%, 96%, 97%, 98%, or 99% identity to the amino acid sequence of SEQ ID NO: 19 and, optionally, has an a human serum albumin signaling sequence and an ISAMVRS amino acid sequence at its N-terminus and contains lysine to serine mutation (at the position corresponding to position * of SEQ ID NO:19) to prevent coupling at this site.

In some embodiments, E includes the amino acid sequence in SEQ ID NO: 20. In some embodiments, E includes the amino acid sequence having at least 70%, 75%, 80%, 85%, 90%, 95%, 96%, 97%, 98%, or 99% identity to the amino acid sequence of SEQ ID NO: 20 and, optionally has an ISAMVRS amino acid sequence at the N-terminus and contains a lysine to serine mutation (at the position corresponding to position * of SEQ ID NO: 20) to prevent coupling at this site.

In some embodiments, E includes the amino acid sequence of SEQ ID NO: 21. In some embodiments, E includes the amino acid sequence having at least 70%, 75%, 80%, 85%, 90%, 95%, 96%, 97%, 98%, or 99% identity to the amino acid sequence of SEQ ID NO: 21, and, optionally has a human serum albumin signaling sequence and an ISAMVRS amino acid sequence at its N-terminus, an alteration of lysine to serine (at the position corresponding to position * of SEQ ID NO:21) to prevent coupling of this site, and contains a G4S connector and a c-Myc tag at its C-terminus.

In some embodiments, E includes the amino acid sequence in SEQ ID NO: 22. In some embodiments, E includes the amino acid sequence having at least 70%, 75%, 80%, 85%, 90%, 95%, 96%, 97%, 98%, or 99% identity to the amino acid sequence of SEQ ID NO: 22 and, optionally has an ISAMVRS amino acid sequence at its N-terminus, a lysine to serine alteration (at the position corresponding to position * of SEQ ID NO:22) to prevent coupling of this site, and a G4S connector and a c-Myc tag at its C-terminus.

In some embodiments, E includes the amino acid sequence of SEQ ID NO: 23. In some embodiments, E includes the amino acid sequence having at least 70%, 75%, 80%, 85%, 90%, 95%, 96%, 97%, 98%, or 99% identity to the amino acid sequence of SEQ ID NO: 23, and, optionally has an a human serum albumin signaling sequence and an ISAMVRS amino acid sequence at its N-terminus, an Asn to Ala alteration (at the position corresponding to position * of SEQ ID NO:23), and a G4S connector and a c-Myc tag at its C-terminus.

In some embodiments, E includes the amino acid sequence in SEQ ID NO: 24. In some embodiments, E includes the amino acid sequence having at least 70%, 75%, 80%, 85%, 90%, 95%, 96%, 97%, 98%, or 99% identity to the amino acid sequence of SEQ ID NO: 24 and, optionally has an ISAMVRS amino acid sequence at its N-terminus, an Asn to Ala alteration (at the position corresponding to position * of SEQ ID NO. 24), and a G4S connector and a c-Myc tag at its C-terminus.

In some embodiments, E includes the amino acid sequence of SEQ ID NO: 25. In some embodiments, E includes the amino acid sequence having at least 70%, 75%, 80%, 85%, 90%, 95%, 96%, 97%, 98%, or 99% identity to the amino acid sequence of SEQ ID NO: 25 and, optionally has a human serum albumin signaling sequence and an ISAMVRS amino acid sequence at its N-terminus, the H310A and H435A alterations to prevent FcRn binding, and the G4S connector and c-Myc tag at its C-terminus.

In some embodiments, E includes the amino acid sequence in SEQ ID NO: 26. In some embodiments, E includes the amino acid sequence having at least 70%, 75%, 80%, 85%, 90%, 95%, 96%, 97%, 98%, or 99% identity to the amino acid sequence of SEQ ID NO: 26 and, optionally has an ISAMVRS amino acid sequence at its N-terminus, the H310A and H435A alterations to prevent FcRn binding, and the G4S connector and c-Myc tag at its C-terminus.

In some embodiments, E includes the amino acid sequence of SEQ ID NO: 27. In some embodiments, E includes the amino acid sequence having at least 70%, 75%, 80%, 85%, 90%, 95%, 96%, 97%, 98%, or 99% identity to the amino acid sequence of SEQ ID NO:27 and, optionally has a human serum albumin signaling sequence and an ISAMVRS amino acid sequence at its N-terminus, and a G4S connector and a mutation (lysine to phenylalanine, at the position corresponding to the bold position of SEQ ID NO:27) and a c-Myc tag at its C-terminus.

In some embodiments, E includes the amino acid sequence in SEQ ID NO: 28. In some embodiments, E includes the amino acid sequence having at least 70%, 75%, 80%, 85%, 90%, 95%, 96%, 97%, 98%, or 99% identity to the amino acid sequence of SEQ ID NO: 28 and, optionally has an ISAMVRS amino acid sequence at its N-terminus, and a G4S connector and a mutation (lysine to phenylalanine, at the position corresponding to the bold position of SEQ ID NO:28) and a c-Myc tag at its C-terminus.

In some embodiments, E includes the amino acid sequence of SEQ ID NO: 29. In some embodiments, E includes the amino acid sequence having at least 70%, 75%, 80%, 85%, 90%, 95%, 96%, 97%, 98%, or 99% identity to the amino acid sequence of SEQ ID NO: 29 and, optionally has a human serum albumin signaling sequence and an ISAMVRS amino acid sequence at its N-terminus, an Asn to Ala substitution, and a G4S connector and a mutation (lysine to phenylalanine, at the position corresponding to the bold position of SEQ ID NO:29) and a c-Myc tag at its C-terminus.

In some embodiments, E includes the amino acid sequence in SEQ ID NO: 30. In some embodiments, E includes the amino acid sequence having at least 70%, 75%, 80%, 85%, 90%, 95%, 96%, 97%, 98%, or 99% identity to the amino acid sequence of SEQ ID NO: 30 and, optionally has an ISAMVRS amino acid sequence at its N-terminus, an Asn to Ala substitution (at the position corresponding to the * position of SEQ ID NO. 30), and a G4S connector, and a mutation (lysine to phenylalanine, at the position corresponding to the bold position of SEQ ID NO:30) and a c-Myc tag at its C-terminus.

In some embodiments, E includes the amino acid sequence of SEQ ID NO: 31. In some embodiments, E includes the amino acid sequence having at least 70%, 75%, 80%, 85%, 90%, 95%, 96%, 97%, 98%, or 99% identity to the amino acid sequence of SEQ ID NO: 31 and, optionally has a human serum albumin signaling sequence at its N-terminus, a homozygous isoform G1m(fa), and a G4S connector and a mutation (lysine to phenylalanine, at the position corresponding to the bold position of SEQ ID NO:31) and a c-Myc tag at its C-terminus.

In some embodiments, E includes the amino acid sequence in SEQ ID NO: 32. In some embodiments, E includes the amino acid sequence having at least 70%, 75%, 80%, 85%, 90%, 95%, 96%, 97%, 98%, or 99% identity to the amino acid sequence of SEQ ID NO: 32 and, optionally has a human serum albumin signal sequence at its N-terminus and an allotype G1m(fa).

In some embodiments, E includes the amino acid sequence in SEQ ID NO: 33. In some embodiments, E includes the amino acid sequence having at least 70%, 75%, 80%, 85%, 90%, 95%, 96%, 97%, 98%, or 99% identity to the amino acid sequence of SEQ ID NO: 33 and, optionally has an MVRS amino acid sequence at its N-terminus and a triple mutation of YTE.

In some embodiments, E includes the amino acid sequence of SEQ ID NO: 34. In some embodiments, E includes the amino acid sequence having at least 70%, 75%, 80%, 85%, 90%, 95%, 96%, 97%, 98%, or 99% identity to the amino acid sequence of SEQ ID NO:34 and, optionally has a human serum albumin signaling sequence, a hinge region EPKSS amino acid sequence of mature human Fc-IgG1 sequence, and a cysteine to serine alteration (at the position corresponding to position #of SEQ ID NO:34) at its N-terminus and contains an allotype G1m(fa).

In some embodiments, E includes the amino acid sequence in SEQ ID NO: 35. In some embodiments, E includes the amino acid sequence having at least 70%, 75%, 80%, 85%, 90%, 95%, 96%, 97%, 98%, or 99% identity to the amino acid sequence of SEQ ID NO: 35 and, optionally has its N-terminus containing a murine IgG signaling sequence and deleting the EPKSSD amino acid sequence in the hinge region of mature human Fc-IgG, and has an allotype G1m(fa).

In some embodiments, E includes the amino acid sequence in SEQ ID NO: 36. In some embodiments, E includes the amino acid sequence having at least 70%, 75%, 80%, 85%, 90%, 95%, 96%, 97%, 98%, or 99% identity to the amino acid sequence of SEQ ID NO: 36 and, optionally has deleted the EPKSSD amino acid sequence in the hinge region of mature human Fc-IgG at its N-terminus and contains an allotype G1m(fa) and a YTE triple mutation.

In some embodiments, E includes the amino acid sequence in SEQ ID NO: 37. In some embodiments, E includes the amino acid sequence having at least 70%, 75%, 80%, 85%, 90%, 95%, 96%, 97%, 98%, or 99% identity to the amino acid sequence of SEQ ID NO: 37 and, optionally has the deletion of the EPKSSD amino acid sequence in the hinge region of mature human Fc-IgG at its N-terminus and contains an LS double mutant and an allotype G1m(fa).

In some implementation schemes, E includes the amino acid sequence in SEQ ID NO: 38. In some implementation schemes, E includes the amino acid sequences having at least 70%, 75%, 80%, 85%, 90%, 95%, 96%, 97%, 98%, or 99% identity to the amino acid sequence of SEQ ID NO: 38, and optionally has a human serum albumin signal sequence at its N-terminus, a YTE triple mutation, and an allotype G1m (fa), and contains a G4S connector and a c-Myc tag at its C-terminus.

In some implementation schemes, E includes the amino acid sequence in SEQ ID NO: 39. In some implementation schemes, E includes the amino acid sequences having at least 70%, 75%, 80%, 85%, 90%, 95%, 96%, 97%, 98%, or 99% identity to the amino acid sequence of SEQ ID NO: 39, and optionally has the corresponding amino acids that are defined for position X of SEQ ID NO: 39.

In some embodiments, E includes the amino acid sequence of SEQ ID NO: 40. In some embodiments, E includes the amino acid sequence having at least 70%, 75%, 80%, 85%, 90%, 95%, 96%, 97%, 98%, or 99% identity to the amino acid sequence of SEQ ID NO: 40 and optionally has the corresponding amino acids that are defined for position X of SEQ ID NO: 40.

In some embodiments, E includes the amino acid sequence in SEQ ID NO: 41. In some embodiments, E includes the amino acid sequence having at least 70%, 75%, 80%, 85%, 90%, 95%, 96%, 97%, 98%, or 99% identity to the amino acid sequence of SEQ ID NO:41 and, optionally contains a YTE triple mutation and has the corresponding amino acids that are defined for position X of SEQ ID NO: 41.

In some embodiments, E includes the amino acid sequence of SEQ ID NO: 42. In some embodiments, E includes the amino acid sequence having at least 70%, 75%, 80%, 85%, 90%, 95%, 96%, 97%, 98%, or 99% identity to the amino acid sequence of SEQ ID NO: 42 and, optionally has a YTE triple mutation and an allotype G1m(fa).

In some embodiments, E includes the amino acid sequence in SEQ ID NO: 43. In some embodiments, E includes the amino acid sequence having at least 70%, 75%, 80%, 85%, 90%, 95%, 96%, 97%, 98%, or 99% identity to the amino acid sequence of SEQ ID NO: 43 and, optionally has a YTE triple mutation and an allotype G1m(f).

In some embodiments, E includes the amino acid sequence in SEQ ID NO: 44. In some embodiments, E includes the amino acid sequence having at least 70%, 75%, 80%, 85%, 90%, 95%, 96%, 97%, 98%, or 99% identity to the amino acid sequence of SEQ ID NO: 44 and, optionally, has an LS double mutant and the corresponding amino acids that are defined for position X of SEQ ID NO: 44 In some embodiments, E includes the amino acid sequence of SEQ ID NO: 45. In some embodiments, E includes the amino acid sequence having at least 70%, 75%, 80%, 85%, 90%, 95%, 96%, 97%, 98%, or 99% identity to the amino acid sequence of SEQ ID NO: 45 and, optionally has an LS double mutant and an allotype G1m(fa).

In some embodiments, E includes the amino acid sequence in SEQ ID NO: 46. In some embodiments, E includes the amino acid sequence having at least 70%, 75%, 80%, 85%, 90%, 95%, 96%, 97%, 98%, or 99% identity to the amino acid sequence of SEQ ID NO: 46 and, optionally has having an LS double mutant and an allotype G1m(f).

In some embodiments, E includes the amino acid sequence of SEQ ID NO: 47. In some embodiments, E includes the amino acid sequence having at least 70%, 75%, 80%, 85%, 90%, 95%, 96%, 97%, 98%, or 99% identity to the amino acid sequence of SEQ ID NO:47 and, optionally, has a mouse heavy chain MIgG Vh signaling sequence at its N-terminus, and a cysteine to serine alteration (at a position corresponding to position #of SEQ ID NO:47) and contains the corresponding amino acids that are defined for position X of SEQ ID NO: 47.

In some embodiments, E includes the amino acid sequence in SEQ ID NO: 48. In some embodiments, E includes the amino acid sequence having at least 70%, 75%, 80%, 85%, 90%, 95%, 96%, 97%, 98%, or 99% identity to the amino acid sequence of SEQ ID NO:48 and, optionally has a mouse heavy chain MIgG Vh signaling sequence at its N-terminus, and a cysteine to serine alteration (at a position corresponding to position #of SEQ ID NO:48) and contains an allotype G1m(fa).

In some embodiments, E includes the amino acid sequence in SEQ ID NO: 49. In some embodiments, E includes the amino acid sequence having at least 70%, 75%, 80%, 85%, 90%, 95%, 96%, 97%, 98%, or 99% identity to the amino acid sequence of SEQ ID NO:49 and, optionally has a mouse heavy chain MIgG Vh signaling sequence at its N-terminus, and a cysteine to serine alteration (at a position corresponding to position #of SEQ ID NO:49) and contains an allotype G1m(f).

In some embodiments, E includes the amino acid sequence in SEQ ID NO: 50. In some embodiments, E includes the amino acid sequence having at least 70%, 75%, 80%, 85%, 90%, 95%, 96%, 97%, 98%, or 99% identity to the amino acid sequence of SEQ ID NO:50 and, optionally has a mouse heavy chain MIgG Vh signaling sequence at its N-terminus, a cysteine to serine alteration (at a position corresponding to position #of SEQ ID NO:50), and mutants of M428L and N434S and contains an allotype G1m(fa).

In some embodiments, E includes the amino acid sequence in SEQ ID NO: 51. In some embodiments, E includes the amino acid sequence having at least 70%, 75%, 80%, 85%, 90%, 95%, 96%, 97%, 98%, or 99% identity to the amino acid sequence of SEQ ID NO:51 and, optionally has a mouse heavy chain MIgG Vh signaling sequence at its N-terminus, a cysteine to serine alteration (at a position corresponding to position #of SEQ ID NO:51), and mutants of M428L and N434S and contains an allotype G1m(f).

In some embodiments, E includes the amino acid sequence in SEQ ID NO: 52. In some embodiments, E includes the amino acid sequence having at least 70%, 75%, 80%, 85%, 90%, 95%, 96%, 97%, 98%, or 99% identity to the amino acid sequence of SEQ ID NO:52 and, optionally has a mouse heavy chain MIgG Vh signaling sequence at its N-terminus, a cysteine to serine alteration (at a position corresponding to position #of SEQ ID NO:52), and the YTE triple mutation and contains an allotype G1m(fa).

In some embodiments, E includes the amino acid sequence in SEQ ID NO: 53. In some embodiments, E includes the amino acid sequence having at least 70%, 75%, 80%, 85%, 90%, 95%, 96%, 97%, 98%, or 99% identity to the amino acid sequence of SEQ ID NO:53 and, optionally has a mouse heavy chain MIgG Vh signaling sequence at its N-terminus, a cysteine to serine alteration (at a position corresponding to position #of SEQ ID NO:53), and the YTE triple mutation and contains an allotype G1m(f).

In some embodiments, E includes the amino acid sequence in SEQ ID NO: 54. In some embodiments, E includes the amino acid sequence having at least 70%, 75%, 80%, 85%, 90%, 95%, 96%, 97%, 98%, or 99% identity to the amino acid sequence of SEQ ID NO:54 and, optionally has a mouse heavy chain MIgG Vh signaling sequence and an ISAMVRS amino sequence at its N-terminus, mutants of M428L and N434S, the G4S connector and c-Myc tag at its C-terminus, and contains the allotype G1m(f).

In some embodiments, E includes the amino acid sequence in SEQ ID NO: 55. In some embodiments, E includes the amino acid sequence having at least 70%, 75%, 80%, 85%, 90%, 95%, 96%, 97%, 98%, or 99% identity to the amino acid sequence of SEQ ID NO:55 and, optionally has a mouse heavy chain MIgG Vh signaling sequence and an ISAMVRS amino sequence at its N-terminus, mutants of M428L and N434S, the G4S connector and c-Myc tag at its C-terminus, and contains the allotype G1m(fa).

In some embodiments, E includes the amino acid sequence in SEQ ID NO: 56. In some embodiments, E includes the amino acid sequence having at least 70%, 75%, 80%, 85%, 90%, 95%, 96%, 97%, 98%, or 99% identity to the amino acid sequence of SEQ ID NO:56 and, optionally has a mouse heavy chain MIgG Vh signaling sequence and an ISAMVRS amino sequence at its N-terminus, YTE triple mutation, the G4S connector and c-Myc tag at its C-terminus, and contains the allotype G1m(f).

In some embodiments, E includes the amino acid sequence in SEQ ID NO: 57. In some embodiments, E includes the amino acid sequence having at least 70%, 75%, 80%, 85%, 90%, 95%, 96%, 97%, 98%, or 99% identity to the amino acid sequence of SEQ ID NO:57 and, optionally has a mouse heavy chain MIgG Vh signaling sequence and an ISAMVRS amino sequence at its N-terminus, YTE triple mutation, the G4S connector and c-Myc tag at its C-terminus, and contains the allotype G1m(fa).

In some embodiments, E includes the amino acid sequence in SEQ ID NO: 58. In some embodiments, E includes the amino acid sequence having at least 70%, 75%, 80%, 85%, 90%, 95%, 96%, 97%, 98%, or 99% identity to the amino acid sequence of SEQ ID NO:58 and, optionally has a mouse heavy chain MIgG Vh signaling sequence at its N-terminus, a cysteine to serine alteration (at a position corresponding to position #of SEQ ID NO:58), the G4S connector and IgA peptide tag at its C-terminus, and contains the allotype G1m(fa).

In some embodiments, E includes the amino acid sequence in SEQ ID NO: 59. In some embodiments, E includes the amino acid sequence having at least 70%, 75%, 80%, 85%, 90%, 95%, 96%, 97%, 98%, or 99% identity to the amino acid sequence of SEQ ID NO:59 and, optionally has a mouse heavy chain MIgG Vh signaling sequence at its N-terminus, a cysteine to serine alteration (at a position corresponding to position #of SEQ ID NO:59), mutants of M428L and N434S, the G4S connector and IgA peptide tag at its C-terminus, and contains the allotype G1m(fa).

In some embodiments, E includes the amino acid sequence of SEQ ID NO: 60. In some embodiments, E includes the amino acid sequence having at least 70%, 75%, 80%, 85%, 90%, 95%, 96%, 97%, 98%, or 99% identity to the amino acid sequence of SEQ ID NO:60 and, optionally contains the corresponding amino acids that are defined for positions X and Z of SEQ ID NO: 60.

In some embodiments, E includes the amino acid sequence of SEQ ID NO: 61. In some embodiments, E includes the amino acid sequence having at least 70%, 75%, 80%, 85%, 90%, 95%, 96%, 97%, 98%, or 99% identity to the amino acid sequence of SEQ ID NO:61 and, optionally has a cysteine to serine alteration (at a position corresponding to position #of SEQ ID NO:61), and contains the corresponding amino acids that are defined for position X of SEQ ID NO: 61.

In some embodiments, E includes the amino acid sequence of SEQ ID NO: 62. In some embodiments, E includes the amino acid sequence having at least 70%, 75%, 80%, 85%, 90%, 95%, 96%, 97%, 98%, or 99% identity to the amino acid sequence of SEQ ID NO:62 and, optionally has a cysteine to serine alteration (at a position corresponding to position #of SEQ ID NO:62), and contains the corresponding amino acids that are defined for position X of SEQ ID NO: 62.

In some embodiments, E includes the amino acid sequence of SEQ ID NO: 63. In some embodiments, E includes the amino acid sequence having at least 70%, 75%, 80%, 85%, 90%, 95%, 96%, 97%, 98%, or 99% identity to the amino acid sequence of SEQ ID NO:63 and, optionally has a cysteine to serine alteration (at a position corresponding to position #of SEQ ID NO:63), and contains an allotype G1m(f).

In some embodiments, E includes the amino acid sequence of SEQ ID NO: 64. In some embodiments, E includes the amino acid sequence having at least 70%, 75%, 80%, 85%, 90%, 95%, 96%, 97%, 98%, or 99% identity to the amino acid sequence of SEQ ID NO:64 and, optionally has a cysteine to serine alteration (at a position corresponding to position #of SEQ ID NO:64), and contains an allotype G1m(fa).

In some embodiments, E includes the amino acid sequence of SEQ ID NO: 65. In some embodiments, E includes the amino acid sequence having at least 70%, 75%, 80%, 85%, 90%, 95%, 96%, 97%, 98%, or 99% identity to the amino acid sequence of SEQ ID NO:65 and, optionally has a cysteine to serine alteration (at a position corresponding to position #of SEQ ID NO:65), mutants of M428L and N434S, and contains an allotype G1m(fa).

In some embodiments, E includes the amino acid sequence of SEQ ID NO: 66. In some embodiments, E includes the amino acid sequence having at least 70%, 75%, 80%, 85%, 90%, 95%, 96%, 97%, 98%, or 99% identity to the amino acid sequence of SEQ ID NO:66 and, optionally has a cysteine to serine alteration (at a position corresponding to position #of SEQ ID NO:66), mutants of M428L and N434S, and contains an allotype G1m(f).

In some embodiments, E includes the amino acid sequence of SEQ ID NO: 67. In some embodiments, E includes the amino acid sequence having at least 70%, 75%, 80%, 85%, 90%, 95%, 96%, 97%, 98%, or 99% identity to the amino acid sequence of SEQ ID NO:67 and, optionally has a cysteine to serine alteration (at a position corresponding to position #of SEQ ID NO:67), the YTE triple mutation, and contains an allotype G1m(fa).

In some embodiments, E includes the amino acid sequence of SEQ ID NO: 68. In some embodiments, E includes the amino acid sequence having at least 70%, 75%, 80%, 85%, 90%, 95%, 96%, 97%, 98%, or 99% identity to the amino acid sequence of SEQ ID NO:68 and, optionally has a cysteine to serine alteration (at a position corresponding to position #of SEQ ID NO:68), the YTE triple mutation, and contains an allotype G1m(f).

In any one of the embodiments described herein, E includes an Fc structural domain monomer, the Fc structural domain monomer (e.g., a sequence having any of SEQ ID NOs: 1-68) including a triple mutation corresponding to M252Y/S254T/T265E(YTE). Those skilled in the art will appreciate that “corresponding to” an amino acid site at a specific corresponding position obtained by sequence homology comparison using the sequence analysis software (e.g., but not limited to, DNA Star, Vector NTI, etc.). For example, any of SEQ ID NOs: 1-68 may be mutated to include a YTE mutation.

In any one of the embodiments described herein, E includes an Fc structural domain monomer, the Fc structural domain monomer (e.g., a sequence having any of SEQ ID NOs: 1-68) including a double mutant corresponding to M428L/N434S(LS). Those skilled in the art will appreciate that “corresponding to” an amino acid site at a specific corresponding position obtained by sequence homology comparison using the sequence analysis software (e.g., but not limited to, DNA Star, Vector NTI, etc.). For example, any of SEQ ID NOs: 1-68 may be mutated to include a LS mutation.

In any one of the embodiments described herein, E includes an Fc structural domain monomer, the Fc structural domain monomer (e.g., a sequence having any of SEQ ID NOs: 1-68) including a double mutant corresponding to N434H. Those skilled in the art will appreciate that “corresponding to” an amino acid site at a specific corresponding position obtained by sequence homology comparison using the sequence analysis software (e.g., but not limited to, DNA Star, Vector NTI, etc.). For example, any of SEQ ID NOs: 1-68 may be mutated to include an N434H mutation.

In any one of the embodiments described herein, E includes an Fc structural domain monomer, the Fc structural domain monomer (e.g., a sequence having any of SEQ ID NOs: 1-68) including a double mutant corresponding to C220S. Those skilled in the art will appreciate that “corresponding to” an amino acid site at a specific corresponding position obtained by sequence homology comparison using the sequence analysis software (e.g., but not limited to, DNA Star, Vector NTI, etc.). For example, any of SEQ TD NOs: 1-68 may be mutated to include a C220S mutation.

In any one of the embodiments described herein, E includes a fragment of an Fc structural domain monomer (e.g., a fragment of an Fc structural domain monomer from any sequence of SEQ ID NOs: 1-68) and is a fragment of at least 25 (e.g., 20, 21, 22. 23, 24, 25, 26, 27, 28, 29, 30, 31, 32, 33, 34, 35, 36, 37, 38, 39, 40, 41, 42, 43, 44, 45, 46, 47, 48, 49, 50 or more), or at least 50 (e.g., 51, 52, 53, 54, 55, 56, 57, 58, 59, 60, 61, 62, 63, 64, 65, 66, 67, 68, 69, 70, 71, 72, 73, 74, 75 or more) or at least 75 (75, 76, 77, 78, 79, 80, 81, 82, 83, 84, 85, 86, 87, 88, 89, 90, 91, 92, 93, 94, 95, 96, 97, 98, 99, 100 or more) consecutive amino acid in length from such Fc structural domain monomer (e.g., the Fc structural domain monomer from any sequence of SEQ ID NOs: 1-68).

In one embodiment, said conjugates have the following structures:

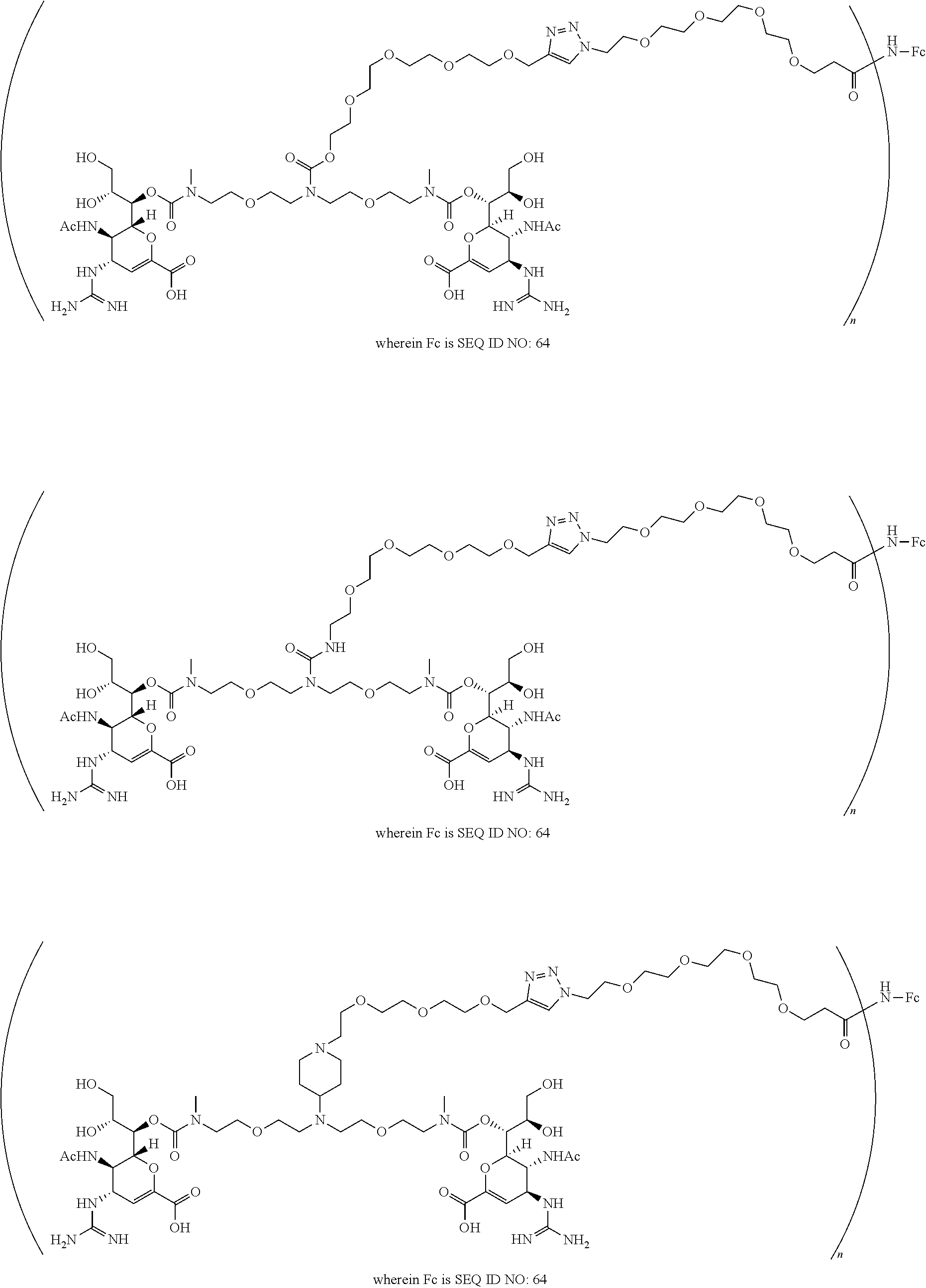

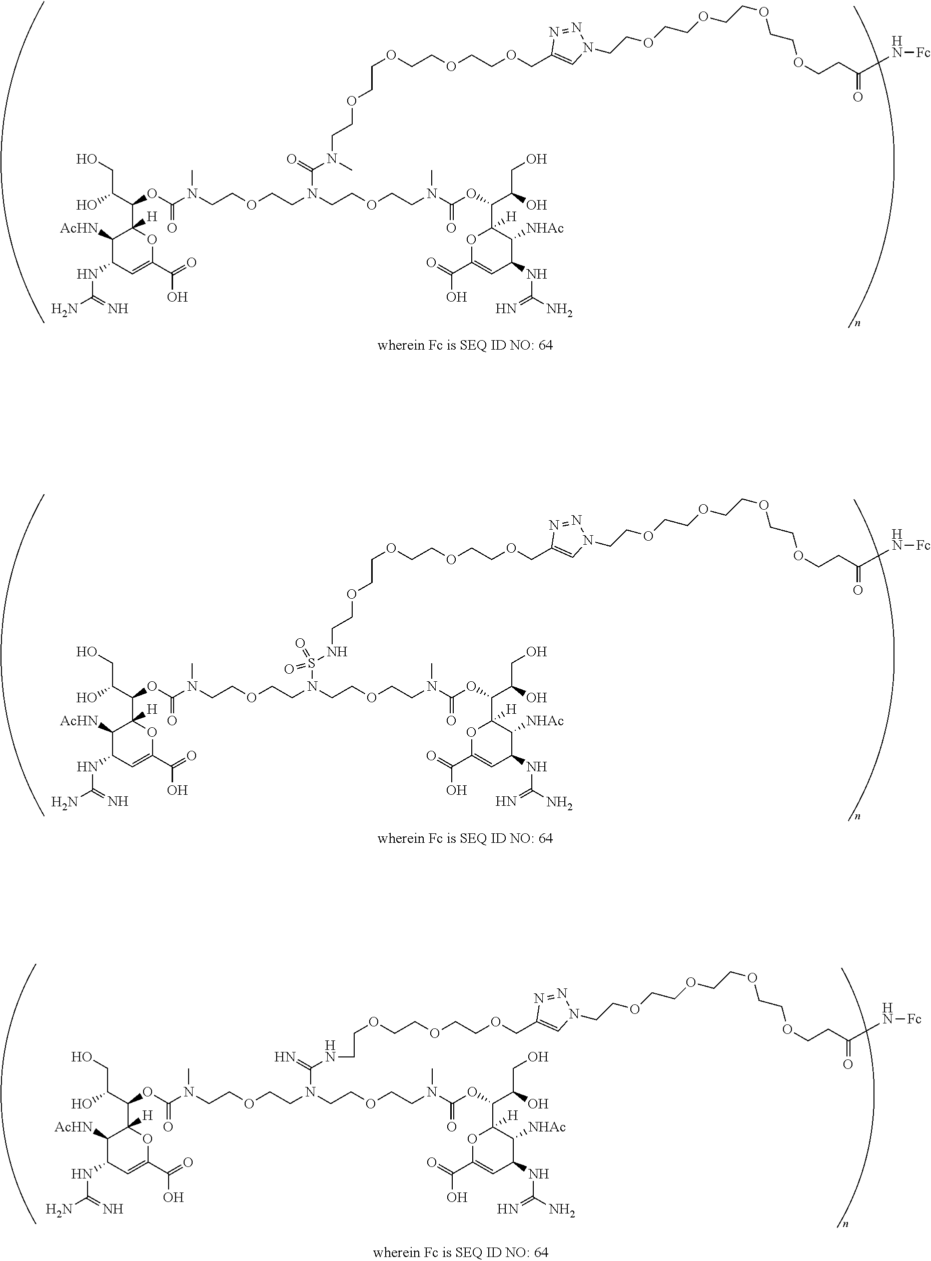

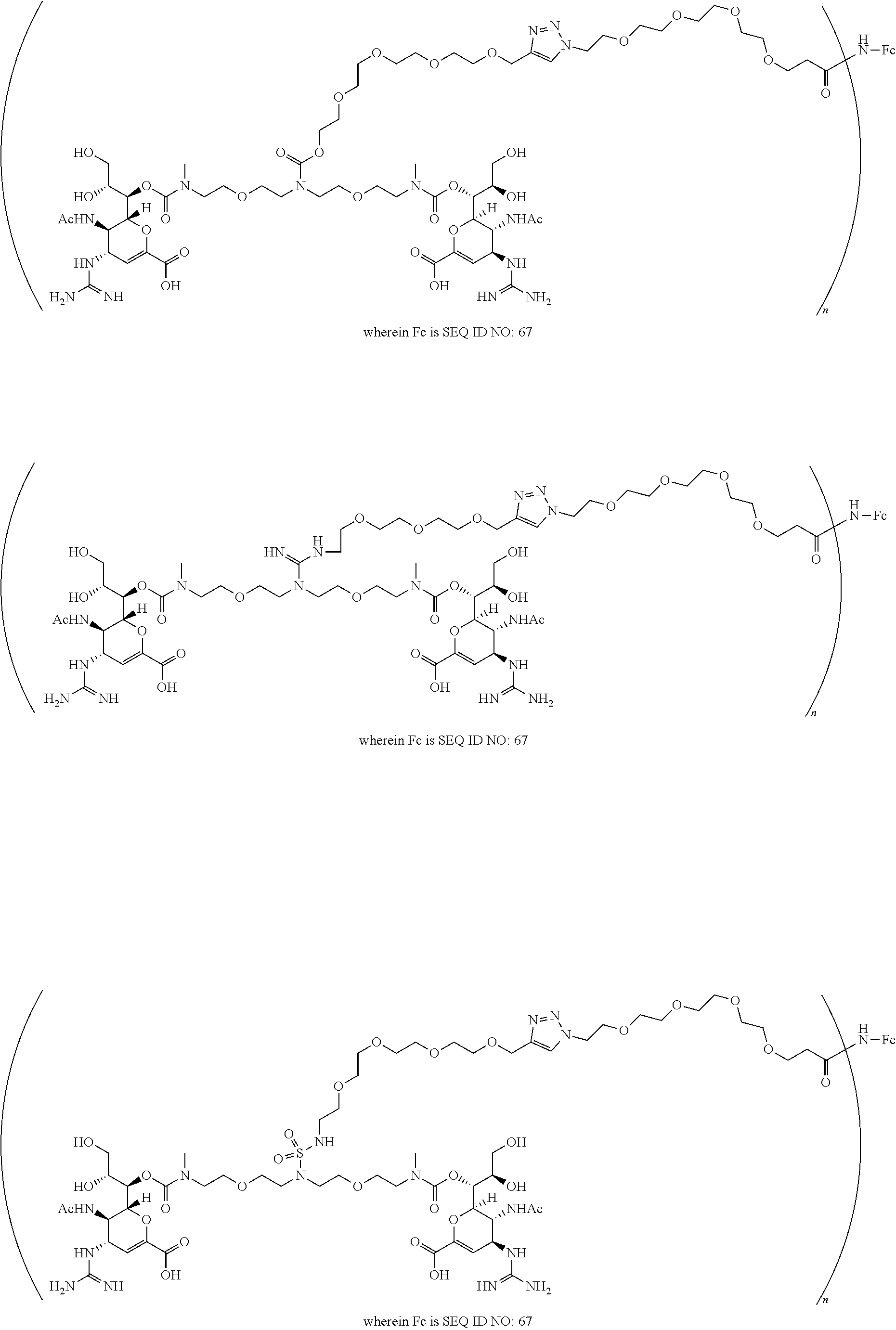

wherein, n is as defined above.

In one aspect, provided therein is a compound of formula I-2, or a pharmaceutically acceptable salt, ester, isomer, solvate, prodrug or isotopically labeled derivative thereof

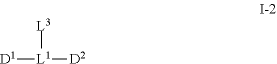

wherein L¹, D¹ and D² are as defined in above formula I-1;
L³ is selected from following structures:

| L³ structure | |
|---|---|
| No. | Structure |
| L3-1 | 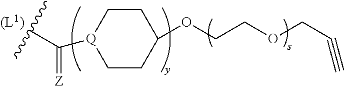 |
| L3-2 | 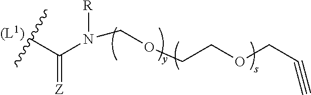 |
| L3-3 | 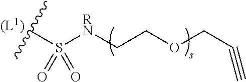 |
| L3-4 | 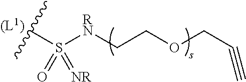 |
| L3-5 | 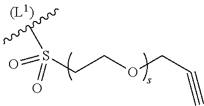 |
| L3-6 | 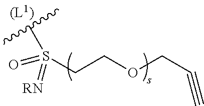 |
| L3-7 | 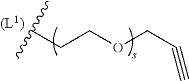 |
| L3-8 | 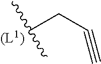 [SMILES: *CC#C] |
| L3-9 | 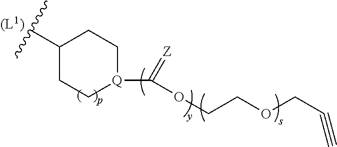 |
| L3-10 | 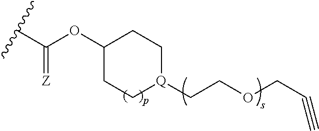 |
| L3-11 | 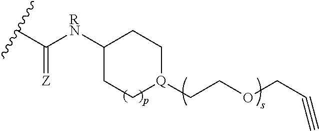 |
| L3-12 | 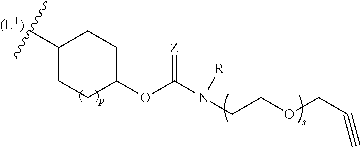 |
| L3-13 | 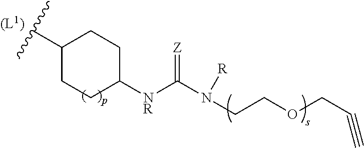 |
| L3-14 | 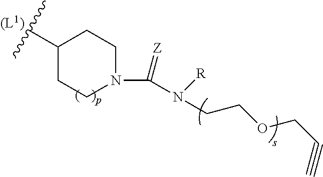 |
| L3-15 | 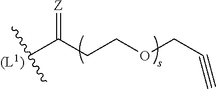 |

wherein each variable such as Z, R, Q, s, y, p is as defined above for L².

In some embodiments, compounds of formula I-2 preferably have the structure of Formula C-Inter-1 to C-Inter-115:

| No. | Structure |
|---|---|
| C- Inter- 1 | 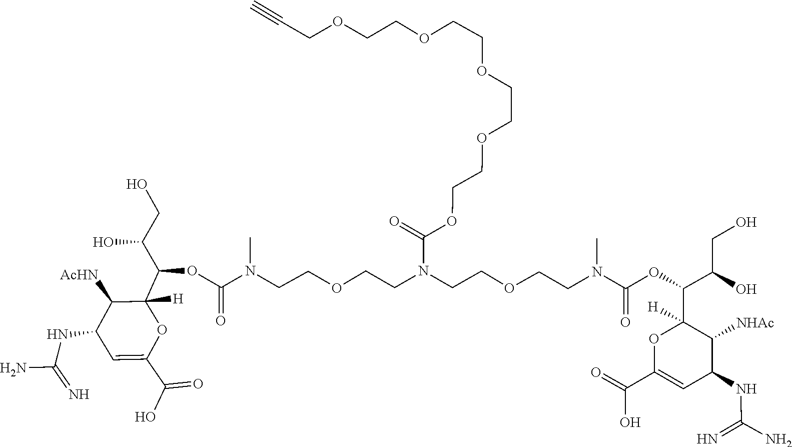 [SMILES: C#CCOCCOCCOCCOCCOC(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C- Inter- 2 | 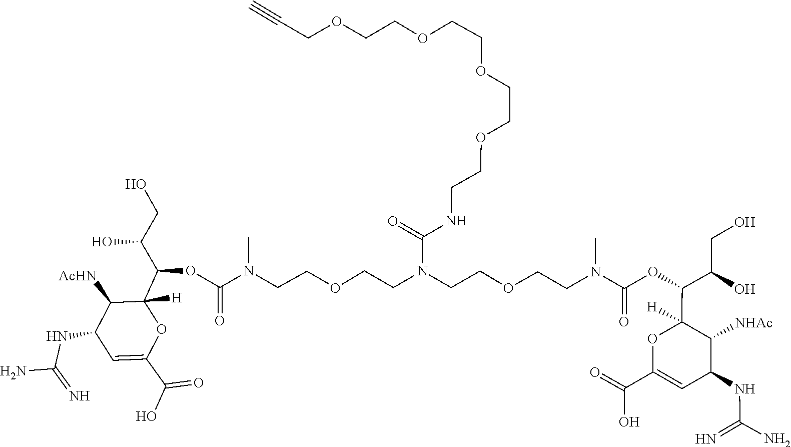 [SMILES: C#CCOCCOCCOCCOCCNC(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C- Inter- 3 | 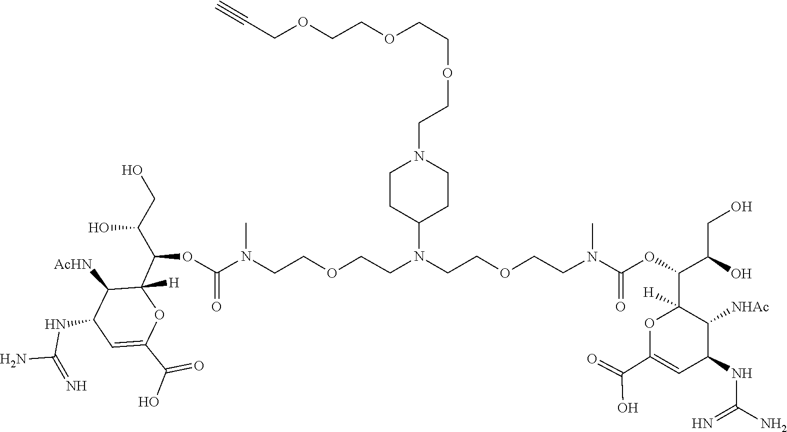 [SMILES: C#CCOCCOCCOCCN1CCC(N(CCOCCN(C)C(=O)O[C@@H]([C@@H]2OC(C(=O)O)=C[C@H](NC(=N)N)[C@H]2NC(C)=O)[C@H](O)CO)CCOCCN(C)C(=O)O[C@@H]([C@@H]2OC(C(=O)O)=C[C@H](NC(=N)N)[C@H]2NC(C)=O)[C@H](O)CO)CC1] |
| C- Inter- 4 | 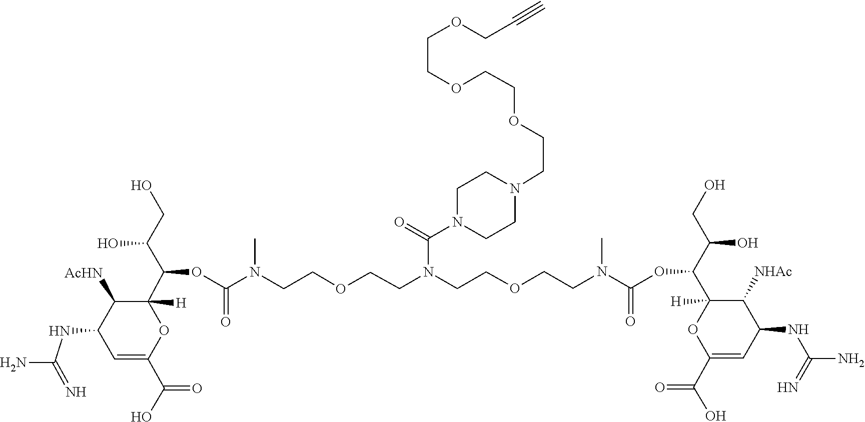 [SMILES: C#CCOCCOCCOCCN1CCN(C(=O)N(CCOCCN(C)C(=O)O[C@@H]([C@@H]2OC(C(=O)O)=C[C@H](NC(=N)N)[C@H]2NC(C)=O)[C@H](O)CO)CCOCCN(C)C(=O)O[C@@H]([C@@H]2OC(C(=O)O)=C[C@H](NC(=N)N)[C@H]2NC(C)=O)[C@H](O)CO)CC1] |
| C- Inter- 5 | 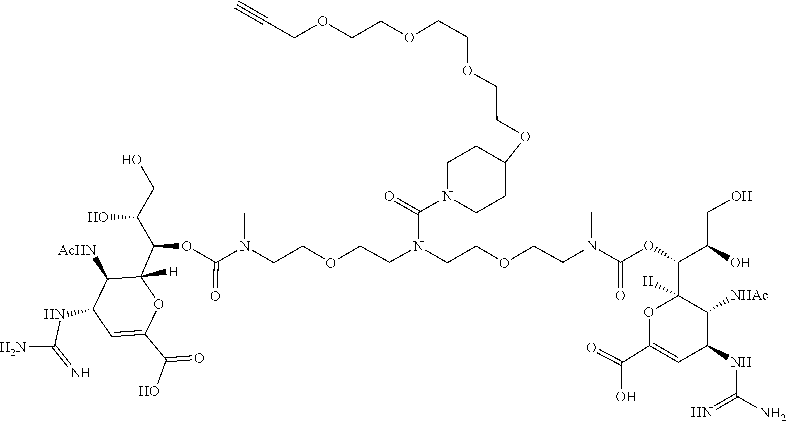 [SMILES: C#CCOCCOCCOCCOC1CCN(C(=O)N(CCOCCN(C)C(=O)O[C@@H]([C@@H]2OC(C(=O)O)=C[C@H](NC(=N)N)[C@H]2NC(C)=O)[C@H](O)CO)CCOCCN(C)C(=O)O[C@@H]([C@@H]2OC(C(=O)O)=C[C@H](NC(=N)N)[C@H]2NC(C)=O)[C@H](O)CO)CC1] |
| C- Inter- 6 | 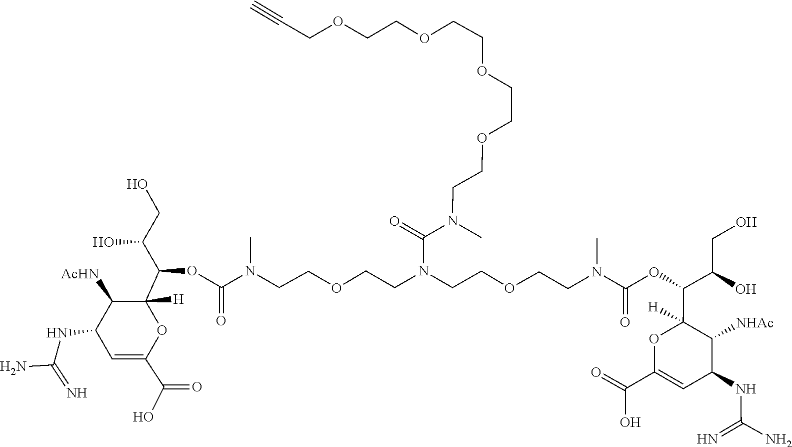 [SMILES: C#CCOCCOCCOCCOCCN(C)C(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C- Inter- 7 | 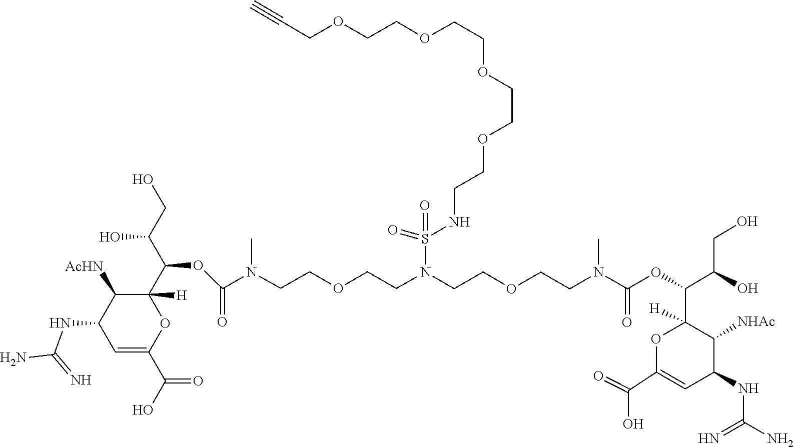 [SMILES: C#CCOCCOCCOCCOCCNS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C- Inter- 8 | 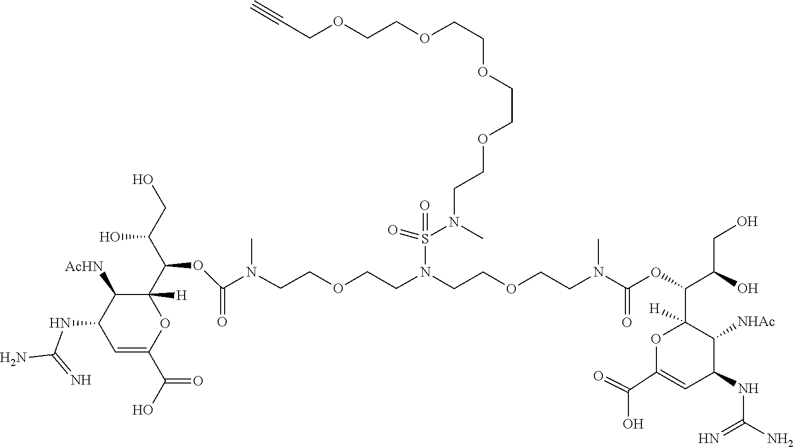 [SMILES: C#CCOCCOCCOCCOCCN(C)S(=O)(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C- Inter- 9 | 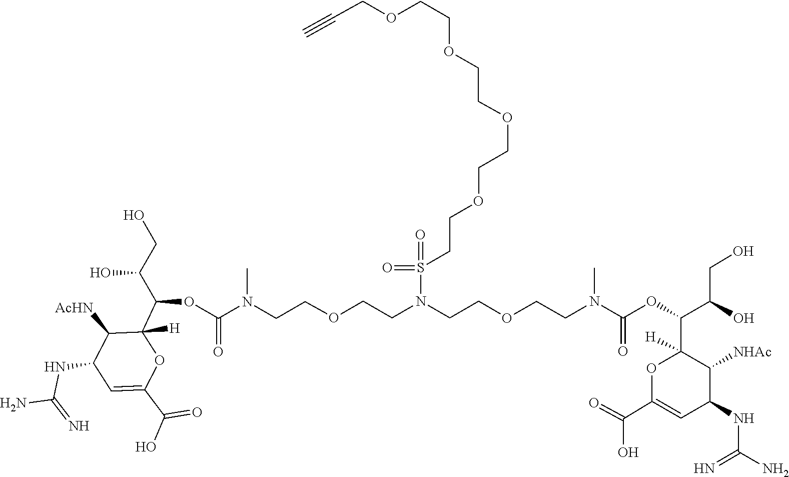 [SMILES: C#CCOCCOCCOCCOCCS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C- Inter- 10 | 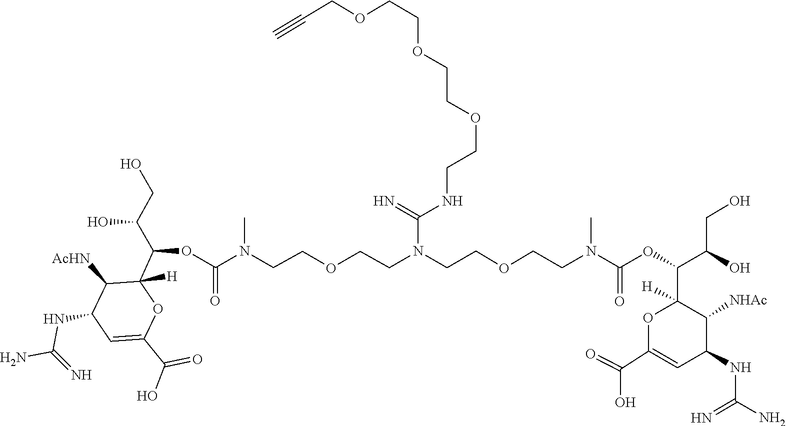 [SMILES: C#CCOCCOCCOCCNC(=N)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C- Inter- 11 | 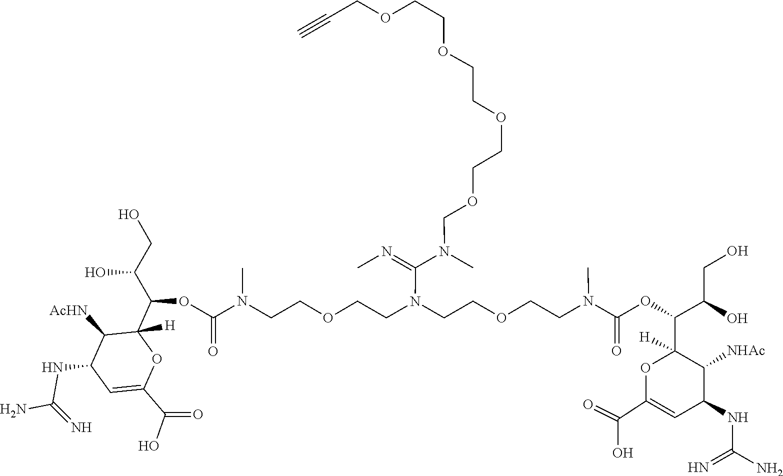 [SMILES: C#CCOCCOCCOCCOCN(C)/C(=N/C)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C- Inter- 12 | 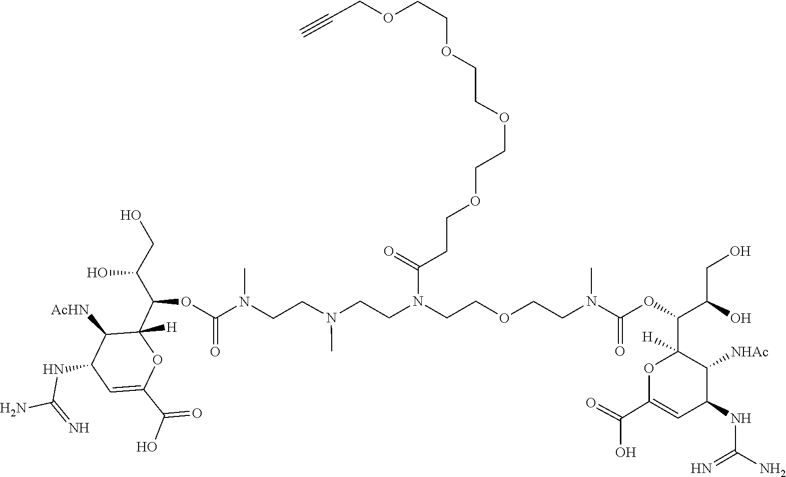 [SMILES: C#CCOCCOCCOCCOCCC(=O)N(CCOCCN(C)C(=O)O[C@@H]([C@@H]1OC(C(=O)O)=C[C@H](NC(=N)N)[C@H]1NC(C)=O)[C@H](O)CO)CCN(C)CCN(C)C(=O)O[C@@H]([C@@H]1OC(C(=O)O)=C[C@H](NC(=N)N)[C@H]1NC(C)=O)[C@H](O)CO] |
| C- Inter- 13 | 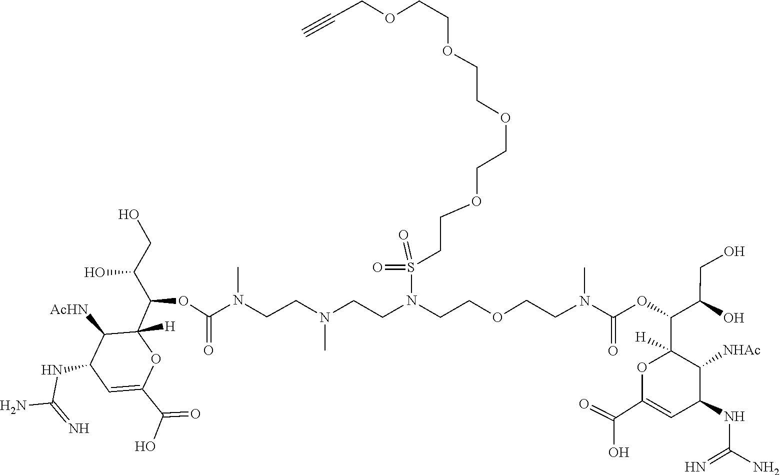 [SMILES: C#CCOCCOCCOCCOCCS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCN(C)CCN(C)C(=O)O[C@@H]([C@@H]1OC(C(=O)O)=C[C@H](NC(=N)N)[C@H]1NC(C)=O)[C@H](O)CO] |
| C- Inter- 14 | 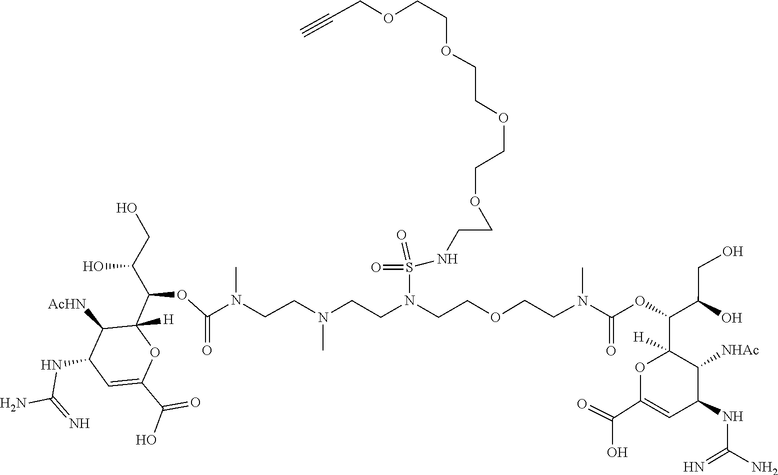 [SMILES: C#CCOCCOCCOCCOCCNS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCN(C)CCN(C)C(=O)O[C@@H]([C@@H]1OC(C(=O)O)=C[C@H](NC(=N)N)[C@H]1NC(C)=O)[C@H](O)CO] |
| C- Inter- 15 | 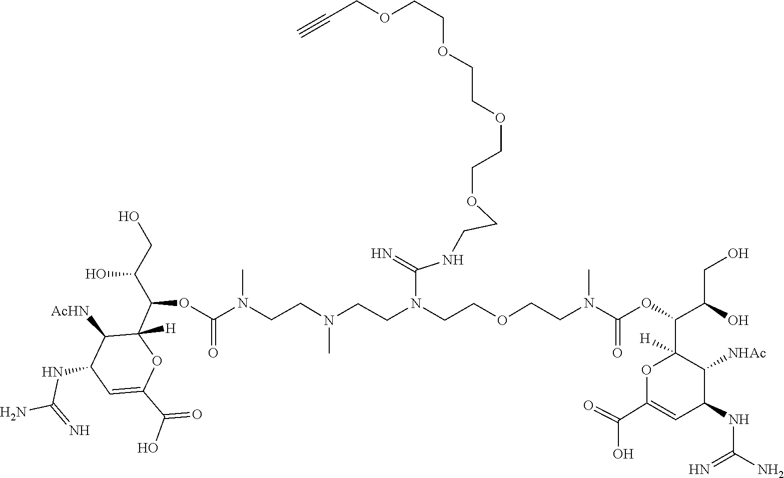 [SMILES: C#CCOCCOCCOCCOCCNC(=N)N(CCOCCN(C)C(=O)O[C@@H]([C@@H]1OC(C(=O)O)=C[C@H](NC(=N)N)[C@H]1NC(C)=O)[C@H](O)CO)CCN(C)CCN(C)C(=O)O[C@@H]([C@@H]1OC(C(=O)O)=C[C@H](NC(=N)N)[C@H]1NC(C)=O)[C@H](O)CO] |
| C- Inter- 16 | 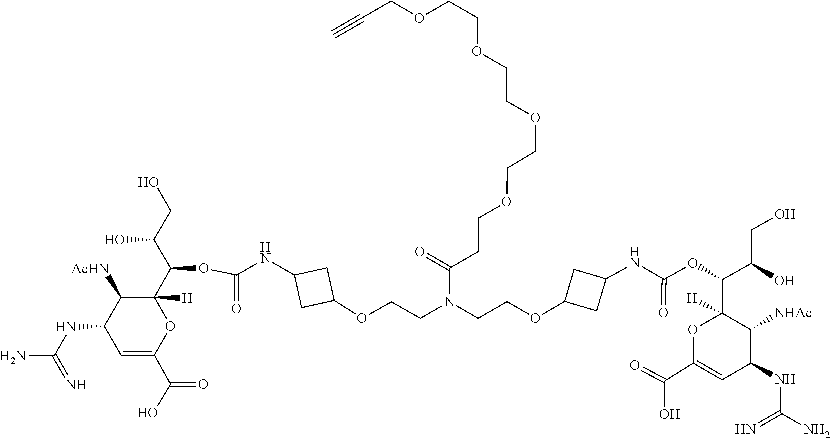 [SMILES: C#CCOCCOCCOCCOCCC(=O)N(CCOC1CC(NC(=O)O[C@@H]([C@@H]2OC(C(=O)O)=C[C@H](NC(=N)N)[C@H]2NC(C)=O)[C@H](O)CO)C1)CCOC1CC(NC(=O)O[C@@H]([C@@H]2OC(C(=O)O)=C[C@H](NC(=N)N)[C@H]2NC(C)=O)[C@H](O)CO)C1] |
| C- Inter- 17 | 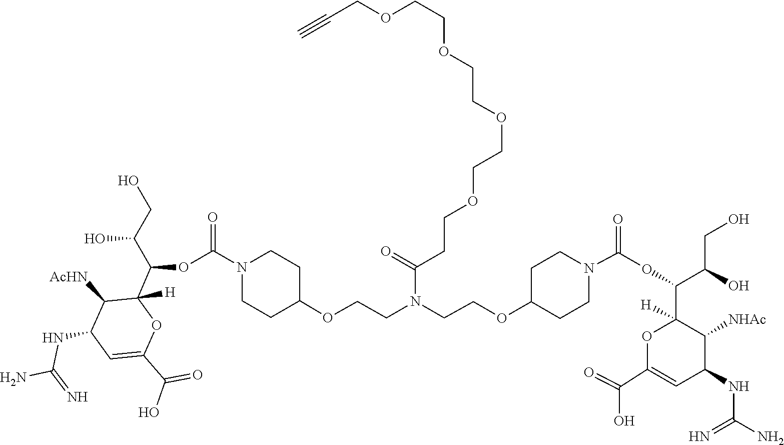 [SMILES: C#CCOCCOCCOCCOCCC(=O)N(CCOC1CCN(C(=O)O[C@@H]([C@@H]2OC(C(=O)O)=C[C@H](NC(=N)N)[C@H]2NC(C)=O)[C@H](O)CO)CC1)CCOC1CCN(C(=O)O[C@@H]([C@@H]2OC(C(=O)O)=C[C@H](NC(=N)N)[C@H]2NC(C)=O)[C@H](O)CO)CC1] |
| C- Inter- 18 | 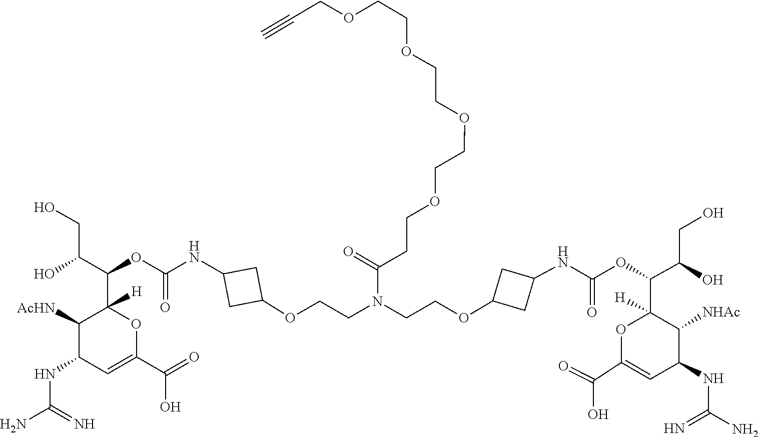 [SMILES: C#CCOCCOCCOCCOCCC(=O)N(CCOC1CC(NC(=O)O[C@@H]([C@@H]2OC(C(=O)O)=C[C@H](NC(=N)N)[C@H]2NC(C)=O)[C@H](O)CO)C1)CCOC1CC(NC(=O)O[C@@H]([C@@H]2OC(C(=O)O)=C[C@H](NC(=N)N)[C@H]2NC(C)=O)[C@H](O)CO)C1] |
| C- Inter- 19 | 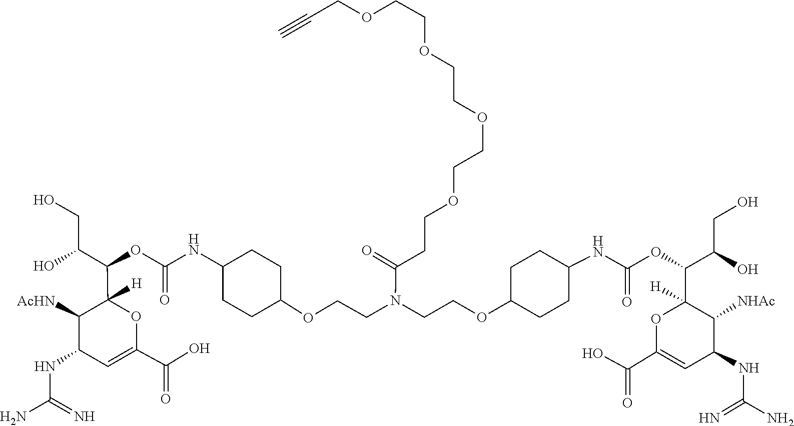 [SMILES: C#CCOCCOCCOCCOCCC(=O)N(CCOC1CCC(NC(=O)O[C@@H]([C@@H]2OC(C(=O)O)=C[C@H](NC(=N)N)[C@H]2NC(C)=O)[C@H](O)CO)CC1)CCOC1CCC(NC(=O)O[C@@H]([C@@H]2OC(C(=O)O)=C[C@H](NC(=N)N)[C@H]2NC(C)=O)[C@H](O)CO)CC1] |
| C- Inter- 20 | 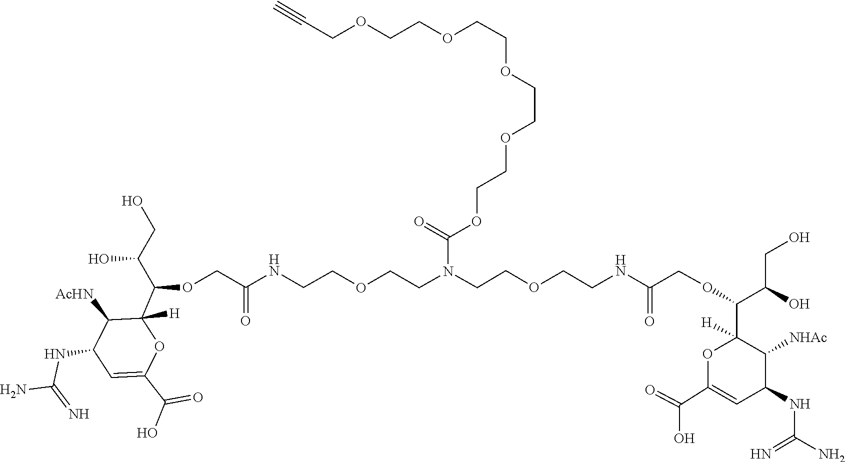 [SMILES: C#CCOCCOCCOCCOCCOC(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C- Inter- 21 | 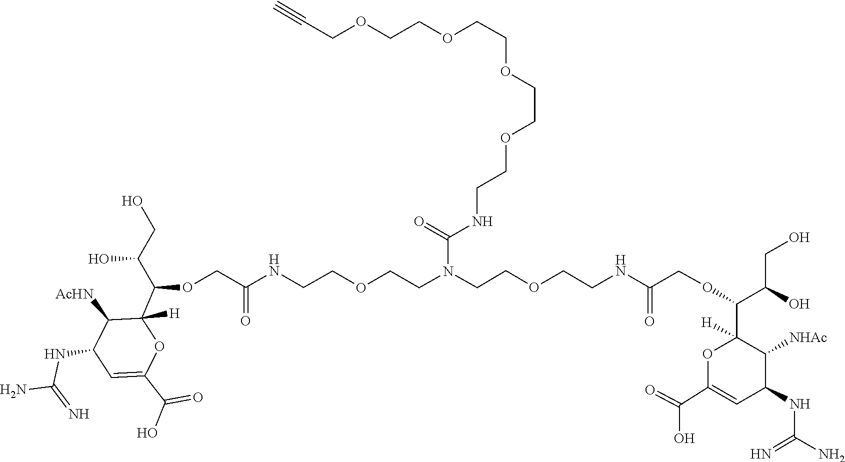 [SMILES: C#CCOCCOCCOCCOCCNC(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C- Inter- 22 | 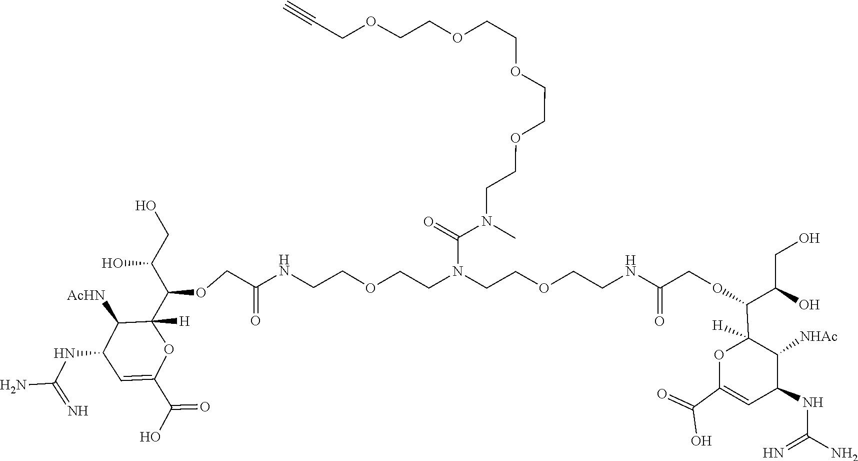 [SMILES: C#CCOCCOCCOCCOCCN(C)C(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C- Inter- 23 | 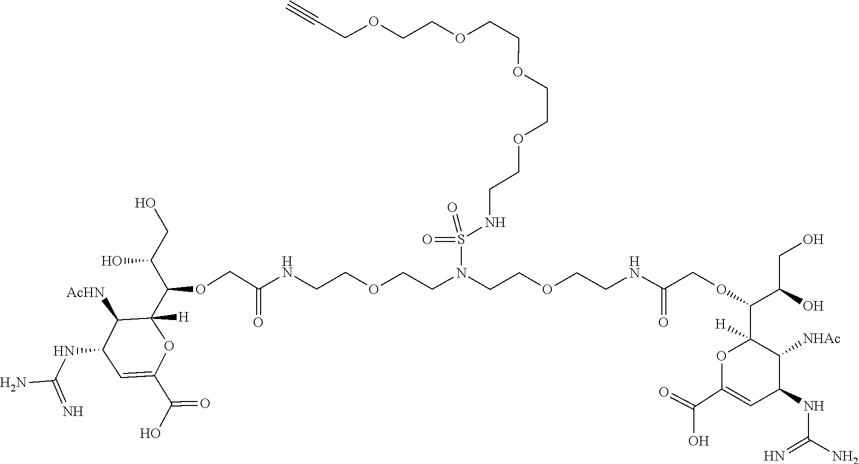 [SMILES: C#CCOCCOCCOCCOCCNS(=O)(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C- Inter- 24 | 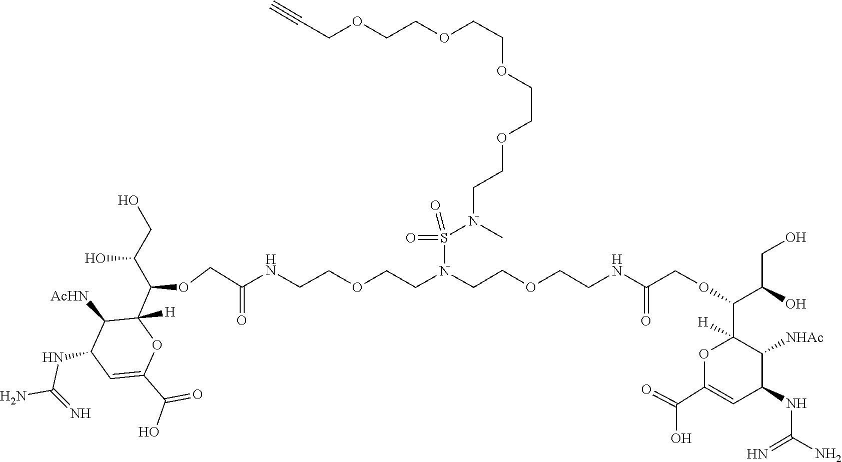 [SMILES: C#CCOCCOCCOCCOCCN(C)S(=O)(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C- Inter- 25 | 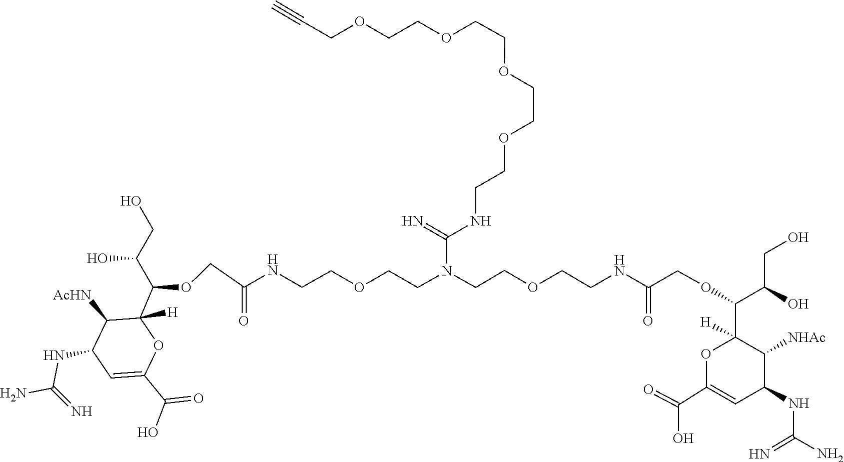 [SMILES: C#CCOCCOCCOCCOCCNC(=N)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C- Inter- 26 | 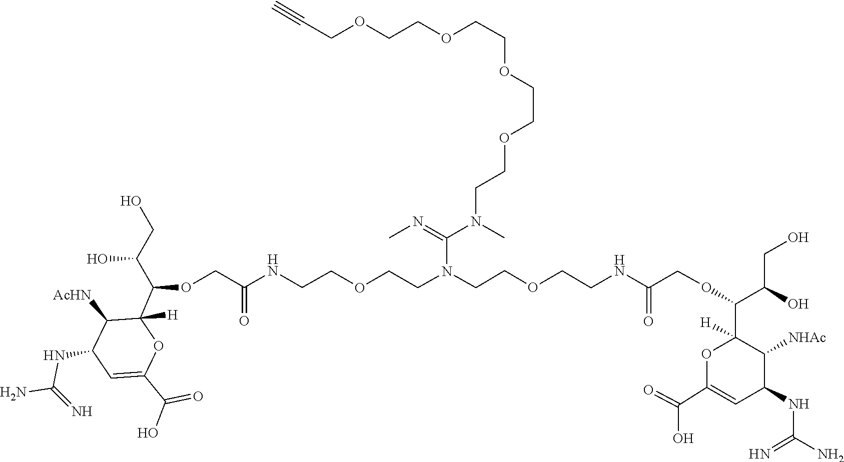 [SMILES: C#CCOCCOCCOCCOCCN(C)/C(=N/C)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C- Inter- 27 | 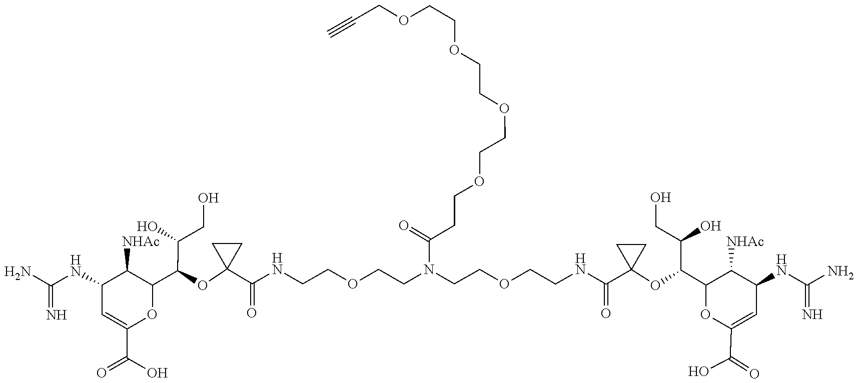 [SMILES: C#CCOCCOCCOCCOCCC(=O)N(CCOCCNC(=O)C1(O[C@@H](C2OC(C(=O)O)=C[C@H](NC(=N)N)[C@H]2NC(C)=O)[C@H](O)CO)CC1)CCOCCNC(=O)C1(O[C@@H](C2OC(C(=O)O)=C[C@H](NC(=N)N)[C@H]2NC(C)=O)[C@H](O)CO)CC1] |
| C- Inter- 28 | 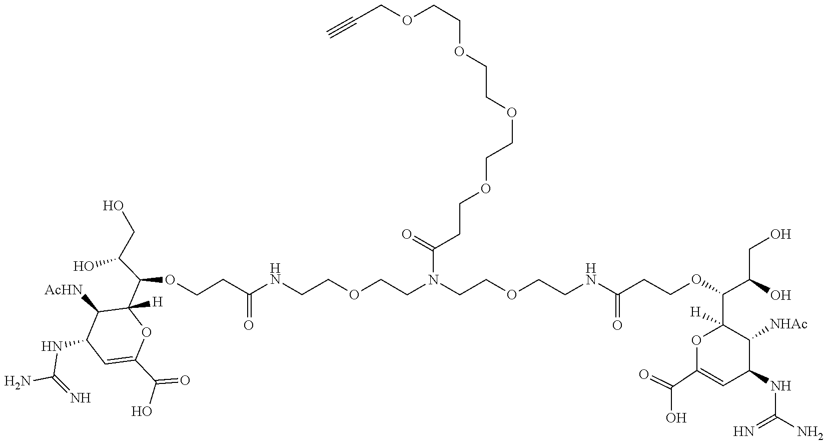 |
| C- Inter- 29 | 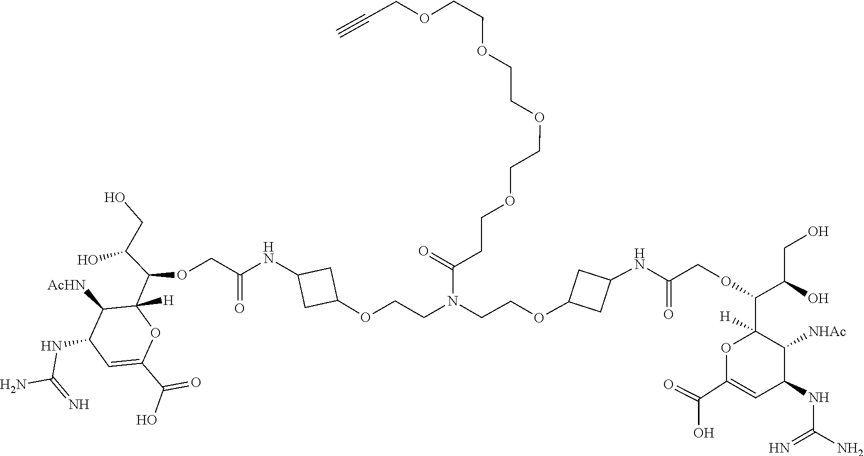 |
| C- Inter- 30 | 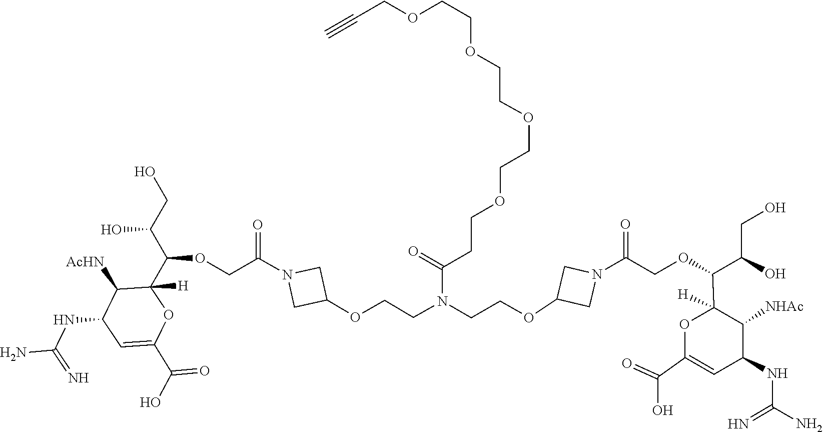 [SMILES: C#CCOCCOCCOCCOCCC(=O)N(CCOC1CN(C(=O)CO[C@@H]([C@@H]2OC(C(=O)O)=C[C@H](NC(=N)N)[C@H]2NC(C)=O)[C@H](O)CO)C1)CCOC1CN(C(=O)CO[C@@H]([C@@H]2OC(C(=O)O)=C[C@H](NC(=N)N)[C@H]2NC(C)=O)[C@H](O)CO)C1] |
| C- Inter- 31 | 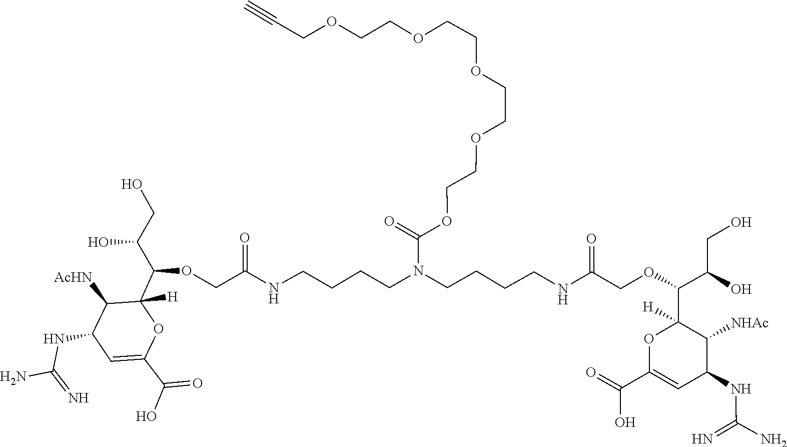 [SMILES: C#CCOCCOCCOCCOCCOC(=O)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| C- Inter- 32 | 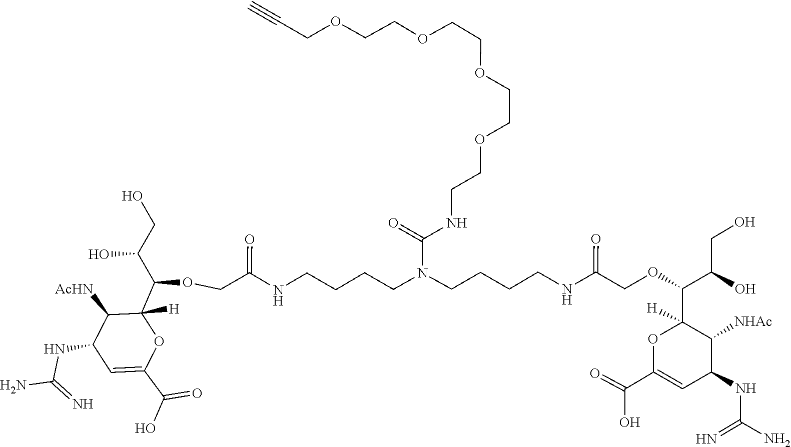 [SMILES: C#CCOCCOCCOCCOCCNC(=O)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| C- Inter- 33 | 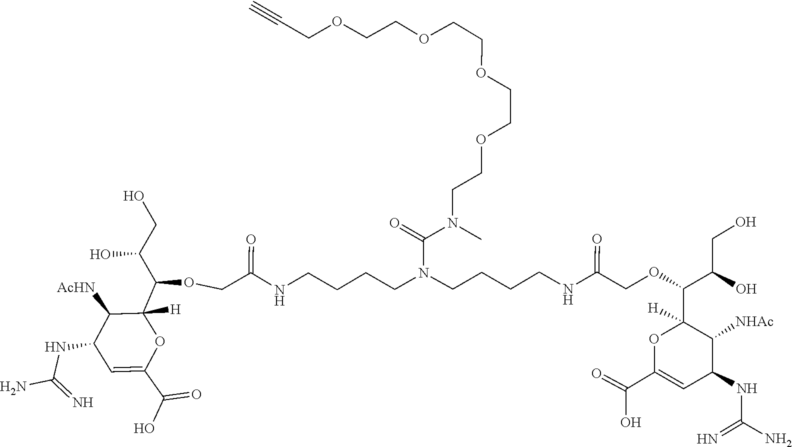 [SMILES: C#CCOCCOCCOCCOCCN(C)C(=O)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| C- Inter- 34 | 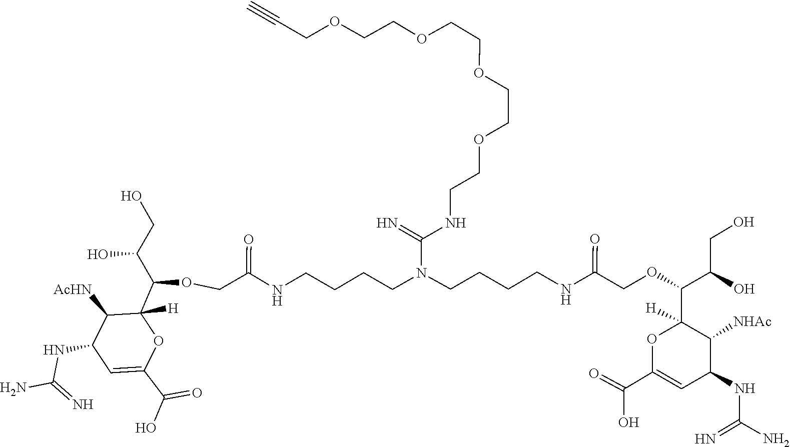 [SMILES: C#CCOCCOCCOCCOCCNC(=N)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| C- Inter- 35 | 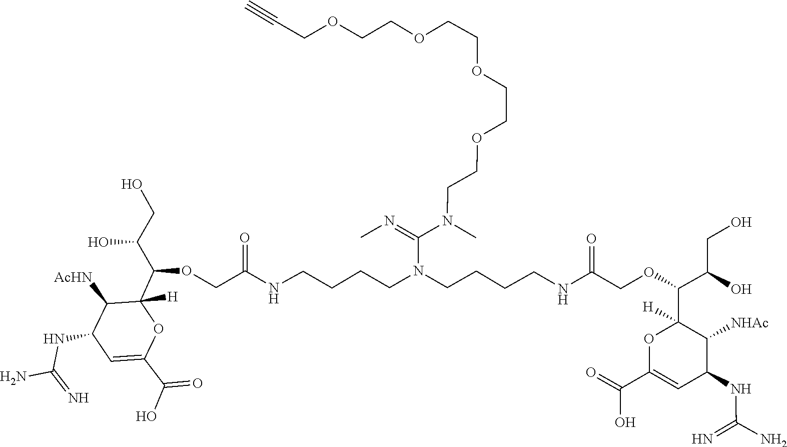 [SMILES: C#CCOCCOCCOCCOCCN(C)/C(=N/C)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| C- Inter- 36 | 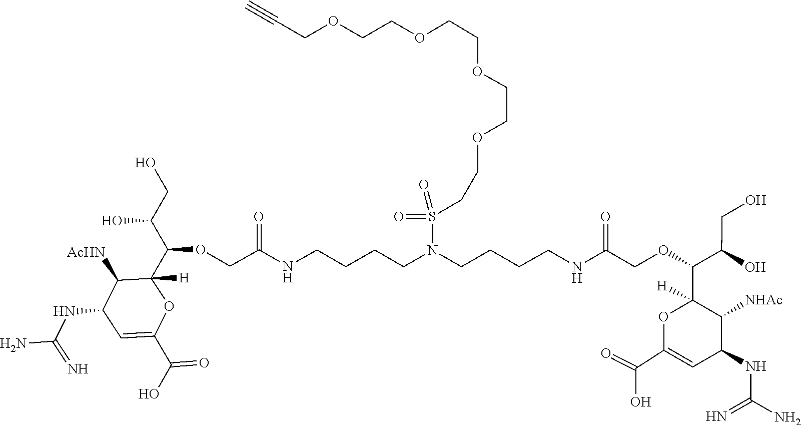 [SMILES: C#CCOCCOCCOCCOCCS(=O)(=O)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| C- Inter- 37 | 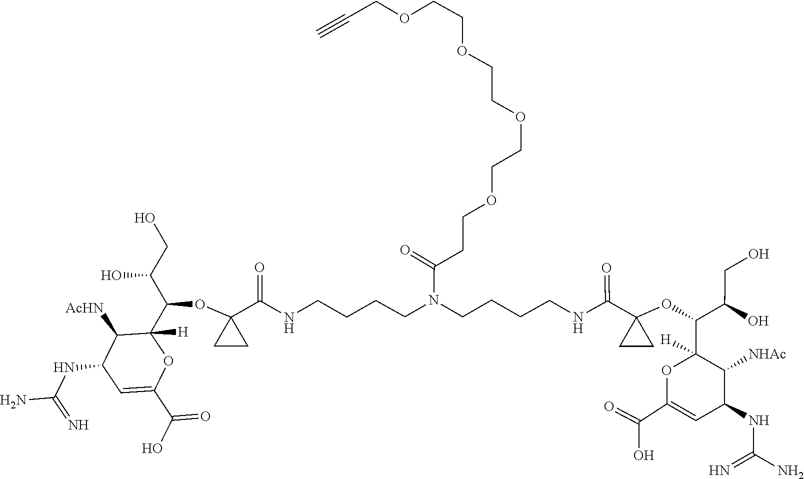 [SMILES: C#CCOCCOCCOCCOCCC(=O)N(CCCCNC(=O)C1(O[C@@H]([C@@H]2OC(C(=O)O)=C[C@H](NC(=N)N)[C@H]2NC(C)=O)[C@H](O)CO)CC1)CCCCNC(=O)C1(O[C@@H]([C@@H]2OC(C(=O)O)=C[C@H](NC(=N)N)[C@H]2NC(C)=O)[C@H](O)CO)CC1] |
| C- Inter- 38 | 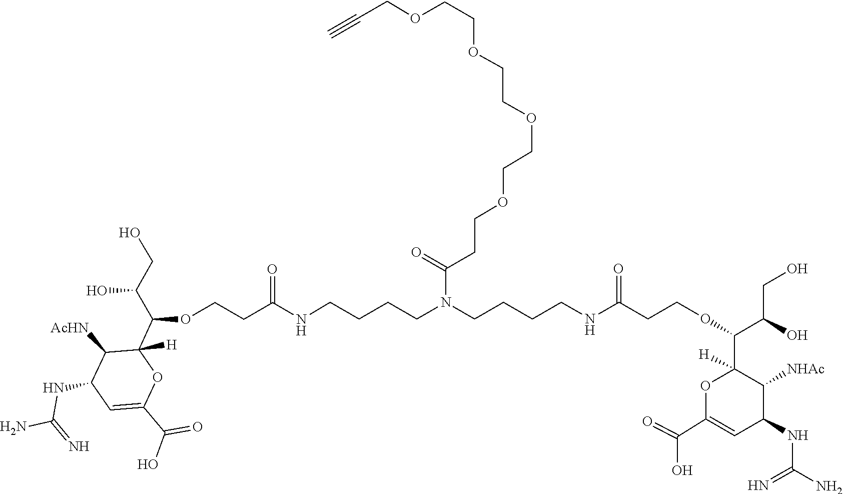 [SMILES: C#CCOCCOCCOCCOCCC(=O)N(CCCCNC(=O)CCO[C@@H]([C@@H]1OC(C(=O)O)=C[C@H](NC(=N)N)[C@H]1NC(C)=O)[C@H](O)CO)CCCCNC(=O)CCO[C@@H]([C@@H]1OC(C(=O)O)=C[C@H](NC(=N)N)[C@H]1NC(C)=O)[C@H](O)CO] |
| C- Inter- 39 | 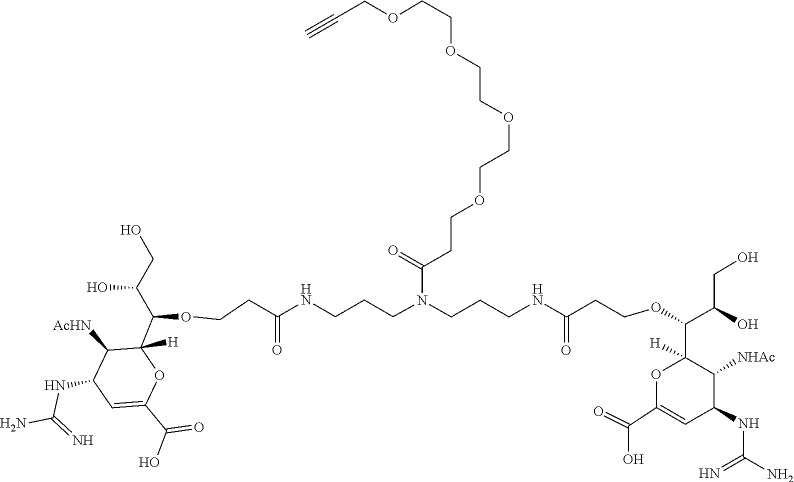 [SMILES: C#CCOCCOCCOCCOCCC(=O)N(CCCNC(=O)CCO[C@@H]([C@@H]1OC(C(=O)O)=C[C@H](NC(=N)N)[C@H]1NC(C)=O)[C@H](O)CO)CCCNC(=O)CCO[C@@H]([C@@H]1OC(C(=O)O)=C[C@H](NC(=N)N)[C@H]1NC(C)=O)[C@H](O)CO] |
| C- Inter- 40 | 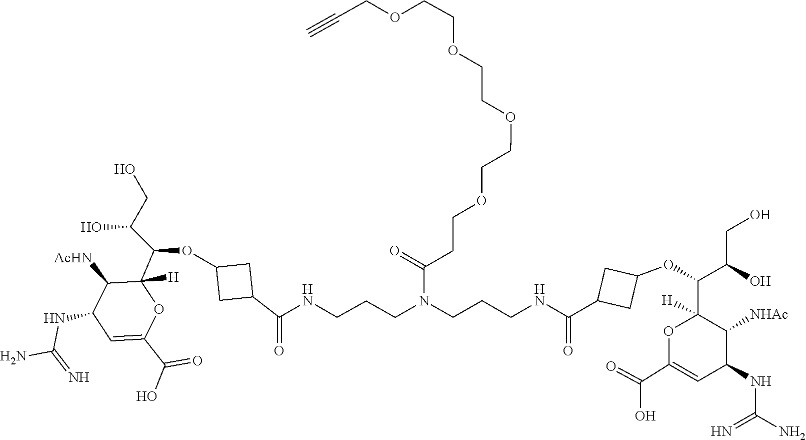 |
| C- Inter- 41 | 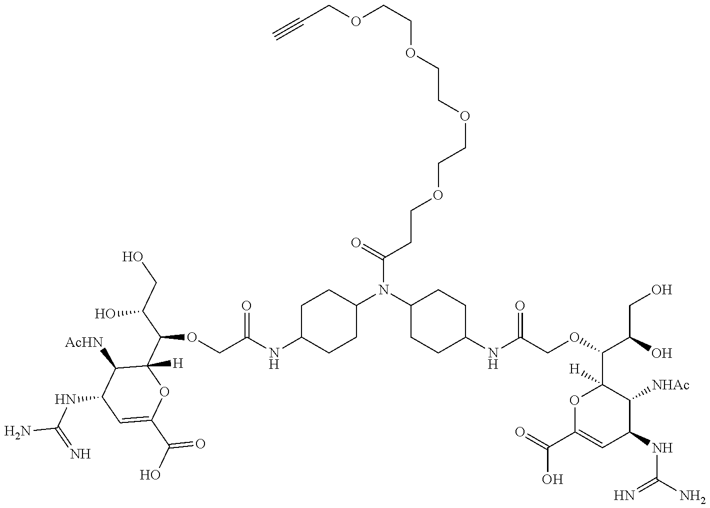 [SMILES: C#CCOCCOCCOCCOCCC(=O)N(C1CCC(NC(=O)CO[C@@H]([C@@H]2OC(C(=O)O)=C[C@H](NC(=N)N)[C@H]2NC(C)=O)[C@H](O)CO)CC1)C1CCC(NC(=O)CO[C@@H]([C@@H]2OC(C(=O)O)=C[C@H](NC(=N)N)[C@H]2NC(C)=O)[C@H](O)CO)CC1] |
| C- Inter- 42 | 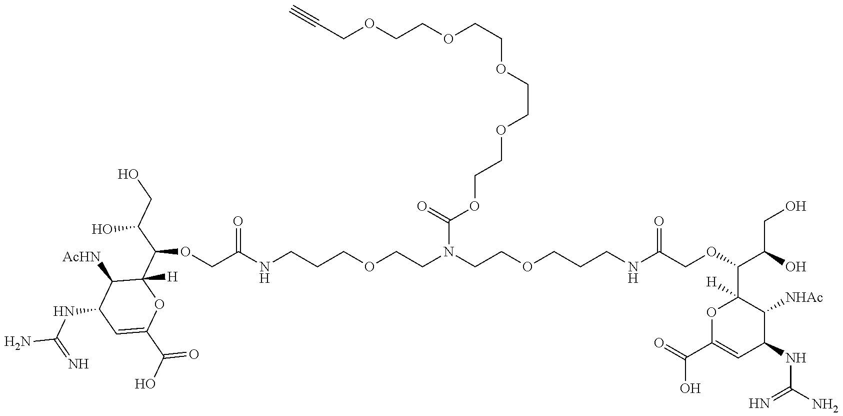 [SMILES: C#CCOCCOCCOCCOCCOC(=O)N(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| C- Inter- 43 | 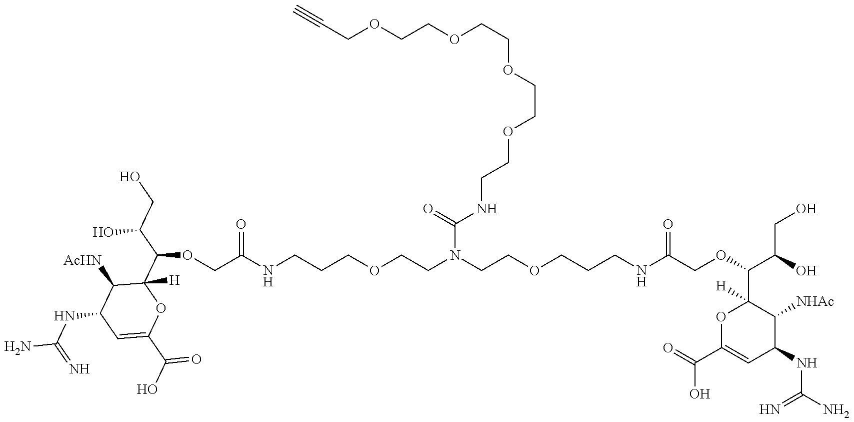 [SMILES: C#CCOCCOCCOCCOCCNC(=O)N(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| C- Inter- 44 | 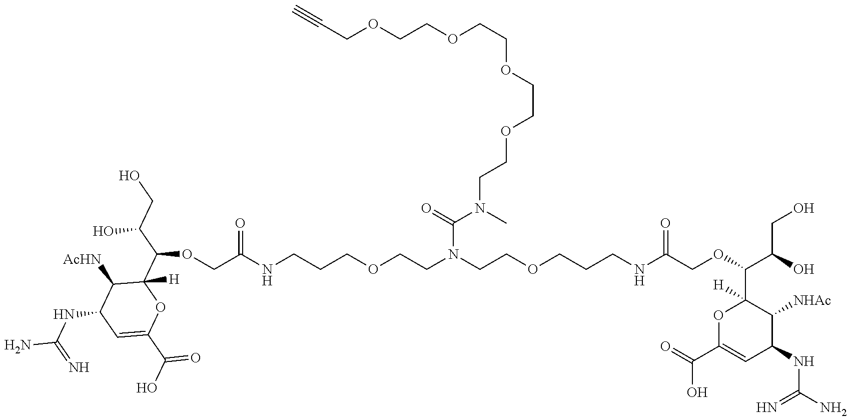 [SMILES: C#CCOCCOCCOCCOCCN(C)C(=O)N(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| C- Inter- 45 | 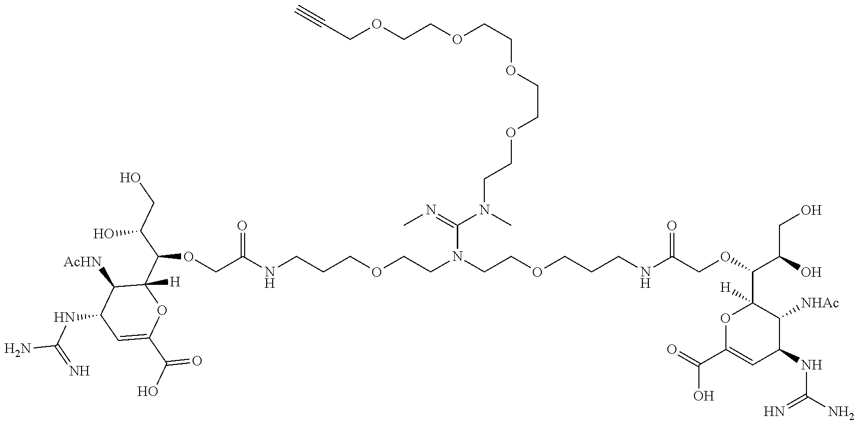 [SMILES: C#CCOCCOCCOCCOCCN(C)/C(=N/C)N(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| C- Inter- 46 | 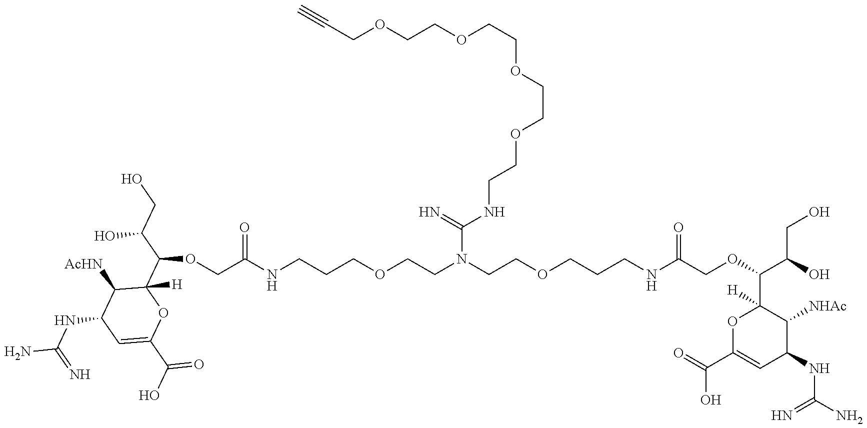 [SMILES: C#CCOCCOCCOCCOCCNC(=N)N(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| C- Inter- 47 | 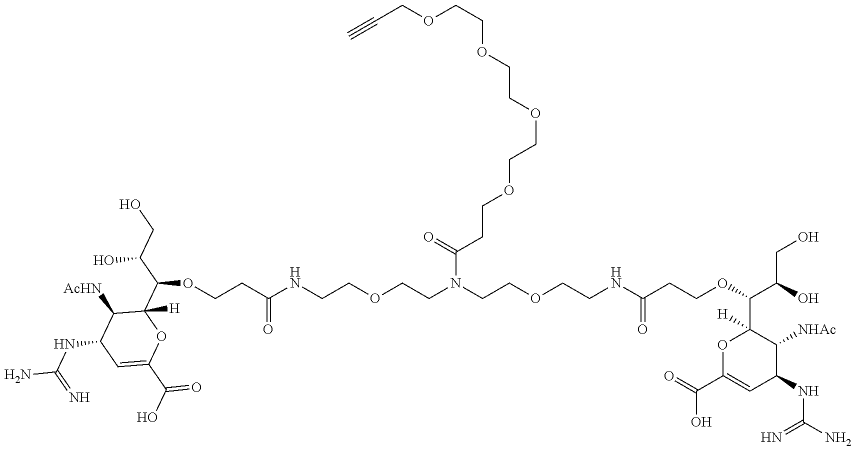 [SMILES: C#CCOCCOCCOCCOCCC(=O)N(CCOCCNC(=O)CCO[C@@H]([C@@H]1OC(C(=O)O)=C[C@H](NC(=N)N)[C@H]1NC(C)=O)[C@H](O)CO)CCOCCNC(=O)CCO[C@@H]([C@@H]1OC(C(=O)O)=C[C@H](NC(=N)N)[C@H]1NC(C)=O)[C@H](O)CO] |
| C- Inter- 48 | 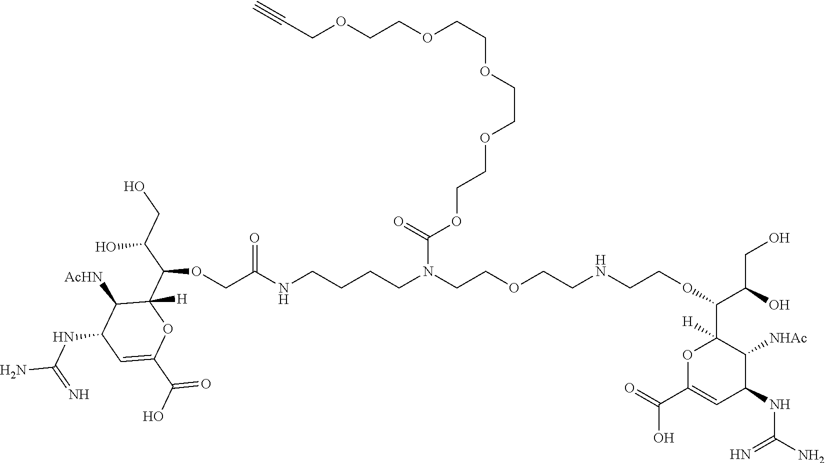 [SMILES: C#CCOCCOCCOCCOCCOC(=O)N(CCCCNC(=O)CO[C@@H]([C@@H]1OC(C(=O)O)=C[C@H](NC(=N)N)[C@H]1NC(C)=O)[C@H](O)CO)CCOCCNCCO[C@@H]([C@@H]1OC(C(=O)O)=C[C@H](NC(=N)N)[C@H]1NC(C)=O)[C@H](O)CO] |
| C- Inter- 49 | 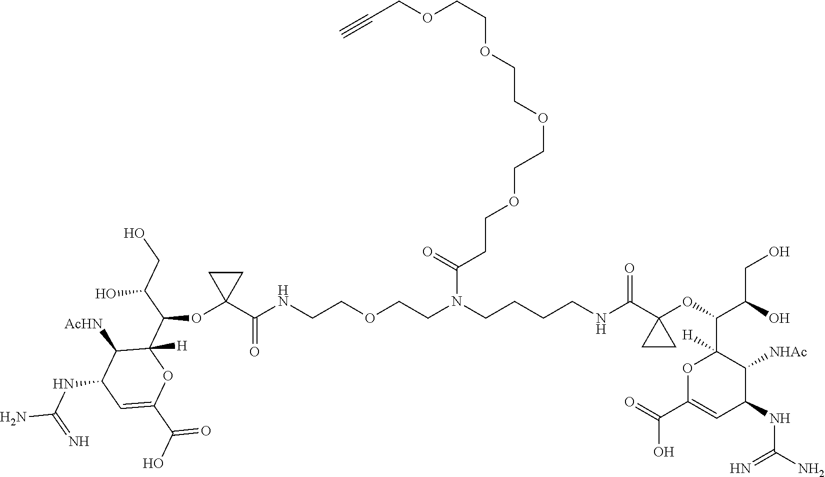 [SMILES: C#CCOCCOCCOCCOCCC(=O)N(CCCCNC(=O)C1(O[C@@H]([C@@H]2OC(C(=O)O)=C[C@H](NC(=N)N)[C@H]2NC(C)=O)[C@H](O)CO)CC1)CCOCCNC(=O)C1(O[C@@H]([C@@H]2OC(C(=O)O)=C[C@H](NC(=N)N)[C@H]2NC(C)=O)[C@H](O)CO)CC1] |
| C- Inter- 50 | 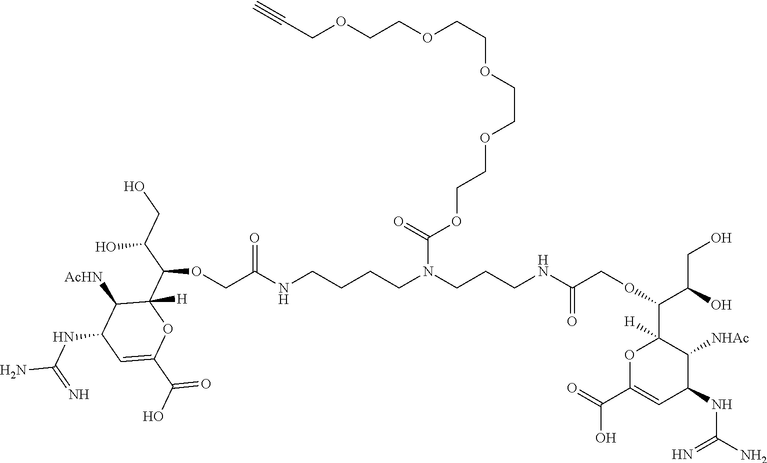 [SMILES: C#CCOCCOCCOCCOCCOC(=O)N(CCCCNC(=O)CO[C@@H]([C@@H]1OC(C(=O)O)=C[C@H](NC(=N)N)[C@H]1NC(C)=O)[C@H](O)CO)CCCNC(=O)CO[C@@H]([C@@H]1OC(C(=O)O)=C[C@H](NC(=N)N)[C@H]1NC(C)=O)[C@H](O)CO] |
| C- Inter- 51 | 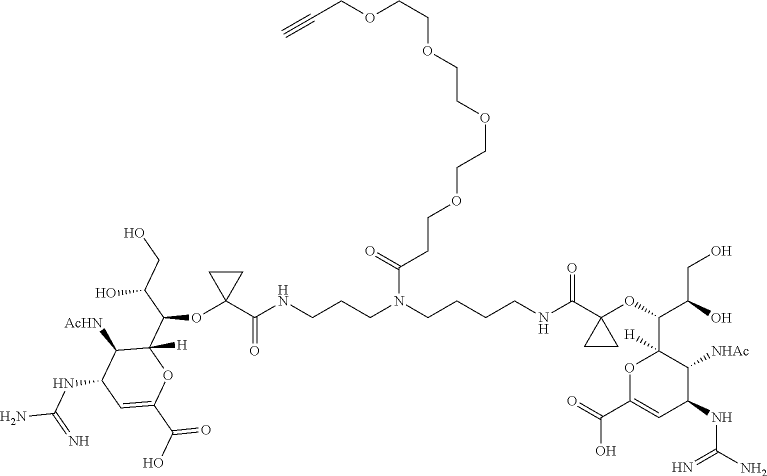 [SMILES: C#CCOCCOCCOCCOCCC(=O)N(CCCCNC(=O)C1(O[C@@H]([C@@H]2OC(C(=O)O)=C[C@H](NC(=N)N)[C@H]2NC(C)=O)[C@H](O)CO)CC1)CCCNC(=O)C1(O[C@@H]([C@@H]2OC(C(=O)O)=C[C@H](NC(=N)N)[C@H]2NC(C)=O)[C@H](O)CO)CC1] |
| C- Inter- 52 | 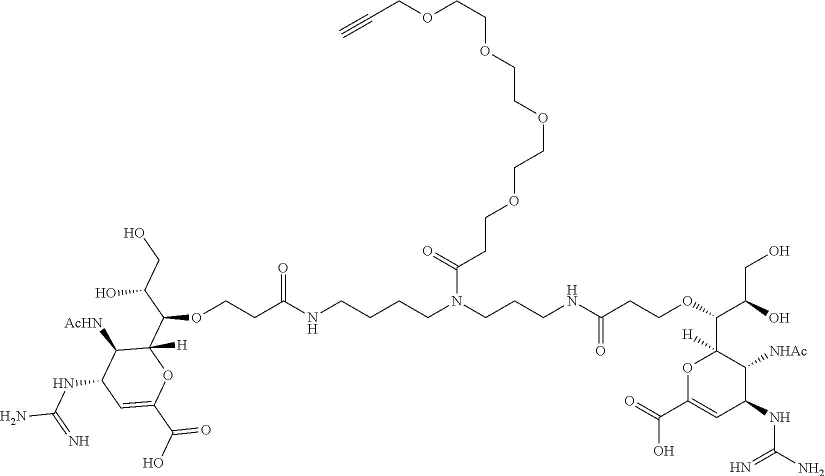 [SMILES: C#CCOCCOCCOCCOCCC(=O)N(CCCCNC(=O)CCO[C@@H]([C@@H]1OC(C(=O)O)=C[C@H](NC(=N)N)[C@H]1NC(C)=O)[C@H](O)CO)CCCNC(=O)CCO[C@@H]([C@@H]1OC(C(=O)O)=C[C@H](NC(=N)N)[C@H]1NC(C)=O)[C@H](O)CO] |
| C- Inter- 53 | 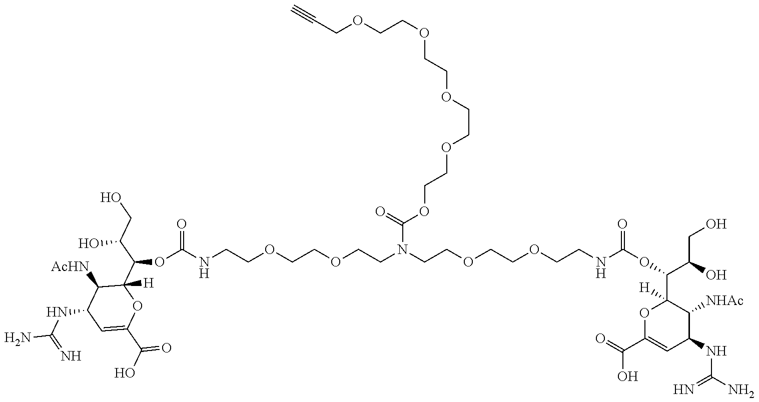 [SMILES: C#CCOCCOCCOCCOCCOC(=O)N(CCOCCOCCNC(=O)O[C@@H]([C@@H]1OC(C(=O)O)=C[C@H](NC(=N)N)[C@H]1NC(C)=O)[C@H](O)CO)CCOCCOCCNC(=O)O[C@@H]([C@@H]1OC(C(=O)O)=C[C@H](NC(=N)N)[C@H]1NC(C)=O)[C@H](O)CO] |
| C- Inter- 54 | 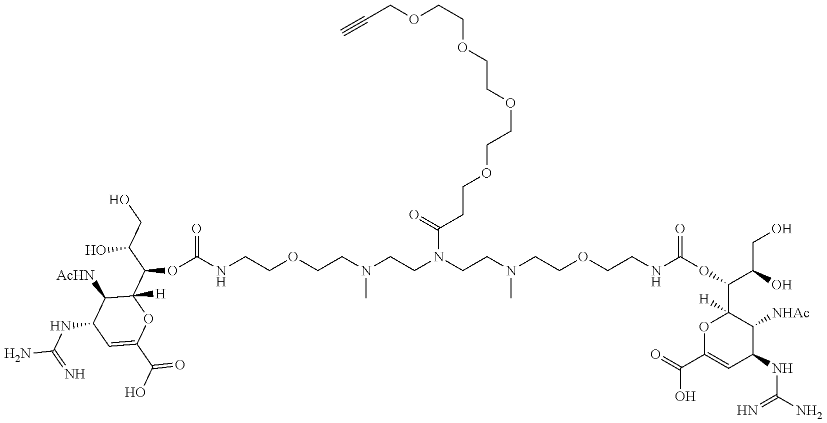 [SMILES: C#CCOCCOCCOCCOCCC(=O)N(CCN(C)CCOCCNC(=O)O[C@@H]([C@@H]1OC(C(=O)O)=C[C@H](NC(=N)N)[C@H]1NC(C)=O)[C@H](O)CO)CCN(C)CCOCCNC(=O)O[C@@H]([C@@H]1OC(C(=O)O)=C[C@H](NC(=N)N)[C@H]1NC(C)=O)[C@H](O)CO] |
| C- Inter- 55 | 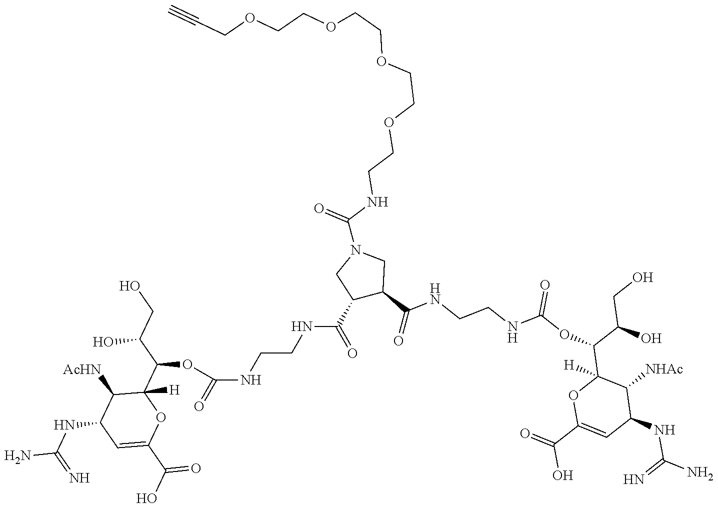 [SMILES: C#CCOCCOCCOCCOCCNC(=O)N1C[C@@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C- Inter- 56 | 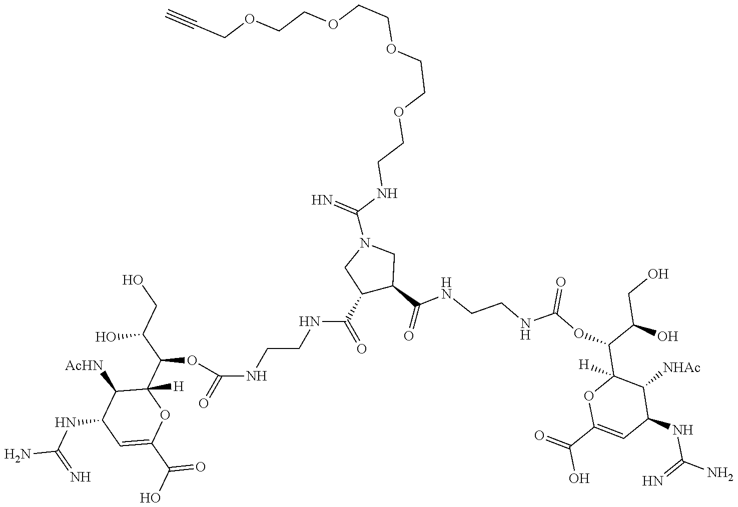 [SMILES: C#CCOCCOCCOCCOCCNC(=N)N1C[C@@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C- Inter- 57 | 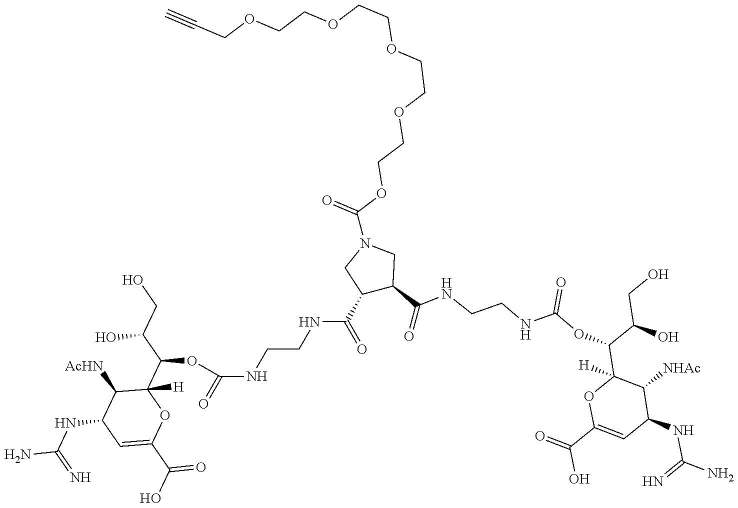 [SMILES: C#CCOCCOCCOCCOCCOC(=O)N1C[C@@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C- Inter- 58 | 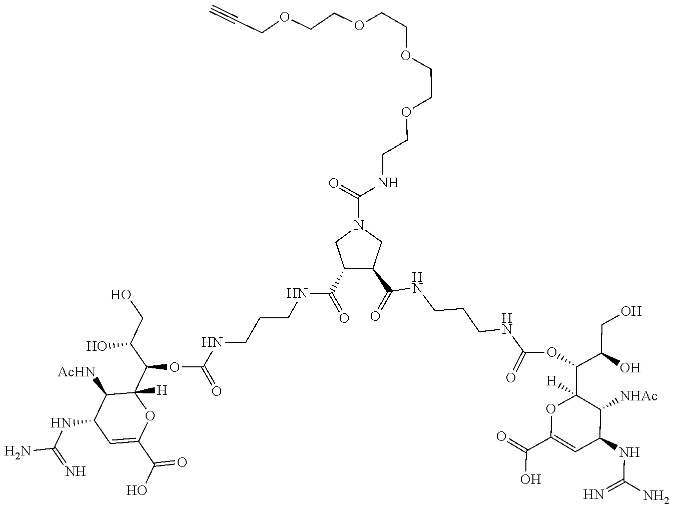 [SMILES: C#CCOCCOCCOCCOCCNC(=O)N1C[C@@H](C(=O)NCCCNC(=O)O[C@@H]([C@@H]2OC(C(=O)O)=C[C@H](NC(=N)N)[C@H]2NC(C)=O)[C@H](O)CO)[C@H](C(=O)NCCCNC(=O)O[C@@H]([C@@H]2OC(C(=O)O)=C[C@H](NC(=N)N)[C@H]2NC(C)=O)[C@H](O)CO)C1] |
| C- Inter- 59 | 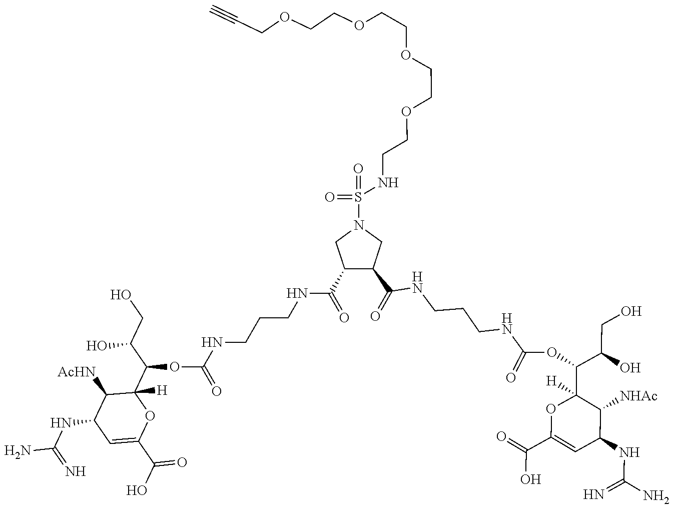 [SMILES: C#CCOCCOCCOCCOCCNS(=O)(=O)N1C[C@@H](C(=O)NCCCNC(=O)O[C@@H]([C@@H]2OC(C(=O)O)=C[C@H](NC(=N)N)[C@H]2NC(C)=O)[C@H](O)CO)[C@H](C(=O)NCCCNC(=O)O[C@@H]([C@@H]2OC(C(=O)O)=C[C@H](NC(=N)N)[C@H]2NC(C)=O)[C@H](O)CO)C1] |
| C- Inter- 60 | 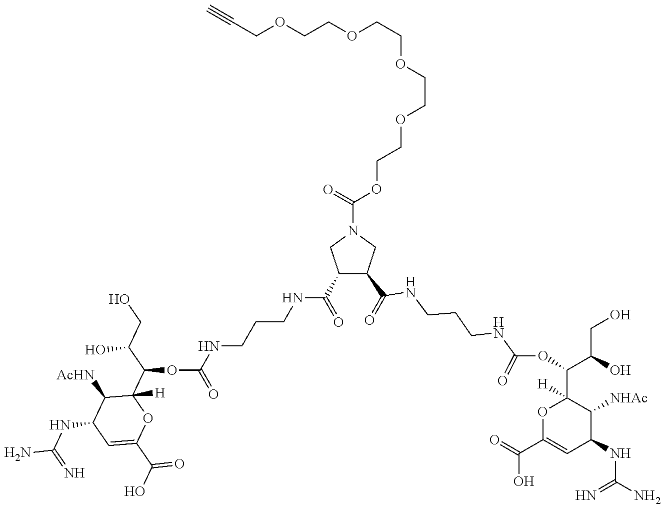 [SMILES: C#CCOCCOCCOCCOCCOC(=O)N1C[C@@H](C(=O)NCCCNC(=O)O[C@@H]([C@@H]2OC(C(=O)O)=C[C@H](NC(=N)N)[C@H]2NC(C)=O)[C@H](O)CO)[C@H](C(=O)NCCCNC(=O)O[C@@H]([C@@H]2OC(C(=O)O)=C[C@H](NC(=N)N)[C@H]2NC(C)=O)[C@H](O)CO)C1] |
| C- Inter- 61 | 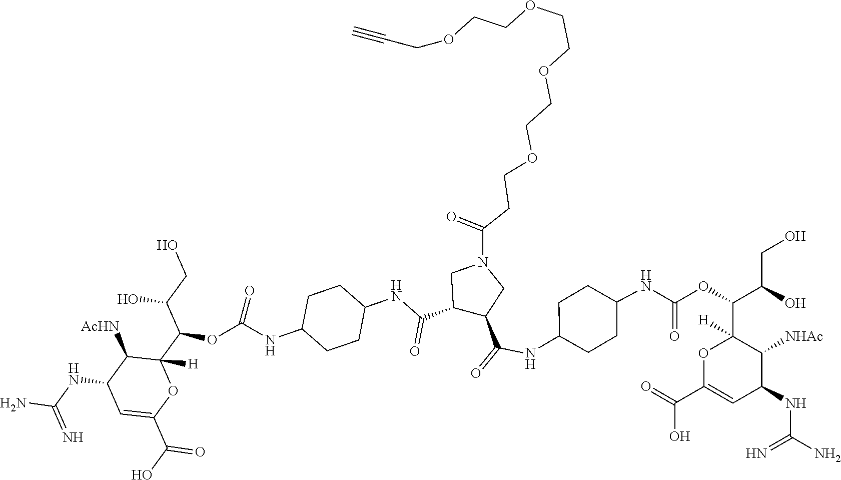 [SMILES: C#CCOCCOCCOCCOCCC(=O)N1C[C@@H](C(=O)NC2CCC(NC(=O)O[C@@H]([C@@H]3OC(C(=O)O)=C[C@H](NC(=N)N)[C@H]3NC(C)=O)[C@H](O)CO)CC2)[C@H](C(=O)NC2CCC(NC(=O)O[C@@H]([C@@H]3OC(C(=O)O)=C[C@H](NC(=N)N)[C@H]3NC(C)=O)[C@H](O)CO)CC2)C1] |
| C- Inter- 62 | 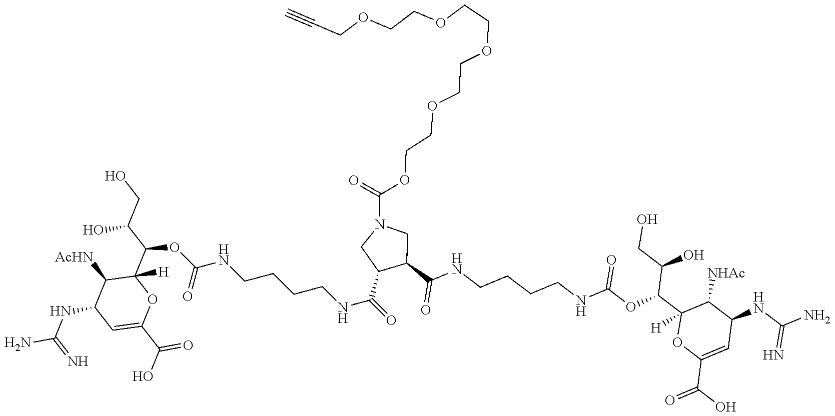 [SMILES: C#CCOCCOCCOCCOCCOC(=O)N1C[C@@H](C(=O)NCCCCNC(=O)O[C@@H]([C@@H]2OC(C(=O)O)=C[C@H](NC(=N)N)[C@H]2NC(C)=O)[C@H](O)CO)[C@H](C(=O)NCCCCNC(=O)O[C@@H]([C@@H]2OC(C(=O)O)=C[C@H](NC(=N)N)[C@H]2NC(C)=O)[C@H](O)CO)C1] |
| C- Inter- 63 | 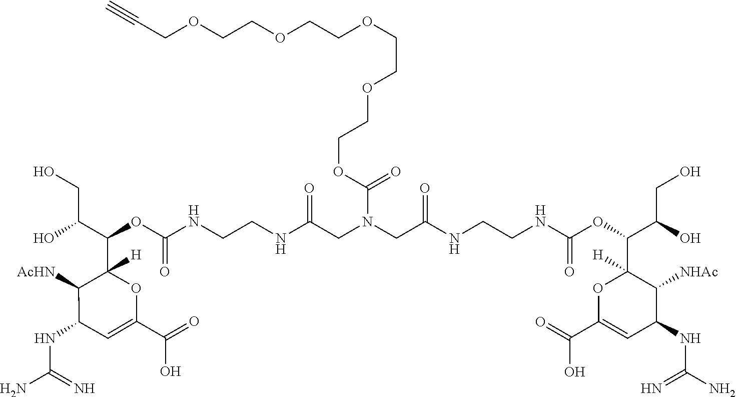 [SMILES: C#CCOCCOCCOCCOCCOC(=O)N(CC(=O)NCCNC(=O)O[C@@H]([C@@H]1OC(C(=O)O)=C[C@H](NC(=N)N)[C@H]1NC(C)=O)[C@H](O)CO)CC(=O)NCCNC(=O)O[C@@H]([C@@H]1OC(C(=O)O)=C[C@H](NC(=N)N)[C@H]1NC(C)=O)[C@H](O)CO] |
| C- Inter- 64 | 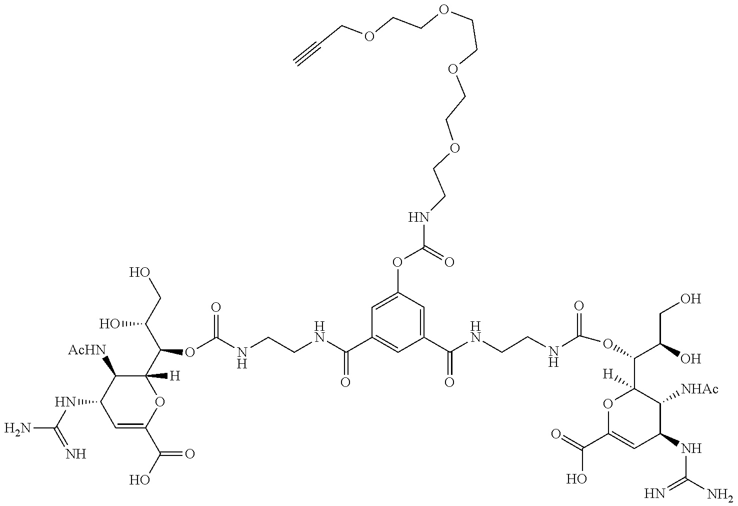 [SMILES: C#CCOCCOCCOCCOCCNC(=O)Oc1cc(C(=O)NCCNC(=O)O[C@@H]([C@@H]2OC(C(=O)O)=C[C@H](NC(=N)N)[C@H]2NC(C)=O)[C@H](O)CO)cc(C(=O)NCCNC(=O)O[C@@H]([C@@H]2OC(C(=O)O)=C[C@H](NC(=N)N)[C@H]2NC(C)=O)[C@H](O)CO)c1] |
| C- Inter- 65 | 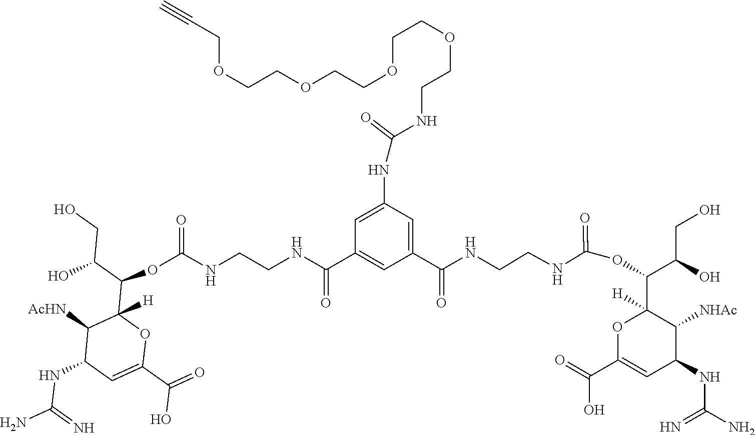 [SMILES: C#CCOCCOCCOCCOCCNC(=O)Nc1cc(C(=O)NCCNC(=O)O[C@@H]([C@@H]2OC(C(=O)O)=C[C@H](NC(=N)N)[C@H]2NC(C)=O)[C@H](O)CO)cc(C(=O)NCCNC(=O)O[C@@H]([C@@H]2OC(C(=O)O)=C[C@H](NC(=N)N)[C@H]2NC(C)=O)[C@H](O)CO)c1] |
| C- Inter- 66 | 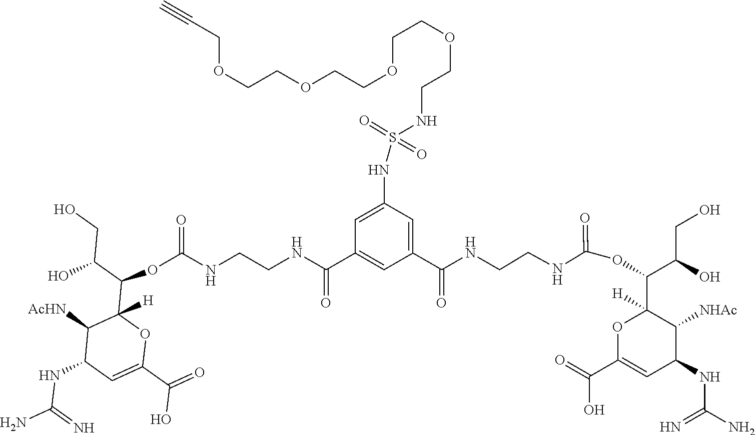 [SMILES: C#CCOCCOCCOCCOCCNS(=O)(=O)Nc1cc(C(=O)NCCNC(=O)O[C@@H]([C@@H]2OC(C(=O)O)=C[C@H](NC(=N)N)[C@H]2NC(C)=O)[C@H](O)CO)cc(C(=O)NCCNC(=O)O[C@@H]([C@@H]2OC(C(=O)O)=C[C@H](NC(=N)N)[C@H]2NC(C)=O)[C@H](O)CO)c1] |
| C- Inter- 67 | 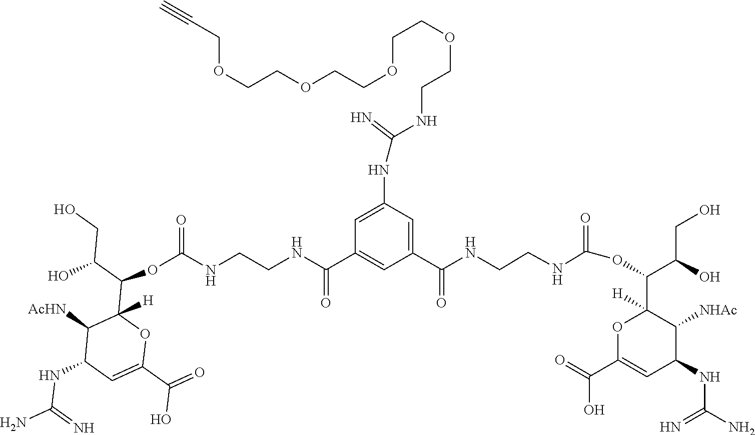 [SMILES: C#CCOCCOCCOCCOCCNC(=N)Nc1cc(C(=O)NCCNC(=O)O[C@@H]([C@@H]2OC(C(=O)O)=C[C@H](NC(=N)N)[C@H]2NC(C)=O)[C@H](O)CO)cc(C(=O)NCCNC(=O)O[C@@H]([C@@H]2OC(C(=O)O)=C[C@H](NC(=N)N)[C@H]2NC(C)=O)[C@H](O)CO)c1] |
| C- Inter- 68 | 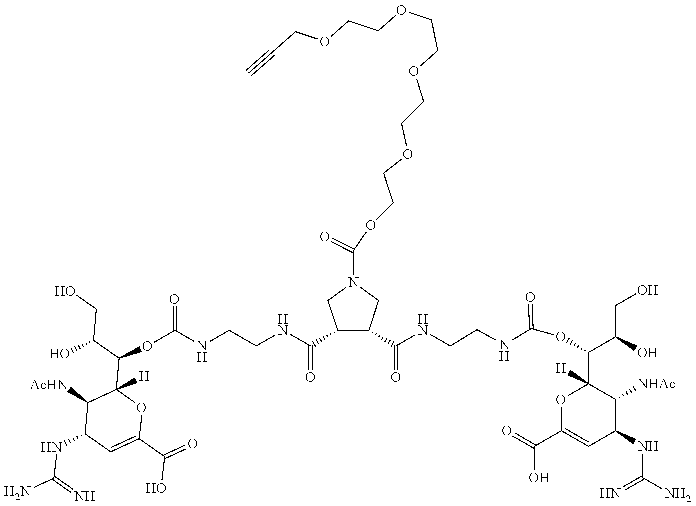 [SMILES: C#CCOCCOCCOCCOCCOC(=O)N1C[C@H](C(=O)NCCNC(=O)O[C@@H]([C@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C- Inter- 69 | 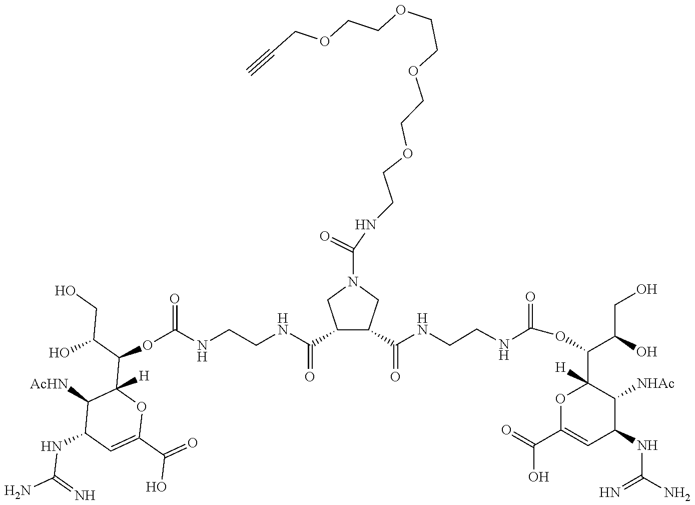 [SMILES: C#CCOCCOCCOCCOCCNC(=O)N1C[C@H](C(=O)NCCNC(=O)O[C@@H]([C@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C- Inter- 70 | 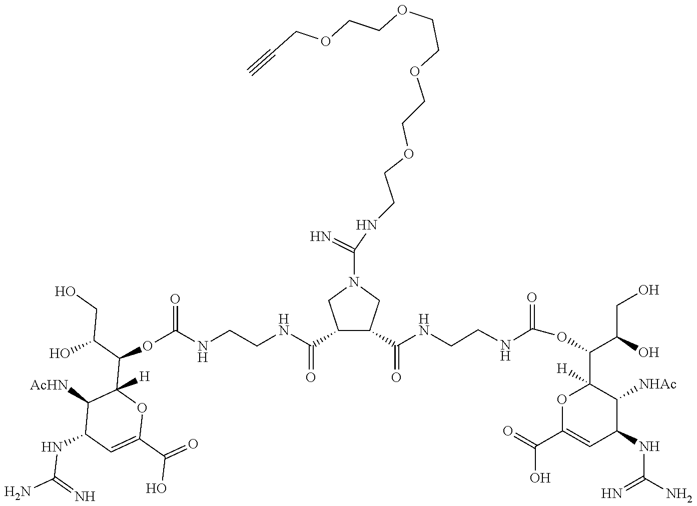 [SMILES: C#CCOCCOCCOCCOCCNC(=N)N1C[C@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C- Inter- 71 | 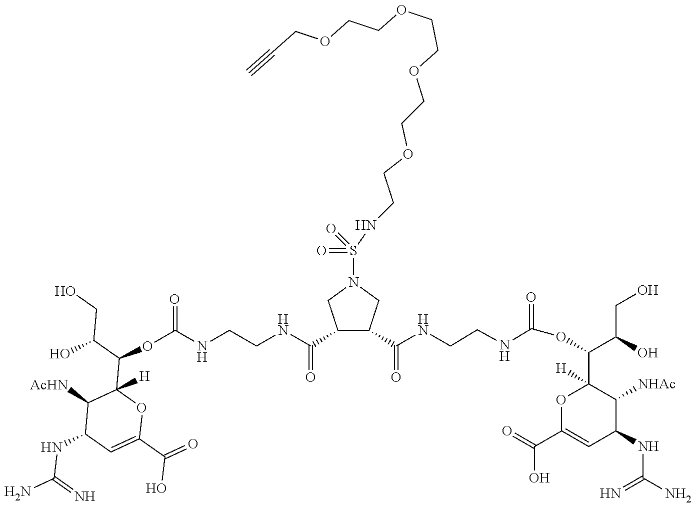 [SMILES: C#CCOCCOCCOCCOCCNS(=O)(=O)N1C[C@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C- Inter- 72 | 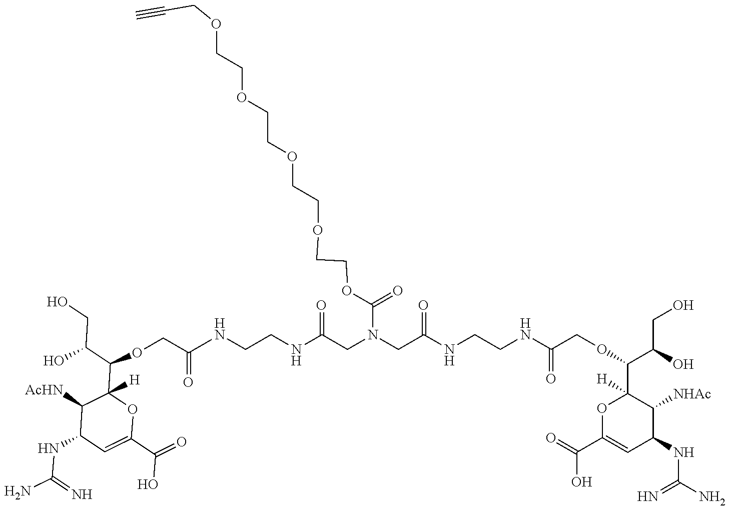 [SMILES: C#CCOCCOCCOCCOCCOC(=O)N(CC(=O)NCCNC(=O)CO[C@@H]([C@@H]1OC(C(=O)O)=C[C@H](NC(=N)N)[C@H]1NC(C)=O)[C@H](O)CO)CC(=O)NCCNC(=O)CO[C@@H]([C@@H]1OC(C(=O)O)=C[C@H](NC(=N)N)[C@H]1NC(C)=O)[C@H](O)CO] |
| C- Inter- 73 | 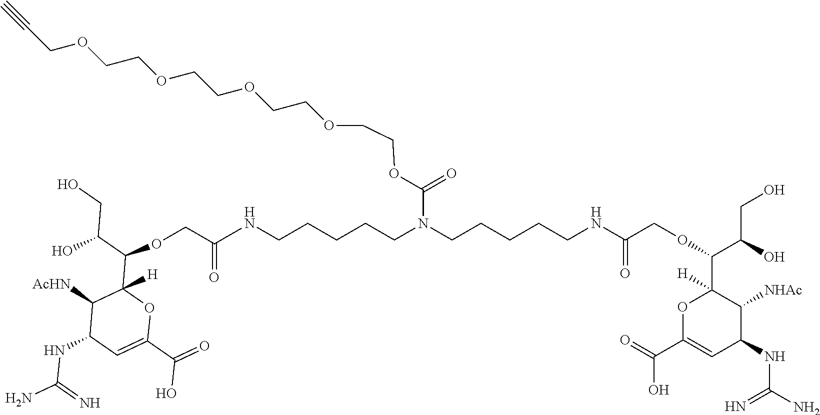 [SMILES: C#CCOCCOCCOCCOCCOC(=O)N(CCCCCNC(=O)CO[C@@H]([C@@H]1OC(C(=O)O)=C[C@H](NC(=N)N)[C@H]1NC(C)=O)[C@H](O)CO)CCCCCNC(=O)CO[C@@H]([C@@H]1OC(C(=O)O)=C[C@H](NC(=N)N)[C@H]1NC(C)=O)[C@H](O)CO] |
| C- Inter- 74 | 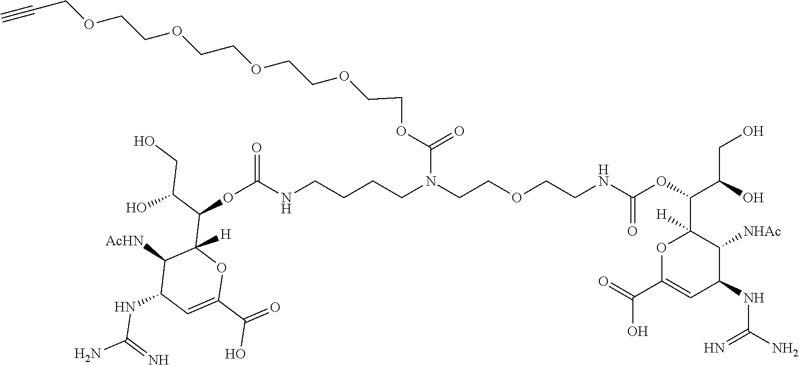 [SMILES: C#CCOCCOCCOCCOCCOC(=O)N(CCCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| C- Inter- 75 | 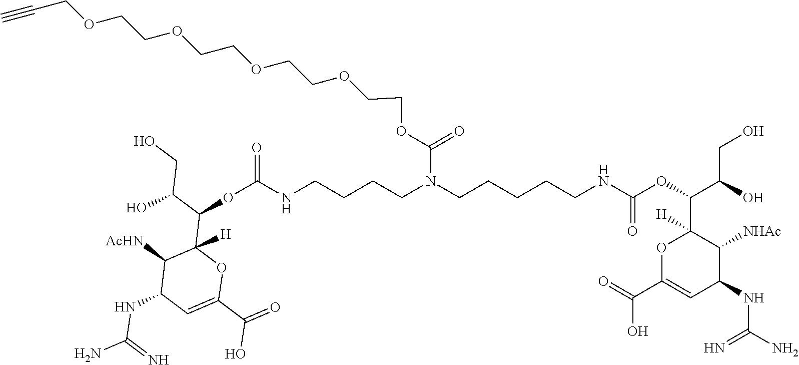 [SMILES: C#CCOCCOCCOCCOCCOC(=O)N(CCCCCNC(=O)O[C@@H]([C@@H]1OC(C(=O)O)=C[C@H](NC(=N)N)[C@H]1NC(C)=O)[C@H](O)CO)CCCCNC(=O)O[C@@H]([C@@H]1OC(C(=O)O)=C[C@H](NC(=N)N)[C@H]1NC(C)=O)[C@H](O)CO] |
| C- Inter- 76 | 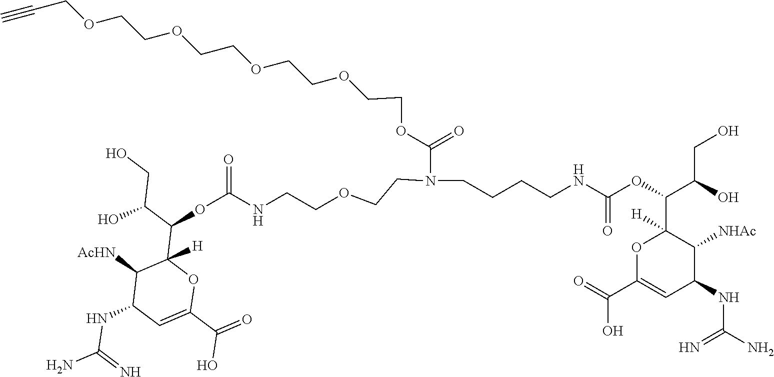 [SMILES: C#CCOCCOCCOCCOCCOC(=O)N(CCCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| C- Inter- 77 | 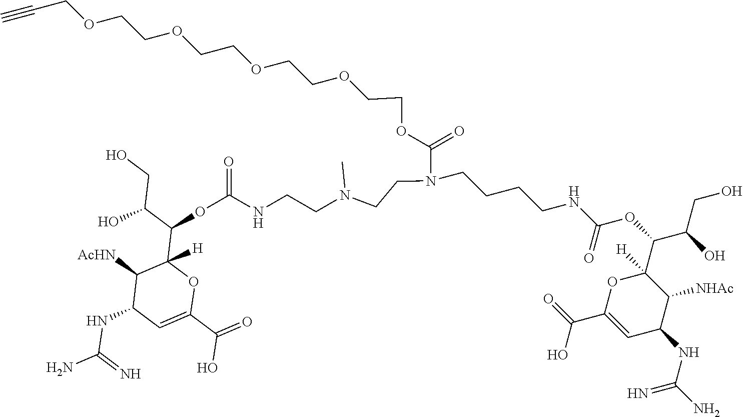 [SMILES: C#CCOCCOCCOCCOCCOC(=O)N(CCCCNC(=O)O[C@@H]([C@@H]1OC(C(=O)O)=C[C@H](NC(=N)N)[C@H]1NC(C)=O)[C@H](O)CO)CCN(C)CCNC(=O)O[C@@H]([C@@H]1OC(C(=O)O)=C[C@H](NC(=N)N)[C@H]1NC(C)=O)[C@H](O)CO] |
| C- Inter- 78 | 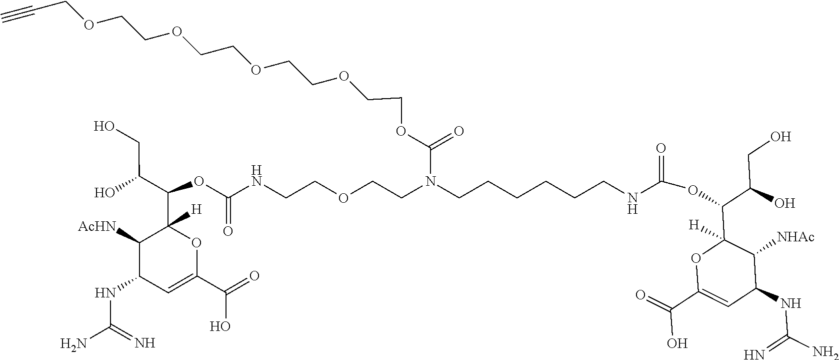 [SMILES: C#CCOCCOCCOCCOCCOC(=O)N(CCCCCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| C- Inter- 79 | 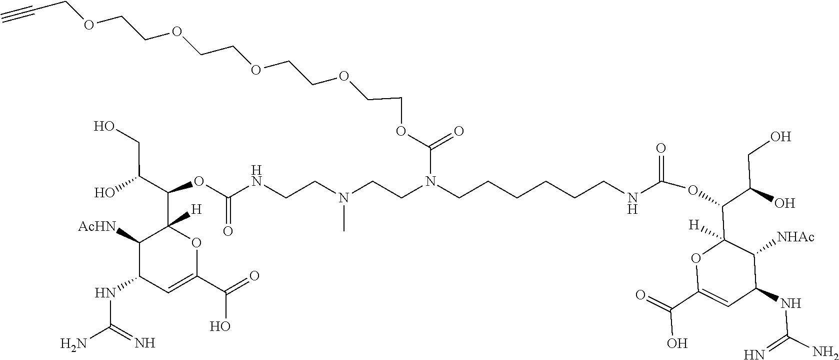 [SMILES: C#CCOCCOCCOCCOCCOC(=O)N(CCCCCCNC(=O)O[C@@H]([C@@H]1OC(C(=O)O)=C[C@H](NC(=N)N)[C@H]1NC(C)=O)[C@H](O)CO)CCN(C)CCNC(=O)O[C@@H]([C@@H]1OC(C(=O)O)=C[C@H](NC(=N)N)[C@H]1NC(C)=O)[C@H](O)CO] |
| C- Inter- 80 | 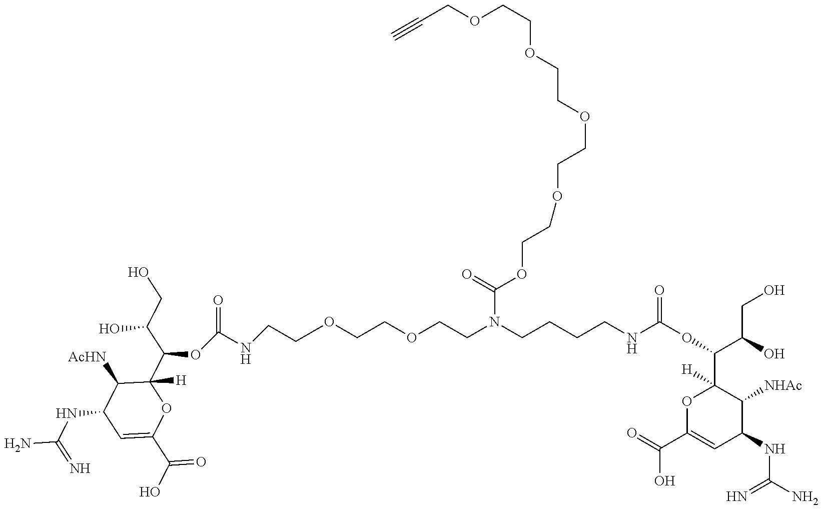 [SMILES: C#CCOCCOCCOCCOCCOC(=O)N(CCCCNC(=O)O[C@@H]([C@@H]1OC(C(=O)O)=C[C@H](NC(=N)N)[C@H]1NC(C)=O)[C@H](O)CO)CCOCCOCCNC(=O)O[C@@H]([C@@H]1OC(C(=O)O)=C[C@H](NC(=N)N)[C@H]1NC(C)=O)[C@H](O)CO] |
| C- Inter- 81 | 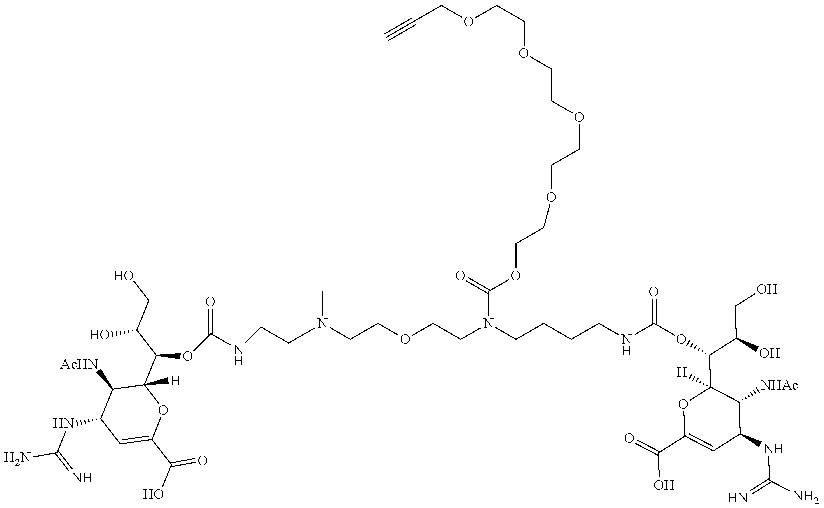 [SMILES: C#CCOCCOCCOCCOCCOC(=O)N(CCCCNC(=O)O[C@@H]([C@@H]1OC(C(=O)O)=C[C@H](NC(=N)N)[C@H]1NC(C)=O)[C@H](O)CO)CCOCCN(C)CCNC(=O)O[C@@H]([C@@H]1OC(C(=O)O)=C[C@H](NC(=N)N)[C@H]1NC(C)=O)[C@H](O)CO] |
| C- Inter- 82 | 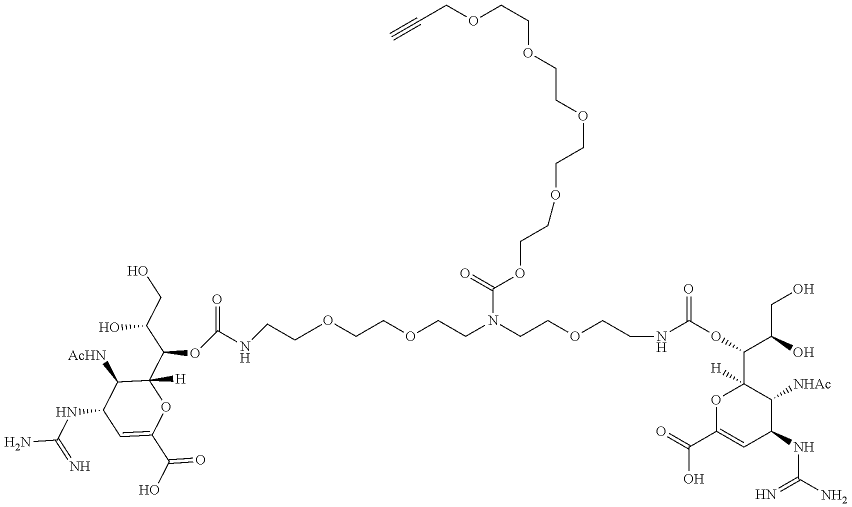 [SMILES: C#CCOCCOCCOCCOCCOC(=O)N(CCOCCNC(=O)O[C@@H]([C@@H]1OC(C(=O)O)=C[C@H](NC(=N)N)[C@H]1NC(C)=O)[C@H](O)CO)CCOCCOCCNC(=O)O[C@@H]([C@@H]1OC(C(=O)O)=C[C@H](NC(=N)N)[C@H]1NC(C)=O)[C@H](O)CO] |
| C- Inter- 83 | 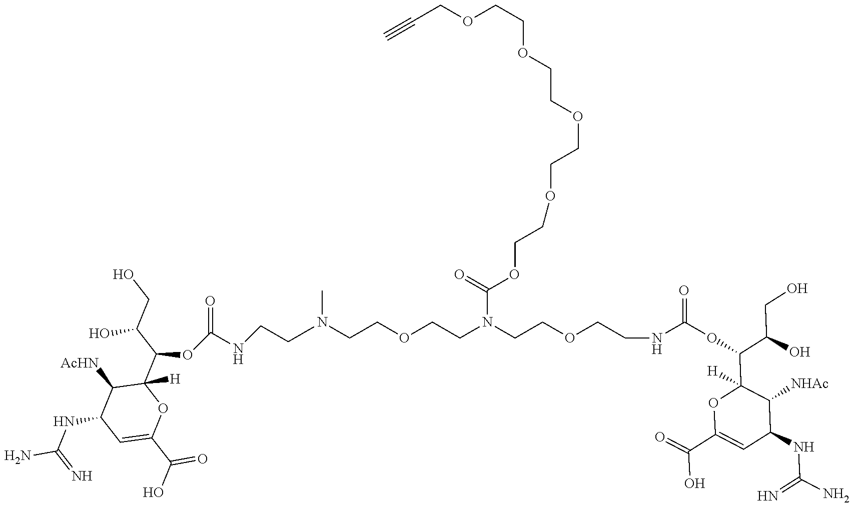 [SMILES: C#CCOCCOCCOCCOCCOC(=O)N(CCOCCNC(=O)O[C@@H]([C@@H]1OC(C(=O)O)=C[C@H](NC(=N)N)[C@H]1NC(C)=O)[C@H](O)CO)CCOCCN(C)CCNC(=O)O[C@@H]([C@@H]1OC(C(=O)O)=C[C@H](NC(=N)N)[C@H]1NC(C)=O)[C@H](O)CO] |
| C- Inter- 84 | 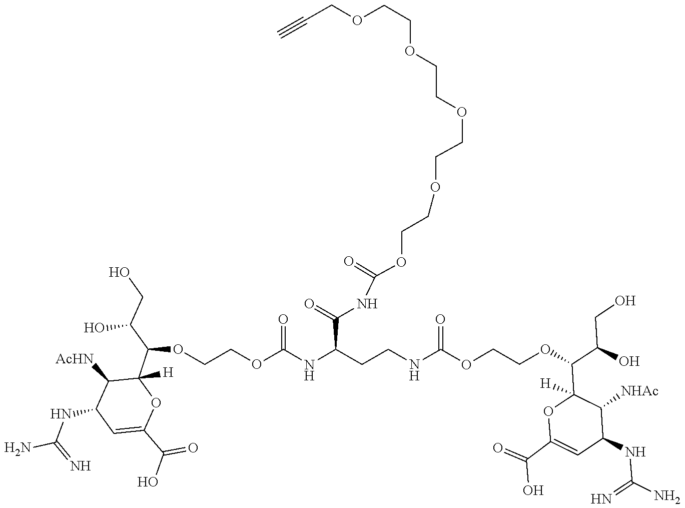 [SMILES: C#CCOCCOCCOCCOCCOC(=O)NC(=O)[C@@H](CCNC(=O)OCCO[C@@H]([C@@H]1OC(C(=O)O)=C[C@H](NC(=N)N)[C@H]1NC(C)=O)[C@H](O)CO)NC(=O)OCCO[C@@H]([C@@H]1OC(C(=O)O)=C[C@H](NC(=N)N)[C@H]1NC(C)=O)[C@H](O)CO] |
| C- Inter- 85 | 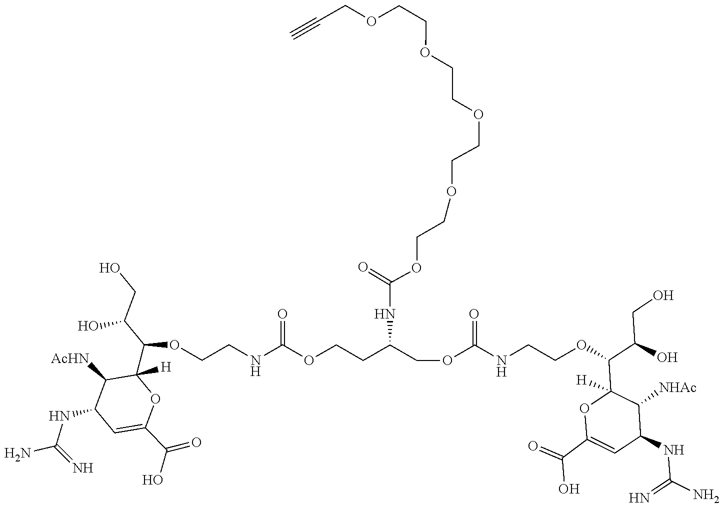 [SMILES: C#CCOCCOCCOCCOCCOC(=O)N[C@@H](CCOC(=O)NCCO[C@@H]([C@@H]1OC(C(=O)O)=C[C@H](NC(=N)N)[C@H]1NC(C)=O)[C@H](O)CO)COC(=O)NCCO[C@@H]([C@@H]1OC(C(=O)O)=C[C@H](NC(=N)N)[C@H]1NC(C)=O)[C@H](O)CO] |
| C- Inter- 86 | 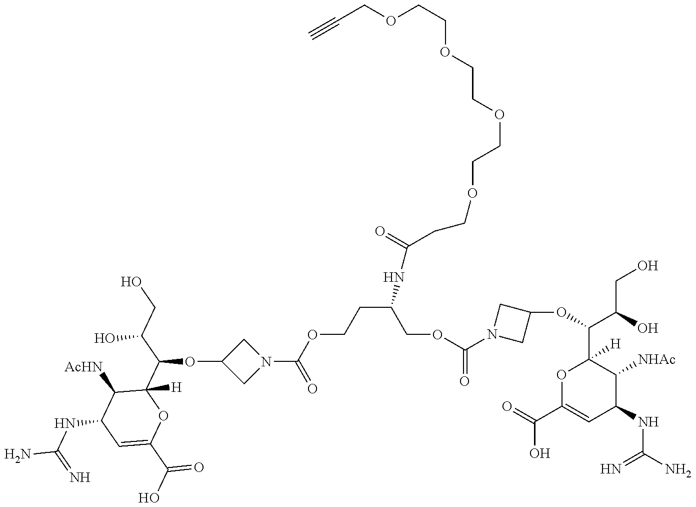 [SMILES: C#CCOCCOCCOCCOCCC(=O)N[C@@H](CCOC(=O)N1CC(O[C@@H]([C@@H]2OC(C(=O)O)=C[C@H](NC(=N)N)[C@H]2NC(C)=O)[C@H](O)CO)C1)COC(=O)N1CC(O[C@@H]([C@@H]2OC(C(=O)O)=C[C@H](NC(=N)N)[C@H]2NC(C)=O)[C@H](O)CO)C1] |
| C- Inter- 87 | 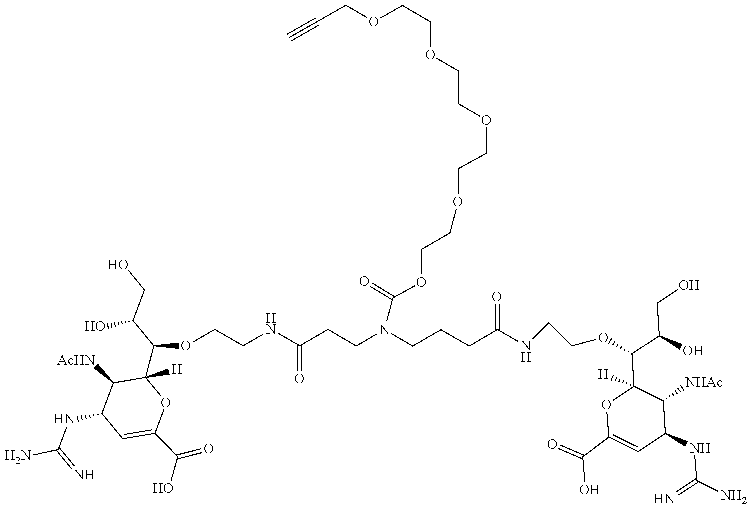 [SMILES: C#CCOCCOCCOCCOCCOC(=O)N(CCCC(=O)NCCO[C@@H]([C@@H]1OC(C(=O)O)=C[C@H](NC(=N)N)[C@H]1NC(C)=O)[C@H](O)CO)CCC(=O)NCCO[C@@H]([C@@H]1OC(C(=O)O)=C[C@H](NC(=N)N)[C@H]1NC(C)=O)[C@H](O)CO] |
| C- Inter- 88 | 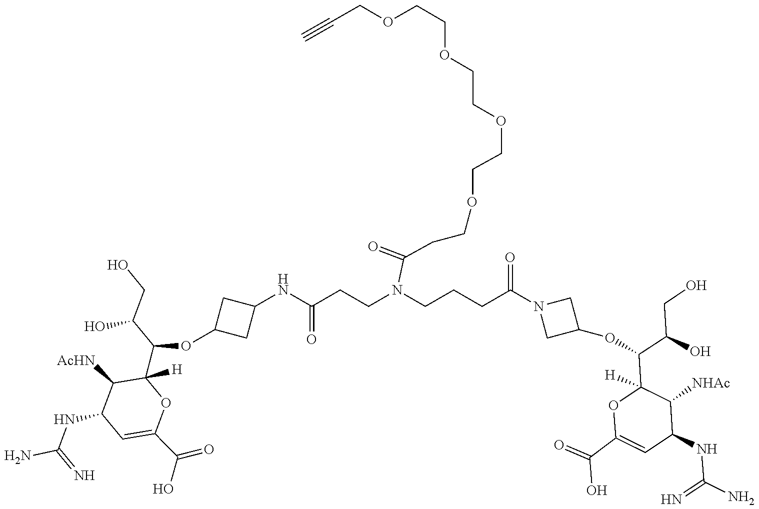 [SMILES: C#CCOCCOCCOCCOCCC(=O)N(CCCC(=O)N1CC(O[C@@H]([C@@H]2OC(C(=O)O)=C[C@H](NC(=N)N)[C@H]2NC(C)=O)[C@H](O)CO)C1)CCC(=O)NC1CC(O[C@@H]([C@@H]2OC(C(=O)O)=C[C@H](NC(=N)N)[C@H]2NC(C)=O)[C@H](O)CO)C1] |
| C- Inter- 89 | 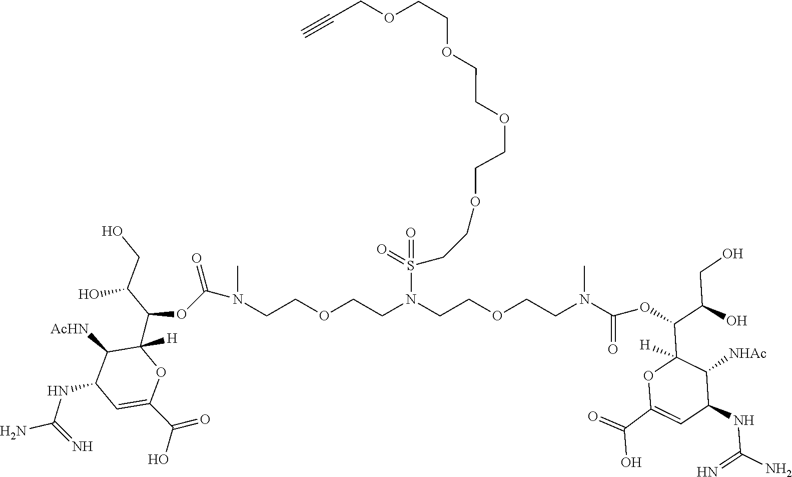 [SMILES: C#CCOCCOCCOCCOCCS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C- Inter- 90 | 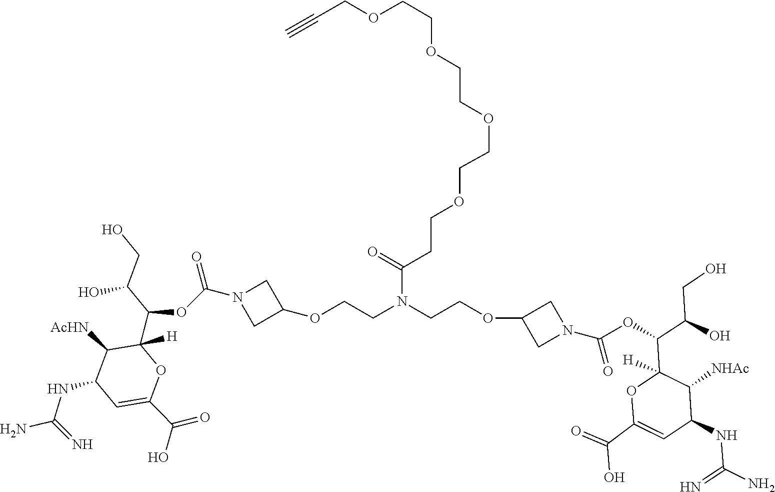 [SMILES: C#CCOCCOCCOCCOCCC(=O)N(CCOC1CN(C(=O)O[C@@H]([C@@H]2OC(C(=O)O)=C[C@H](NC(=N)N)[C@H]2NC(C)=O)[C@H](O)CO)C1)CCOC1CN(C(=O)O[C@@H]([C@@H]2OC(C(=O)O)=C[C@H](NC(=N)N)[C@H]2NC(C)=O)[C@H](O)CO)C1] |
| C- Inter- 91 | 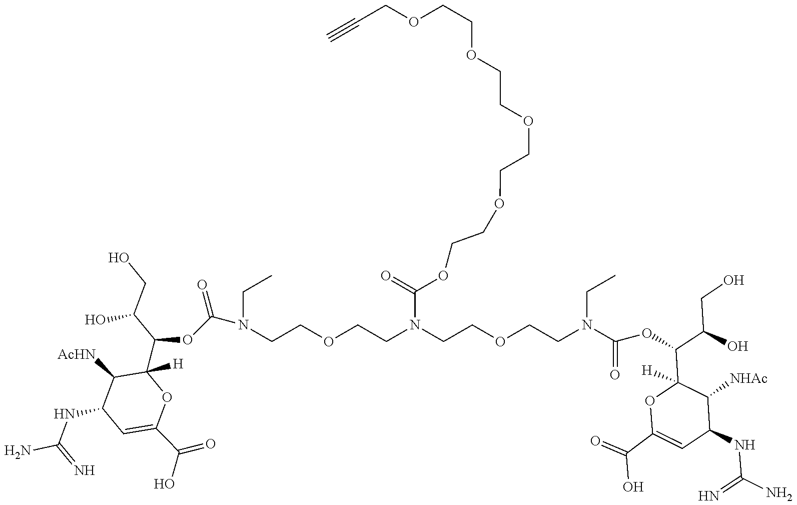 [SMILES: C#CCOCCOCCOCCOCCOC(=O)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C- Inter- 92 | 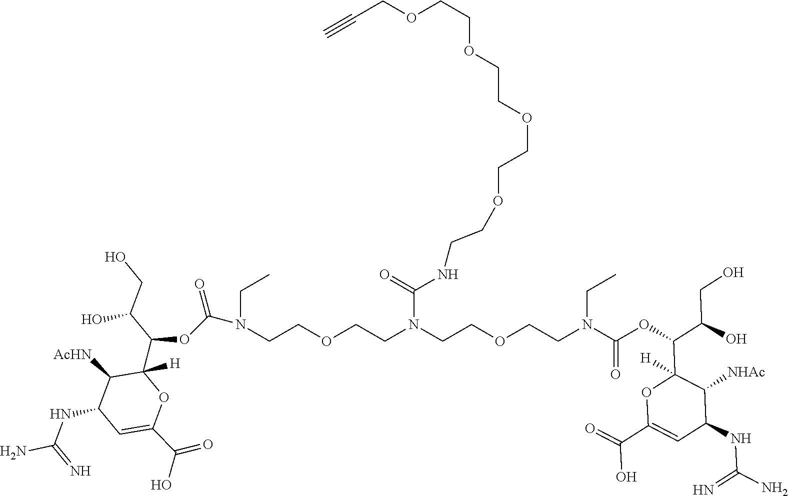 [SMILES: C#CCOCCOCCOCCOCCNC(=O)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C- Inter- 93 | 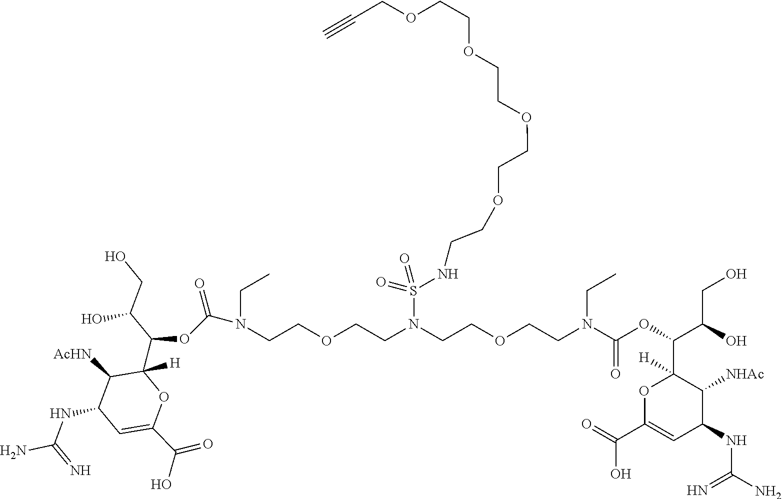 [SMILES: C#CCOCCOCCOCCOCCNS(=O)(=O)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C- Inter- 94 | 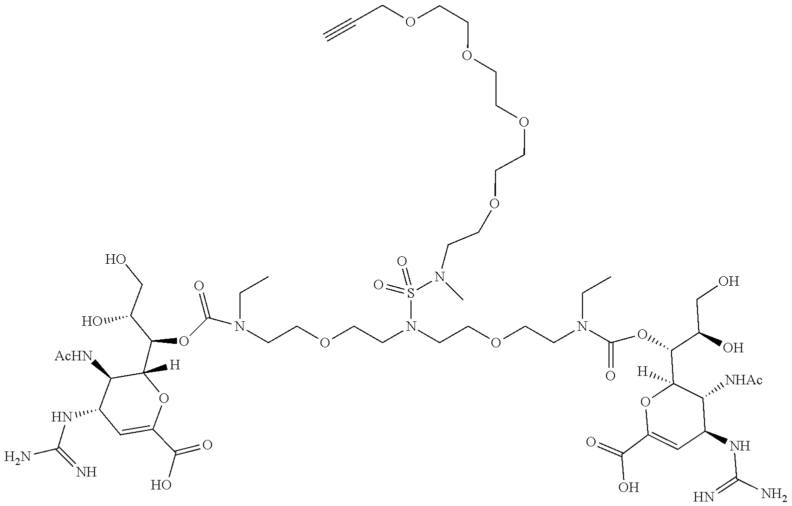 [SMILES: C#CCOCCOCCOCCOCCN(C)S(=O)(=O)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C- Inter- 95 | 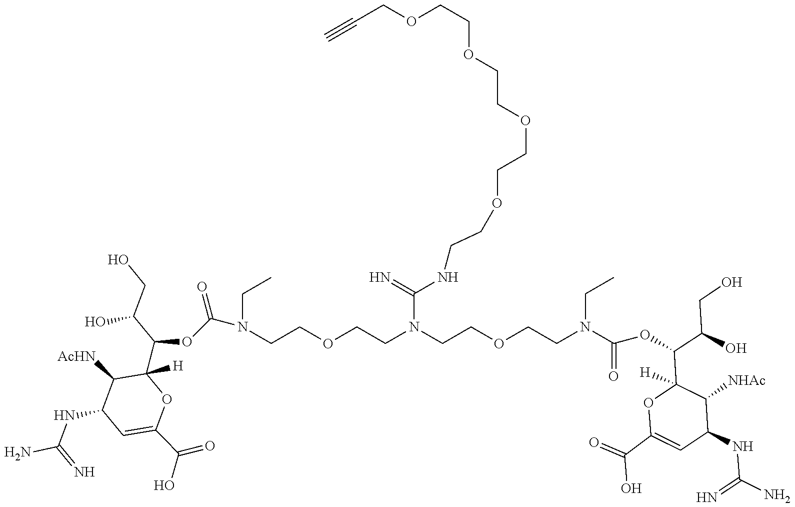 [SMILES: C#CCOCCOCCOCCOCCNC(=N)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C- Inter- 96 | 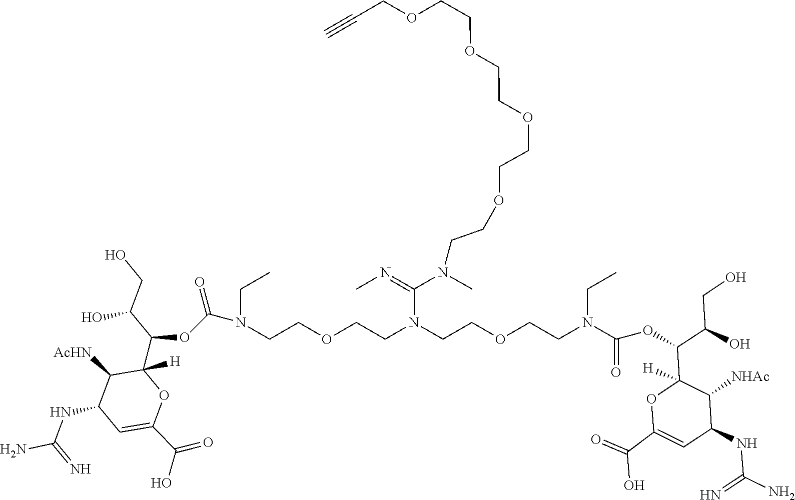 [SMILES: C#CCOCCOCCOCCOCCN(C)/C(=N/C)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C- Inter- 97 | 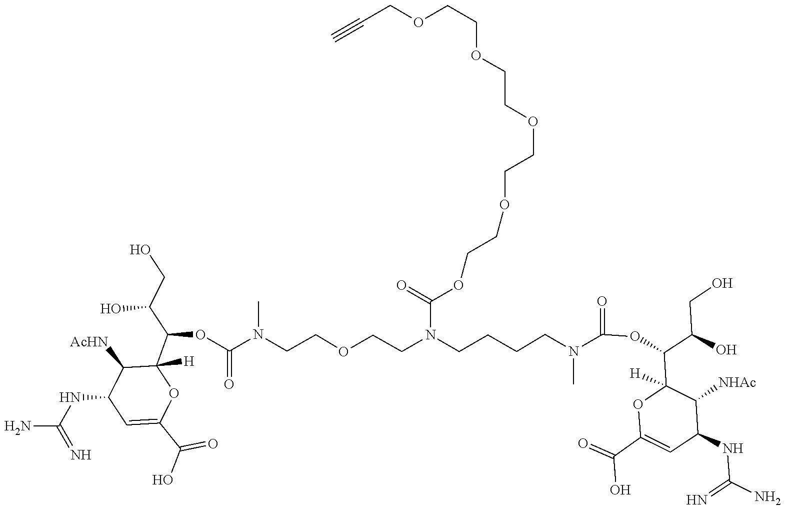 [SMILES: C#CCOCCOCCOCCOCCOC(=O)N(CCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C- Inter- 98 | 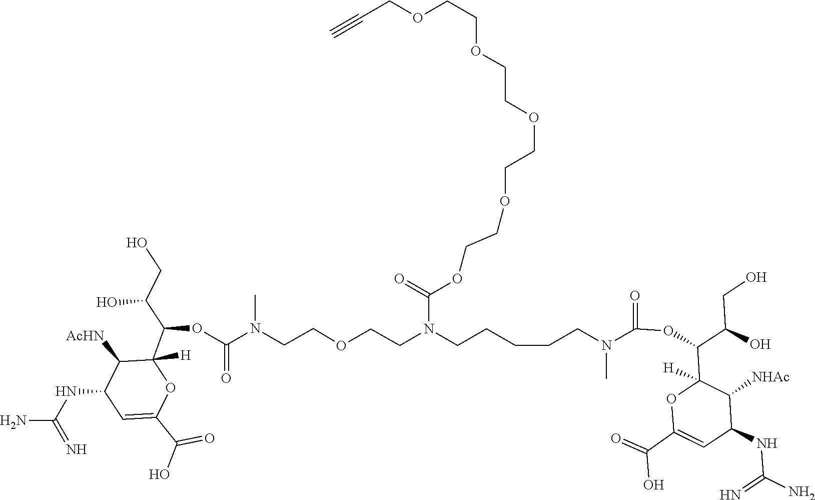 [SMILES: C#CCOCCOCCOCCOCCOC(=O)N(CCCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C- Inter- 99 | 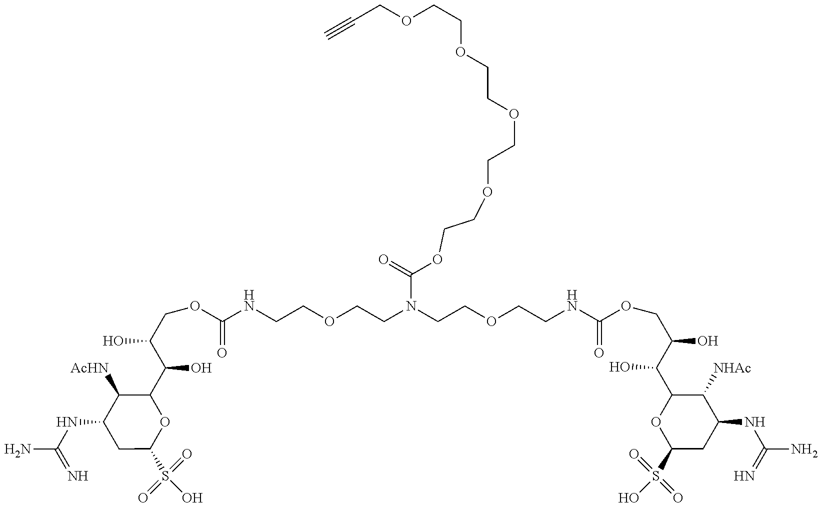 [SMILES: C#CCOCCOCCOCCOCCOC(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C- Inter- 100 | 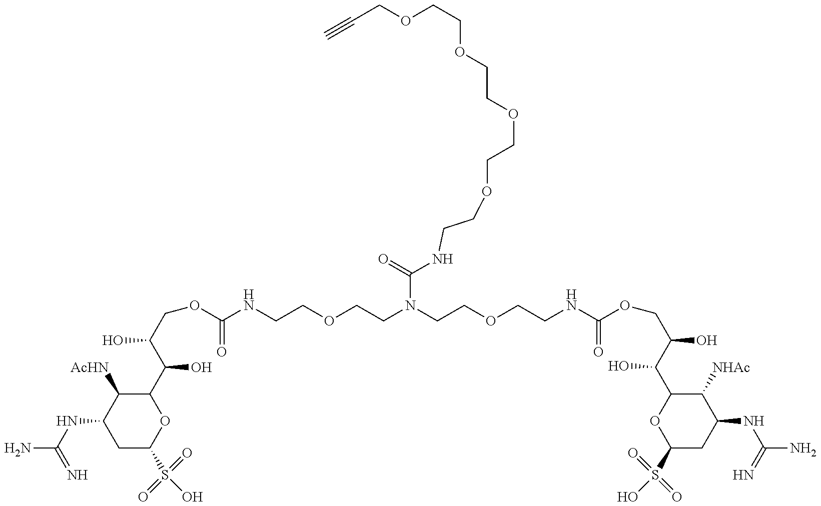 [SMILES: C#CCOCCOCCOCCOCCNC(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C- Inter- 101 | 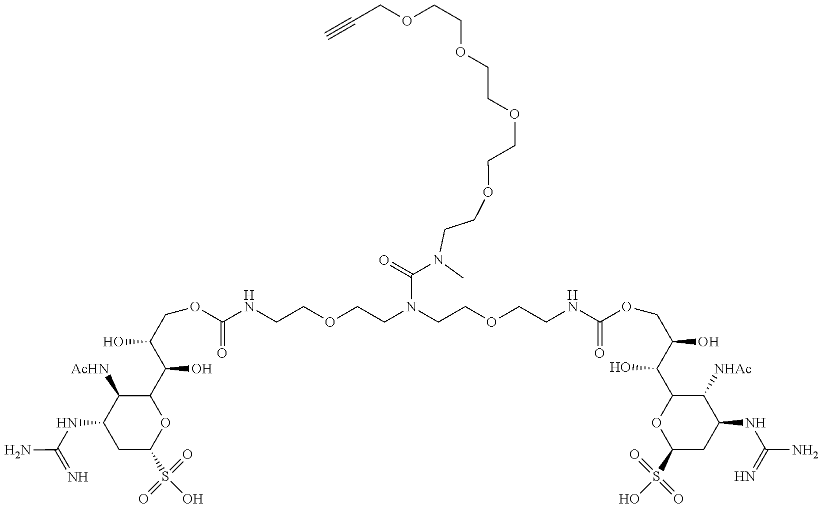 [SMILES: C#CCOCCOCCOCCOCCN(C)C(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C- Inter- 102 | 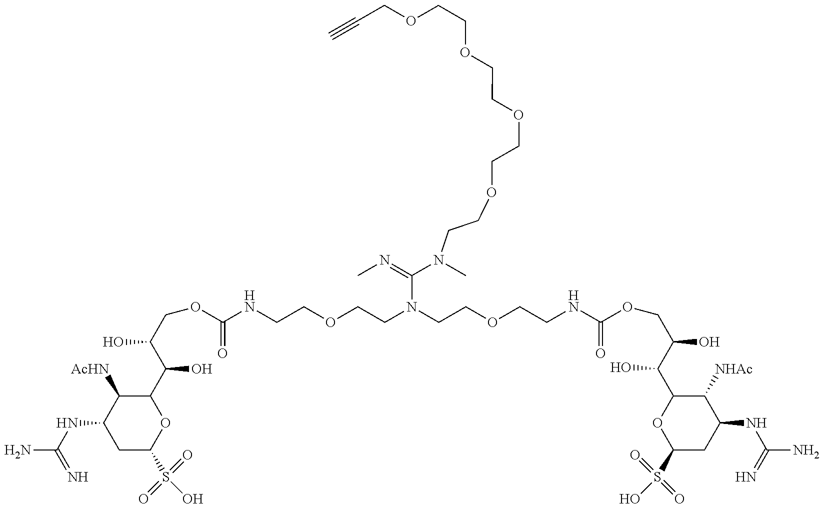 [SMILES: C#CCOCCOCCOCCOCCN(C)/C(=N/C)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C- Inter- 103 | 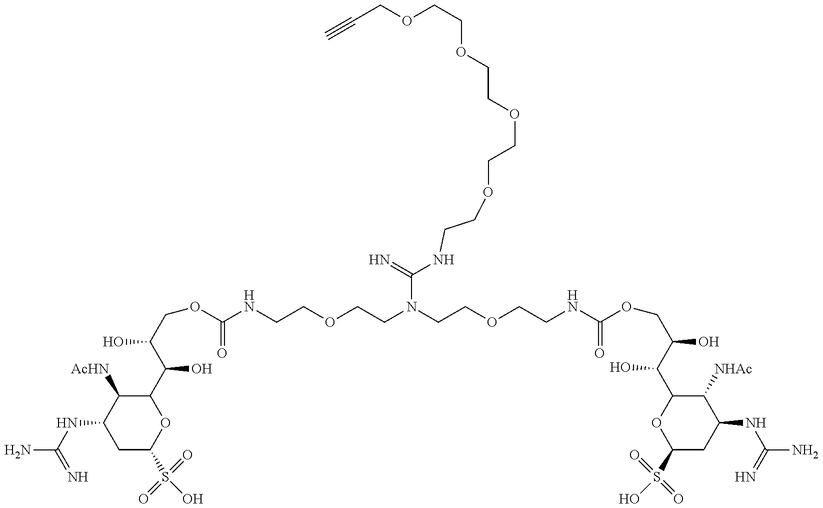 [SMILES: C#CCOCCOCCOCCOCCNC(=N)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C- Inter- 104 | 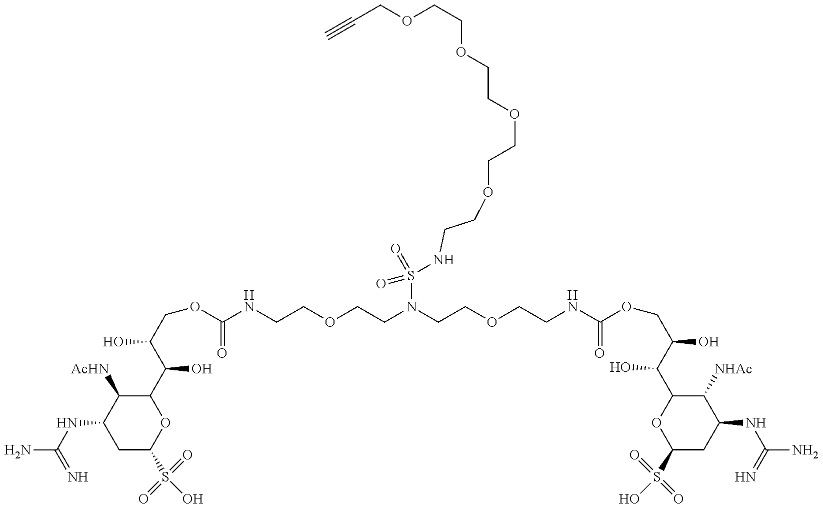 [SMILES: C#CCOCCOCCOCCOCCNS(=O)(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C- Inter- 105 | 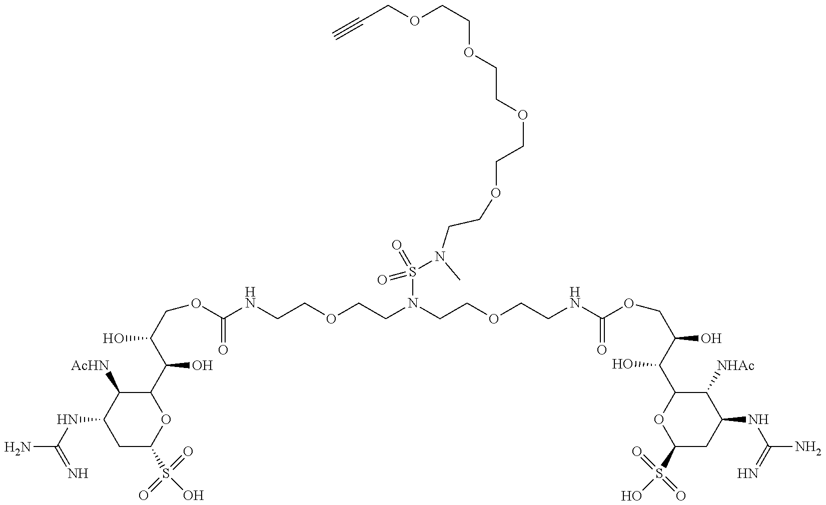 [SMILES: C#CCOCCOCCOCCOCCN(C)S(=O)(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C- Inter- 106 | 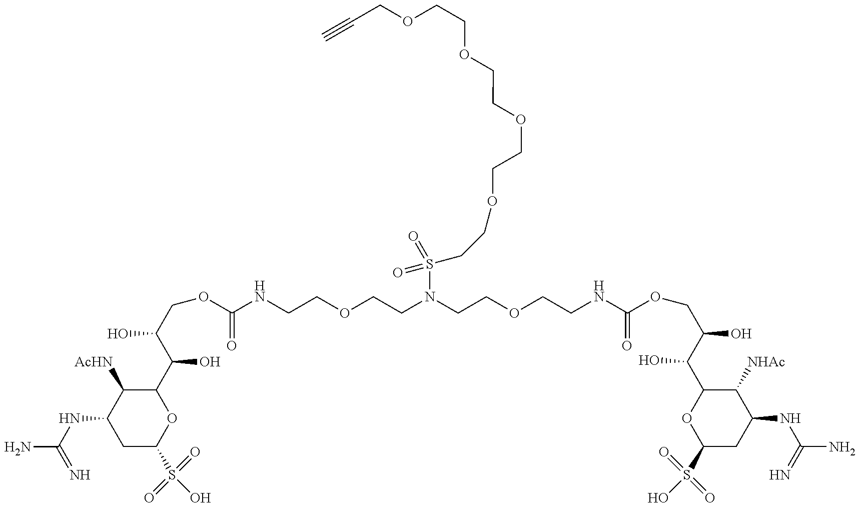 [SMILES: C#CCOCCOCCOCCOCCS(=O)(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C- Inter- 107 | 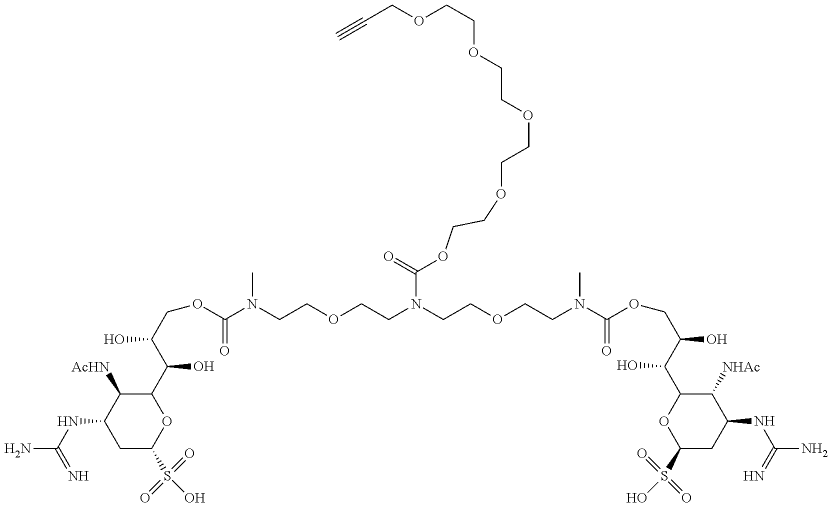 [SMILES: C#CCOCCOCCOCCOCCOC(=O)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C- Inter- 108 | 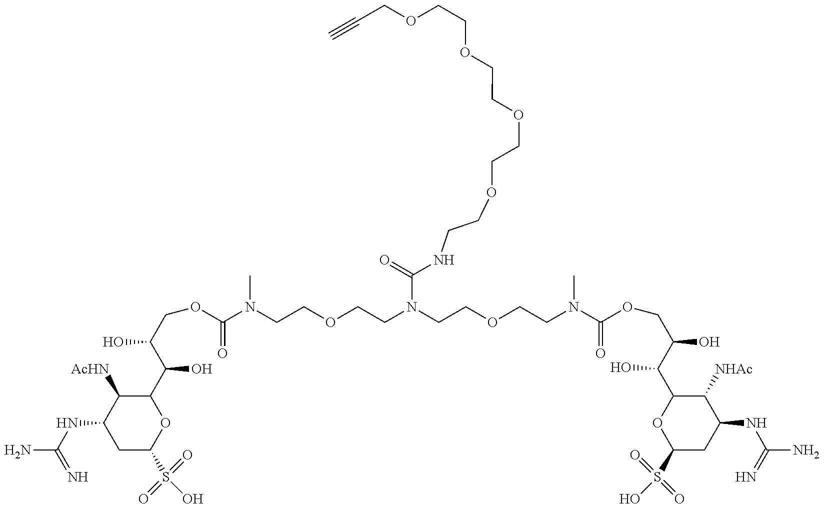 [SMILES: C#CCOCCOCCOCCOCCNC(=O)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C- Inter- 109 | 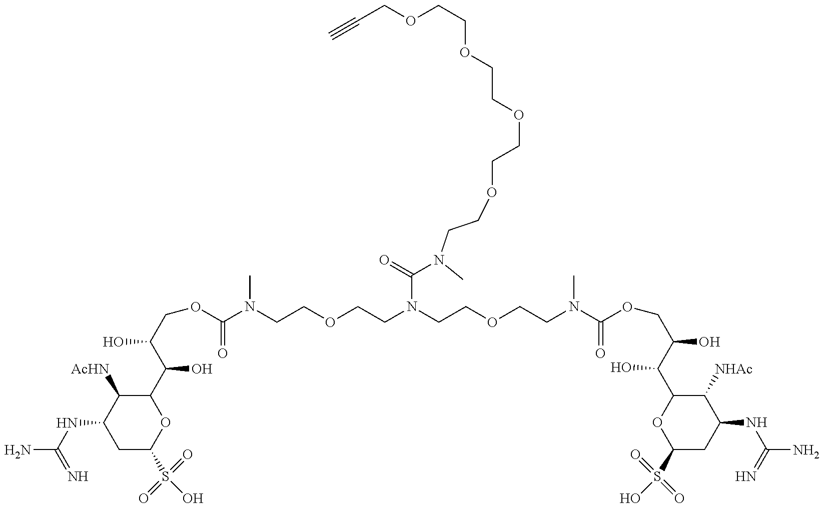 [SMILES: C#CCOCCOCCOCCOCCN(C)C(=O)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C- Inter- 110 | 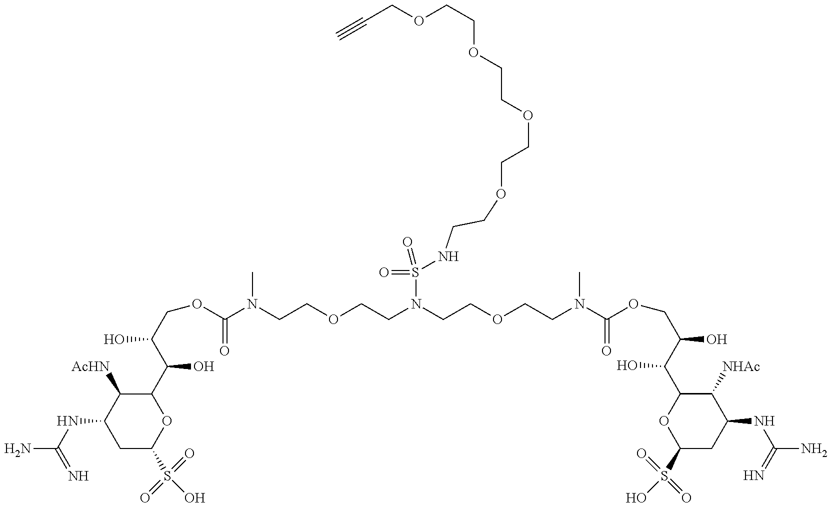 [SMILES: C#CCOCCOCCOCCOCCNS(=O)(=O)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C- Inter- 111 | 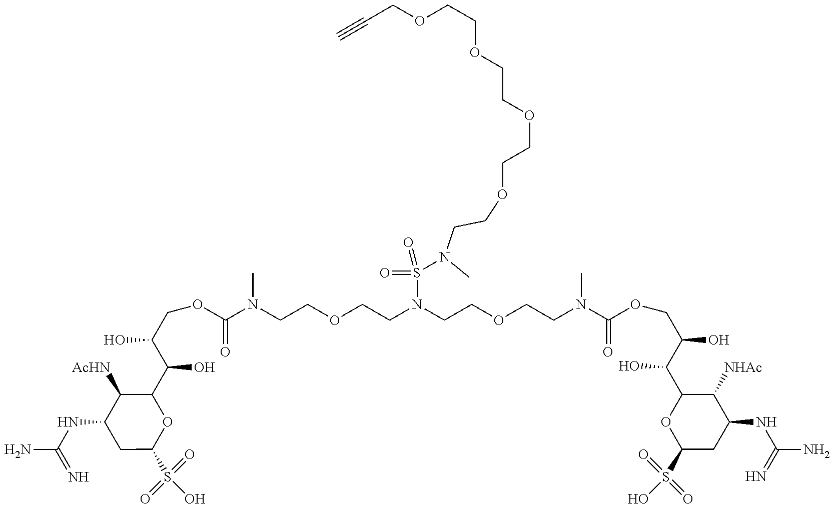 [SMILES: C#CCOCCOCCOCCOCCN(C)S(=O)(=O)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C- Inter- 112 | 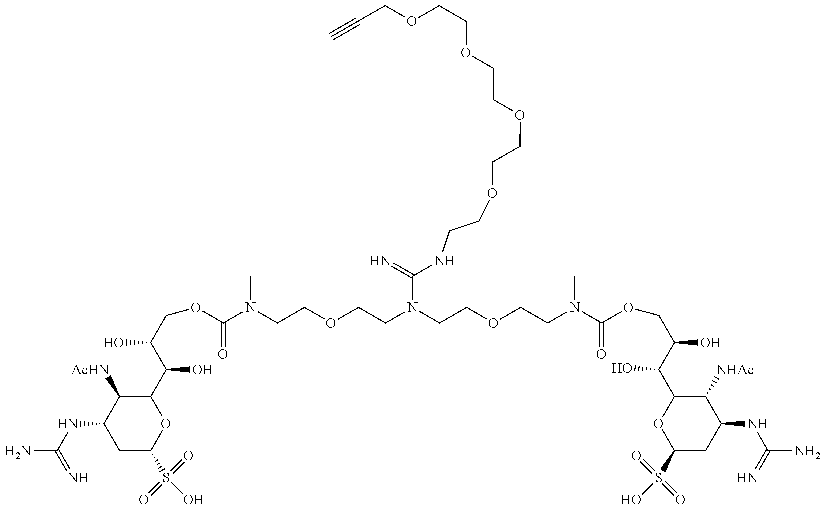 [SMILES: C#CCOCCOCCOCCOCCNC(=N)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C- Inter- 113 | 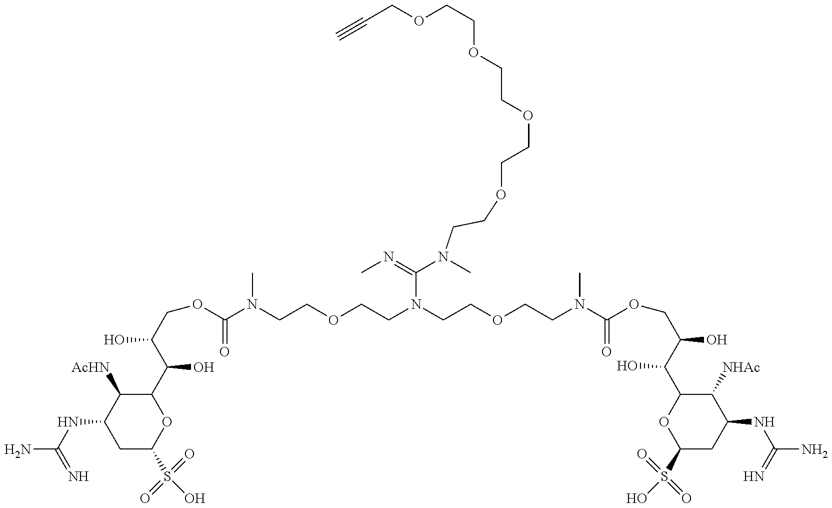 [SMILES: C#CCOCCOCCOCCOCCN(C)/C(=N/C)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C- Inter- 114 | 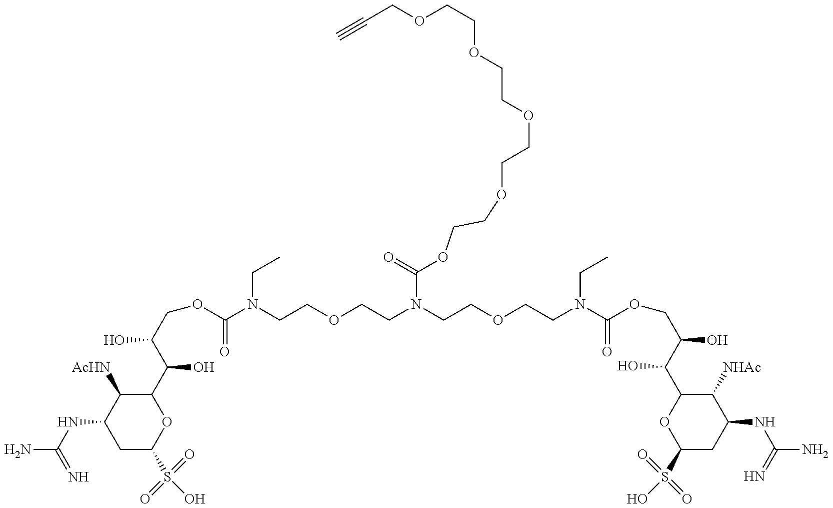 [SMILES: C#CCOCCOCCOCCOCCOC(=O)N(CCOCCN(CC)C(=O)OC[C@@H](O)[C@@H](O)C1O[C@H](S(=O)(=O)O)C[C@H](NC(=N)N)[C@H]1NC(C)=O)CCOCCN(CC)C(=O)OC[C@@H](O)[C@@H](O)C1O[C@H](S(=O)(=O)O)C[C@H](NC(=N)N)[C@H]1NC(C)=O] |
| C- Inter- 115 | 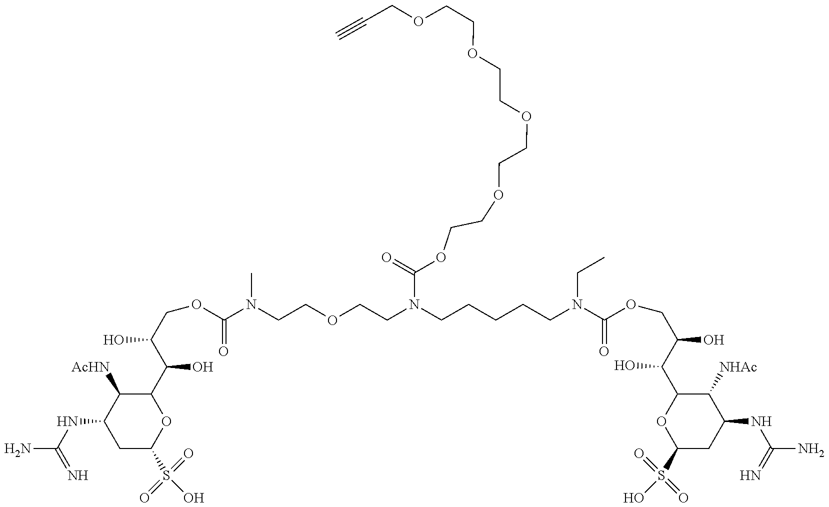 [SMILES: C#CCOCCOCCOCCOCCOC(=O)N(CCCCCN(CC)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |

In another aspect, provided herein is a pharmaceutical composition comprising the conjugate of formula I-1 as described above, or the compound of formula I-2 as described above, or a pharmaceutically acceptable salt, ester, isomer, solvate, prodrug or isotopically labeled derivative thereof, and optionally one or more other therapeutic agents, such as chemotherapeutic agents, angiogenesis inhibitors, cytokines, cytotoxic agents, other antibodies, other small molecule drugs or immunomodulators (e.g., immune checkpoint inhibitors or agonists), and optionally pharmaceutically acceptable excipients.

In another aspect, provided herein is a method for the prevention or treatment of a subject having a viral infection or at a risk of having a viral infection, comprising administering to the subject, for example by injection, an effective amount of the conjugate of formula I-1 as described above, or the compound of formula I-2 as described above, a pharmaceutically acceptable salt, ester, isomer, solvate, prodrug or isotopically labeled derivative thereof.

In another aspect, provided herein is a use of the conjugate of formula I-1 or the compound of formula I-2 as described above, a pharmaceutically acceptable salt, ester, isomer, solvate, prodrug or isotopically labeled derivative thereof, for the preparation of a medicament, wherein the medicament is used for preventing or treating a viral infection of a subject having a viral infection or at a risk of having a viral infection.

In some embodiments, the viral infection is an infection caused by an influenza virus or a parainfluenza virus.

In some embodiments, the viral infection is an infection caused by influenza virus A, B or C, or parainfluenza virus.

In some embodiments, the subject having a viral infection or at a risk of having a viral infection may be a subject with an immune system deficiency.

In some embodiments, the subject having a viral infection or at a risk of having a viral infection may be a subject who is or will be treated with an immunosuppressive agent.

In some embodiments, the subject having a viral infection or at a risk of having a viral infection may be a subject diagnosed with an immunosuppression disease.

In some embodiments, the subject diagnosed with an immunosuppression disease has cancer or acquired immunodeficiency syndrome;

In some embodiments, the subject diagnosed with an immunosuppression disease has leukemia, lymphoma, humoral immunodeficiency, T-cell deficiency, complement deficiency or multiple myeloma;

In some embodiments, the subject is a subject undergoing or about to undergo a hematopoietic stem cell transplant;

In some embodiments, the subject is a subject undergoing or about to undergo an organopoietic transplant; and/or

In some embodiments, the subject may be at a risk of a secondary infection.

In another aspect, provided herein is a method for preventing a secondary infection of a subject caused by an influenza virus infection, comprising administering to the subject, for example by an injection, an effective amount of the conjugate of formula I-1 as described above, or the compound of formula I-2 as described above, a pharmaceutically acceptable salt, ester, isomer, solvate, prodrug or isotopically labeled derivative thereof.

In some embodiments, said secondary infection is a respiratory infection.

In some embodiments, said secondary infection is associated with pneumonia.

In some embodiments, said secondary infection is a bacterial, or viral, or fungal infection.

In some embodiments, said bacterial infection is an infection caused by methicillin-resistant Staphylococcus aureus.

In some embodiments, said bacterial infection is an infection caused by Streptococcus pneumoniae.

In some embodiments, the conjugate of formula I-1 or the compound of formula I-2, or pharmaceutically acceptable salt, ester, isomer, solvate, prodrug or isotopically labeled derivative thereof, is administrated by an intramuscular injection, intravenous injection, intradermal injection, intra-arterial injection, intraperitoneal injection, intra-lesional injection, intracranial injection, intra-articular injection, intrapleural injection, intratracheal injection, intraprostatic injection, intranasal injection, intravitreous injection, intravaginal injection, intrarectal injection, local injection, intra-tumor injection, intraperitoneal injection, subcutaneous injection, subconjunctival injection, intracapsular injection, mucosal injection, intrapericardial injection, intra-umbilical injection, intra-ocular injection, oral, local inhalation, injection or infusion.

In some embodiments, the conjugate of formula I-1 or the compound of formula I-2, or pharmaceutically acceptable salt, ester, isomer, solvate, prodrug or isotopically labeled derivative thereof, combines with another therapeutic agent in administration or in the preparation of a medicament.

In some embodiments, another therapeutic agent is an antiviral drug.

In some embodiments, the antiviral drug is baloxavir, pimodivir, oseltamivir, zanamivir, peramivir, laninamivir, amantadine, MEDI8852 or rimantadine.

In some embodiments, another drug used by the subject is an antiviral vaccine.

In some embodiments, the antiviral drug and the conjugate of formula I-1 or the compound of formula I-2, or pharmaceutically acceptable salt, ester, isomer, solvate, prodrug or isotopically labeled derivative thereof, are sequentially administered, for example, by injection to the subject.

In some embodiments, the antiviral drug and the conjugate of formula I-1 or the compound of formula I-2, or pharmaceutically acceptable salt, ester, isomer, solvate, prodrug or isotopically labeled derivative thereof, are simultaneously administered, for example by injection, to the subject.

### Effects of the Disclosure

The conjugates of formula I-1 or compounds of formula I-2 in present disclosure, or pharmaceutically acceptable salts, esters, isomers, solvates, prodrugs or isotopic markers thereof, have the following advantages:
Possession of a significantly high antiviral activity
High activity against drug-resistant viral strains; and/or
Possession of the excellent in vitro/in vivo pharmacokinetic properties and safety, e.g., a long half-life, allowing the reduced dosing frequency and increased patient compliance with high clinical prospects.

### Preparations of Conjugates of Present Disclosure

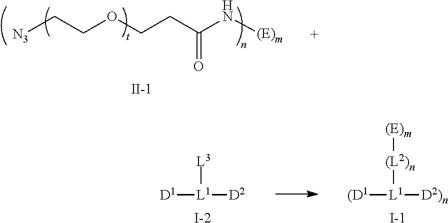

wherein each variable such as including D¹, D², E, L¹, L², L³, t, m and n are as defined above.

In one aspect, provided herein is a method for preparing conjugates of formula I-1, the method including:
Reacting compounds of formula II-1 (wherein each variable is as defined above)

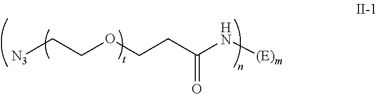

with compounds of formula I-2,

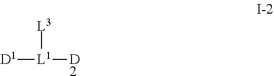

to obtain compounds of formula I-1.

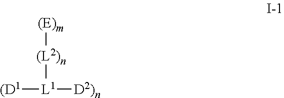

In some embodiments, compounds of formula II-1 were prepared by the following methods of:

Reacting compounds of formula II-2 (wherein t is as defined above (e.g. in definition of L²))

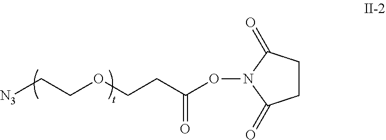

with (E)-NH₂ (wherein E is as defined above, —NH₂ is the amino of E),
to obtain compounds of formula II-1 (wherein each variable is as defined above)

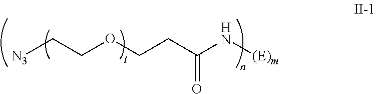

### Pharmaceutical Compositions

In some embodiments, the present disclosure provides pharmaceutical compositions comprising a conjugate of Formula I-1, a compound of Formula I-2, or a pharmaceutically acceptable salt, ester, isomer, solvate, prodrug or isotopically labeled derivative thereof. For the sake of simplicity, the conjugate of formula I-1, compound of formula I-2, or a pharmaceutically acceptable salt, ester, isomer, solvate, prodrug or isotopically labeled derivative thereof may be referred to as “compounds of present disclosure” in short. In one embodiment, said composition further includes a pharmaceutically acceptable excipient. In one embodiment, the pharmaceutical composition includes compounds of present disclosure, and its combination with one or more other therapeutic agents.

As used herein, “pharmaceutically acceptable excipients” include any and all physiologically compatible solvents, dispersing agents, isotonic agents, absorption delaying agents, etc.

For the use of pharmaceutically acceptable excipients, reference can be made in Handbook of Pharmaceutical Excipients, Eighth Edition, R. C. Rowe, P. J. Seskey and S. C. Owen, Pharmaceutical Press, London, Chicago. Pharmaceutical Press, London, Chicago.

The pharmaceutical compositions of the present disclosure may be in a variety of forms. These forms include, for example, liquid, semi-solid and solid forms, such as liquid solutions (e.g., injectable solutions and infusible solutions), bulk or suspension formulations, liposomal formulations and suppositories. The preferred form depends on the intended administration mode and therapeutic use.

The medicament including compounds of the present disclosure, preferably in the form of a lyophilized formulation or an aqueous solution, may be prepared by mixing compounds of the present disclosure having the desired purity with one or more optional pharmaceutically acceptable excipients.

### Definitions

The terms involved in the present disclosure are defined below. In addition, the following terms may be understood by those skilled in the art with the aid of the prior art. The undefined terms have the same meaning as commonly understood by those skilled in the art to which the present disclosure belongs.

The term “inhibits neuraminidase activity,” as used herein refers to an IC₅₀ of less than or equal to 1,000 nM, for example, as measured in accordance with the neuraminidase inhibition assay in Examples herein. In some embodiments, an IC₅₀ of less than or equal to 100 nM or less than or equal to 10 nM in accordance with neuraminidase inhibition activity is indicative of a compound inhibiting neuraminidase activity.

The term “inhibits viral growth”, as used herein refers to an EC₅₀ of less than or equal to 1000 nM. e.g., as measured in accordance with an experimental method for influenza virus-mediated cytopathic inhibitory activity assay in Examples herein. In some embodiments, a viral growth inhibitory activity of less than or equal to 100 nM or an IC50 of less than or equal to 10 nM is indicative of a compound inhibiting viral growth.

As used herein, the term “Fc structural domain monomer” refers to a polypeptide chain or a functional fragment thereof (e.g., a fragment capable of forming a dimer with another Fc structural domain monomer or capable of binding to an Fc receptor) having the following features that the polypeptide chain includes a second and a third antibody constant region (CH₂ and CH₃), and optional fourth antibody constant region. In some cases, the Fc structural domain monomer further includes at least one hinge region or a partial hinge region.

The Fc structural domain monomer may be of any immunoglobulin antibody type, including IgG, IgE, IgM, IgA, IgD. In addition, the Fc structural domain monomer may be of any IgG subtype (e.g., IgG1, IgG2a, IgG2b, IgG3, or IgG4). For example, in natural antibodies, the immunoglobulin Fc structural domain comprises: the second and third constant structural domains (CH₂ structural domains and CH3 structural domains) of the two heavy chains derived from the IgG, IgA, and IgD classes of antibodies; or the second, third, and fourth constant structural domains (CH₂ structural domains, CH₃ structural domains, and CH₄ structural domains) of the two heavy chains derived from the IgM and IgE classes of antibodies. Unless otherwise specified herein, numbering of amino acid residues in the IgG or Fc domain monomer is according to the EU numbering system for antibodies (which is also referred to as the Kabat EU, or EU numbering system, as described, in Kabat et al., Sequences of Proteins of Immunological Interest, 5th Ed. Public Health Service, National Institutes of Health, Bethesda, Md., 1991). The Fc domain monomers can be of any species origin, e.g., synthetic, human, mouse, rat, camelid, etc. The Fc domain monomers can contain one or more unnatural amino acid sequences that serve as targeted coupling sites for small molecule drugs. The Fc domain monomer may contain one or more solvent-exposed cysteine or lysine residues that have been site-modified to provide additional small molecule drug coupling sites.

As used herein, the term “Fc structural domain” refers to a dimer formed by the two Fc structural domain monomers interacting with each other through the hinge region, and/or the CH2, and/or CH3 antibody constant region. In some embodiments, the monomers of the dimer have one or more disulfide bonds. The Fc structural domain can be of any species origin, e.g., synthetic, human, mouse, rat, camelid, etc. The Fc structural domain may contain one or more unnatural amino acid sequences that serve as site-specific coupling sites for small molecule drugs. The Fc structural domain may contain one or more solvent-exposed cysteine or lysine residues that have been site-modified to provide additional small molecule drug coupling sites.

The term “covalently attached” as used herein refers to two portions of a conjugate that are connected to each other by a covalent bond, the covalent bond formed between two atoms in the two portions for connecting the conjugate.

As used herein, the term “Fc-binding peptide” refers to a polypeptide composed of 5 to 50 (e.g., 5 to 40, 5 to 30, 5 to 20, 5 to 15, 5 to 10, 10 to 50, 10 to 40, 10 to 30, or 10 to 20) consecutive amino acids and having an affinity for and function in binding to an Fc structural domain (e.g., any Fc structural domain described herein). The Fc binding peptide can be of any species origin, e.g., synthetic, human, mouse, rat, camelid, etc. The Fc binding peptide may contain one or more unnatural amino acid sequences that serve as site-specific coupling sites for small molecule drugs. The Fc binding peptide may contain one or more solvent-exposed cysteine or lysine residues that have been site-modified to provide additional small molecule drug coupling sites.

The term “solvent-exposed” as used herein refers to an amino acid residue surrounded by a solvent molecule. The solvent-exposed amino acid molecule may be formed naturally or artificially (including both natural and unnatural amino acids). In some embodiments, the site modification does not affect the spatial three-dimensional structure of the protein.

As used herein, the term “percent (%) identity” refers to the percentage of amino acid residues of a candidate sequence, e.g., an Fc-IgG, or fragment thereof, that are identical to the amino acid residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software.

As used herein, the term “treatment” or “to treat” refers to therapeutic treatment of an individual infected with a virus. In some embodiments, the therapeutic treatment may slow the progression of the viral infection, reduce the symptoms of the individual, and/or eliminate the viral infection.

The term “effective amount” or “effective dose” refers to such an amount or dose of an antibody or fragment or composition or combination of the present disclosure that, when administered to a patient in a single or multiple doses, produces the desired effect in the patient in need of the treatment or prevention.

The term “C₁-C₂₀ alkyl” (e.g. C₁-C₂₀ alkyl aryl), alone or in combination, represents a saturated straight or branched chain containing 1-20, e.g. 1-15, 1-10 (e.g. 1, 2, 3, 4, 5, 6, 7, 8, 9, or 10 carbon atoms), and in particular 1-6 carbon atoms, which includes methyl, ethyl, propyl, isopropyl, butyl, sec-butyl, isobutyl, tert-butyl, n-pentyl, 2-pentyl, 3-pentyl, 2-methyl-2-butyl, 3-methyl-2-butyl, 3-methyl-1-butyl, 2-methyl-1-butyl, n-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3,-dimethyl-2-butyl and the like. Preferably, “C₁₋₂₀ alkyl” is any of methyl, ethyl, isopropyl and tert-butyl.

The term “C₂-C₂₀ alkenyl” represents a straight or branched alkyl group as defined above, which contains one or more, e.g., 1-10, e.g., 1, 2, 3, 4, or 5 alkenyl, and not contains an alkynyl group. Exemplary chain alkenyl groups include vinyl, propenyl, iso-propenyl, butenyl, sec-butenyl, iso-butenyl, n-pentenyl, 2-pentenyl, 3-pentenyl, 2-methyl-2-butenyl, 3-methyl-2-butenyl, 3-methyl-1-butenyl, 2-methyl-1-butenyl, n-hexenyl, 2-hexenyl, 3-hexenyl, 2-methyl-2-pentenyl, and the like.

The term “C₃₋₂₀ cycloalkyl” (e.g., C3-C20 cycloalkyl aryl), alone or in combination, represents a saturated cycloalkyl group having 3-20, e.g., 3-15, 3-10 (e.g., 3, 4, 5, 6, 7, 8, 9, or 10 carbon atoms), and in particular 3-6 carbon atoms, including cyclo-propyl, cyclo-butyl, cyclo-pentyl, cyclohexyl, cycloheptyl, and the like. In particular, “C₃₋₇ cycloalkyl” is cyclopropyl, cyclopentyl, cyclohexyl and the like.

The term “C₃-C₂₀ cycloalkenyl” denotes a cycloalkyl group having 3-20, e.g. 3-15, 3-10 (e.g. 3, 4, 5, 6, 7, 8, 9 or 10 carbon atoms), in particular 3-6 carbon atoms, which includes one or more, e.g. 1-10, e.g. 1, 2, 3, 4 or 5 alkenyl bonds, and not include an alkynyl group and is not an aromatic group. Exemplary cycloalkenyl groups include cyclopropenyl, cyclobutenyl, cyclopentenyl, cyclohexenyl, cycloheptenyl, and the like. In particular, “C₃₋₇ cycloalkenyl” is cyclopropenyl, cyclopentenyl, cyclohexenyl and the like.

The term “halogen”, alone or in combination, denotes fluorine, chlorine, bromine or iodine, in particular fluorine, chlorine or bromine.

The term “alkyl halide”, alone or in combination, denotes an alkyl group as defined above substituted with one or more (e.g. 1, 2, 3, 4 or 5) halogens.

The term “amino”, alone or in combination, denotes primary (—NH₂), secondary (—NH—) or tertiary (—NH—).

The term “carbonyl”, also known as “—C(═O)—”, refers to a divalent group formed by only one carbon atom and one oxygen atom connected with each other by a double bond, the carbon atom thereof connected to the other two fragments by a single bond.

The term “heterocycloalkyl”, refers to a saturated or partially unsaturated (including 1 or 2 double bonds) non-aromatic cyclic group consisting of a carbon atom and a heteroatom such as nitrogen, oxygen or sulfur. The cyclic group may be a monocyclic or bicyclic group, and in the context of the present disclosure may be a 3- to 20-membered heterocycloalkyl such as 3- to 15-membered, 3- to 10-membered, 3- to 7-membered, 3- to 6-membered, 5- to 7-membered, or 4- to 6-membered heterocycloalkyl group. The number of carbon atoms in the heterocycloalkyl group may be 2-16, for example, 2-11. The number of heteroatoms may be 1 or more, preferably 1, 2, 3 or 4. The nitrogen, carbon or sulfur atoms in the heterocycloalkyl group may optionally be oxidized. The hydrogen atom on the “heterocycloalkyl” is independently and optionally substituted with one or more of the substituents described in present disclosure. The “heterocycloalkyl” can be linked to the parent molecule by any one of the ring atoms on the ring. The terms “3- to 6-membered heterocycloalkyl” and “3- to 7-membered heterocycloalkyl” refer to saturated or partially unsaturated monocyclic or polycyclic heterocycloalkyl groups including 3 to 6 and 3 to 7 of ring members (selected from carbon atoms and heteroatoms or heteroatom groups), respectively. The heteroatoms or heteroatom groups is selected from N, O, S(O)m (wherein m is any integer from 0 to 2), for example, acridinyl, acridinyl, oxetidinyl, tetrahydropyrrolyl, oxopyrrolidinyl, tetrahydrofuryl, tetrahydrothiophenyl, piperidinyl, morpholinyl, piperazinyl, thiomorpholinyl, tetrahydropyranyl, 1,1-dioxidothiomorpholinyl, and the like.

The term “aryl” denotes any stable 6- to 15-membered, e.g. 6- to 10-membered, monocyclic or bicyclic aromatic carbocyclic hydrocarbon group, including phenyl, naphthyl, tetrahydronaphthyl, 2,3-dihydronaphthyl, or biphenyl. The hydrogen atom on the “aryl” group is independently and optionally substituted with one or more of the substituents described in present disclosure.

The term “heteroaryl” or “heterocyclic aryl” denotes an aromatic ring group formed by the substitution of a carbon atom on the ring by at least one heteroatom selected from sulfur, oxygen or nitrogen. The aromatic ring group may be a 5- to 15-membered ring, such as a 5- to 7-membered or 5- to 6-membered monocyclic or 7- to 12 bicyclic group, including but not limited to, 5-, 6-, 7-, 8-, 9- or 12-membered heteroaryl group. In the present disclosure, the number of heteroatoms in the heteroaryl group is preferably 1, 2, 3 or 4. The exemplary heteroaryl groups are selected, for example, from thienyl, pyridyl, pyrimidinyl, pyrazinyl, pyridazinyl, pyridin-2(1H)-one, pyridin-4(1H)-one, pyrroloxy, pyrazolyl, thiazolyl, 1,2,3-triazolyl, 1,2,4-triazolyl, imidazolyl, tetrazolyl, isothiazolyl, oxazolyl, iso oxazolyl, thiadiazolyl, oxadiazolyl, benzothienyl, indolyl, benzimidazolyl, benzothiazolyl, benzofuranyl, quinolinyl, isoquinolinyl, quinazolinyl, and so on. The hydrogen atom on the “heteroaryl” group is independently and optionally replaced by one or more of the substituents described in present disclosure.

The term “C₆₋₁₅ aryl” denotes an aryl group having 6 to 15 carbon atoms, where “aryl” is as defined above.

The term “5- to 15-membered heteroaryl” denotes a heteroaryl group having 5- to 15 ring atoms, wherein “heteroaryl” is as defined above.

The term “cyano” denotes the group —CN.

The term “carboxyl” denotes the group —COOH.

The term “hydroxyl” denotes the group —OH.

The term “substituted” means having one or more substituents such as 1-25, 1-20, 1-10, or 1-5 substituents, e.g., 1, 2, 3, 4, 5 substituents. Substituents include, but are not limited to, alkyl, cycloalkyl, alkenyl, alkynyl, aryl, alkaryl, acyl, heteroaryl, heteroalkyl, heterocycloalkyl, heteroalkenyl, heteroalkynyl, heteroalkylaryl, halogen, oxo, cyano, nitro, amino, alkylamino, hydroxy, alkoxy, alkanoyl, carbonyl, carbamoyl, guanidinium, ureido, amidinium, and combinations of the foregoing groups or portions. Substituents include, but are not limited to, F, Cl, methyl, phenyl, phenylmethyl, OR, NR₂, SR, SOR, SO₂R, OCOR, NRCOR, NRCONR₂, NRCOOR, OCONR₂, RCO, COOR alkyl-OOCR, SO₃R, CONR₂, SO₂NR₂, NRSO₂NR₂, CN, CF₃, OCF₃, SiR₃, and NO₂, wherein R is each independently H, alkyl, alkenyl, aryl, heteroalkyl, heteroalkenyl, or heteroaryl, and wherein two optionally present substituents on the same or adjacent atoms may be linked to form a 3- to 8-membered fused and optionally-substituted, aryl or non-aryl, saturated or unsaturated ring, or wherein two optionally present substituents on the same atom may be linked to form a 3- to 8-membered and optionally substituted aromatic or non-aromatic, saturated or unsaturated ring.

The term “optionally” indicates the subsequent events or circumstances, etc., may occur/exist or not occur/not exist. For example, the term “optionally substituted” indicates that the involved group may or may not be substituted.

The term “comprise” “include” also covers situations completely or essentially consisted of the involved features or elements.

The term “stereoisomer” encompasses all tautomeric forms, including enantiomers, diastereomers, and geometric isomers (cis-trans isomers). Accordingly, individual stereochemical isomers of the compounds, or enantiomers, diastereomers, geometrical isomers (or cis-trans isomers) or the mixture thereof, fall within the scope of present disclosure.

The term “pharmaceutically acceptable salt” indicates that the compounds of present disclosure are present in the form of their pharmaceutical salts, including acid addition salts and base addition salts. In present disclosure, pharmaceutically acceptable, non-toxic acid addition salts denote salts formed by the compounds of present disclosure with organic or inorganic acids. The organic or inorganic acids include but are not limited to, hydrochloric acid, sulfuric acid, hydrobromic acid, hydriodic acid, phosphoric acid, nitric acid, perchloric acid, acetic acid, oxalic acid, maleic acid, fumaric acid, tartaric acid, benzene sulphonic acid, tartaric acid, methanesulphonic acid, salicylic acid, succinic acid, citric acid, lactic acid, propanoic acid, benzoic acid, p-toluene sulfonic acid, malic acid and the like. The pharmaceutically acceptable, non-toxic base addition salts denote salts formed by the compounds of present disclosure with organic or inorganic bases. The organic or inorganic bases include but are not limited to, alkali metal salts, such as lithium, sodium, or potassium salts; alkaline earth metal salts, such as calcium or magnesium salts; and organic alkali salts, such as ammonium salts or N+(C₁₋₆ alkyl)₄ salts, formed with the organic bases containing an N group.

The term “solvate” denotes the complex formed by one or more solvent molecules and the compounds of present disclosure. Solvents that form solvates include, but are not limited to, water, methanol, ethanol, isopropanol, ethyl acetate, tetrahydrofuran, N, N-dimethylformamide, dimethyl sulfoxide, and the like.

The term “hydrate” refers to complex formed by water and the compounds of present disclosure.

The term “prodrug” denotes chemical derivatives of the compounds of present disclosure which is converted in vivo to a compound of general formula I-1 or I-2 by a chemical reaction.

The term “isotopically labeled derivative” denotes the isotopically labeled derivative obtained by substituting one or more, e.g., 1, 2, 3, 4, or 5 hydrogen atoms of formula I-1 and formula I-2 with deuterium atoms, or the isotopically labeled derivative obtained by substituting the carbon atoms of formula I-1 and formula I-2 with one or more, e.g., 1-3 of carbon 14 atoms (¹⁴C).

The term “drug:antibody ratio” or “DAR” refers to the ratio of the small molecule drug portion (D¹ and D²) linked to the E portion (e.g., antibody, Fc domain, or albumin) described herein to the E portion. In some embodiments described herein, the DAR is represented by n:m in formula I-1, being from 1 to 20, preferably from 2 to 10, such as 2, 3, 4, 5, 6, 7, 8, 9, or 10. The DAR can also be calculated as the average DAR of the overall molecules in the product, i.e., the overall ratio of small molecule drug portion (D¹ and D²) linked to E portion (e.g., the antibody, Fc structural domain or albumin) described herein to the E portion, measured by an assay method (e.g., by an MS method). Such DAR is referred to in the text as the average DAR. In some embodiments, the average DAR of the conjugate of the present disclosure is between 0.5 and 10.0, such as 1.0-8.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0, or the range with two of these values as the endpoints.

### SEQ ID NOs: 1-68 and Description Thereof

SEQ ID NO: 1: murine Fc-IgG2a with addition of IL2 signaling sequence (bold) at the N-
terminus
MYRMQLLSCIALSLALVTNSPRGPTIKPCPPCKCPAPNLLGGPSVFIFPPKIKDVLMIS
LSPIVTCVVVDVSEDDPDVQISWFVNNVEVHTAQTQTHREDYNSTLRVVSALPIQHQ
DWMSGKEFKCKVNNKDLPAPIERTISKPKGSVRAPQVYVLPPPEEEMTKKQVTLTCM
VTDFMPEDIYVEWTNNGKTELNYKNTEPVLDSDGSYFMYSKLRVEKKNWVERNSY
SCSVVHEGLHNHHTTKSFSRTPGK
SEQ ID NO: 2: Mature murine Fc-IgG2a
PRGPTIKPCPPCKCPAPNLLGGPSVFIFPPKIKDVLMISLSPIVTCVVVDVSEDDPDVQIS
WFVNNVEVHTAQTQTHREDYNSTLRVVSALPIQHQDWMSGKEFKCKVNNKDLPAPI
ERTISKPKGSVRAPQVYVLPPPEEEMTKKQVTLTCMVTDFMPEDIYVEWTNNGKTEL
NYKNTEPVLDSDGSYFMYSKLRVEKKNWVERNSYSCSVVHEGLHNHHTTKSFSRTP
GK
SEQ ID NO: 3: Human Fc-IgG1 with additions of IL2 signaling sequence (bold) and MVRS
amino acid sequence (underlined) at the N-terminus
MYRMQLLSCIALSLALVTNSMVRSDKTHTCPPCPAPELLGGPSVFLFPPKPKDTLMI
SRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVF
SCSVMHEALHNHYTQKSLSLSPGK
SEQ ID NO: 4: Mature human Fc-IgG1 with addition of MVRS amino acid sequence
(underlined) at the N-terminus
MVRSDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLS
PGK
SEQ ID NO: 5: Murine Fc-IgG2a with additions of IL2 signaling sequence (bold) at the N-
terminus and six histidine tags (italic) at the C-terminus
MYRMQLLSCIALSLALVTNSPRGPTIKPCPPCKCPAPNLLGGPSVFIFPPKIKDVLMIS
LSPIVTCVVVDVSEDDPDVQISWFVNNVEVHTAQTQTHREDYNSTLRVVSALPIQHQ
DWMSGKEFKCKVNNKDLPAPIERTISKPKGSVRAPQVYVLPPPEEEMTKKQVTLTCM
VTDFMPEDIYVEWTNNGKTELNYKNTEPVLDSDGSYFMYSKLRVEKKNWVERNSY
SCSVVHEGLHNHHTTKSFSRTPGKHHHHHH
SEQ ID NO: 6: Mature murine Fc-IgG2a with additions of six histidine tags (italic) at the C-
terminus
PRGPTIKPCPPCKCPAPNLLGGPSVFIFPPKIKDVLMISLSPIVTCVVVDVSEDDPDVQIS
WFVNNVEVHTAQTQTHREDYNSTLRVVSALPIQHQDWMSGKEFKCKVNNKDLPAPI
ERTISKPKGSVRAPQVYVLPPPEEEMTKKQVTLTCMVTDFMPEDIYVEWTNNGKTEL
NYKNTEPVLDSDGSYFMYSKLRVEKKNWVERNSYSCSVVHEGLHNHHTTKSFSRTP
GKHHHHHH
SEQ ID NO: 7: Human Fc-IgG1 with additions of IL2 signaling sequence (bold) and MVRS
amino acid sequence (underlined) at the N-terminus and six histidine tags (italic) at the C-
terminus
MYRMQLLSCIALSLALVTNSMVRSDKTHTCPPCPAPELLGGPSVFLFPPKPKDTLMI
SRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVF
SCSVMHEALHNHYTQKSLSLSPGKHHHHHH
SEQ ID NO: 8: Human mature Fc-IgG1 with additions of MVRS amino acid sequence at the
N-terminus (underlined) and six histidine tags at the C-terminus (italic)
MVRSDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLS
PGKHHHHHH
SEQ ID NO: 9: Human Fc-IgG1 with addition of IL2 signaling sequence (bold) and MVRS
amino acid sequence (underlined) at the N-terminus, two additional cysteines (*) in the hinge
region and additions of six histidine tags (italic) at the C-terminus
MYRMQLLSCIALSLALVTNSMVRSDKTHTCPPCPPC*KC*PAPELLGGPSVFLFPPKP
KDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRW
QQGNVFSCSVMHEALHNHYTQKSLSLSPGKHHHHHH
SEQ ID NO: 10: Mature human Fc-IgG1 with addition of MVRS amino acid sequence
(underlined) at the N-terminus (underlined), two additional cysteines (*) in the hinge
region and additions of six histidine tags (italic) at the C-terminus
MVRSDKTHTCPPCPPC*KC*PAPELLGGPSVFLFPPKPKDTLMISRTPEVTCVVVDVSH
EDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHY
TQKSLSLSPGKHHHHHH
SEQ ID NO: 11: Mature human Fc-IgG1 with addition of MVRS amino acid sequence
(underlined) at the N-terminus and two additional cysteines (*) in the hinge region
MVRSDKTHTCPPCPPC*KC*PAPELLGGPSVFLFPPKPKDTLMISRTPEVTCVVVDVSH
EDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQMQGNVFSCSVMHEALHNH
YTQKSLSLSPGK
SEQ ID NO: 12: Murine Fc-IgG2a with addition of IL2 signaling sequence (bold) at the N-
terminus and substitution (*) from Asn to Ala and additions of ix histidine tags (italic)
at the C-terminus
MYRMQLLSCIALSLALVTNSPRGPTIKPCPPCKCPAPNLLGGPSVFIFPPKIKDVLMIS
LSPIVTCVVVDVSEDDPDVQISWFVNNVEVHTAQTQTHREDYA*STLRVVSALPIQHQ
DWMSGKEFKCKVNNKDLPAPIERTISKPKGSVRAPQVYVLPPPEEEMTKKQVTLTCM
VTDFMPEDIYVEWTNNGKTELNYKNTEPVLDSDGSYFMYSKLRVEKKNWVERNSY
SCSVVHEGLHNHHTTKSFSRTPGKHHHHHH
SEQ ID NO: 13: Murine Fc-IgG2a with substitution (*) from Asn to Ala and additions of six
histidine tags (italic) at the C-terminus
PRGPTIKPCPPCKCPAPNLLGGPSVFIFPPKIKDVLMISLSPIVTCVVVDVSEDDPDVQIS
WFVNNVEVHTAQTQTHREDYA*STLRVVSALPIQHQDWMSGKEFKCKVNNKDLPAP
IERTISKPKGSVRAPQVYVLPPPEEEMTKKQVTLTCMVTDFMPEDIYVEWTNNGKTE
LNYKNTEPVLDSDGSYFMYSKLRVEKKNWVERNSYSCSVVHEGLHNHHTTKSFSRT
PGKHHHHHH
SEQ ID NO: 14: Human Fc-IgG1 with additions of IL2 signaling sequence (bold) and MVRS
amino acid sequence (underlined) at the N-terminus, substitution (*) from Asn to Ala and
additions of six histidine tags (italic) at the C-terminus
MYRMQLLSCIALSLALVTNSMVRSDKTHTCPPCPAPELLGGPSVFLFPPKPKDTLMI
SRTPEVTCVVVDVSHEDPEVKFNWYVDGVEVHNAKTKPREEQYA*STYRVVSVLTV
LHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNV
FSCSVMHEALHNHYTQKSLSLSPGKHHHHHH
SEQ ID NO: 15: Human Fc-IgG1 with addition of MVRS amino acid sequence (underlined) at
the N-terminus, substitution (*) from Asn to Ala and additions of six histidine tags (italic)
at the C-terminus
MVRSDKTHTCPPCPAPELLGGPSVFLFPPKPKDTLMISRTPEVTCVVVDVSHEDPEVK
FNWYVDGVEVHNAKTKPREEQYA*STYRVVSVLTVLHQDWLNGKEYKCKVSNKAL
PAPIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLS
PGKHHHHHH
SEQ ID NO: 16: Human Fc-IgG1 with additions of human serum albumin signal sequence
(bold) and ISAMVRS amino acid sequence (underlined) at the N-terminus
MKWVTFISLLFLFSSAYSISAMVRSDKTHTCPPCPAPELLGGPSVFLFPPKPKDTLMIS
RTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFS
CSVMHEALHNHYTQKSLSLSPGK
SEQ ID NO: 17: Human Fc-IgG1 with additions of human serum albumin signaling sequence
(bold) and ISAMVRS amino acid series (underlined) at the N-terminus, and containing C-
terminal G4S connector (italic) and C-terminal c-Myc tag (underlined, italic)
MKWVTFISLLFLFSSAYSISAMVRSDKTHTCPPCPAPELLGGPSVFLFPPKPKDTLMIS
RTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFS
CSVMHEALHNHYTQKSLSLSPGGGGGSEQKLISEEDL
SEQ ID NO: 18: Mature human Fc-IgG1 with addition of ISAMVRS amino acid sequence
(underlined) at the N-terminus, and additions of the G4S connector (italic) and c-Myc
tag(underlined, italic) at the C-terminus
ISAMVRSDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSL
SLSPGGGGGSEQKLISEEDL
SEQ ID NO: 19: human Fc-IgG1 with additions of human serum albumin signal sequence
(bold) and ISAMVRS amino acid sequence (underlined) at the N-terminus and alteration (*)
from lysine to serine to prevent coupling at such a site
MKWVTFISLLFLFSSAYSISAMVRSDKTHTCPPCPAPELLGGPSVFLFPPKPS*DTLMI
SRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVF
SCSVMHEALHNHYTQKSLSLSPGK
SEQ ID NO: 20: Mature human Fc-IgG1 with addition of the ISAMVRS amino acid sequence
(underlined) at the N-terminus and alteration (*) from lysine to serine to prevent coupling
at this site
ISAMVRSDKTHTCPPCPAPELLGGPSVFLFPPKPS*DTLMISRTPEVTCVVVDVSHEDP
EVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKS
LSLSPGK
SEQ ID NO: 21: Human Fc-IgG1 with additions of human serum albumin signal sequence
(bold) and ISAMVRS amino acid sequence (underlined) at the N-terminus, alteration (*) from
lysine to serine to prevent coupling at this site, and additions of a G4S connector (italic)
and a c-Myc tag (underlined, italic) at the C-terminus
MKWVTFISLLFLFSSAYSISAMVRSDKTHTCPPCPAPELLGGPSVFLFPPKPS(*)DTL
MISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGN
VFSCSVMHEALHNHYTQKSLSLSPGGGGGSEQKLISEEDL
SEQ ID NO: 22: Mature human Fc-IgG1 with addition of the ISAMVRS amino acid sequence
(underlined) at the N-terminus, alteration (*) from lysine to serine to prevent coupling at
this site, and additions of a G4S connector (italic) and a c-Myc tag (underlined, italic) at
the C-terminus
ISAMVRSDKTHTCPPCPAPELLGGPSVFLFPPKPS(*)DTLMISRTPEVTCVVVDVSHED
PEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKS
LSLSPGGGGGSEQKLISEEDL
SEQ ID NO: 23: Human Fc-IgG1 with additions of human serum albumin signal sequence
(bold) and ISAMVRS amino acid sequence (underlined) at the N-terminus, alteration (*) from
lysine to serine, and additions of a G4S connector (italic) and a c-Myc tag (underlined,
italic) at the C-terminus
MKWVTFISLLFLFSSAYSISAMVRSDKTHTCPPCPAPELLGGPSVFLFPPKPKDTLMIS
RTPEVTCVVVDVSHEDPEVKFNWYVDGVEVHNAKTKPREEQYA(*)STYRVVSVLTV
LHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNV
FSCSVMHEALHNHYTQKSLSLSPGGGGGSEQKLISEEDL
SEQ ID NO: 24: Mature human Fc-IgG1 with addition of ISAMVRS amino acid sequence
(underlined) at the N-terminus, alteration (*) from lysine to serine and additions of a G4S
connector (italic) and a c-Myc tag (underlined, italic) at the C-terminus
ISAMVRSDKTHTCPPCPAPELLGGPSVFLFPPKPKDTLMISRTPEVTCVVVDVSHEDPE
VKFNWYVDGVEVHNAKTKPREEQYA(*)STYRVVSVLTVLHQDWLNGKEYKCKVSN
KALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKS
LSLSPGGGGGSEQKLISEEDL
SEQ ID NO: 25: Human Fc-IgG1 with additions of human serum albumin signal sequence
(bold) and ISAMVRS amino acid sequence (underlined) at the N-terminus, alterations of
H310A(*) and H435A(*) to prevent FcRn binding, and additions of a G4S connector (italic)
and a c-Myc tag (underlined, italic) at the C-terminus
MKWVTFISLLFLFSSAYSISAMVRSDKTHTCPPCPAPELLGGPSVFLFPPKPKDTLMIS
RTPEVTCVVVDVSHEDPEVKFNWYVDGVEVHNAKTKPREEQYNSTYRVVSVLTVLA
(*)QDWLNGKEYKCKVSNKALPAPIEKTISKA(*)KGQPREPQVYTLPPSREEMTKNQV
SLTCLVKGFYPSDIAVEWESNGQPENNYKTTPPVLDSDGSFFLYSKLTVDKSRWQQG
NVFSCSVMHEALHNAYTQKSLSLSPGGGGGSEQKLISEEDL
SEQ ID NO: 26: Human Fc-IgG1 with the addition of ISAMVRS amino acid sequence
(underlined) at the N-terminus, alterations of the H310A(*) and H435A(*) to prevent FcRn
binding, and additions of a G4S connector (italic) and a c-Myc tag (underlined, italic) at
the C-terminus
ISAMVRSDKTHTCPPCPAPELLGGPSVFLFPPKPKDTLMISRTPEVTCVVVDVSHEDPE
VKFNWYVDGVEVHNAKTKPREEQYNSTYRVVSVLTVLA(*)QDWLNGKEYKCKVSN
KALPAPIEKTISKA(*)KGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNAYTQ
KSLSLSPGGGGGSEQKLISEEDL
SEQ ID NO: 27: Human Fc-IgG1 with additions of the human serum albumin signal
sequence (bold) and ISAMVRS amino acid sequence (underlined) at the N-terminus and
additions of a G4S connector (italic), a mutation (lysine to phenylalanine, bold) and a
c-Myc tag (underlined, italic) at the C-terminus
MKWVTFISLLFLFSSAYSISAMVRSDKTHTCPPCPAPELLGGPSVFLFPPKPKDTLMIS
RTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFS
CSVMHEALHNHYTQKSLSLSPGGGGGSEQFLISEEDL
SEQ ID NO: 28: Mature human Fc-IgG1 with addition of the ISAMVRS amino acid sequence
at the N-terminus (underlined) and additions of a G4S connector (italic), a mutation (lysine
to phenylalanine, bold) and a c-Myc tag (underlined, italic) at the C-terminus
ISAMVRSDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSL
SLSPGGGGGSEQFLISEEDL
SEQ ID NO: 29: Human Fc-IgG1 with addition of human serum albumin signal sequence (bold)
and ISAMVRS amino acid sequence (underlined) at the N-terminus, substitution (*) from Asn
to Ala and additions of a G4S connector (italic), a mutation (lysine to phenylalanine, bold)
and a c-Myc tag (underlined, italic) at the C-terminus
MKWVTFISLLFLFSSAYSISAMVRSDKTHTCPPCPAPELLGGPSVFLFPPKPKDTLMIS
RTPEVTCVVVDVSHEDPEVKFNWYVDGVEVHNAKTKPREEQYA(*)STYRVVSVLTV
LHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNV
FSCSVMHEALHNHYTQKSLSLSPGGGGGSEQFLISEEDL
SEQ ID NO: 30: Mature human Fc-IgG1 with addition of the ISAMVRS amino acid sequence
(underlined) at the N-terminus, substitution (*) from Asn to Ala and additions of a G4S
connector (italic), a mutation (lysine to phenylalanine, bold) and a c-Myc tag (underlined,
italic) at the C-terminus
ISAMVRSDKTHTCPPCPAPELLGGPSVFLFPPKPKDTLMISRTPEVTCVVVDVSHEDPE
VKFNWYVDGVEVHNAKTKPREEQYA(*)STYRVVSVLTVLHQDWLNGKEYKCKVSN
KALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKS
LSLSPGGGGGSEQFLISEEDL
SEQ ID NO: 31: Human Fc-IgG1 with addition of human serum albumin signal sequence
(bold) at the N-terminus (bold), possession of the allotype Glm(fa) (bold italic), and
additions of a G4S connector (italic), a mutation (lysine to phenylalanine, bold) and a
c-Myc tag (underlined, italic) at the C-terminus
MKWVTFISLLFLFSSAYSDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHE
ALHNHYTQKSLSLSPGGGGGSEQFLISEEDL
SEQ ID NO: 32: Human Fc-IgG1 with addition of human serum albumin signal sequence (bold)
at the N-terminus and possession of the allotype G1m(fa) (bold italic)
MKWVTFISLLFLFSSAYSDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHE
ALHNHYTQKSLSLSPGK
SEQ ID NO: 33: Mature human Fc-IgG1 with addition of the MVRS amino acid sequence
(underlined) at the N-terminus and possession of the YTE triple mutation (bold, underlined)
MVRSDKTHTCPPCPAPELLGGPSVFLFPPKPKDTLYITREPEVTCVVVDVSHEDPEVK
FNWYVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLS
PGK
SEQ ID NO: 34: Human Fc-IgG1 with additions of a human serum albumin signal sequence
(bold) and the EPKSS amino acid sequence (underlined) of the hinge region of mature human
Fc-IgG1 at the N-terminus, and possessions of cysteine-to-serine alteration (#) and the
allotype G1m(fa) (bold italic)
MKWVTFISLLFLFSSAYSEPKSS(#)DKTHTCPPCPAPELLGGPSVFLFPPKPKDTLMIS
RTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFS
CSVMHEALHNHYTQKSLSLSPGK
SEQ ID NO: 35: Human Fc-IgG1 with, at the N-terminus, addition of a murine IgG signal
sequence (bold) and removal of the EPKSSD amino acid sequence from the hinge region of
mature human Fc-IgG, and possession of the allotype G1m(fa) (bold italic)
MGWSCIILFLVATATGVHSKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHE
ALHNHYTQKSLSLSPGK
SEQ ID NO: 36: Mature human Fc-IgG1 with removal of the EPKSSD amino acid sequence
from the hinge region of mature human Fc-IgG, at the N-terminus, and possessions of the
allotype G1m(fa) (bold italic) and the YTE triple mutation (bold, underlined)
KTHTCPPCPAPELLGGPSVFLFPPKPKDTLYITREPEVTCVVVDVSHEDPEVKFNWYV
DGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 37: Mature human Fc-IgG1 with removal of the EPKSSD amino acid sequence
from the hinge region of mature human Fc-IgG, at the N-terminus, and possessions of the LS
double mutation (bold, underlined) and the allotype G1m(fa) (bold italic)
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKT
TPPVLDSDGSFFLYSKLTVDKSRWQQGNVFSCSVLHEALHSHYTQKSLSLSPGK
SEQ ID NO: 38: Human Fc-IgG1 with addition of human serum albumin signaling sequence
(bold) at the N-terminus, possessions of the YTE triple mutation (bold, underlined) and the
allotype G1m(fa) (bold italic), and additions of a G4S connector (italic) and a c-Myc tag
(underlined) at the C-terminus
MKWVTFISLLFLFSSAYSDKTHTCPPCPAPELLGGPSVFLFPPKPKDTLYITREPEVTC
VVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHE
ALHNHYTQKSLSLSPGGGGGSEQKLISEEDL
SEQ ID NO: 39: Mature human Fc-IgG1, wherein X₁ is Met or Trp, X₂ is Ser or Thr, X₃ is Thr
or Glu, X₄ is Asp or Glu, and X₅ is Leu or Met, X₆ is Met or Leu, and X₇ is Asn or Ser
DKTHTCPPCPAPELLGGPSVFLFPPKPKDTLX₁IX₂RX₃PEVTCVVVDVSHEDPEVKEN
WYVDGVEVHNAKTKPREEQYNSTYRVVSVLTVLHQDWLNGKEYKCKVSNKALPAP
IEKTISKAKGQPREPQVYTLPPSRX₄EX₅TKNQVSLTCLVKGFYPSDIAVEWESNGQPE
NNYKTTPPVLDSDGSFFLYSKLTVDKSRWQQGNVFSCSVX₆HEALHX₇HYTQKSLSLS
PG
SEQ ID NO: 40: Mature human Fc-IgG1, wherein X₄ is Asp or Glu, and X₅ is Leu or Met
DKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEK
TISKAKGQPREPQVYTLPPSRX₄EX₅TKNQVSLTCLVKGFYPSDIAVEWESNGQPENNY
KTTPPVLDSDGSFFLYSKLTVDKSRWQQGNVFSCSVMHEALHNHYTQKSLSLSPG
SEQ ID NO: 41: Mature human Fc-IgG1 with the YTE triple mutation (bold, underlined),
wherein X₄ is Asp or Glu, and X₅ is Leu or Met
DKTHTCPPCPAPELLGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALPAPIEK
TISKAKGQPREPQVYTLPPSRX₄EX₅TKNQVSLTCLVKGFYPSDIAVEWESNGQPENNY
KTTPPVLDSDGSFFLYSKLTVDKSRWQQGNVFSCSVMHEALHNHYTQKSLSLSPG
SEQ ID NO: 42: Mature human Fc-IgG1 with the YTE triple mutation (bold, underlined) and
the allotype G1m(fa) (Bold Italic)
DKTHTCPPCPAPELLGGPSVFLFPPKPKDTLYITREPEVTCVVVDVSHEDPEVKENWY
VDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPG
SEQ ID NO: 43: Mature human Fc-IgGI with the YTE triple mutation (bold, underlined) and
the allotype G1m(f) (Bold Italic)
DKTHTCPPCPAPELLGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPG
SEQ ID NO: 44: Mature human Fc-IgG1 with the LS double mutation (bold, underlined),
wherein X₄ is Asp or Glu, and X₅ is Leu or Met
DKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEK
TISKAKGQPREPQVYTLPPSRX₄EX₅TKNQVSLTCLVKGFYPSDIAVEWESNGQPENNY
KTTPPVLDSDGSFFLYSKLTVDKSRWQQGNVFSCSVLHEALHSHYTQKSLSLSPG
SEQ ID NO: 45: Mature human Fc-IgG1 with the LS double mutation (bold, underlined) and
allotype G1m(fa) (Bold Italic)
DKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYK
TTPPVLDSDGSFFLYSKLTVDKSRWQQGNVFSCSVLHEALHSHYTQKSLSLSPG
SEQ ID NO: 46: Mature human Fc-IgG1 with the LS double mutation (bold, underlined) and
allotype G1m(f) (Bold Italic)
DKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNY
KTTPPVLDSDGSFFLYSKLTVDKSRWQQGNVFSCSVLHEALHSHYTQKSLSLSPG
SEQ ID NO: 47: Mature human Fc-IgG1 with addition of mouse heavy chain MIgG Vh
signaling sequence (bold) at the N-terminus and alteration (#) from cysteine to serine,
wherein X₁ is Met or Trp, X₂ is Ser or Thr, X₃ is Thr or Glu, X₄ is Asp or Glu, and X₅ is Leu
or Met, X₆ is Met or Leu, and X₇ is Asn or Ser
MGWSCIILFLVATATGVHSNVNHKPSNTKVDKKVEPKSS(#)DKTHTCPPCPAPELLG
GPSVFLFPPKPKDTLX₁IX₂RX₃PEVTCVVVDVSHEDPEVKFNWYVDGVEVHNAKTKP
REEQYNSTYRVVSVLTVLHQDWLNGKEYKCKVSNKALPAPIEKTISKAKGQPREPQV
YTLPPSRX₄EX₅TKNQVSLTCLVKGFYPSDIAVEWESNGQPENNYKTTPPVLDSDGSFF
LYSKLTVDKSRWQQGNVFSCSVX₆HEALHX₇HYTQKSLSLSPG
SEQ ID NO: 48: Mature human Fc-IgGI with addition of mouse heavy chain MIgG Vh
signaling sequence (bold) at the N-terminus, alteration (#) from cysteine to serine and
possession of allotype G1m(fa) (Bold Italic)
MGWSCIILFLVATATGVHSNVNHKPSNTKVDKKVEPKSS(#)DKTHTCPPCPAPELLG
GPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 49: Mature human Fc-IgG1 with addition of mouse heavy chain MIgG Vh
signaling sequence (bold) at the N-terminus, alteration (#) from cysteine to serine and
possession of allotype G1m(f) (Bold Italic)
MGWSCIILFLVATATGVHSNVNHKPSNTKVDKKVEPKSS(#)DKTHTCPPCPAPELLG
GPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 50: Mature human Fc-IgG1 with addition of mouse heavy chain MIgG Vh
signaling sequence (bold) at the N-terminus, alteration (#) from cysteine to serine, and
possessions of M428L and N434S mutations and the allotype G1m(fa) (Bold Italic)
MGWSCIILFLVATATGVHSNVNHKPSNTKVDKKVEPKSS(#)DKTHTCPPCPAPELLG
GPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYS
KLTVDKSRWQQGNVFSCSVLHEALHSHYTQKSLSLSPGK
SEQ ID NO: 51: Mature human Fc-IgG1 with addition of mouse heavy chain MIgG Vh
signaling sequence (bold) at the N-terminus, alteration (#) from cysteine to serine, and
possessions of M428L and N434S mutations and the allotype G1m(f) (Bold Italic)
MGWSCIILFLVATATGVHSNVNHKPSNTKVDKKVEPKSS(#)DKTHTCPPCPAPELLG
GPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDSDGSFFLYS
KLTVDKSRWQQGNVFSCSVLHEALHSHYTQKSLSLSPGK
SEQ ID NO: 52: Mature human Fc-IgG1 with addition of mouse heavy chain MIgG Vh
signaling sequence (bold) at the N-terminus, alteration (#) from cysteine to serine, and
possessions of YTE triple mutations (bold, underlined) and the allotype Glm(fa) (bold italic)
MGWSCIILFLVATATGVHSNVNHKPSNTKVDKKVEPKSS(#)DKTHTCPPCPAPELLG
GPSVFLFPPKPKDTLYITREPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 53: Mature human Fc-IgG1 with addition of mouse heavy chain MIgG Vh
signaling sequence (bold) at the N-terminus, alteration (#) from cysteine to serine, and
possessions of YTE triple mutations (bold, underlined) and the allotype G1m(f) (bold italic)
MGWSCIILFLVATATGVHSNVNHKPSNTKVDKKVEPKSS(#)DKTHTCPPCPAPELLG
GPSVFLFPPKPKDTLYITREPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 54: Mature human Fc-IgG1 with additions of mouse heavy chain MIgG Vh
signaling sequence (bold) and the ISAMVRS amino acid sequence (italic) at the N-terminus,
mutations of M428L and N434S (bold, underlined), additions of a G4S connector (italic) and a
c-Myc tag (underlined) at the C-terminus, and possession of the allotype G1m(f) (bold italic)
MGWSCIILFLVATATGVHSISAMVRSDKTHTCPPCPAPELLGGPSVFLFPPKPKDTLMI
SRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVF
SCSVLHEALHSHYTQKSLSLSPGGGGGSEQKLISEEDL
SEQ ID NO: 55: Mature human Fc-IgG1 with additions of mouse heavy chain MIgG Vh
signaling sequence (bold) and the ISAMVRS amino acid sequence (italic) at the N-terminus,
mutations of M428L and N434S (bold, underlined), additions of a G4S connector (italic) and a
c-Myc tag (underlined) at the C-terminus, and possession of the allotype Glm(fa) (bold italic)
MGWSCIILFLVATATGVHSISAMVRSDKTHTCPPCPAPELLGGPSVFLFPPKPKDTLMI
SRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVF
SCSVLHEALHSHYTQKSLSLSPGGGGGSEQKLISEEDL
SEQ ID NO: 56: Mature human Fc-IgG1 with additions of mouse heavy chain MIgG Vh
signaling sequence (bold) and the ISAMVRS amino acid sequence (italic) at the N-terminus,
triple mutations of YTE (bold, underlined), additions of a G4S connector (italic) and a c-Myc
tag (underlined) at the C-terminus, and possession of the allotype G1m(f) (bold italic)
MGWSCIILFLVATATGVHSISAMVRSDKTHTCPPCPAPELLGGPSVFLFPPKPKDTLYI
TREPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNV
FSCSVMHEALHNHYTQKSLSLSPGGGGGSEQKLISEEDL
SEQ ID NO: 57: Mature human Fc-IgG1 with additions of mouse heavy chain MIgG Vh
signaling sequence (bold) and the ISAMVRS amino acid sequence (italic) at the N-terminus,
triple mutations of YTE (bold, underlined), additions of a G4S connector (italic) and a c-Myc
tag (underlined) at the C-terminus, and possession of the allotype Glm(fa) (bold italic)
MGWSCIILFLVATATGVHSISAMVRSDKTHTCPPCPAPELLGGPSVFLFPPKPKDTLYI
TREPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNV
FSCSVMHEALHNHYTQKSLSLSPGGGGGSEQKLISEEDL
SEQ ID NO: 58: Mature human Fc-IgG1 with addition of mouse heavy chain MIgG Vh
signaling sequence (bold) at the N-terminus, alteration (#) from cysteine to serine,
additions o fa G4S connector (italic) and an IgA peptide tag (underlined) at the C-terminus,
and possession of the allotype G1m(fa) (bold italic)
MGWSCIILFLVATATGVHSEPKSS(#)DKTHTCPPCPAPELLGGPSVFLFPPKPKDTLMI
SRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVF
SCSVMHEALHNHYTQKSLSLSPGGGGGSQRNPRLRLIRRHPTLRIPPI
SEQ ID NO: 59: Mature human Fc-IgG1 with addition of mouse heavy chain MIgG Vh signaling
sequence (bold) at the N-terminus, alteration (#) from cysteine to serine, mutations of
M428L and N434S (bold, underlined), additions of a G4S connector (italic) and an IgA peptide
tag (underlined) at the C-terminus, and possession of the allotype G1m(fa) (bold italic)
MGWSCIILFLVATATGVHSEPKSS(#)DKTHTCPPCPAPELLGGPSVFLFPPKPKDTLMI
SRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVF
SCSVLHEALHSHYTQKSLSLSPGGGGGSQRNPRLRLIRRHPTLRIPPI
SEQ ID NO: 60: Mature human Fc-IgG1, wherein Z1 is Cys or Ser, X₁ is Met or Trp, X₂ is Ser
or Thr, X₃ is Thr or Glu, X₄ is Asp or Glu, and X5 is Leu or Met, X₆ is Met or Leu, and X₇
is Asn or Ser
NVNHKPSNTKVDKKVEPKSZ₁DKTHTCPPCPAPELLGGPSVFLFPPKPKDTLX₁IX₂RX₃
PEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRX₄EX₅TKNQVSLTC
LVKGFYPSDIAVEWESNGQPENNYKTTPPVLDSDGSFFLYSKLTVDKSRWQQGNVFS
CSVX₆HEALHX₇HYTQKSLSLSPGK
SEQ ID NO: 61: Mature human Fc-IgG1 with a cysteine-to-serine alteration (#), wherein X₁ is
Met or Trp, X₂ is Ser or Thr, X₃ is Thr or Glu, X₄ is Asp or Glu, and X₅ is Leu or Met, X₆
is Met or Leu, and X₇ is Asn or Ser
NVNHKPSNTKVDKKVEPKSS(#)DKTHTCPPCPAPELLGGPSVFLFPPKPKDTLX₁IX₂R
X₃PEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRX₄EX₅TKNQVSLT
CLVKGFYPSDIAVEWESNGQPENNYKTTPPVLDSDGSFFLYSKLTVDKSRWQQGNVF
SCSVX₆HEALHX₇HYTQKSLSLSPGK
SEQ ID NO: 62: Mature human Fc-IgG1 with a cysteine-to-serine alteration (#), wherein X₄ is
Asp or Glu, and X₅ is Leu or Met
NVNHKPSNTKVDKKVEPKSS(#)DKTHTCPPCPAPELLGGPSVFLFPPKPKDTLMISRTP
EVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRX₄EX₅TKNQVSLTCL
VKGFYPSDIAVEWESNGQPENNYKTTPPVLDSDGSFFLYSKLTVDKSRWQQGNVFSC
SVMHEALHNHYTQKSLSLSPGK
SEQ ID NO: 63: Mature human Fc-IgG1 with a cysteine-to-serine alteration (#) and the
allotype G1m(f) (bold italic)
NVNHKPSNTKVDKKVEPKSS(#)DKTHTCPPCPAPELLGGPSVFLFPPKPKDTLMISRTP
EVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCS
VMHEALHNHYTQKSLSLSPGK
SEQ ID NO: 64: Mature human Fc-IgG1 with a cysteine-to-serine alteration (#) and the
allotype G1m(fa) (bold italic)
NVNHKPSNTKVDKKVEPKSS(#)DKTHTCPPCPAPELLGGPSVFLFPPKPKDTLMISRTP
EVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCS
VMHEALHNHYTQKSLSLSPGK
SEQ ID NO: 65: Mature human Fc-IgG1 with a cysteine-to-serine alteration (#), M428L and
N343S mutations(bold, underlined), and the allotype Glm(fa) (bold italic)
NVNHKPSNTKVDKKVEPKSS(#)DKTHTCPPCPAPELLGGPSVFLFPPKPKDTLMISRTP
EVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCS
VLHEALHSHYTQKSLSLSPGK
SEQ ID NO: 66: Mature human Fc-IgG1 with a cysteine-to-serine alteration (#), M428L and
N343S mutations(bold, underlined), and the allotype G1m(f) (bold italic)
NVNHKPSNTKVDKKVEPKSS(#)DKTHTCPPCPAPELLGGPSVFLFPPKPKDTLMISRTP
EVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCS
VLHEALHSHYTQKSLSLSPGK
SEQ ID NO: 67: Mature human Fc-IgG1 with a cysteine-to-serine alteration (#), YTE triple
mutations(bold, underlined), and the allotype G1m(fa) (bold italic)
NVNHKPSNTKVDKKVEPKSS(#)DKTHTCPPCPAPELLGGPSVFLFPPKPKDTLYITRE
PEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSC
SVMHEALHNHYTQKSLSLSPGK
SEQ ID NO: 68: Mature human Fc-IgGI with a cysteine-to-serine alteration (#), YTE triple
mutations(bold, underlined), and the allotype G1m(f) (bold italic)
NVNHKPSNTKVDKKVEPKSS(#)DKTHTCPPCPAPELLGGPSVFLFPPKPKDTLYITRE
PEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTKNQVSLTCL
VKGFYPSDIAVEWESNGQPENNYKTTPPVLDSDGSFFLYSKLTVDKSRWQQGNVFSC
SVMHEALHNHYTQKSLSLSPGK

### BRIEF DESCRIPTION OF THE DRAWINGS

FIG. 1: SEC-HPLC of conjugate 1.

FIG. 2: SEC-HPLC of conjugate 2.

FIG. 3: SEC-HPLC of conjugate 3.

FIG. 4: SEC-HPLC of conjugate 4.

FIG. 5: SEC-HPLC of conjugate 5.

FIG. 6: SEC-HPLC of conjugate 6.

FIG. 7: SEC-HPLC of conjugate 7.

FIG. 8: SEC-HPLC of conjugate 8.

FIG. 9: SEC-HPLC of conjugate 9.

FIG. 10: LCMS of conjugate 1.

FIG. 11: LCMS of conjugate 2.

FIG. 12: LCMS of conjugate 3.

FIG. 13: LCMS of conjugate 4.

FIG. 14: LCMS of conjugate 5.

FIG. 15: LCMS of conjugate 6.

FIG. 16: LCMS of conjugate 7.

FIG. 17: LCMS of conjugate 8.

FIG. 18: LCMS of conjugate 9.

FIG. 19: Residual free drug content of conjugate 1.

FIG. 20: Residual free drug content of conjugate 2.

FIG. 21: Residual free drug content of conjugate 3.

FIG. 22: Residual free drug content of conjugate 4.

FIG. 23: Residual free drug content of conjugate 5.

FIG. 24: Residual free drug content of conjugate 6.

FIG. 25: Residual free drug content of conjugate 7.

FIG. 26: Residual free drug content of conjugate 8.

FIG. 27: Residual free drug content of conjugate 9.

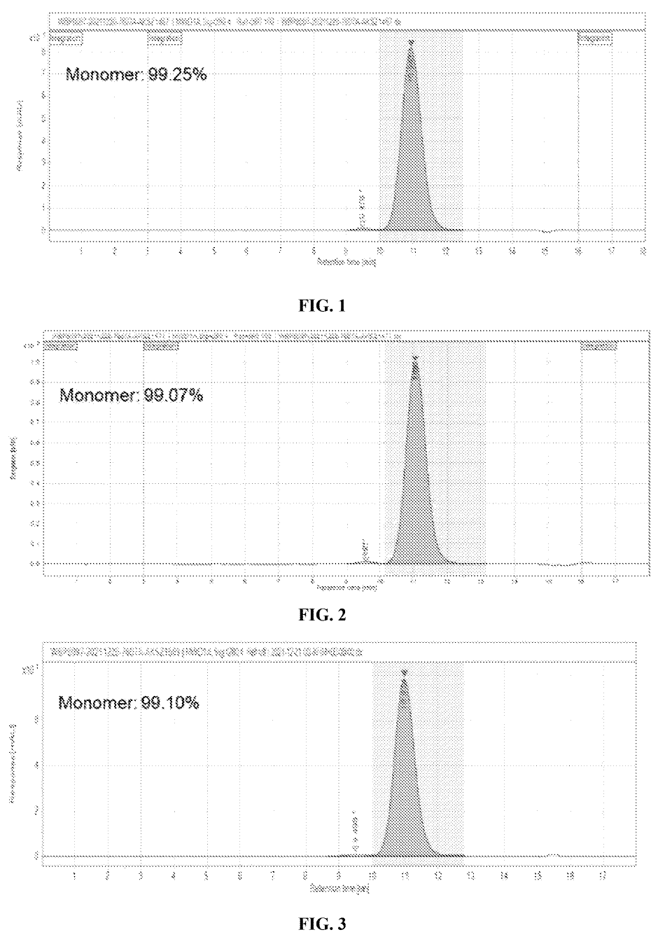

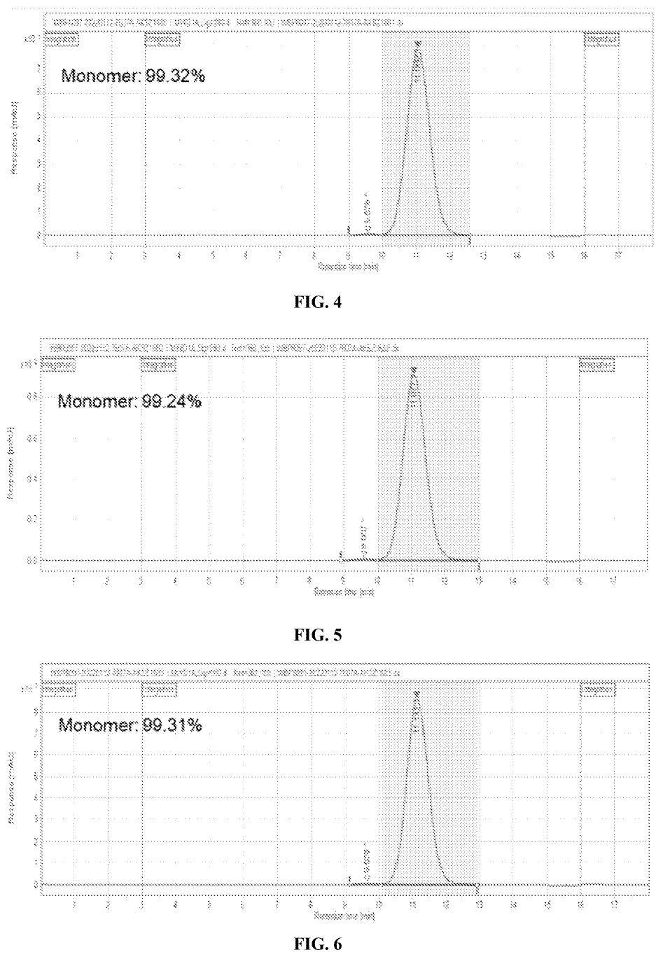

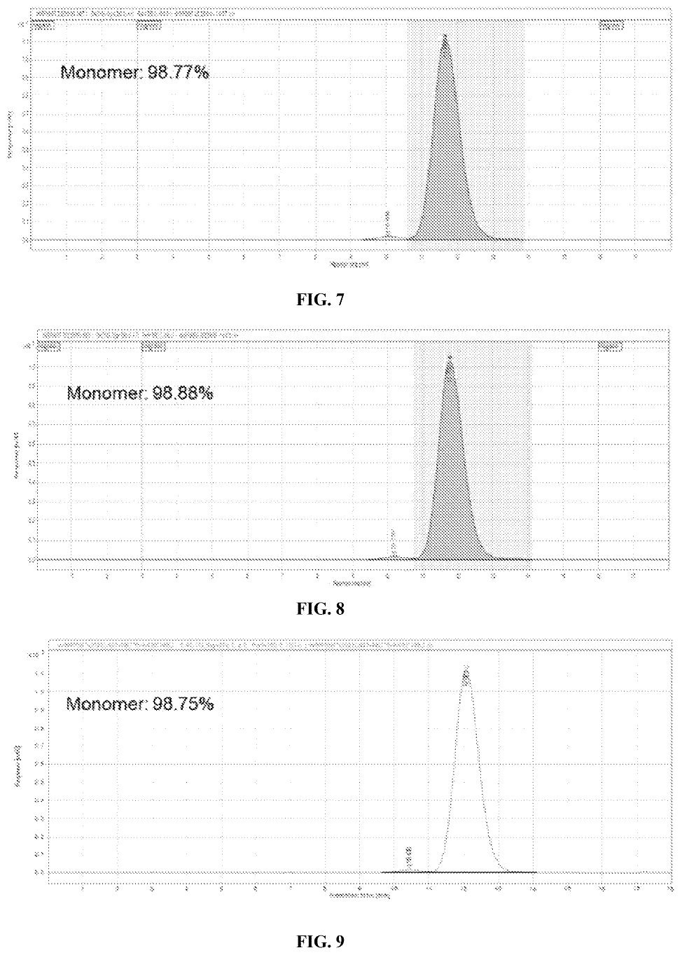

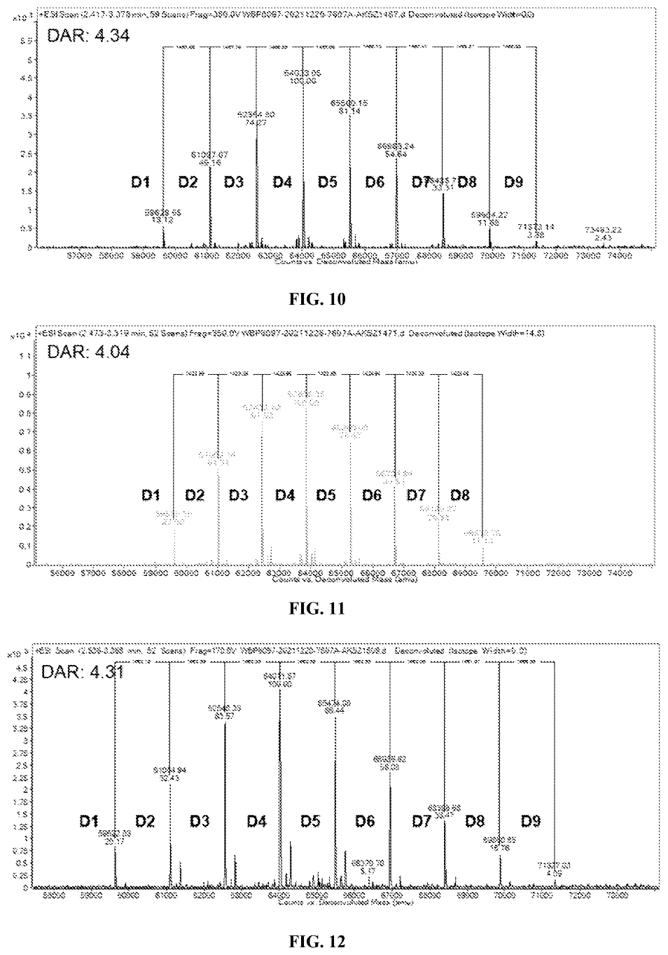

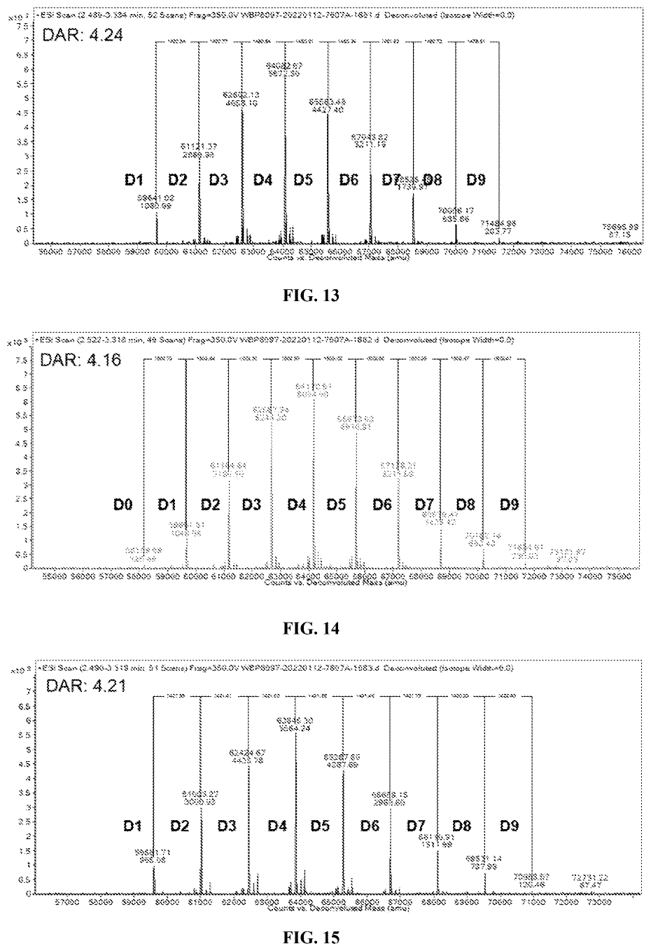

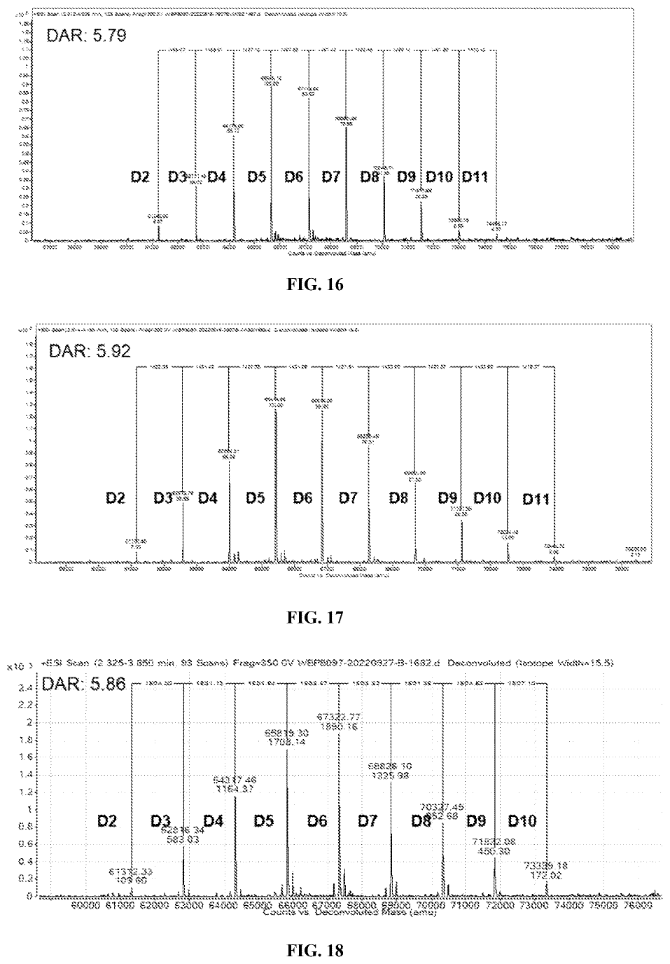

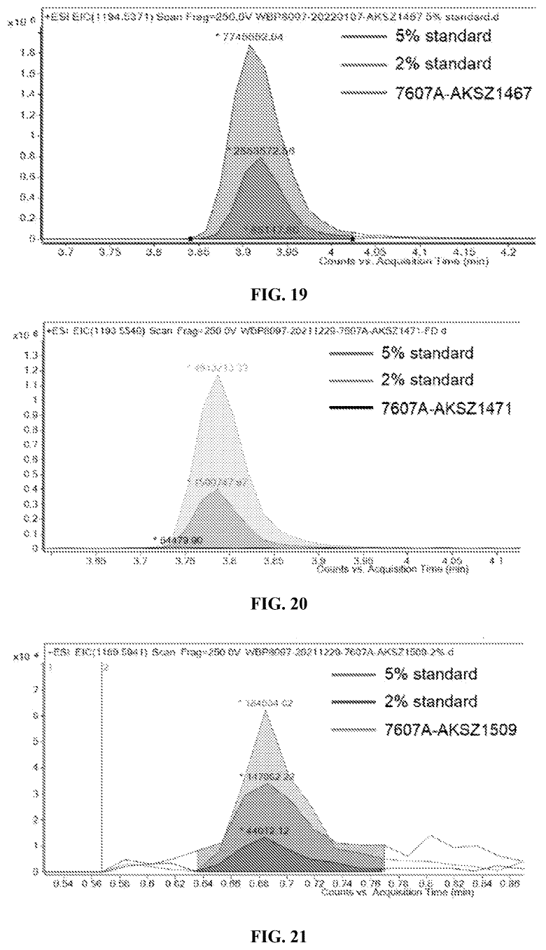

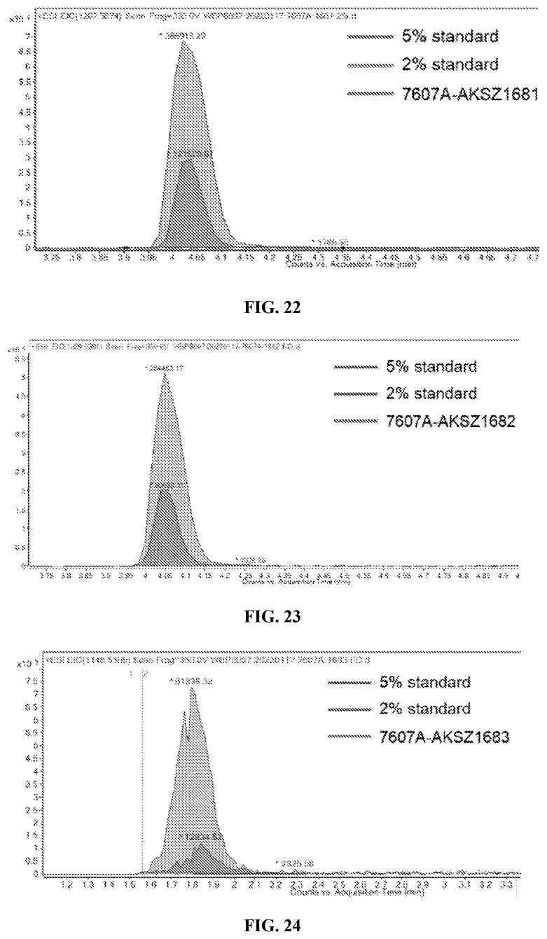

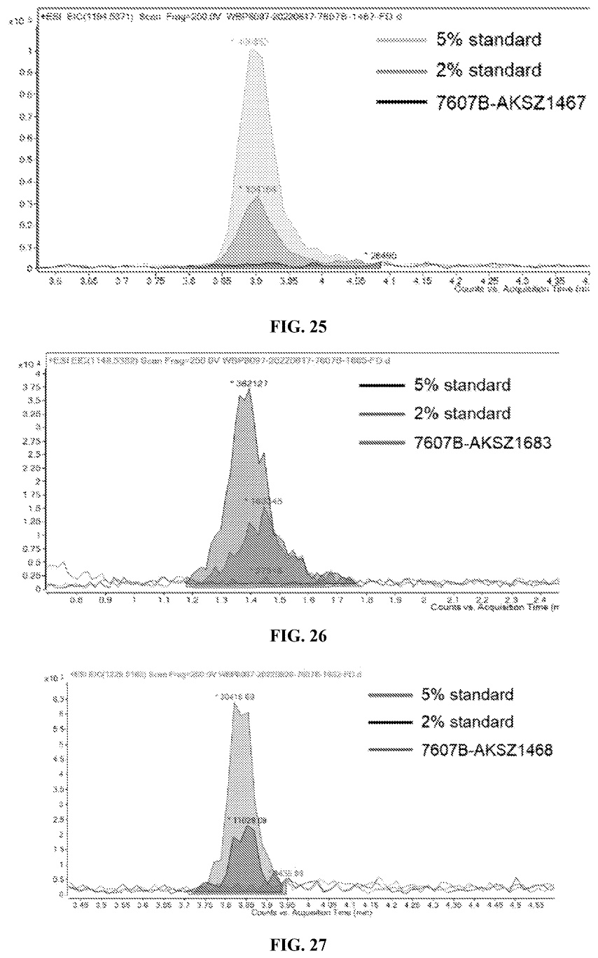

### DETAILED DESCRIPTION

These and other aspects and embodiments of the present disclosure are exemplified in the following embodiments. Any or all of the features discussed above and throughout this application may be combined in various embodiments of the present disclosure. The following embodiments further illustrate the present disclosure, however, it should be understood that the embodiments are described in an illustrative and not limiting manner and that a variety of modifications may be made by the skilled in the art.

### Example 1: Preparation of Fc Portion

The amino acid sequences of the protein (SEQ ID Nos:1-68) were reverse-translated to synthesize corresponding nucleotide sequences. The nucleotide sequence with appropriate cleavage sites (XbaI+SalI) at its two ends was cloned into the pWX4.1 expression vector (WuXi Biotechnology Co., Ltd., Shanghai, China). Each constructed vector carries either the signal peptide sequence of human interleukin 2 or the signal peptide sequence of human serum albumin. The pWX4.1 plasmid was transfected into E. coli Top10 strain (Life Technologies) for DNA amplification and purified using the PURELINK® HiPURE Plasmid Filter Maxiprep Kit (Life Technologies). Then, the plasmids were then transfected into CHO cells (ATCC) using the EXPIFECTAMINE™ 293 Transfection Kit (Life Technologies). The transfected cells were cultured for 7 days, then centrifuged and precipitated, and filtered. The supernatant was collected and purified using MabSelect Sure Resin (GE Healthcare, Chicago, IL, USA) to obtain purified h-IgG Fc. The purified samples were subjected to 4-12% Bis Tris SDS-PAGE electrophoresis and 1-2 g of the sample were used, and then stained with Thomas Brilliant Blue. Each sample was subjected to both reducing (R) and non-reducing (NR) treatments.

### Example 2: General Synthesis Method of h-IgG1 Fc-PEG4-azides

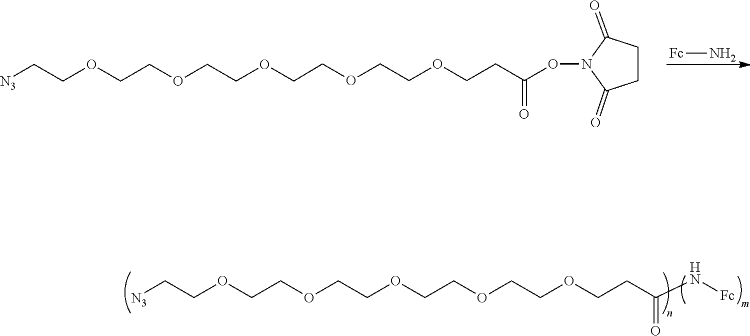

At 0° C., PEG-azide-NHS ester (1 to 20 equivalents) was dissolved in a solution of DMF/PBS, which was then added to a PBS solution of h-IgG1 Fc prepared in Example 1. The reaction was stirred at room temperature for 1 to 10 h. The reaction system was concentrated by centrifugation, and the PBS solution was used for washing. The target h-IgG1 Fc-PEG4-azide was prepared by preparative chromatography.

### Example 3: Generalized Synthesis Method of Conjugates

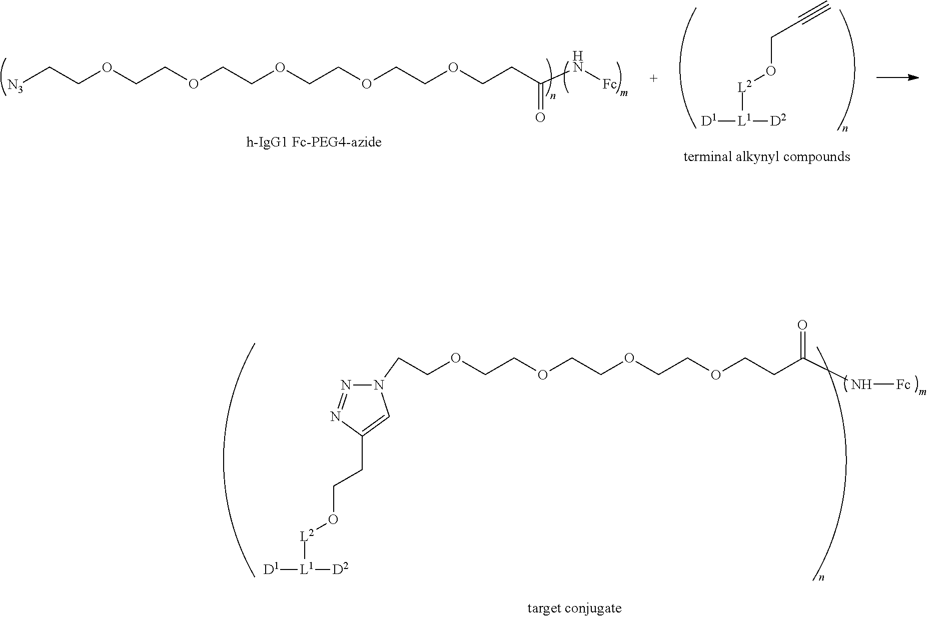

The h-IgG1 Fc-PEG4-azide dissolved in PBS solution was added to the PBS solution of terminal alkynyl compounds, copper sulfate, tris(3-hydroxypropyltriazolylmethyl)-amine, and sodium ascorbate. The reaction solution was reacted for at least 12 hours, then diluted with PBS, and concentrated by centrifugation. Then, the PBS solution was used for washing, and the target conjugate was prepared by preparative chromatography. The average DAR (drug/antibody ratio) of the conjugate was analyzed by MS.

### Example 4: Synthesis of Intermediate Drug (INT-DRUG)

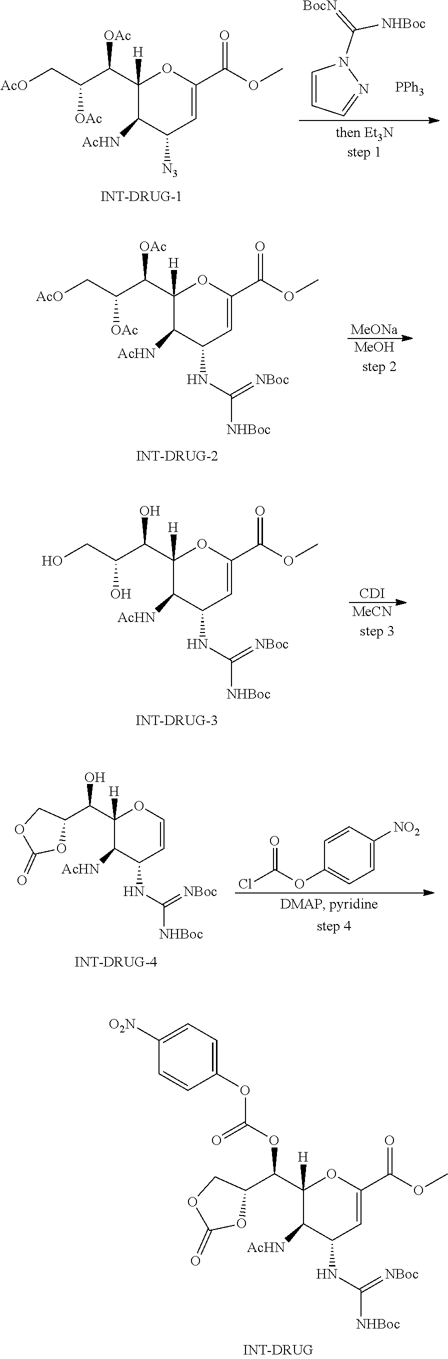

### Step One: Synthesis of INT-DRUG-2

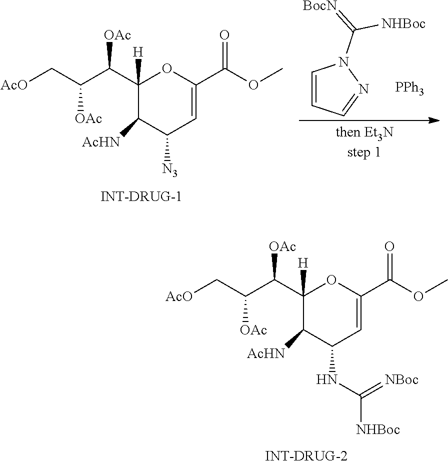

INT-DRUG-1 (3.0 g, 6.57 mmol) was added to the reaction flask, followed by the additions of the tetrahydrofuran (30 mL), triphenylphosphine (1.9 g, 7.23 mmol), and the reaction was stirred at 30° C. for 2 h. after this reaction ends, lithium hydroxide monohydrate (28 mg) and water (1.5 mL) were added, and the reaction continues to stir at 30° C. for 16 h. Once TLC monitors the end of the reaction, N,N′-di-Boc-1H-1-guanidinium pyrazole (2.1 g, 6.91 mmol) was added and stirred at 30° C. for 48 h. After the reaction finishes, the reaction solution was concentrated, and separated and purified by column chromatography on silica gel (ethyl acetate/petroleum ether-1:1). The colorless oil INT-DRUG-2 (2.2 g) was obtained after purification. LCMS: [M+1]⁺ found 673.40

### Step Two: Synthesis of INT-DRUG-3

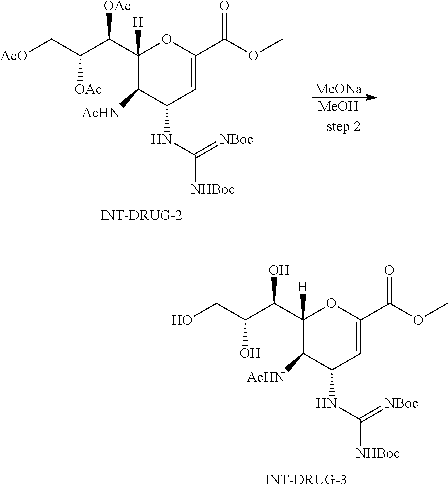

Under nitrogen protection, INT-DRUG-2 (2.19 g, 3.25 mmol) was added to a reaction flask, and then a methanol (8 mL) solution of dried sodium methanolate (0.65 mmol). The reaction solution was stirred at room temperature for 10 min, and quenched with HCl (0.4N, 1,4-dioxane solution, 2.5 mL). The reaction solution was concentrated, separated and purified by silica gel column chromatography (ethyl acetate/petroleum ether, 70%˜100%) to give a white solid INT-DRUG-3 (1.15 g). LCMS: [M+1]+ found 547.33.

### Step Three: Synthesis of INT-DRUG-4

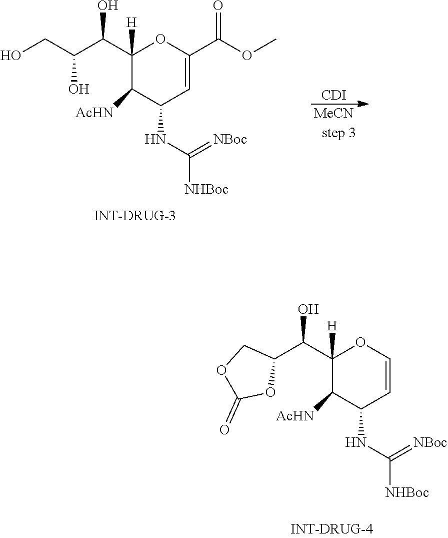

INT-DRUG-3 (386.9 mg, 0.71 mmol), acetonitrile (10 mL), CDI (160.8 mg, 0.99 mmol), and DMAP (302.7 mg, 2.47 mmol) were added to the reaction flask. The reaction was stirred at room temperature for 16 hours. At the end of the reaction, the reaction solution was concentrated and separated and purified by silica gel column chromatography (ethyl acetate/petroleum ether, 0%˜45%) to obtain a white solid INT-DRUG-4 (242.8 mg). LCMS: [M+1]+ found 573.35.

### Step Four: Synthesis of INT-DRUG-5

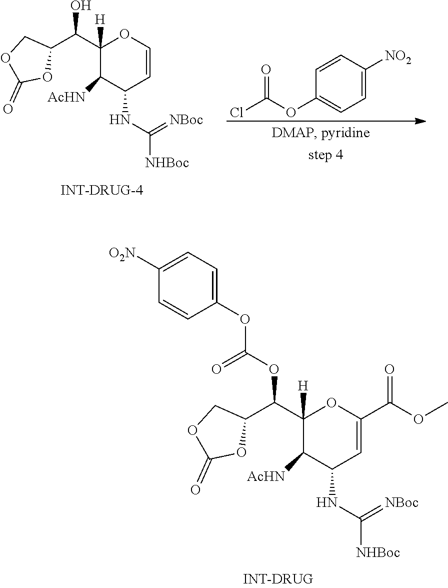

INT-DRUG-4 (403 mg, 0.7 mmol), DMAP (1.37 g, 11.2 mmol), pyridine (60 mL), and phenyl p-nitrochloroformate (2.11 g, 10.5 mmol) were added to the reaction flask and stirred at 40° C. for 4 h after nitrogen displacement. The reaction was quenched (can not be left for a long time) with water (20 mL) when the rection conversion detected by LCMS was to be >90%. The reverse column was used for separation (acetonitrile/water 0%˜90%), and the white solid INT-DRUG (173 mg) was obtained by freeze-drying. LCMS: [M+1]+ found 738.33.

### Example 5: Synthesis of Intermediate a (Inter-A)

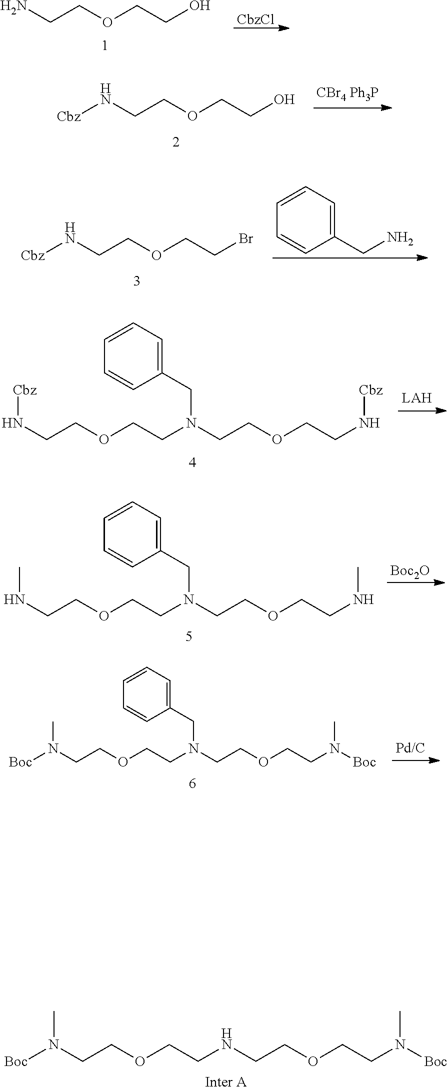

### Step One: Synthesis of Intermediate 2

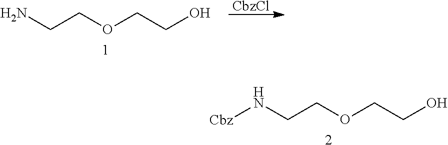

Compound 1 (150 g, 1.43 mol) and triethylamine (289 g, 2.85 mol) were added to the reaction flask followed by addition of tetrahydrofuran (1 L), which was then stirred at 0° C. after nitrogen displacement. The CbzCl (243 g, 1.43 mol) dissolved in tetrahydrofuran (500 mL) was added dropwise to the reaction solution. The reaction solution was stirred at room temperature overnight. At the end of the reaction, the reaction was quenched by the addition of water (3 L) and extracted with ethyl acetate (300 mL×2). The combined organic phases were washed with saturated saline, dried with anhydrous sodium sulfate and concentrated to obtain the crude product. The crude product was purified by silica gel column chromatography (dichloromethane/methanol, 20/1) to give the yellow oil compound 2 (255 g).

### Step Two: Synthesis of Intermediate 3

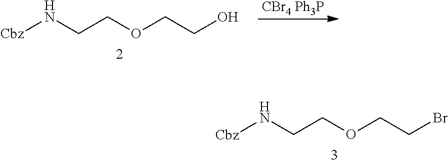

Compound 2 (245 g, 1.02 mol) and carbon tetrabromide (509 g, 1.54 mol) were added to the reaction flask followed by addition of the dichloromethane (2 L), which was then stirred at 0° C. after nitrogen displacement. Triphenylphosphine (403 g, 1.54 mol) dissolved in dichloromethane (500 mL) was added dropwise to the reaction solution. The reaction solution was stirred at room temperature for 1.5 h and concentrated to obtain the crude product at the end of the reaction. The crude product was purified by silica gel column chromatography (dichloromethane/methanol, 20/1) to give the yellow oil compound 3 (200 g, crude).

### Step Three: Synthesis of Intermediate 4

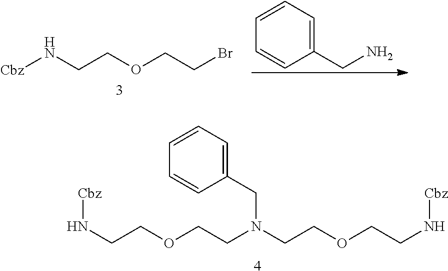

Compound 3 (200 g, crude) and N,N-dimethylformamide (2 L) was added to the reaction flask. Byramine (28.3 g, 264 mmol) and sodium carbonate (73.0 g, 528 mmol) were added to the reaction solution. The reaction solution was stirred at 60° C. overnight. At the end of the reaction, the temperature was reduced to room temperature and the reaction solution was added with water (3 L) and extracted with ethyl acetate (300 mL×2). The combined organic phases were washed with saturated saline, then dried with anhydrous sodium sulfate and concentrated to obtain the crude product. The crude product was purified by silica gel column chromatography (dichloromethane/methanol, 20/1) to give the yellow oil compound 4 (45 g).

### Steps Four and Five: Synthesis of Intermediate 6

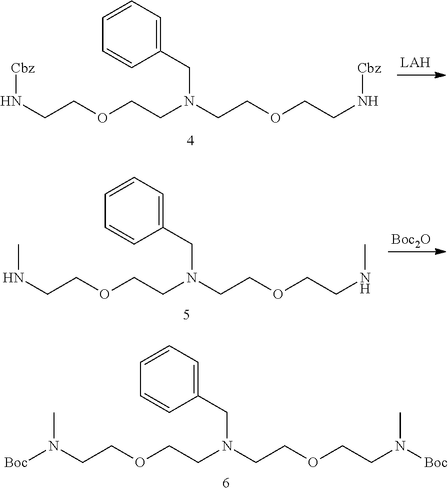

Compound 4 (43.0 g, 78.2 mmol) and tetrahydrofuran (450 mL) were added to the reaction flask and stirred at 0° C. after nitrogen displacement. LiAlH₄ (23.7 g, 62.6 mmol) was added to the reaction solution. The reaction solution was stirred at room temperature for 1.5 h, and then warmed up to reflux for 6 h with continuous stirring. The temperature was lowered to 0° C. at the end of the reaction and sodium sulfate decahydrate (90 g) was added to quench the reaction. The reaction solution was stirred at room temperature for 1 h. Triethylamine (31.7 g, 31.3 mmol) and Boc anhydride (68.3 g, 31.3 mmol) were added to the reaction solution and stirred at room temperature overnight. After the reaction completed, water (2 L) was added and ethyl acetate (300 mL×2) was used for extractions. The combined organic phases were washed with saturated saline, then dried with anhydrous sodium sulfate and concentrated to obtain the crude product. The crude product was purified by silica gel column chromatography (dichloromethane/methanol, 30/1) to give yellow oil compound 6 (20 g). LCMS: [M+1]+ found 510.2.

### Step Six: Synthesis of Inter-A

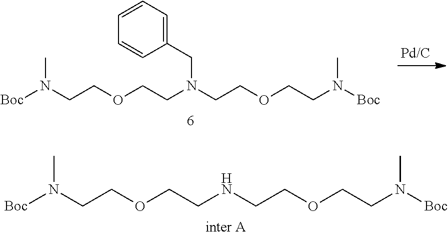

Under nitrogen atmosphere, compound 6 (19 g, 3.73 mmol), palladium carbon (2 g) and methanol (400 mL) were added to the reaction flask. Replacement of nitrogen with hydrogen were performed several times. The reaction solution was stirred overnight at room temperature. At the end of the reaction, the reaction solution was filtered, and the filter cake was washed with methanol (50 mL), and the filtrate was concentrated to obtain the crude product. The crude product was purified by silica gel column chromatography (dichloromethane/methanol, 15/1) to give a yellow oil intermediate A (14 g). LCMS: [M+1]+ found 420.2.

### Example 6: Synthesis of Linker 1

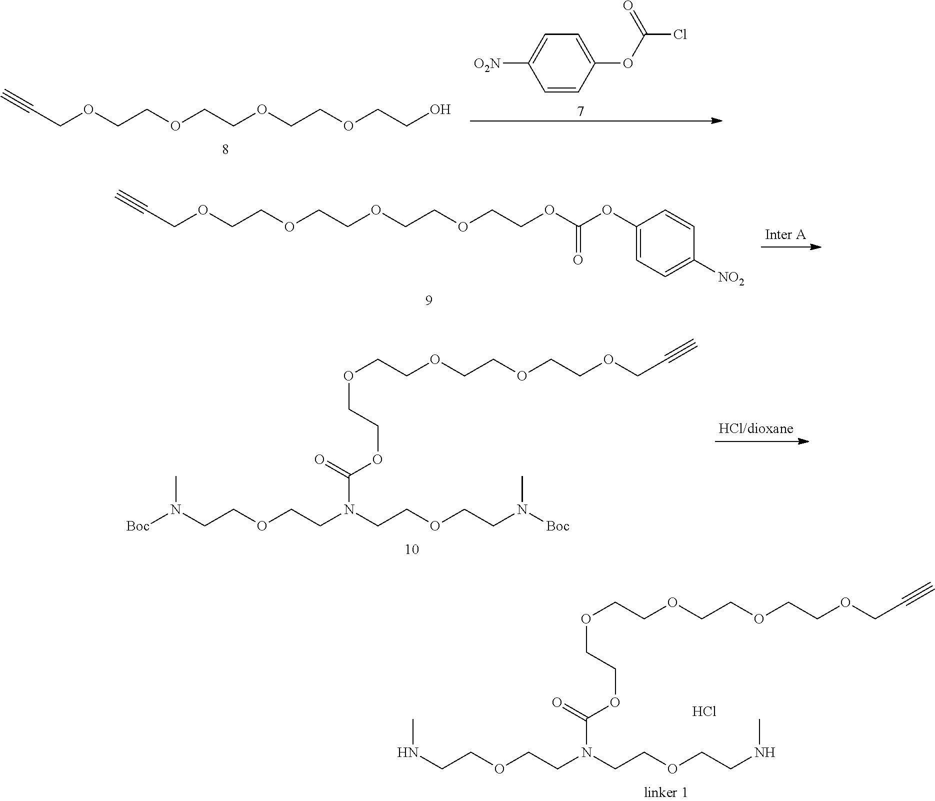 [SMILES: O=C(Cl)Oc1ccc([N+](=O)[O-])cc1 ; C#CCOCCOCCOCCOCCO ; C#CCOCCOCCOCCOCCOC(=O)Oc1ccc([N+](=O)[O-])cc1 ; C#CCOCCOCCOCCOCCOC(=O)N(CCOCCN(C)C(=O)OC(C)(C)C)CCOCCN(C)C(=O)OC(C)(C)C ; C#CCOCCOCCOCCOCCOC(=O)N(CCOCCNC)CCOCCNC ; Cl]

### Step One: Synthesis of Compound 9

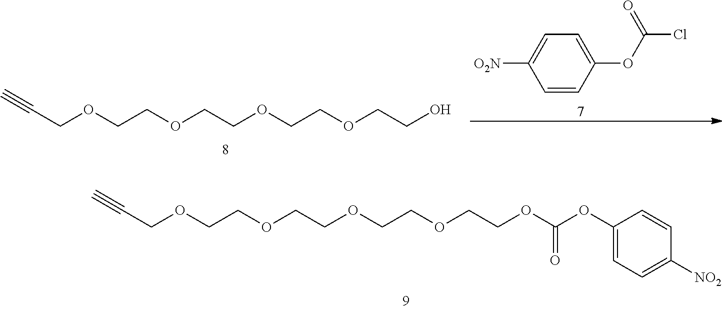 [SMILES: O=C(Cl)Oc1ccc([N+](=O)[O-])cc1 ; C#CCOCCOCCOCCOCCO ; C#CCOCCOCCOCCOCCOC(=O)Oc1ccc([N+](=O)[O-])cc1]

Compound 8 (1.5 g, 6.51 mmol) and triethylamine (1.97 g, 19.5 mmol) were dissolved in dichloromethane (30 mL) and stirred at 0° C. after nitrogen displacement. Phenyl p-nitrochloroformate (1.31 g, 6.51 mol) dissolved in dichloromethane (3 mL) was added dropwise into the reaction solution with a syringe. The reaction solution was stirred at room temperature for 6 h. The crude compound 9 was obtained by concentration at the end of the reaction.

### Step Two: Synthesis of Compound 10

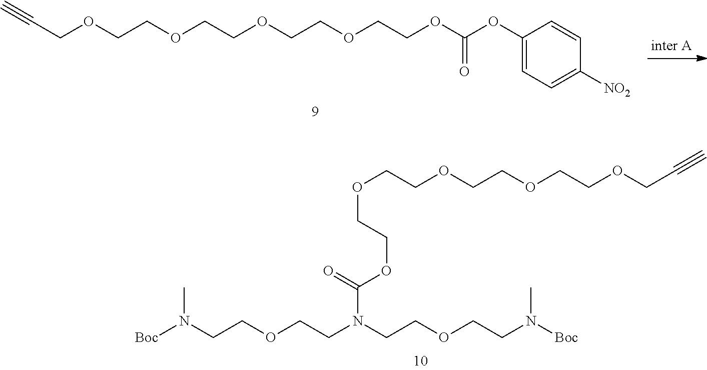 [SMILES: C#CCOCCOCCOCCOCCOC(=O)Oc1ccc([N+](=O)[O-])cc1 ; C#CCOCCOCCOCCOCCOC(=O)N(CCOCCN(C)C(=O)OC(C)(C)C)CCOCCN(C)C(=O)OC(C)(C)C]

Crude compound 9 (1.47 mmol) and triethylamine (1.97 g, 19.5 mmol) were dissolved in dichloromethane (20 mL) and stirred at room temperature. Intermediate A (2.71 g, 1.47 mol) dissolved in dichloromethane (2 mL) was added to the reaction solution. The reaction solution was stirred at room temperature overnight. The crude product was obtained by concentration at the end of the reaction. The crude product was purified by silica gel column chromatography (petroleum ether/ethyl acetate, 1/1) to give the yellow oil compound 10 (1.6 g).

### Step Three: Synthesis of Linker 1

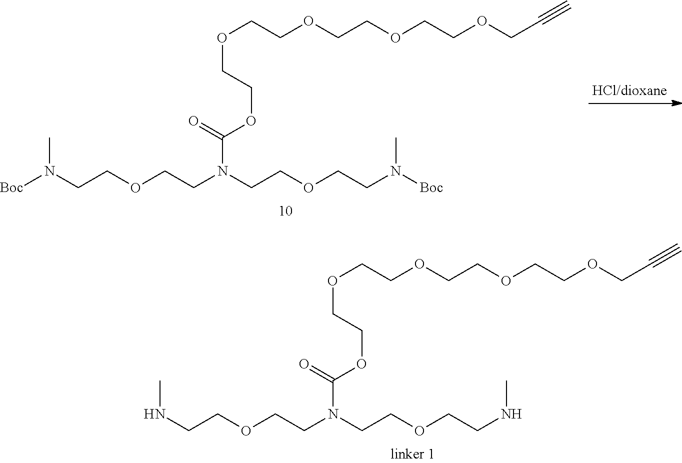

To solution of Compound 10 (1.8 g, 2.66 mmol) dissolved in 1,4-dioxane (10 mL) was added HCl (4M, 1,4-dioxane solution, 10 mL), which was stirred at room temperature for 2 h. The reaction solution was concentrated at the end of the reaction to give the crude, yellow oil linker 1 (1.6 g). LCMS: [M+1]⁺ found 478.2.

¹H NMR (400 MHz, CDCl₃): δ 9.50 (s, 4H), 4.23 (s, 4H), 3.86-3.62 (m, 18H), 3.17 (s, 3H), 2.81 (s, 5H), 2.48 (s, 1H), 1.73 (s, 6H)

### Example 7: Synthesis of Linker-2

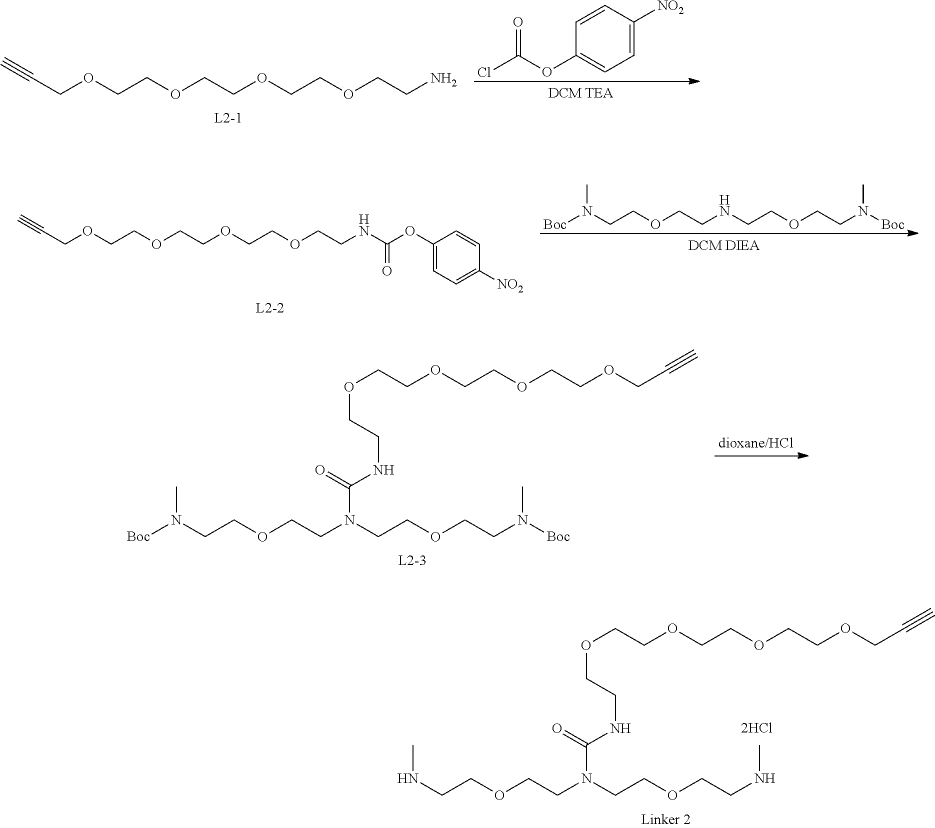 [SMILES: C#CCOCCOCCOCCOCCN ; O=C(Cl)Oc1ccc([N+](=O)[O-])cc1 ; C#CCOCCOCCOCCOCCNC(=O)Oc1ccc([N+](=O)[O-])cc1 ; CN(CCOCCNCCOCCN(C)C(=O)OC(C)(C)C)C(=O)OC(C)(C)C ; C#CCOCCOCCOCCOCCNC(=O)N(CCOCCN(C)C(=O)OC(C)(C)C)CCOCCN(C)C(=O)OC(C)(C)C ; C#CCOCCOCCOCCOCCNC(=O)N(CCOCCNC)CCOCCNC]

### Step One: Synthesis of Compound L2-2

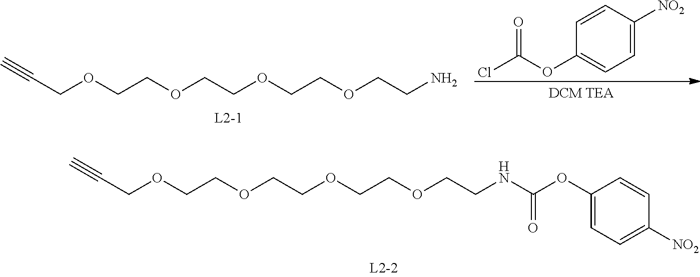 [SMILES: C#CCOCCOCCOCCOCCN ; O=C(Cl)Oc1ccc([N+](=O)[O-])cc1 ; C#CCOCCOCCOCCOCCNC(=O)Oc1ccc([N+](=O)[O-])cc1]

Compound L2-1 (2.00 g, 8.6 mmol) and triethylamine (2.61 g, 26 mmol) were dissolved in dichloromethane (20 mL) and stirred at 0° C. after nitrogen displacement. Phenyl p-nitrochloroformate (1.73 g, 8.6 mol) dissolved in dichloromethane (3 mL) was dropped into the reaction solution with a syringe. The reaction solution was stirred at 30° C. for 6 h. Water was added at the end of the reaction, and ethyl acetate was used for extraction. The combined organic phases were dried with anhydrous sodium sulfate and concentrated to obtain the crude product. The crude product was purified by silica gel column chromatography (petroleum ether/ethyl acetate, 100/1˜20/1) to obtain the yellow oil compound L2-2 (1.3 g).

### Step Two: Synthesis of Compound L2-3

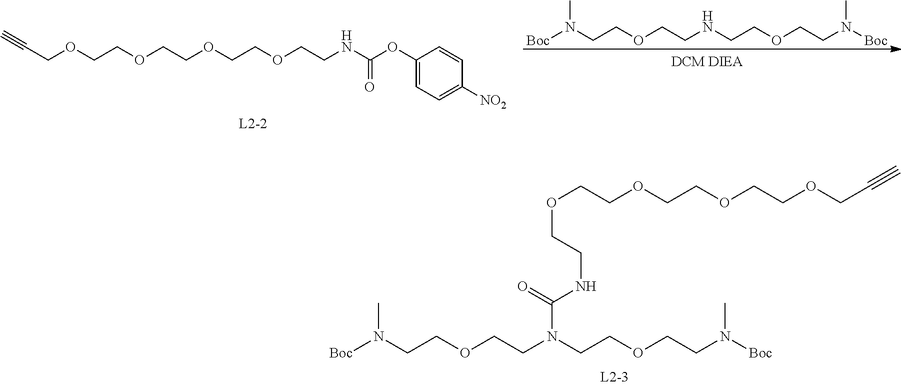 [SMILES: C#CCOCCOCCOCCOCCNC(=O)Oc1ccc([N+](=O)[O-])cc1 ; CN(CCOCCNCCOCCN(C)C(=O)OC(C)(C)C)C(=O)OC(C)(C)C ; C#CCOCCOCCOCCOCCNC(=O)N(CCOCCN(C)C(=O)OC(C)(C)C)CCOCCN(C)C(=O)OC(C)(C)C]

Compound 2 (1.3 g, 3.2 mmol) and DIEA (0.83 g, 6.4 mmol) were dissolved in acetonitrile (15 mL) and stirred at room temperature. Intermediate A (1.34 g, 3.2 mmol) dissolved in acetonitrile (5 mL) was added to the reaction solution. The reaction solution was stirred at 80° C. overnight. Water (20 mL) was added at the end of the reaction and ethyl acetate (3×20 mL) was used for extraction. The combined organic phases were dried with anhydrous sodium sulfate and concentrated to obtain the crude product. The crude product was purified by silica gel column chromatography (dichloromethane/methanol, 20/1) to give the yellow oil compound L2-3 (1.2 g).

### Step Three: Synthesis of Linker-2

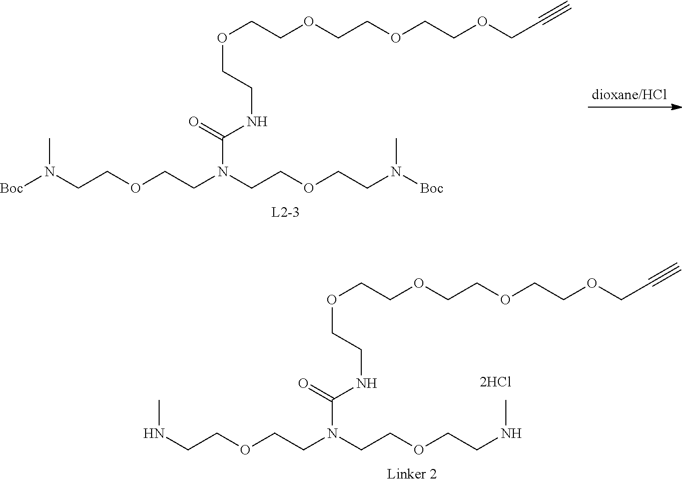

Compound L2-3 (1.2 g, 1.8 mmol) dissolved in 1,4-dioxane (10 mL) was added with HCl (4M, 1,4-dioxane solution, 10 mL), which was stirred at room temperature for 2 h. The reaction solution was concentrated at the end of the reaction to give the crude brown oil Linker 2 (0.93 g). LCMS: [M+1]+ found 477.3.

¹H NMR (400 MHz, DMSO_d₆) 8.94 (s, 4H), 6.41 (m, 1H), 4.14 (s, 2H), 3.65-3.67 (m, 4H), 3.54 (m, 6H), 3.51 (m, 4H), 3.44-3.34 (m, 11H), 3.16-3.18 (m, 2H), 3.06 (m, 4H), 2.54 (s, 6H).

### Example 8: Synthesis of Linker-4

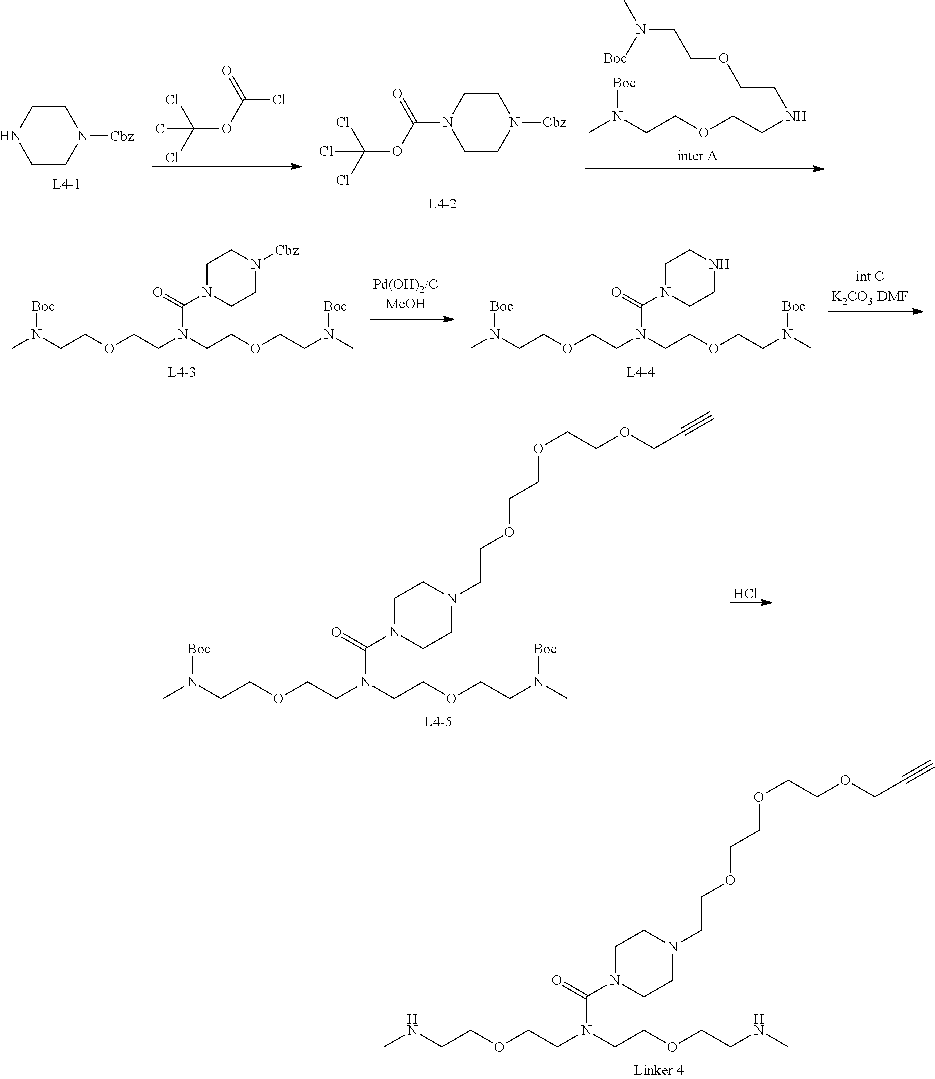

### Step One: Synthesis of L4-2

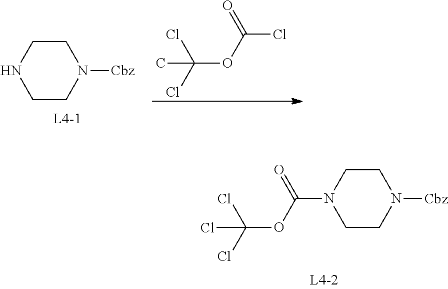

Trichloromethyl chloroformate (539 mg, 1.02 mol) dissolved in dry tetrahydrofuran (5 mL) was stirred at 0° C. after nitrogen displacement. Compound L4-1 (500 mg, 2.27 mmol) and DIEA that were dissolved in tetrahydrofuran (5 mL) was added dropwise to the reaction solution. The reaction solution was stirred at 0° C. for 1 h. At the end of the reaction, the filtrate obtained from filtration through diatomaceous earth was concentrated to give the crude compound L4-2. The crude compound L4-2 was used directly in the next step without purification.

### Step Two: Synthesis of L4-3

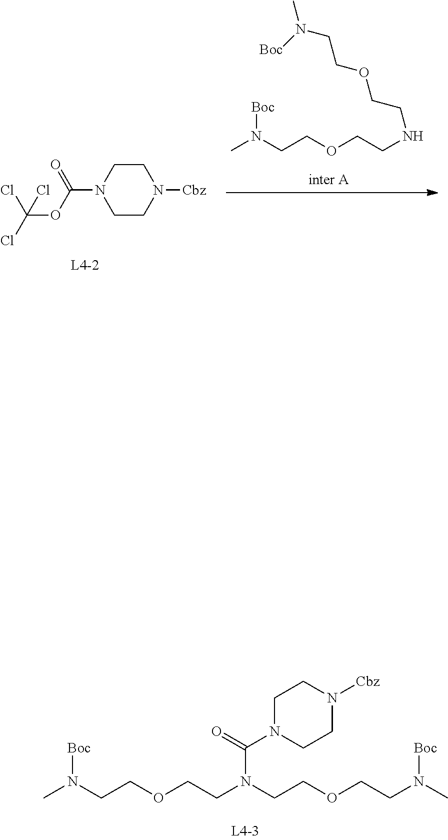

Intermediate A (300 mg, 0.715 mmol) and DIEA (277 mg, 2.15 mmol) that were dissolved in dichloromethane (2 mL) was stirred at 0° C. The crude compound L4-2 dissolved in dichloromethane (1 mL) was added dropwise to the reaction solution. The reaction solution was stirred overnight at room temperature. At the end of the reaction, the pH was adjusted to 7 with 1N hydrochloric acid and dichloromethane (2×1 mL) was used for extraction. The organic phases were combined and concentrated to obtain the crude product. The crude product was purified by preparative silica gel plate to give the yellow oil compound L4-3 (150 mg). LCMS: [M+1]+ found 666.4.

### Step Three: Synthesis of L4-4

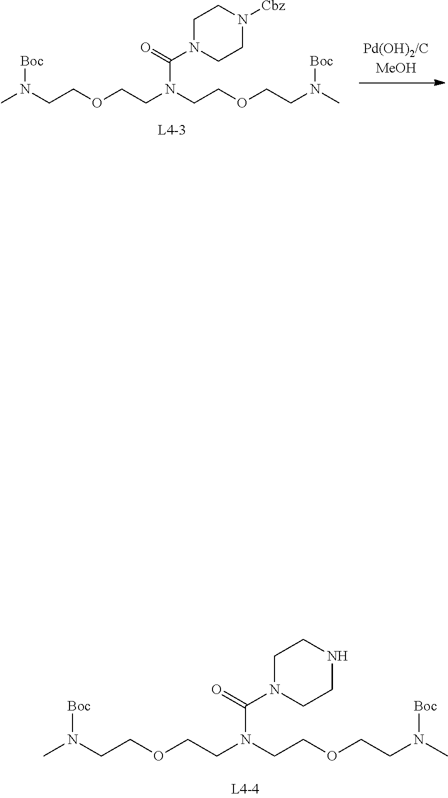

Compound L4-3 (3 g, 4.51 mmol), palladium/carbon hydroxide (0.3 g) and methanol (60 mL) were added to the reaction flask under nitrogen atmosphere. Hydrogen was used to displace nitrogen several times. The reaction solution was stirred overnight at room temperature. At the end of the reaction, the reaction solution was filtered, and the filter cake was washed with methanol (10 mL). The filtrate was concentrated to give the crude yellow oil compound L4-4 (2.4 g). LCMS: [M+1]+ found 532.3

### Step Four: Synthesis of L4-5

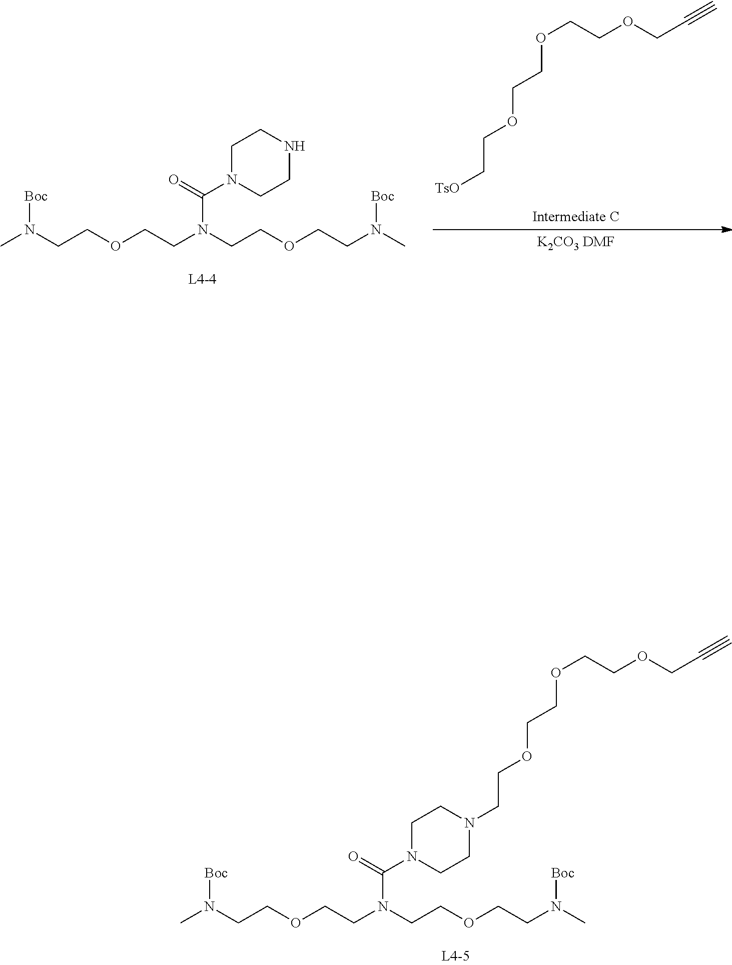

Compound L4-4 (2.4 g, 4.51 mmol), DIEA (1.74 g, 13.4 mmol) and Intermediate C (3.08 g, 9.02 mmol) were dissolved in DMF (20 mL) and stirred at 80° C. overnight after the nitrogen displacement. At the end of the reaction, the solution was reduced to room temperature, added with water (100 mL) and extracted with ethyl acetate (2×20 mL). The combined organic phases were dried with anhydrous sodium sulfate and concentrated to obtain the crude product. The crude product was purified by silica gel column chromatography (dichloromethane/methanol, 20/1) to obtain the crude product. The crude product was purified three more times with preparative silica gel plates to give the yellow oil compound L4-5 (350 mg). LCMS: [M+1]+ found 702.3.

### Step Five: Synthesis of Linker 4

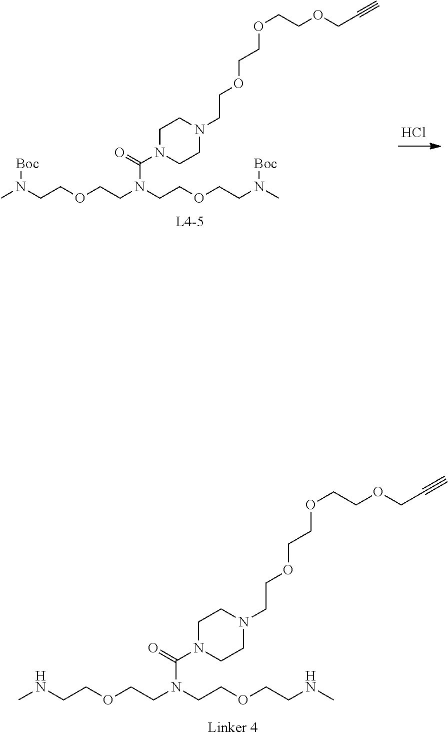

Compound L4-5 (350 mg, 2.66 mmol) dissolved in 1,4-dioxane (3 mL) was added with HCl (4M, 1,4-dioxane solution, 5 mL) and stirred at room temperature for 2 h. The reaction was concentrated at the end of the reaction to give the crude yellow oil Linker 4 (310 mg).

LCMS: [M+1]⁺ found 502.3, ¹H NMR (400 MHz, CDCl₃): δ 12.19-12.17 (m, 1H), 9.53-9.48 (m, 4H), 4.26-4.22 (m, 2H), 4.06-4.15 (m, 31H), 3.07-2.70 (m, 9H), 2.53-2.27 (m, 6H), 2.18 (s, 1H), 1.27-1.24 (m, 4H).

### Example 9: Synthesis of Linker 5

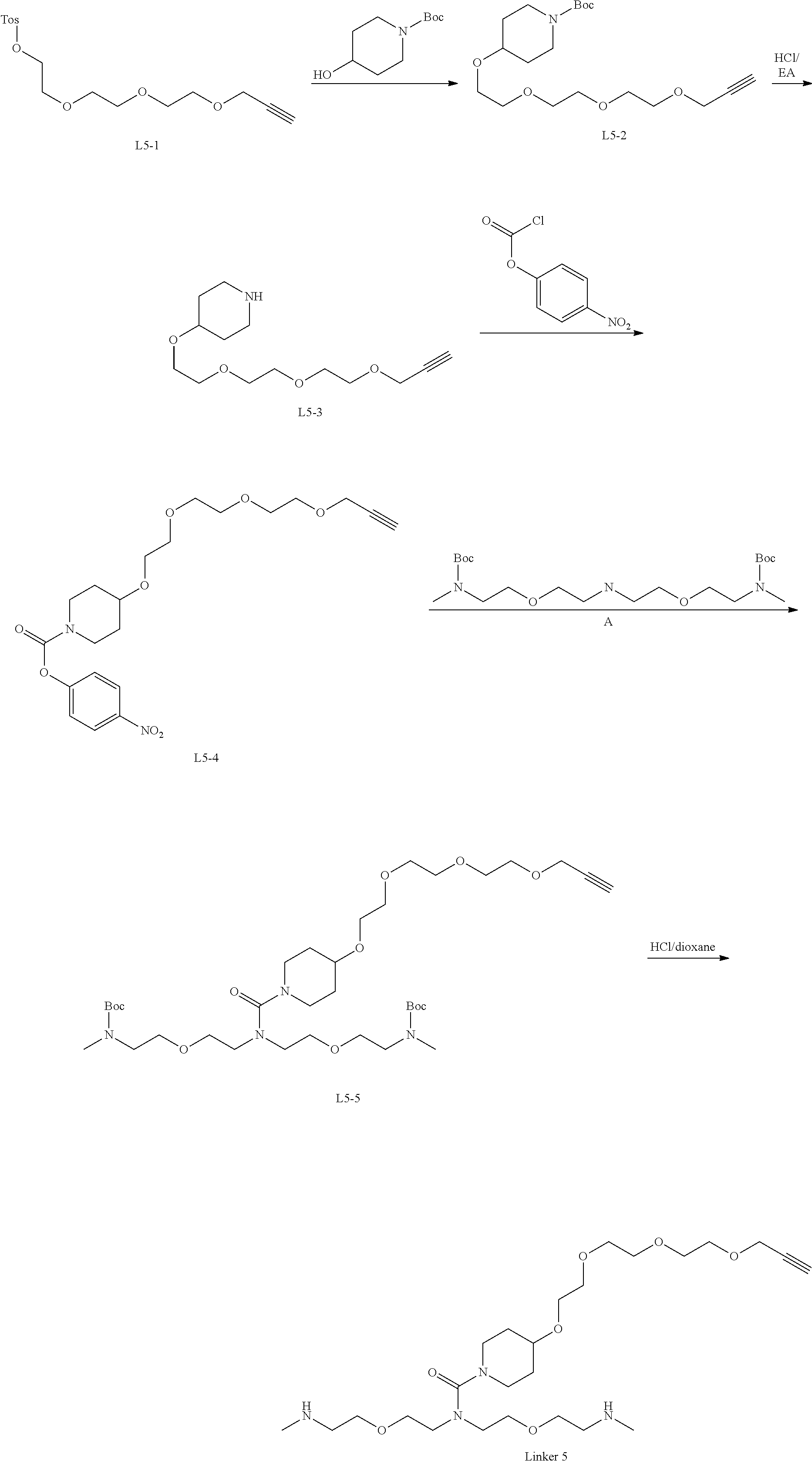

### Step One: Synthesis of L5-2

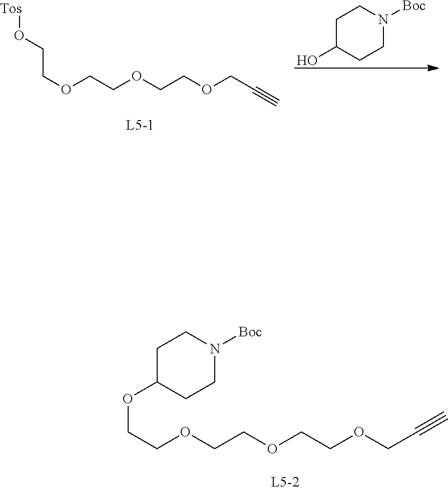

N-Boc-4-hydroxypiperidine (5.0 g, 14.6 mmol) and DMF (50 mL) were added to the reaction flask and stirred at 0° C. after the nitrogen displacement. Sodium hydrogen (0.64 g, 16 mmol) was added to the reaction solution. The reaction solution was stirred at room temperature for 0.5 h. Compound 1 (3.2 g, 16 mmol) was then added to the reaction solution and continued to be stirred at room temperature for 2 h. At the end of the reaction, the reaction solution was added to ice water and extracted with ethyl acetate. The combined organic phases were washed with saturated brine, dried with anhydrous sodium sulfate and concentrated to obtain the crude compound L5-2 (4.5 g).

### Step Two: Synthesis of L5-3

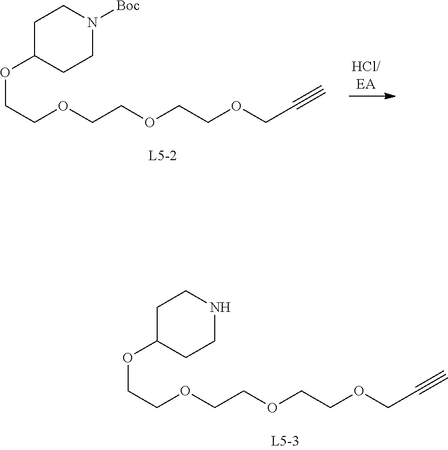 [SMILES: C#CCOCCOCCOCCOC1CCN(C(=O)OC(C)(C)C)CC1 ; C#CCOCCOCCOCCOC1CCNCC1]

Compound L5-2 (4.5 g, 12.1 mmol) dissolved in ethyl acetate (30 mL) was added with HCl (4M, ethyl acetate, 30 mL) and stirred at room temperature for 2 h. After the reaction was completed, reaction solution was concentrated to give the crude compound L5-3 (5.0 g). LCMS: [M+1]+ found 272.2.

### Step Three: Synthesis of L5-4

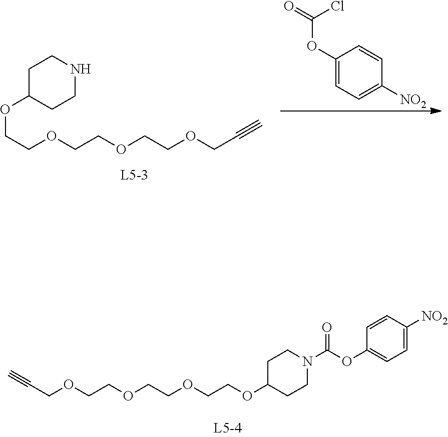

Compound L5-3 (5.00 g, crude) and triethylamine (5.6 g, 55.2 mmol) that were dissolved in dichloromethane (50 mL) was stirred at room temperature after the nitrogen displacement. Phenyl p-nitrochloroformate (5.6 g, 27.6 mmol) dissolved in dichloromethane (10 mL) was dropped into the reaction solution with a syringe. The reaction solution was stirred at room temperature for 2 h. At the end of the reaction, ice water was added and dichloromethane was used for extraction. The combined organic phases were dried with anhydrous sodium sulfate and concentrated to obtain the crude product. The crude product was purified by silica gel column chromatography to obtain the yellow oil compound L5-4 (5.2 g, crude). LCMS: [M+1]+ found 437.1.

### Step Four: Synthesis of L5-5

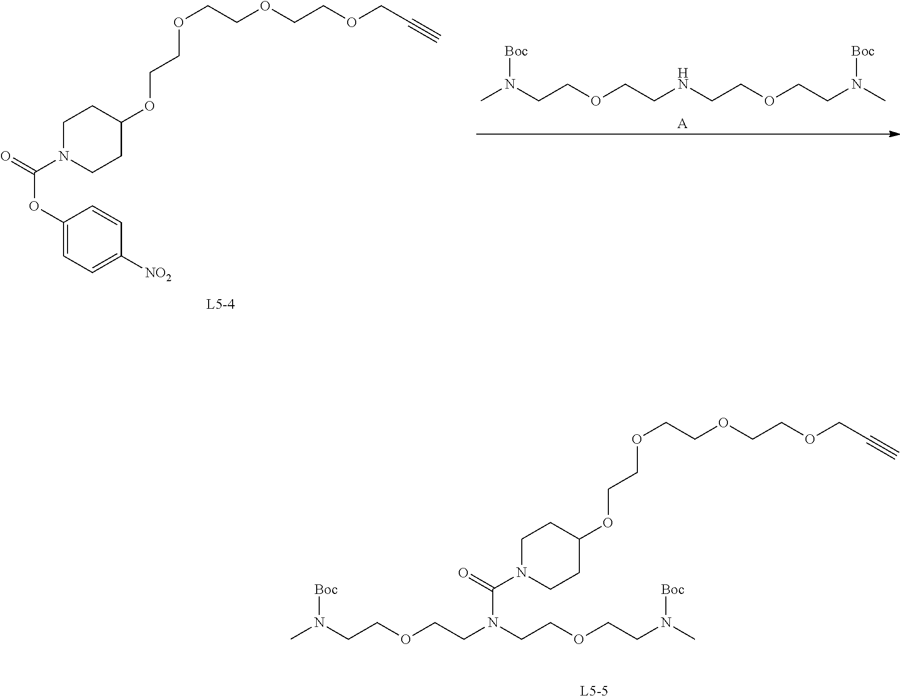

Compound L5-4 (5.2 g, crude) and potassium carbonate (1.91 g, 13.8 mmol) were dissolved in DMF (20 mL) and stirred at room temperature. Intermediate A (1.94 g, 4.6 mmol) was dissolved in DMF (5 mL) and added to the reaction solution. The reaction solution was stirred at 130° C. for 48 h. At the end of the reaction, water (200 mL) was added and ethyl acetate (3×100 mL) was used for extraction. The combined organic phases were dried with anhydrous sodium sulfate and concentrated to obtain the crude product. The crude product was purified by silica gel column chromatography to obtain the yellow oil compound L5-5 (0.8 g). LCMS: [M+1]+ found 717.5.

### Step Five: Synthesis of Linker 5

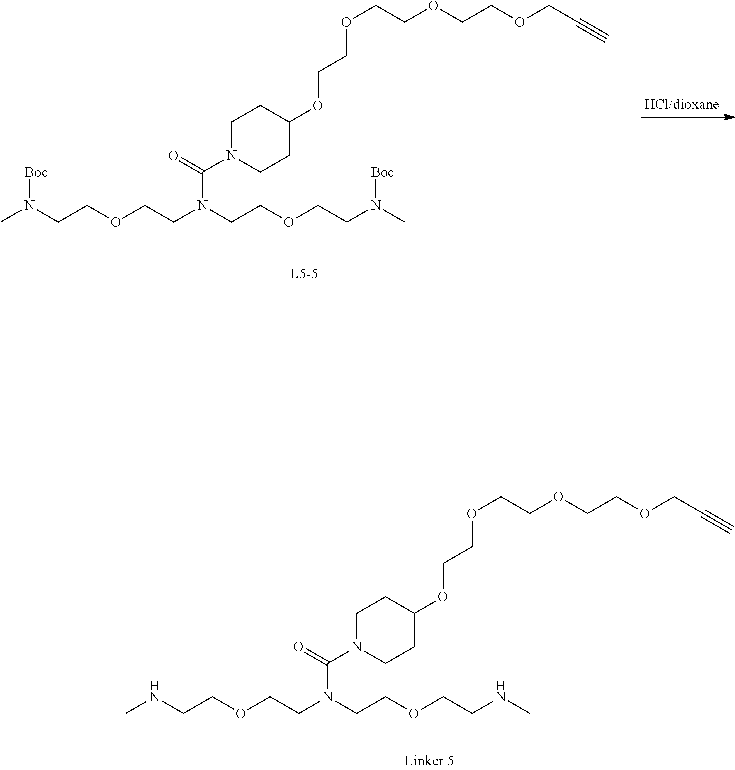

Compound L5-5 (0.8 g, 1.1 mmol) dissolved in ethyl acetate (10 mL) was added with HCl (4M, ethyl acetate, 10 mL) and stirred at room temperature for 2 h. After the reaction was completed, the reaction solution was concentrated to give the crude compound Linker 5 (0.61 g). LCMS: [M+1]+ found 517.3.

¹H NMR (400 MHz, CDCl₃): δ 9.63 (s, 4H), 4.23 (d, 2H), 3.95 (s, 4H), 3.75 (s, 3H), 3.73-3.65 (m, 17H), 3.26-3.14 (m, 6H), 2.79 (m, 9H), 2.48 (s, 1H), 2.02 (d, 2H), 1.72 (s, 2H).

### Example 10: Synthesis of Intermediate Inter B

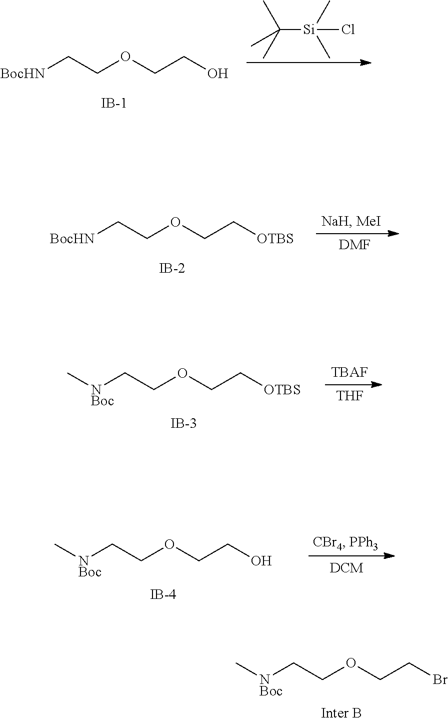

### Step One: Synthesis of IB-2

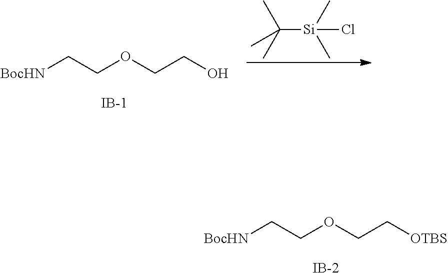

2-(2-BOC-aminoethoxy)ethanol (IB-1, 9.03 g, 44.0 mmol) and imidazole (6.58 g, 96.8 mmol) were dissolved in dichloromethane (100 mL), which was added drop-wise with a solution of tert-butyldimethylchlorosilane (7.30 g, 48.4 mmol) dissolved in dichloromethane (30 mL) and was stirred for 16 h at room temperature. After the reaction was completed, dichloromethane (100 mL) was added, and dilute hydrochloric acid (0.5 M, 200 mL*2) and saturated sodium bicarbonate (200 mL*2) was used sequentially for washing. The organic phase was dried with anhydrous sodium sulfate, concentrated under reduced pressure and purified by silica gel column chromatography (PE:EA=3:1) to give the white solid IB-2 (13.5 g).

### Step Two: Synthesis of IB-3

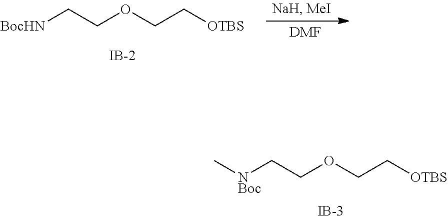

IB-2 (13.5 g, 42.3 mmol) was dissolved in DMF (100 mL), and sodium hydrogen (6.77 g, 169 mmol) was added under an ice bath. After stirring for 20 min, iodomethane (13 mL, 212 mmol) was added followed by stirring for 16 h at room temperature. After the reaction was completed, water (100 mL) and ethyl acetate (200 mL) were added, and the organic phase was dried over anhydrous sodium sulfate and concentrated under reduced pressure to give the crude IB-3 (17.0 g), which was directly used in the next step.

### Step Three: Synthesis of IB-4

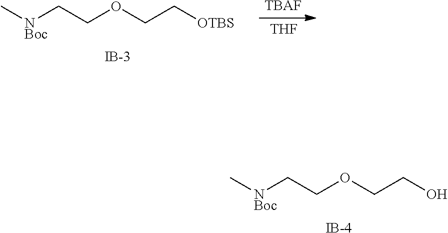

IB-3 (17.0 g, calculated as 42.3 mmol) and tetrabutylammonium fluoride trihydrate (29.34 g, 93.0 mmol) were dissolved in tetrahydrofuran (100 mL) and stirred at room temperature for 1 hour. After the reaction was completed, water (200 mL) was added and ethyl acetate (100 mL*3) was used for extraction, followed by washing with saturated brine (100 mL*2). The organic phase was dried with anhydrous sodium sulfate, concentrated under reduced pressure and then purified by silica gel column chromatography (PE:EA=2:1) to give the brown oil IB-4 (5.6 g). LCMS[M-100]+: 120.15

### Step Four: Synthesis of Inter B

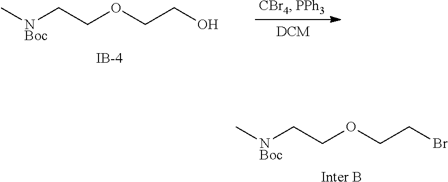

IB-4 (13.0 g, 59.32 mmol) and carbon tetrabromide (29.5 g, 88.98 mmol) were dissolved in dichloromethane (50 mL), to which the solution of triphenylphosphine (23.34 g, 88.98 mmol) in dichloromethane (20 mL) was added dropwise under an ice bath. The reaction solution was stirred at room temperature for 24 hours. After the reaction was completed, concentration under reduced pressure and purification by silica gel column chromatography (PE:EA=2:1) were performed to give the colorless oil IB-4 (6.5 g). LCMS[M-100]+:182.17.

### Example 11: Synthesis of Linker 8

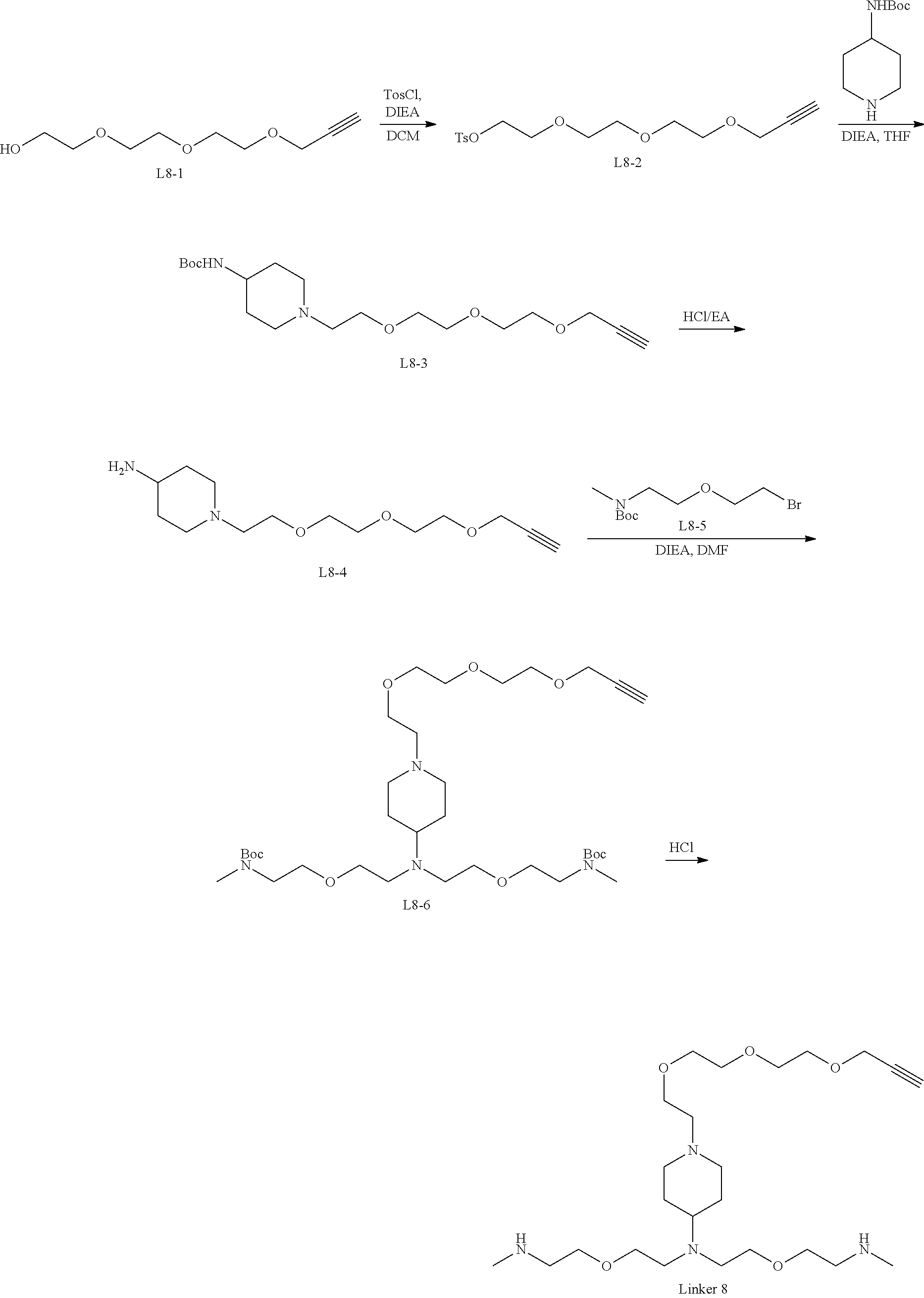

### Step One: Synthesis of L8-2

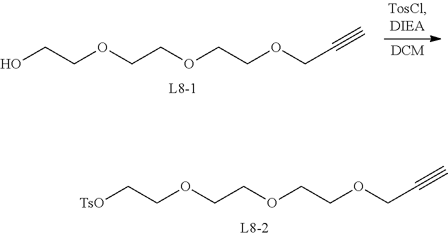 [SMILES: C#CCOCCOCCOCCO ; C#CCOCCOCCOCCOS(=O)(=O)c1ccc(C)cc1]

After propynyl-triethylene glycol (L8-1, 5.0 g, 26.56 mmol) was dissolved in dichloromethane (50 mL), N,N-diisopropylethylamine (6.87 g, 53.13 mmol) and p-toluenesulphonyl chloride (5.57 g, 29.22 mmol) were added, and the reaction was stirred at room temperature for 10 hours. After the reaction completed, water (50 mL) was added for washing, and the organic phase was dried over anhydrous sodium sulfate and concentrated under reduced pressure to give crude L8-2 (11.0 g), which was directly used in the next step. LCMS[M+1]+:343.23.

### Step Two: Synthesis of L8-3

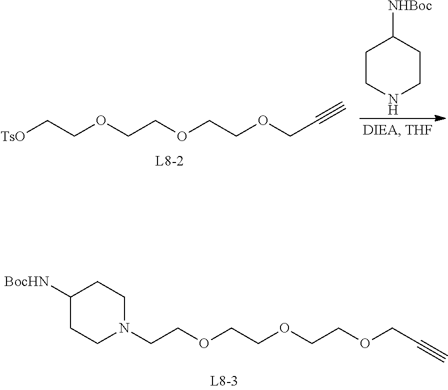

After L8-2 (11.0 g, calculated as 26.56 mmol) was dissolved in tetrahydrofuran (50 mL), 4-tert-butoxycarbonylaminopiperidine (5.3 g, 26.56 mmol) and N,N-diisopropylethylamine (6.9 g, 53.12 mmol) were added, and stirred at 70° C. for 24 h. After the reaction was completed, concentration under reduced pressure and purification by silica gel column chromatography (EA) were performed to give the brown oil L8-3 (5.1 g). LCMS[M+1]+: 371.40

### Step Three: Synthesis of L8-4

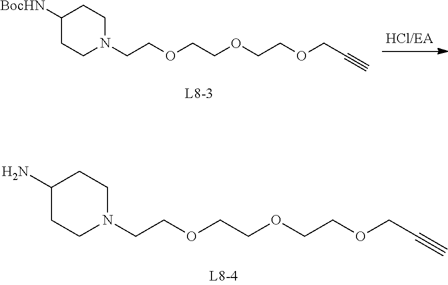 [SMILES: C#CCOCCOCCOCCN1CCC(NC(=O)OC(C)(C)C)CC1 ; C#CCOCCOCCOCCN1CCC(N)CC1]

L8-3 (3.43 g, 9.27 mmol) placed in the solution (30 ml) of ethyl acetate and hydrochloride was stirred at room temperature for 1 h. At the end of the reaction, concentration was performed to give the crude L8-4 (2.5 g) that was directly used in the next step. LCMS[M+1]+: 271.30

### Step Four: Synthesis of L8-6

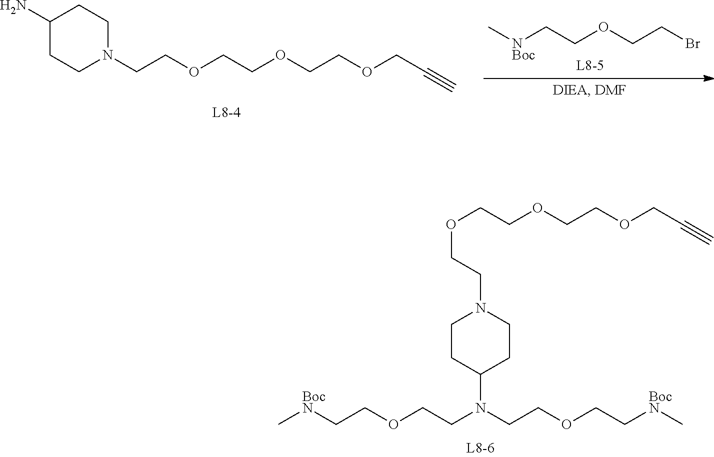

Crude L8-4 (2.5 g, calculated as 9.27 mmol) was dissolved in DMF (50 ml), and then L8-5 (6.5 g, 23.1 mmol) and DIEA (2.4 g, 18.5 mmol) were added and stirred at 75° C. for 24 h. Concentration under reduced pressure, silica gel column chromatography (DCM:MeOH=10:1) and reverse HPLC were performed to give the L8-6 (90 mg). LCMS[M+1]+:673.60.

### Step Five: Synthesis of Linker 8

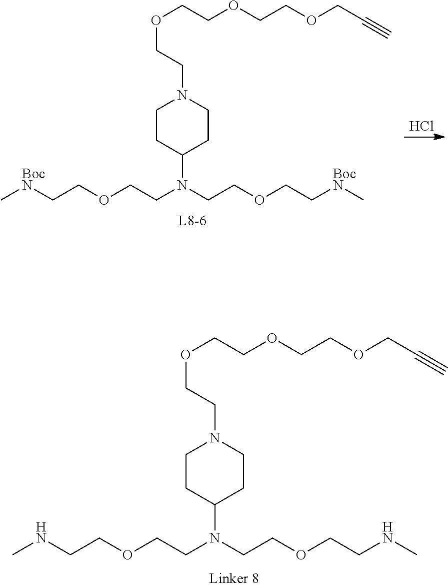

L8-6 (90 mg, 0.15 mmol) was placed in 1,4-dioxane hydrochloride solution (5 ml) and stirred at room temperature for 1 h. At the end of the reaction, concentration was performed to give the crude Linker 8 (80 mg).

### Example 12: Synthesis of Linker 9

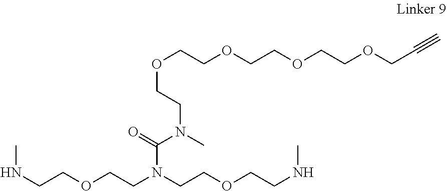 [SMILES: C#CCOCCOCCOCCOCCN(C)C(=O)N(CCOCCNC)CCOCCNC]

### Step One: Synthesis of L9-2

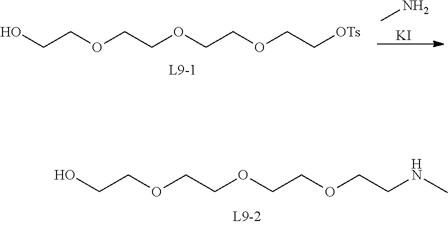

L9-1 (3.2 g, 9.17 mmol) was added to the reaction flask, followed by the addition of methanol solution of methylamine (10 mL) and KI (304 mg, 1.83 mmol). The solution was stirred at room temperature overnight. At the end of the reaction, the solvent was removed by rotary evaporation to obtain the crude L9-2, which was used directly in the next step.

### Step Two: Synthesis of L9-3

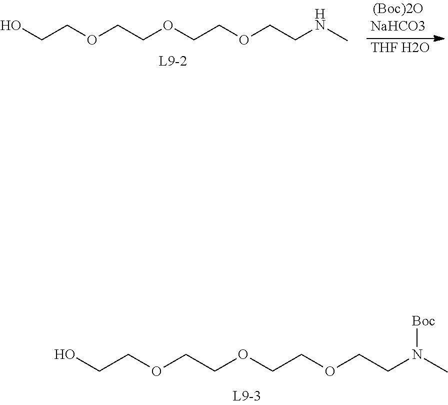 [SMILES: "THF H2O" ; CNCCOCCOCCOCCO ; CN(CCOCCOCCOCCO)C(=O)OC(C)(C)C]

The crude compound L9-2 (1.9 g, 9.18 mmol) was dissolved in a mixed solvent of tetrahydrofuran:water (20 mL:10 mL), followed by the additions of sodium bicarbonate (1.5 g, 18.36 mmol) and di-tert-butyl dicarbonate (2.2 g, 10.1 mmol), which was stirred at room temperature overnight. At the end of the reaction, the reaction solution was quenched by addition of water (30 mL), extracted with ethyl acetate (50 mL*3), and dried with anhydrous sodium sulfate. The organic phase was concentrate by column purification (PE:EA, EA=30%) to give the colorless liquid compound L9-3 (1.5 g).

### Step Three: Synthesis of L9-4

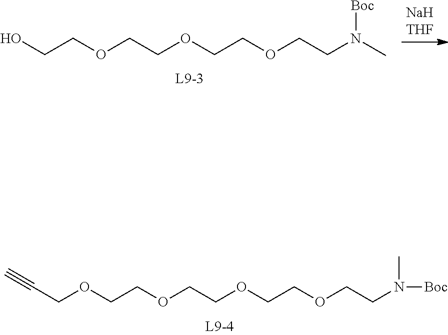

Compound L9-3 (1.5 g, 4.88 mmol) was dissolved in tetrahydrofuran (15 mL) followed by addition of sodium hydride (290 mg, 7.33 mmol, 60%) at 0° C. The reaction solution was stirred at 0° C. for 30 min. Bromopropargyl (700 mg, 5.86 mmol) was added, and the reaction was carried out overnight at room temperature. At the end of the reaction, the reaction solution was quenched by addition of water (20 mL), extracted with ethyl acetate (20 mL), extracted with ethyl acetate (50 mL*3), dried with anhydrous sodium sulfate, concentrated, and purified by column (PE:EA, EA=40%) to give the sticky, colorless product L9-4 (1.0 g).

### Step Four: Synthesis of L9-5

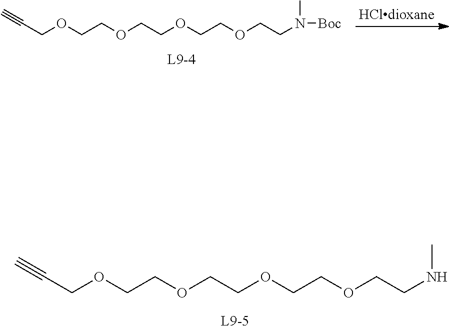 [SMILES: C#CCOCCOCCOCCOCCN(C)C(=O)OC(C)(C)C ; C#CCOCCOCCOCCOCCNC]

Compound L9-4 (600 mg, 1.73 mmol) was dissolved in dichloromethane (5 mL), and a solution of hydrochloride in dioxane (5 mL, 4M in dioxane) was added. The reaction was carried out at room temperature for 1 h. At the end of the reaction, a direct concentration was performed to give the crude L9-5 in the form of hydrochloride, which was used directly in the next step.

### Step Five: Synthesis of L9-6

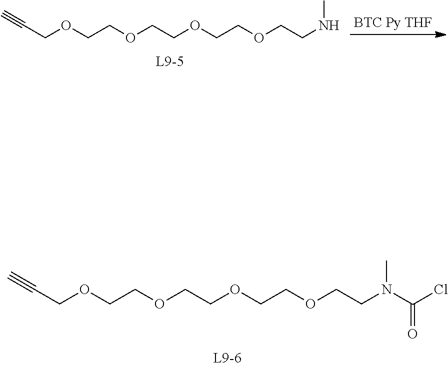

Triphosgene (300 mg, 0.98 mmol) was dissolved in tetrahydrofuran (15 mL), to which pyridine (250 mg, 2.94 mmol) in tetrahydrofuran solution (2 mL) was added in ice-water bath under nitrogen atmosphere. The solution was stirred in ice-water bath for 30 min. Then, compound L9-5 (550 mg, 1.96 mmol), triethylamine (300 mg, 2.94 mmol) in tetrahydrofuran solution (2 mL) were added, which reacted at room temperature for 3 h. After the reaction completed, the reaction was quenched with water (10 mL), extracted with ethyl acetate (30 mL*3), dried with anhydrous sodium sulfate and concentrated to obtain the crude L9-6, which was directly used in the next step.

### Step Six: Synthesis of L9-7

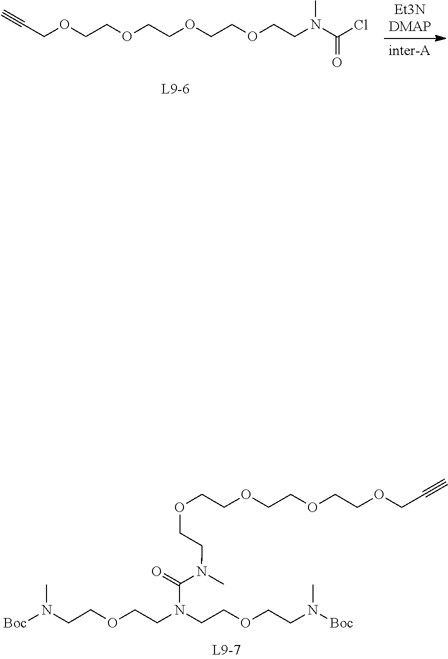 [SMILES: C#CCOCCOCCOCCOCCN(C)C(=O)Cl ; C#CCOCCOCCOCCOCCN(C)C(=O)N(CCOCCN(C)C(=O)OC(C)(C)C)CCOCCN(C)C(=O)OC(C)(C)C]

Compound L9-6 was dissolved in tetrahydrofuran (15 mL), followed by addition of Inter-A (500 mg, 1.17 mmol) in tetrahydrofuran (2 mL), triethylamine (300 mg, 2.94 mmol), and 4-dimethylaminopyridine (20 mg, 0.16 mmol) in tetrahydrofuran (2 mL), and the reaction was carried out for 4 h at 60° C., and stirred at room temperature overnight. After the reaction completed, the reaction was quenched with water (10 mL), extracted with ethyl acetate (30 mL*3), dried with anhydrous sodium sulfate and concentrated to give the compound L9-7 (530 mg).

### Step Seven: Synthesis of L9-8

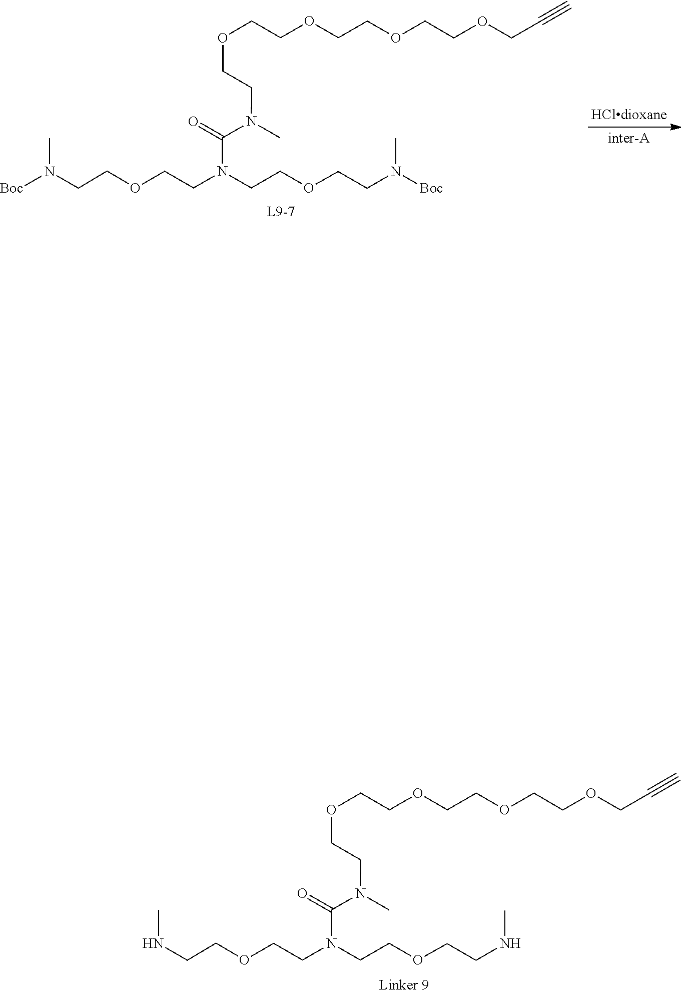

Compound L9-7 (100 mg, 0.14 mmol) was dissolved in dichloromethane (5 mL), followed by addition of hydrochloride in dioxane solution (5 mL, 4M in dioxane). The reaction was carried out at room temperature for 1 h. A direct concentration was performed to give the crude Linker 9 in the form of hydrochloride, which was used directly in the next step. LCMS[M+1]+: 491.3

### Example 13: Synthesis of Linker 10

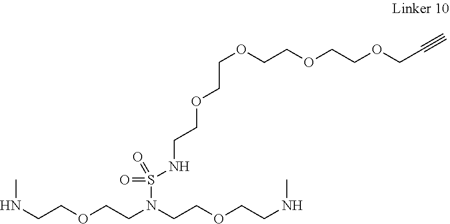 [SMILES: C#CCOCCOCCOCCOCCNS(=O)(=O)N(CCOCCNC)CCOCCNC]

### Step One: Synthesis of L10-2

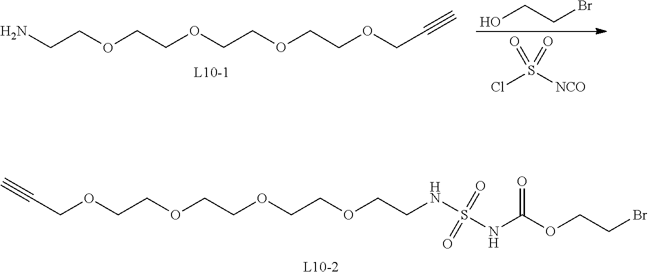

Chlorosulfonyl isocyanate (400 mg, 2.83 mmol) was dissolved in dichloromethane (10 mL) and stirred at 0° C. under nitrogen protection. A solution of bromoethanol (353 mg, 2.83 mmol) in dichloromethane (5 mL) was added dropwise and stirred at 0° C. for 1 h. Then, a solution of L10-1 (2.83 mmol) in dichloromethane (5 mL) was added dropwise and stirred at 0° C. for 1 h. After the reaction was completed, dichloromethane (10 mL) was added, and the diluted hydrochloric acid (1M, 20 mL) was used for washing. The organic phase was dried over anhydrous sodium sulfate, and the solvent was removed by rotary evaporation. The crude yellow oil L10-2 (800 mg) was obtained by purification of silica gel column chromatography (PE:EA=1:1).

### Step Two: Synthesis of L10-3

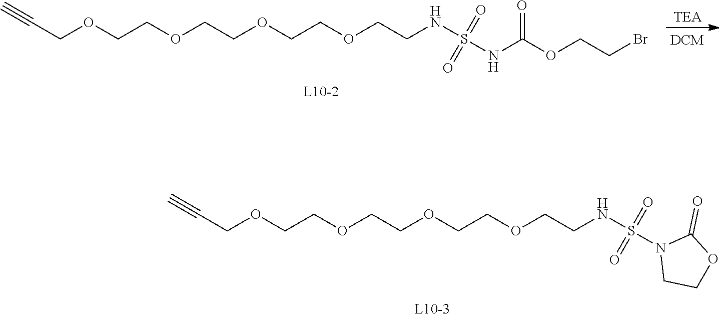 [SMILES: C#CCOCCOCCOCCOCCNS(=O)(=O)NC(=O)OCCBr ; C#CCOCCOCCOCCOCCNS(=O)(=O)N1CCOC1=O]

The crude L10-2 (800 mg) was dissolved in dichloromethane (10 mL), and TEA (2 mL) was added. The reaction solution was stirred at room temperature for 16 hours. After the reaction was completed, dichloromethane (10 mL) was added, and dilute hydrochloric acid (1M, 20 mL) was used for washing. The organic phase was dried over anhydrous sodium sulfate, and the solvent was removed by rotary evaporation. The yellow oil L10-3 (670 mg) was obtained by purification of the silica gel column chromatography (PE:EA=1:3).

### Step Three: Synthesis of L10-4

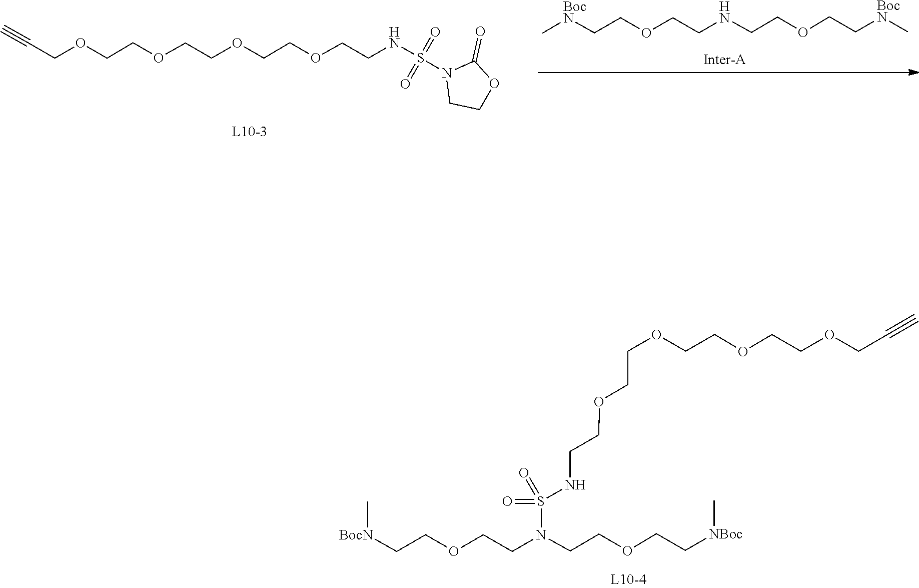

L10-3 (300 mg, 0.79 mmol), Inter-A (330 mg, 0.79 mmol) and TEA (160 mg, 1.60 mmol) were dissolved in acetonitrile (10 mL) and stirred for 10 h at 80° C. After the reaction was completed, the solvent was removed by rotary evaporation and purification by the reversed-phase column chromatography (acetonitrile/water) was performed to give the yellow oil L10-4 (190 mg).

### Step Four: Synthesis of Linker 10

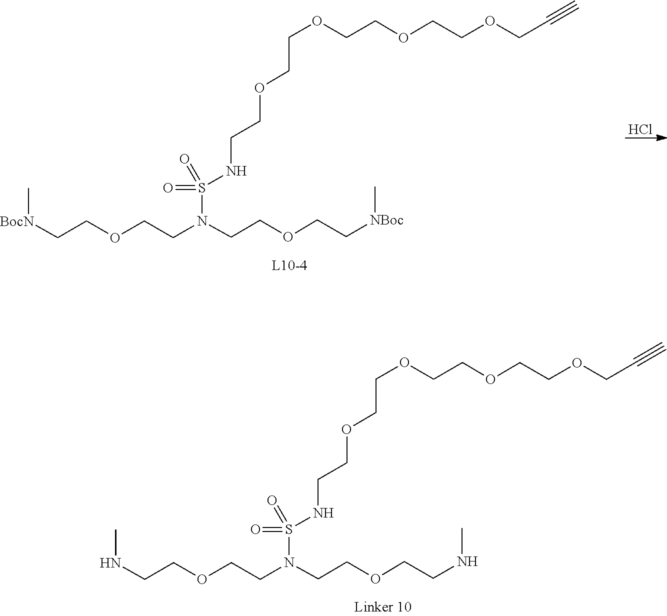

L10-4 (107 mg, 0.15 mmol) was placed in 1,4-dioxane hydrochloride solution (5 ml) and stirred at room temperature for 1 h. At the end of the reaction, concentration was performed to obtain the crude Linker 10 that was directly used in the next step. MS[M+1]⁺: 513.51.

### Example 14: Synthesis of Linker 11

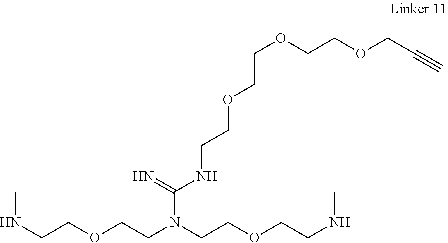 [SMILES: C#CCOCCOCCOCCNC(=N)N(CCOCCNC)CCOCCNC]

### Step One: Synthesis of L11-4

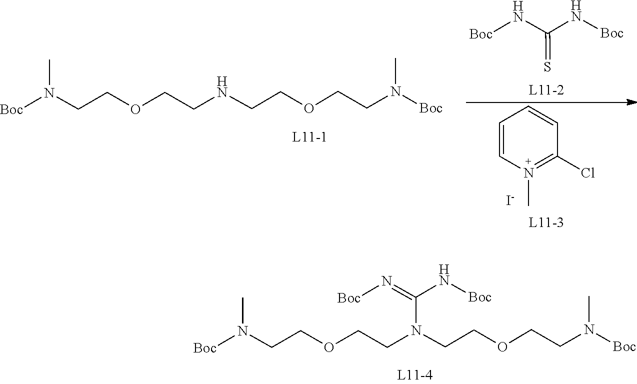

L11-1 (360 mg, 0.86 mmol), L11-2 (238 mg, 0.86 mmol), L11-3 (230 mg, 0.9 mmol) and triethylamine (93 mg, 0.9 mmol) were dissolved in dichloromethane (10 mL) under nitrogen protection and were stirred for 3 h at room temperature. After the reaction completed, the solvent was removed by rotary evaporation and purification by silica gel column chromatography (PE:EA=1:1) was obtained to give colorless oil L11-4 (830 mg).

### Step Two: Synthesis of L11-6

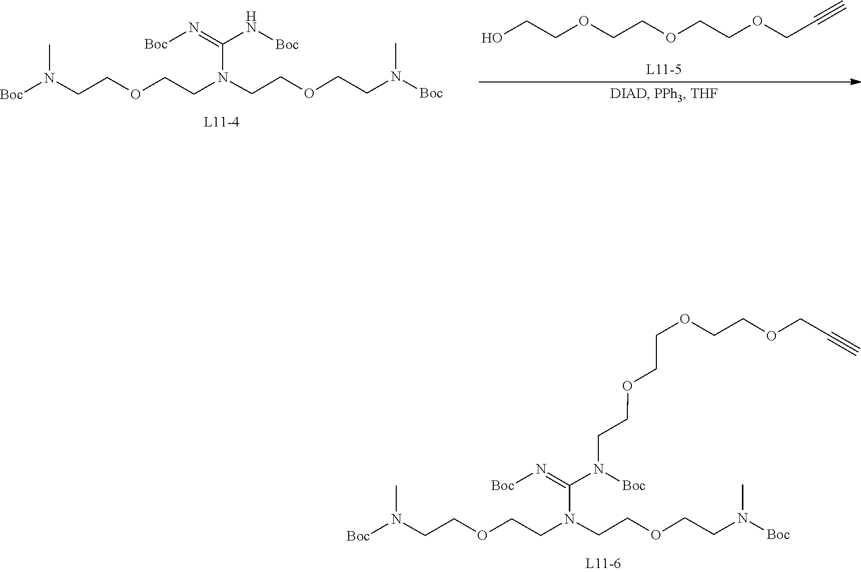 [SMILES: CN(CCOCCN(CCOCCN(C)C(=O)OC(C)(C)C)/C(=N\C(=O)OC(C)(C)C)NC(=O)OC(C)(C)C)C(=O)OC(C)(C)C ; C#CCOCCOCCOCCO ; C#CCOCCOCCOCCN(C(=O)OC(C)(C)C)/C(=N/C(=O)OC(C)(C)C)N(CCOCCN(C)C(=O)OC(C)(C)C)CCOCCN(C)C(=O)OC(C)(C)C]

L11-4 (406 mg, 0.62 mmol), L11-5 (150 mg, 0.80 mmol), and triphenylphosphine (241 mg, 0.92 mmol) were dissolved in tetrahydrofuran (10 mL) under nitrogen protection and were stirred at 0° C. for 10 min. Then, the solution of DIAD (250 mg, 1.24 mmol) in tetrahydrofuran solution (2 mL) was added dropwise. The reaction solution was warmed up to 80° C. and stirred for 12 hours. After the reaction completed, the solvent was removed by rotary evaporation and purification by silica gel column chromatography (PE:EA=1:1) was obtained to give the colorless oil L11-6 (190 mg).

### Step Three: Synthesis of Linker 11

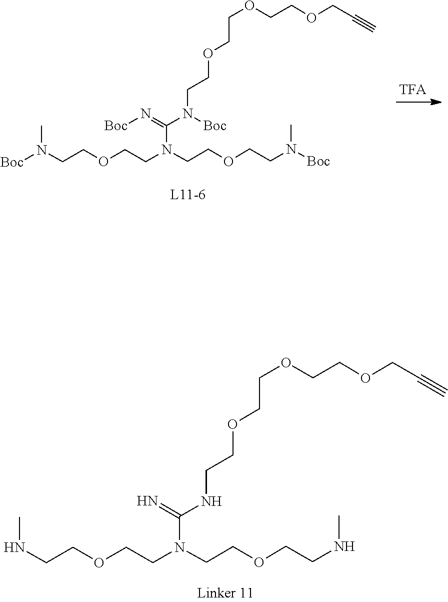

L11-6 (85 mg, 0.1 mmol) was placed in TFA (5 ml) and stirred at room temperature for 3 h. After the reaction completed, concentration was performed to give the Linker 11 that was directly used in the next step. LCMS[M+1]⁺: 432.41

### Example 15: Synthesis of Conjugate Intermediate C-Inter-1

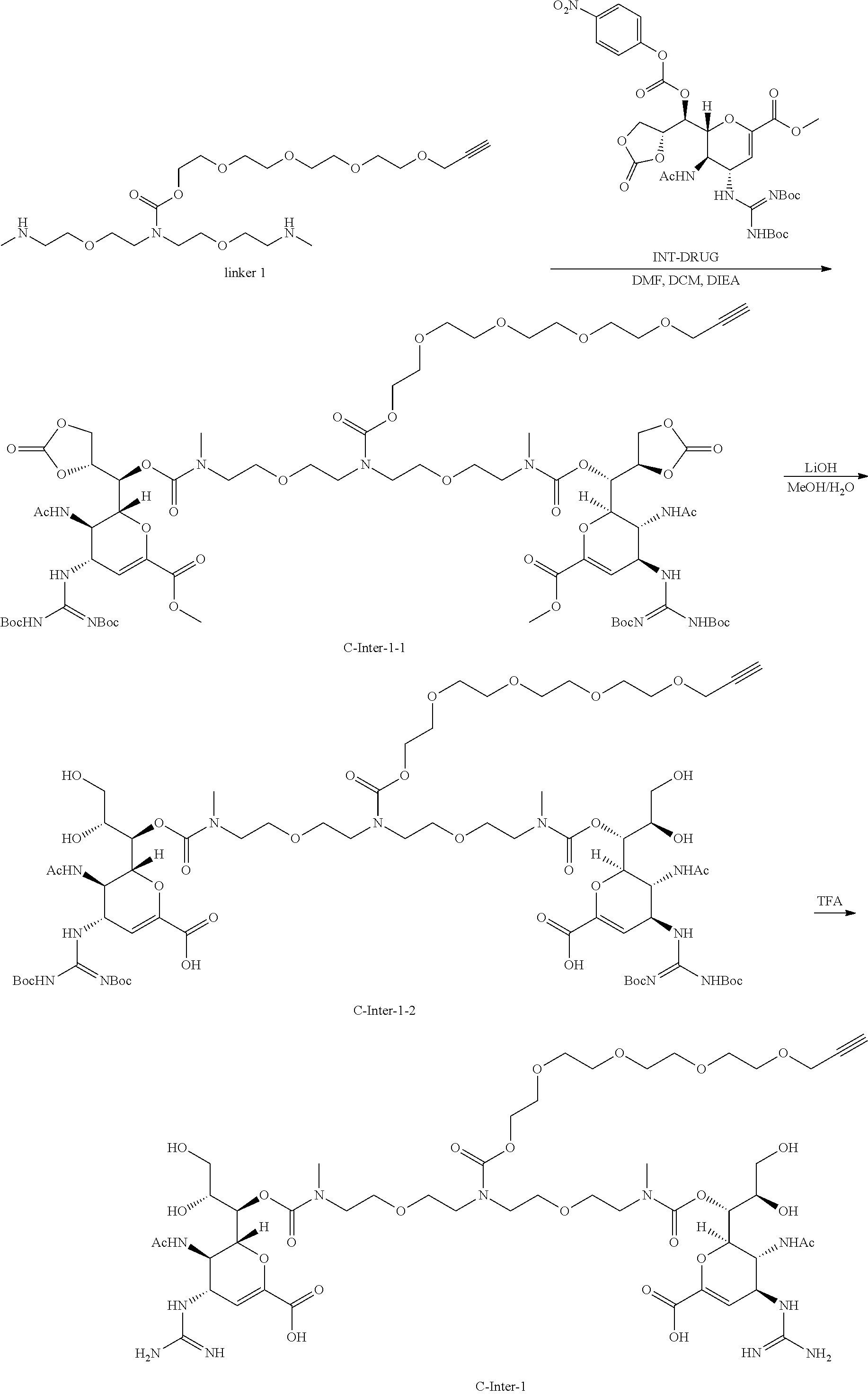

### Step One: Synthesis of C-Inter-1-1

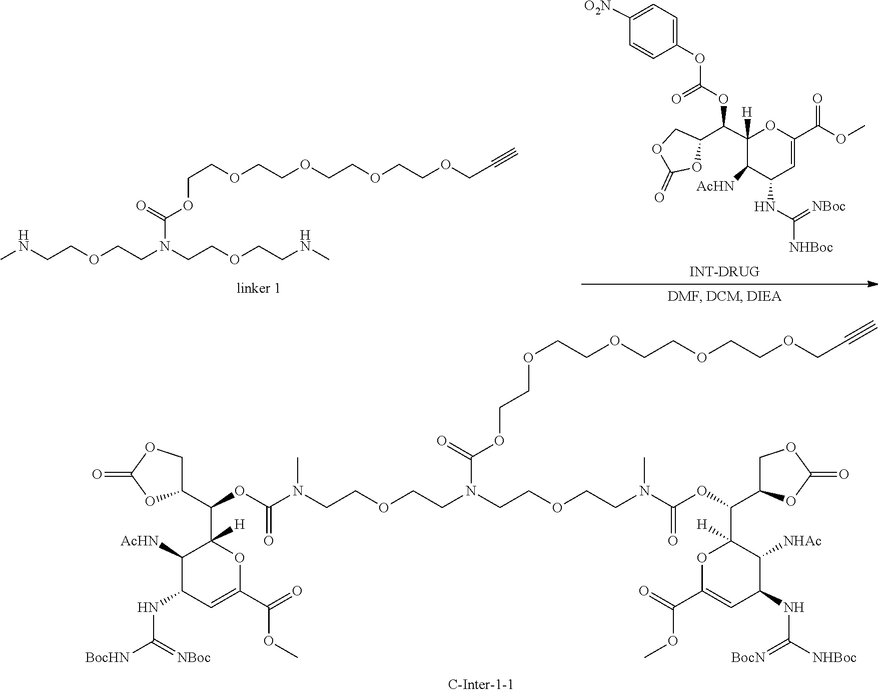

Linker 1 (65 mg, 0.13 mmol) was added to the reaction flask followed by addition of DMF (10 mL) and DIEA (2 mL), which were stirred at room temperature. The solution of INT-DRUG (200 mg, 0.27 mmol) dissolved in dichloromethane (5 mL) was added dropwise to the reaction solution that was stirred at room temperature overnight. After the reaction completed, the solvent was removed by rotary evaporation to give the crude and purification by reverse column (water/acetonitrile) and freeze-drying were performed to give the C-Inter-1-1 (216 mg).

### Step Two: Synthesis of C-Inter-1-2

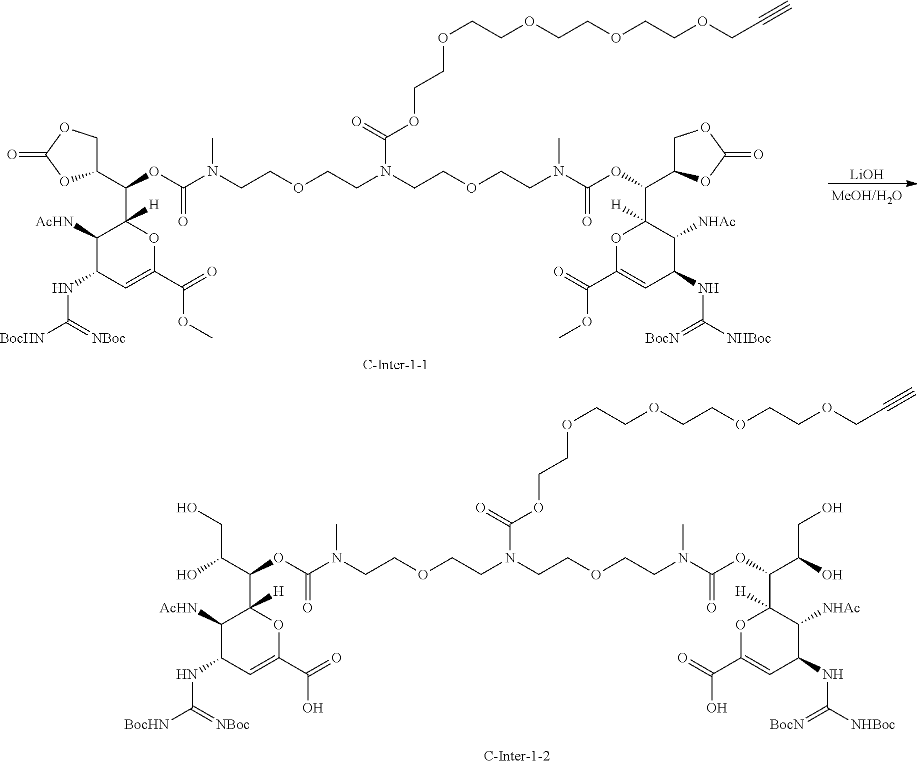

Compound C-Inter-1-1 (216 mg, 0.13 mmol) was dissolved in methanol (2 mL) followed by addition of aqueous lithium hydroxide (0.65 mmol, 2 mL), which were stirred at room temperature for 1 hour. After the reaction completed, the solvent was removed by rotary evaporation to give the crude compound C-Inter-1-2, which was used directly in the next step.

### Step Three: Synthesis of C-Inter-1

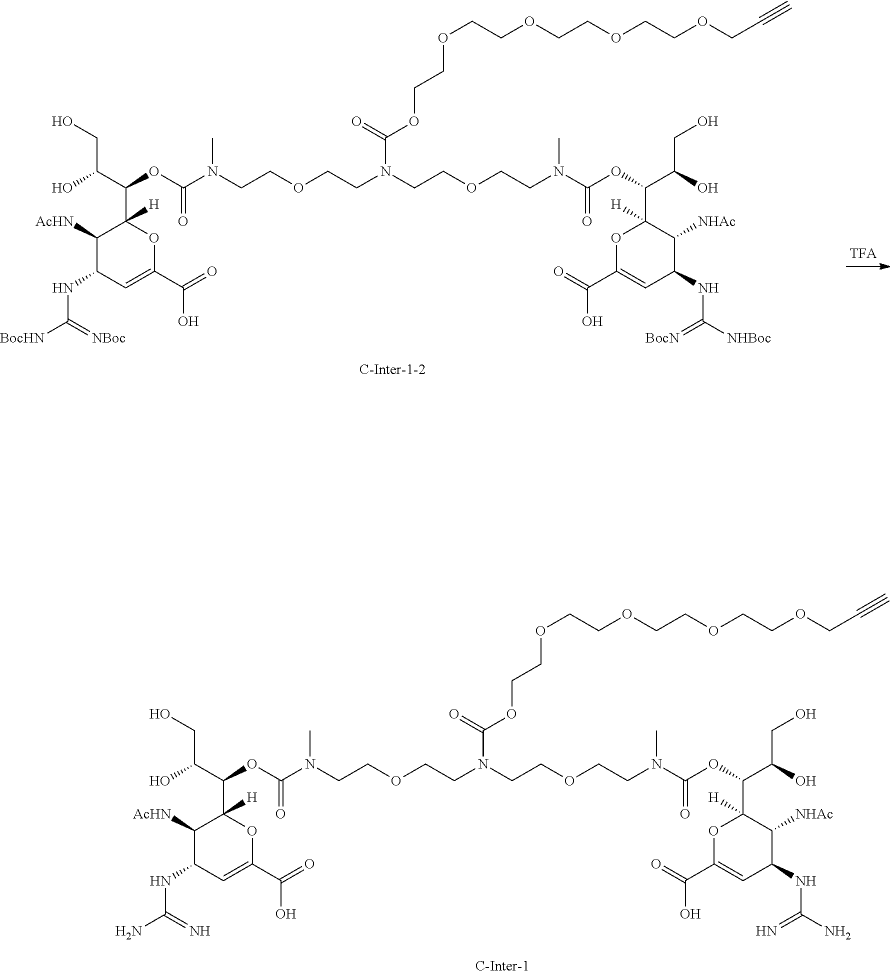

Compounds C-Inter-1-2 were dissolved in trifluoroacetic acid, and stirred at room temperature for 10 min. The solvent was removed by rotary evaporation to give the crude C-Inter-1. Purification by reverse column chromatography (0.1% TFA aqueous solution/0.1% TFA acetonitrile) and freeze dried were performed to give the C-Inter-1 (47 mg). LCMS: [M+1]+ found 1166.53.

### Example 16: Synthesis of Conjugate Intermediate C-Inter-2

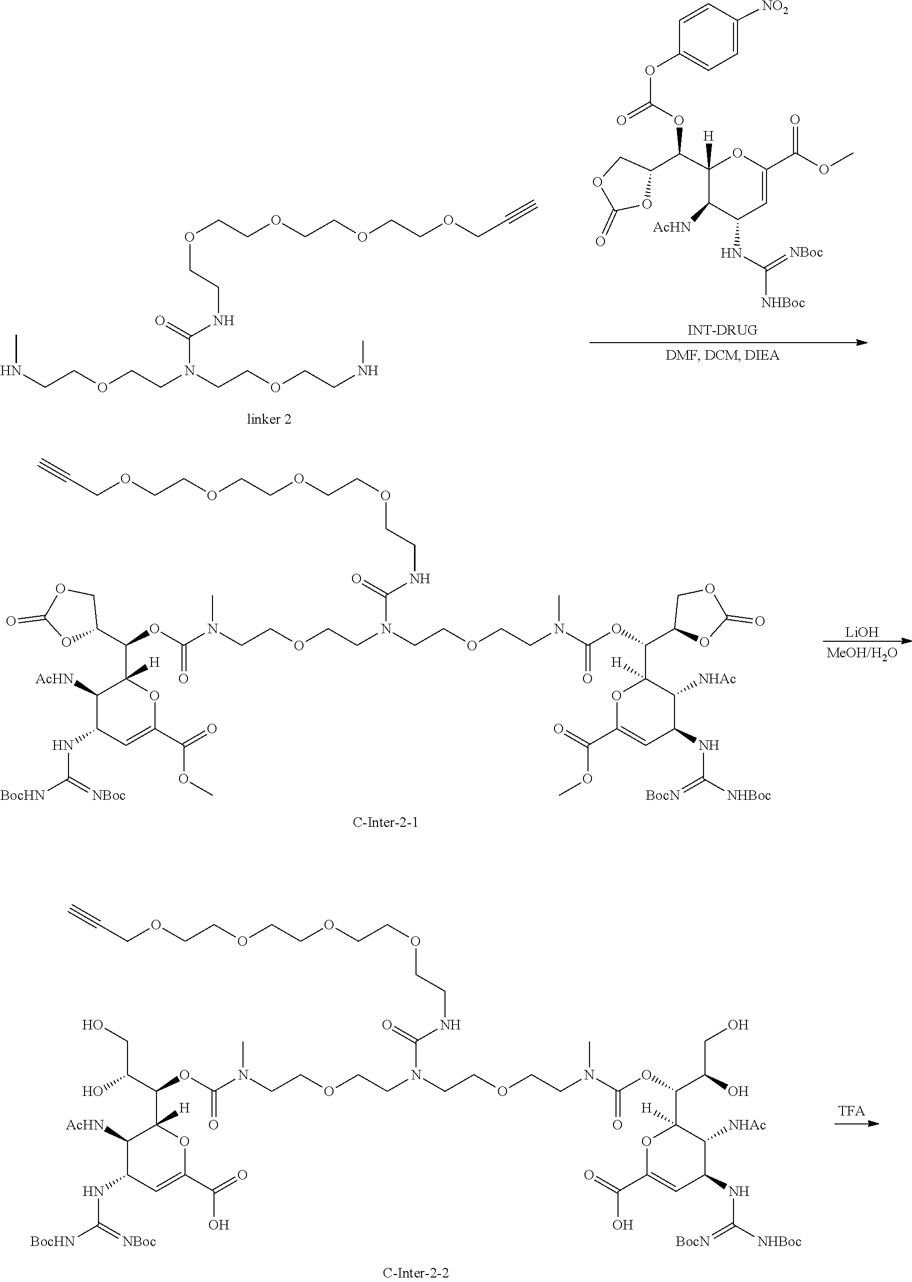

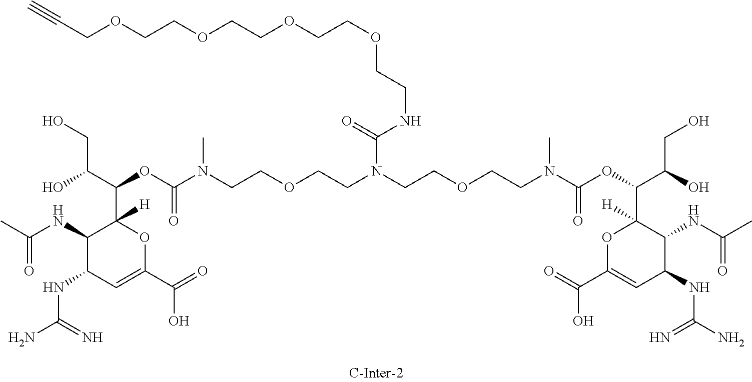 [SMILES: C#CCOCCOCCOCCOCCNC(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO]

### Step One: Synthesis of C-Inter-2-1

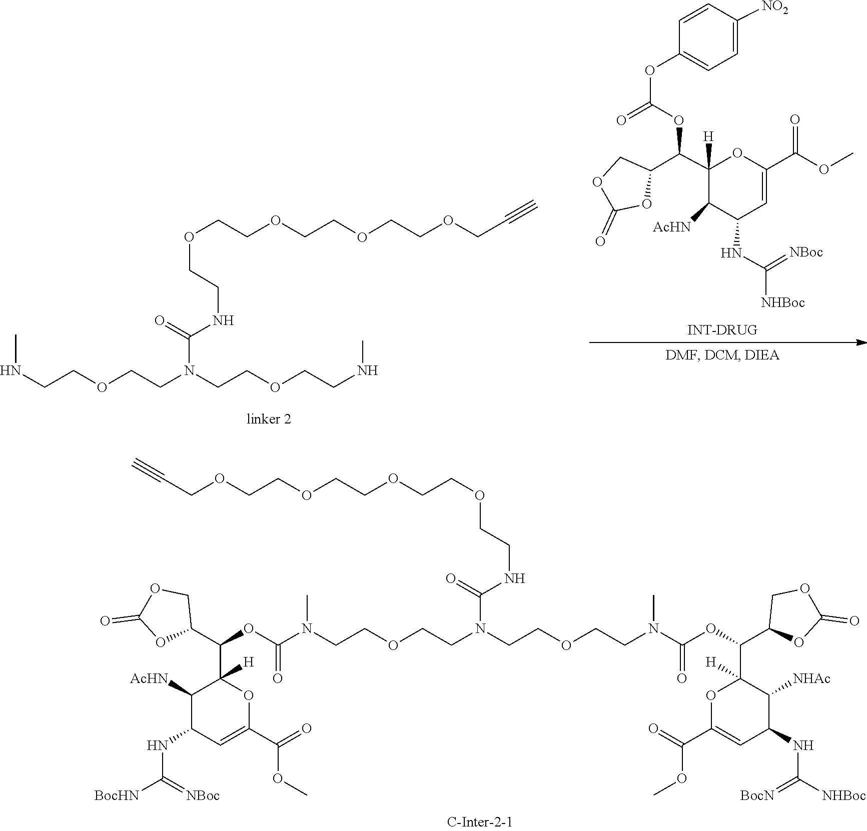

Linker 2 (100 mg, 0.20 mmol) dissolved in DMF (1 ml) was added with DIEA (0.1 g, 1.23 mmol), to which the INT-DRUG (300 mg, 0.41 mmol) dissolved in DCM (2 ml) was added dropwise. The mixture was stirred at room temperature for 16 h, and then concentrated under reduced pressure followed by the reversed-column chromatography to give C-Inter-2-1 (230 mg).

### Step Two: Synthesis of C-Inter-2-2

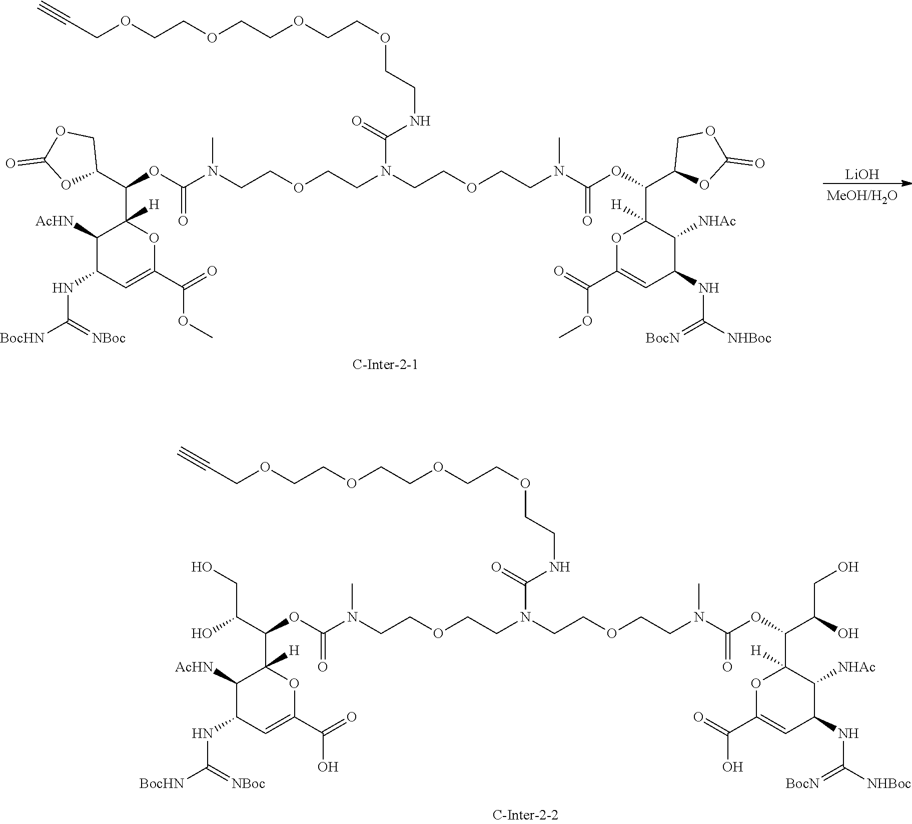

Compound C-Inter-2-1 (230 mg, 0.14 mmol) was dissolved in methanol (5 mL) followed by addition of aqueous lithium hydroxide (30 mg, 1 ml), which were stirred at room temperature for 1 hour. Then, concentration was directly performed to give the crude compound C-Inter-1-2, which was used directly in the next step.

### Step Three: Synthesis of C-Inter-2

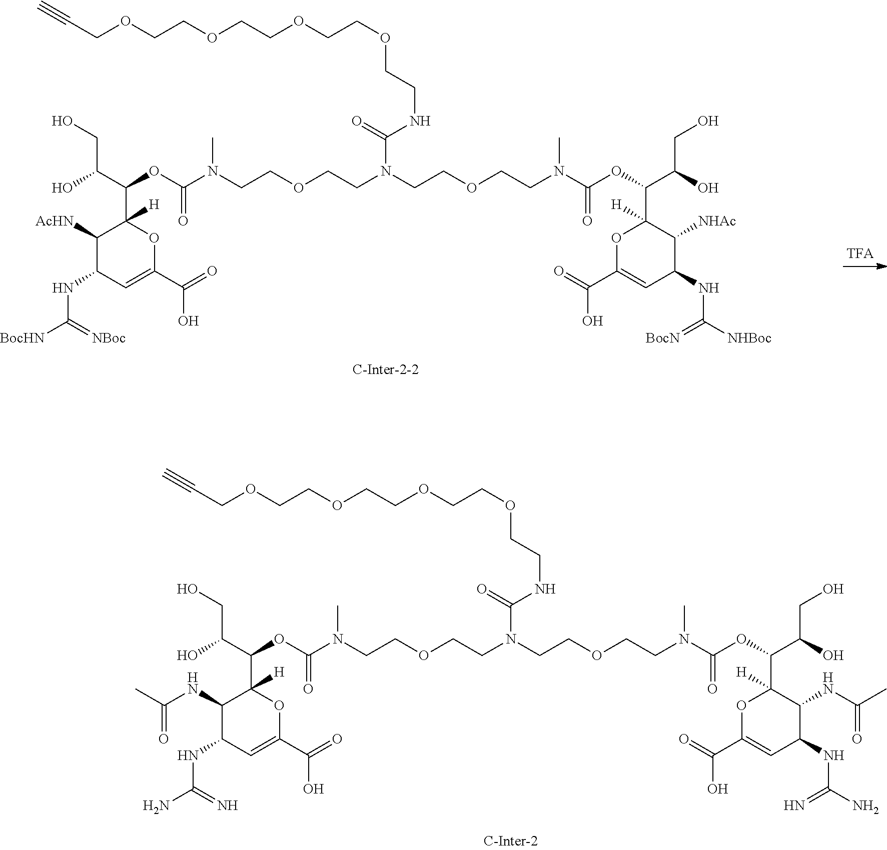

The crude C-Inter-2-2 (300 mg, calculated as 0.14 mmol) was dissolved in TFA (5 ml), and stirred at room temperature for 5 min. The concentration under reduced pressure, and purification by reversed-phase preparative HPLC were performed to give C-Inter-2 (72 mg). LCMS[M+1]+:1194.00.

### Example 17: Synthesis of Conjugate Intermediate C-Inter-3

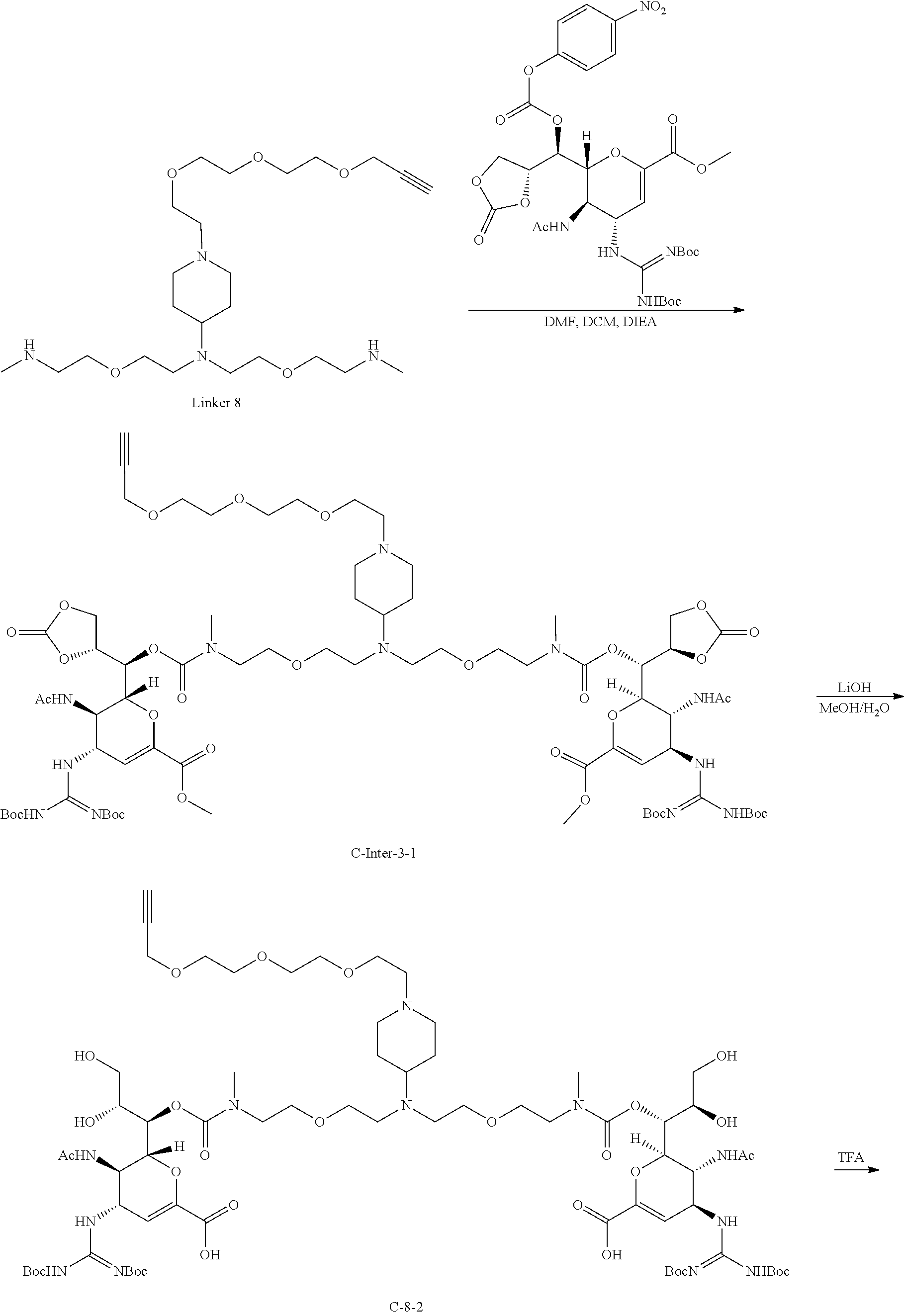

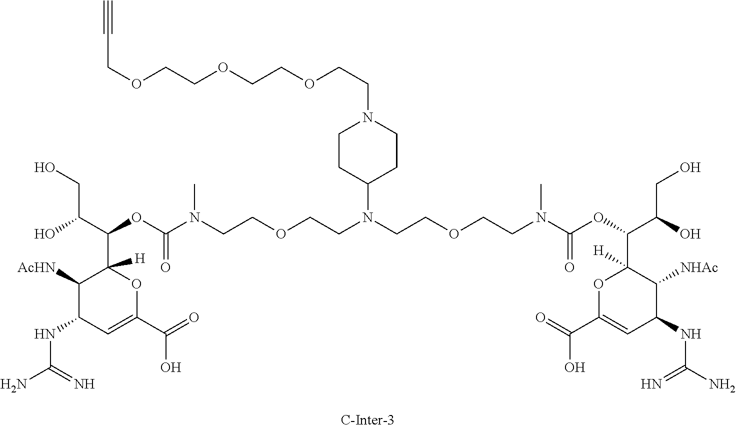

### Step One: Synthesis of C-Inter-3-1

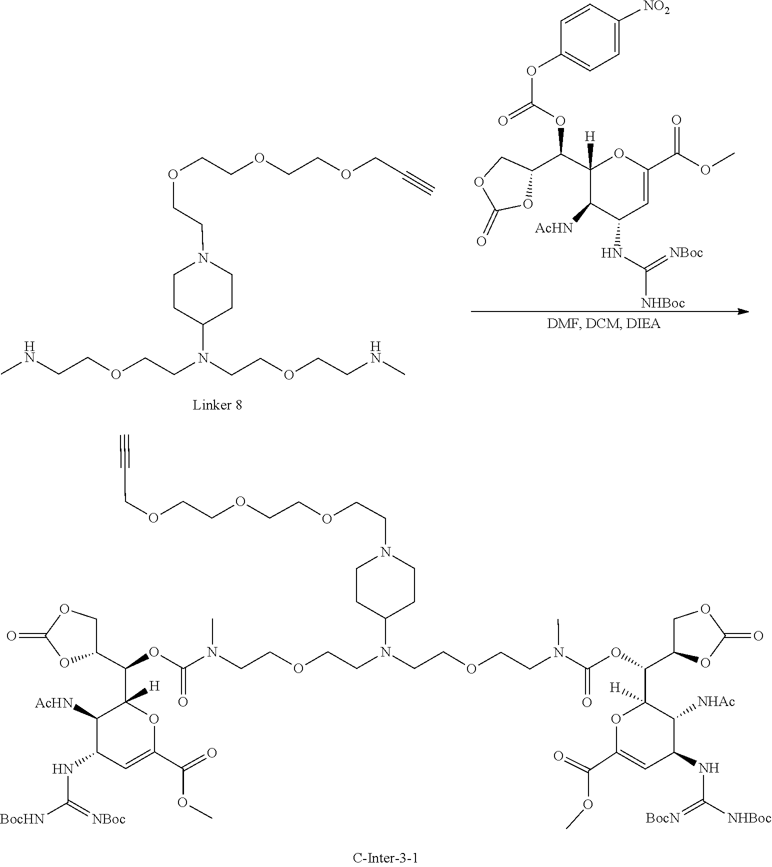

The crude Linker 8 (80 mg, calculated as 0.15 mmol) dissolved in DMF (1 ml) was added with DIEA (0.6 g, 4.5 mmol), to which the INT-DRUG (210 mg, 0.3 mmol) dissolved in DCM (2 ml) was added dropwise. The mixture was stirred at room temperature for 16 h. Subsequently, concentration under reduced pressure was performed to give the crude C-Inter-3-1 (300 mg), which was directly used in the next step.

### Step Two: Synthesis of C-Inter-3-2

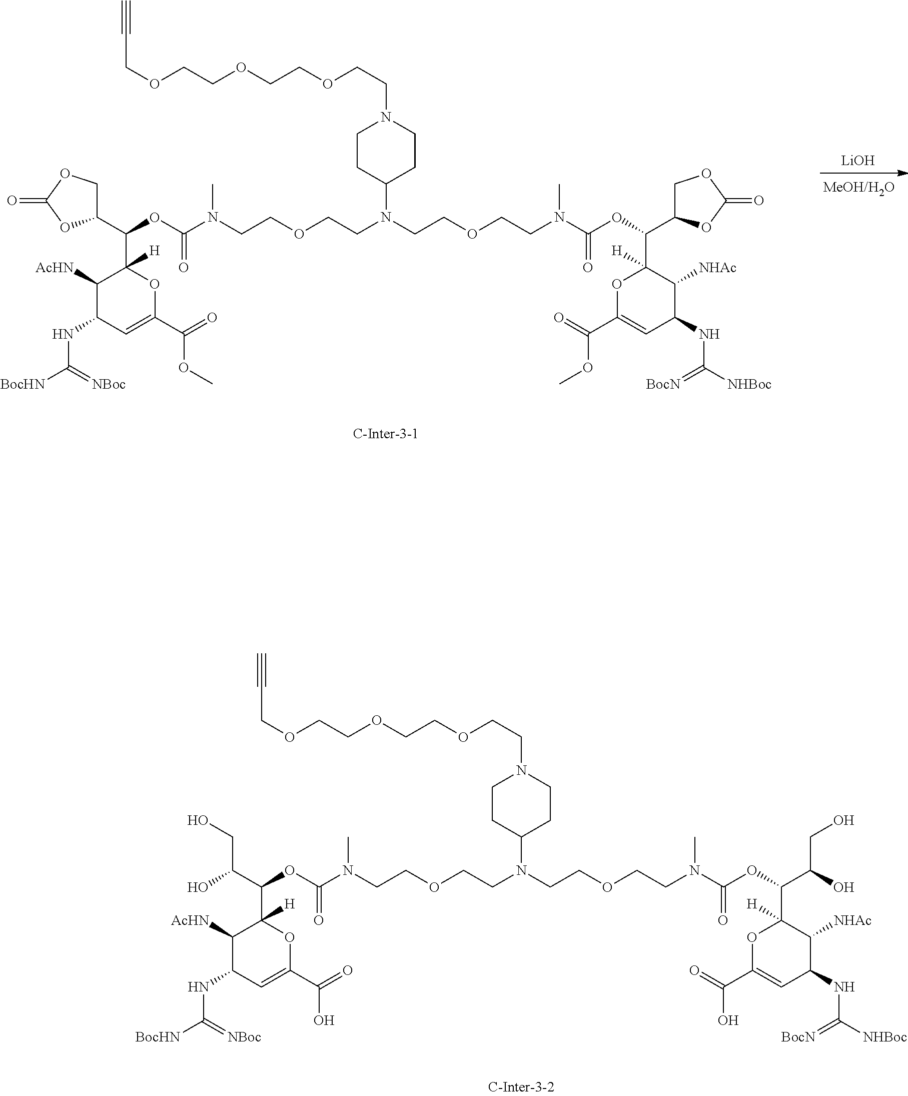

Crude C-Inter-3-1 (300 mg, calculated as 0.15 mmol) was dissolved in methanol (5 mL) followed by addition of aqueous lithium hydroxide (30 mg, 1 ml), which were stirred at room temperature for 1 hour. Then, concentration under reduced pressure was performed to give the crude C-Inter-3-2 (350 mg), which was used directly in the next step.

### Step Three: Synthesis of C-Inter-3

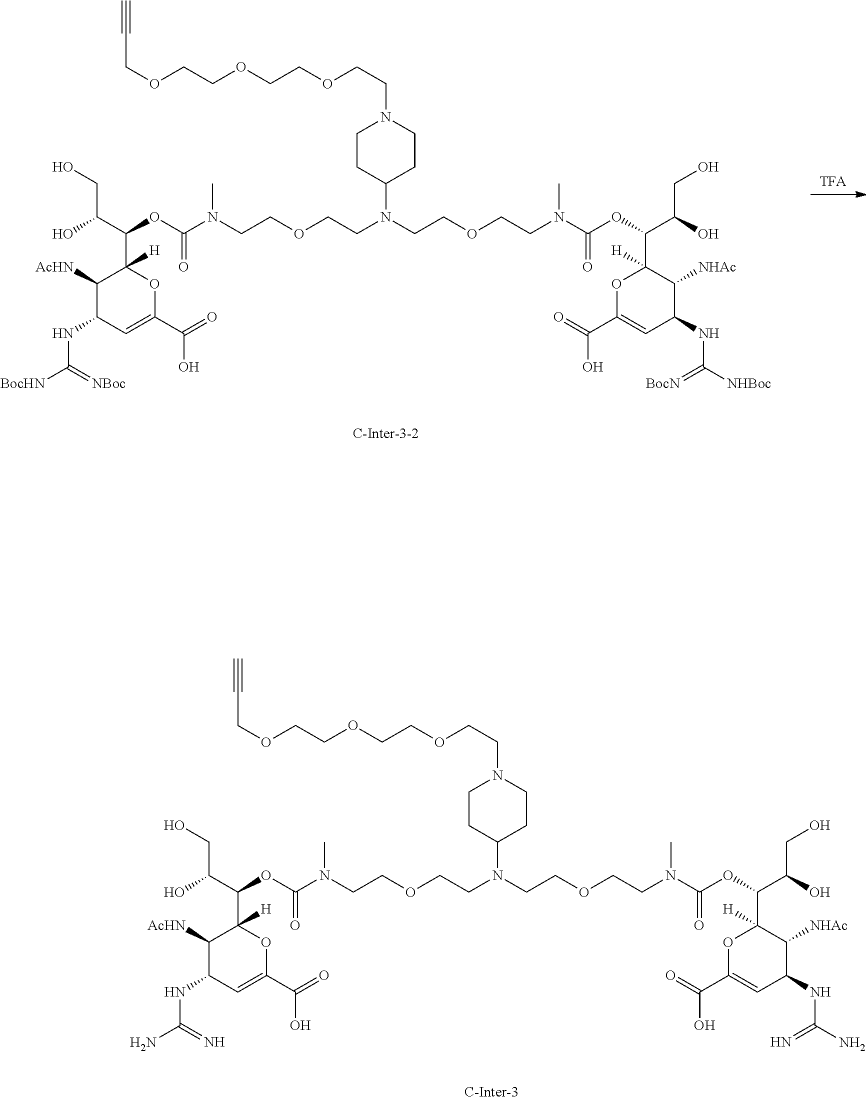

The crude C-Inter-3-2 (350 mg, calculated as 0.15 mmol) was dissolved in TFA (5 ml), and stirred at room temperature for 5 min. Then, concentration under reduced pressure and reverse preparative HPLC were performed to give the C-Inter-3 (20 mg). LCMS[M+1]+:1189.50.

### Example 18: Synthesis of Conjugate Intermediate C-Inter-4

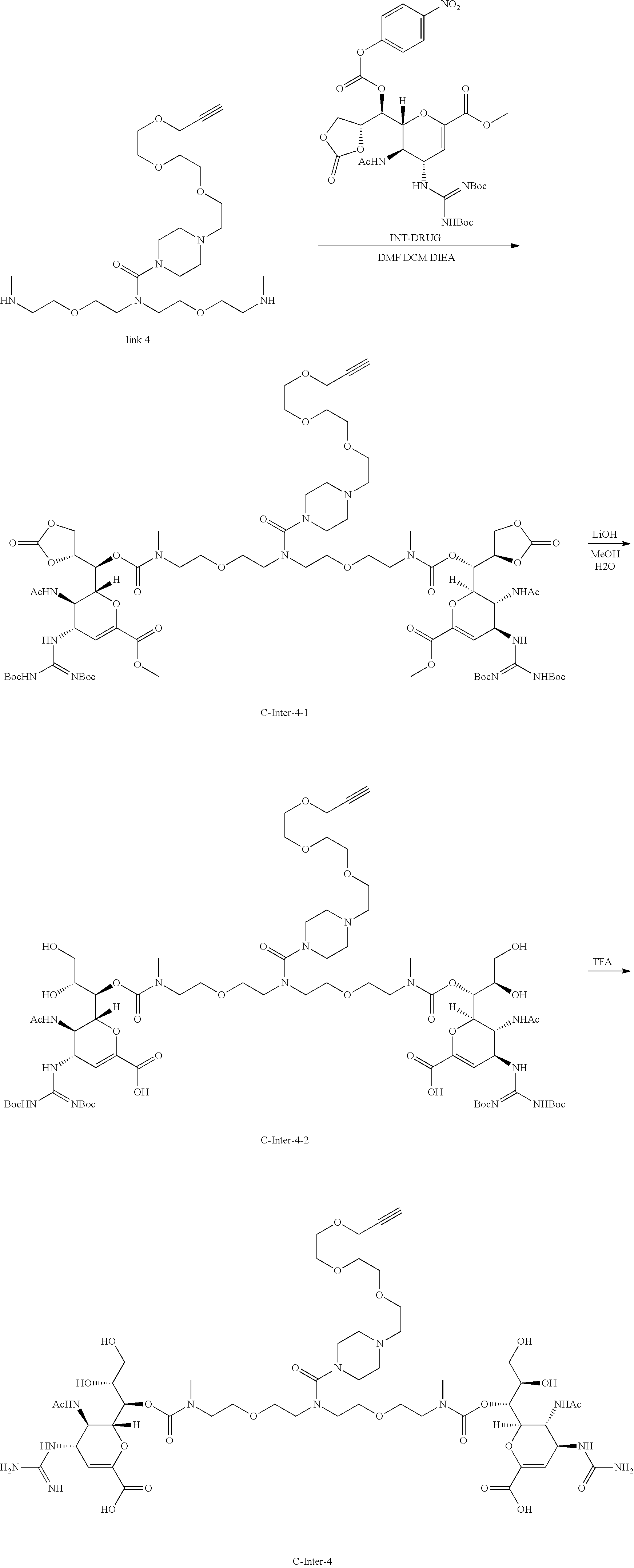

### Step One: Synthesis of C-Inter-4-1

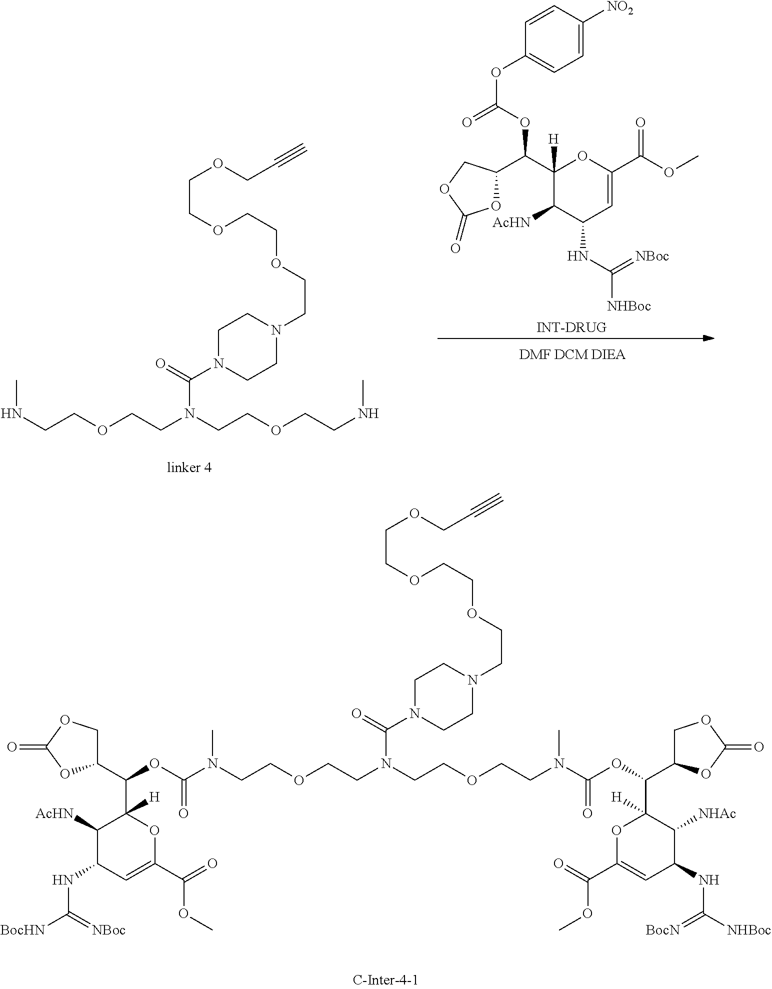

Linker 4 (70 mg, 0.14 mmol) was added to the reaction flask followed by addition of DMF (10 mL) and DIEA (2 mL), which were stirred at room temperature. The solution of INT-DRUG (180 mg, 0.24 mmol) dissolved in dichloromethane (5 mL) was added dropwise to the reaction solution and stirred at room temperature overnight. After the reaction completed, the solvent was removed by rotary evaporation to give the crude compound, and the purification by reverse column (water/acetonitrile) and freeze-drying were performed to give the product C-Inter-4-1 (210 mg).

### Step Two: Synthesis of C-Inter-4-2

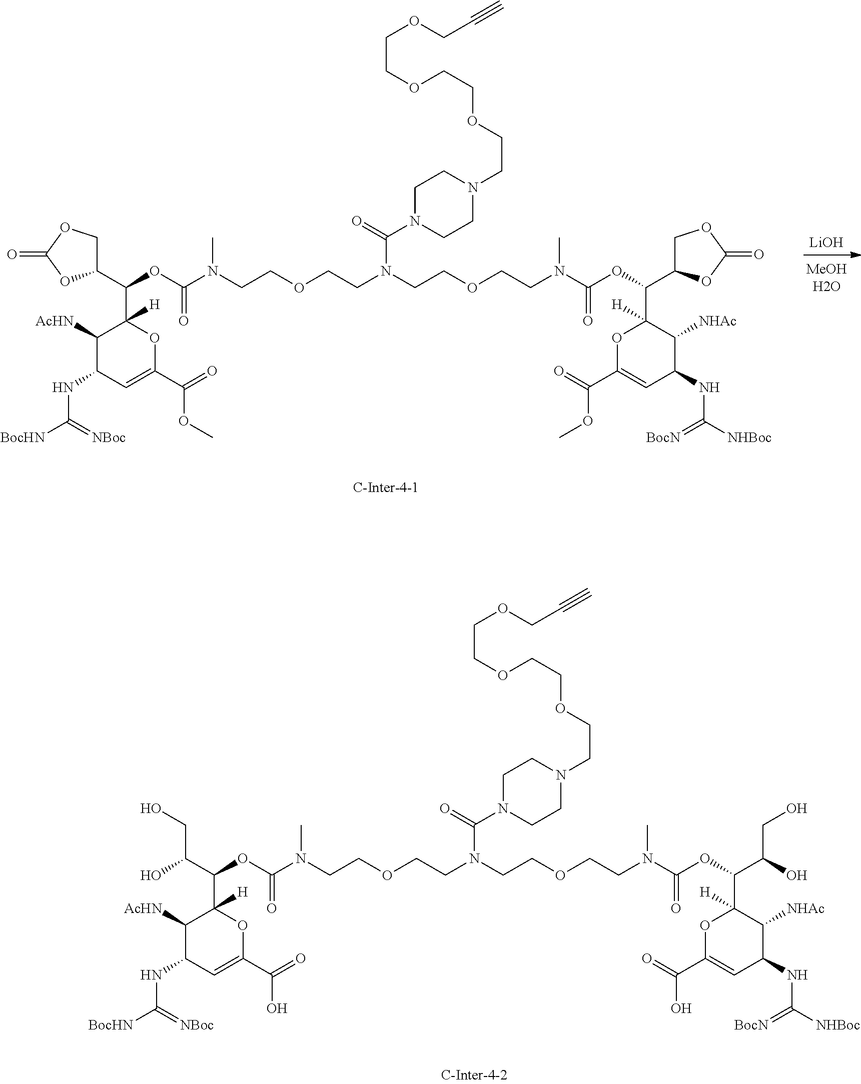

Compound C-Inter-4-1 (210 mg, 0.12 mmol) dissolved in methanol (2 mL) was added with aqueous lithium hydroxide (0.65 mmol, 2 mL), which was stirred at room temperature for 1 hour. After the reaction completed, the solvent was removed by rotary evaporation to give the crude compound C-Inter-4-2, which was used directly in the next step.

### Step Three: Synthesis of C-Inter-4

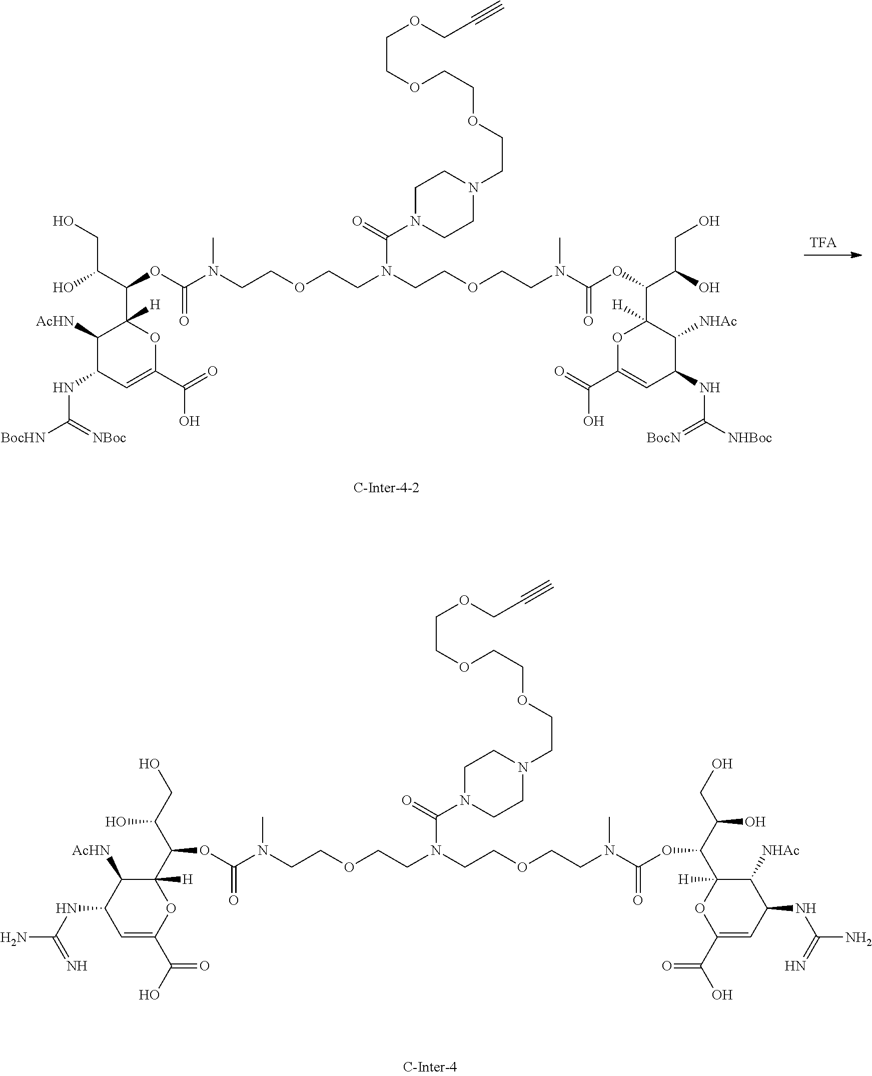

The crude C-Inter-4-2 (350 mg, calculated as 0.15 mmol) was dissolved in TFA (5 ml), and stirred at room temperature for 5 min. Then concentration under reduced pressure and preparative HPLC (P-HPLC) chromatography were performed to give the C-Inter-4 (20 mg). LCMS[M+1]+:1189.50.

### Example 19: Synthesis of Conjugate Intermediate C-Inter-5

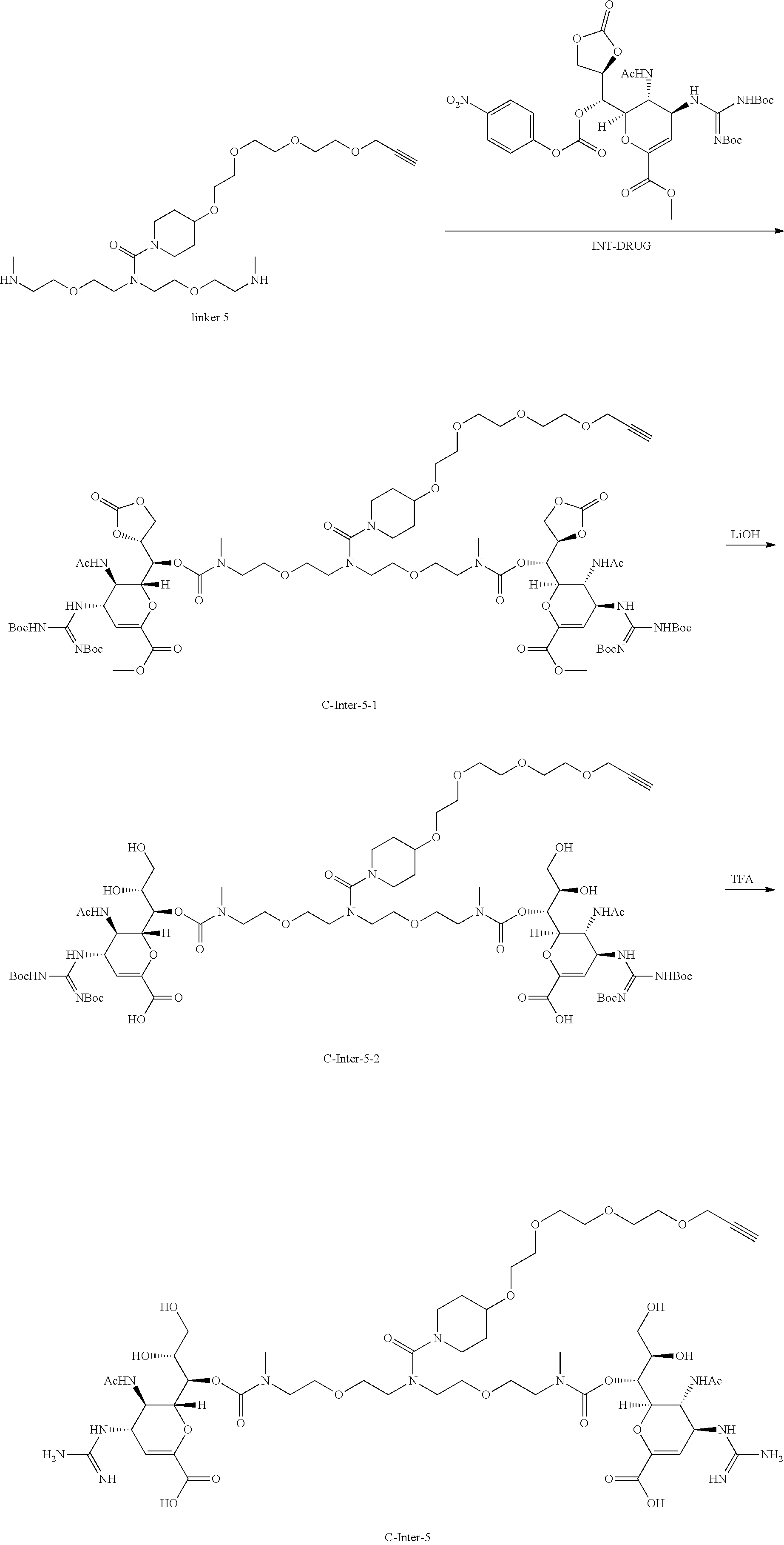

### Step One: Synthesis of C-Inter-5-1

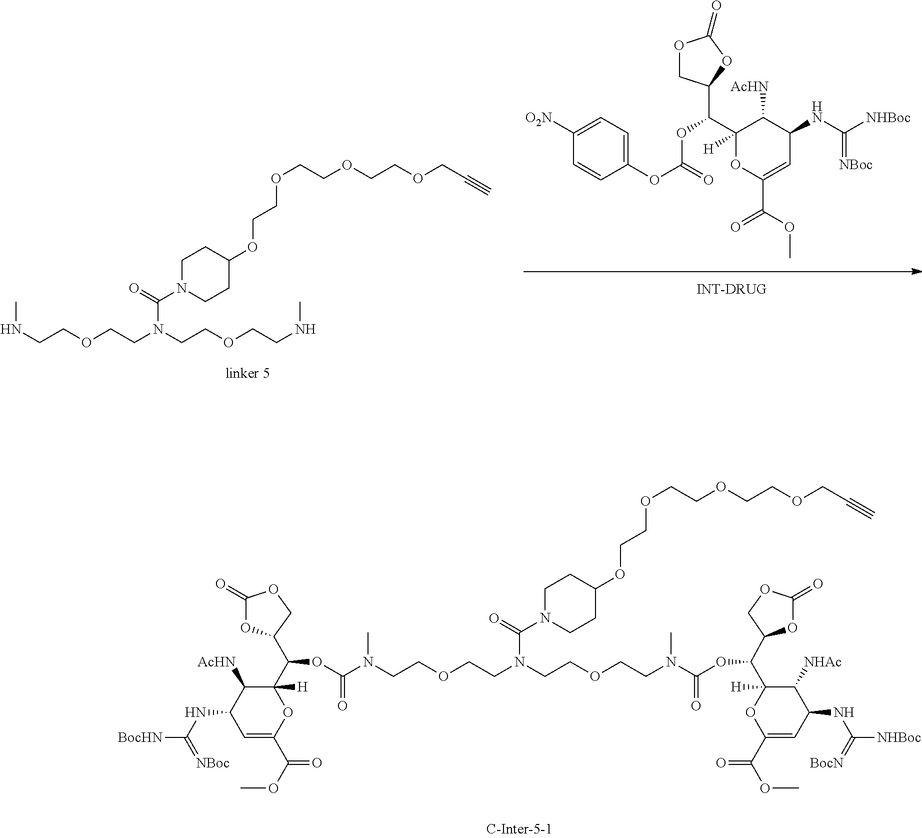

The crude Linker 5 (86 mg) dissolved in DMF (1 ml) was added with DIEA (0.5 mL), to which INT-DRUG (230 mg, 0.32 mmol) dissolved in DCM (2 ml) was added dropwise. The mixture was stirred for 16 h at room temperature, and then concentrated under reduced pressure to obtain the crude C-Inter-5-1 crude. Purification by reverse column chromatography was performed to give the target product (170 mg).

### Step Two: Synthesis of C-Inter-5-2

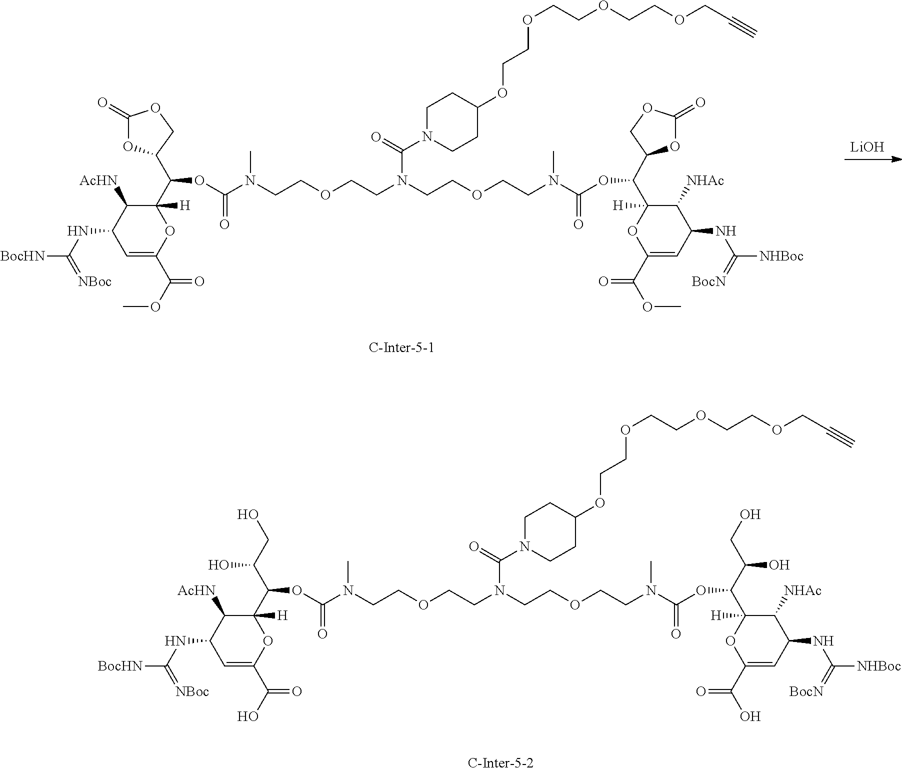

C-Inter-5-1 (170 mg) dissolved in methanol (5 ml) was added with aqueous lithium hydroxide (21 mg, 1 ml), stirred at room temperature for 1 h and concentrated under reduced pressure to give the crude C-Inter-5-2, which was used directly in the next step.

### Step Three: Synthesis of C-Inter-5

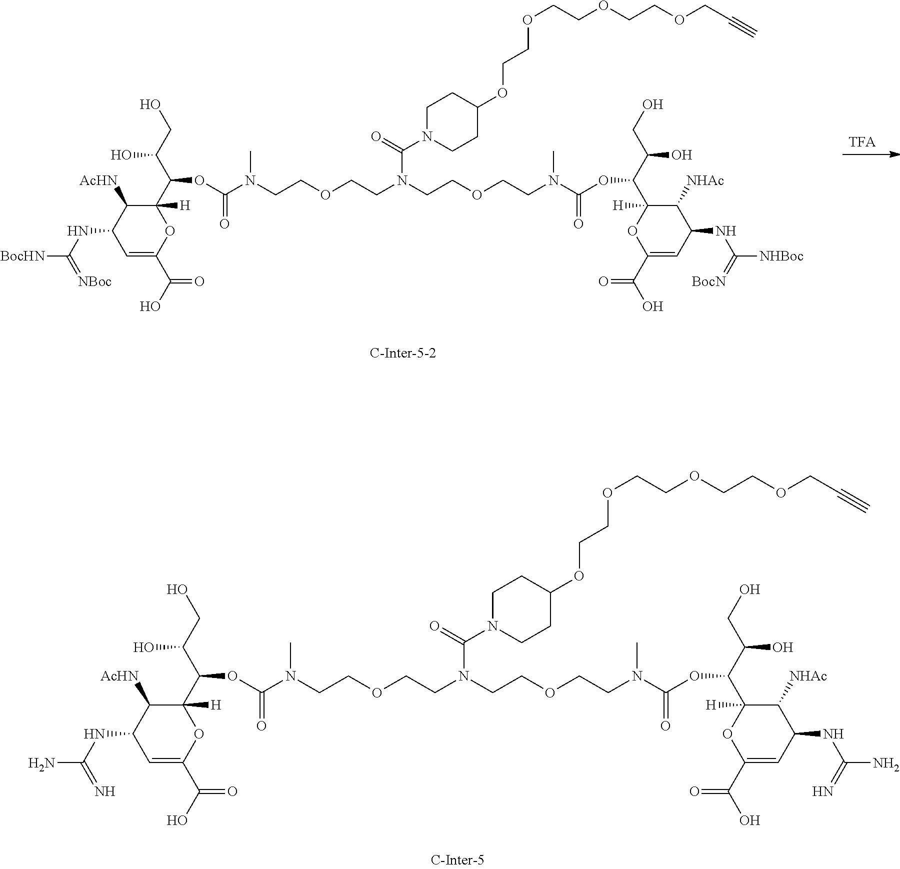

The crude C-Inter-5-2 (350 mg) was dissolved in TFA (5 ml), stirred at room temperature for 5 min, concentrated under reduced pressure and subjected to preparative HPLC (P-HPLC) chromatography to give the C-Inter-5 (72 mg). LCMS [M/2+1]+: 617.45.

### Example 20: Synthesis of C-Inter-6

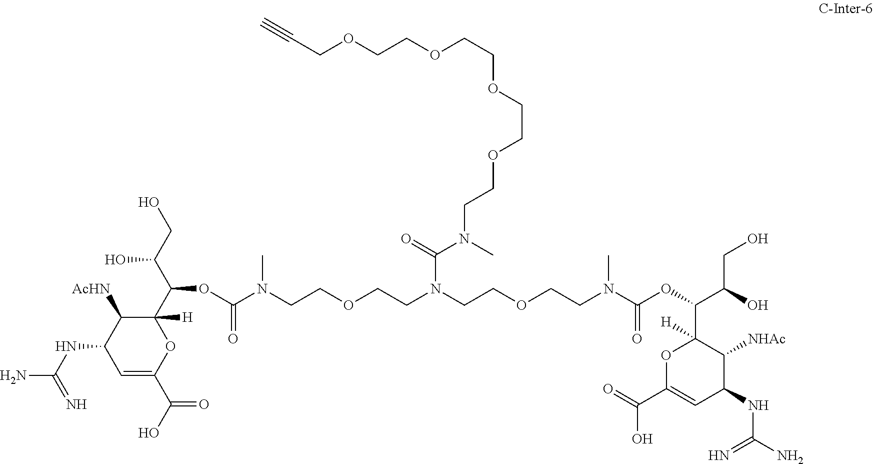 [SMILES: C#CCOCCOCCOCCOCCN(C)C(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO]

### Step One: Synthesis of C-Inter-6-2

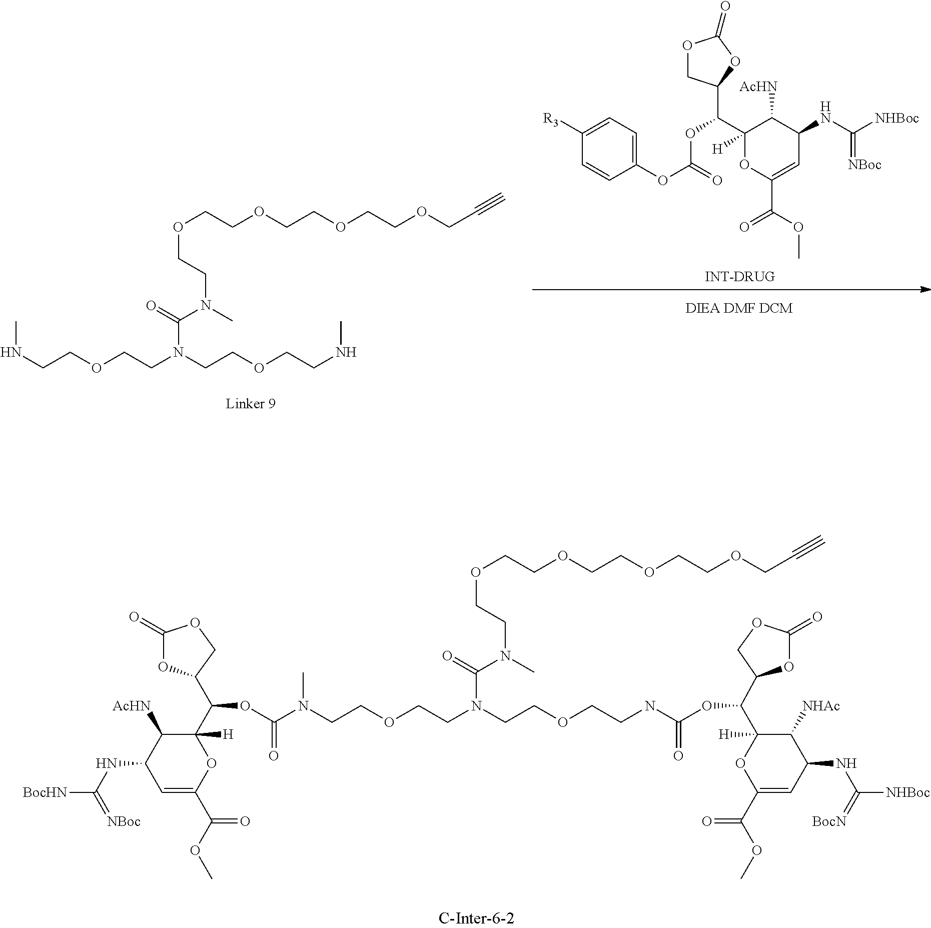

Compound Linker 9 (80 mg, 0.15 mmol) was added to the reaction flask followed by DMF (4 mL) and DIEA (2 mL), which was then stirred at room temperature. INT-DRUG (220 mg, 0.3 mmol) dissolved in dichloromethane (2 mL) was added dropwise to the reaction solution that was then stirred at room temperature overnight. After the reaction completed, the solvent was removed by rotary evaporation to give the crude compound, and purification by reverse column (water/acetonitrile) and freeze-drying were performed to give the product C-Inter-6-2 (190 mg).

### Step Two: Synthesis of C-Inter-6-3

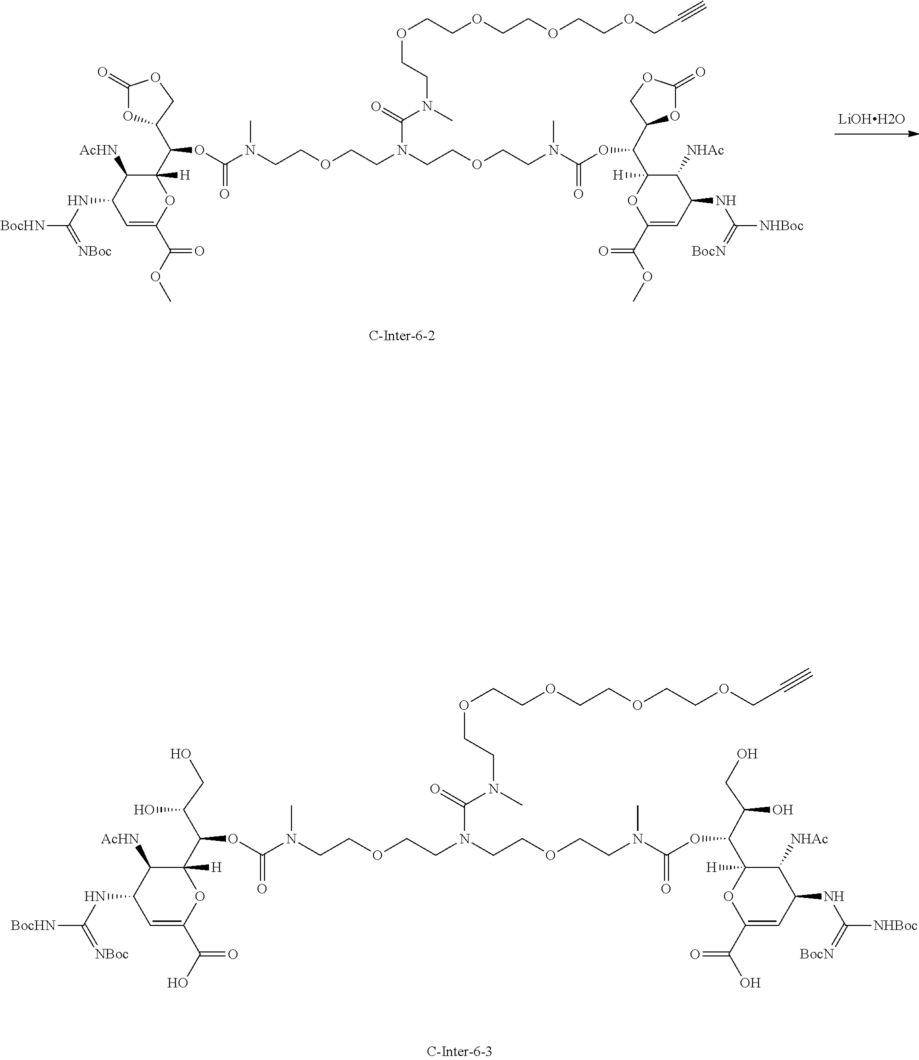

Compound C-Inter-6-2 (110 mg, 0.065 mmol) dissolved in methanol (5 mL) was added with the aqueous solution (1.5 mL) of lithium hydroxide monohydrate (12 mg, 0.28 mmol), which was then stirred for 4 hours at room temperature. After the reaction completed, the solvent was removed by rotary evaporation to give the crude compound C-Inter-6-3 that was directly used in the next step.

### Step Three: Synthesis of C-Inter-6-3

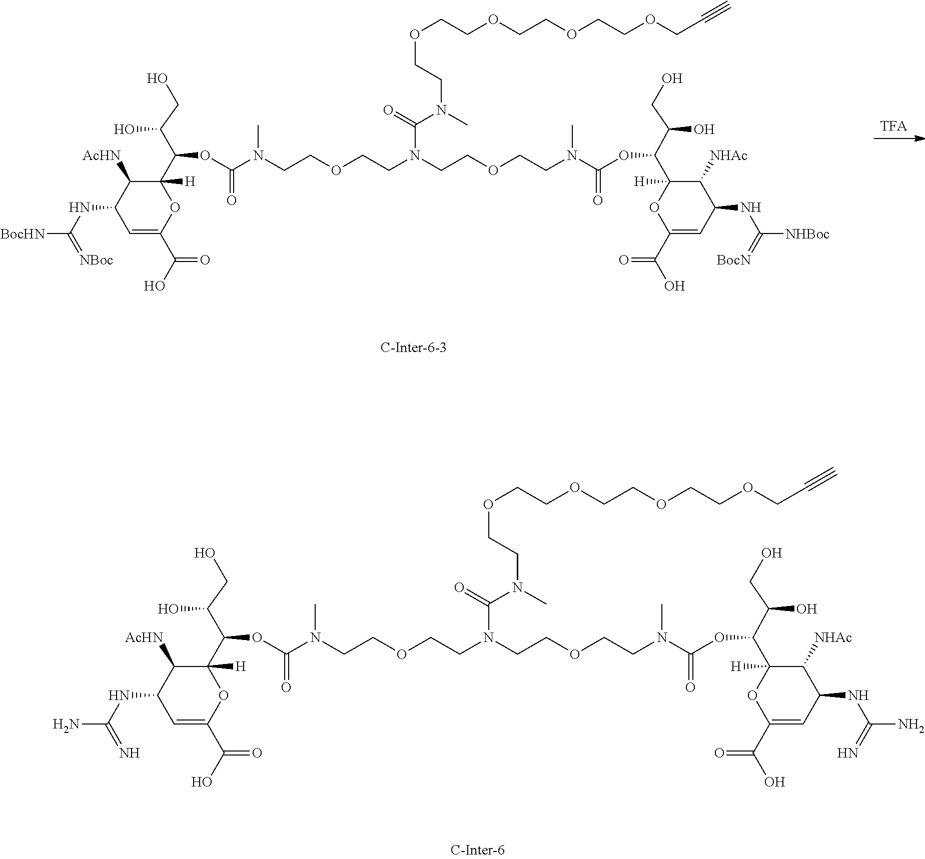

Compound C-Inter-6-3 was dissolved in trifluoroacetic acid (3 mL) and stirred at room temperature for 15 min. Then, the solvent was removed by rotary evaporation to give the crude compound. The crude compound was purified by reverse column chromatography (0.1% TFA aqueous solution/0.1% TFA acetonitrile) and freeze dried to give C-Inter-6 (53 mg). LCMS: [M+1]⁺ found 1207.2.

### Example 21: Synthesis of C-Inter-7

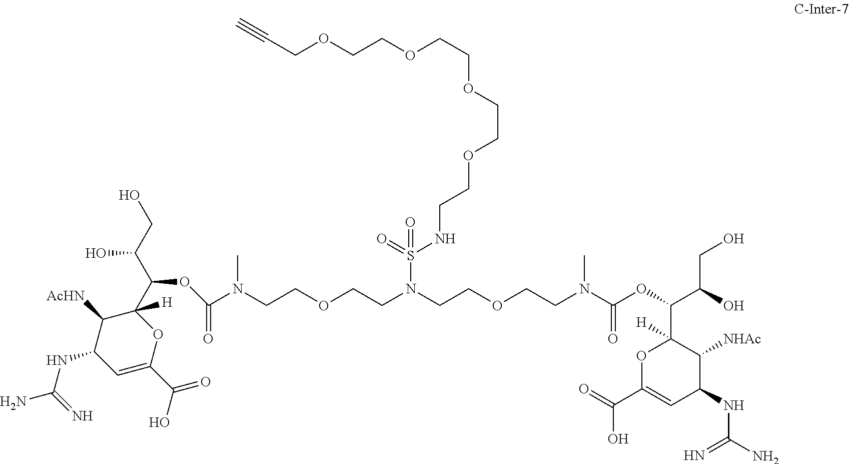 [SMILES: C#CCOCCOCCOCCOCCNS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO]

### Step One: Synthesis of C-Inter-7-1

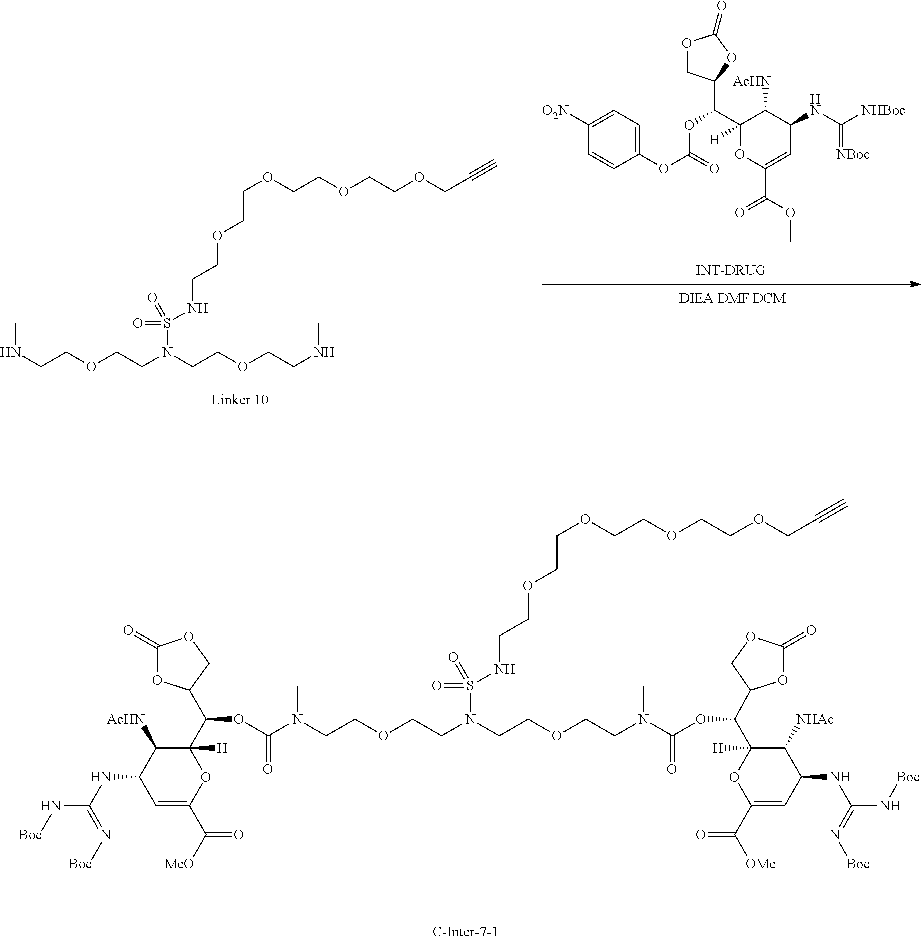

Linker 10 (calculated as 0.15 mmol) dissolved in DMF (2 ml) was added with DIEA (0.6 g, 4.5 mmol), to which INT-DRUG (210 mg, 0.3 mmol) dissolved in DCM (2 ml) was added dropwise. The mixture was stirred at room temperature for 16 h, then concentrated under reduced pressure to obtain the crude product. The crude product was purified by reversed-phase column chromatography (acetonitrile/water) and freeze dried to give the white solid C-Inter-7-1 (180 mg). LCMS[M12+1]+: 855.5.

### Step Two: Synthesis of C-Inter-7-2

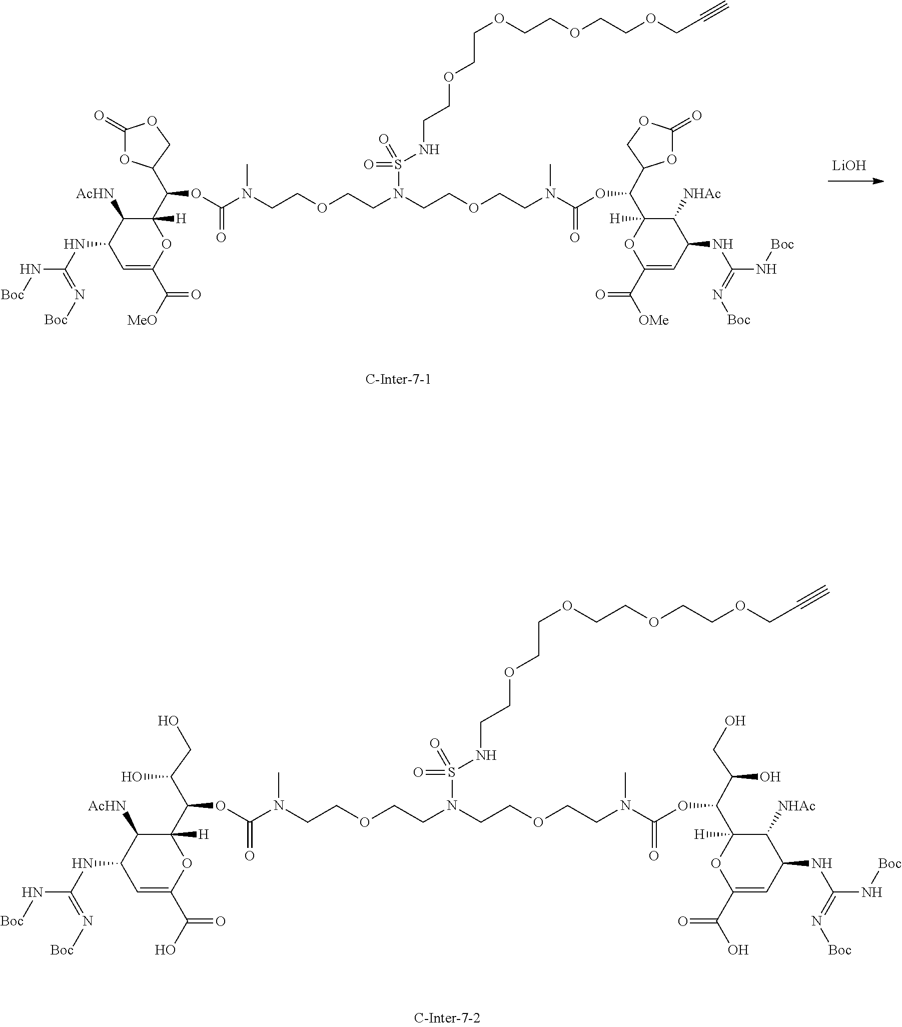

C-Inter-7-1 (180 mg, 0.11 mmol) dissolved in methanol (5 ml) was added with aqueous lithium hydroxide (0.55 mmol, 1 ml), which was stirred at room temperature for 1 h. After addition of acetic acid (0.3 mL), rotary evaporation was performed to obtain the crude product C-Inter-7-2, which was directly used in the next step.

### Step Three: Synthesis of C-Inter-7

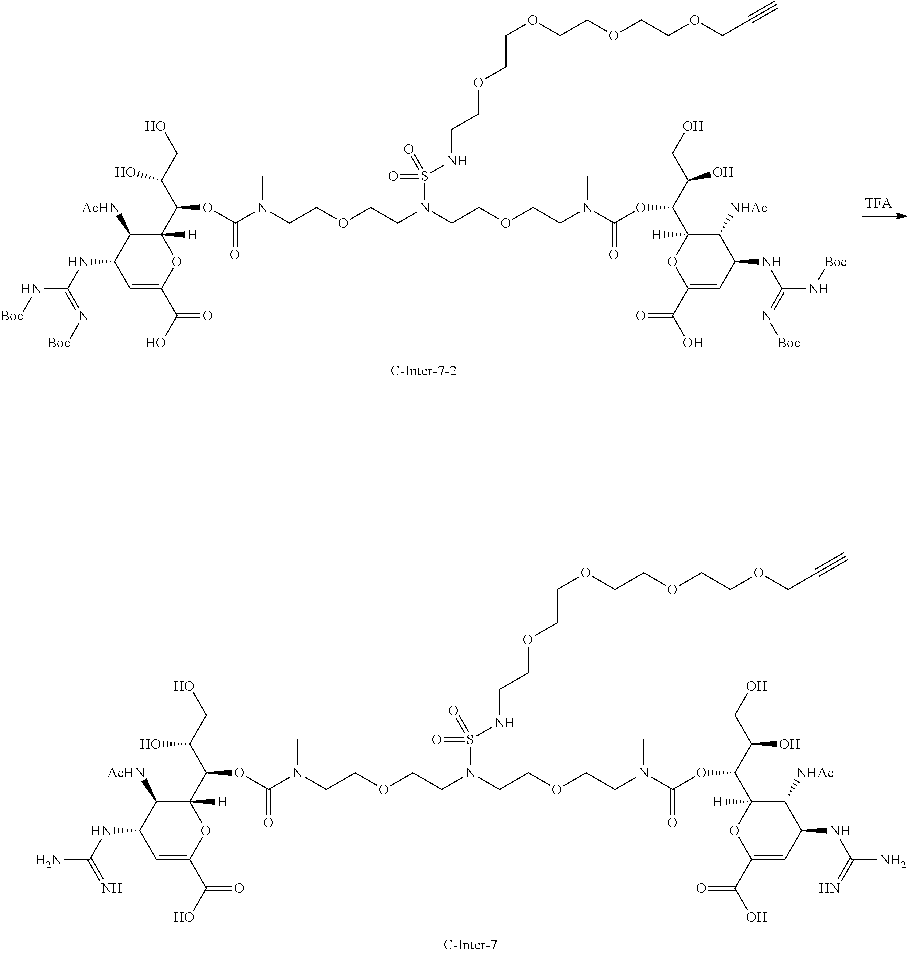 [SMILES: C#CCOCCOCCOCCOCCNS(=O)(=O)N(CCOCCN(C)C(=O)O[C@@H]([C@@H]1OC(C(=O)O)=C[C@H](N/C(=N/C(=O)OC(C)(C)C)NC(=O)OC(C)(C)C)[C@H]1NC(C)=O)[C@H](O)CO)CCOCCN(C)C(=O)O[C@@H]([C@@H]1OC(C(=O)O)=C[C@H](N/C(=N/C(=O)OC(C)(C)C)NC(=O)OC(C)(C)C)[C@H]1NC(C)=O)[C@H](O)CO ; C#CCOCCOCCOCCOCCNS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO]

The crude C-Inter-7-2 placed in trifluoroacetic acid (1 ml) was stirred at room temperature for 30 min. After the reaction completed, rotary evaporation was performed to remove the solvent and give the crude product. The crude product was purified by reverse column purification (acetonitrile/0.1% TFA aqueous solution) and freeze dried to give the C-Inter-7 (77 mg). LCMS [M+1]+:1229.5.

### Example 22: Synthesis of C-Inter-10

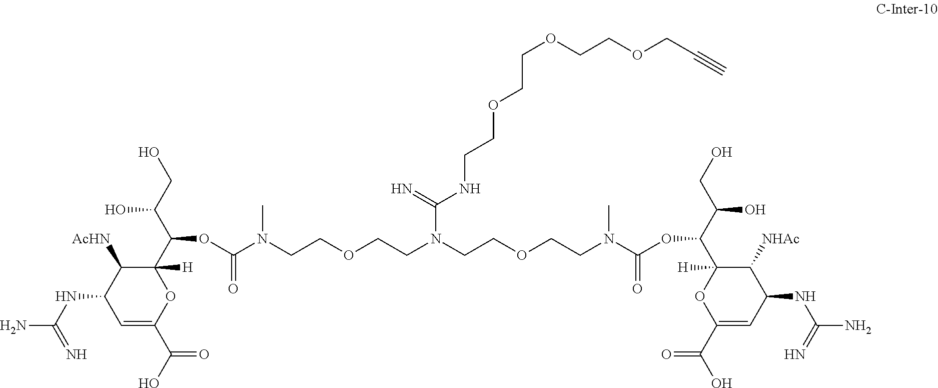 [SMILES: C#CCOCCOCCOCCNC(=N)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO]

### Step One: Synthesis of C-Inter-10-1

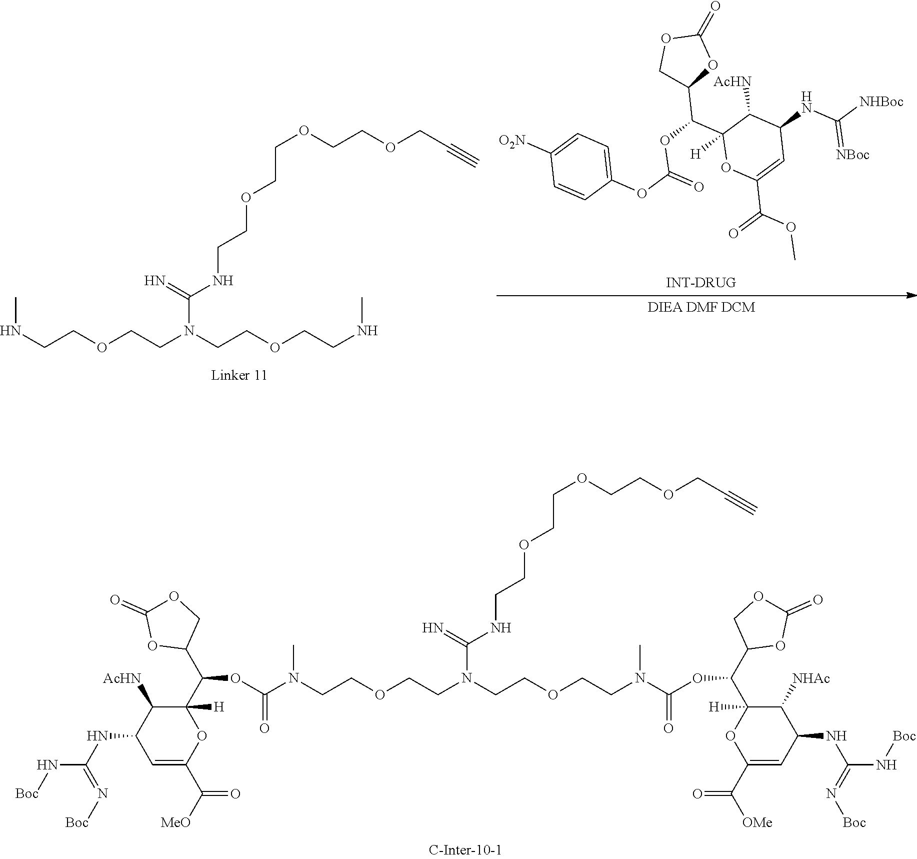

Linker 11 (calculated as 0.1 mmol) dissolved in DMF (2 ml) was added with DIEA (0.4 g, 3 mmol), to which INT-DRUG (147 mg, 0.2 mmol) dissolved in DCM (2 ml) was added dropwise. The mixture was stirred for 16 h at room temperature, and then concentrated under reduced pressure to obtain the crude product. The crude product was purified by reversed-phase column chromatography (acetonitrile/water), and freeze dried to obtain the white solid C-Inter-10-1 (70 mg). LCMS[M/2+1]+: 865.4.

### Step Five: Synthesis of C-Inter-10-2

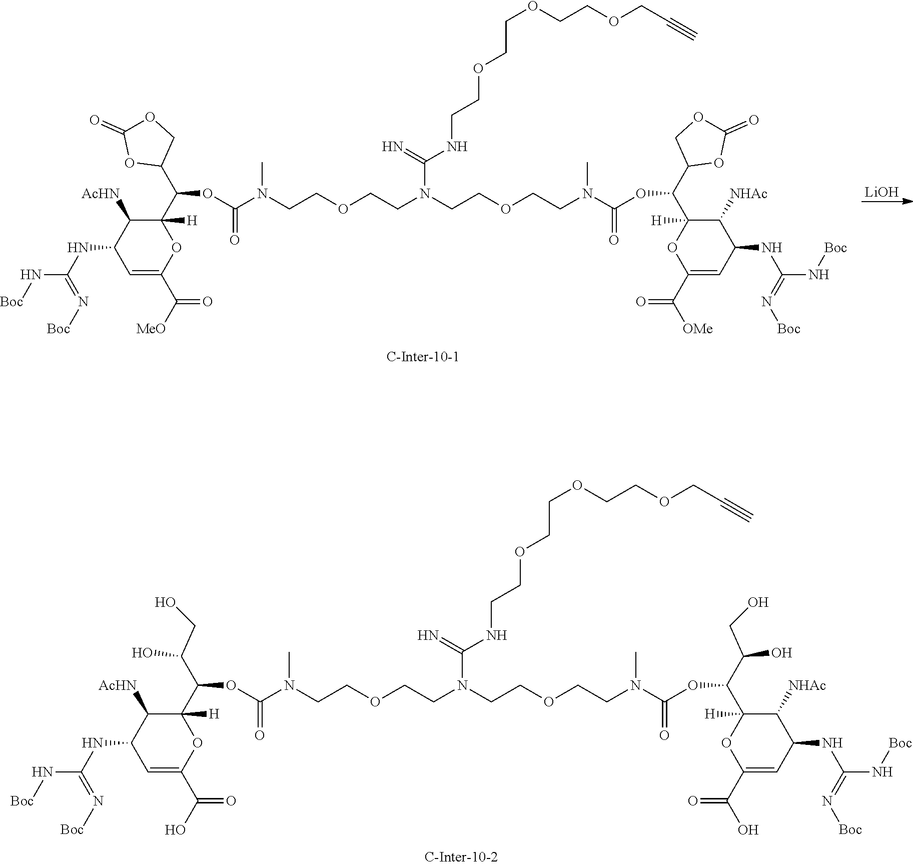 [SMILES: C#CCOCCOCCOCCNC(=N)N(CCOCCN(C)C(=O)O[C@H](C1COC(=O)O1)[C@@H]1OC(C(=O)OC)=C[C@H](N/C(=N/C(=O)OC(C)(C)C)NC(=O)OC(C)(C)C)[C@H]1NC(C)=O)CCOCCN(C)C(=O)O[C@H](C1COC(=O)O1)[C@@H]1OC(C(=O)OC)=C[C@H](N/C(=N/C(=O)OC(C)(C)C)NC(=O)OC(C)(C)C)[C@H]1NC(C)=O ; "LiOH" ; C#CCOCCOCCOCCNC(=N)N(CCOCCN(C)C(=O)O[C@@H]([C@@H]1OC(C(=O)O)=C[C@H](N/C(=N/C(=O)OC(C)(C)C)NC(=O)OC(C)(C)C)[C@H]1NC(C)=O)[C@H](O)CO)CCOCCN(C)C(=O)O[C@@H]([C@@H]1OC(C(=O)O)=C[C@H](N/C(=N/C(=O)OC(C)(C)C)NC(=O)OC(C)(C)C)[C@H]1NC(C)=O)[C@H](O)CO]

C-Inter-10-1 (70 mg, 0.043 mmol) dissolved in methanol (2 ml) was added with aqueous lithium hydroxide (0.22 mmol, 1 ml), which was stirred at room temperature for 1 h. After addition of acetic acid (0.3 mL), rotary evaporation was performed to give the crude product C-Inter-10-2 that was used directly in the next step.

### Step Six: Synthesis of C-Inter-10

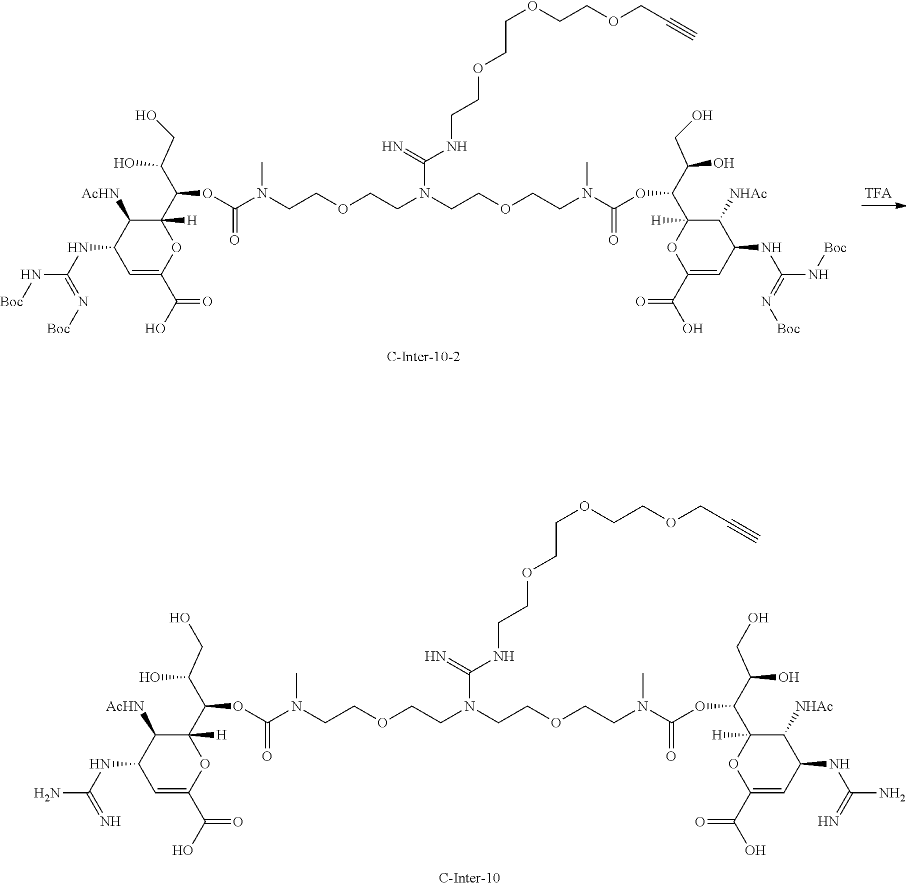 [SMILES: C#CCOCCOCCOCCNC(=N)N(CCOCCN(C)C(=O)O[C@@H]([C@@H]1OC(C(=O)O)=C[C@H](N/C(=N/C(=O)OC(C)(C)C)NC(=O)OC(C)(C)C)[C@H]1NC(C)=O)[C@H](O)CO)CCOCCN(C)C(=O)O[C@@H]([C@@H]1OC(C(=O)O)=C[C@H](N/C(=N/C(=O)OC(C)(C)C)NC(=O)OC(C)(C)C)[C@H]1NC(C)=O)[C@H](O)CO ; C#CCOCCOCCOCCNC(=N)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO]

The crude C-Inter-10-2 was placed in trifluoroacetic acid (1 ml) and stirred at room temperature for 30 min. After the reaction completed, rotary evaporation was performed to remove the solvent and give the crude product. The crude product was purified by reversed-column purification (acetonitrile/0.1% TFA aqueous solution) and freeze dried to give the C-Inter-10. LCMS[M+1]+:1148.5.

### Example 23: In Vitro Anti-Influenza Virus (Neuraminidase Inhibition Assay) Activity

### 1. Experimental Purpose:

To verify the effect of different neuraminidase inhibitors on the activity of different neuraminidases

### 2. Experimental Reagents and Consumables:

TABLE 1
Experimental reagents and consumables

| Product | Company | Cat.No. |
|---|---|---|
| 96-well microtiter plate | Greiner | 655077 |
| HTS black | | |
| 4-Methylumbelliferyl- | Sigma-Aldrich | M8639-25MG |
| N-acetyl-α-D-neuraminic | | |
| acid sodium salt hydrate | | |
| Tris | Sigma | 93362-500G |
| CaCl₂ | Greagent | 10043-52-4 |
| NaCl | Greagent | 7647-14-5 |

TABLE 2
Instruments

| Product | Company | Type |
|---|---|---|
| Microplate Reader | Tecan | infinite 200Pro |
| pH Meter | METTLER | Seven Compact ™ |
| | TOLEDO | |

TABLE 3
Neuraminidase

| Product | Company | Cat.No. | Batch No. |
|---|---|---|---|
| Influenza A H1N1 | Sino | 11058- | LC14OC2611 |
| (A/California/04/2009) | Biological | VNAHC | |
| Neuraminidase/NA (Active) | | | |
| Influenza A H3N2 | Sino | 40017- | LC05AU2801 |
| Neuraminidase/NA (Active) | Biological | VNAHC | |

TABLE 4
Positive control compounds (neuraminidase inhibitors)

| | | | | Molecular |
|---|---|---|---|---|
| Product | Company | Cat.No. | Batch No. | weight |
| Zanamivir | Adamas | 139110-80-8 | P1695633 | 332.31 |

### 3. Experimental Preparation

1) Assay buffer configuration: 50 mM Tris, 5 mM CaCl₂), 200 mM NaCl, pH 7.5.
   6.05 g of Tris, 0.56 g of CaCl₂ and 11.69 g of NaCl were weighed and dissolved in 800 ml of ultrapure water. The pH was adjusted to 7.5, and 1 L of water was added followed by filtration through a 0.22 micron membrane. The solution was packaged at 50 ml/tube and stored at 4° C.
2) Neuraminidase substrate storage solution: Neuraminidase substrate 4-Methylumbelliferyl-N-acetyl-α-D-neuraminic acid sodium salt hydrate was dissolved in water at the concentration of 20 mM, which was packaged at 100 ml/tube and stored at −20° C. in shade.
3) Different neuraminidases were dissolved according to the reagent instructions, which were packaged at 10 U/tube and stored at −80° C.
4) The neuraminidase inhibitor to be tested was dissolved in sterile water at the concentration of 5 mM, which was packaged at 50 l/tube and stored at −80° C.

### 4. Experimental Procedure

1) The different neuraminidases were diluted to 4 U/ml (4×) with Assay buffer, with a final concentration of 1 U/ml.
2) 25 μL of diluted neuraminidase solution was added to columns 2 to 12 of the 96-well plate, and 25 μL of Assay buffer was added to column 1.
3) The different neuraminidase inhibitors were diluted to 4 μM, with a final concentration of 1 μM. Then gradient dilutions for the diluted solution of each inhibitor were performed according to the following table.

TABLE 5
Gradient dilution for each neuraminidase inhibitor

| | | 4× | 1× |
|---|---|---|---|
| | | use CON | final CON |
| CON | Configuration Method | (nM) | (nM) |
| 1 | 600 μL 40000 nM zanamivir | 4000.00 | 1000.00 |
| 2 | 300 μL CON-1 + | 1333.33 | 333.33 |
| | 600 μL assay buffer | | |
| 3 | 300 μL CON-2 + | 444.44 | 111.11 |
| | 600 μL assay buffer | | |
| 4 | 300 μL CON-3 + | 148.15 | 37.04 |
| | 600 μL assay buffer | | |
| 5 | 300 μL CON-4 + | 49.38 | 12.35 |
| | 600 μL assay buffer | | |
| 6 | 300 μL CON-5 + | 16.46 | 4.12 |
| | 600 μL assay buffer | | |
| 7 | 300 μL CON-6 + | 5.49 | 1.37 |
| | 600 μL assay buffer | | |
| 8 | 300 μL CON-7 + | 1.83 | 0.46 |
| | 600 μL assay buffer | | |
| 9 | 300 μL CON-8 + | 0.61 | 0.15 |
| | 600 μL assay buffer | | |
| 10 | 300 μL CON-9 + | 0.20 | 0.05 |
| | 600 μL assay buffer | | |

4) 25 μL of neuraminidase inhibitors at concentration (CON) 1-10 in Table 4 was added to columns 2˜11 respectively, and 25 μl Assay buffer was added to columns 1 and 12.
5) The 96-well plate was covered with a sealing touch and incubated for 20 min at room temperature.
6) The Microplate Reader was turned on and warmed up to 37° C. Relevant parameters are set: excitation wavelength at 365 nm with a wave width of 9 nm; the emission wavelength of 450 nm with a wave width of 20 nm; Gain at 60.
7) Neuraminidase substrate diluted to 400 M was added to 96-well plate at 50 l/well, incubated at 37° C. for 20 min and then put into the Microplate Reader for reading. 8) Data analysis was performed using GraphPad Prism 8

### 5. Data Analysis:

1) Calculation of background value: average fluorescence value of BACKGROUND in column 1.
2) Calculation of neuraminidase enzyme activity: subtraction of the background value from the average fluorescence value of different neuraminidases at 1 U/ml in Column 12 subtracts the background value.
3) Calculation of enzyme activity for neuraminidase inhibitors at different concentrations: subtraction of the background value from the fluorescence value of each well in columns 2 to 11.
4) Effect of different concentrations of neuraminidase inhibitors on enzyme activity: enzyme activity of different concentrations of neuraminidase inhibitors/neuraminidase enzyme activity*100%
5) Calculation of IC50 using non-linear curve fitting, wherein the horizontal coordinate is the inhibitor concentration, and the vertical coordinate is the effect of using different concentrations of neuraminidase inhibitor on enzyme activity, in GraphPad Prism 8 software.

### 6. Summary of Data:

| | H1N1 | H3N2 |
|---|---|---|
| | (1 U/ml) | (1 U/ml) |
| Compound | IC₅₀(nM) | IC₅₀(nM) |
| Zanamivir | 1.00 | 4.06 |
| C-Inter-1 | 10.42 | 23.04 |
| C-Inter-2 | 14.18 | 44.33 |
| C-Inter-3 | 21.30 | 36.71 |
| C-Inter-4 | 48.67 | 158.60 |
| C-Inter-5 | 36.99 | 115.10 |
| C-Inter-6 | 11.95 | 24.94 |
| C-Inter-7 | 7.64 | 20.01 |
| C-Inter-10 | 10.76 | 19.15 |

The tested compounds all demonstrated high neuraminidase inhibitory activity without much activity loss due to the inclusion of the linker moiety. The inhibitory activities against H1N1 and H3N2 strains also reached the nM level, showing the worth of further development.

### Example 24: Influenza Virus-Mediated Cytopathic Inhibitory Activity

### 1. Experimental Method

In vitro cytopathic-based anti-IAV/IBV activity assay refers to quantify the effect of the tested compounds on IAV/IBV-induced cytopathic using MDCK cell lines.

### 2. Experimental Material

| Material | Origin | Batch No. |
|---|---|---|
| H1N1(A/PR/8/34) virus | ATCC | VR1469 |
| MDCK cell | ATCC | CCL-34 |
| 96-well plate | Corning | 3917 |
| Fetal Bovine Serum (FBS) | Hyclone | SH30406.05 |
| 100× Pen-Strep solution (P/S) | Hyclone | SV30010 |
| DMEM | Hyclone | SH30243.01 |
| TPCK treated trypsin | Sigma | T1426 |
| Cell-titer Glo | Promega | G7571 |

### 3. Experimental Procedure

1) MDCK cells were digested and diluted to 2×10⁵/mL with DME medium containing 2% FBS and 100 P/S, which was inoculated into 96-well plate at 50 μL per well and placed in an incubator for overnight culture.
2) The tested compounds were diluted to 2 μM that was then triple diluted with a total of 8 dilution ratios. 5 μL of diluted compound was added to each well and placed in the incubator for 1 h.
3) Influenza virus was diluted to 2,222 pfu/mL with DMEM medium containing 2% FBS, 1% P/S and 4 g/mL TPCK treated trypsin. 45 μL of virus was added to each well. A virus control group without compounds but with viruses and a cell control group without compounds and viruses were also configured. The cells were incubated in an incubator for 4 days.
4) 50 μL of Cell-titer Glo was added to each well, and the chemiluminescence was detected by a Microplate Reader.

### 4. Data Analysis: Calculate the Viral Inhibition Rate of the Compounds.

Inhibition rate=(value of tested compounds−average value of virus control)/(average value of cell control−average value of virus control)
The calculated inhibition rate was used to calculate the EC50 value by fitting the inhibition curve using the log(inhibitor) vs. response—Variable slope (four parameters) in the Nonlinear regression (curve fit) function of GraphPad Prism 8 software.

### 5. Summary of Results

| Compound | EC50 (nM) |
|---|---|
| Zanamivir | 39.69 |
| C-Inter-1 | 0.28 |
| C-Inter-2 | 0.42 |
| C-Inter-3 | 0.39 |
| C-Inter-5 | 0.45 |
| C-Inter-6 | 0.25 |
| C-Inter-7 | 0.57 |
| C-Inter-10 | 0.39 |

The test compounds demonstrated excellent in vitro cellular anti-influenza activity, all of which showed nearly 100-fold or more than 100-fold improvement in activity compared to Zanamivir positive molecule. The in vitro cellular anti-influenza activity demonstrates the strong anti-influenza activity of the listed molecules, showing the huge worth of further development.

### Example 25: Cytotoxicity Assay of Compounds

### 1. Experimental Method

Toxicity of the tested compounds to MDCK cells was quantified by MDCK cell lines.

### 2. Experimental Material

| Material | Origin | Batch No. |
|---|---|---|
| MDCK Cell | ATCC | CCL-34 |
| 96-well plate | Corning | 3917 |
| Fetal Bovine Serum (FBS) | Hyclone | SH30406.05 |
| 100× Pen-Strep solution | Hyclone | SV30010 |
| (P/S) | | |
| DMEM | Hyclone | SH30243.01 |
| TPCK treated trypsin | Sigma | T1426 |
| Cell-titer Glo | Promega | G7571 |

### 3. Experimental Procedure

1) MDCK cells were digested and diluted to 2×10⁵/mL with DME medium containing 2% FBS and 1% P/S, which was inoculated into 96-well plate at 50 μL per well and placed in an incubator for overnight culture.
2) The tested compounds were diluted to 2 mM that was then triple diluted with a total of 8 dilution ratios. 5 μL of diluted compound was added to each well and placed in the incubator for 1 h.
3) Each well was supplemented with 45 μL of DMEM medium containing 2% FBS and 1% P/S, and 4 μg/mL TPCK treated trypsin. The cell control without compound was also configured. The cells were incubated in an incubator for 4 days.
4) 50 μL of Cell-titer Glo was added to each well, and the chemiluminescence was detected by a Microplate Reader.

### 4. Data Analysis: Inhibition Rate of Compounds on Cell Growth

Inhibition rate=(average value of cell control−value of tested compounds)/average value of cell control

The calculated inhibition rate was used to calculate the EC50 value by fitting the inhibition curve with the log(inhibitor) vs. response—Variable slope (four parameters) in the Nonlinear regression (curve fit) function of GraphPad Prism 8 software.

### 5. Summary of Results

| Compound | CC50 (μM) |
|---|---|
| Zanamivir | >100 |
| C-Inter-1 | >100 |
| C-Inter-2 | >100 |
| C-Inter-3 | >100 |
| C-Inter-5 | >100 |
| C-Inter-6 | >100 |
| C-Inter-7 | >100 |
| C-Inter-10 | >100 |

The tested compounds demonstrated excellent in vitro cellular safety. The cytotoxicity of tested compounds was above 100 μM, which is similar to the that of Zanamivir positive molecule. The in vitro cytotoxicity assay demonstrated the excellent safety profile of the listed molecules, showing great value for further development.

### Example 26: Expression and Purification of hIgG1-Fc

### 1. Experimental Material

| Material | Producer | Batch No. |
|---|---|---|
| ExpiCHO-S cell | Thermo | A29127 |
| ExpiFectamine ™ | Thermo | A29130 |
| CHO Transfection kit | | |
| OptiPRO ™ SFM | Thermo | 12309019 |
| reduced-serum medium | | |

2. Expression of hIgG1-Fc
1) The day before transfection (day −1), the cells were diluted to a final density of 3-4×106 viable cells/mL and growed overnight.
2) On the day of transfection, the cell density should be 5-6×106 cells/mL, and cell viability must be >95%.
3) Transfect 0.8 μg of pcDNA3.1 plasmid expressing hIgG1-Fc per mL of cells, 0.8 μg plasmids were diluted in 40 μL of OptiPRO™ SFM Medium (4° C.) and mixed gently as 0.8 μg of pcDNA3.1 plasmid expressing hIgG1-Fc was transfected in each mL of cells
4) 3.2 μL of ExpiFectamine™ CHO was diluted with 37 μL of OptiPRO™ SFM medium (4° C.) and mixed gently.
5) The diluted ExpiFectamine™ CHO reagent was added to the diluted DNA, which was mixed gently and incubated for 1-5 min at room temperature.
6) The above complexes were added slowly to each shake flask.
7) The cells were placed in a shaker at 37° C. with 8% CO2 and the speed of 125 rpm.
8) After incubating the cells for 20 hours, for per mL of cells, 10 μL of ExpiCHO Transfection Enhancer and 400 μL of ExpiCHO feed were mixed at this ratio, which was then added to each culture flask.
9) The cell supernatant was harvested after eight days of incubation.
   3. Purification of hIgG1 Fc

### 1) Collection of Supernatant

The Fc protein fermentation broth that has been transfected to harvest conditions was transferred to the 50 ml centrifuge tube marked in order and name, and centrifuged at 10000 rpm/min for 5 min. The supernatant after centrifugation was poured into a new 50 ml centrifuge tube marked in name until the same Fc fermentation broth has been centrifuged and the supernatant has been collected.

### 2) Pretreatment

The gravity column was treated by immersion in 0.5 M NaOH solution. Pro A packing solution was added to the empty gravity column. When the solution in the gravity column was dripped completely, 0.1M NaOH solution was taken and added to the column. When the solution in the gravity column was dripped completely, pure water was taken and added to the column.

### 3) Equilibration Sampling

The column was equilibrated by adding 5-10 column volumes of Pro A Equilibration Buffer to the Pro A column. The supernatant to be purified from the centrifugation process was added to the Pro A column and the flow-through supernatant was collected into a clean 250 ml shake flask. After all the supernatant has passed through the column, 5-10 column volumes of Pro-A Equilibration Buffer was added until all the Equilibration Buffer has flowed through at the bottom.

### 4) Protein Elution

The washed column was sealed at the bottom with a clean plug, into which 2-3 column volumes of Pro-A/G elution buffer was added, and the packing material was suspended using a pipette and then kept still for 5 min. For preparation of elution, a new collection tube was prepared, to which a certain proportion of Tris neutralizer was added to prevent protein denaturation. The plug was removed to collect the liquid into the collection tube and the protein concentration was measured. The protein was stored in −80° C. refrigerator.

3. Characterization of hIgG1 Fc

According to the sequence information of the Fc described herein, the target Fc is obtained according to the expression purification steps of this embodiment.

| | | | | | | | Average | |
|---|---|---|---|---|---|---|---|---|
| SEQ | Expression | Final | Final | Final | | Purity | Molecular | |
| ID | Volume | Production | Concentration | Volume | Vehicle | (SEC_HPLC) | Weight | Isoelectric |
| NO | (L) | (mg) | (mg/L) | (mL) | (PBS) | (%) | (Da) | Point |
| 64 | 5 | 3919.5 | 26.13 | 150.0 | PH 7.4 | 99.66 | 55266.6 | 8.93 |
| 67 | 5 | 2810.8 | 23.62 | 119.0 | PH 7.4 | 99.68 | 55176.4 | 8.93 |

### Example 27: Synthesis and Characterization of Conjugate 1

### 1) Synthesis and Characterization of Conjugate 1

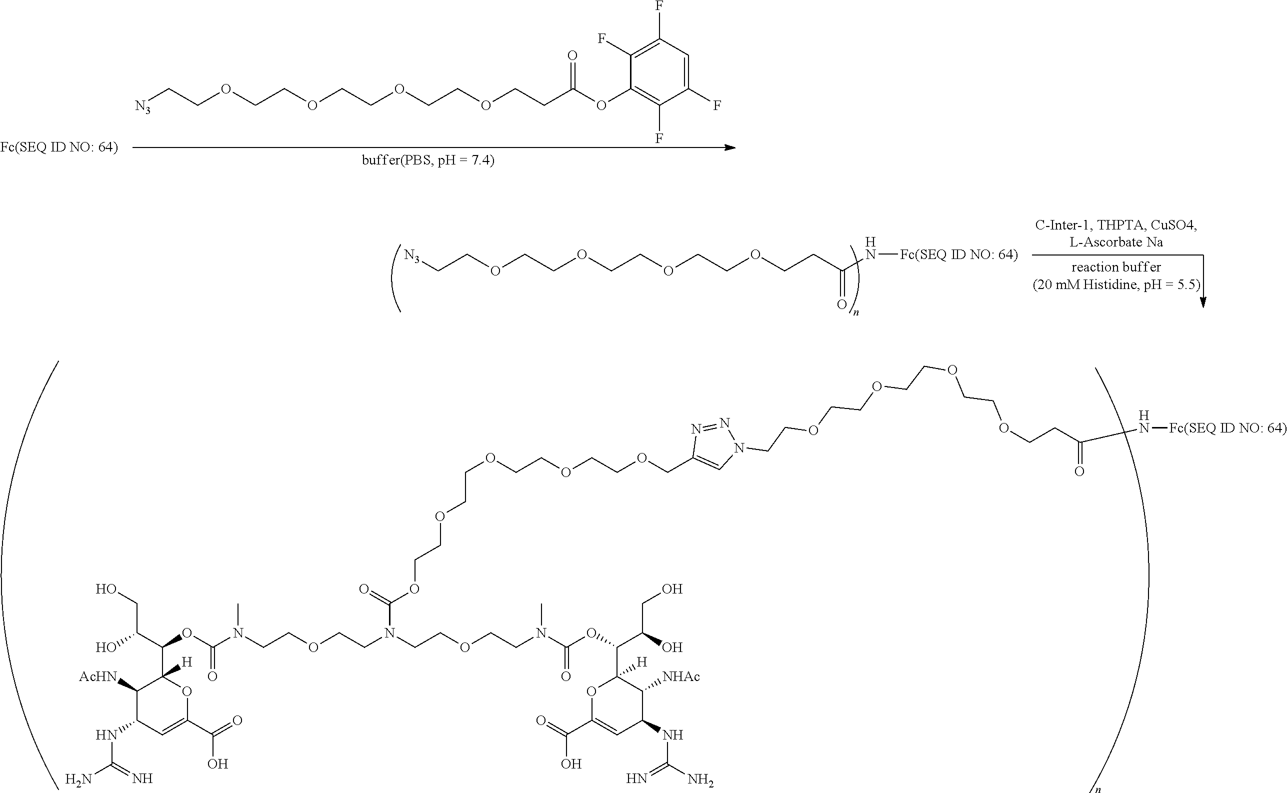

Step 1: The original buffer (PBS, pH 7.4) containing Fc (SEQ ID NO: 64) (45.4 mg) was pipetted into a 50 mL test tube. Reaction buffer (PBS, pH 7.4) was added to the test tube to give a final concentration of Fc of 14.97 mg/mL. Then, the active ester (azide-PEG4-C2-PFP ester) was added to give an active ester/Fc ratio of 9.95. The reaction vials were placed in a shaker incubator and reacted for 2 hours at 22° C. and 60 revolutions per minute. At the end of the reaction, the reaction solution was run through an Amicon ultra centrifugal filter (10K, 15 mL) to remove excess active ester, and the modified Fc (SEQ ID NO: 64)-Azide was displaced into buffer (PBS, pH 7.4)

Step 2: Original buffer (PBS, pH 7.4) containing Fc (SEQ ID NO: 64)-Azide was pipetted into a 50 mL tube. The reaction buffer (20 mM histidine, pH 5.5), 20 equivalents (equivalents refer to molar equivalents when not explicitly indicated herein and not inconsistent with the context) of THPTA (tris(3-hydroxypropyltriazolylmethyl)amine) (100 mM), 30 equivalents of sodium ascorbate (100 mM), 20 equivalents of copper sulfate (101 mM) and 12 equivalents of C-Inter-1 (20 mg/mL) were added to the test tube to give a final concentration of Fc (SEQ ID NO: 64)-Azide of 11.99 mg/mL. The reaction vials were placed in a shaker incubator and reacted for 30 minutes at 22° C. and 60 revolutions per minute. At the end of the reaction, the reaction solution was purified using an Amicon ultra centrifugal filter (10K, 15 mL) to obtain conjugate 1, with a concentration of 16.37 mg/mL as determined by UV, an actual weight of 24.5 mg, a DAR value of 4.34 as determined by LC-MS, a monomer content of 99.25% as determined by SEC-HPLC, a residual free drug content of less than 2%, and a endotoxin content of less than 0.098 EU/mg.

### 2) Characterization Method

### Amicon Ultra Centrifugal Filter Purification Method

Amicon Ultra centrifugal filter was used according to the following steps:
1) Wash the Amicon tube with sodium hydroxide solution (0.5 M). Allow the tubes to stand for 15 minutes.
2) Rinse the tubes three times with double-distilled water followed by addition of formulated buffer (20 mM histidine, pH 5.5).
3) Centrifuge and discard the filtered liquid.
4) Add the sample to the centrifuge tube, then centrifuge and discard the filtered liquid which were repeated 5 times.
5) Transfer the remaining solution in the Amicon tube to a new collection tube.

### UF/DF System Purification Method

UF/DF system purification method was performed according to the following steps:
1) Clean the system with sodium hydroxide solution (0.2 M). Allow the sodium hydroxide solution to flow for 5 minutes. Repeat this step once.
2) Rinse the collector twice with double-distilled water.
3) Rinse the collector twice with the prepared buffer until the pH of the filtrate is consistent with that of the buffer.
4) Add the sample to the collector and replace the buffer by at least 10 times the permeate volume.
5) Collect the solution in the collector.

### Concentration Determination of Conjugates

The concentration of the sample and final conjugate during the process were determined by the Nanodrop spectrophotometer in UV-Vis mode.
1) The baseline is configured at 750 nM.
2) Calculations based on Beer-Lambert law

$$\begin{matrix}A = E * c * 1 {A}_{280} = {E}_{280}^{Fc} * [ mAb ] * 1 + {E}_{280}^{LD} * [ LD ] * 1 \\ {A}_{252} = {E}_{252}^{Fc} * [ mAb ] * 1 + {E}_{252}^{LD} * [ LC ] * 1\end{matrix}$$

E: molar absorption coefficient;
c: molar concentration;
l: Light path (Nanodrop: 0.1 cm)

### Polymerization Determined by SEC-HPLC

Volume exclusion chromatography was performed at 25° C. using an Agilent 1260 series HPLC system and a TSK gel G3000SWXL volume exclusion column (7.8×300 mm, 5 m). The mobile phase is consisted of 78 mM KH2PO4, 122 mM K2HPO4, 250 mM KCl and 15% IPA, at a pH of 7.0±0.1. The flow rate was set at 0.75 mL/min. Each sample volume was 40-50 g. The samples were detected at 280 nm with a UV detector. The retention time of the aggregation peak was recorded based on its relative molecular weight. The aggregation level was determined by the relative area of the peaks.

### DAR Value Determined by LC-MS

LC-MS was run at 25° C. using an Agilent 6224 series HPLC system, TOF mass spectrometer and an Agilent PLRP-S 1000A column (8 μm, 50×2.1 mm). A double-distilled water solution of 0.05% trifluoroacetic acid was used as mobile phase A, and acetonitrile with 0.05% trifluoroacetic acid was used as mobile phase B. The flow rate was set at 0.5-0.4 mL/min. The sample volume was 2 μg. DAR values were calculated based on peak abundance of deconvolution quality.

### LC-MS Method for Determining DAR Value:

| Gas Temp. | 350° | | C. | |
|---|---|---|---|---|
| Drying Gas | 13 | | L/min | |
| Nebulizer | 45 | | psig | |
| VCap | 5000 | | V | |
| Fragmentor | 170 | | V | |
| Mass Range | 300-8000 | | m/z | |
| Acquisition Rate | 3 | | spectra/s | |
| Equipment | Agilent Technologies 6224 | | | |
| | TOF LC/MS | | | |
| Column | Agilent PLRP-S 1000A, | | | |
| | 8 μm, 50 × 2.1 mm | | | |
| Column Temp. | 80° | | C. | |
| Mobile phase | Phase A: 0.05% TFA in H₂O; | | | |
| | Phase B: 0.05% in Acetonitrile | | | |
| Flow rate | 0.4 | | mL/min | |
| Injection amount | 2 | | μg | |
| Detection wavelength | 280 nm, 214 nm, 650 nm | | | |
| | | | | Flow |
| Gradient | Time | A (%) | B (%) | (mL/min) |
| | 0.00 | 75 | 25 | 0.500 |
| | 0.70 | 66 | 34 | 0.400 |
| | 5.00 | 55 | 45 | 0.400 |
| | 6.00 | 10 | 90 | 0.400 |
| | 7.00 | 10 | 90 | 0.400 |
| | 7.10 | 75 | 25 | 0.400 |
| | 10.00 | 75 | 25 | 0.400 |

### Residual Free Drugs Determined by LC-MS

Residual free drug levels (mol/mol, free drug/bound drug) were determined by LC-MS. Two standards (2% & 50%) respectively containing free drug and Fc were prepared. The percentage of free drug was determined by comparing the EIC peak area of the residual free drug in the conjugate sample with that of standards.

Two standards were prepared according to the following steps:
C_{Fc} (1 mg/mL), DAR, MW_{Fc}, MW_{Drug} were known.

$$\begin{matrix}{M}_{Fc} = 1 / {MW}_{Fc} \\ {M}_{Total drug} \approx DAR / {MW}_{Fc} \\ a ) Moles of 2 \% standards \approx DAR * {M}_{Fc} * 2 \% \\ b ) Moles of 5 \% standards \approx DAR * {M}_{Fc} * 5 \%\end{matrix}$$

C=Concentration as determined by spectrophotometer (mg/mL)
DAR=Molar ratio of drug to antibody as determined by LC-MS
MW=molar weight

Taking 20% standard of the conjugate 5 as an example.

C_{Fc}: 1 mg/mL; DAR: 4.13; MW_{mAb}: 58160 g/mol; MW_{Drug}: 1150.2 g/mol

$$\begin{matrix}{M}_{Fc} = 1 / 58160 mol / L \\ {M}_{Total drug} \approx 1 / 58160 * DAR mol / L = 4.13 / 58160 mol / L \\ {M}_{2 \% standard drug} = 2 \% * 4.13 / 58160 mol / L = 1.42 u mol / L\end{matrix}$$

LP amount of 100 uL 2% standard: 1.42 umol/L*10⁻⁴ L*1150.2 g/mol=0.1633 ug C-Inter-7

The 200 standard was prepared by dissolving 0.1633 ug C-Inter-7 in 100 μL of 1 mg/mL Fc (SEQ ID NO: 64).

### LC-MS Method for Determination of Residual Free Drug:

| | | |
|---|---|---|
| Gas Temp. | 250° | C. |
| Drying Gas | 13 | L/min |
| Nebulizer | 45 | psig |
| VCap | 3500 | V |
| Fragmentor | 250 | V |
| Mass Range | 150-7000 | m/z |
| Acquisition Rate | 1 | spectra/s |

| Equipment | Agilent Technologies 6224 TOF LC/MS | |
|---|---|---|
| Column | Agilent PLRP-S 1000A, 8 μm, 50 × 2.1 mm | |
| Column Temp. | 80° | C. |

| Mobile phase | Phase A: 0.05% TFA in H₂O; | |
|---|---|---|
| | Phase B: 0.05% in Acetonitrile | |
| Flow rate | 0.4 | mL/min |
| Injection amount | 2 | μg |

| Detection | 280 nm, 214 nm, 650 nm | | | |
|---|---|---|---|---|
| wavelength | | | | |
| | | | | Flow |
| Gradient | Time | A (%) | B (%) | (mL/min) |
| | 0.00 | 95 | 25 | 0.400 |
| | 0.50 | 95 | 34 | 0.400 |
| | 2.00 | 70 | 45 | 0.400 |
| | 8.00 | 20 | 90 | 0.400 |
| | 8.50 | 10 | 90 | 0.400 |
| | 10.00 | 10 | 25 | 0.400 |
| | 10.01 | 95 | 25 | 0.400 |
| | 13.00 | 95 | 25 | 0.400 |

### Endotoxin Determination

The endotoxin levels of samples were determined by Endosafe®-PTS™ (Charles River, MCS150K) endotoxin detector. A 25 μL sample was pipetted into each of the four reservoirs of the PTS detector. In addition to the LAL reagent plus the positive reference control, the reader extracts the sample in the sample channel and mixes the sample with the LAL reagent. The samples combined with the colorimetric substrate and then were incubated. After mixing, the optical density of the wells is measured and analyzed based on an internally archived standard curve.

The endotoxin data for conjugates of embodiments in present application are shown in the following table.

| | Endo | UV Conc. | Endo |
|---|---|---|---|
| Sample | (EU/mL) | (mg/mL) | (EU/mg) |
| Conjugate 1 | <1.60 | 16.37 | <0.098 |
| Conjugate 2 | 6.18 | 18.6 | 0.332 |
| Conjugate 3 | <1.28 | 8.56 | <0.150 |
| Conjugate 4 | <1.5 | 21.69 | <0.069 |
| Conjugate 5 | 4.05 | 21.74 | 0.186 |
| Conjugate 6 | <1.8 | 19.67 | <0.092 |
| Conjugate 7 | <1.1 | 15.53 | <0.071 |
| Conjugate 8 | 1.2 | 13.59 | 0.088 |
| Conjugate 9 | <1.95 | 16.77 | <0.116 |

### Example 28: Synthesis and Characterization of Conjugate 2

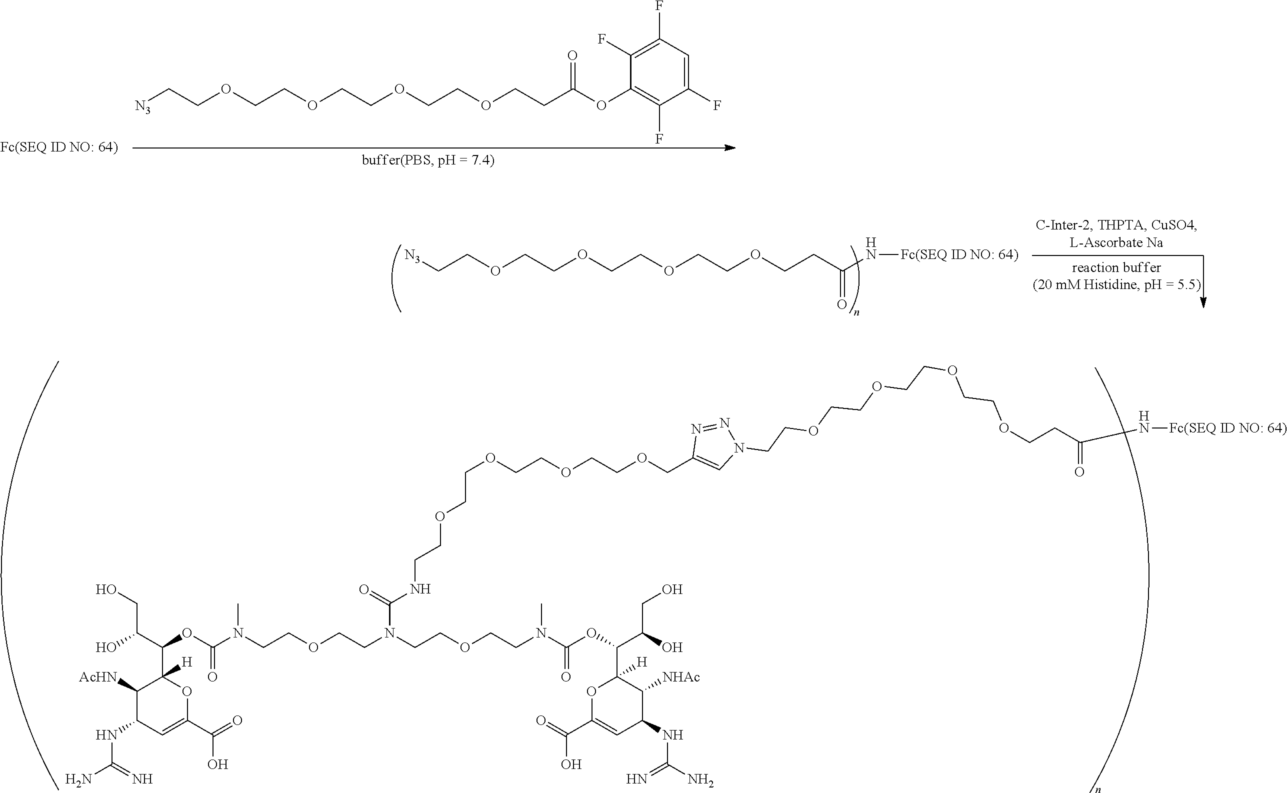

Step 1: The original buffer (PBS, pH 7.4) containing Fc (SEQ ID NO: 64) (140.0 mg) was pipetted into a 50 mL test tube. Reaction buffer (PBS, pH 7.4) was added to the test tube to give a final concentration of Fc of 14.97 mg/mL. Then, the active ester (azide-PEG4-C2-PFP ester) was added to give an active ester/Fc ratio of 9.4. The reaction vials were placed in a shaker incubator and reacted for 2 hours at 22° C. and 60 revolutions per minute. At the end of the reaction, the reaction solution was run through an Amicon ultra centrifugal filter (10K, 15 mL) to remove excess active ester, and the modified Fc (SEQ ID NO: 64)-Azide was displaced into buffer (PBS, pH 7.4)

Step 2: The original buffer (PBS, pH 7.4) containing Fc (SEQ ID NO: 64)-Azide was pipetted into a 50 mL tube. Reaction buffer (20 mM histidine, pH 5.5), 20 equivalents of THPTA (tris(3-hydroxypropyltriazolylmethyl)amine) (100 mM), 30 equivalents of sodium ascorbate (100 mM), 20 equivalents of copper sulfate (101 mM), and 12 equivalents of C-Inter-2 (20 mg/mL) were added to the test tube to make the Fc (SEQ ID NO: 64)-Azide to a final concentration of 11.99 mg/mL. The reaction vials were placed in a shaker incubator and reacted for 30 minutes at 22° C. and 60 revolutions per minute. At the end of the reaction, the reaction solution was purified by Amicon ultra centrifugal filter (10K, 15 mL) to obtain the conjugate 2.

Referring to the assay and analysis method of Example 27, the following data was measured for conjugate 2:
concentration of 18.6 mg/mL measured by UV, actual weight of 37.2 mg, DAR value of 4.04 determined by LC-MS, monomer content of 99.07% determined by SEC-HPLC, residual free drug content of less than 2%, and endotoxin content of less than 0.098 EU/mg.

### Example 29: Synthesis and Characterization of Conjugate 3

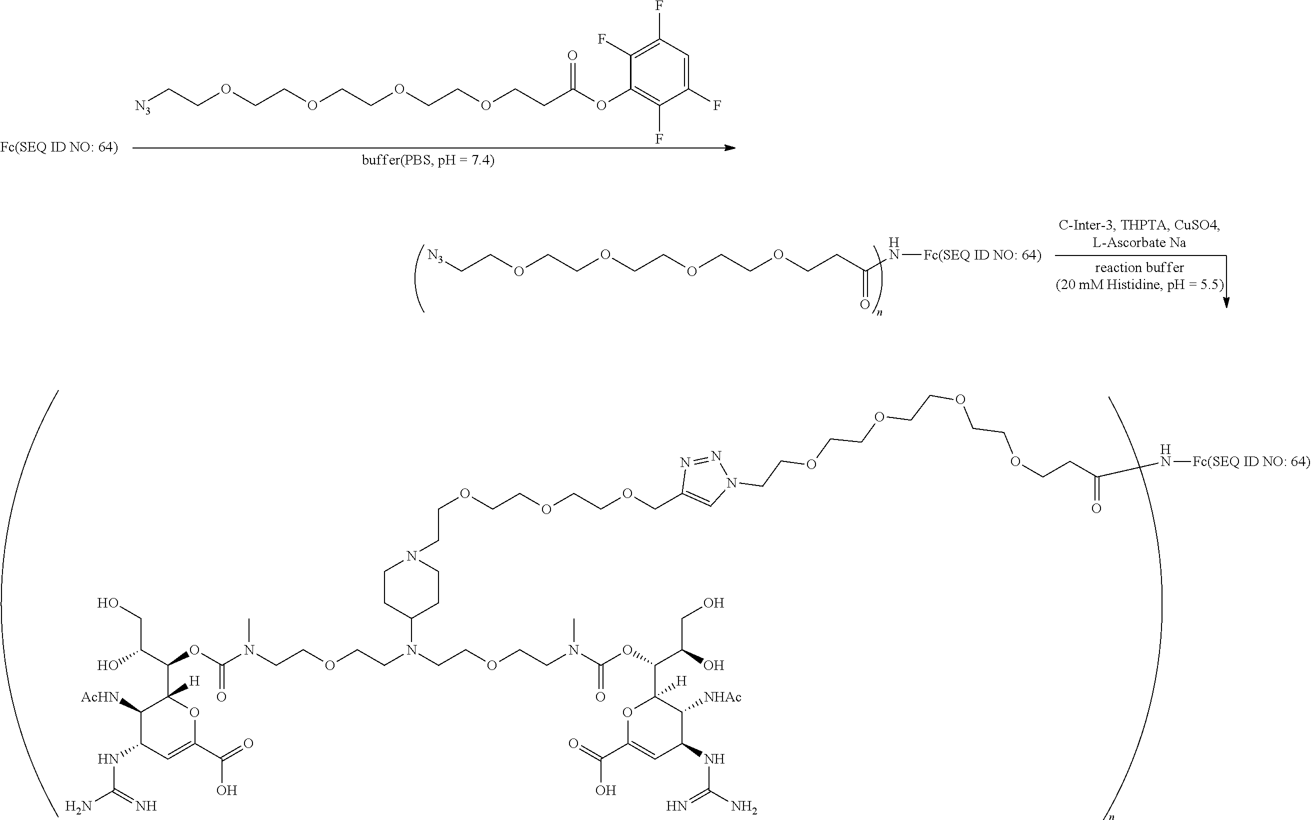

Step 1: The original buffer (PBS, pH 7.4) containing Fc (SEQ ID NO: 64) (46.5 mg) was pipetted into a 50 mL test tube. Reaction buffer (PBS, pH 7.4) was added to the test tube to give a final concentration of Fc of 14.97 mg/mL. Then the active ester (azide-PEG4-C2-PFP ester) was added to give an active ester/Fc ratio of 5.4/6.2. The reaction vials were placed in a shaking incubator and reacted for 2 hrs at 22° C. and 60 revolutions per minute. At the end of the reaction, the reaction solution was run through an Amicon ultra centrifugal filter (10K, 15 mL) to remove excess active ester, and the modified Fc (SEQ ID NO: 64)-Azide was displaced into buffer (PBS, pH 7.4).

Step 2: The original buffer (PBS, pH 7.4) containing Fc (SEQ ID NO: 64)-Azide was pipetted into a 50 mL tube. Reaction buffer (20 mM histidine, pH 5.5), 20 equivalents of THPTA (tris(3-hydroxypropyltriazolylmethyl)amine) (100 mM), 30 equivalents of sodium ascorbate (100 mM), 20 equivalents of copper sulfate (101 mM), and 12 equivalents of C-Inter-3 (20 mg/mL) were added to the test tube to make the Fc (SEQ ID NO: 64)-Azide to a final concentration of 11.99 mg/mL. the reaction vials were placed in a shaker incubator and reacted for 30 minutes at 22° C. and 60 revolutions per minute. At the end of the reaction, the reaction solution was purified using an Amicon ultra centrifugal filter (10K, 15 mL) to obtain the conjugate 3.

Referring to the assay and analysis method of Example 27, the following data was measured for conjugate 3:
concentration of 8.56 mg/mL measured by UV, actual weight of 30.7 mg, DAR value of 4.31 determined by LC-MS, monomer content of 99.10% determined by SEC-HPLC, residual free drug content of less than 5%, and endotoxin content of less than 0.150 EU/mg.

### Example 30: Synthesis and Characterization of Conjugate 4

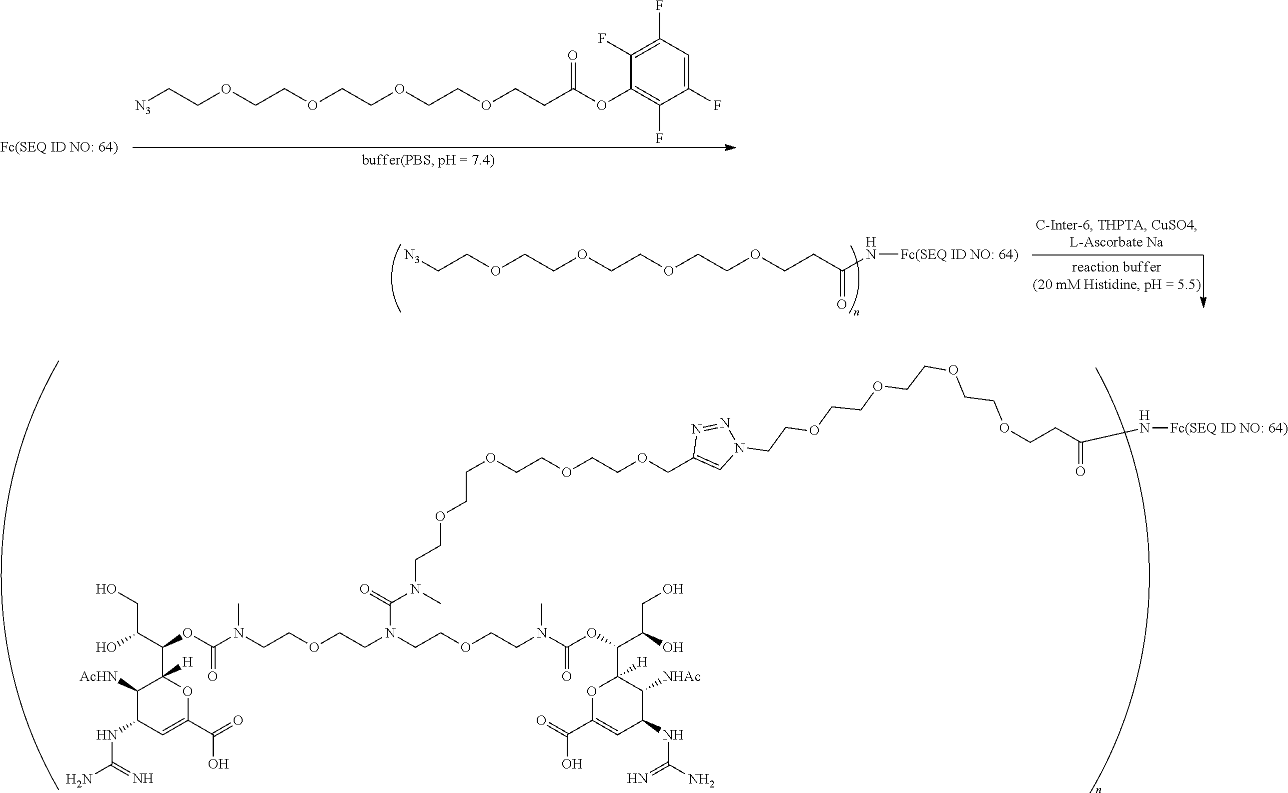

Step 1: The original buffer (PBS, pH 7.4) containing Fc (SEQ ID NO: 64) (234.2 mg) was pipetted into a 50 mL test tube. Reaction buffer (PBS, pH 7.4) was added to the test tube to give a final concentration of Fc of 14.97 mg/mL. Then, the active ester (azide-PEG4-C2-PFP ester) was added to give an active ester/Fc ratio of 5.7. The reaction vials were placed in a shaker incubator and reacted for 2 hrs at 22° C. and 60 revolutions per minute. At the end of the reaction, the reaction solution was run through an Amicon ultra centrifugal filter (10K, 15 mL) to remove excess active ester, and the modified Fc (SEQ ID NO: 64)-Azide was displaced into buffer (PBS, pH 7.4).

Step 2: The original buffer (PBS, pH 7.4) containing Fc (SEQ ID NO: 64)-Azide was pipetted into a 50 mL tube. Reaction buffer (20 mM histidine, pH 5.5), 20 equivalents of THPTA (tris(3-hydroxypropyltriazolylmethyl)amine) (100 mM), 30 equivalents of sodium ascorbate (100 mM), 20 equivalents of copper sulfate (101 mM), and 12 equivalents of C-Inter-6 (20 mg/mL) were added to the test tubes to make Fc (SEQ ID NO: 64)-Azide to a final concentration of 11.99 mg/mL. The reaction vials were placed in a shaker incubator and reacted for 30 minutes at 22° C. and 60 revolutions per minute. At the end of the reaction, the reaction solution was purified by Amicon ultra centrifugal filter (10K, 15 mL) to obtain conjugate 4.

Referring to the assay and analysis method of Example 27, the following data was measured for conjugate 4:
concentration of 21.69 mg/mL measured by UV, actual weight of 39.0 mg, DAR value of 4.24 determined by LC-MS, monomer content of 99.32% determined by SEC-HPLC, residual free drug content of less than 2%, and endotoxin content of less than 0.069 EU/mg.

### Example 31: Synthesis and Characterization of Conjugate 5

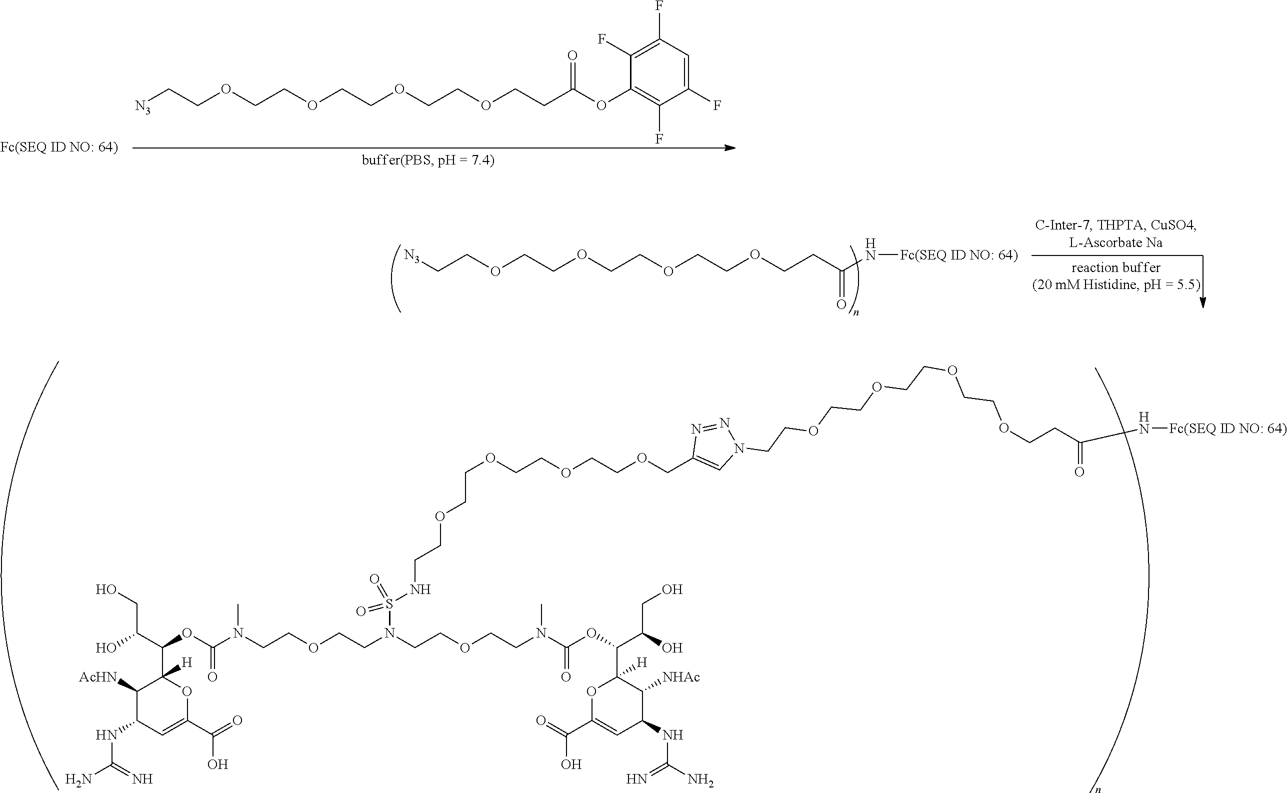

The original buffer of Fc (SEQ ID NO: 64)-Azide was obtained by referring to step 1 of conjugate synthesis in Example 27.

The original buffer (PBS, pH 7.4) containing Fc (SEQ ID NO: 64)-Azide was pipetted into a 50 mL test tube. Reaction buffer (20 mM histidine, pH 5.5), 20 equivalents of THPTA (tris(3-hydroxypropyltriazolylmethyl)amine) (100 mM), 30 equivalents of sodium ascorbate (100 mM), 20 equivalents of copper sulfate (101 mM), and 12 equivalents of C-Inter-7 (20 mg/mL) were added to the test tube to make the Fc (SEQ ID NO: 64)-Azide to a final concentration of 11.99 mg/mL. The reaction vials were placed in a shaker incubator and reacted for 30 minutes at 22° C. and 60 revolutions per minute. At the end of the reaction, the reaction solution was purified by Amicon ultra centrifugal filter (10K, 15 mL) to obtain conjugate 5.

Referring to the assay and analysis method of Example 27, the following data was measured for conjugate 5:
concentration of 21.74 mg/mL measured by UV, actual weight of 47.8 mg, DAR value of 4.16 determined by LC-MS, monomer content of 99.24% determined by SEC-HPLC, residual free drug content of less than 2%, and endotoxin content of 0.186 EU/mg EU/mg.

### Example 32: Synthesis and Characterization of Conjugate 6

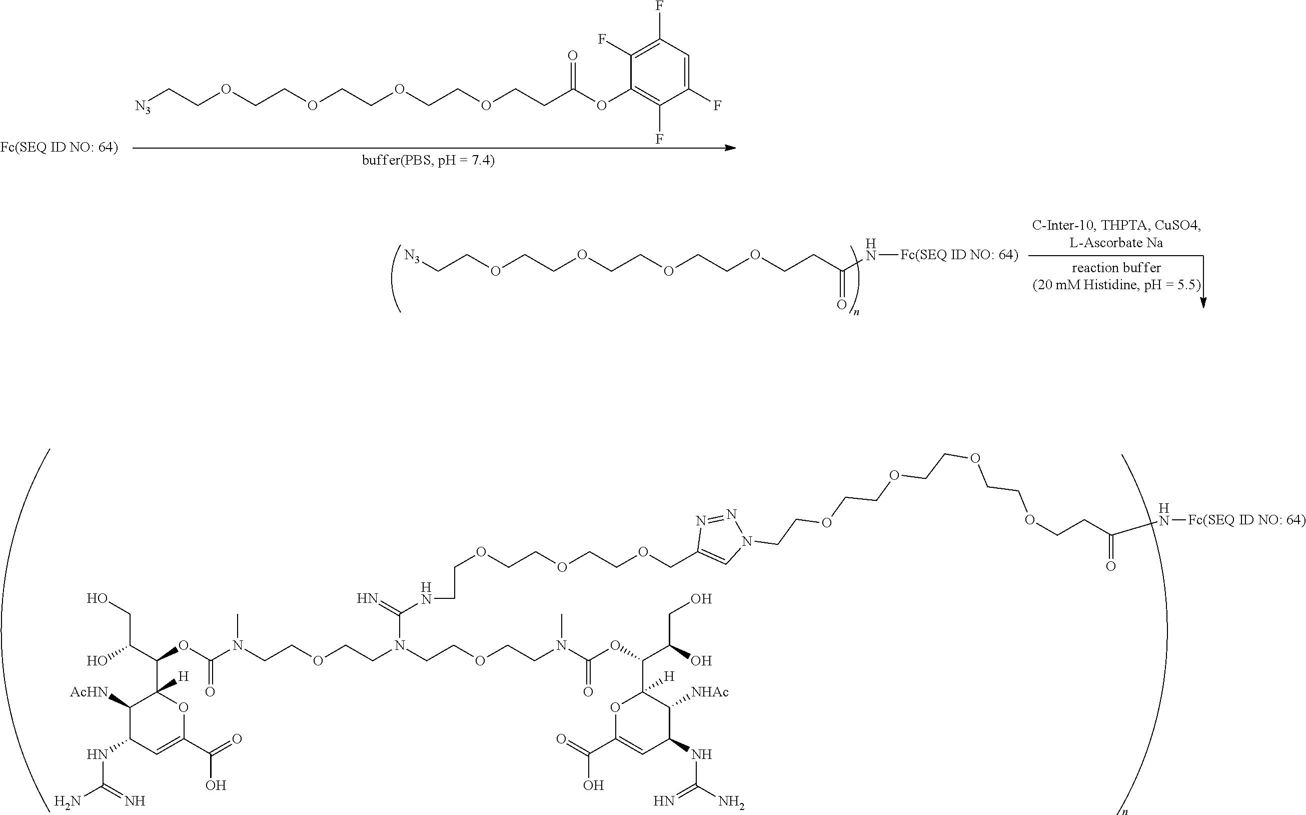

The original buffer of Fc (SEQ ID NO: 64)-Azide was obtained by referring to step 1 of conjugate synthesis in Example 27.

The original buffer (PBS, pH 7.4) containing Fc (SEQ ID NO: 64)-Azide was pipetted into a 50 mL tube. Reaction buffer (20 mM histidine, pH 5.5), 20 equivalents of THPTA (tris(3-hydroxypropyltriazolylmethyl)amine) (100 mM), 30 equivalents of sodium ascorbate (100 mM), 20 equivalents of copper sulfate (101 mM), and 12 equivalents of C-Inter-10 (20 mg/mL) were added to the test tubes to make Fc (SEQ ID NO: 64)-Azide to a final concentration of 11.99 mg/mL. The reaction vials were placed in a shaker incubator and reacted for 30 minutes at 22° C. and 60 revolutions per minute. At the end of the reaction, the reaction solution was purified using an Amicon ultra centrifugal filter (10K, 15 mL) to obtain the conjugate 6.

Referring to the assay and analysis method of Example 27, the following data was measured for conjugate 6:
concentration of 19.67 mg/mL measured by UV, actual weight of 39.3 mg, DAR value of 4.21 determined by LC-MS, monomer content of 99.31% determined by SEC-HPLC, residual free drug content of less than 2%, and endotoxin content of less than 0.092 EU/mg.

### Example 33: Synthesis and Characterization of Conjugate 7

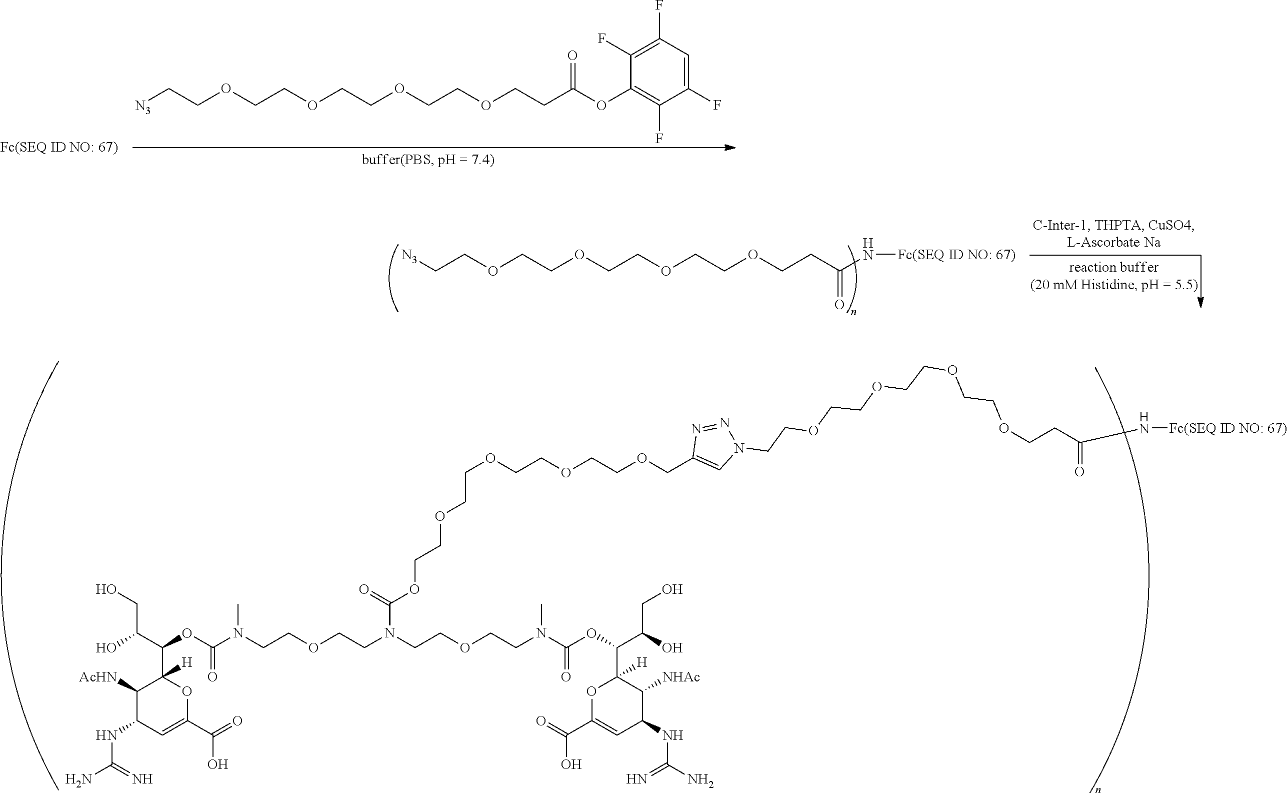

Step 1: The original buffer (PBS, pH 7.4) containing Fc (SEQ ID NO: 67) (378.9 mg) was pipetted into a 50 mL test tube. Reaction buffer (PBS, pH 7.4) was added to the test tube to make the final concentration of mAb 14.97 mg/mL. Then, the active ester (azide-PEG4-C2-PFP ester) was added to make the ratio of active ester/Fc 8.8. The reaction vials were placed in a shaker incubator and reacted for 2 hrs at 22° C. and 60 revolutions per minute. At the end of the reaction, the reaction solution was run through an Amicon ultra centrifugal filter (10K, 15 mL) to remove excess active ester, and the modified Fc (SEQ ID NO: 67)-Azide was displaced into buffer (PBS, pH 7.4).

Step 2: The original buffer (PBS, pH 7.4) containing Fc (SEQ ID NO: 67)-Azide was pipetted into a 50 mL tube. Reaction buffer (20 mM histidine, pH 5.5), 20 equivalents of THPTA (tris(3-hydroxypropyltriazolylmethyl)amine) (100 mM), 30 equivalents of sodium ascorbate (100 mM), 20 equivalents of copper sulfate (101 mM), and 18 equivalents of C-Inter-1 (20 mg/mL) were added to the test tubes to make Fc (SEQ ID NO: 67)-Azide to a final concentration of 11.99 mg/mL. The reaction vials were placed in a shaker incubator and reacted for 30 minutes at 22° C. and 60 revolutions per minute. At the end of the reaction, the reaction solution was purified using Amicon ultra centrifugal filter (10K, 15 mL) to obtain conjugate 7.

Referring to the assay and analysis method of Example 27, the following data was measured for conjugate 7:
concentration of 15.53 mg/mL measured by UV, actual weight of 74.0 mg, DAR value of 5.79 determined by LC-MS, monomer content of 98.77% determined by SEC-HPLC, residual free drug content of less than 2%, and endotoxin content of less than 0.071 EU/mg.

### Example 34: Synthesis and Characterization of Conjugate 8

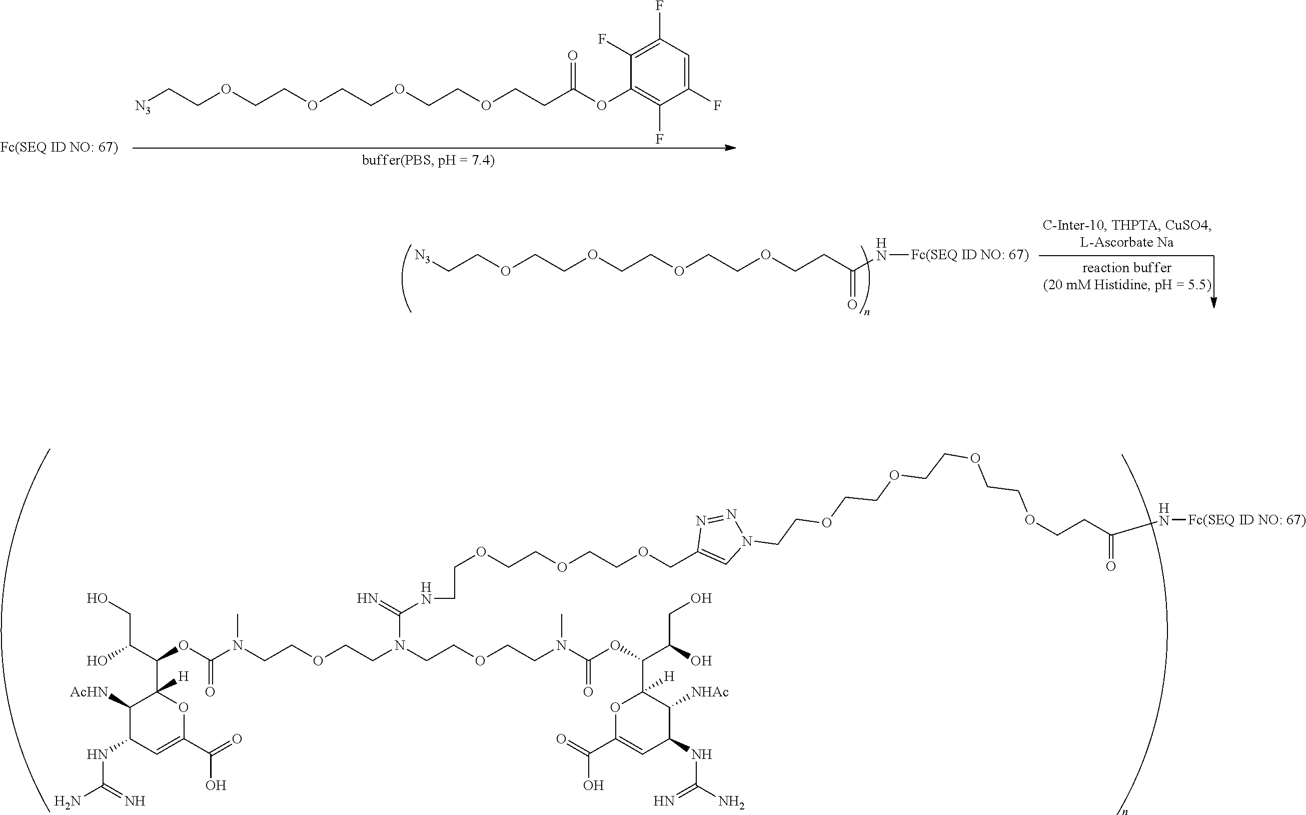

The original buffer of Fc (SEQ ID NO: 67)-Azide was obtained by referring to step 1 of conjugate synthesis in Example 33.

The original buffer (PBS, pH 7.4) containing Fc (SEQ ID NO: 67)-Azide was pipetted into a 50 mL tube. Reaction buffer (20 mM histidine, pH 5.5), 20 equivalents of THPTA (tris(3-hydroxypropyltriazolylmethyl)amine) (100 mM), 30 equivalents of sodium ascorbate (100 mM), 20 equivalents of copper sulfate (101 mM), and 18 equivalents of C-Inter-10 (20 mg/mL) were added to the test tubes to make Fc (SEQ ID NO: 67)-Azide to a final concentration of 11.99 mg/mL. The reaction vials were placed in a shaker incubator and reacted for 30 minutes at 22° C. and 60 revolutions per minute. At the end of the reaction, the reaction solution was purified using an Amicon ultra centrifugal filter (10K, 15 mL) to obtain the conjugate 8.

Referring to the assay and analysis method of Example 27, the following data was measured for conjugate 8:
concentration of 13.59 mg/mL measured by UV, actual weight of 54.0 mg, DAR value of 5.92 determined by LC-MS, monomer content of 98.88% determined by SEC-HPLC, residual free drug content of less than 2%, and endotoxin content of less than 0.088 EU/mg.

### Example 35: Synthesis and Characterization of Conjugate 9

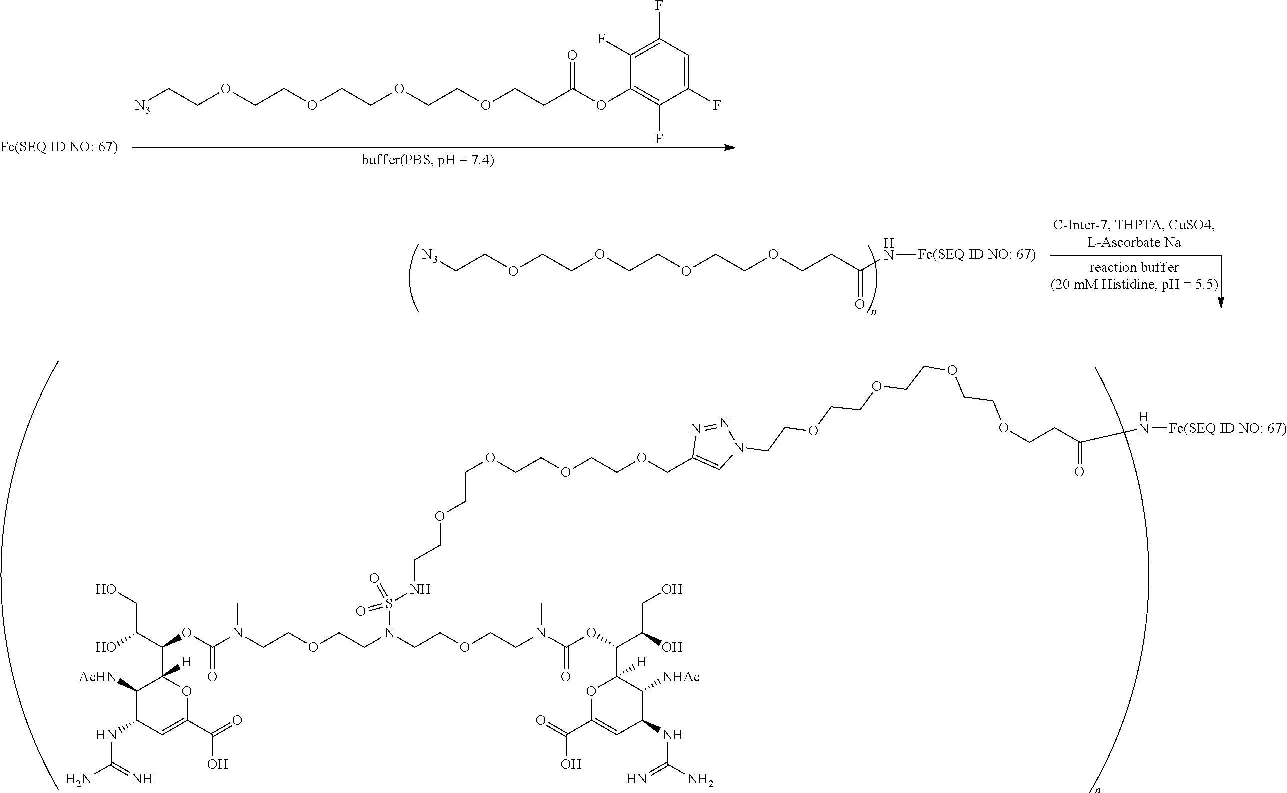

Step 1: The original buffer (PBS, pH 7.4) containing Fc (SEQ ID NO: 67) (3240 mg) was pipetted into a 150 mL reaction vial. Reaction buffer (PBS, pH 7.4) was added to the reaction vial to give a final concentration of Fc of 14.97 mg/mL. Then, the active ester (azide-PEG4-C2-PFP ester) was added to give an active ester/Fc ratio of 8.0/8.2. The reaction vial was placed in a shaker incubator and reacted for 2 hrs at 22° C. and 60 revolutions per minute. At the end of the reaction, the reaction solution was filtered through a UF/DF membrane filtration system to remove excess active ester, and the modified Fc (SEQ ID NO: 67)-Azide was displaced into buffer (PBS, pH 7.4).

Step 2: The original buffer (PBS, pH 7.4) containing Fc (SEQ ID NO: 67)-Azide was pipetted into a 150 mL tube. Reaction buffer (20 mM histidine, pH 5.5), 20 equivalents of THPTA (tris(3-hydroxypropyltriazolylmethyl)amine) (100 mM), 30 equivalents of sodium ascorbate (100 mM), 20 equivalents of copper sulfate (101 mM), and 18 equivalents of C-Inter-7 (20 mg/mL) were added to the test tubes to make Fc (SEQ ID NO: 67)-Azide to a final concentration of 11.99 mg/mL. The reaction vials were placed in a shaker incubator and reacted for 30 minutes at 22° C. and 60 revolutions per minute. At the end of the reaction, the reaction solution was purified by UF/DF membrane filtration system to obtain conjugate 9.

Referring to the assay and analysis method of Example 27, the following data was measured for conjugate 9:
concentration of 16.77 mg/mL measured by UV, actual weight of 3220 mg, DAR value of 5.86 determined by LC-MS, monomer content of 98.75% determined by SEC-HPLC, residual free drug content of less than 2%, and endotoxin content of less than 0.116 EU/mg.

### Example 36: In-Vitro Anti-Influenza Virus (Influenza Virus H1N1-Mediated Cytopathic Inhibition Assay) Activity of the Conjugate

### 1. Experimental Method

The in vitro cytopathic-based anti-IAV/IBV activity assay was used, which uses the MDCK cell line to quantify the effects of tested conjugates on IAV/IBV-induced cytopathies.

### 2. Experimental Material

| Material | Origin | Batch No. |
|---|---|---|
| H1N1(A/PR/8/34) | ATCC | VR1469 |
| MDCK cell | ATCC | CCL-34 |
| 96-well plate | Corning | 3917 |
| Fetal Bovine Serum (FBS) | Hyclone | SH30406.05 |
| 100× Pen-Strep solution (P/S) | Hyclone | SV30010 |
| DMEM | Hyclone | SH30243.01 |
| TPCK treated trypsin | Sigma | T1426 |
| Cell-titer Glo | Promega | G7571 |
| Zanamivir | Adamas | P1695633 |

### 3. Experimental Procedure

1) MDCK cells were digested and diluted to 2×10⁵/mL with DME medium containing 2% FBS and 1% P/S, which was inoculated into 96-well plate at 50 μL per well and placed in an incubator for overnight culture
2) The tested conjugates were diluted to 2 M that was then triple diluted with a total of 8 dilution ratios. 5 μL of diluted compound was added to each well and placed in the incubator for 1 h.
3) Influenza virus was diluted to 2,222 pfu/mL with DMEM medium containing 2% FBS, 1% P/S and 4 g/mL TPCK treated trypsin. 45 μL of virus was added to each well. A virus control group without compounds but with viruses and a cell control group without compounds and viruses were also configured. The cells were incubated in an incubator for 4 days.
4) 50 μL of Cell-titer Glo was added to each well, and the chemiluminescence was detected by a Microplate Reader.

### 4. Data Analysis: Calculate the Viral Inhibition Rate of the Conjugate.

$$Inhibition rate = ( value of tested conjugate - average value of virus control ) / ( average value of cell control - average value of virus control ) * 100 \%$$

The calculated inhibition rate was used to calculate the EC50 value by fitting the inhibition curve using the log(inhibitor) vs. response—Variable slope (four parameters) in the Nonlinear regression (curve fit) function of GraphPad Prism 8 software.

Reference molecule A and reference molecule B were respectively prepared according to the specific methods of Example 156 (Conjugate 45b) and Example 188 (Conjugate 46) in patent WO2021046549A1.

Reference molecule A has a purity (SEC-HPLC) of 99.2% and a DAR of 4.26.

Reference molecule B has a purity (SEC-HPLC) of 98.8% and a DAR of 5.87.

### 4. Summary of Results

| Compounds | DAR [symbol] | EC50 (nM) |
|---|---|---|
| Zanamivir | / | 45.7 |
| Reference molecule A | 4.26 | 0.6 |
| Conjugate 1 | 4.34 | 0.3 |
| Conjugate 2 | 4.04 | 0.2 |
| Conjugate 3 | 4.31 | 0.4 |
| Conjugate 4 | 4.24 | 0.2 |
| Conjugate 5 | 4.16 | 0.1 |
| Conjugate 6 | 4.21 | 0.2 |

Antiviral activity data for different Fc and higher
conjugating DAR values

| | | |
|---|---|---|
| Reference molecule B | 5.87 | 0.4 |
| Conjugate 7 | 5.79 | 0.07 |
| Conjugate 8 | 5.91 | 0.2 |
| Conjugate 9 | 5.86 | 0.07 |

As can be seen from the data, the tested conjugates showed a 76- to 652-fold improvement in antiviral activity compared to Zanamivir, demonstrating the superior in vitro anti-H1N1 viral activity.

At DAR values around 4.2, all the compounds in present application reflect better antiviral activity with lower EC50 values. Conjugate 5 showed a 6-fold improvement in antiviral activity as compared to reference molecule A.

At DAR values around 5.8, all the compounds in present application reflect better antiviral activity with lower EC50 values. Conjugates 7 and 9 showed nearly 6-fold improvement in antiviral activity as compared to reference molecule B.

The higher in vitro antiviral activity indicates better in vivo antiviral activity of conjugates in animals and in clinical humans.

### Example 37: In-Vitro Anti-Influenza Virus (H5N1, H7N9, Yamagata and Victoria Influenza Viruses) Activity of the Conjugate

### 1. Experimental Material

| Material | Origin | Batch No. |
|---|---|---|
| A/Chicken/Jiangsu/k0402/2010 | Yangzhou | / |
| (H5N1) | University | |
| A/Chicken/Eastern/JTC11/2013 | Yangzhou | / |
| (H7N9) | University | |
| B/Jiangsu/YZ08/2018 | Yangzhou | / |
| (Yamagata) | University | |
| B/YZ29/2019 (Victoria) | Yangzhou | / |
| | University | |
| MDCK cell | ATCC | Cat: CCL-34 |
| 96-well plate | Corning | Cat: 3917 |
| Fetal Bovine Serum (FBS) | Hyclone | Cat: SH30406.05 |
| 100× Pen-Strep solution (P/S) | Hyclone | Cat: SV30010 |
| DMEM | Hyclone | Cat: SH30243.01 |
| TPCK treated trypsin | Sigma | Cat: T1426 |
| Oseltamivir acid | MCE | HY-13318 |

### 2. Experimental Procedure

1) MDCK cells were digested and 2.5×10⁴ MDCK cells was inoculated into 96-well plate with DME medium containing 2% FBS and 1% P/S, which was cultured overnight.
2) The cell supernatant was discarded and the cells were infected with a dose of 0.0025 MOI of virus and placed in the incubator for 1 hour.
3) The virus not adsorbed to the cells was discarded, and each compound (the starting concentrations are shown in the table below) was triple diluted with eight gradients, and the diluted compound was added and incubated for 72 hours. The viral control group without compound was configured at the same time.

| Influenza strain | Conjugate 5 | Conjugate 9 |
|---|---|---|
| H5N1 | 30 nM | 30 nM |
| H7N9 | 30 nM | 30 nM |
| B/Yamagata | 10 μM | 10 μM |
| B/Victoria | 10 μM | 10 μM |

4) The effect of viral replication in each well was detected by hemagglutination assay and the viral titer was calculated according to the Reed-Muench method. The EC50 of each compound was calculated by the viral titer. The hemagglutination assay and the determination criteria were performed with reference to the WHO diagnostic criteria for influenza viruses.

### 3. Data Processing: Calculation of the Virus Inhibition Rate of Compounds

$$1 ) Inhibition rate = ( 1 - viral titer of the tested sample group / average value of the viral control group * 100 \% )$$

2) The calculated inhibition rate was used to calculate the EC50 value by fitting the inhibition curve with the log(inhibitor) vs. response—Variable slope (four parameters) in the Nonlinear regression (curve fit) function of GraphPad Prism 8 software.

### 4. Summary of Results

| | Oseltamivir | | Conjugate | Conjugate |
|---|---|---|---|---|
| Influenza strain | acid | Zanamivir | 5 | 9 |
| | EC₅₀ (nM) | | | |
| A/Chicken/Jiangsu/ | 210.0 | untested | 1.1 | 0.7 |
| k0402/2010 | | | | |
| (H5N1) | | | | |
| A/Chicken/Eastern/ | 540.0 | untested | 0.8 | 0.4 |
| JTC11/2013 | | | | |
| (H7N9) | | | | |
| B/Jiangsu/YZ08/2018 | 780.0 | 90.0 | 18.9 | 12.3 |
| (Yamagata) | | | | |
| B/YZ29/2019 | 710.0 | 59.0 | 58.9 | 10.7 |
| (Victoria) | | | | |

From the results, it can be seen that conjugate 5 and conjugate 9 each show a higher activity than the positive reference molecules in the tested influenza strains. It also demonstrated that the tested conjugates are broad-spectrum against influenza virus, which is of great clinical application value.

### Example 38: In-Vitro Anti-Influenza Virus (Clinically Resistant and Other Resistant Strains) Activity of the Conjugate

### 1. Experimental Material

| Material | Origin | Batch No. |
|---|---|---|
| A/California/2/2014 | ATCC | VR-1938 |
| (H3N2) | | |
| IFV B/Lee/40 | ATCC | VR-1535 |
| Oseltamivir-resistant | Constructed by | / |
| A/Weiss/43 (H1N1) | Wuxi New Drug | |
| | Development Co., | |
| | Ltd. Shanghai | |
| VX-787-resistant | Constructed by | / |
| A/PR/8/34 (H1N1) | Wuxi New Drug | |
| | Development Co., | |
| | Ltd. Shanghai | |
| Baloxavir-resistant | Constructed by | / |
| A/PR/8/34 (HIN1) | Wuxi New Drug | |
| | Development Co., | |
| | Ltd. Shanghai | |
| MDCK cell | ATCC | Cat: CCL-34 |
| 96-well plate | Corning | Cat: 3917 |
| Fetal Bovine Serum | Hyclone | Cat: SH30406.05 |
| (FBS) | | |
| 100× Pen-Strep solution | Hyclone | Cat: SV30010 |
| (P/S) | | |
| DMEM | Hyclone | Cat: SH30243.01 |
| TPCK treated trypsin | Sigma | Cat: T1426 |
| Cell-titer Glo | Promega | Cat: G7571 |

### 1. Experimental Procedure

MDCK cells were inoculated into 96-well cell culture plates at a density of 15,000 cells per well, a volume of 100 μL per well and incubated overnight in a 5% CO2, 37° C. incubator. The next day, 50 μL of gradient diluted compounds (3-fold serial dilution, 8 concentration points, double replicate wells) and 50 μL of virus were added to each well. For virus infection, trypsin at a final concentration of 2.5 μg/ml was added to the experimental culture solution. The total volume of culture medium was 200 μL per well. Cells were continued to be cultured at 5% CO2, 35° C. or 37° C. for 5 days until the obvious cellular pathology occurred in the virus-infected control wells without compounds. Then, cell viability was measured in each well using CellTiter-Ge, a cell viability assay reagent. If the cell viability of the compound-tested wells is higher than that of the virus-infected control wells, i.e., the CPE is attenuated, indicating the inhibitory effect of compound on the tested virus.

| | Starting concentration of test | | | | | |
|---|---|---|---|---|---|---|
| | | | | Oseltamivir | Conjugate | Conjugate |
| Tested virus strain | Zanamivir | Baloxavir | VX-787 | acid | 5 | 9 |
| IFV B/Lee/40 | 10 μM | 100 nM | untested | 100 μM | 100 nM | 100 nM |
| IFV A/California/2/ | 30 nM | 100 nM | untested | 100 μM | 30 nM | 30 nM |
| 2014 (H3N2) | | | | | | |
| Oseltamivir-resistant | 30 nM | 100 nM | untested | 100 μM | 30 nM | 30 nM |
| A/Weiss/43 (H1N1) | | | | | | |
| Baloxavir-resistant | 30 nM | 1000 nM | untested | 100 μM | 30 nM | 30 nM |
| A/PR/8/34 (H1N1) | | | | | | |
| VX-787-resistant | 30 nM | 100 nM | 10 μM | 100 μM | 30 nM | 30 nM |
| A/PR/8/34 (H1N1) | | | | | | |

### 3. Data Processing:

The viral inhibition rate of the compounds was first calculated.

$$Inhibition rate ( \% ) = ( reading of test well - average value of virus - infected control ) / ( average value of cell control - average value of virus - infected control ) \times 100$$

The calculated inhibition rate was used to calculate the EC50 value by fitting the inhibition curve using the log(inhibitor) vs. response—Variable slope (four parameters) in the Nonlinear regression (curve fit) function of GraphPad Prism 8 software.

### 4. Summary of Results

| Influenza strain | Oseltamivir acid | Zanamivir | Baloxavir | Conjugate 5 | Conjugate 9 |
|---|---|---|---|---|---|
| | EC₅₀ (nM) | | | | |
| IFV B/Lee/40 | 801.1 | 57.5 | 1.47 | 1.13 | 0.63 |
| IFV | >100,000 | >10,000 | 0.39 | 0.92 | 0.66 |
| A/California/2/2014 | | | | | |
| (H3N2) | | | | | |
| Oseltamivir-resistant | >100,000 | 24.4 | 0.69 | 8.61 | 8.51 |
| A/Weiss/43 (H1N1) | | | | | |
| Baloxavir-resistant | 2384.0 | 1391.0 | 95.0 | 4.17 | 2.57 |
| A/PR/8/34 (H1N1) | | | | | |
| VX-787-resistant | 1062.0 | 166.8 | 0.58 | 0.91 | 0.59 |
| A/PR/8/34 (H1N1) | | | | | |

From the results, it can be seen that conjugate 5 and conjugate 9 each exhibited a high antiviral activity in the tested influenza strains, with a EC50 value within 10 nM. Meanwhile, it also demonstrated that the tested conjugates are broad-spectrum against influenza virus, especially the maintained high antiviral activity for the clinically resistant strains, indicating the great clinical application value.

### Example 39: Pharmacokinetic Study of Conjugate 5 in Mice

### 1. Experimental Information

Pharmacokinetic studies were performed using male CD-1 (ICR) mice. The ELISA assay was performed for blood samples, and the specific experimental information and design are shown in the table below.

| Test | | | |
|---|---|---|---|
| compounds | Conjugate 5 | | |
| Administration | Intravenous | Subcutaneous | intramuscular |
| route | (IV) | injection (SC) | injection (IM) |
| Dosage | 5.0 | 5.0 | 5.0 |
| (mg/kg) | | | |
| Vehicle | 1 mg/mL | 1 mg/mL | 2 mg/mL |
| | dissolved in | dissolved in | dissolved in |
| | 1× PBS (pH 7.4) | 1× PBS (pH 7.4) | 1× PBS (pH 7.4) |
| Assay | ELISA | ELISA | ELISA |
| Blood | 0, 0.083, 1, 3, | 0, 0.25, 1, 3, | 0, 0.25, 1, 3, 5, |
| collection | 5, 24, 48, 72, | 5, 24, 48, 72, | 24, 48, 72, 96, |
| point design | 96, 168 h | 96, 168 h | 168 h |
| No. of animals | 6 | 6 | 6 |

### 2. Critical Experimental Material

| Classification | Material | Batch No. | Provider |
|---|---|---|---|
| Capture agent | Conjugate capture | 40017-VNAHC | Sino |
| | agent | | biological |
| HRP | anti-human-IgG- | 151886 | Jackson |
| | HRP | | |
| Wash buffer | 0.1% PBST | WB-20220314-GKS | Wuxi App |
| | | | Tec |
| Coating | CBS | CB-20220314-GKS | Wuxi App |
| Buffer | | | Tec |
| Blocking | 5% BSA in 0.1% | BB-20220316-GKS | Wuxi App |
| buffer (BSA) | PBST | | Tec |
| Assay Buffer | 1% BSA in 0.1% | AB-20220314-SWZ | Wuxi App |
| | PBST | | Tec |
| TMB | TMB Substrate Kit | 10537557 | SeraCare ™ |
| Termination | 2N H₂SO₄ | SS-20220219-YJC | Wuxi App |
| solution | | | Tec |

### 3. Detection Steps

Method description: The conjugate concentration in plasma of CD-1 mouse was determined by ELISA. The lower limit of quantification (LLOQ) of the conjugate was 50.0 ng/mL, while the upper limit of quantification (ULOQ) was 3000 ng/mL. The procedure is described below:
1) Blood was taken at the designed time points, and the corresponding test samples were prepared with EDTA-K2 as the anticoagulant.
2) 100 μL of coating solution was added to a 96-well microtiter plate that was sealed and incubated overnight at 2-8° C. before use.
3) The plate was washed 5 times with wash buffer. (300 μL/well).
4) The plate was blocked by adding 300 μL of blocking buffer to each well. then sealed and incubated at room temperature for 2 hours without shaking.
5) Step 2 was repeated.
6) 100 μL of standard solution and assay sample were added to each well. The plate was sealed and incubated at room temperature oscillation of 450 RPM for 2 hours±10 minutes.
7) Step 2 was repeated.
8) 100 μL of HRP solution was added to each well. The plate was sealed and incubated at room temperature oscillation of 450 RPM for 1 hour±10 minutes.
9) Step 2 was repeated.
10) 100 μL of TMB was added to each well. The plate was incubated at room temperature for 5-20 minutes.
11) 100 μL of termination solution was added to each well.
12) Plates were read within 30 minutes at 450 nm and 630 nm using a SpectraMax M5e/M5/Plus 384 microplate reader.
13) The reading of the standard solution was converted to the corresponding concentration of the conjugate.
14) Data processing is performed using Watson LIMS V7.6 or SoftMax Pro GxP 7.0.3 and Microsoft Excel 2007 or higher.

### 4. Summary of Results

PK Parameters of Conjugate 5

| | IV | SC | IM |
|---|---|---|---|
| PK Parameters | Mean plasma | Mean plasma | Mean plasma |
| C₀ (ng/ml) | 122965 | — | — |
| Cₘₐₓ (ng/ml) | — | 26633 | 32233 |
| Tₘₐₓ (h) | — | 48.0 | 24.0 |
| T_{1/2} (h) | 181 | 155 | 109 |
| Cl(mL/min/kg) | 0.00889 | — | — |
| Tₗₐₛₜ (h) | 169 | 168 | 168 |
| AUC₀₋ₗₐₛₜ (ng · h/mL) | 4572892 | 3381394 | 3836941 |
| AUC_{0-inf} (ng · h/mL) | 9378125 | 6629459 | 6044639 |
| MRT₀₋ₗₐₛₜ (h) | 68.4 | 82.9 | 75.3 |
| MRT_{0-inf} (h) | 254 | 234 | 167 |
| AUC_{Extra} (%) | 51.2 | 49.0 | 36.5 |
| AUMC_{Extra} (%) | 86.9 | 81.9 | 71.3 |
| Bioavailability (%) | — | 73.9 | 83.9 |

Pharmacokinetic data from mice showed that conjugate 5 has a long half-life and all three routes of administration reached the half-life of more than 100 hours. Meanwhile, all three routes of administration had excellent in vivo exposure. The above data suggests a long half-life and high exposure in the clinic for conjugate 5, enabling the excellent antiviral activity.

### Example 40: Pharmacokinetic Study of Conjugate 9 in Rat

Pharmacokinetic studies of the conjugate were performed using male SD rats. The ELISA assay was performed for blood samples, and the specific experimental information and design are shown in the table below.

| Test compounds | Conjugate 9 | |
|---|---|---|
| Administration route | Intravenous (IV) | |
| Dosage (mg/kg) | 15 | 50 |
| Vehicle | 3 mg/mL sample | 10 mg/mL sample |
| | dissolved in 1× volume | dissolved in 1× volume |
| | of PBS (pH 7.4) | of PBS (pH 7.4) |
| Assay | ELISA | ELISA |
| Blood collection | 0, 0.083, 0.25, 0.5, 1, 4, 8, 24, 72, 120, 168, 336 h_{∘} | |
| point design | | |
| No. of animals | 6 | 6 |

### 2. Critical Experimental Material

| Classification | Material | Batch No. | Provider |
|---|---|---|---|
| Capture agent | Conjugate capture | 40017-VNAHC | Sino |
| | agent | | biological |
| HRP | anti-human-IgG-HRP | 151886 | Jackson |
| Titration plate | 96-well micro titration | 468667 | Thermo |
| | plate | | |
| Buffer | Phosphate Buffer | PBS-0061 | MXB |
| | Powder | | Biotechnologies |
| Reagent | Bovine serum albumin | BSAS1.0 | BovoGen |
| | (BSA) | | |

Materials for which detailed information is not provided can be referred to Example 39.

### 3. Detection Steps

1) Blood was taken at the designed time points, and the corresponding test samples were prepared with EDTA-K2 as the anticoagulant.
2) 100 μL of coating solution was added to a 96-well microtiter plate that was sealed and incubated overnight at 2-8° C. before use.
3) The plate was washed 3 times with wash buffer. (300 μL/well)
4) The plate was blocked by adding 300 μL of blocking buffer to each well. then sealed and incubated at room temperature for 2 hours without shaking.
5) Step 2 was repeated.
6) 100 μL of standard solution/QC and assay sample were added to each well. The plate was sealed and incubated at room temperature oscillation of 450 RPM for 1 hour±10 minutes.
7) Step 2 was repeated.
8) 100 μL of HRP solution was added to each well. The plate was sealed and incubated at room temperature oscillation of 450 RPM for 1 hour±10 minutes.
9) Step 2 was repeated.
10) 100 μL of TMB was added to each well. The plate was incubated at room temperature for 5-20 minutes.
11) 100 μL of 2N H₂SO₄ was added to each well.
12) Plates were read within 30 minutes at 450 nm and 630 nm using a SpectraMax M5e/M5/Plus 384 microplate reader.
13) The reading of the standard solution was converted to the corresponding concentration of the conjugate
14) Data processing is performed using Watson LIMS V7.6 or SoftMax Pro GxP 7.0.3 and Microsoft Excel 2007 or higher.

### 4. Summary of Results

PK Parameters of Conjugate 9

| | IV (15 mg/kg) | IV (50 mg/kg) |
|---|---|---|
| PK Parameters | Mean plasma | Mean plasma |
| C₀ (ng/mL) | 277333 | 1295190 |
| T_{1/2} (h) | 177 | 183 |
| V_{dss} (L/kg) | 0.161 | 0.138 |
| Cl(mL/min/kg) | 0.0108 | 0.00930 |
| Tₗₐₛₜ (h) | 336 | 336 |
| AUC₀₋ₗₐₛₜ (ng · h/mL) | 16995540 | 65853090 |
| AUC_{0-inf} (ng · h/mL) | 23076300 | 89619410 |
| MRT₀₋ₗₐₛₜ (h) | 125 | 120 |
| MRT_{0-inf} (h) | 248 | 247 |
| AUC_{Extra} (%) | 26.4 | 26.5 |
| AUMC_{Extra} (%) | 63.0 | 64.4 |

Pharmacokinetic data from rat showed that conjugate 9 has a long half-life and the IV administration achieved the half-life of more than 150 hours under both dosages. Meanwhile, the exposure increased in a dose-dependent manner at the two dosages. The above data suggests a long half-life and high exposure in the clinic for conjugate 9, enabling the excellent antiviral activity

### Example 41. Serum Half-life in Mice (t_{1/2})

Pharmacokinetic (PK) studies were performed using CD-1 mice (Charles River Laboratories), weighing between 20-22 g. Each mouse was injected with 50 mg/kg of the compound to be tested (10 ml/kg dose volume) intravenously in the tail. All animals were under IACUC standard experimental conditions. At various time after administration, the mice were executed and blood was taken (EDTA anticoagulated tubes). Plasma was taken after centrifugation (2000×g, 10 min) of whole blood to analyze the concentration of the compound to be tested.

Plasma concentrations of the compound to be tested were measured by sandwich ELISA: ELISA plates were coated with viral surface proteins (influenza neuraminidase, respiratory syncytial virus F protein, and HIV surface glycoprotein gp120/gp41) targeted by the AVC compound, and were sealed in 2% bovine serum PBS for 1 hr at room temperature, and then added with the plasma samples diluted in serial. The horseradish peroxidase-labeled secondary antibody against human IgG-Fc is used for detection, and TMB substrate was added for color development for 20 min. Then, the same volume of reaction termination solution was added, and the absorbance value at OD450 nm was read. The concentration of the compound to be tested was calculated by fitting an S-curve drawn using a 4-parameter equation.

### Example 42: Mouse Prophylactic Model for Lethal Dose Influenza Virus

H1N1 Model: Female BALB/c mice aged 6-8 weeks were infected using a lethal dose of influenza virus strain H1N1 A/Texas/36/91. On day 0, inoculation with nasal drops was performed at a dose of 1×LD₉₀. Intravenous administration was performed 4 hours prior to inoculation. Oral Oseltamivir administration was used as a positive control for the experiment (Administered 8 hours after infection, 50 mg/kg, twice a day for 5 days) and human Fc fragments were used as a negative control for the experiment. On the fourth day after infection, lung tissues from selected mice were taken, homogenized, and tested for virus titers in lung. Body weight change and survival of mice were monitored daily for 15 days after infection.

H3N2 Model: Female BALB/c mice aged 6-8 weeks were infected using a lethal dose of influenza virus strain H3N2 A/HongKong/1/68. On day 0, inoculation with nasal drops was performed at a dose of 1×LD₉₀. Intravenous administration was performed 4 hours prior to inoculation. Oral Oseltamivir administration was used as a positive control for the experiment (Administered 8 hours after infection, 50 mg/kg, twice a day for 5 days) and human Fc fragments were used as a negative control for the experiment. On the fourth day after infection, lung tissues from selected mice were taken, homogenized, and tested for virus titers in lung. Body weight change and survival of mice were monitored daily for 15 days after infection.

B Model: Female BALB/c mice aged 6-8 weeks were infected using a lethal dose of influenza virus strain B/Malaysia/2506/04. On day 0, inoculation with nasal drops was performed at a dose of 1×LD₉₀. Intravenous administration was performed 4 hours prior to inoculation. Oral Oseltamivir administration was used as a positive control for the experiment (Administered 8 hours after infection, 50 mg/kg, twice a day for 5 days) and human Fc fragments were used as a negative control for the experiment. On the fourth day after infection, lung tissues from selected mice were taken, homogenized, and tested for virus titers in lung. Body weight change and survival of mice were monitored daily for 15 days after infection.

### Example 43: Mouse Therapeutic Model for Lethal Dose Influenza Virus

H1N1 Model: Female BALB/c mice aged 6-8 weeks were infected using a lethal dose of influenza virus strain H1N1 A/Texas/36/91. On day 0, inoculation with nasal drops was performed at a dose of 1×LD₉₀. Intravenous administration was performed 4 hours prior to inoculation. Oral Oseltamivir administration was used as a positive control for the experiment (Administered 8 hours after infection, 50 mg/kg, twice a day for 5 days) and human Fc fragments were used as a negative control for the experiment. On the fourth day after infection, lung tissues from selected mice were taken, homogenized, and tested for virus titers in lung. Body weight change and survival of mice were monitored daily for 15 days after infection.

H3N2 Model: Female BALB/c mice aged 6-8 weeks were infected using a lethal dose of influenza virus strain H3N2 A/HongKong/1/68. On day 0, inoculation with nasal drops was performed at a dose of 1×LD₉₀. Intravenous administration was performed 4 hours prior to inoculation. Oral Oseltamivir administration was used as a positive control for the experiment (Administered 8 hours after infection, 50 mg/kg, twice a day for 5 days) and human Fc fragments were used as a negative control for the experiment. On the fourth day after infection, lung tissues from selected mice were taken, homogenized, and tested for virus titers in lung. Body weight change and survival of mice were monitored daily for 15 days after infection.

B Model: Female BALB/c mice aged 6-8 weeks were infected using a lethal dose of influenza virus strain B/Malaysia/2506/04. On day 0, inoculation with nasal drops was performed at a dose of 1×LD₉₀. Intravenous administration was performed 4 hours prior to inoculation. Oral Oseltamivir administration was used as a positive control for the experiment (Administered 8 hours after infection, 50 mg/kg, twice a day for 5 days) and human Fc fragments were used as a negative control for the experiment. On the fourth day after infection, lung tissues from selected mice were taken, homogenized, and tested for virus titers in lung. Body weight change and survival of mice were monitored daily for 15 days after infection.

### Example 44: Activity of Compounds to be Tested in Fatal Influenza Virus Infected Immunodeficient Mouse Model

Immunodeficient (SCID) mice aged 6-8 weeks were infected using a lethal dose of influenza virus strain A/Puerto Rico/08/1934. On day 0, inoculation with nasal drops was performed at a dose of 3×LD₉₅. Intravenous administration (a dosage of 0.3, 1.0, 3.0 mg/kg) was performed prior to inoculation. Oral Oseltamivir administration was used as a positive control for the experiment (Administered 8 hours after infection, 50 mg/kg, twice a day for 5 days) and human Fc fragments were used as a negative control for the experiment. On the fourth day after infection, lung tissues from selected mice were taken, homogenized, and tested for virus titers in lung. Body weight change and survival of mice were monitored daily for 35 days after infection.

### Example 45. In-Vivo Toxicity of the Compound to be Tested

14-day rat dose-ranging study was performed. On days 0 and 7, rats were injected intravenously with 5 mpk, 20 mpk, or 50 mpk of the compound to be tested. Changes in body weight, organ weights, and food intake for rats were monitored and compared to that of the control group. Blood exposure of the compound to be tested is measured by sandwich ELISA as described in Example 39 above.

### Example 46. In-Vivo Efficacy of the Compound to be Tested Against the Oseltamivir-Resistant Influenza Virus Strain

Influenza strain H1N1/A/Perth/261/2009 passaged by mouse is oseltamivir resistant and contains the H275Y mutation. At day 0, inoculation with nasal drops was performed at a dose of 1×LD₉₀. Intravenous administration was performed 4 hours prior to inoculation. Oral Oseltamivir administration was used as a positive control for the experiment (Administered 8 hours after infection, 50 mg/kg, twice a day for 5 days) and human Fc fragments were used as a negative control for the experiment. Body weight change and survival of mice were monitored daily for 15 days after infection.

### Example 47: Fatal Influenza Virus Infected Mouse Model with Delayed Treatment Time

The compounds to be tested were injected into mice via intravenous administration at various time after infection (2 hrs before infection, 2 hrs after infection, 24 hrs, 48 hrs, 72 hrs, 96 hrs after infection). The time of infection was marked as day 0. All mice were inoculated with 2×LD₉₅ of H1N1 A/Texas/36/91 by nasal drip. Oral Oseltamivir was used as a positive control for the experiment (Administered 8 hours after infection, 50 mg/kg, twice a day for 5 days) and human Fc fragments were used as a negative control for the experiment. Body weight change and survival of mice were monitored daily for 15 days after infection.

### Example 48: Combined Efficacy of Compounds and Other Antiviral Drugs

Influenza virus: the compound to be tested is diluted in a 10-fold series at a concentration range of 0.001-100 nM and another anti-influenza virus compound is a known clinical or approved drug such as baloxivir, pimodivir, oseltamivir, zanamivir, peramivir, laninamivir, amantadine, MEDI8852 or rimantadine, which were cross-mixed in the concentration range of 1-1000 nM and then added to MDCK cells overnight. The Influenza virus H1N1/A/PR8/34 with an infection index of 0.001-1 was added at the next day. After 4 days of infection, the cells were stained with crystal violet dye and the absorbance at 595 nm was read. The experimental data of drug synergistic effect was analyzed by MacSynergy.

### Example 49: 28-day Rat Pharmacokinetic Study in Intravenous or Subcutaneous Administration

Rats are administered 5 mg/kg of the compound to be tested intravenously or subcutaneously (5 ml/kg dose volume). Blood (EDTA anticoagulated tubes) is taken at various time points (24 hours, 3 days, 7 days, 14 days, 28 days) after administration. After centrifugation (2000×g, 10 min) of the whole blood, plasma was taken to analyze the concentration of the compound to be tested. Blood exposure of the compound to be tested was assayed using the ELISA as described in Example 39 above.

### Example 50: 28-Day Non-Human Primate Pharmacokinetic Study (Intravenous Administration)

Macaca fascicularis aged 4.5-8 years weighing 2.5-6.5 kg were administered 5 mg/kg or 20 mg/kg of the compound to be tested intravenously (5 ml/kg dose volume). All animals were subjected to IACUC standard experimental conditions. Blood (EDTA anticoagulated tubes) was taken at different time points (24 h, 3 days, 7 days, 14 days, 28 days) after administration. After centrifugation (2000×g, 10 min) of whole blood, plasma was taken to analyze the concentration of the compound to be measured. Blood exposure of the compound to be tested was assayed using the ELISA as described in Example 39 above.

### Example 51: Pharmacokinetic Study on the Distribution of Lung Tissue in Mice (Intravenous Administration)

The CD-1 mice aged 6 weeks were intravenously injected with 10 mg/kg (5 ml/kg dose volume) via tail. All animals were under standard IACUC experimental conditions. At various time after administration, mice were euthanized, and blood (EDTA anticoagulated tubes) and lungs were taken. After centrifugation (2000×g, 10 min) of whole blood, plasma was taken to analyze the concentration of the compound to be measured. Lungs were placed in a centrifuge tube, weighed, homogenized to 100 μL that was adjusted to 1 mL, placed on ice for 20 minutes and mixed. The homogenate was centrifuged (8000×g for 10 minutes) and the supernatant was taken to analyze the concentration of the compound to be measured. The ELISA assay as described in Example 39 above was performed.

### Example 52: Single-Dose In Vivo Toxicity Study in Rats

Sprague-Dawley rats were subcutaneously administered at a dose of 100 mg/kg, 200 mg/kg, or 400 mg/kg, in a 5 ml/kg administration volume. After the drug administration, the health status, feeding, body weight changes, and physiological indicators of the animals were observed for 15 days. On the 15th day, blood was taken to assay the blood biochemical indicators and various histopathological examinations.

### Example 53: In Vitro Stability

Metabolic stability studies in freshly extracted plasma and liver microsomes from mice and human were performed. After co-incubating the compounds to be tested with the plasma or liver microsomes at 37° C. for 24 hours, the changes in DAR of the compounds to be tested were detected by MALDI-TOF mass spectrometry. This method identifies the structural parts of the compounds to be tested that have poor metabolic stability.

### Example 54: In Vitro FcγR Binding Activity Studies

Recombinant FcγR (I, IIA, IIC, III) protein (1 g/mL) was encapsulated in an ELISA plate overnight and was blocked with 2% bovine serum PBS for 1 hour at room temperature on the next day. The compounds to be tested (0.01-1000 nM) that have been diluted in serial were added and incubated for 1 hour at room temperature. The horseradish peroxidase-labeled secondary antibody against human IgG-Fc was used for assay in conjunction with color development performed by adding TMB substrate for 20 min and addition of an equal volume of reaction termination solution. The absorbance value was read at OD450 nm.

### Example 55: Antibody-Dependent Cytotoxicity

Influenza virus AVC compounds: MDCK cells were infected with A/PR/8/1934 (H1N1), A/HK/1/1968 (H3N2) or B/Malaysia/2506/2004 (Victoria) that have an infection factor in the range of 0.001-10, for 18-24 hrs at 37° C. with 5% CO2. Then, the compounds to be tested were added and ADCC activity was assayed using the PROMEGA kit. Gedivumab (Genentech) was used as a positive control and Fc-N297A was used as a negative control.

### Example 56: Antibody-Dependent Cell Phagocytosis

Influenza virus AVC compounds: MDCK cells were infected with A/PR/8/1934 (H1N1), A/HK/1/1968 (H3N2) or B/Malaysia/2506/2004 (Victoria) that have an infection factor in the range of 0.001-10, for 18-24 hrs at 37° C. with 5% CO2. Then, the compounds to be tested were added and ADCP activity was assayed using the PROMEGA kit. Gedivumab (Genentech) was used as a positive control and Fc-N297A was used as a negative control.

### Example 57: Prevention of Secondary Infections Caused by Viral Infections

Influenza virus infection induced methicillin-resistant Staphylococcus aureus (MRSA) infection model: 6-8 week old BALB/c mice were intranasally inoculated (sublethal dose) with H1N1 A/CA/07/2009pdm influenza virus. The compound to be tested was injected intravenously 2 hours after infection (0.3-3 mg/kg). On day 6 after infection, mice were intranasally inoculated with a sublethal dose (5×10⁷ CFU) of methicillin-resistant Staphylococcus aureus (MRSA) TCH1516. 24 hrs later, lung tissues were taken for bacterial load testing. Lung tissues were homogenized in PBS solution with 1 mm diameter of glass beads and then diluted in a 10-fold series, coated on LA plates and incubated for 1 day at 37° C. CFU were finally converted to bacterial load per g of lung tissue weight. Body weight change and survival of mice were monitored daily for 14 days after infection.

Influenza virus infection induced Streptococcus pneumoniae infection model: 6-8 week old BALB/c mice were intranasally inoculated (sublethal dose) with H1N1 A/CA/07/2009pdm influenza virus. The compound to be tested was injected intravenously 2 hours after infection (0.3-3 mg/kg). On day 6 after infection, mice were inoculated intranasally with a sublethal dose (1×10⁵ CFU) of Streptococcus pneumoniae 6301. 24 hrs later, lung tissues were taken for bacterial load testing. Lung tissues were homogenized in PBS solution with 1 mm diameter of glass beads and then diluted in a 10-fold series, coated on LA plates and incubated for 1 day at 37° C. CFU were finally converted to bacterial load per g of lung tissue weight. Body weight change and survival of mice were monitored daily for 14 days after infection.

### Example 58. Human FcRn Transgenic Mouse Viral Infection Model

Influenza virus infection: 6-8 week old female B6.Cg-Fcgrttml Dcr Tg(GCGRT)32Dcr/DcrJ mice (Jackson Labs #014565) express the human fetal FcRn receptor. Compounds to be tested having YTE Fc enables to show their extended half-life in this model. Intranasal inoculation with 3×LD95 lethal dose of H1N1/A/CA/07/2009 influenza virus were performed. Mice were administered the compound to be tested intravenously (0.01, 0.03, 0.1, 0.3, 1.0 mg/kg, 5 ml/kg dose volume) at 7 days prior to infection. Oral Oseltamivir was used as a positive control for the experiment (Administered 8 hours after infection, 50 mg/kg, twice a day for 5 days) and human Fc fragments were used as a negative control for the experiment. Body weight change and survival of mice were monitored daily for 15 days after infection. Compared to compounds without YTE Fc, compounds with YTE was more effective in preventing viral infection and animal death at the same dose.

1. A conjugate of formula I-1, or a pharmaceutically acceptable salt, ester, isomer, solvate, prodrug or isotopically labeled derivative thereof, 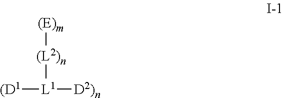
wherein,
m is 1 or 2;
n is an integer from 1 to 20, preferably an integer from 1 to 10, e.g., 1, 2, 3, 4, 5, 6, 7, 8, 9 or 10; and two n's are identical;
each of D1 and D2 is an anti-influenza virus small molecule drug, and is independently selected from a compound having an anti-influenza activity as shown in formulae D-1-1 to D-1-8: 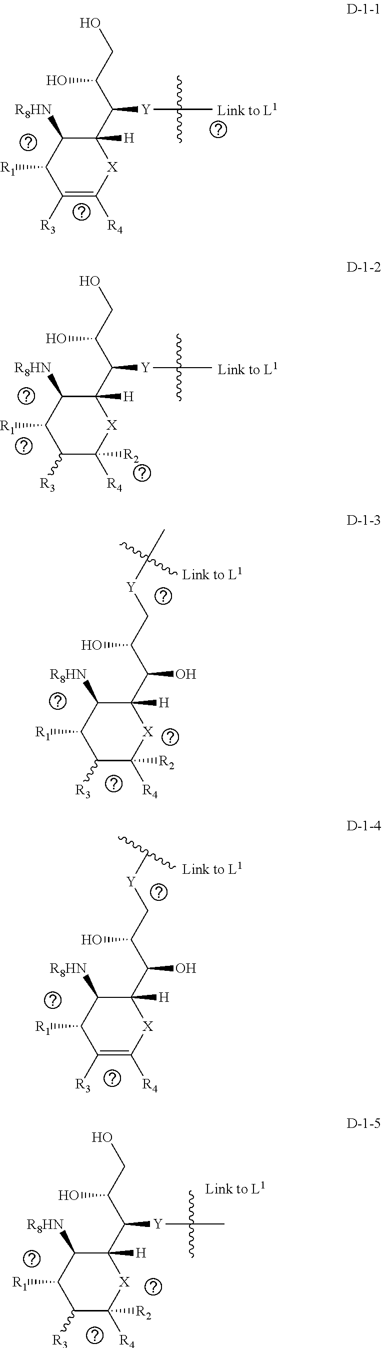 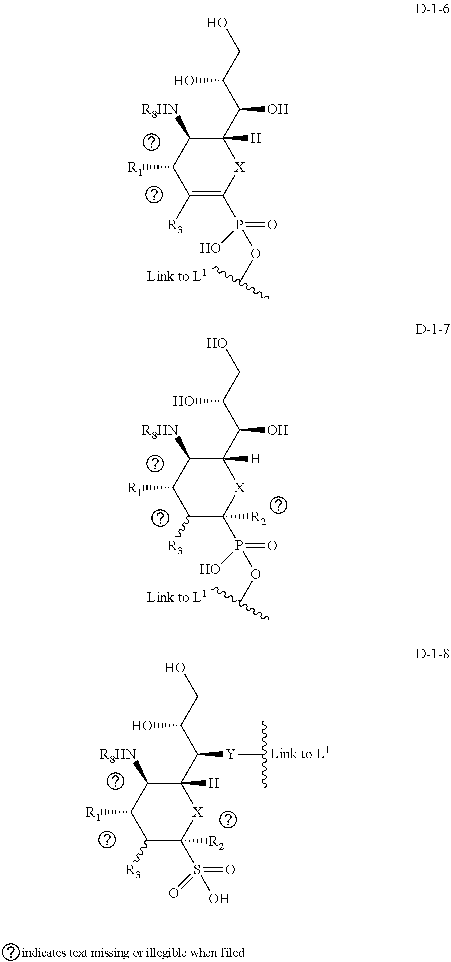
R₁ is selected from OH, NH₂, —NH(═NH)NH₂ and —NHC(═NH)NHR₆;
R₂ and R₃ are each independently selected from H, OH, F, Cl and Br;
R₄ is selected from —COOH, —P(═O)(OH)₂ and —SO₃H;
R₅ is selected from —COC₁₋₆ alkyl, —COC₁₋₆ haloalkyl, —SO₂C₁₋₆ alkyl, and —SO₂C₁₋₆ haloalkyl; preferably selected from —COCH₃, —COCF₃, and —SO₂CH₃;
X is selected from O and S;
R₆ is selected from the following groups: 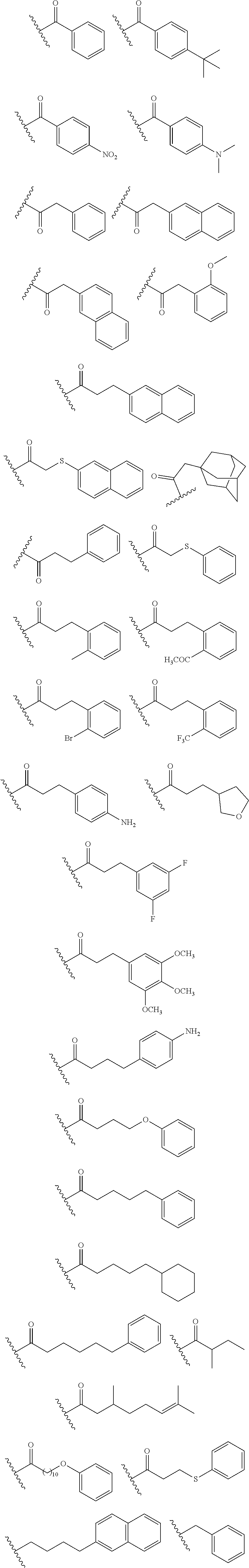
Y is selected from the following groups, wherein “(attached to L¹)” specifies the end of the group Y attached to the end of L¹, wherein N denotes an atom within the ring when drawn inside the ring: 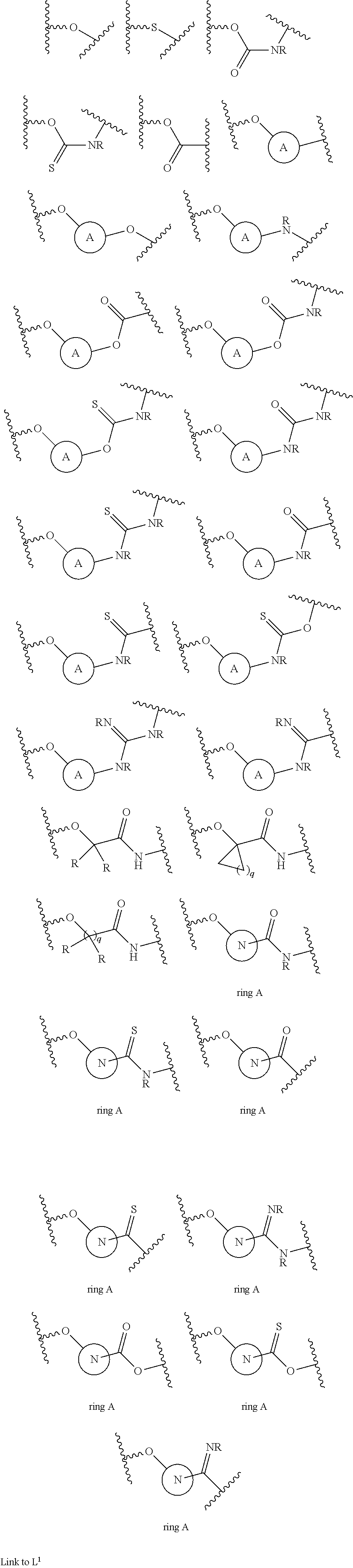
ring A is selected from C₃-C₂₀ cycloalkyl, substituted C₃-C₂₀ cycloalkyl, C₃-C₂₀ cycloalkenyl, substituted C₃-C₂₀ cycloalkenyl, C₆-C₁₅ aryl, 3- to 20-membered heterocycloalkyl, substituted 3- to 20-membered heterocycloalkyl, substituted C₆-C₁₅ aryl and substituted 5- to 15-membered heteroaryl;
R groups are each independently selected from H, deuterium, optionally substituted C₁-C₂₀ alkyl, optionally substituted C₂-C₂₀ streptenyl, optionally substituted C₃-C₂₀ cycloalkyl, optionally substituted 3- to 20-membered heterocycloalkyl, optionally substituted C₆-C₁₅ aryl, and optionally substituted 5- to 15-membered heteroaryl;
q is 0, 1, 2, 3, 4, 5, 6, 7, 8, 9 or 10;
L¹ is a linker attached to the drug (D¹ or D²) and L², and includes but not limited to following structures:
wherein bonds at left and right ends of the following structures are attached to an oxygen atom of the drug and a bond marked by the wavy line in the middle is attached to L², 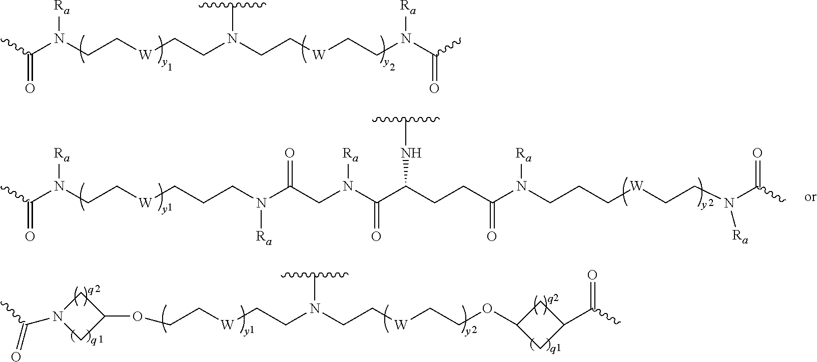
wherein:
W is selected from O, S, NR^{b}, CH₂ or absent;
R^{a} and R^{b} are each independently selected from H, optionally substituted C₁-C₂₀ alkyl, optionally substituted C₂-C₂₀ alkenyl;
y¹ and y² are each independently 0, 1, 2, 3, 4, 5 or 6;
q¹ and q² are each independently selected from 1, 2, 3, 4, 5 and 6;
or
L¹ is selected from following structures: 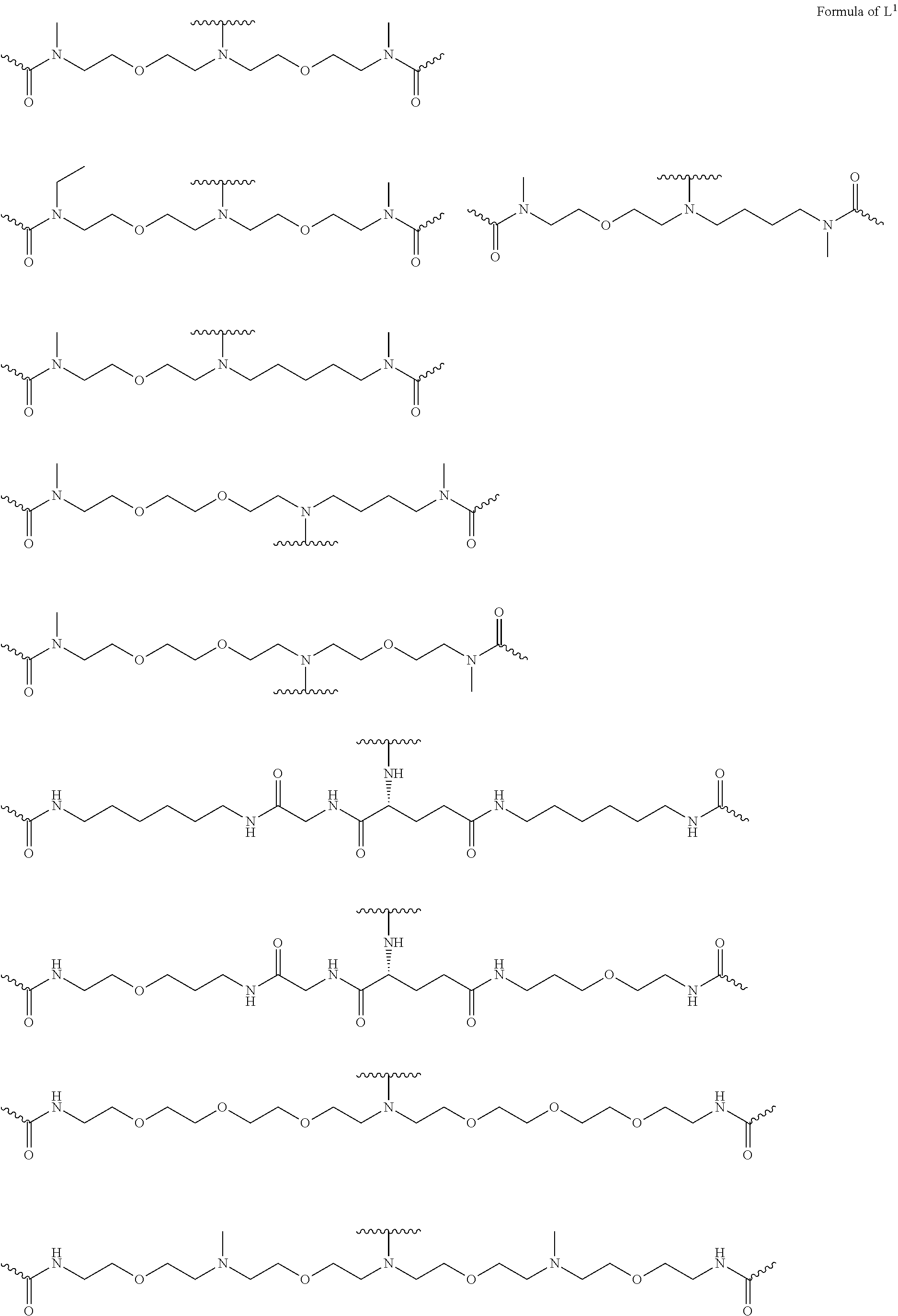 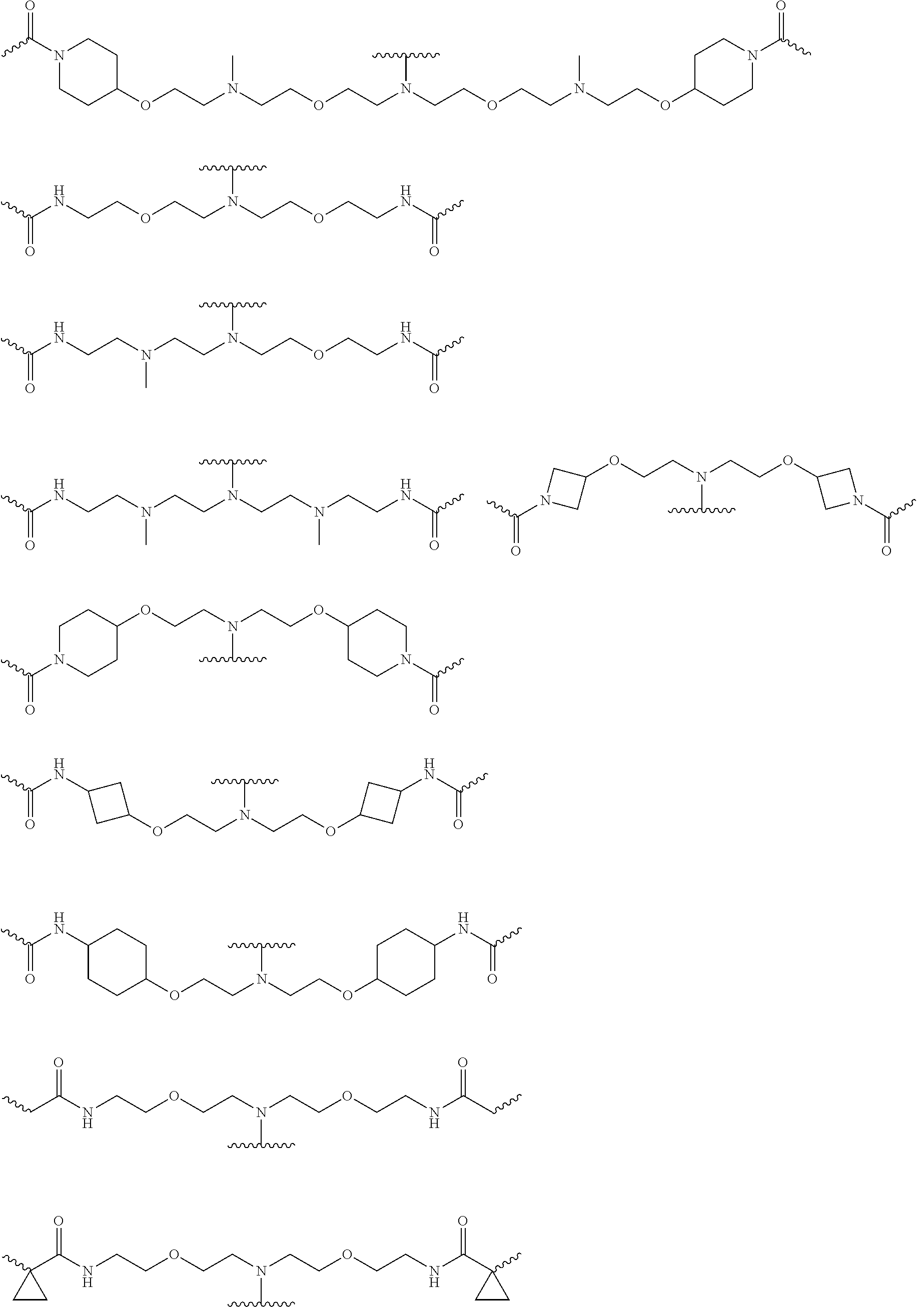 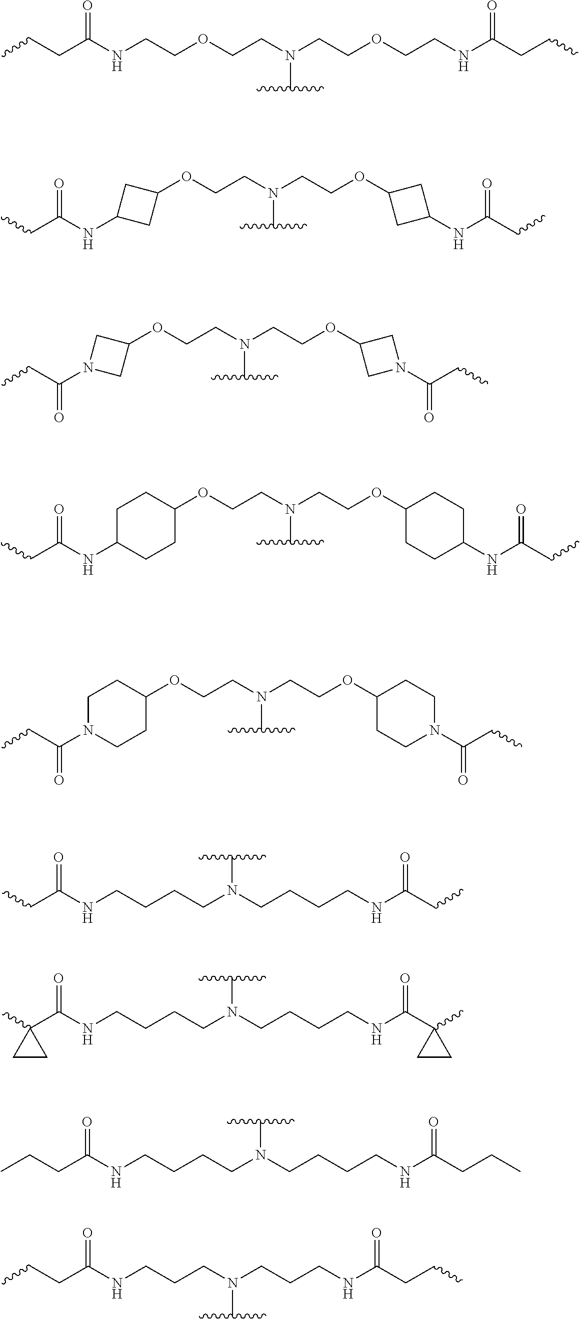 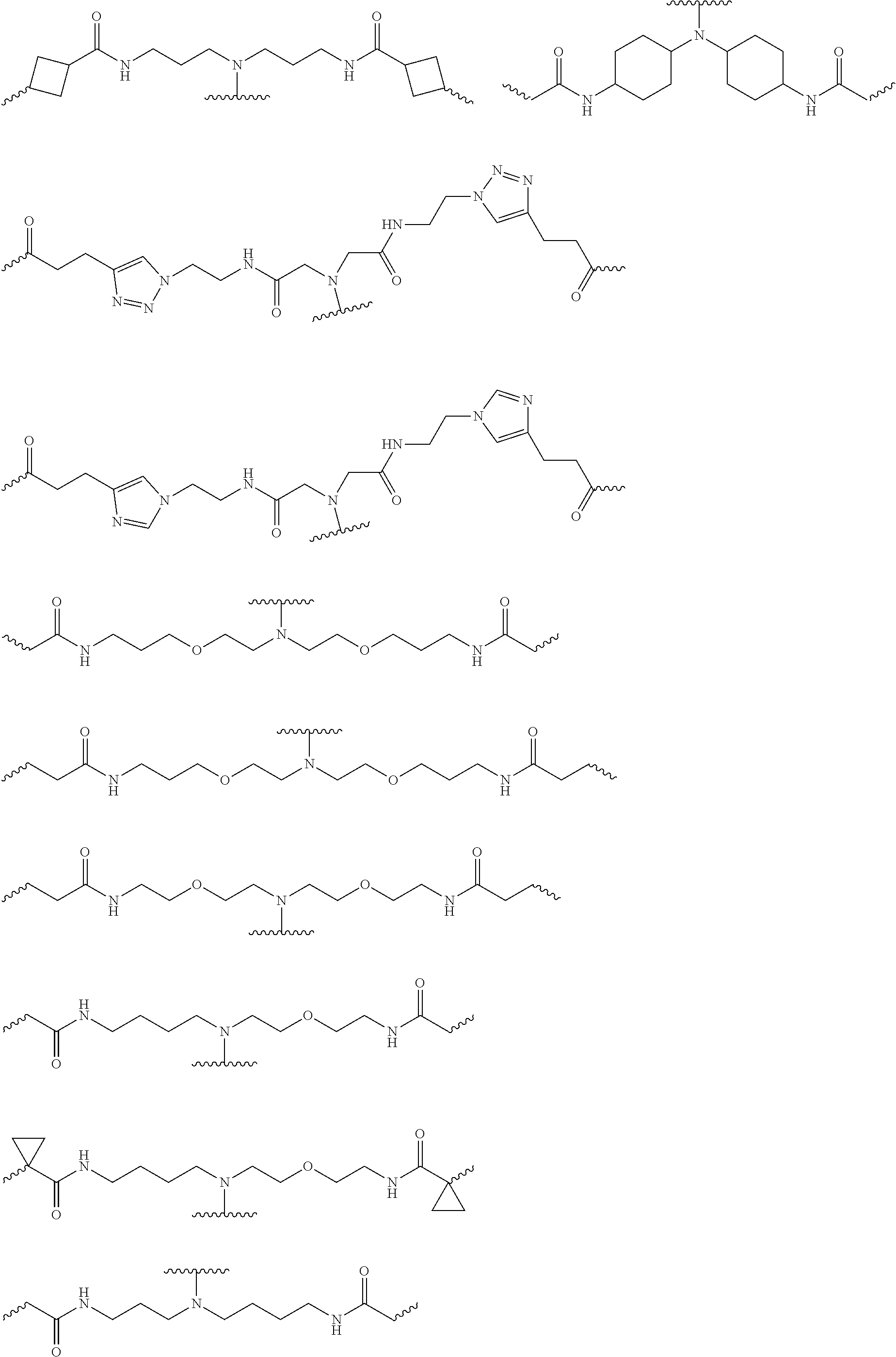 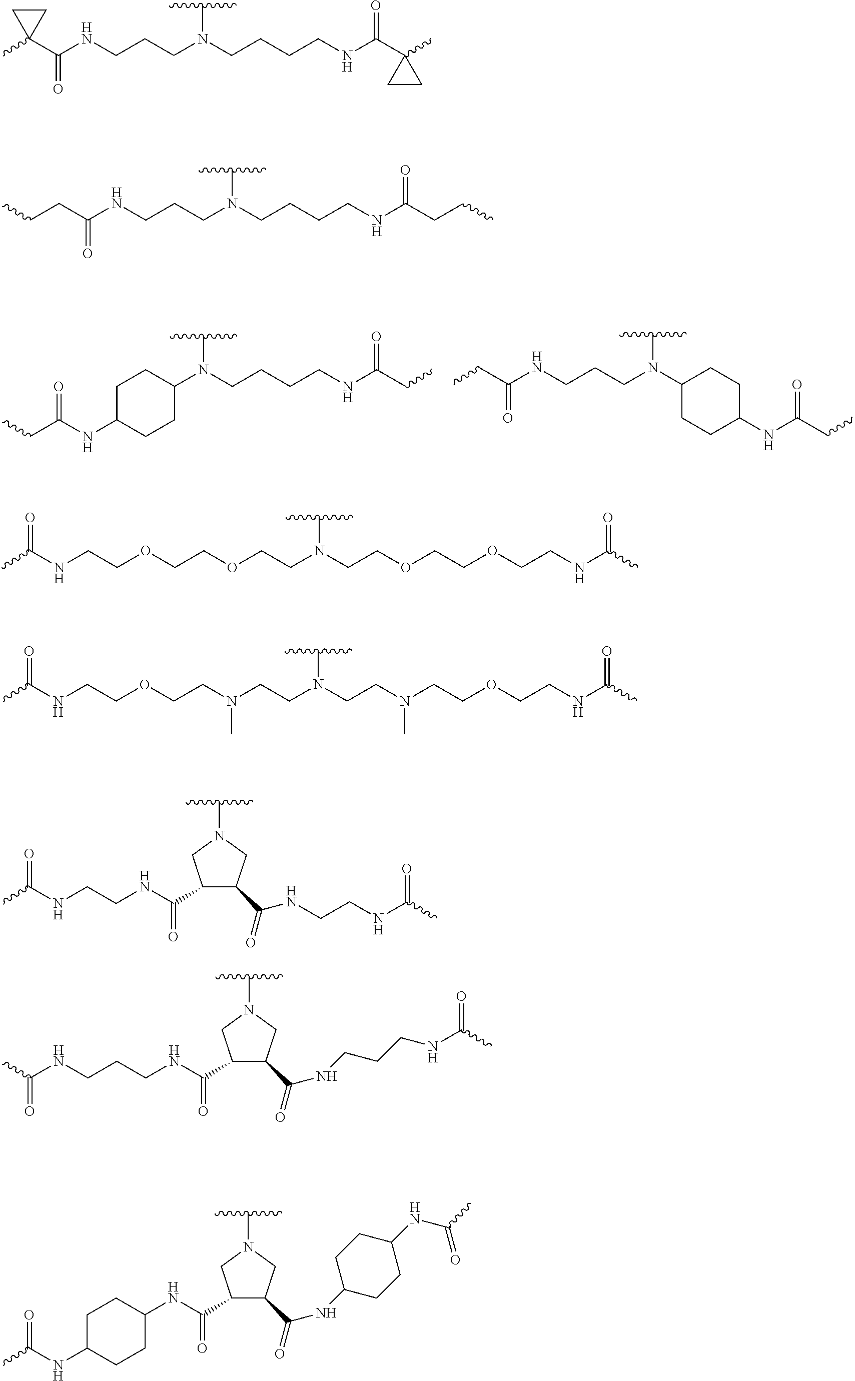 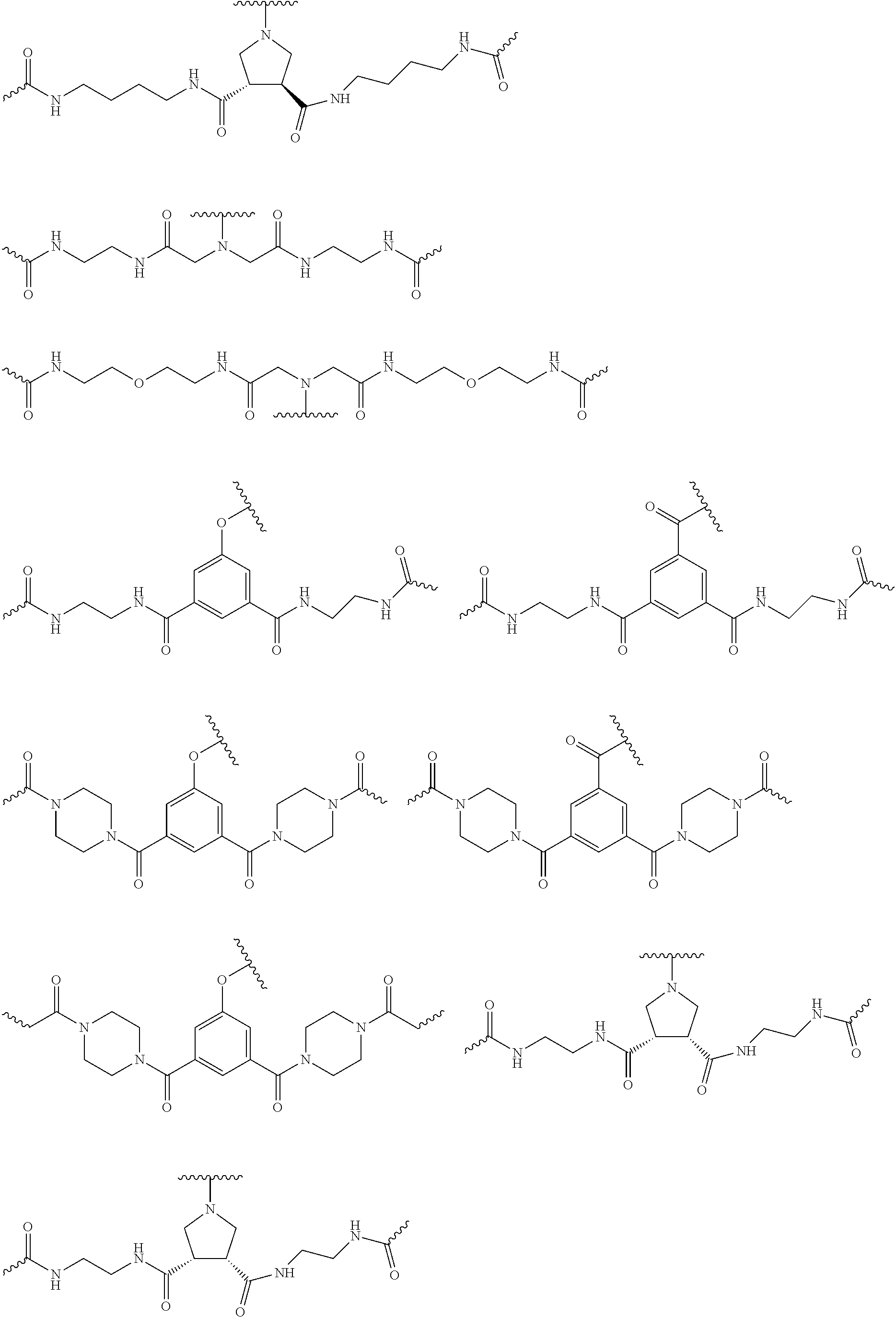 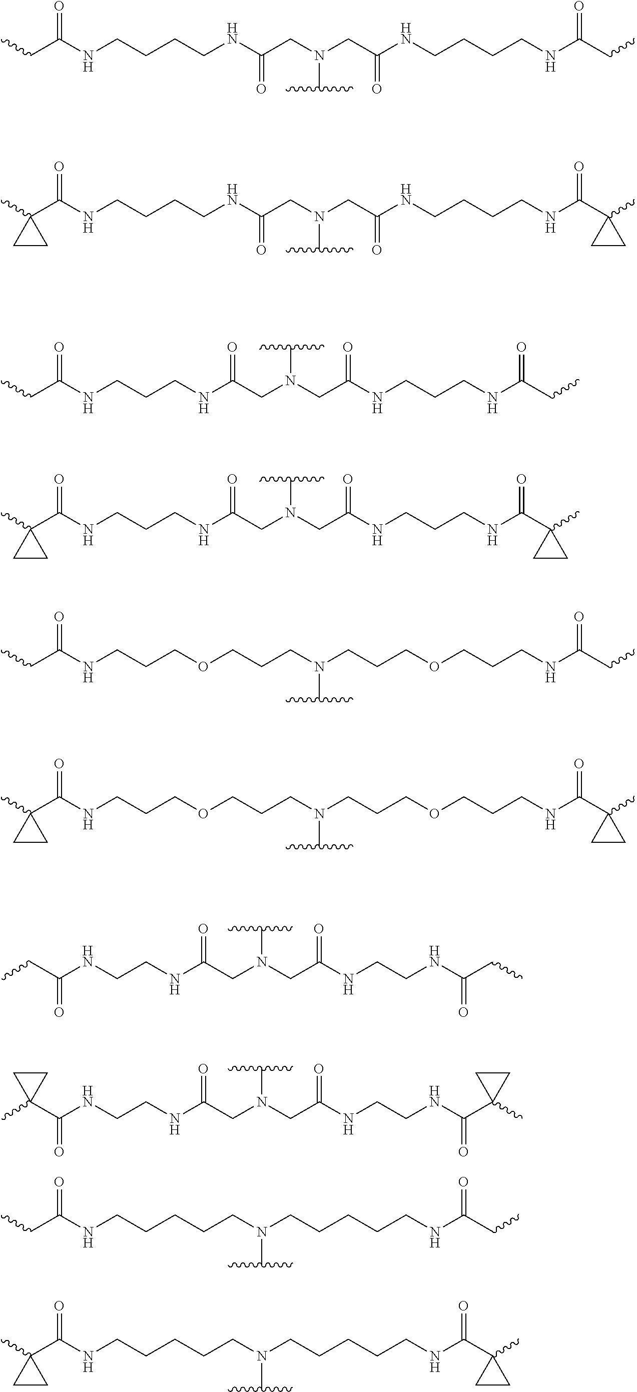 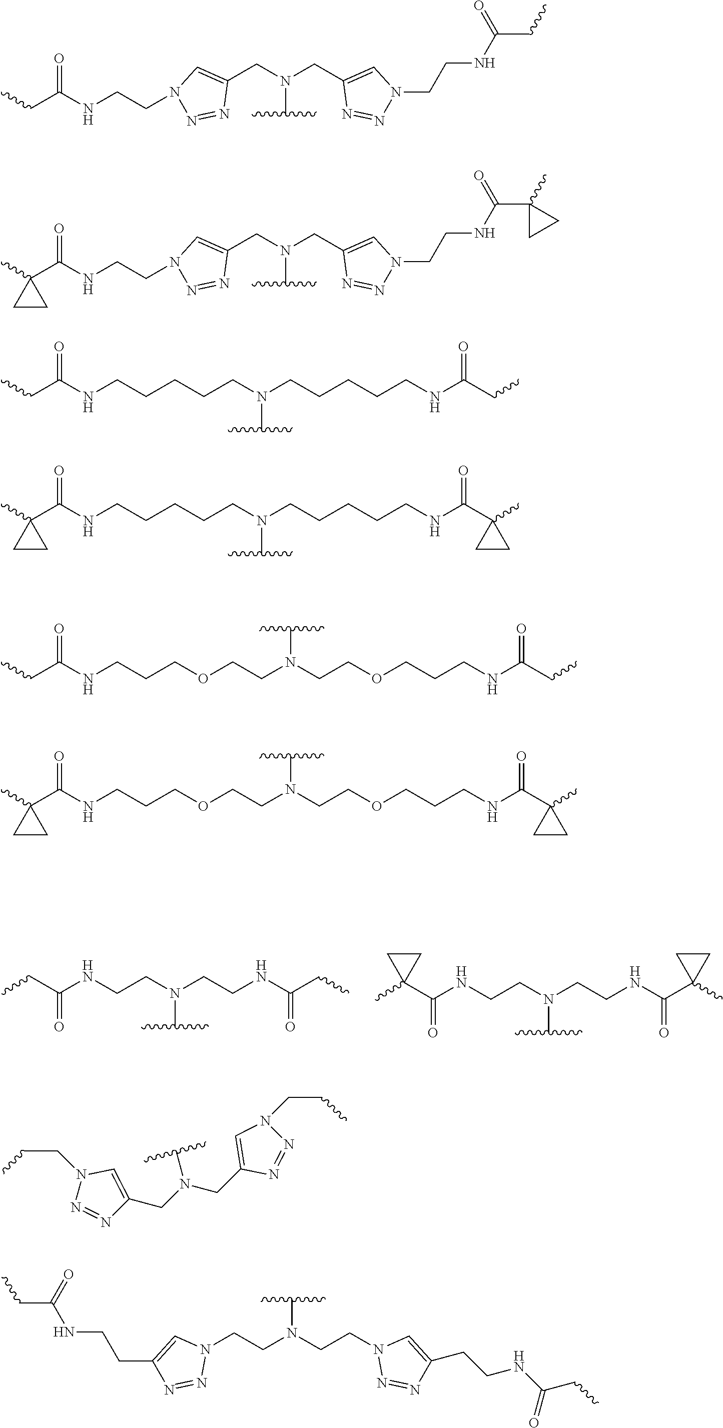 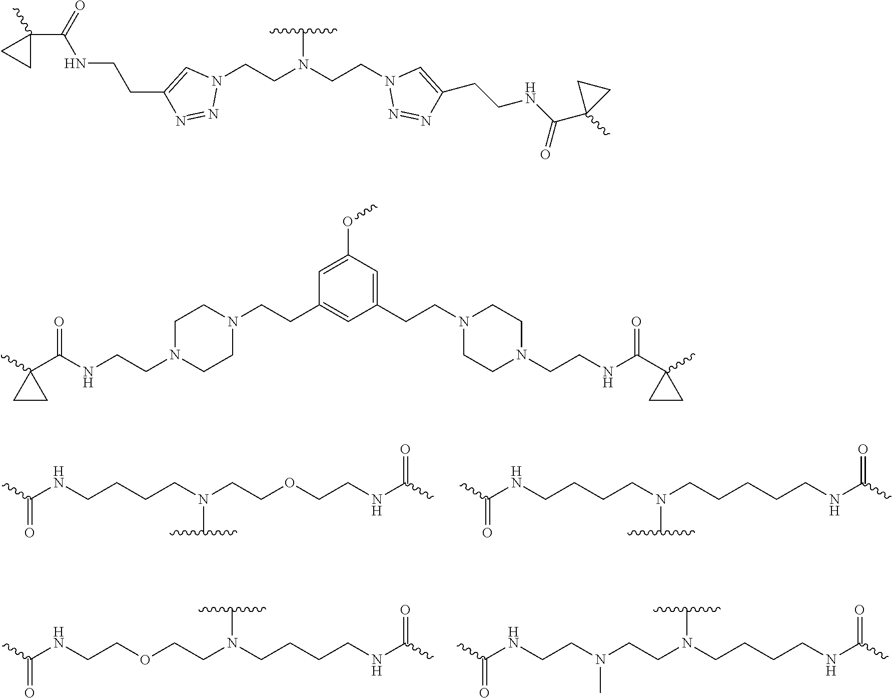 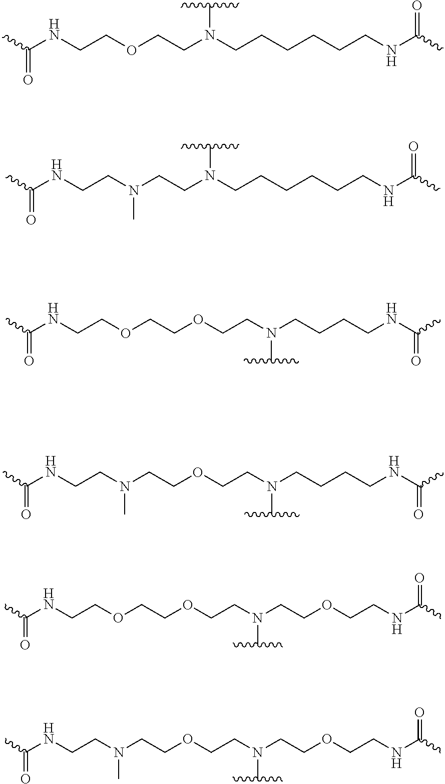 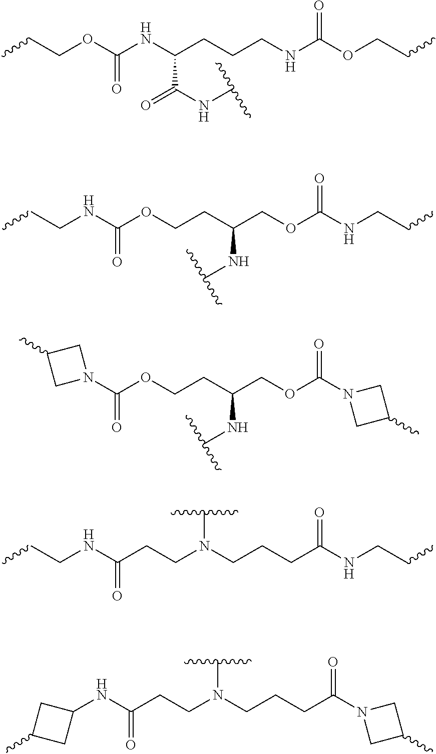
L² is a linker attaching L¹ and E (Fc monomer, Fc structural domain, Fc-binding peptide, albumin, or albumin-linking peptide) and having a structure sleeted from formula L2-1 to L2-15, wherein (L¹) and (E) denote ends of L² connected to L¹ and E, respectively:

| L² structure No. | Structure |
|---|---|
| L2-1 | 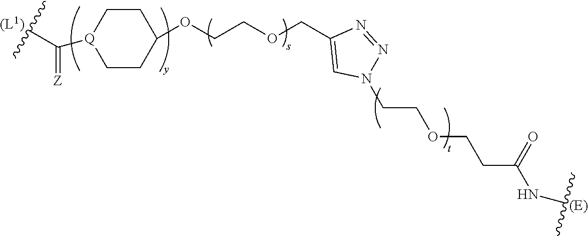 |
| L2-2 | 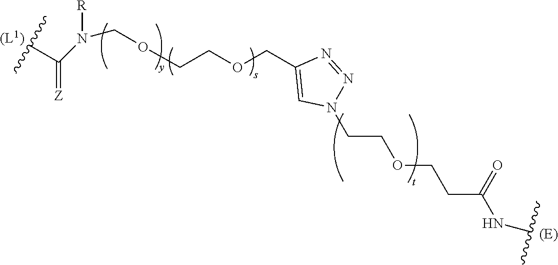 |
| L2-3 | 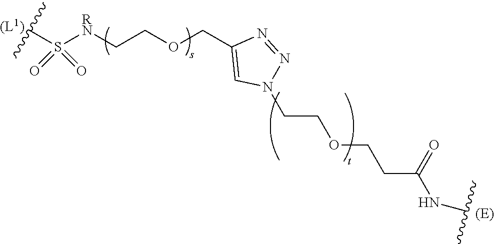 |
| L2-4 | 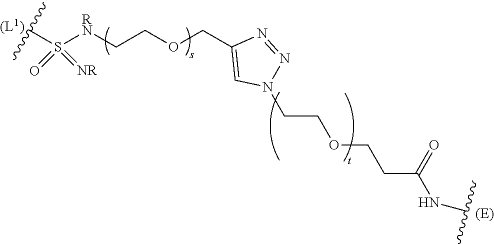 |
| L2-5 | 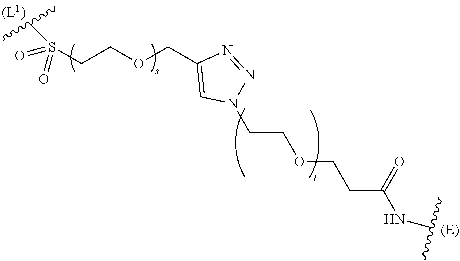 |
| L2-6 | 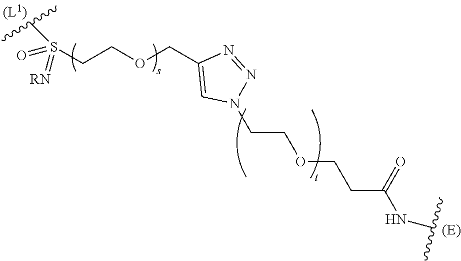 |
| L2-7 | 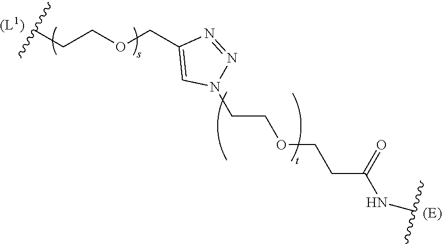 |
| L2-8 | 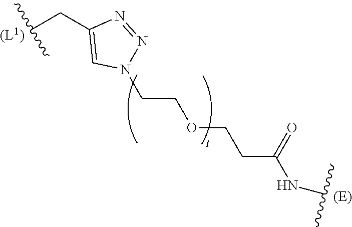 |
| L2-9 | 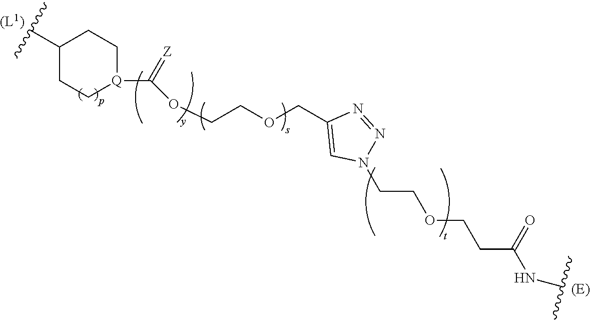 |
| L2-10 | 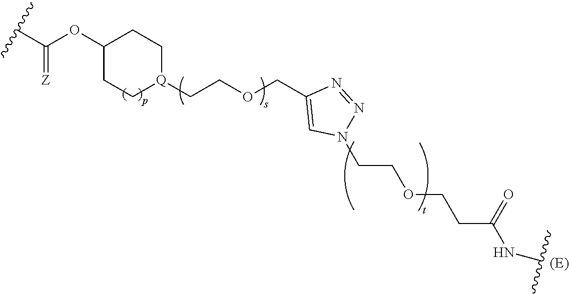 |
| L2-11 | 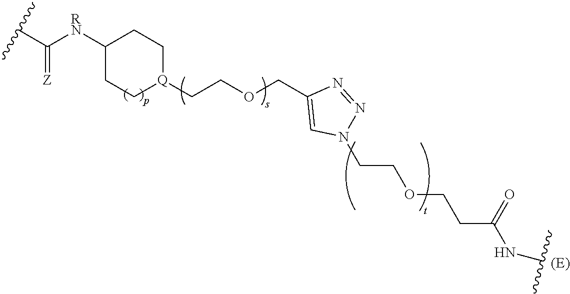 |
| L2-12 | 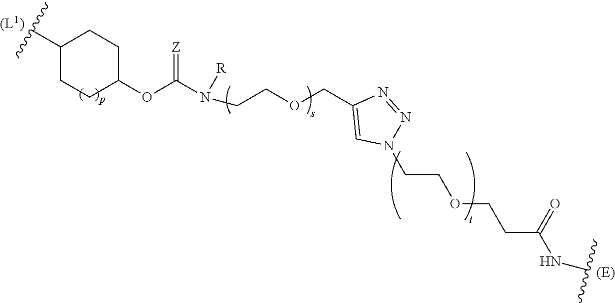 |
| L2-13 | 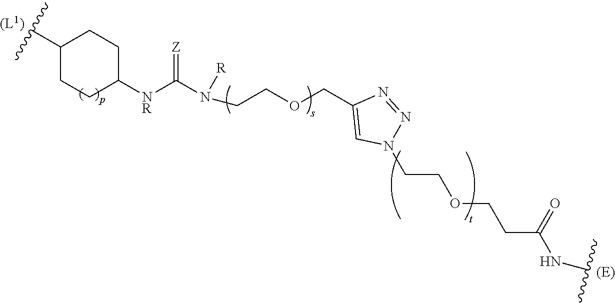 |
| L2-14 | 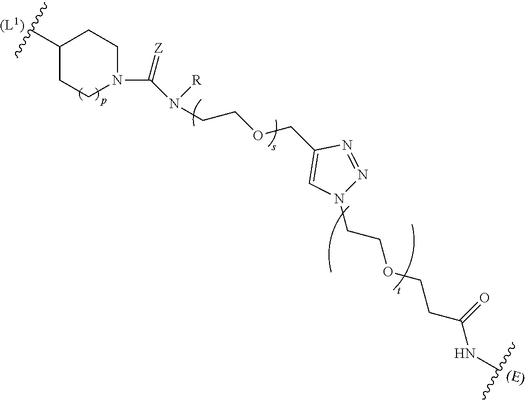 |
| L2-15 | 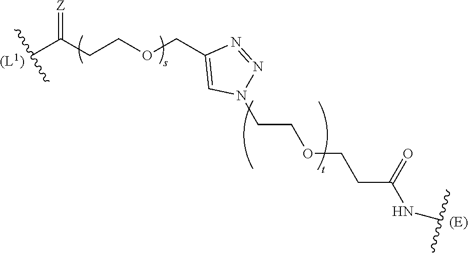 |

wherein,
Z is NR, S or O;
R are each independently selected from hydrogen, deuterium, optionally substituted C₁-C₂₀ alkyl, optionally substituted C₂-C₂₀ chain alkenyl, optionally substituted C₃-C₂₀ cycloalkyl, optionally substituted 3- to 20-membered heterocycloalkyl, optionally substituted C₆-C₁₅ aryl and optionally substituted 5- to 15-membered heteroaryl;
s and t are integers from 1 to 20; such as 1, 2, 3, 4, 5, 6, 7, 8, 9, 10, 11 or 12;
y is 0 or 1;
Q is N or CH;
p is 0, 1, 2, 3, 4, 5, 6, 7 or 8;
E is an antibody or an antibody fragment thereof, an Fc structural domain monomer, an Fc structural domain, an Fc-binding peptide, an albumin or an albumin-linking peptide, and comprises an amino acid sequence of any one of SEQ ID Nos. 1-68 or an amino acid sequence that is at least 95% identical to any one of SEQ ID Nos. 1-68.

2. The conjugate of claim 1, or a pharmaceutically acceptable salt, ester, isomer, solvate, prodrug or isotopically labeled derivative thereof, 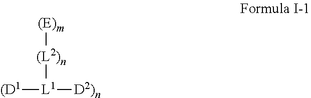
wherein
m is 1 or 2;
n is an integer from 1 to 10;
each of D¹ and D² is independently selected from the compound having an anti-influenza activity as shown in the formulae D-1-1 to D-1-8;
D¹ and D² are covalently attached to L¹ that is covalently attached to L², and L² is covalently attached to E;
E is selected from the Fc structural domain monomer, the Fc structural domain, the Fc-binding peptide, the albumin or the albumin-linking peptide.

3. The conjugate of claim 1, or a pharmaceutically acceptable salt, ester, isomer, solvate, prodrug or isotopically labeled derivative thereof, wherein the conjugate has a structure of formula I-1-1 to I-1-8:

| Conjugate No. | Structure |
|---|---|
| I-1-1 | 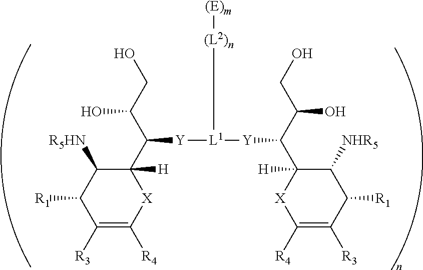 |
| I-1-2 | 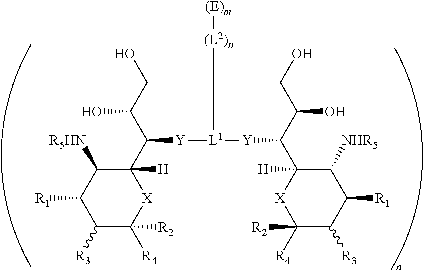 |
| I-1-3 | 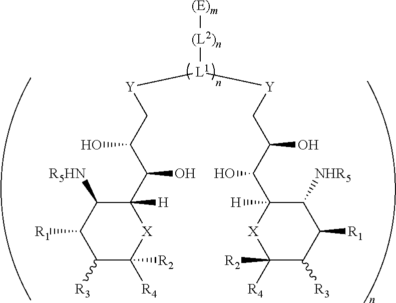 |
| I-1-4 | 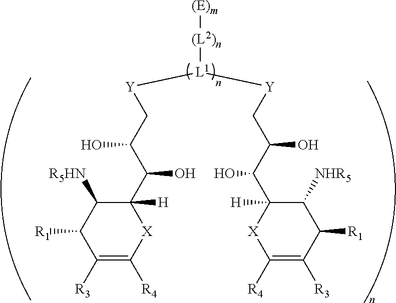 |
| I-1-5 | 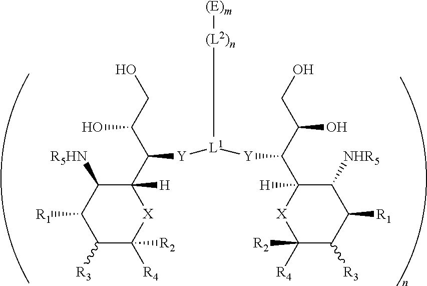 |
| I-1-6 | 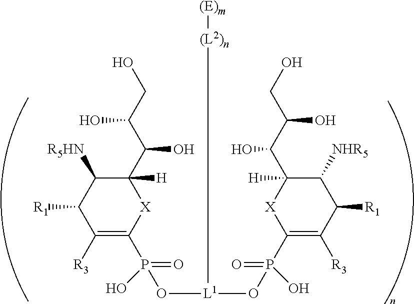 |
| I-1-7 | 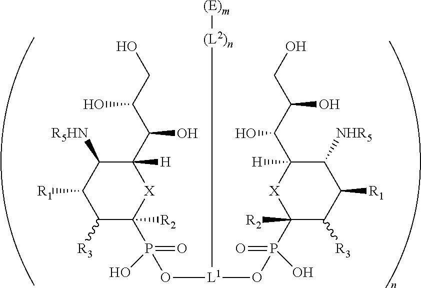 |
| I-1-8 | 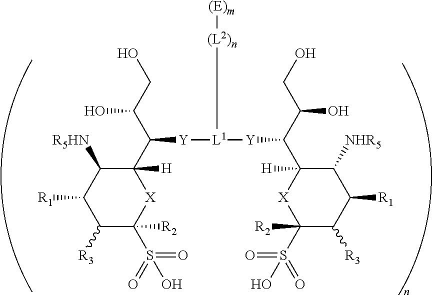 |

wherein each variable is as defined in claim 1.

4. The conjugate of claim 1, or a pharmaceutically acceptable salt, ester, isomer, solvate, prodrug or isotopically labeled derivative thereof, wherein the conjugate has a structure of formula I-1-A to I-1-D:

| No. | Structure |
|---|---|
| I-1-A | 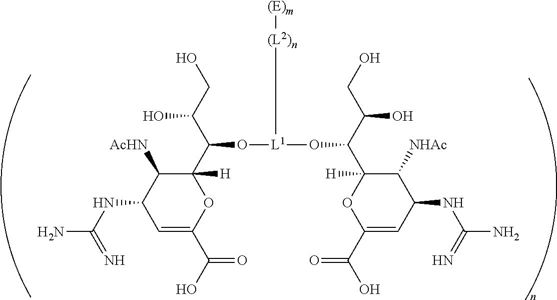 |
| I-1-B | 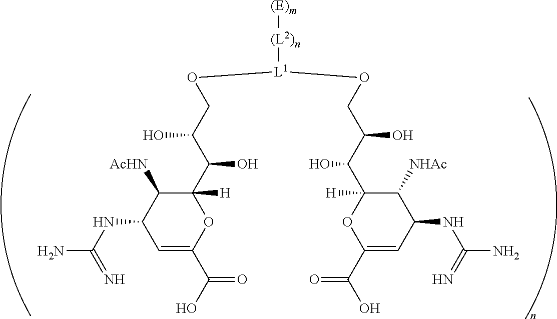 |
| I-1-C | 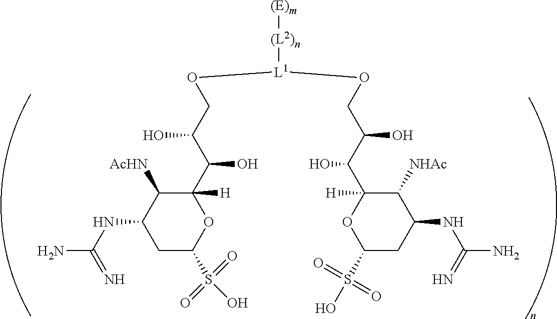 |
| I-1-D | 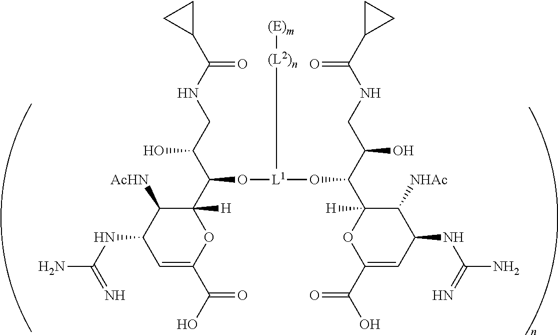 |

wherein each variable is as defined in claim 1.

5. The conjugate of claim 1, or a pharmaceutically acceptable salt, ester, isomer, solvate, prodrug or isotopically labeled derivative thereof, wherein the conjugate has a structure of formula C-1 to C-115:

| Conju- | |
|---|---|
| gate | |
| No. | Conjugate Structure |
| C-1 | 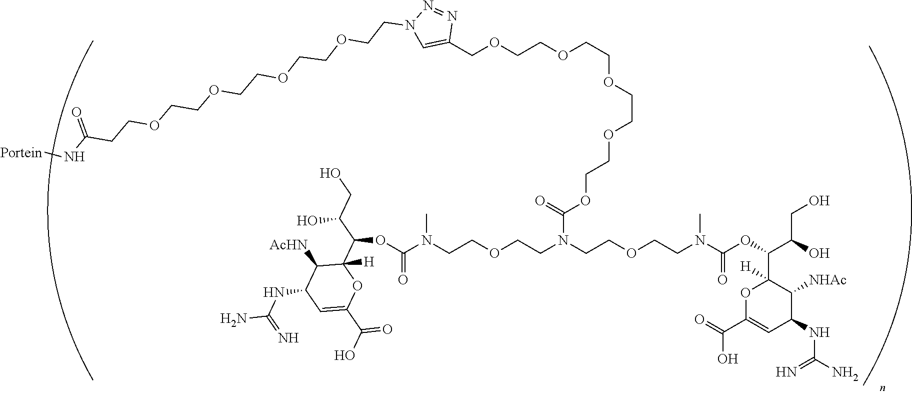 |
| C-2 | 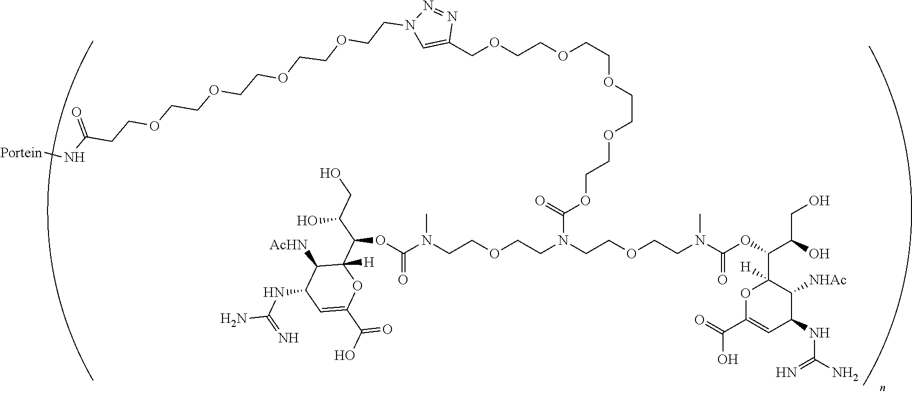 |
| C-3 | 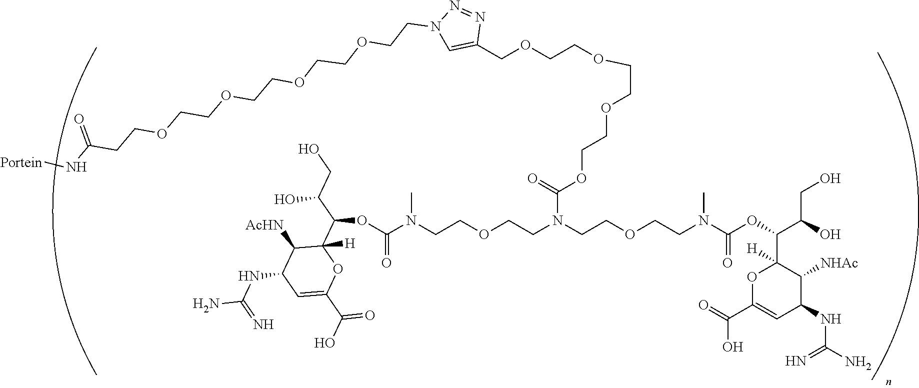 |
| C-4 | 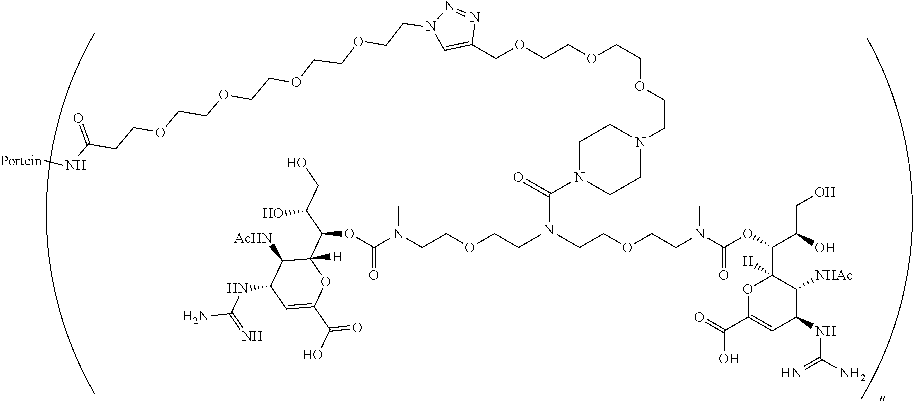 |
| C-5 | 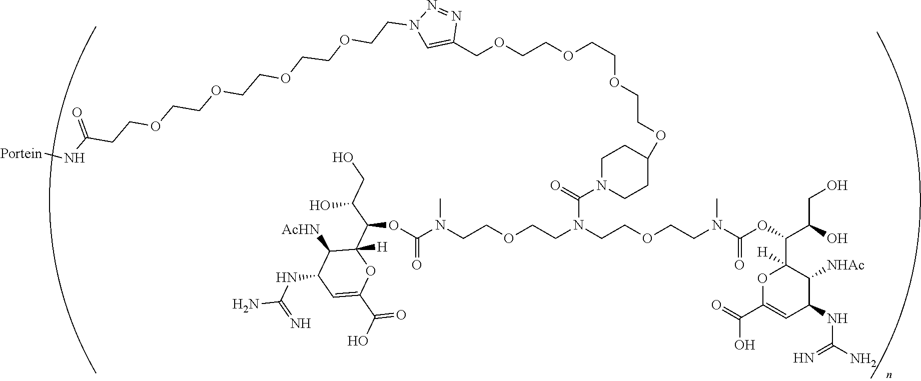 |
| C-6 | 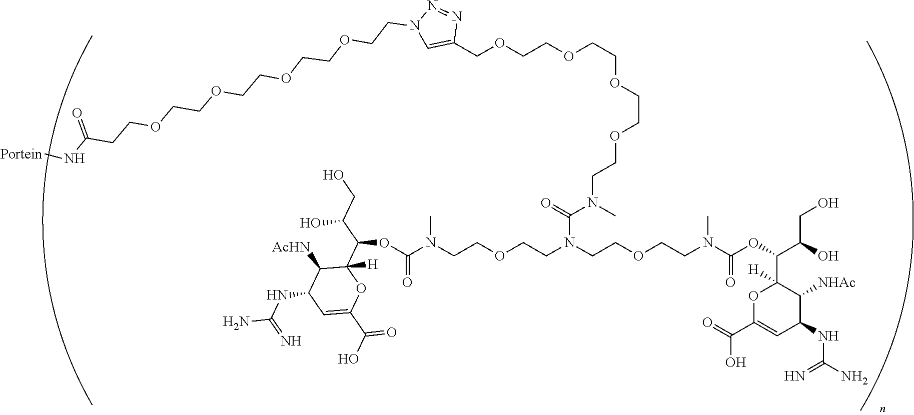 |
| C-7 | 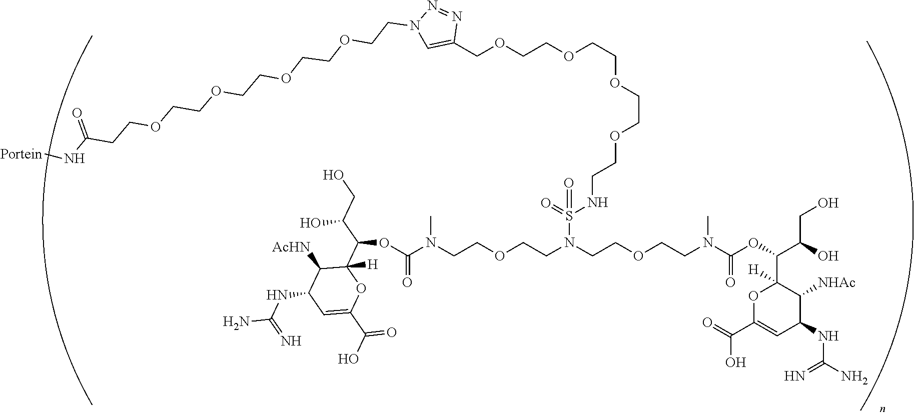 |
| C-8 | 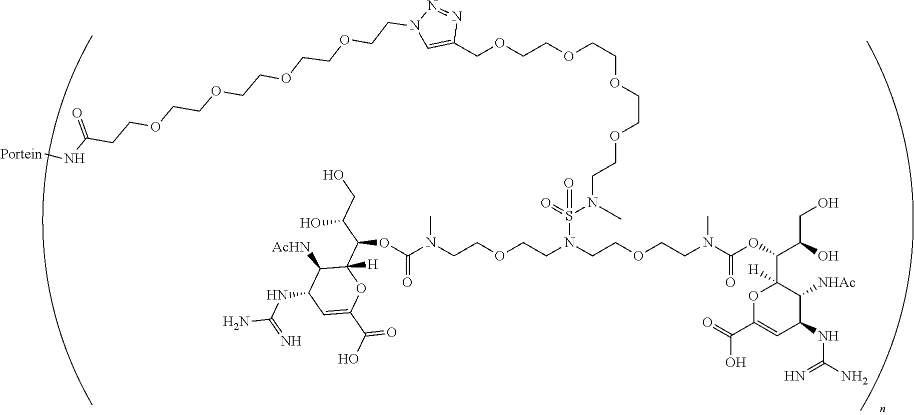 |
| C-9 | 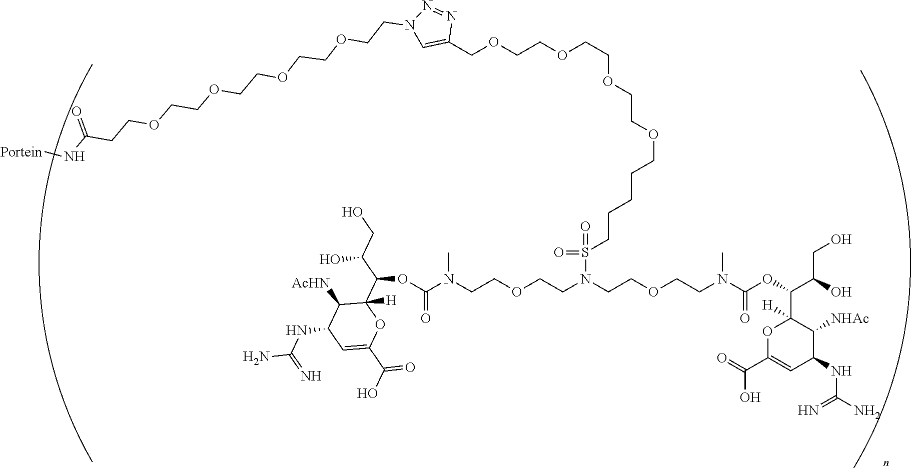 |
| C-10 | 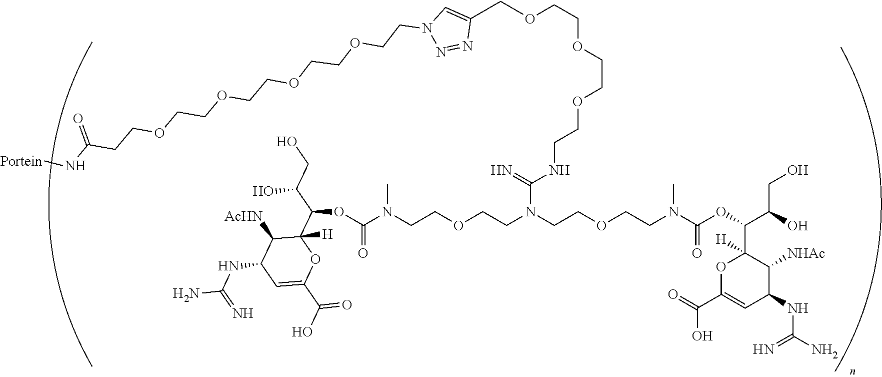 |
| C-11 | 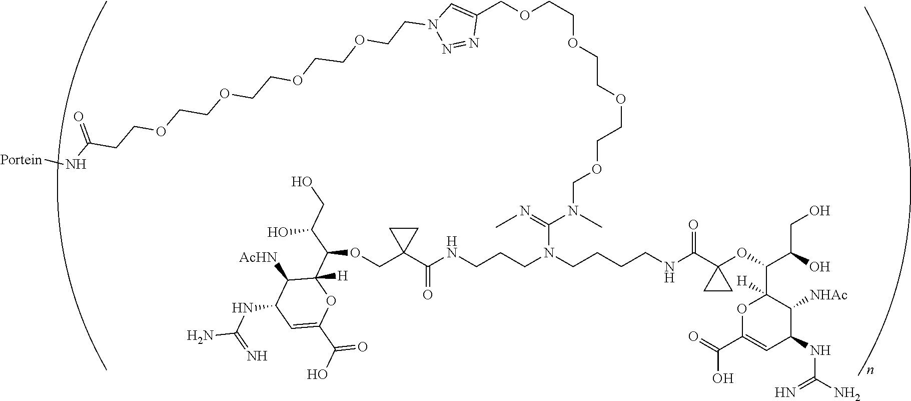 |
| C-12 | 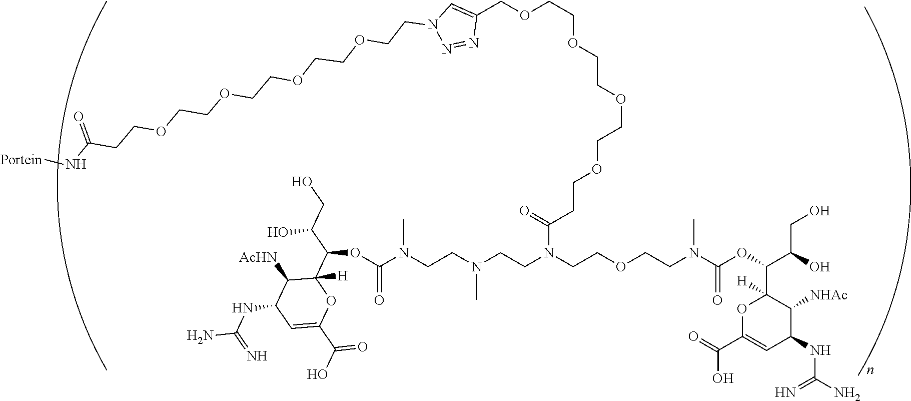 |
| C-13 | 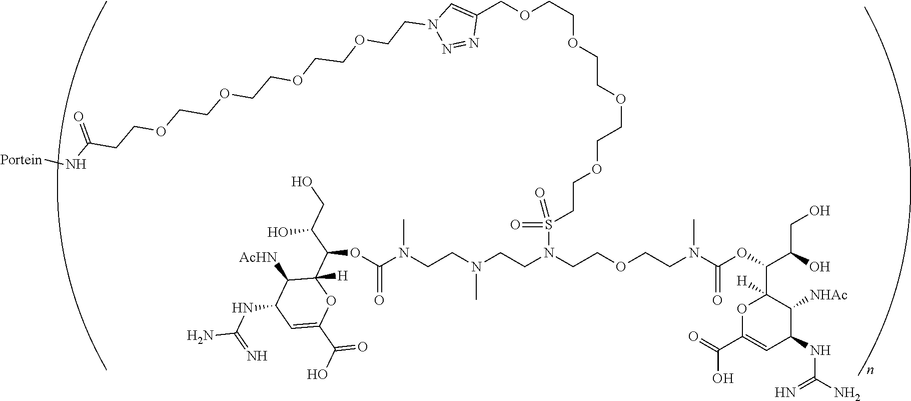 |
| C-14 | 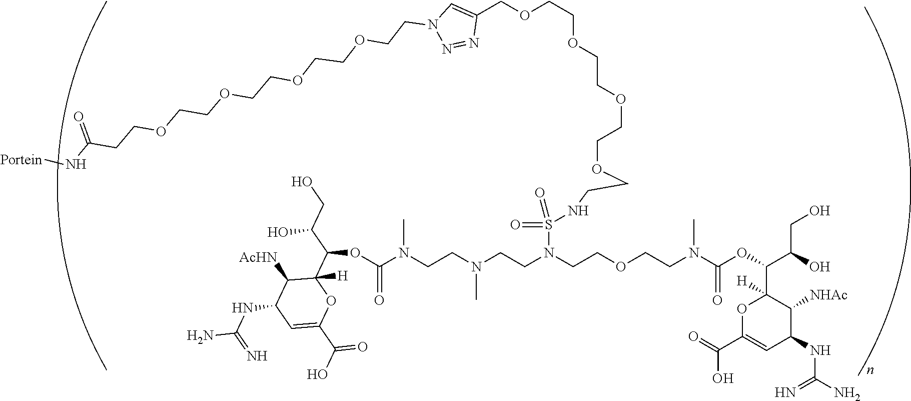 |
| C-15 | 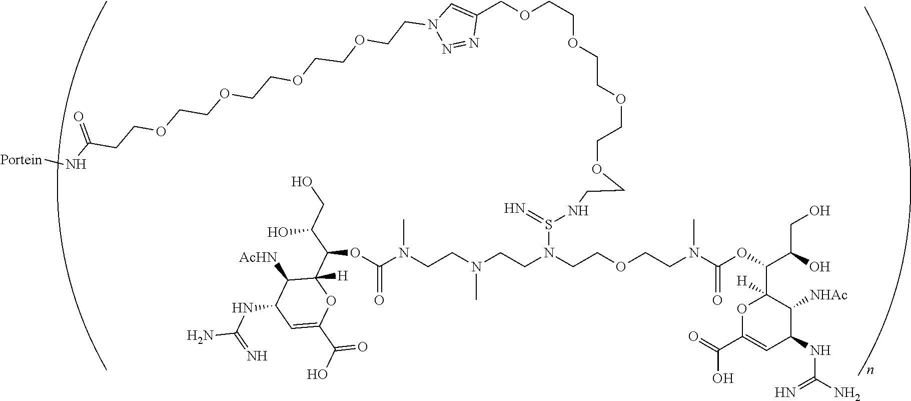 |
| C-16 | 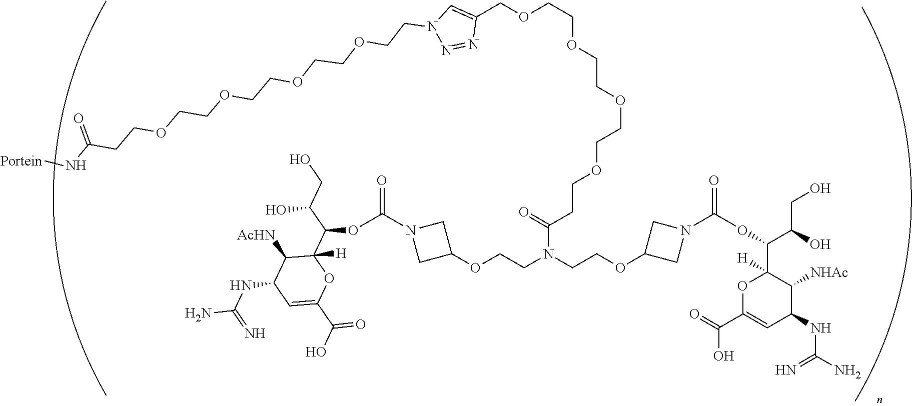 |
| C-17 | 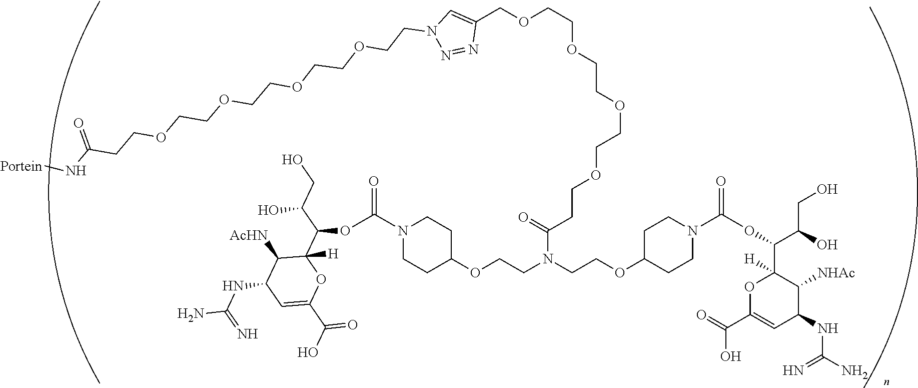 |
| C-18 | 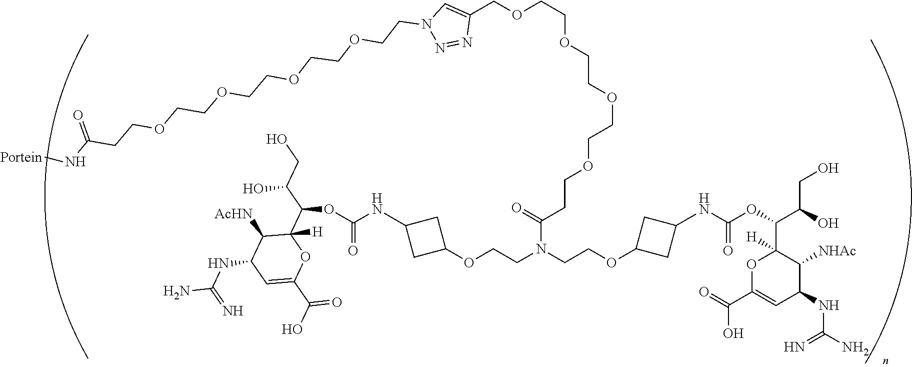 |
| C-19 | 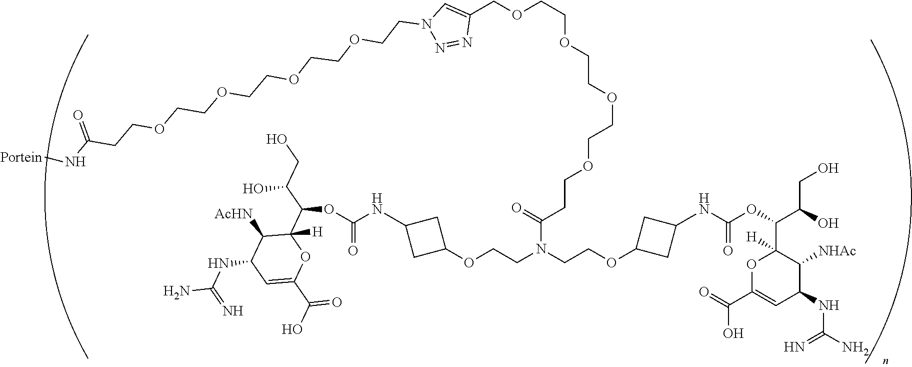 |
| C-20 | 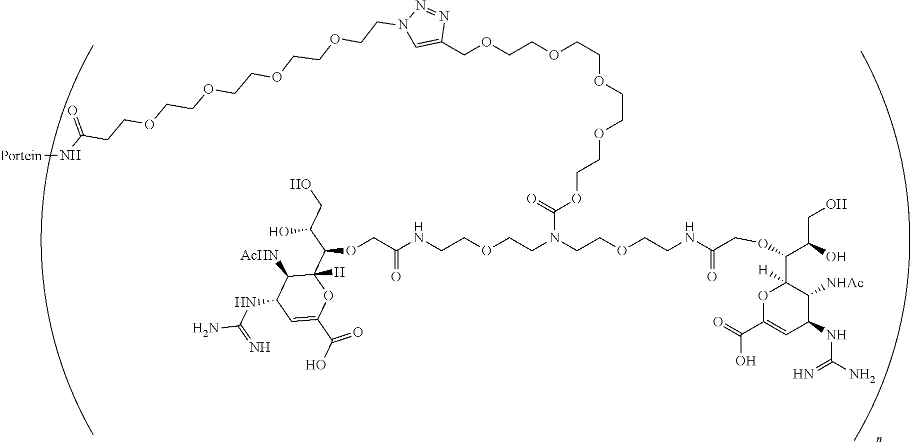 |
| C-21 | 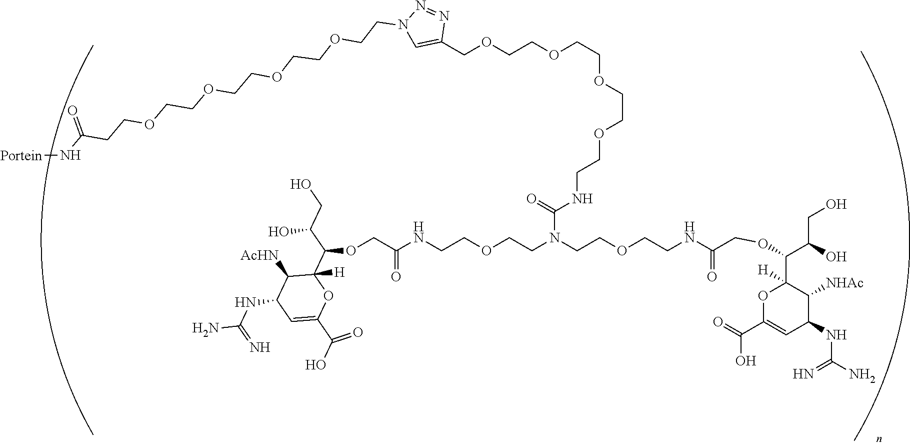 |
| C-22 | 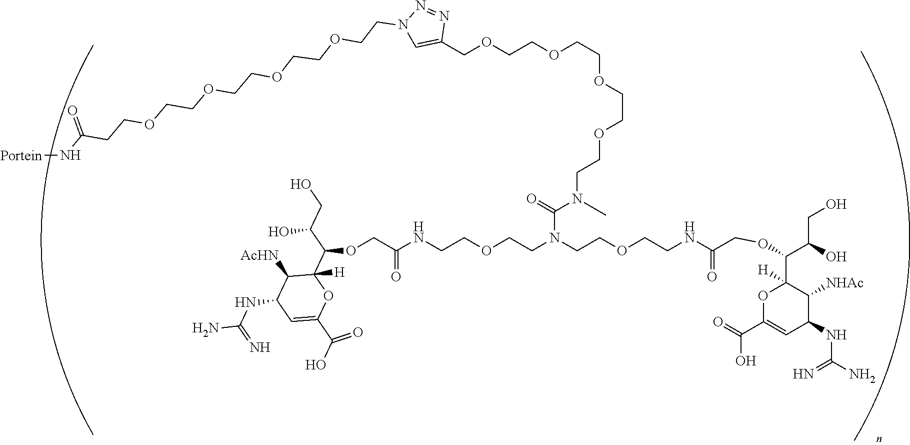 |
| C-23 | 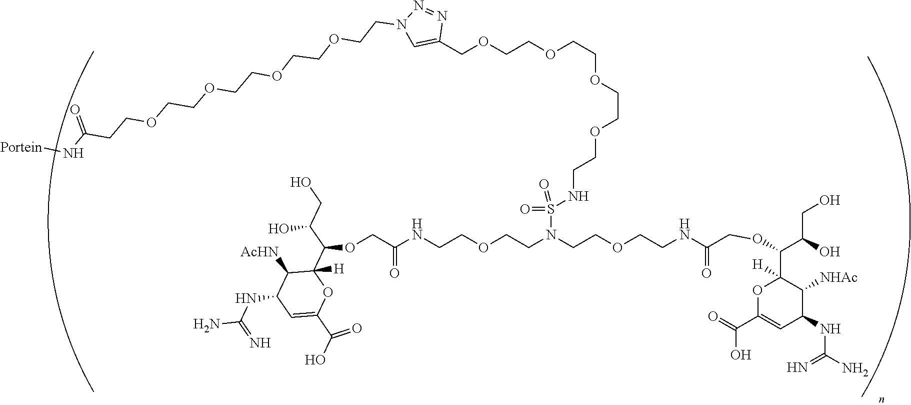 |
| C-24 | 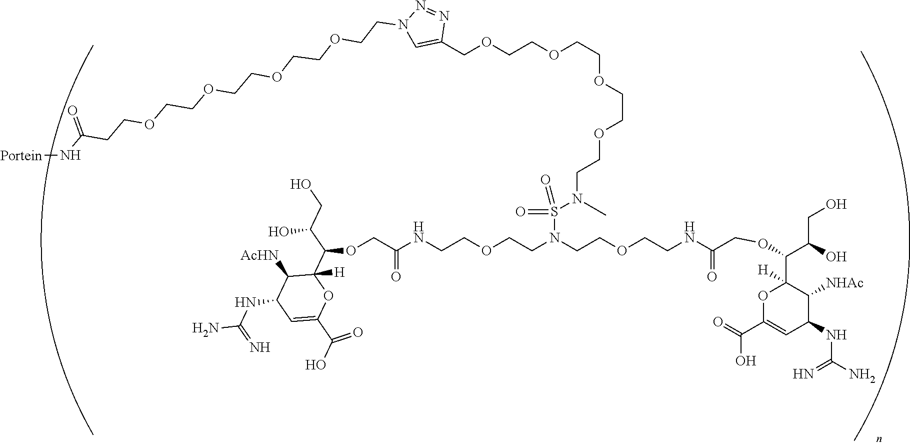 |
| C-25 | 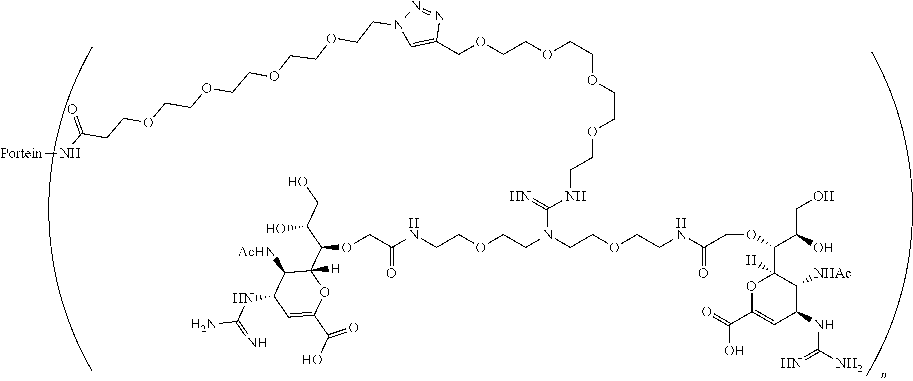 |
| C-26 | 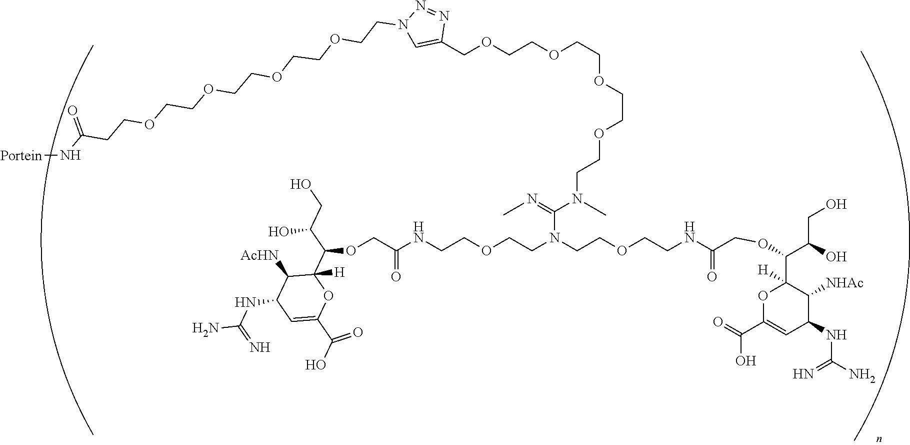 |
| C-27 | 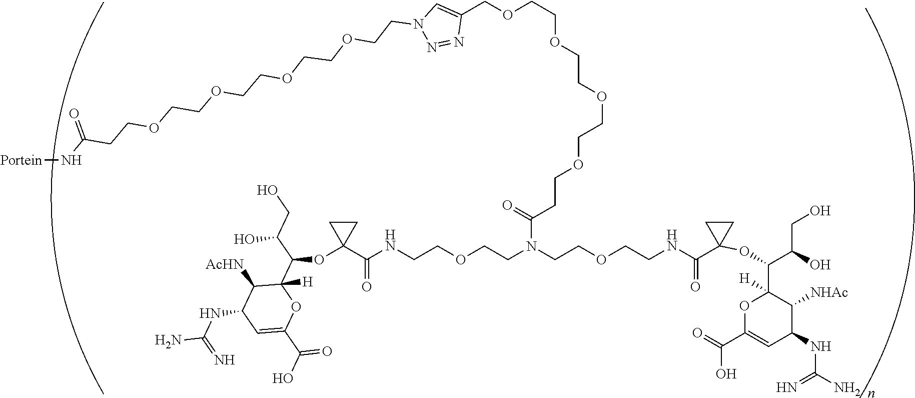 |
| C-28 | 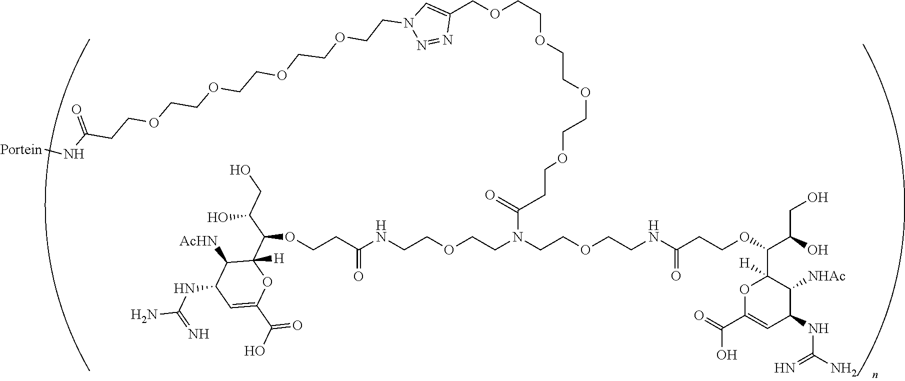 |
| C-29 | 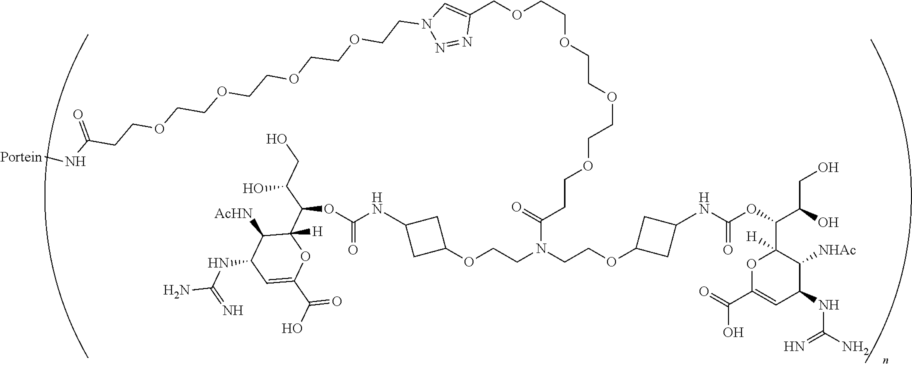 |
| C-30 | 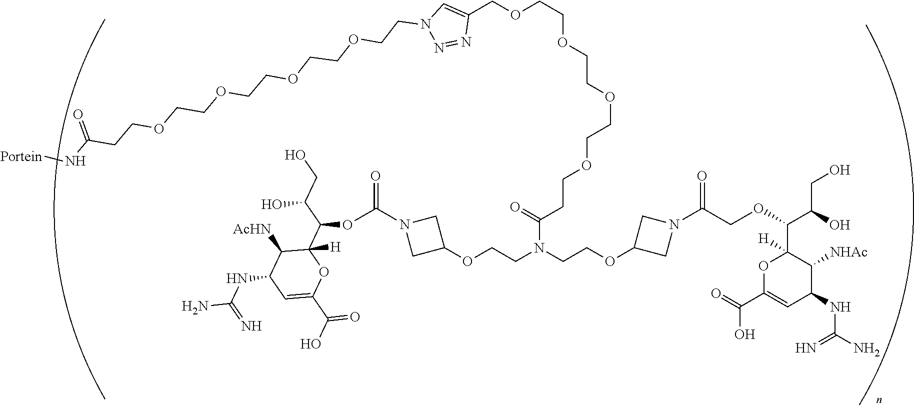 |
| C-31 | 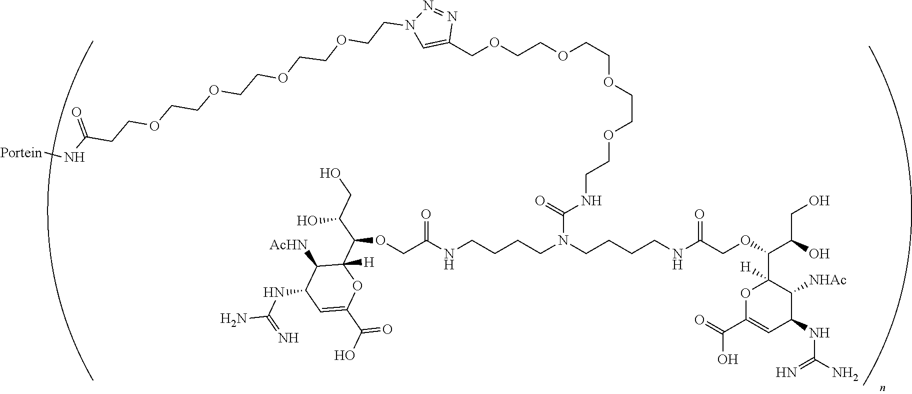 |
| C-32 | 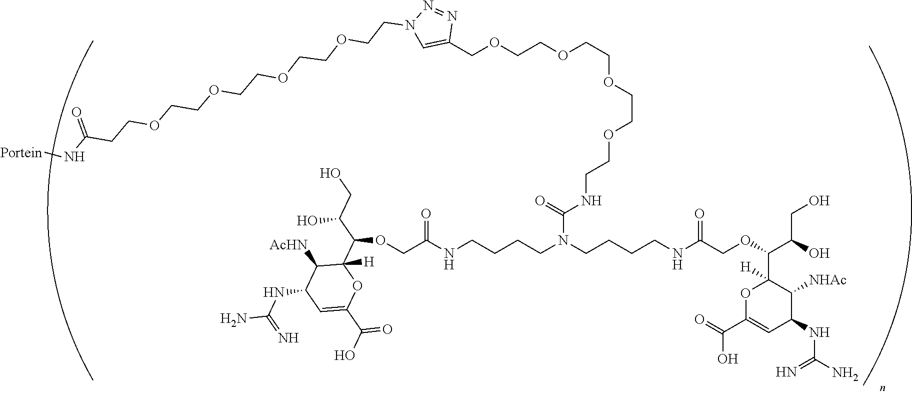 |
| C-33 | 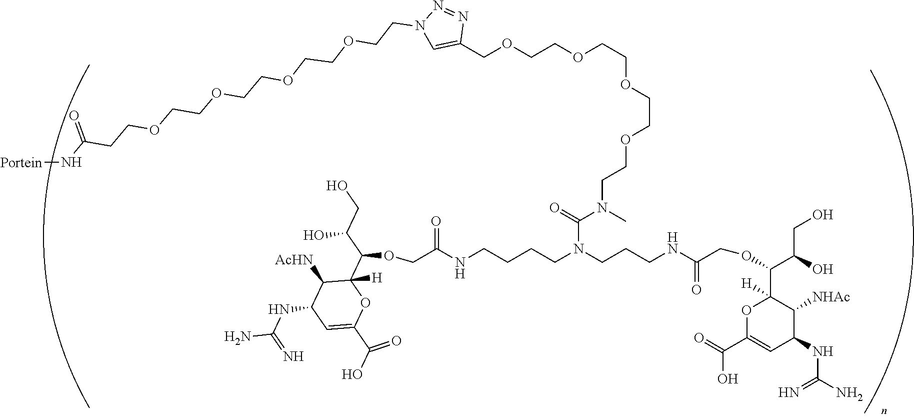 |
| C-34 | 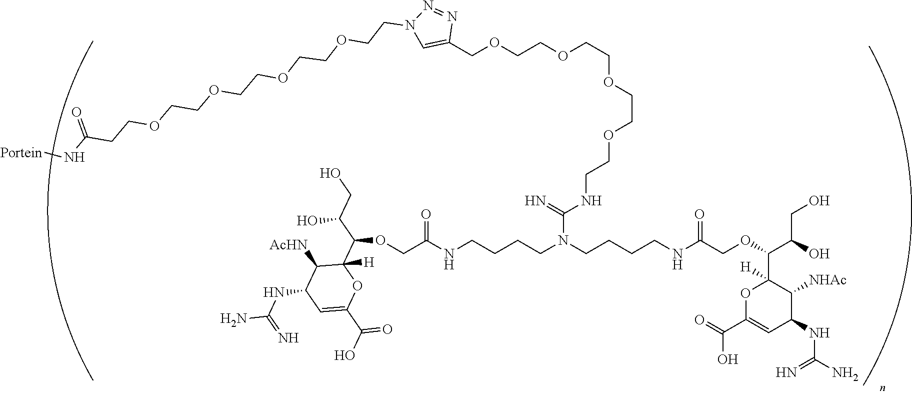 |
| C-35 | 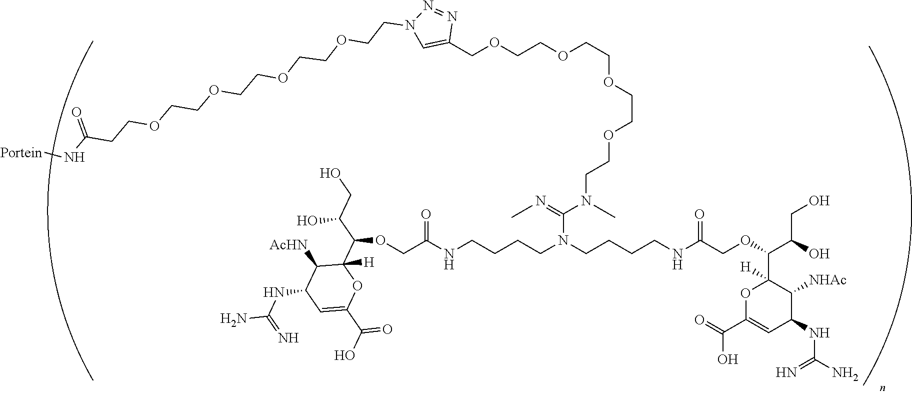 |
| C-36 | 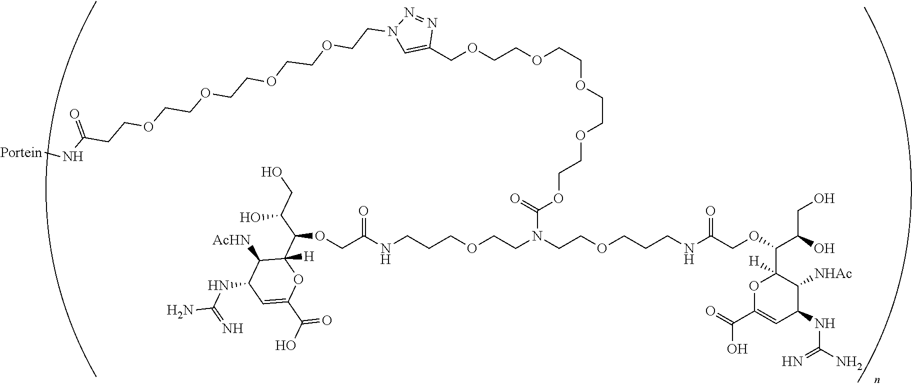 |
| C-37 | 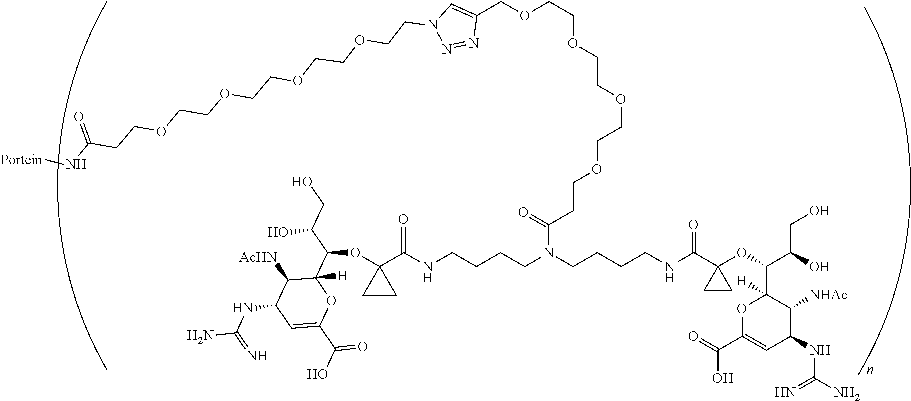 |
| C-38 | 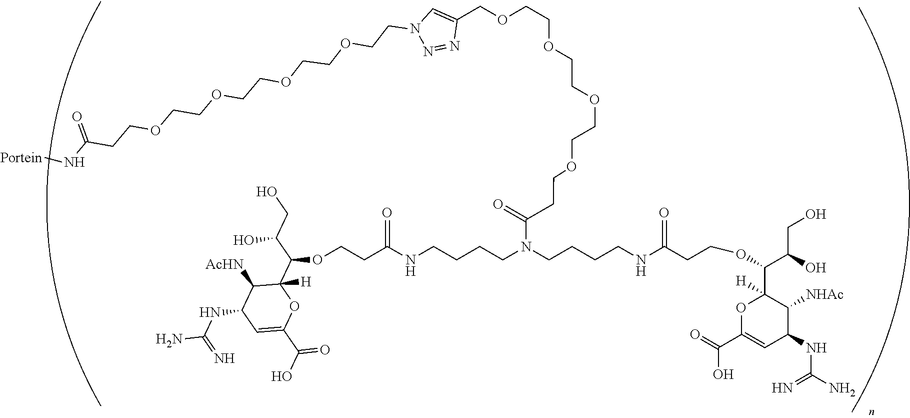 |
| C-39 | 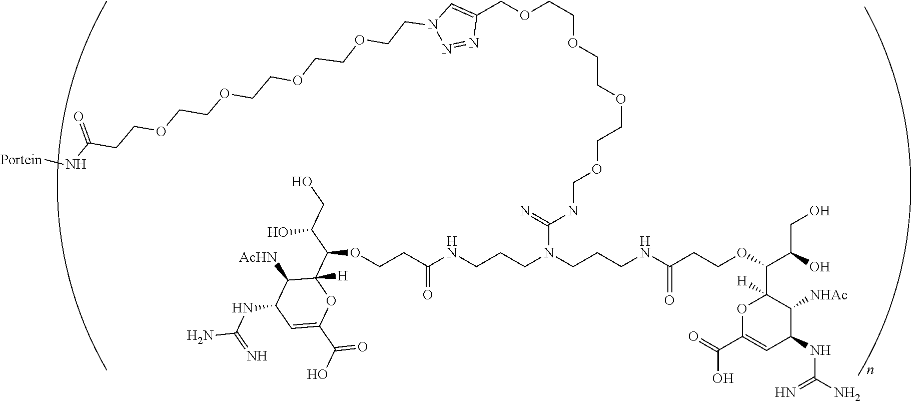 |
| C-40 | 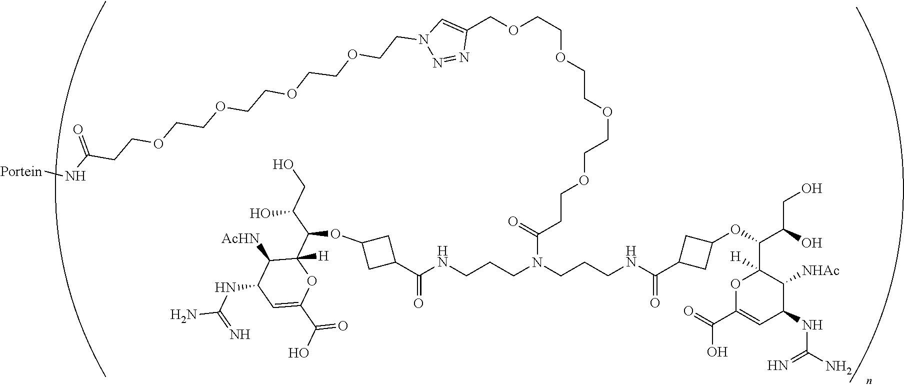 |
| C-41 | 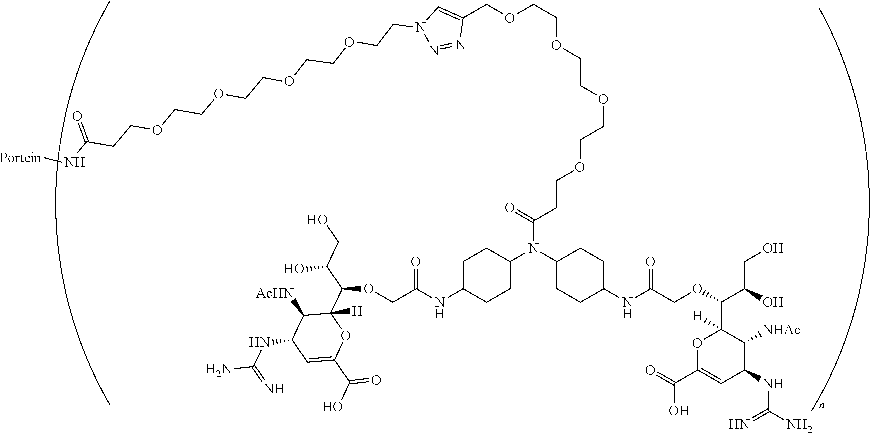 |
| C-42 | 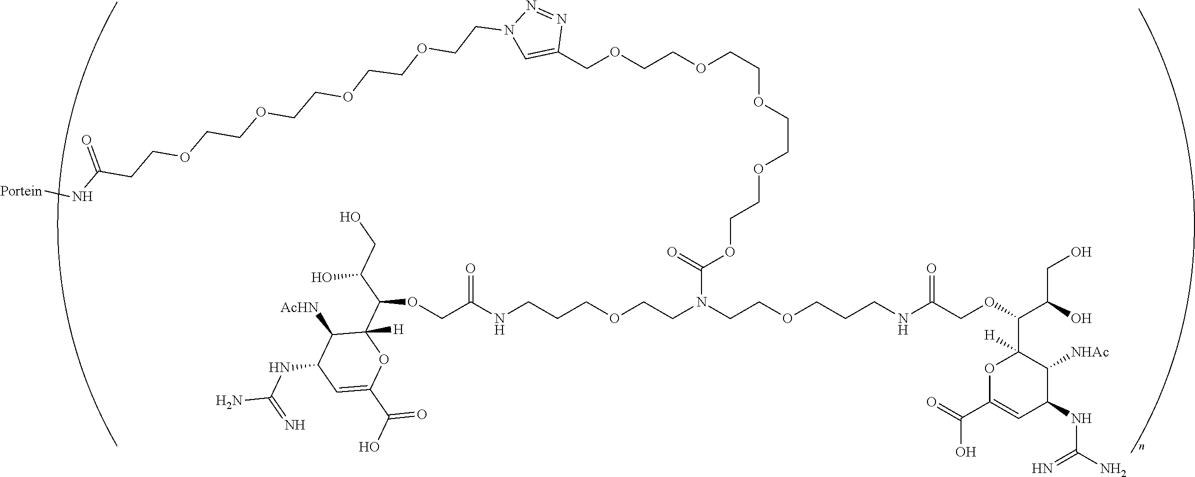 |
| C-43 | 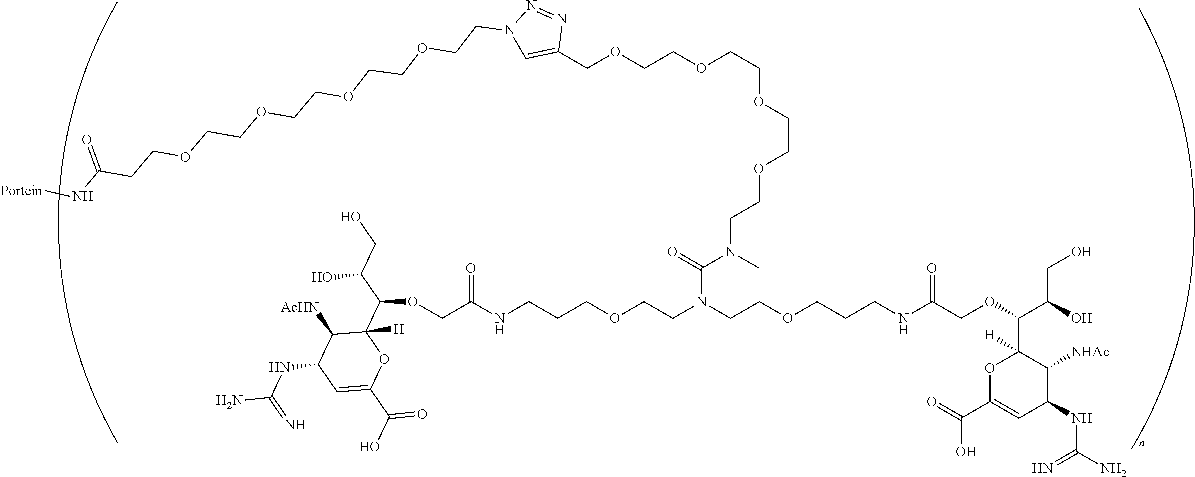 |
| C-44 | 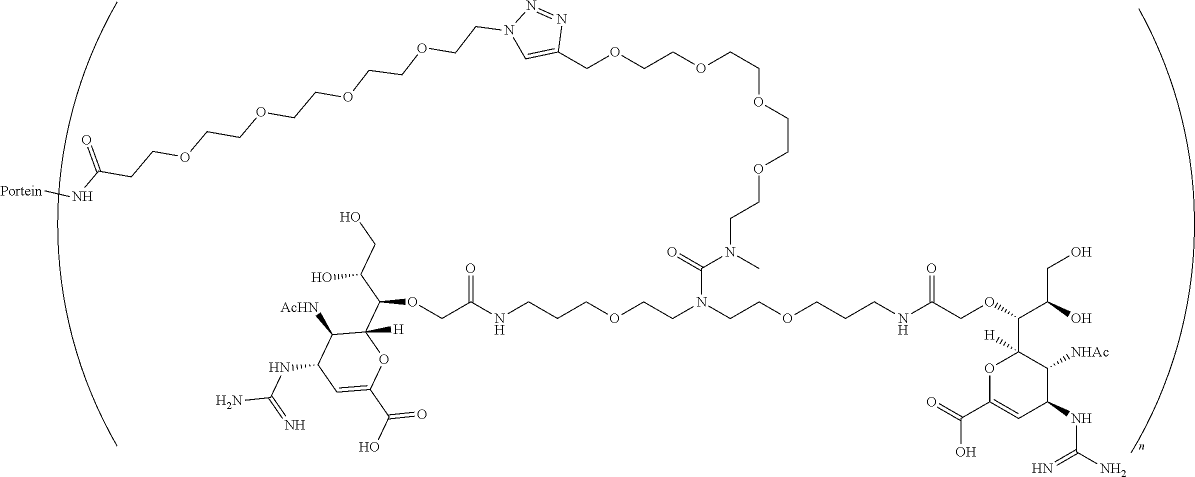 |
| C-45 | 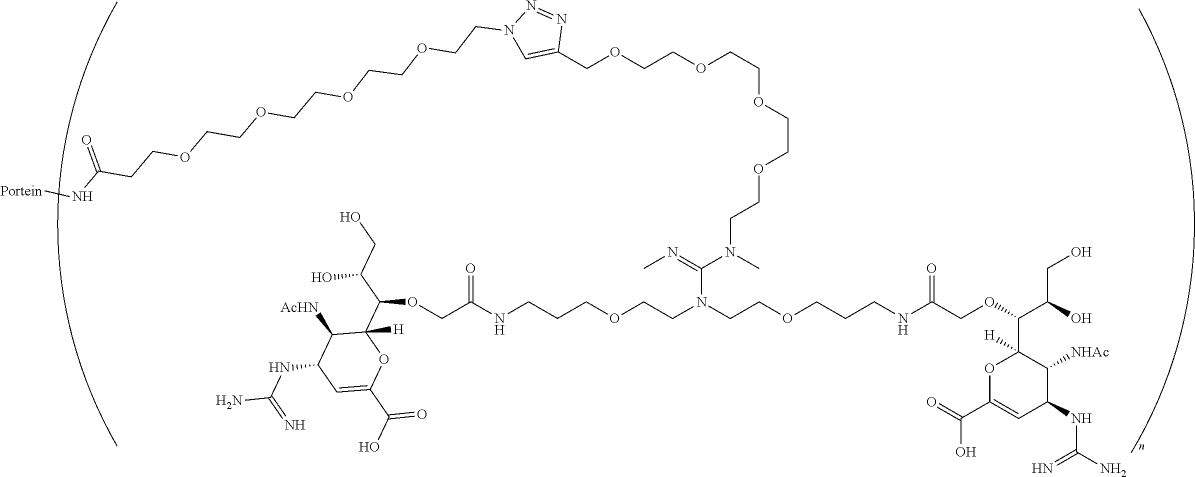 |
| C-46 | 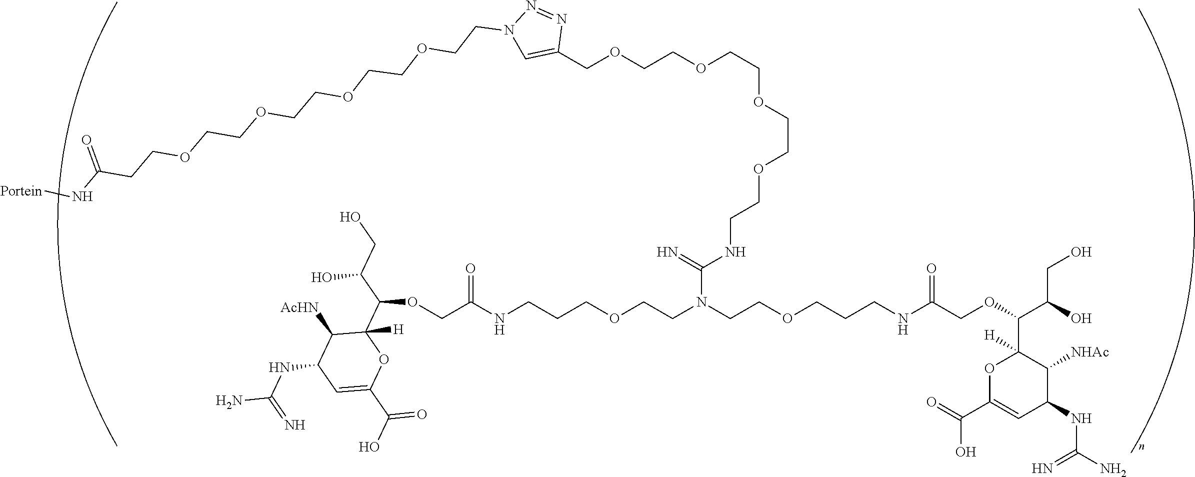 |
| C-47 | 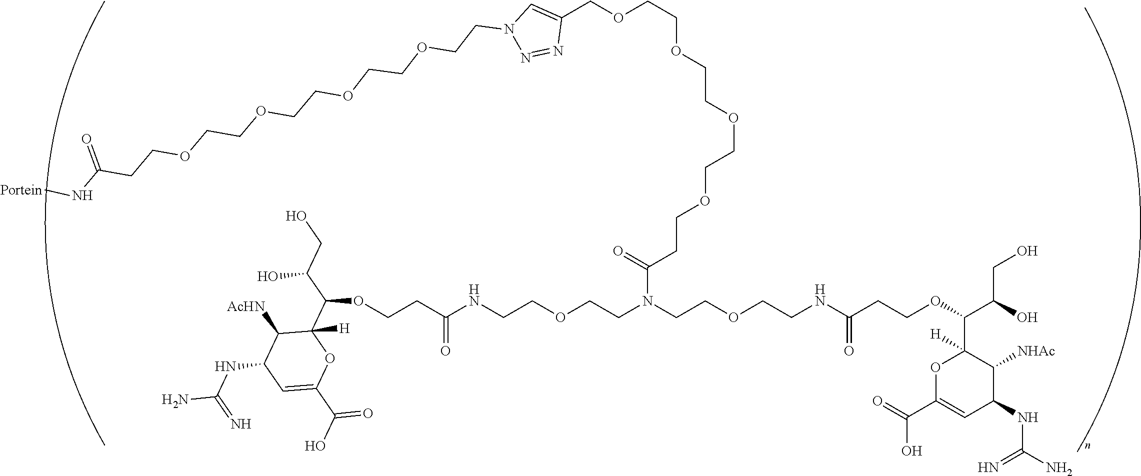 |
| C-48 | 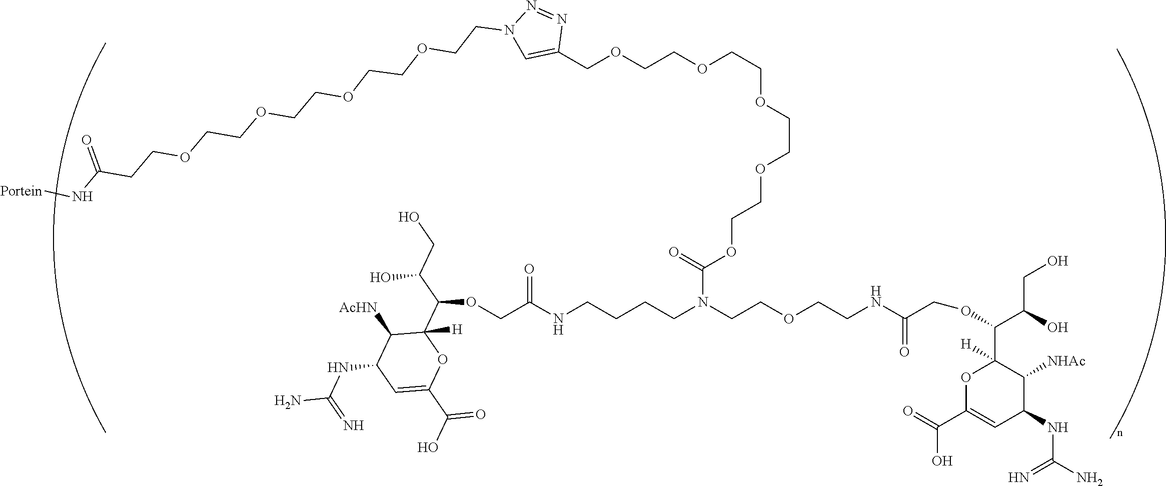 |
| C-49 | 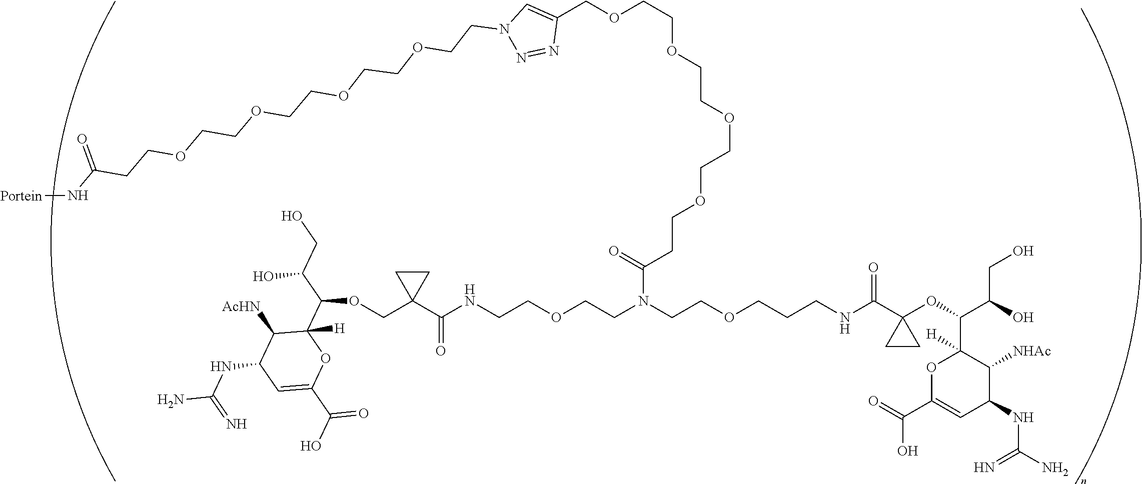 |
| C-50 | 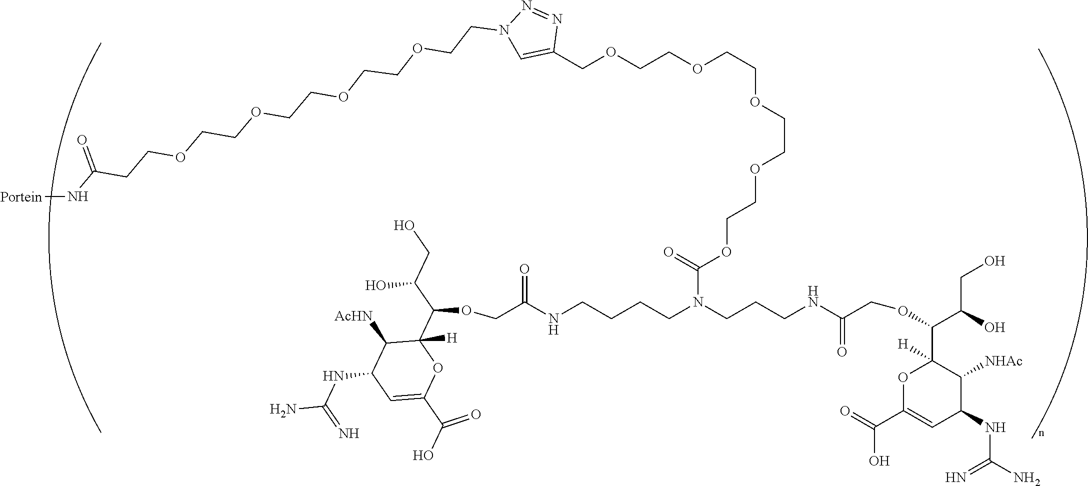 |
| C-51 | 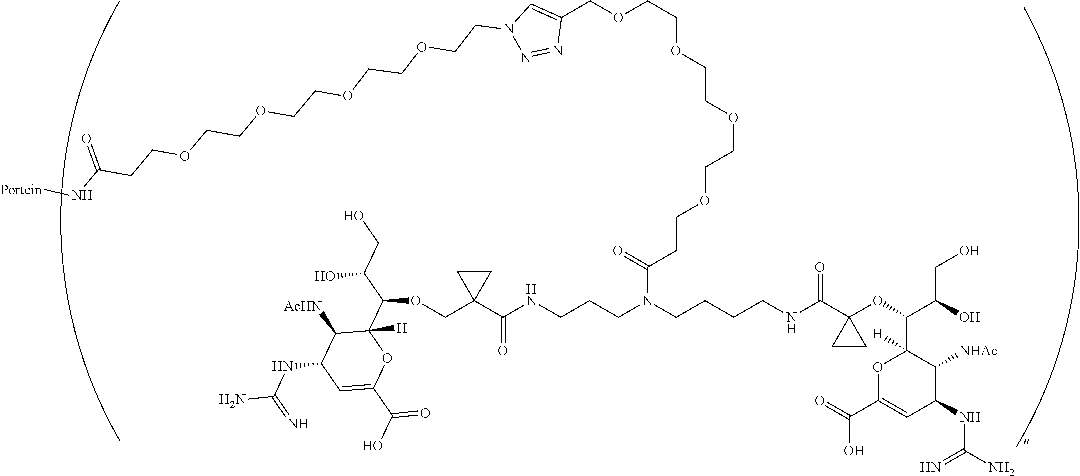 |
| C-52 | 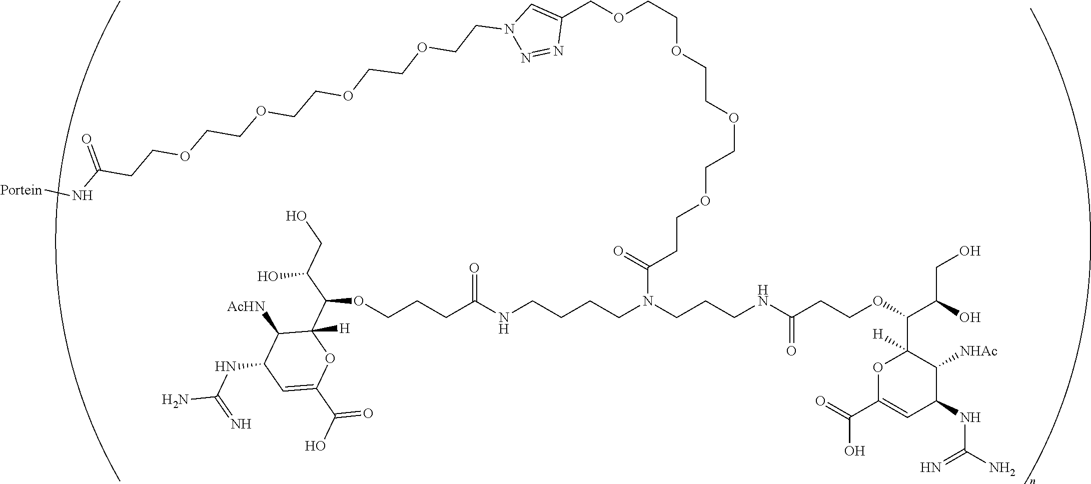 |
| C-53 | 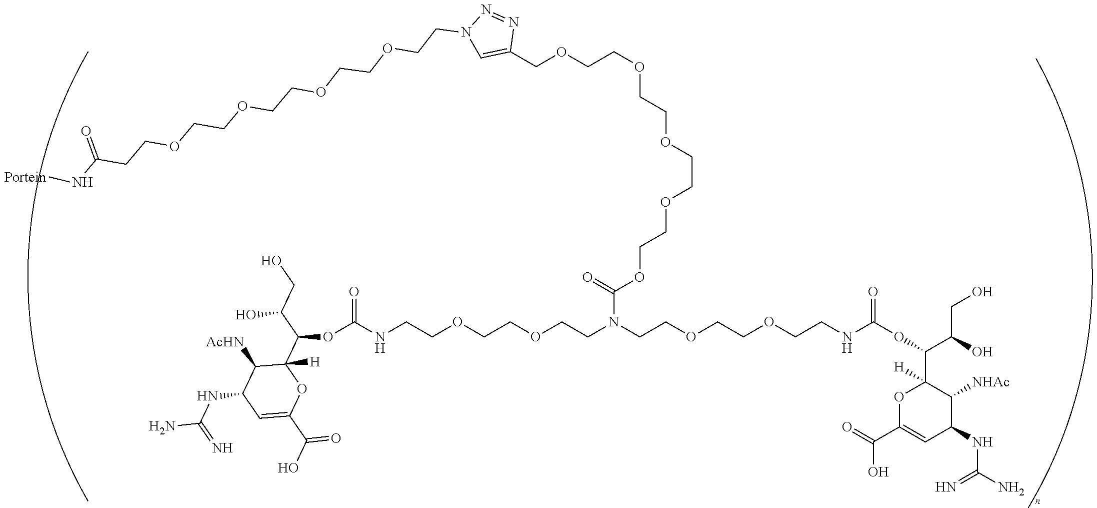 |
| C-54 | 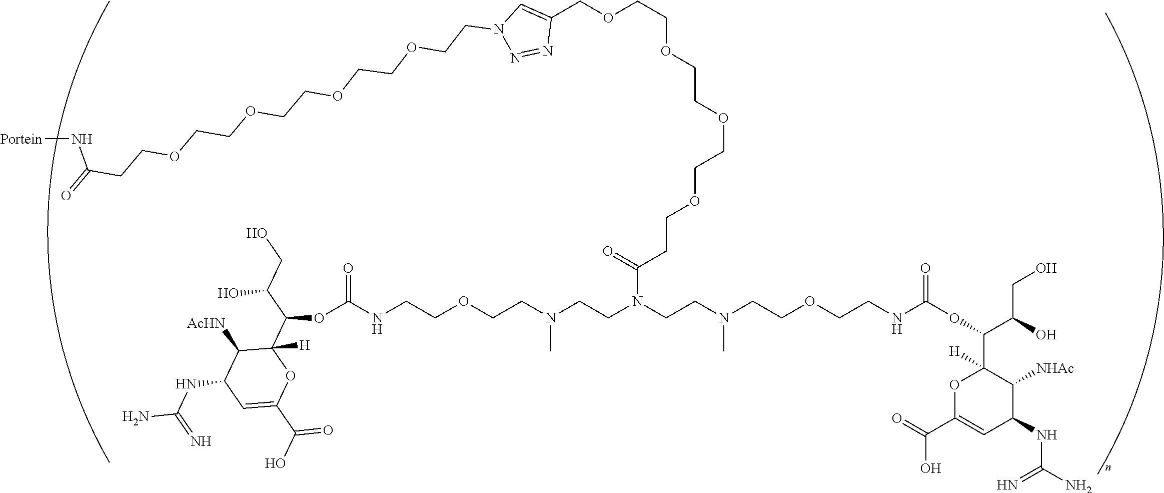 |
| C-55 | 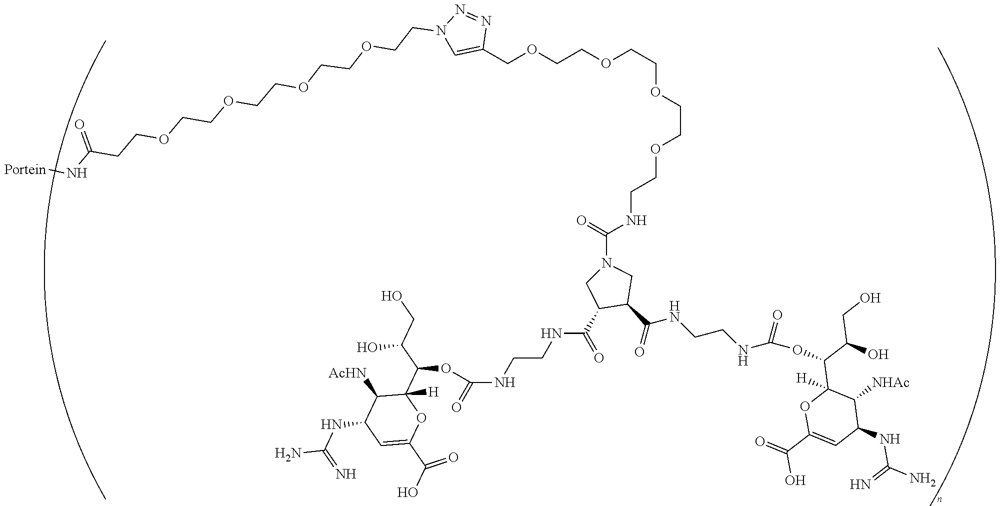 |
| C-56 | 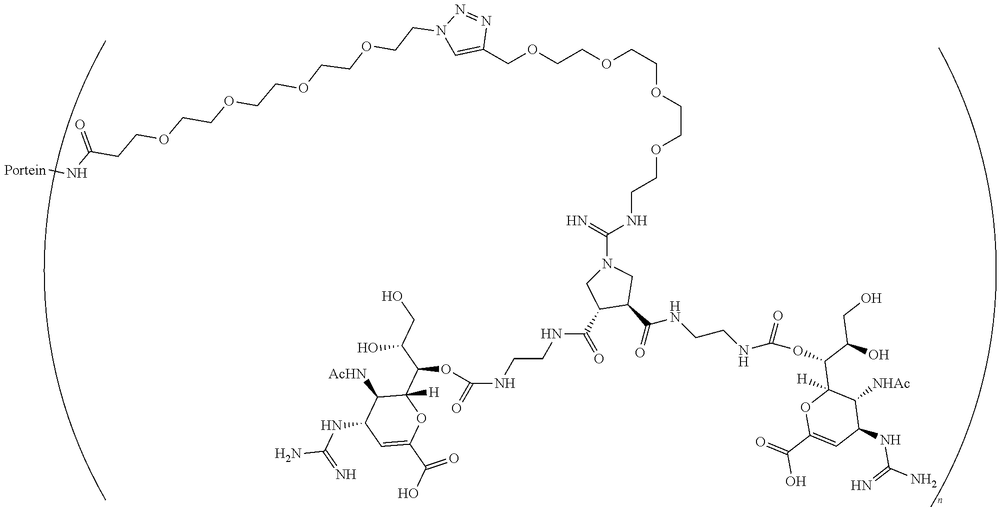 |
| C-57 | 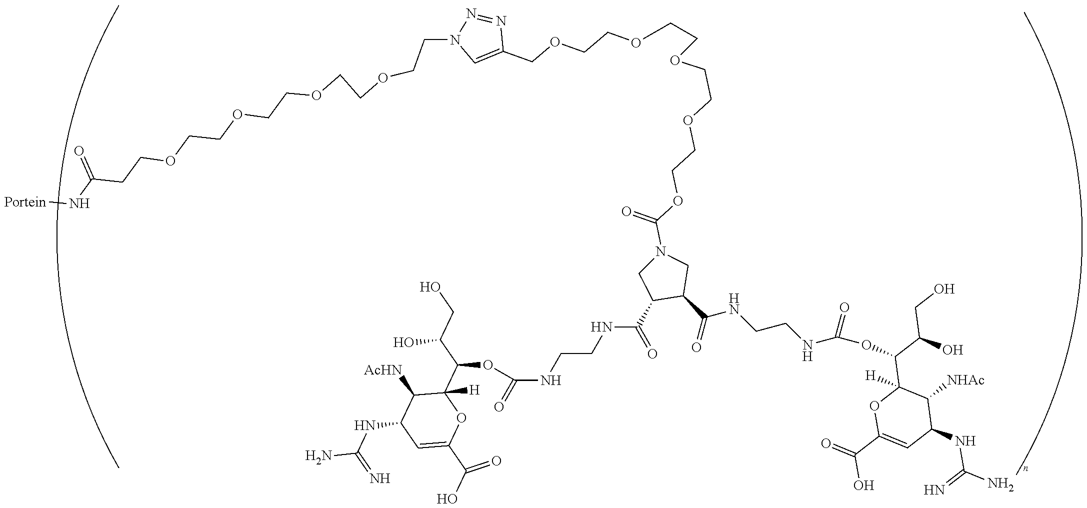 |
| C-58 | 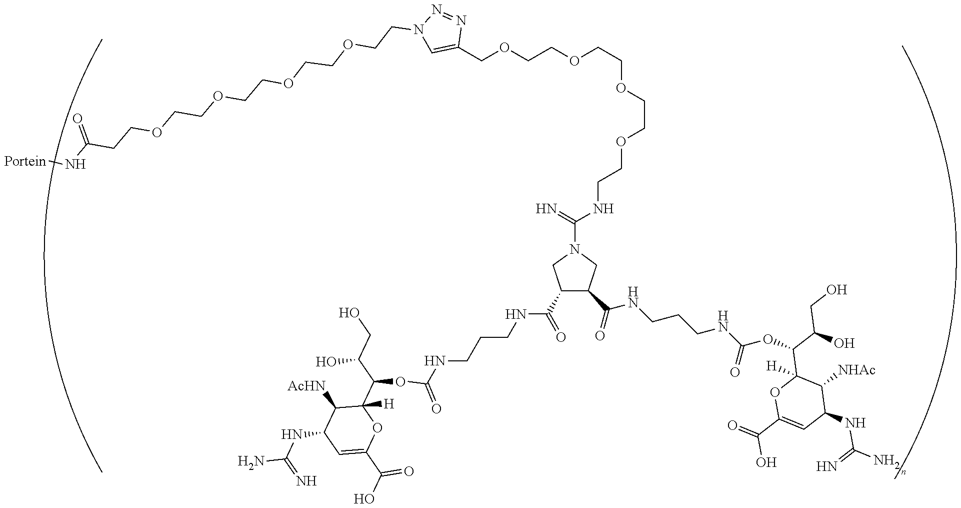 |
| C-59 | 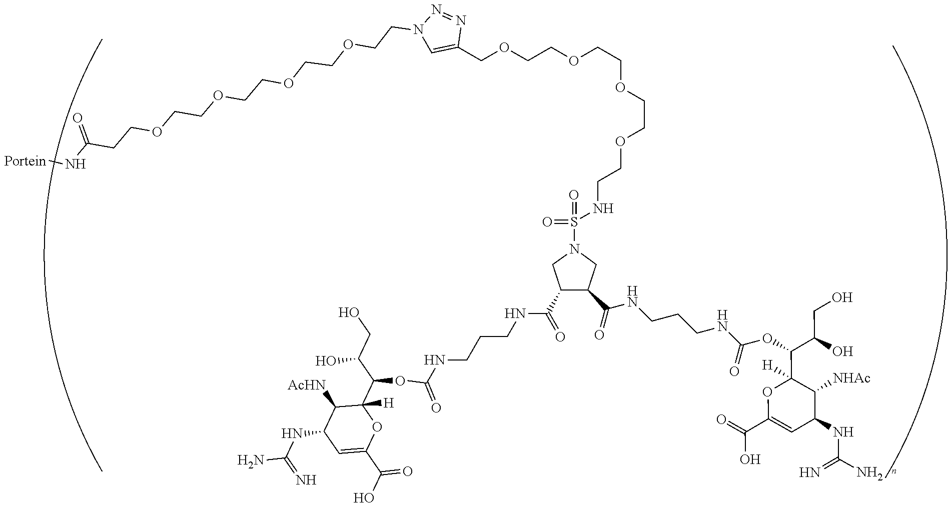 |
| C-60 | 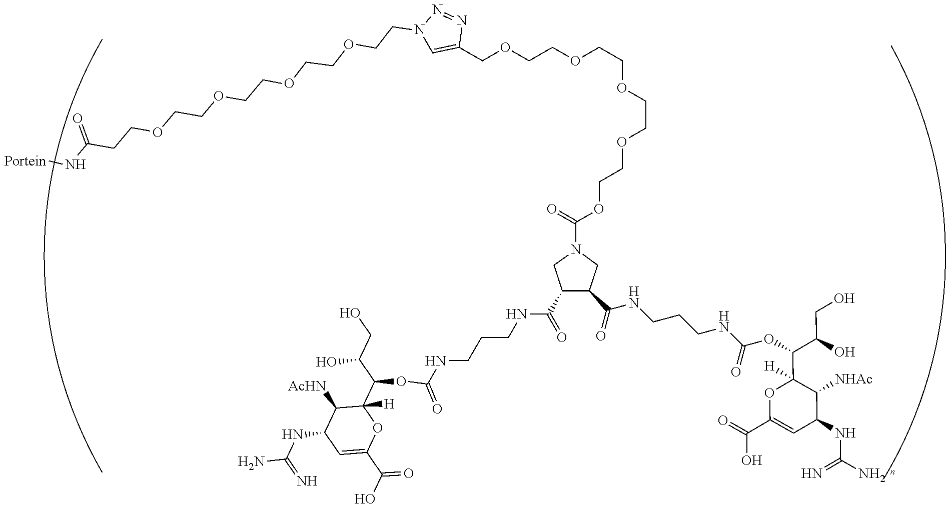 |
| C-61 | 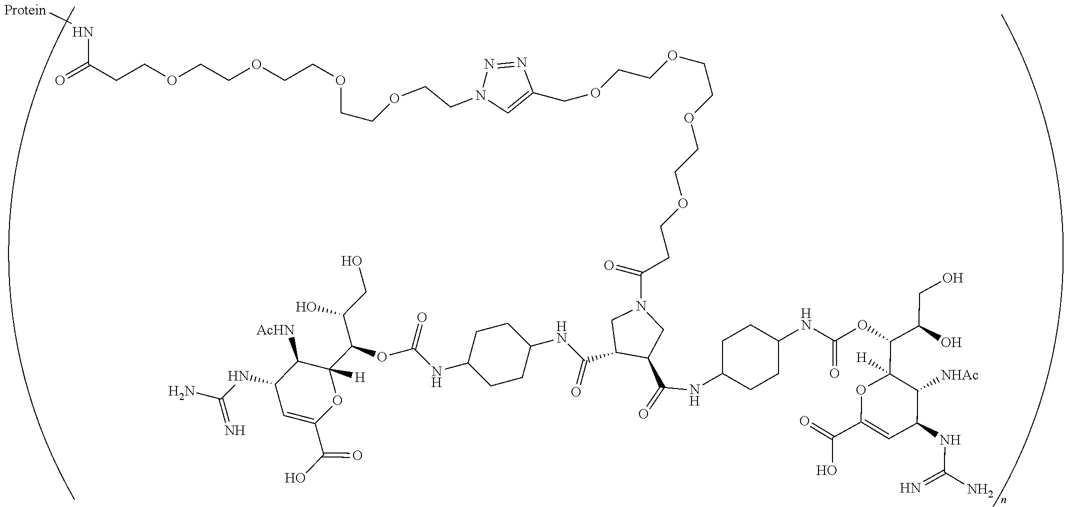 |
| C-62 | 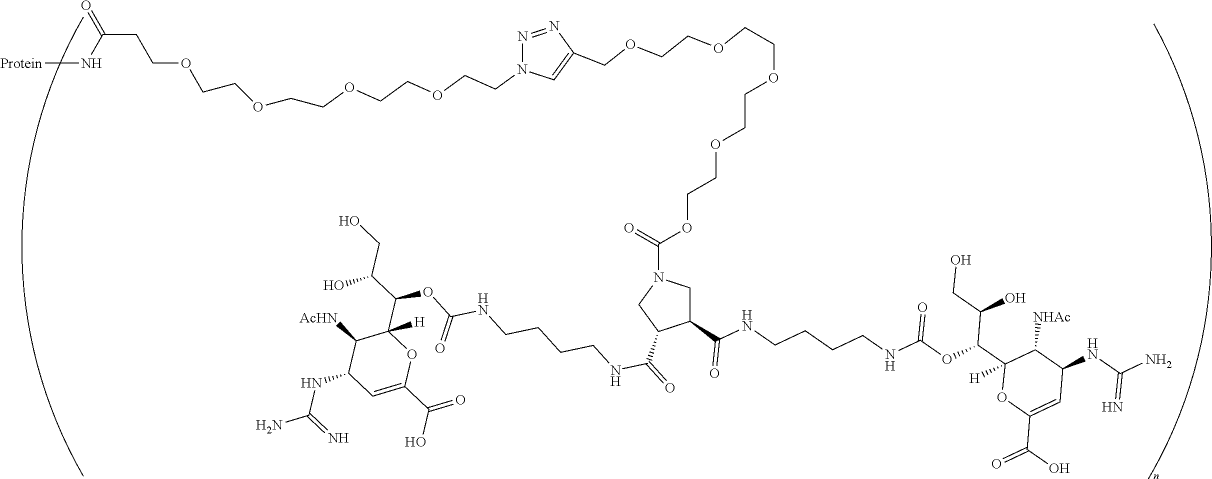 |
| C-63 | 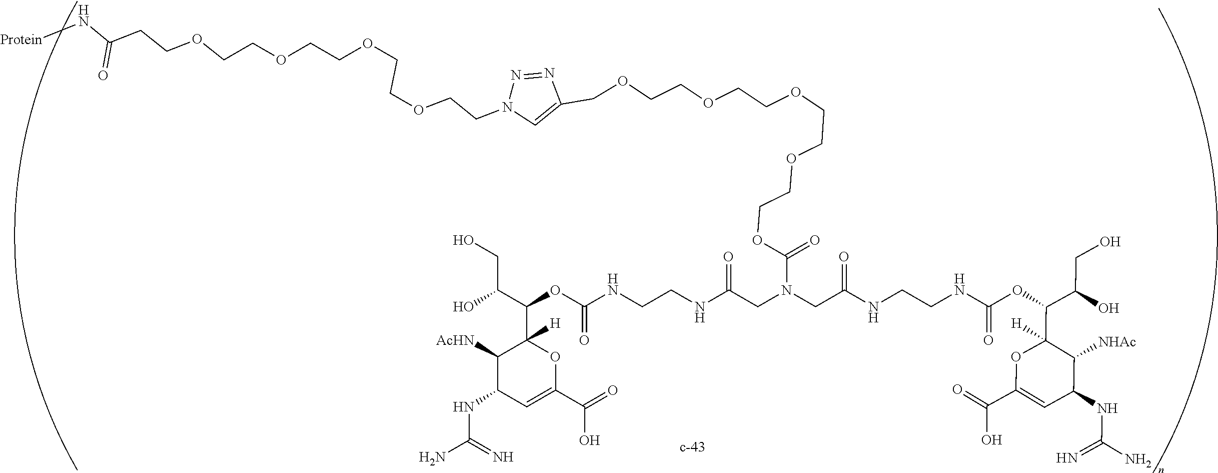 |
| C-64 | 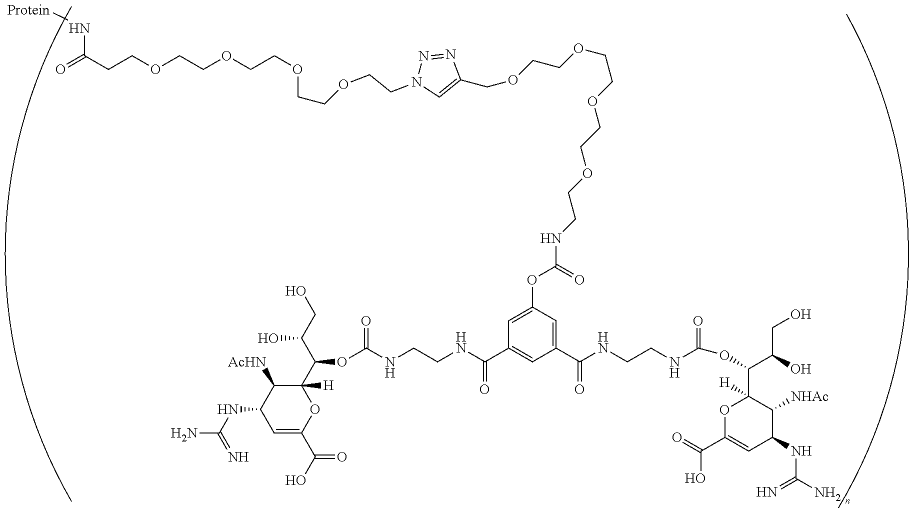 |
| C-65 | 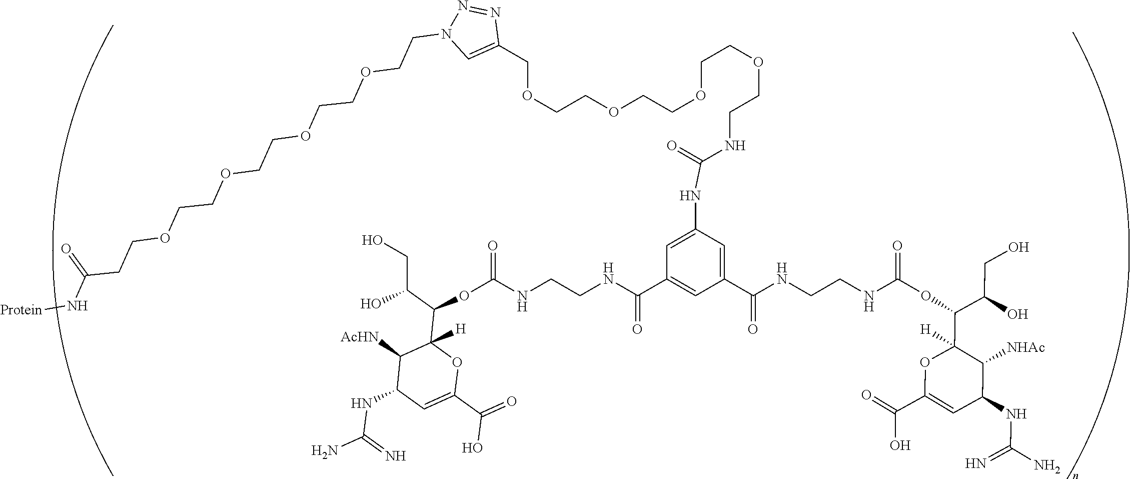 |
| C-66 | 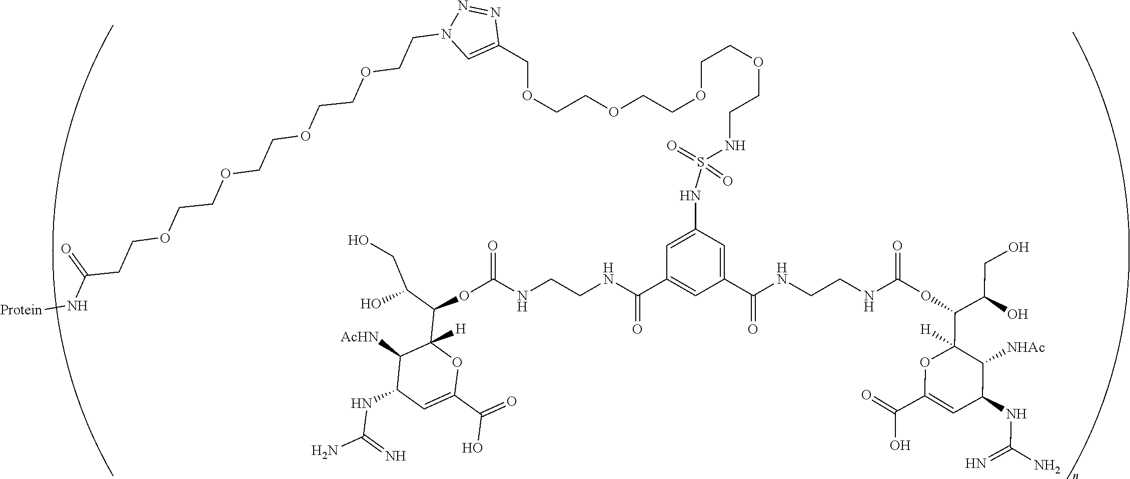 |
| C-67 | 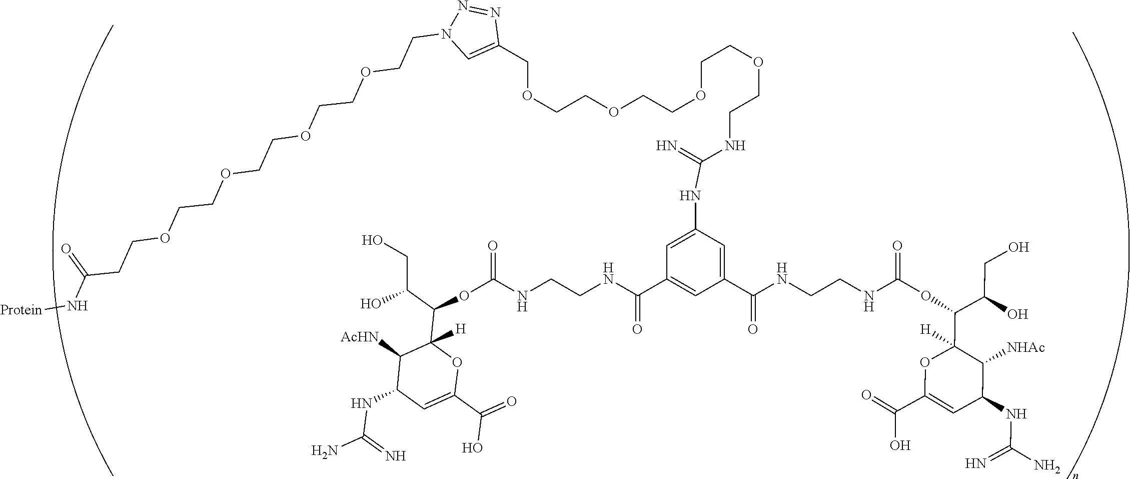 |
| C-68 | 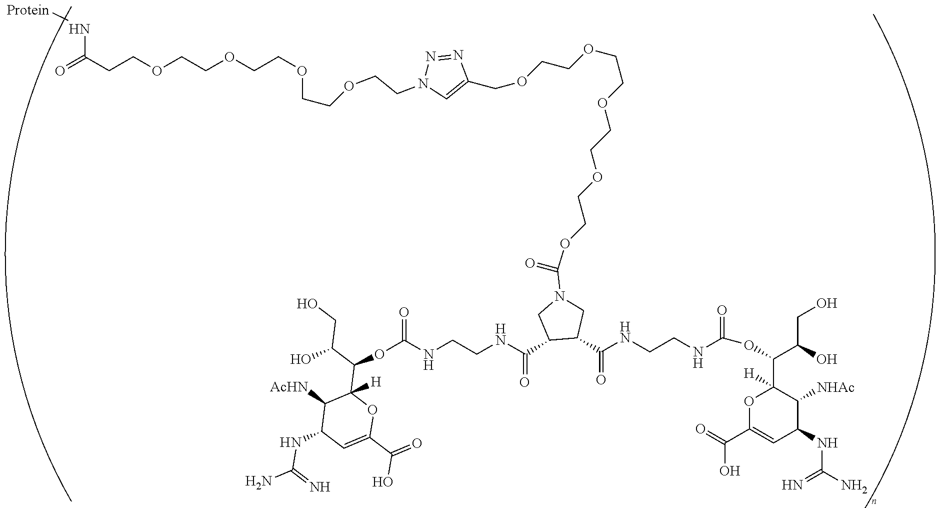 |
| C-69 | 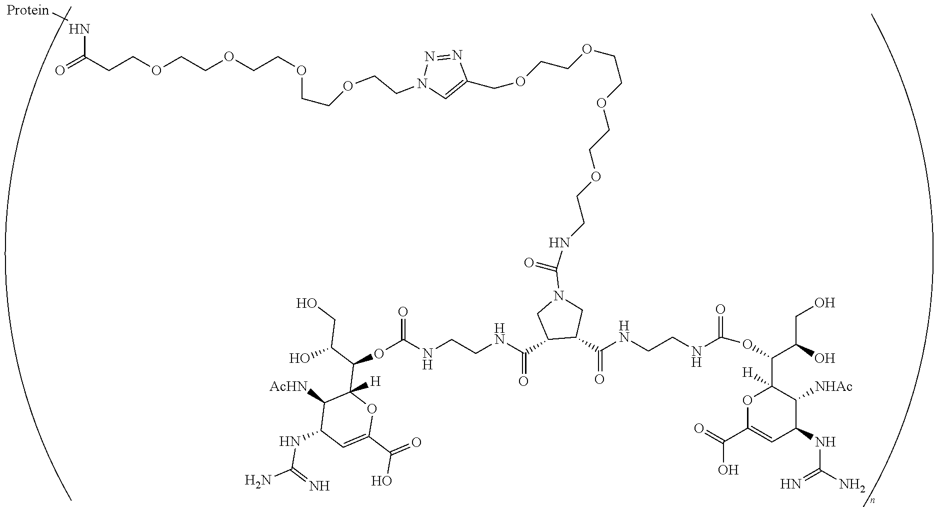 |
| C-70 | 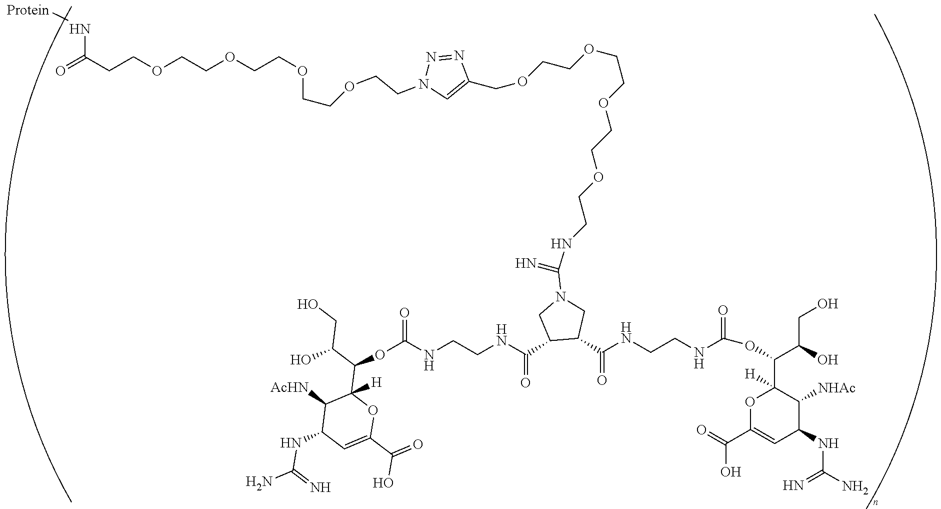 |
| C-71 | 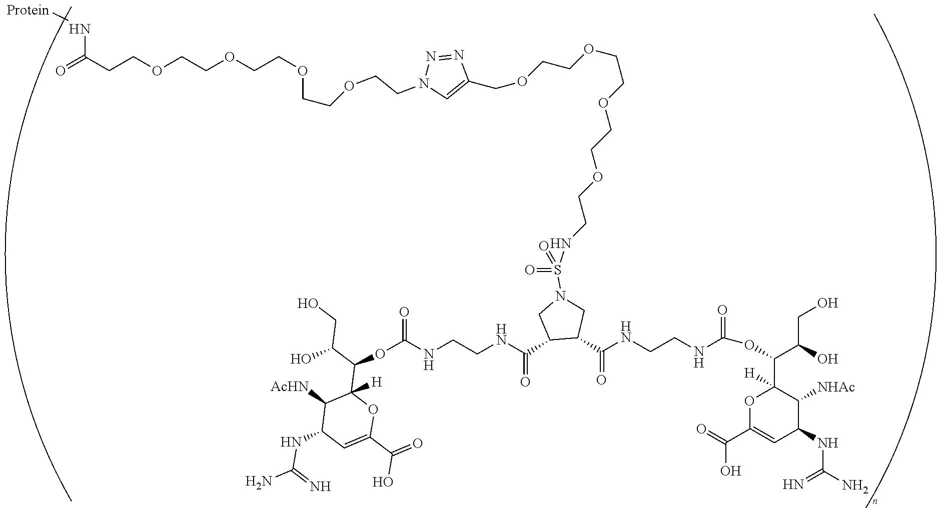 |
| C-72 | 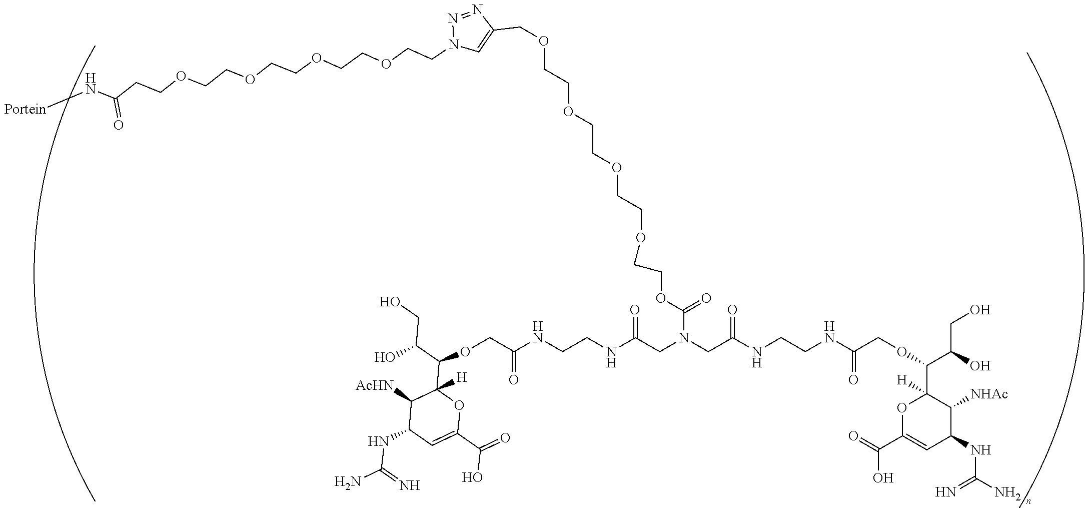 |
| C-73 | 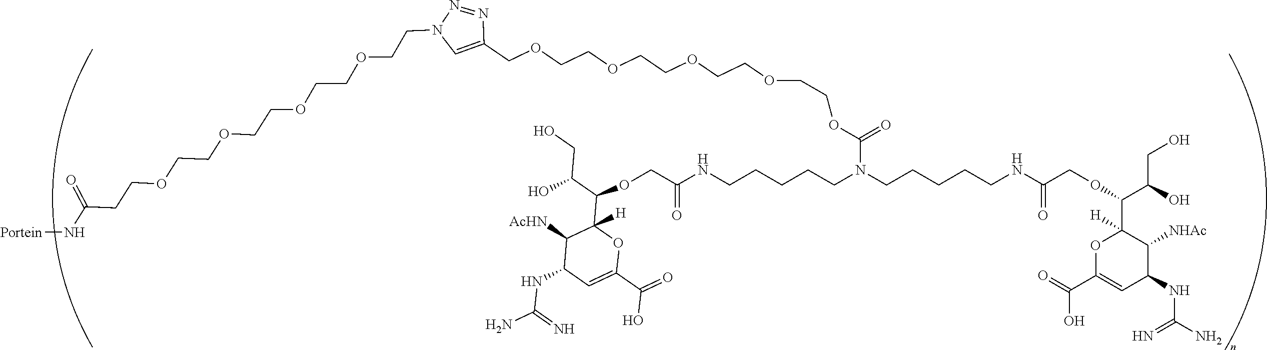 |
| C-74 | 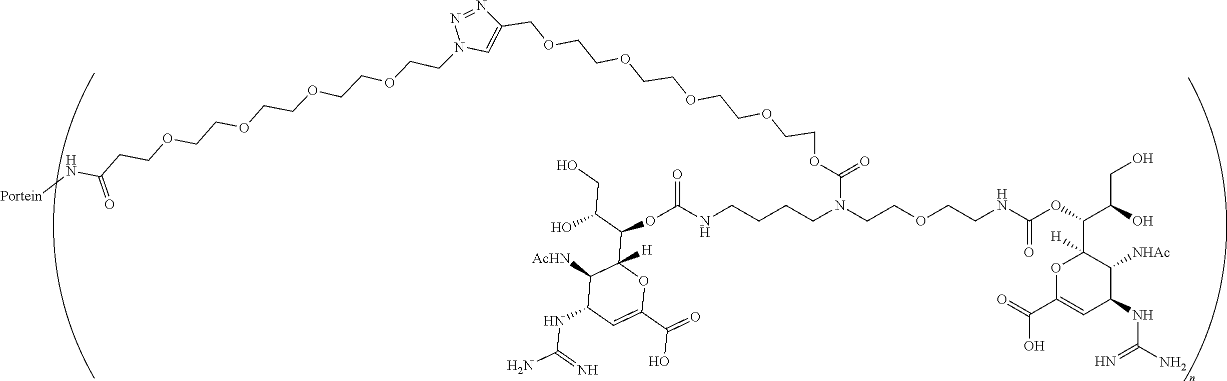 |
| C-75 | 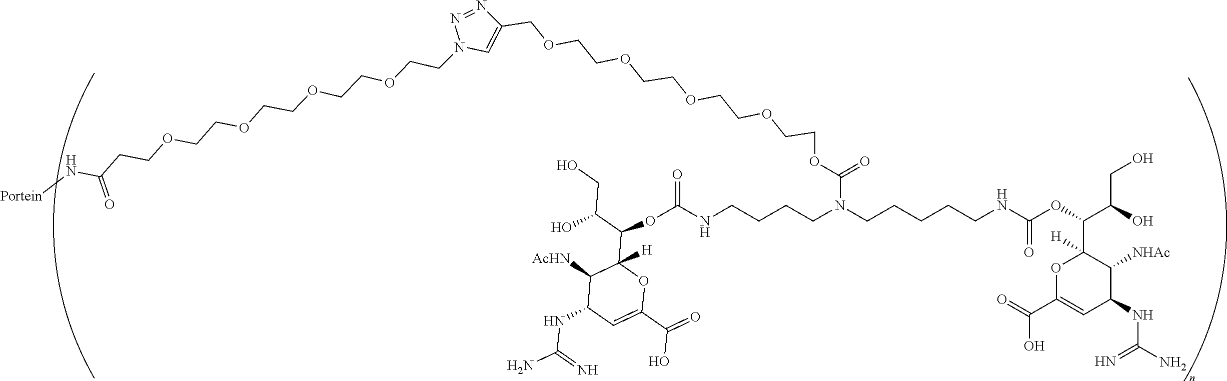 |
| C-76 | 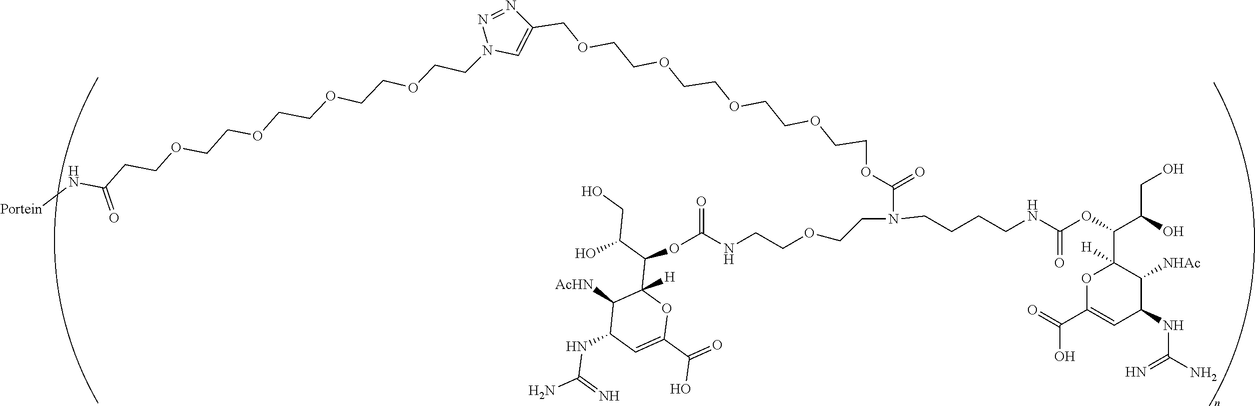 |
| C-77 | 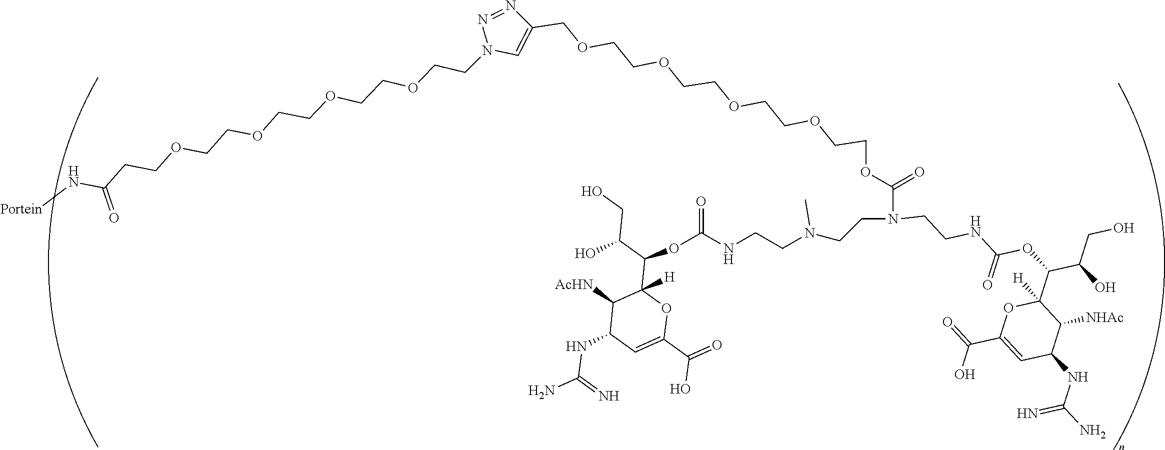 |
| C-78 | 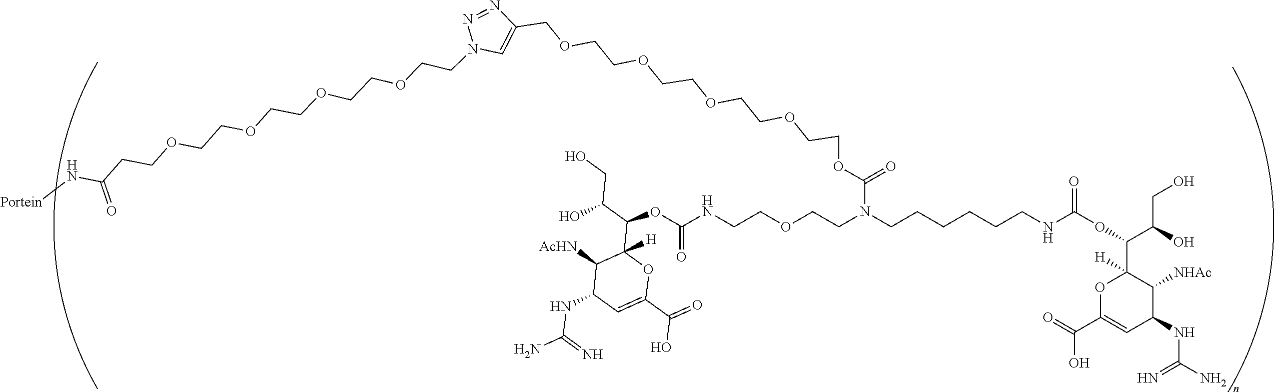 |
| C-79 | 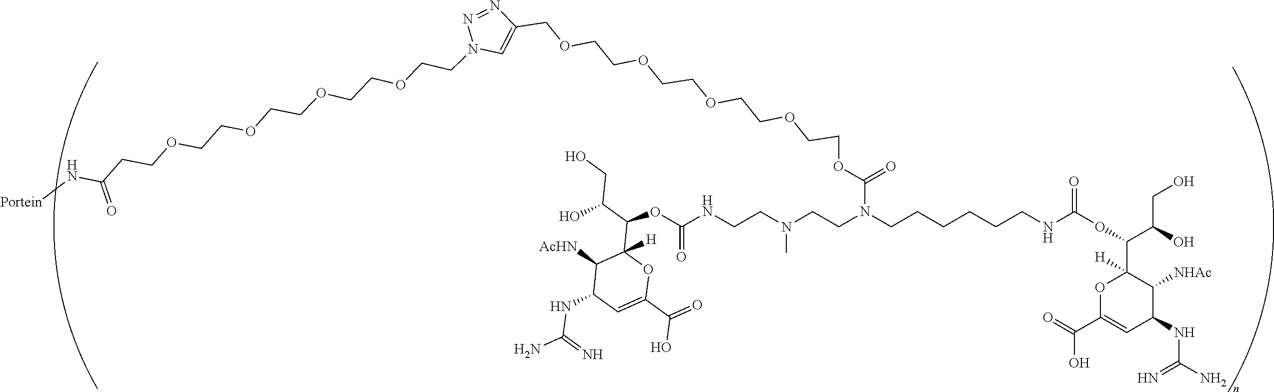 |
| C-80 | 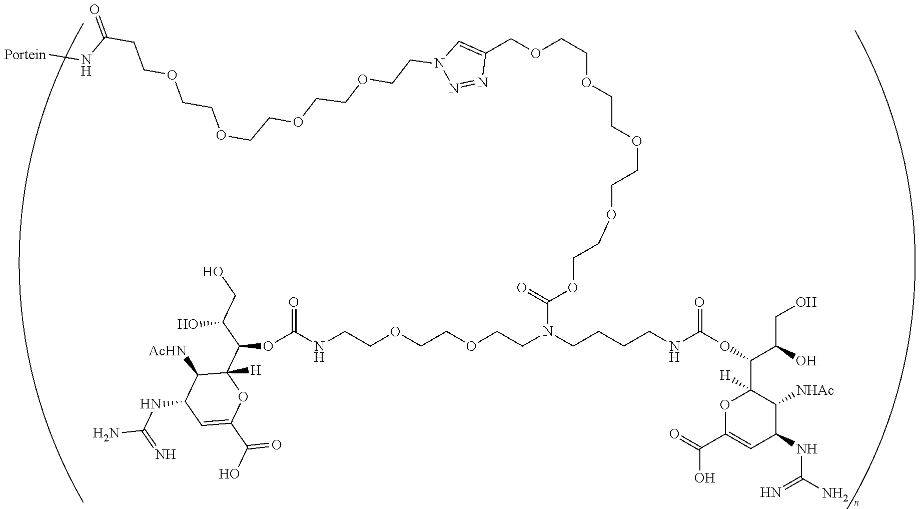 |
| C-81 | 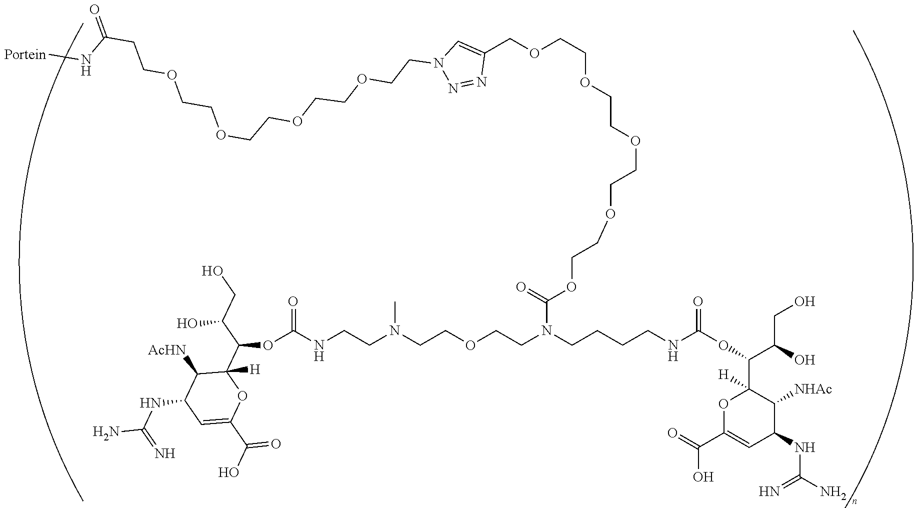 |
| C-82 | 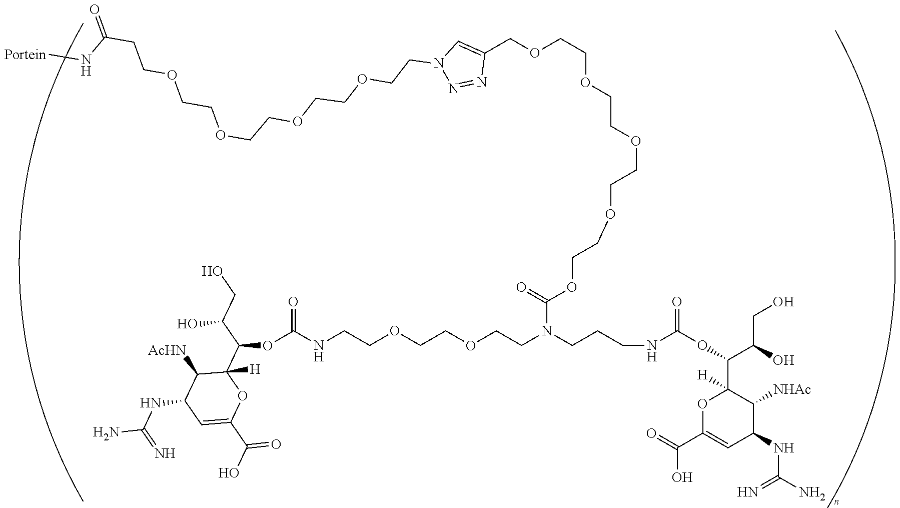 |
| C-83 | 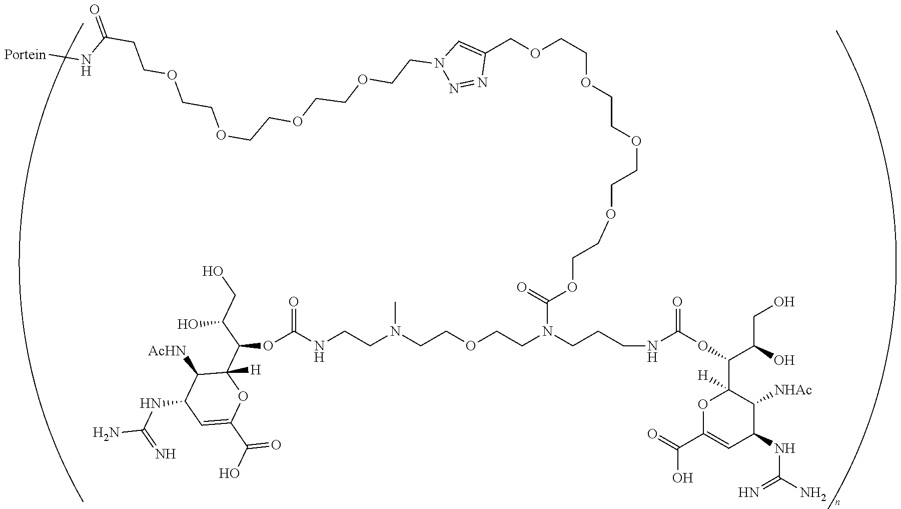 |
| C-84 | 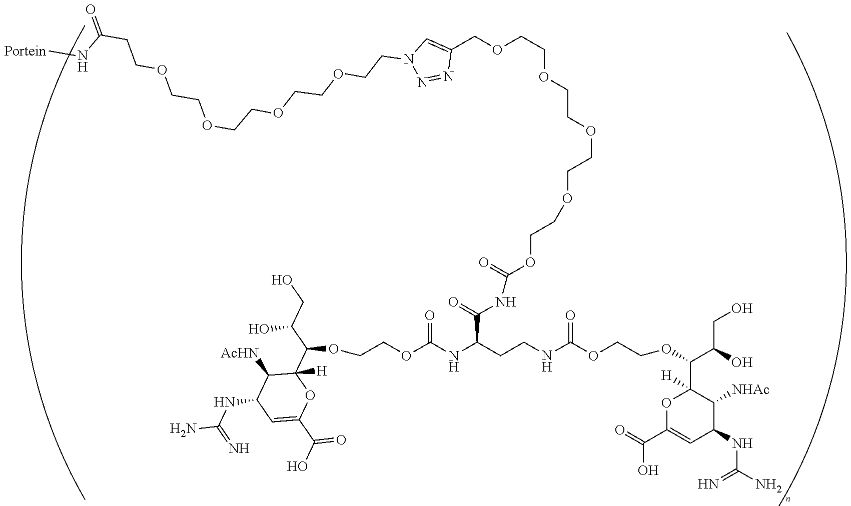 |
| C-85 | 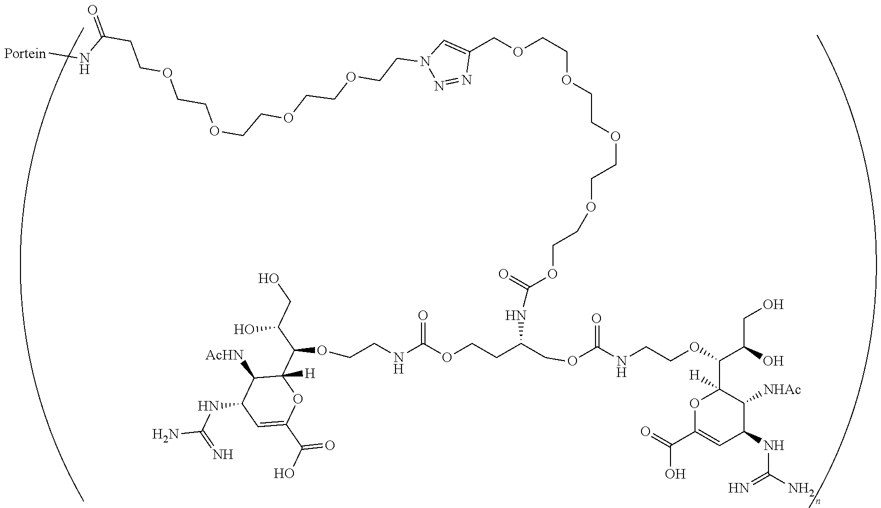 |
| C-86 | 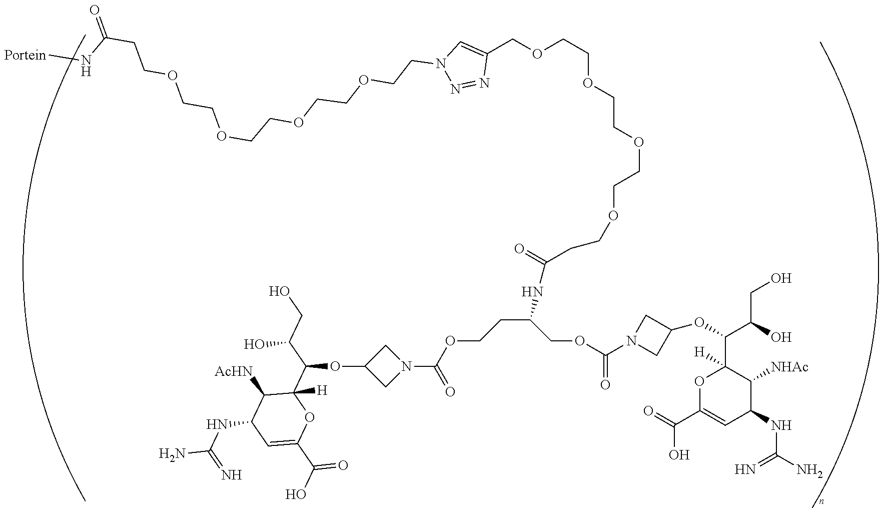 |
| C-87 | 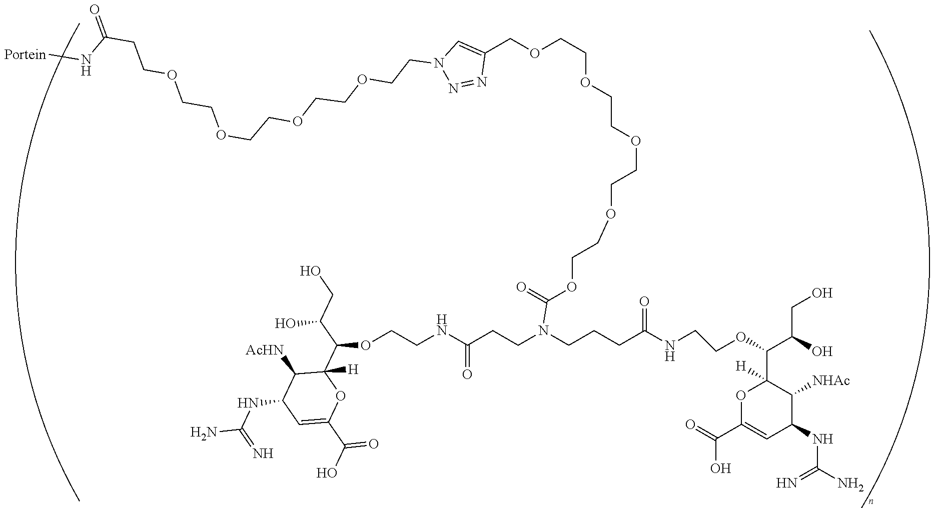 |
| C-88 | 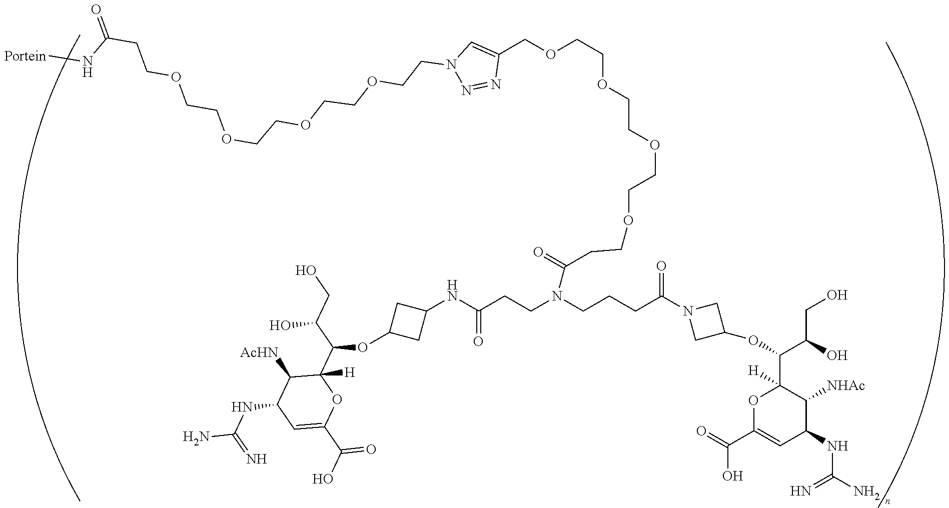 |
| C-89 | 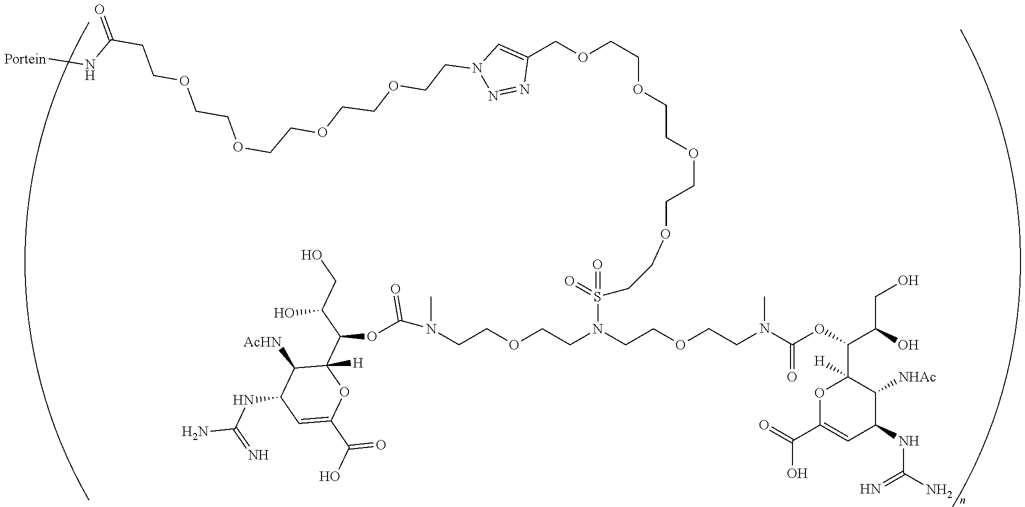 |
| C-90 | 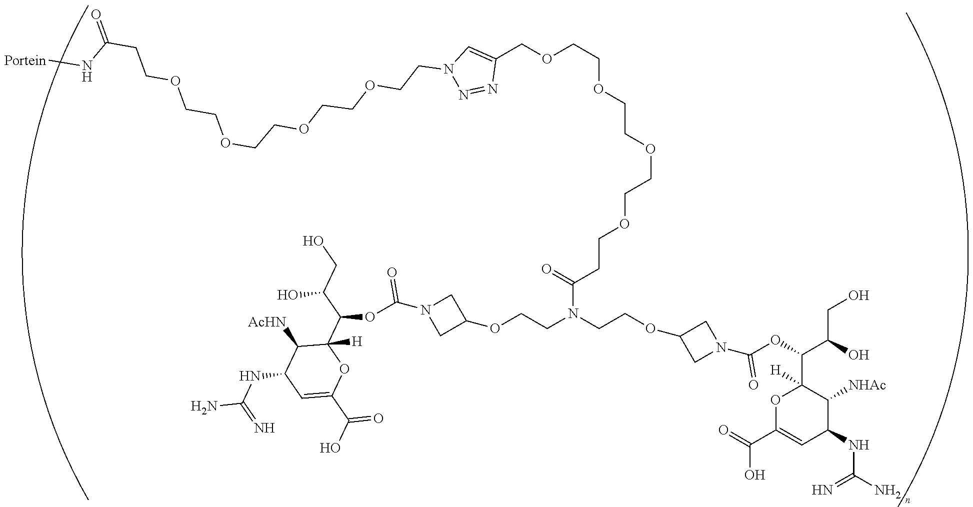 |
| C-91 | 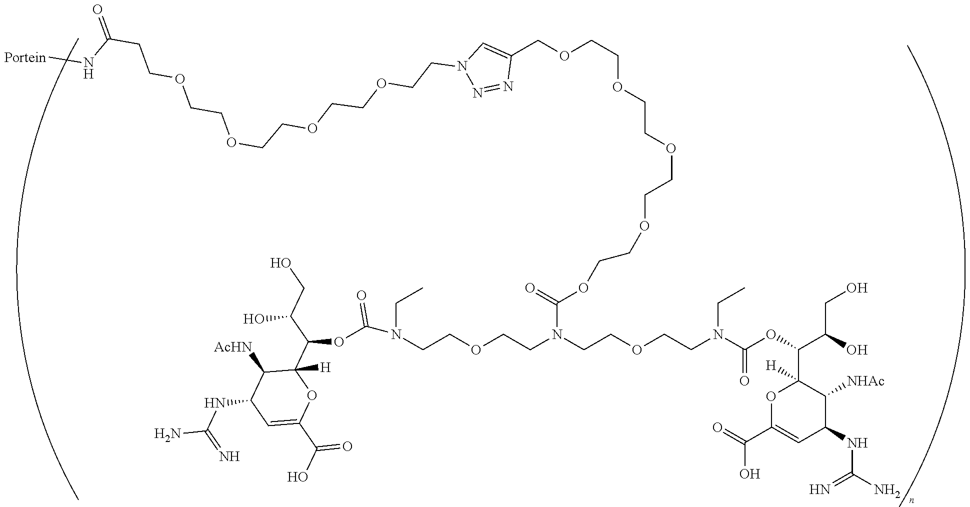 |
| C-92 | 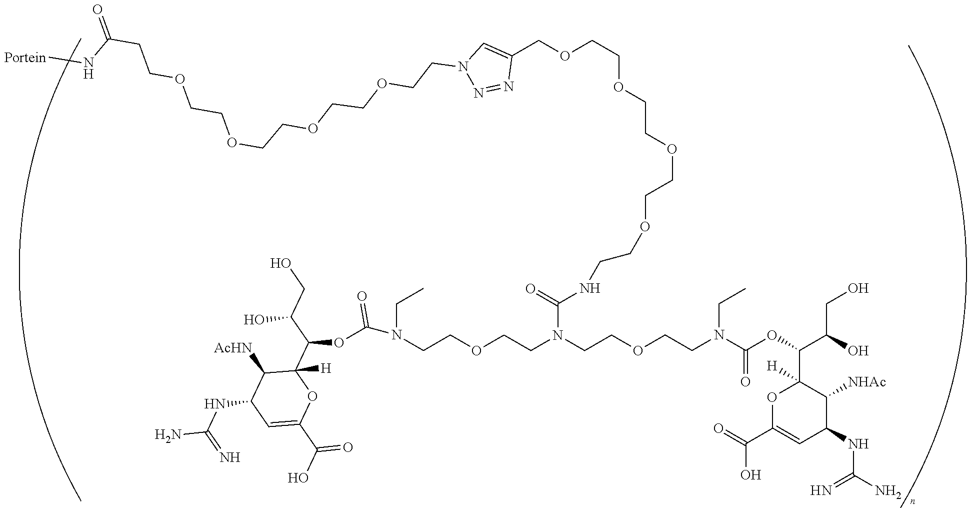 |
| C-93 | 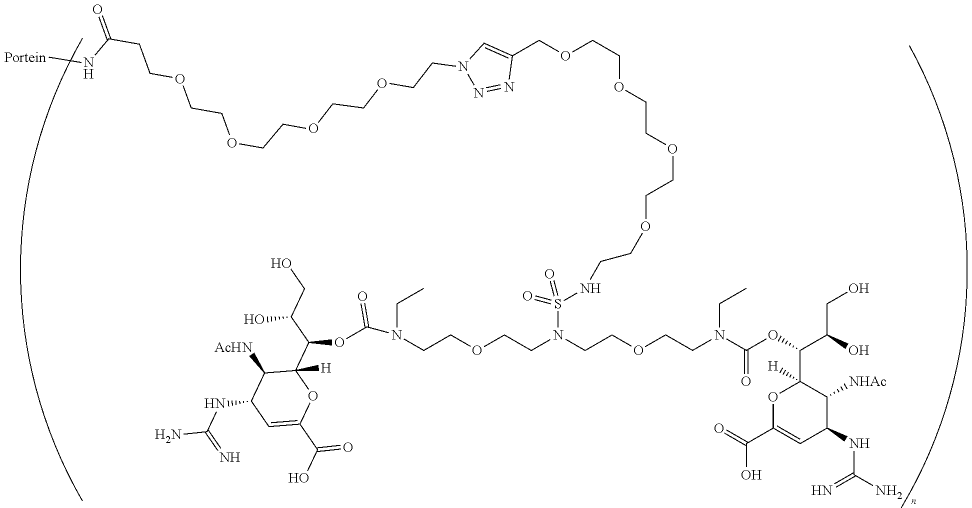 |
| C-94 | 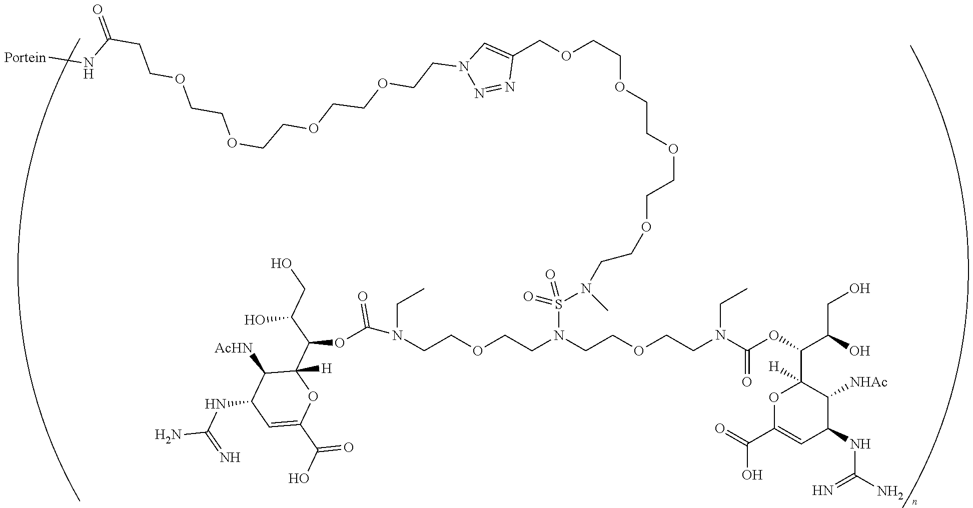 |
| C-95 | 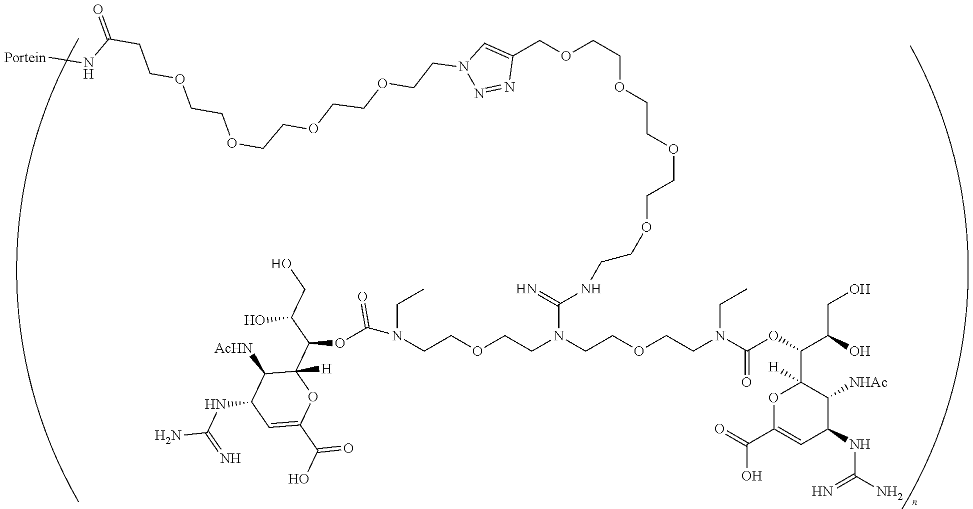 |
| C-96 | 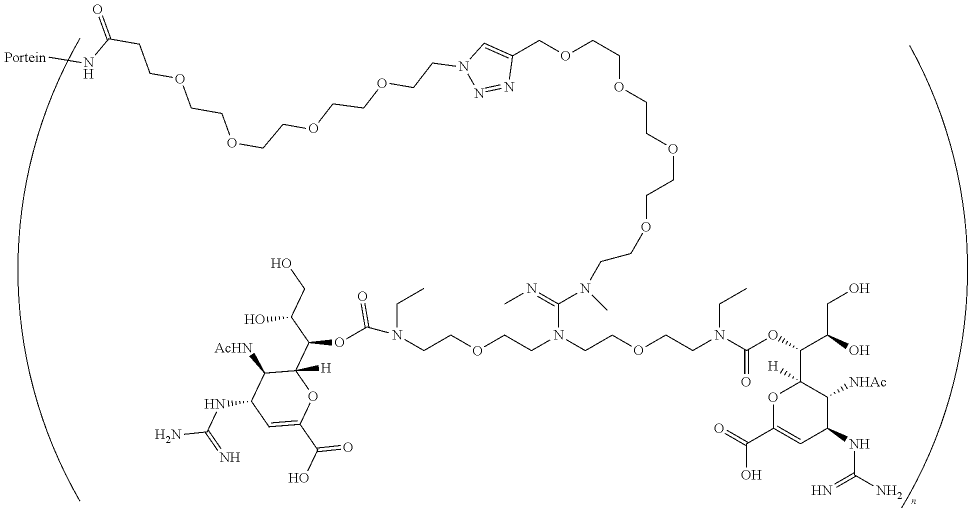 |
| C-97 | 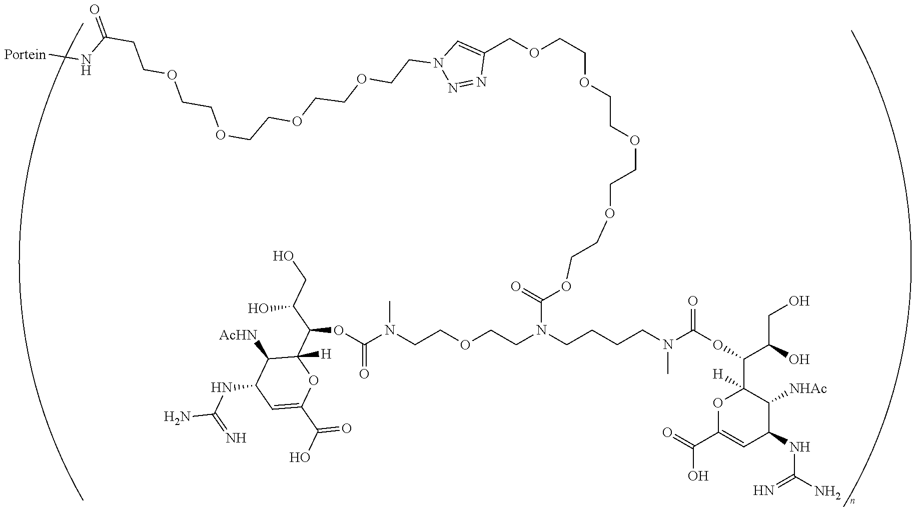 |
| C-98 | 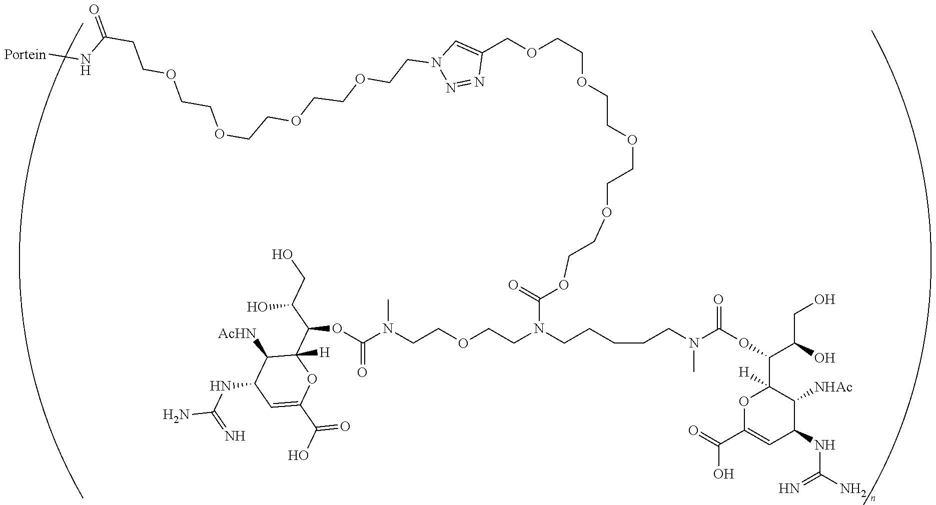 |
| C-99 | 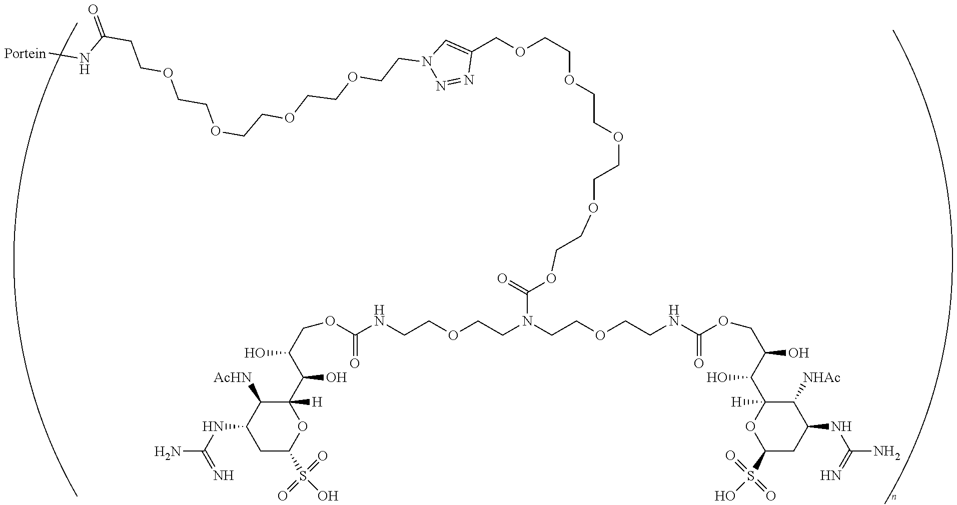 |
| C-100 | 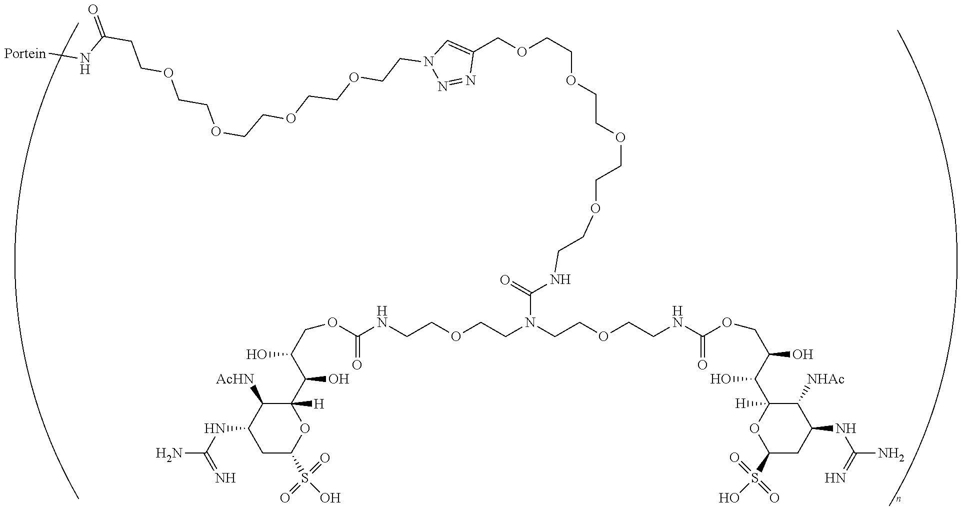 |
| C-101 | 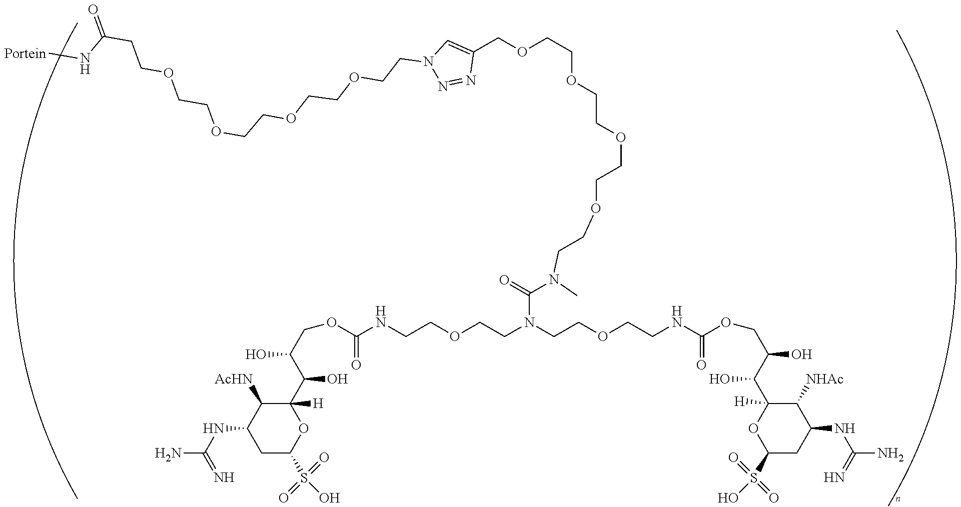 |
| C-102 | 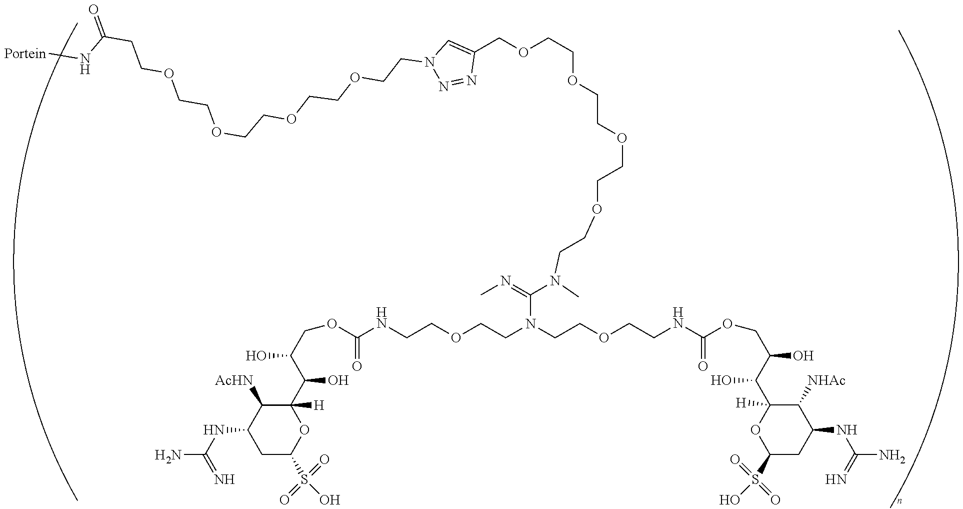 |
| C-103 | 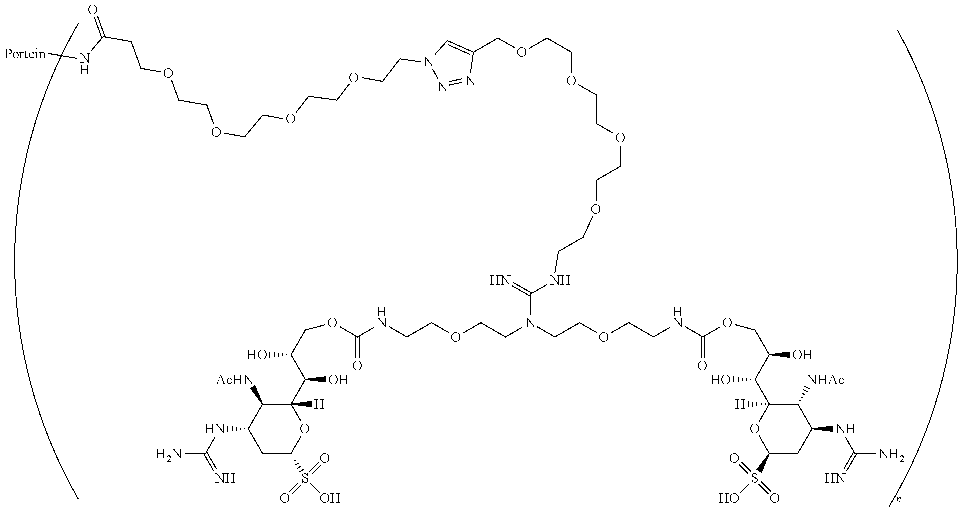 |
| C-104 | 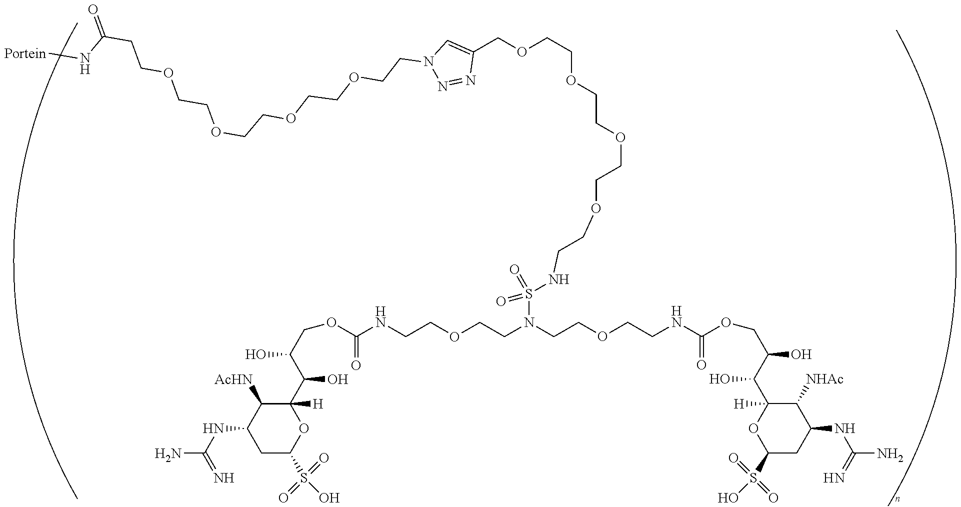 |
| C-105 | 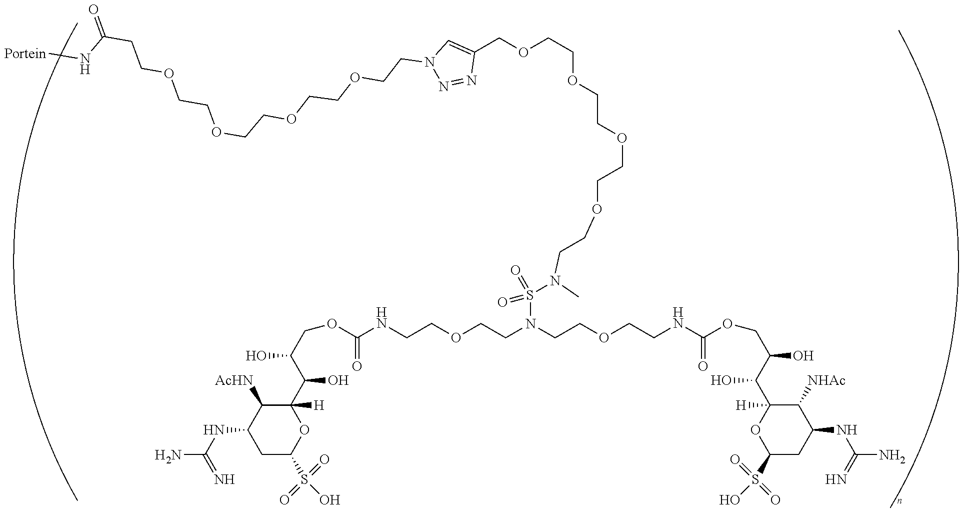 |
| C-106 | 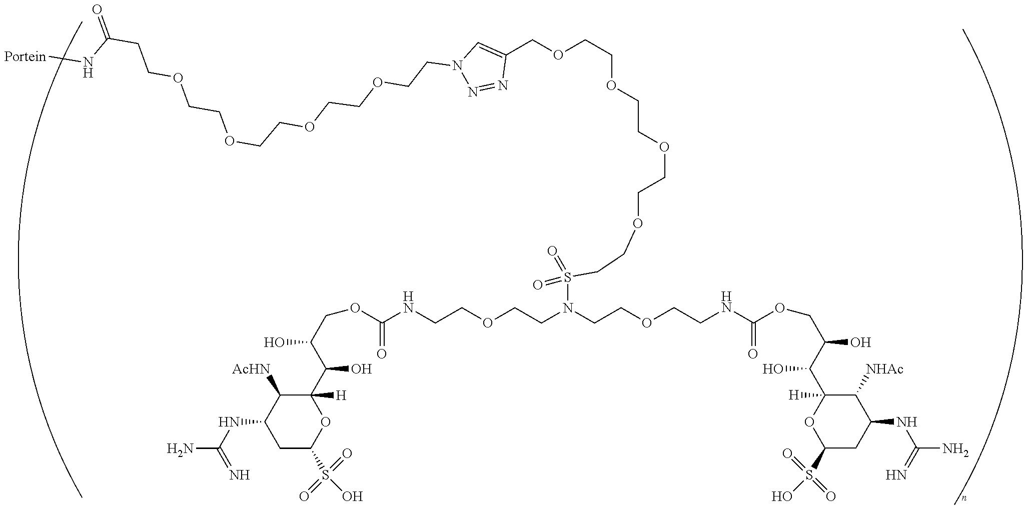 |
| C-107 | 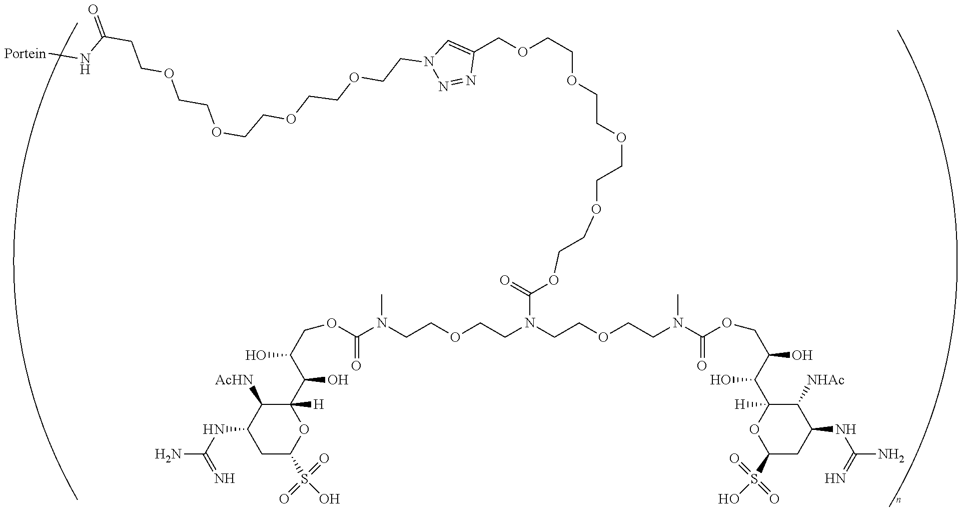 |
| C-108 | 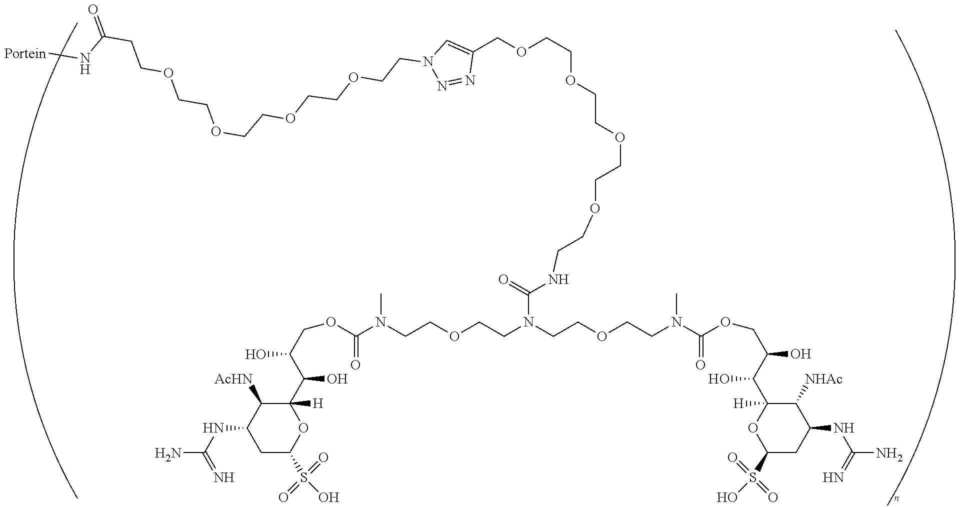 |
| C-109 | 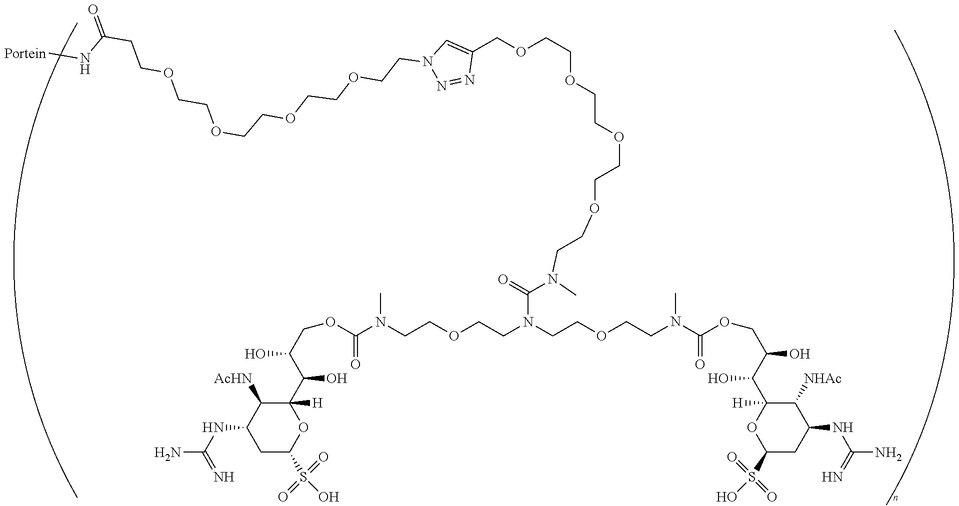 |
| C-110 | 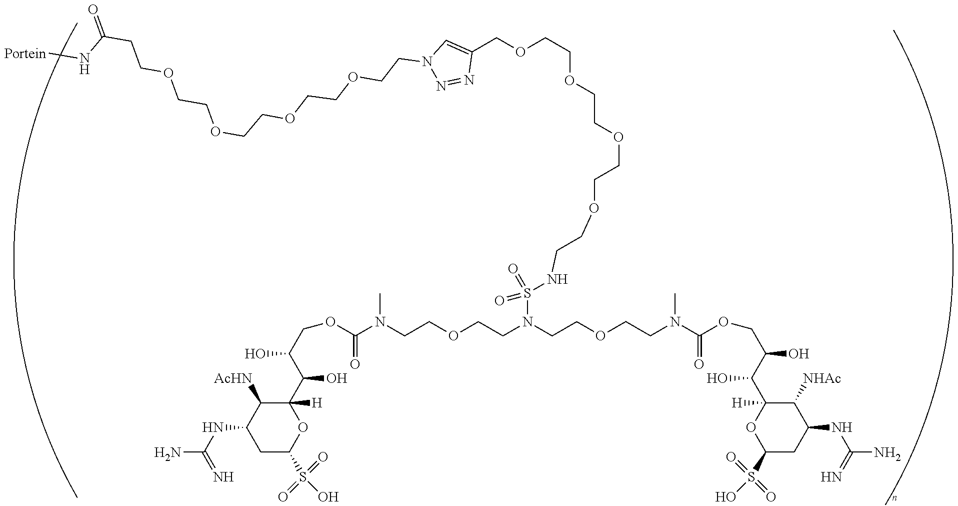 |
| C-111 | 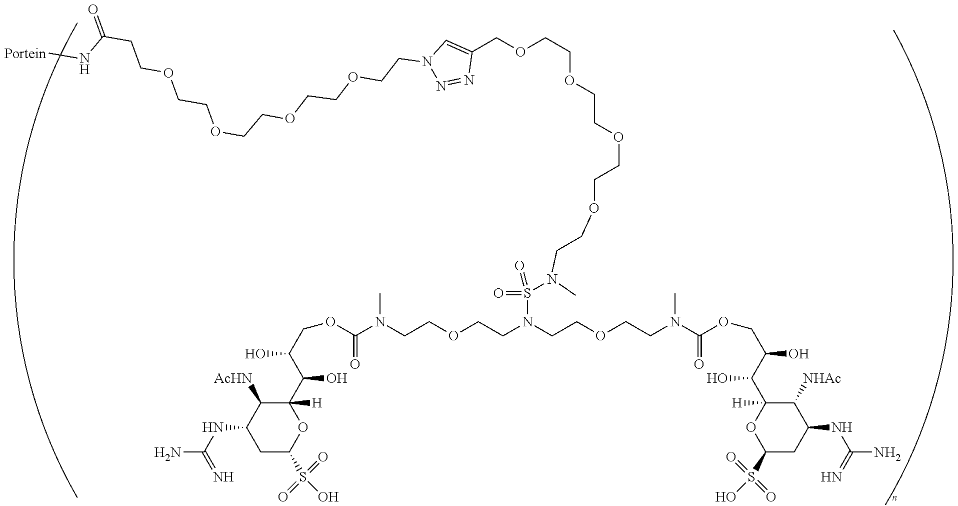 |
| C-112 | 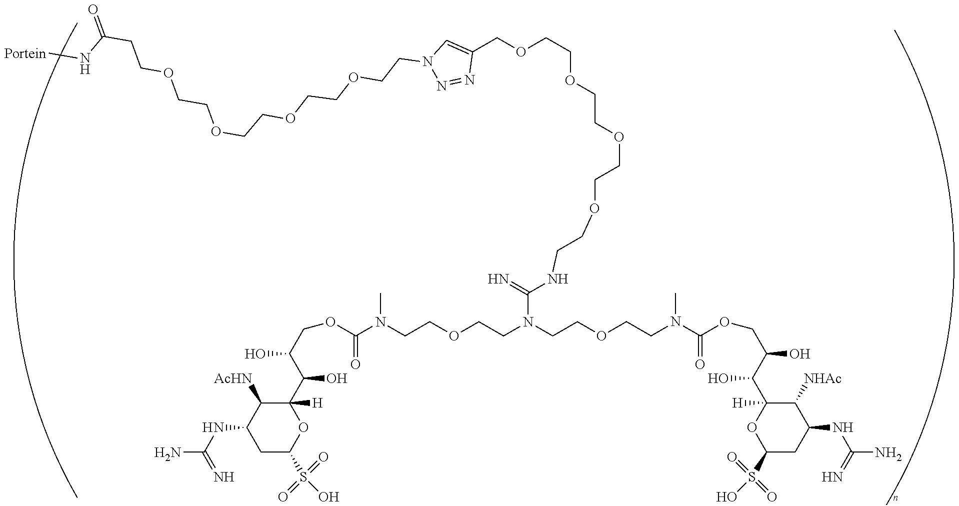 |
| C-113 | 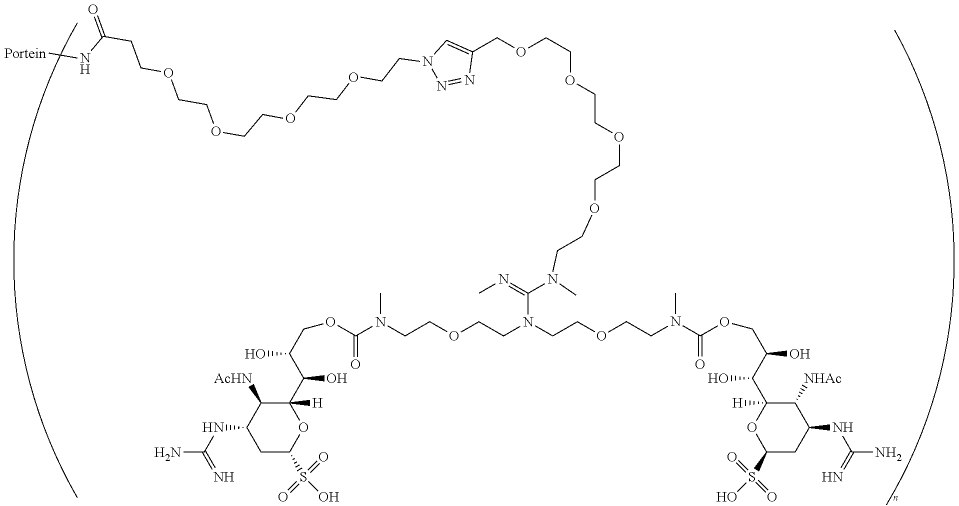 |
| C-114 | 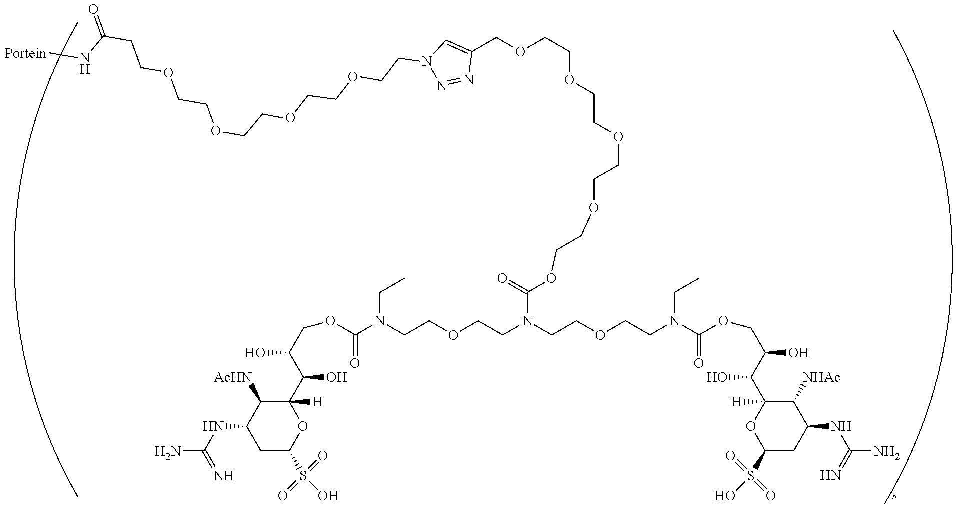 |
| C-115 | 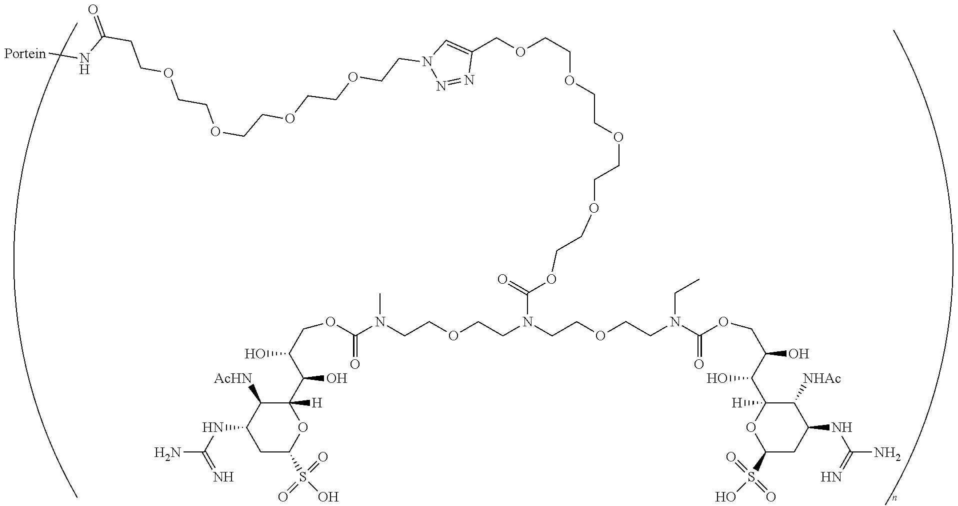 |

where n is as defined in claim 1, and protein is E as defined in claim 1.

6. The conjugate of claim 1, or a pharmaceutically acceptable salt, ester, isomer, solvate, prodrug or isotopically labeled derivative thereof, wherein a ratio of n to m is from 1 to 20, preferably from 2 to 10, such as 2, 3, 4, 5, 6, 7, 8, 9 or 10.

7. The conjugate of claim 1, or a pharmaceutically acceptable salt, ester, isomer, solvate, prodrug or isotopically labeled derivative thereof, wherein the conjugate has an average DAR value between 0.5 and 10.0.

8. The conjugate of claim 1, or a pharmaceutically acceptable salt, ester, isomer, solvate, prodrug or isotopically labeled derivative thereof, wherein E comprises an Fc structural domain monomer or an Fc structural domain containing said Fc structural domain monomer, wherein said Fc structural domain monomer comprises or consists of an amino acid sequence of any one of SEQ ID Nos. 1-68 or an amino acid sequence that is at least 95% identical to any one of SEQ ID Nos. 1-68.

9. The conjugate of claim 1, or a pharmaceutically acceptable salt, ester, isomer, solvate, prodrug or isotopically labeled derivative thereof, wherein E is recognizable for viral surface antigens, such as CR6261, CR8020, MEDI8897, Palivizumab, SD38, or
the Fc structural domain monomer may be an Fc structural domain monomer of an antibody subtype (such as, IGHG1*01 (such as G1m(za)), IGHG1*07 (such as G1m(zax)), IGHG1*04(such as G1m(zav)), IGHG1*03(G1m(f)), IGHG1*08 (such as G1m(fa)), IGHG2*01, IGHG2*02, IGHG2*06, IGHG3*01, IGHG3*04, IGHG3*05, IGHG3*09, IGHG3*10, IGHG3*11, IGHG3*12, IGHG3*06, IGHG3*07, IGHG3*08, IGHG3*13, IGHG3*03, IGHG3*14, IGHG3*15, IGHG3*16, IGHG3*17, IGHG3*18, IGHG3*19, IGHG2*04, IGHG4*01, IGHG4*02, IGHG4*03) of any kind of immunoglobulins.

10. The conjugate of claim 1, or a pharmaceutically acceptable salt, ester, isomer, solvate, prodrug or isotopically labeled derivative thereof, wherein the conjugate has a structure below: 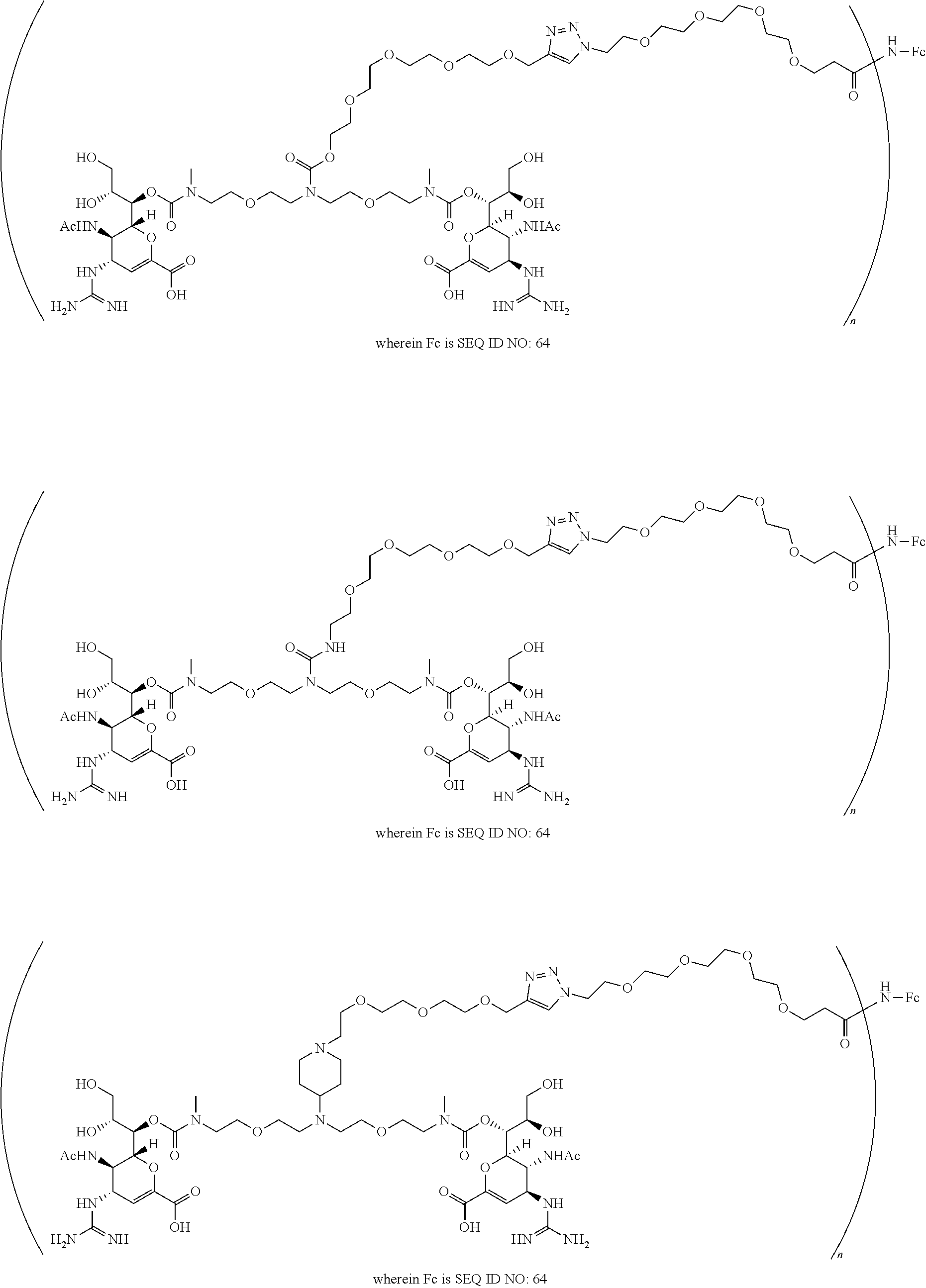 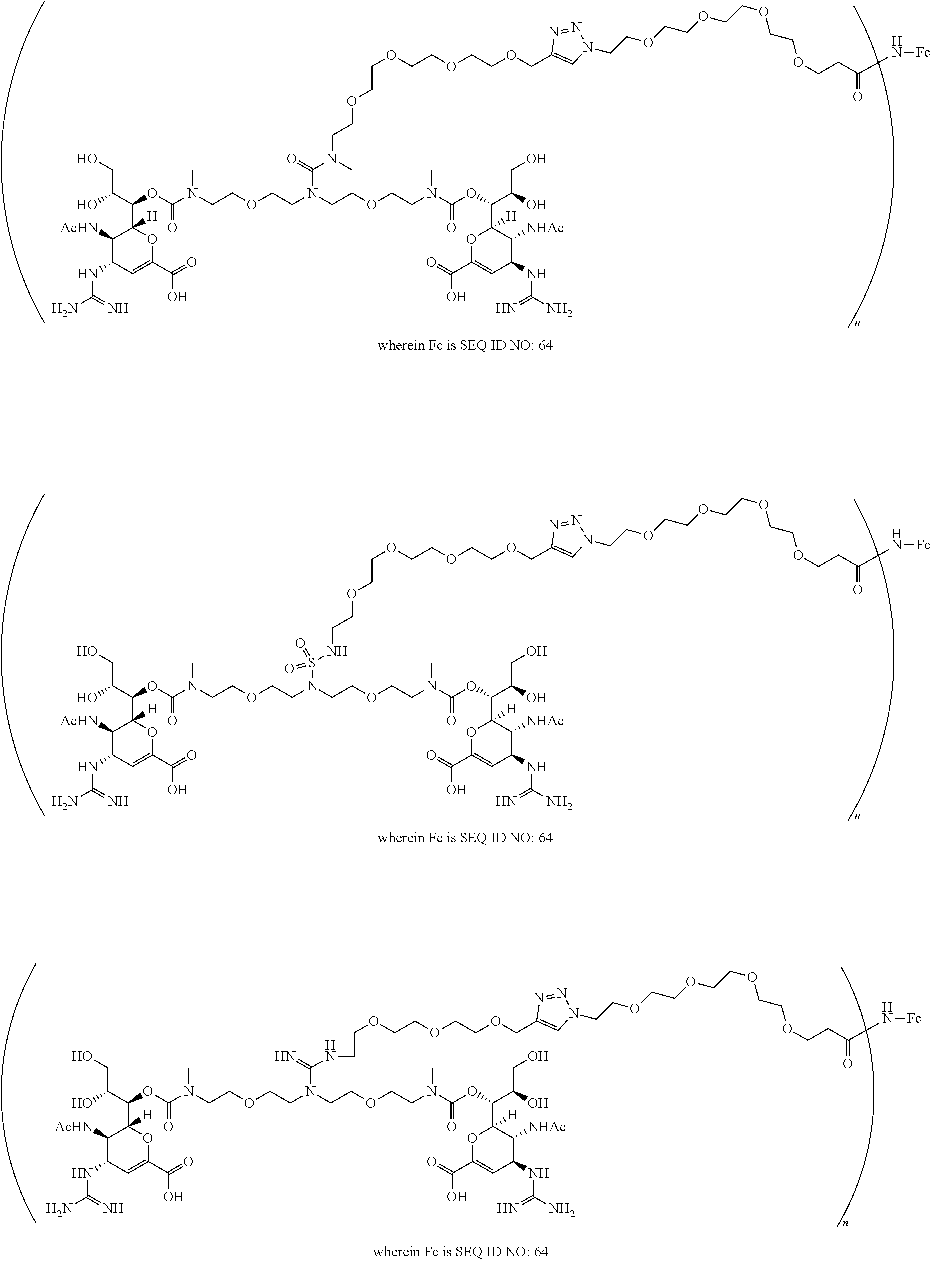 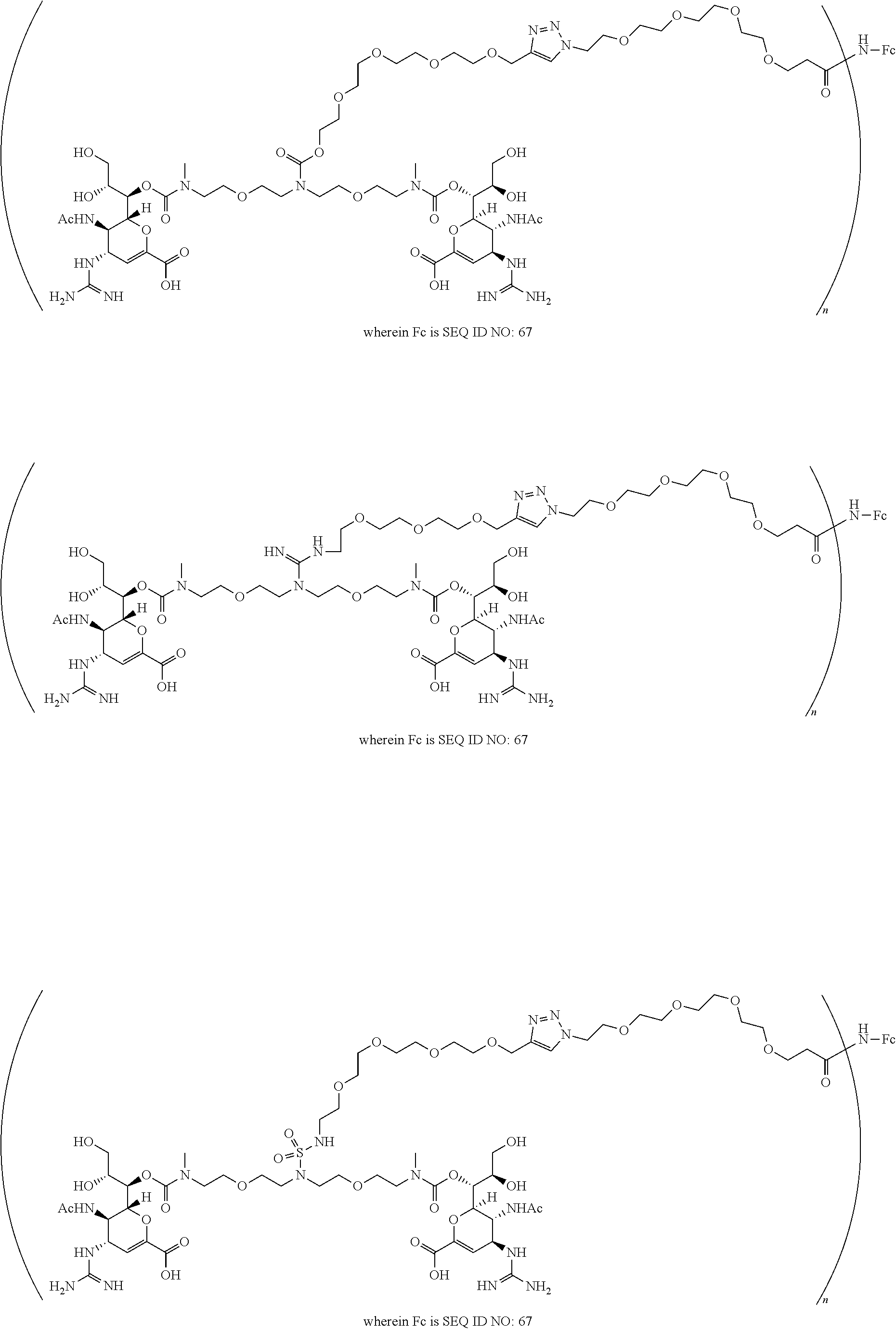
wherein n is as defined in claim 1.

11. A compound of formula I-2, or a pharmaceutically acceptable salt, ester, isomer, solvate, prodrug or isotopically labeled derivative thereof, 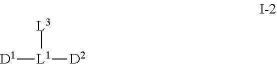
wherein L¹, D¹ and D² are as defined in formula I-1 of claim 1,
L³ is selected from following structures:

| L³ | |
|---|---|
| structure | |
| No. | Structure |
| L3-1 | 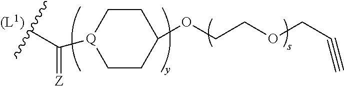 |
| L3-2 | 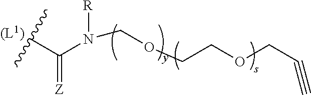 |
| L3-3 | 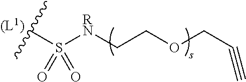 |
| L3-4 | 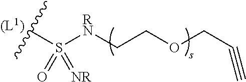 |
| L3-5 | 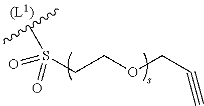 |
| L3-6 | 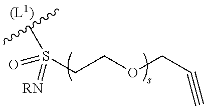 |
| L3-7 | 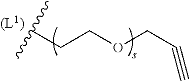 |
| L3-8 | 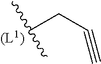 [SMILES: *CC#C] |
| L3-9 | 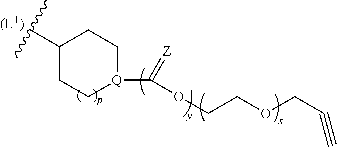 |
| L3-10 | 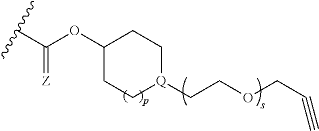 |
| L3-11 | 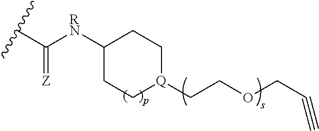 |
| L3-12 | 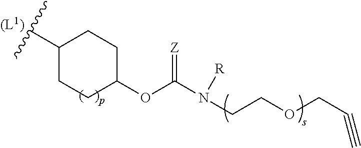 |
| L3-13 | 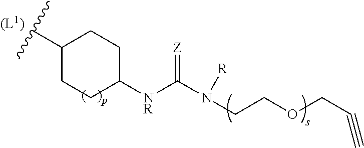 |
| L3-14 | 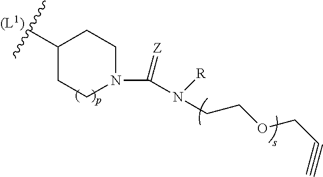 |
| L3-15 | 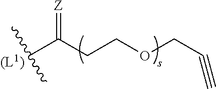 |

wherein each variable such as Z, R, Q, s, y, p is as defined for L² in claim 1.

12. The compound of Formula I-2 of claim 11, or a pharmaceutically acceptable salt, ester, isomer, solvate, prodrug or isotopically labeled derivative thereof, wherein said compound has a structure of Formula C-Inter-1 to C-Inter-115:

| No. | Structure |
|---|---|
| C-Inter-1 | 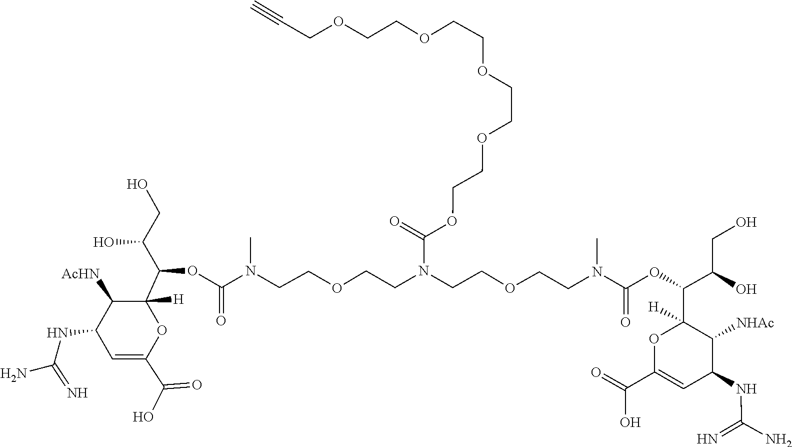 [SMILES: C#CCOCCOCCOCCOCCOC(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-2 | 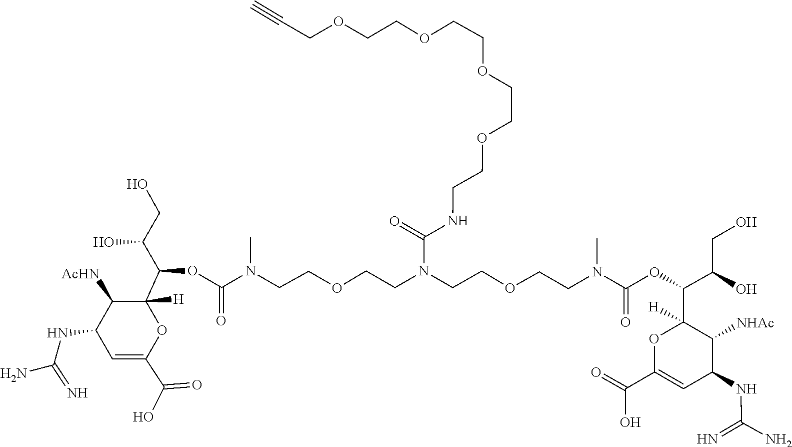 [SMILES: C#CCOCCOCCOCCOCCNC(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-3 | 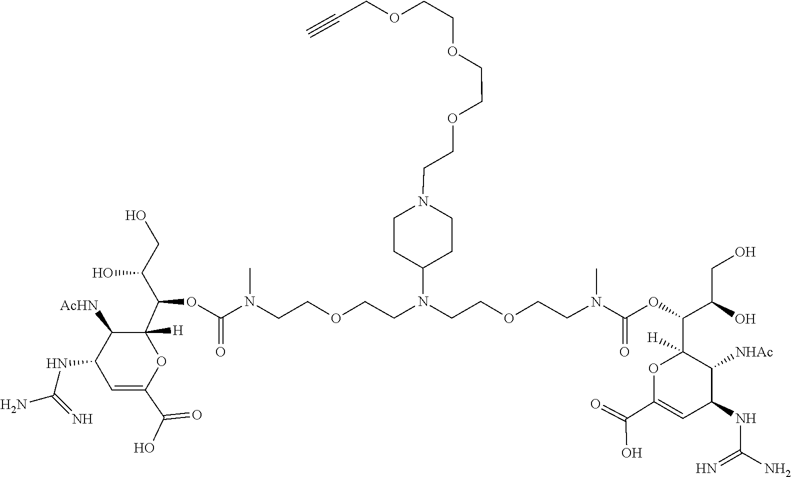 [SMILES: C#CCOCCOCCOCCN1CCC(N(CCOCCN(C)C(=O)O[C@@H]([C@@H]2OC(C(=O)O)=C[C@H](NC(=N)N)[C@H]2NC(C)=O)[C@H](O)CO)CCOCCN(C)C(=O)O[C@@H]([C@@H]2OC(C(=O)O)=C[C@H](NC(=N)N)[C@H]2NC(C)=O)[C@H](O)CO)CC1] |
| C-Inter-4 | 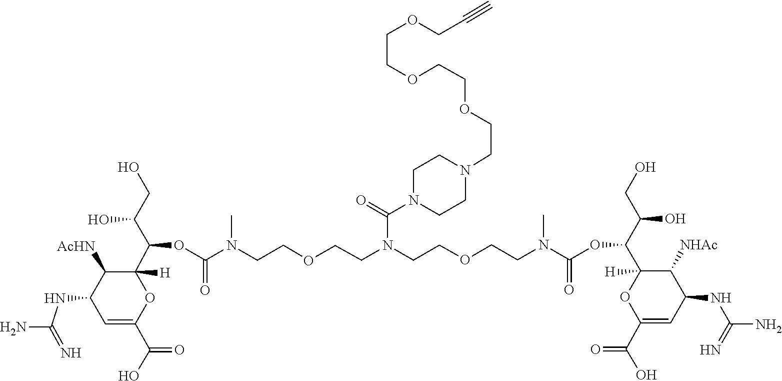 [SMILES: C#CCOCCOCCOCCN1CCN(C(=O)N(CCOCCN(C)C(=O)O[C@@H]([C@@H]2OC(C(=O)O)=C[C@H](NC(=N)N)[C@H]2NC(C)=O)[C@H](O)CO)CCOCCN(C)C(=O)O[C@@H]([C@@H]2OC(C(=O)O)=C[C@H](NC(=N)N)[C@H]2NC(C)=O)[C@H](O)CO)CC1] |
| C-Inter-5 | 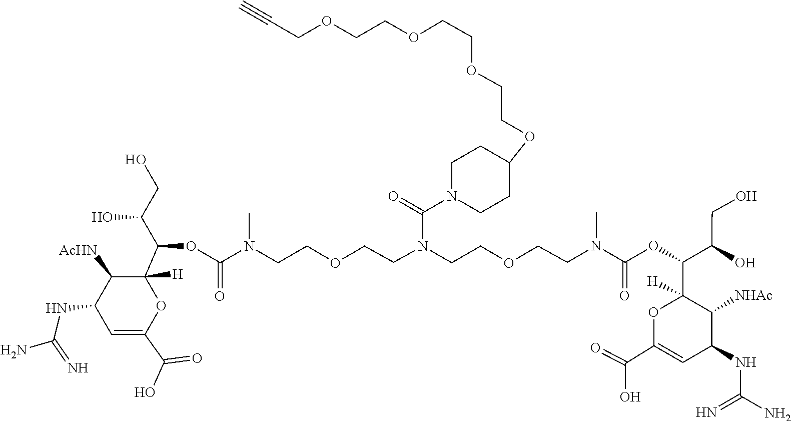 [SMILES: C#CCOCCOCCOCCOC1CCN(C(=O)N(CCOCCN(C)C(=O)O[C@@H]([C@@H]2OC(C(=O)O)=C[C@H](NC(=N)N)[C@H]2NC(C)=O)[C@H](O)CO)CCOCCN(C)C(=O)O[C@@H]([C@@H]2OC(C(=O)O)=C[C@H](NC(=N)N)[C@H]2NC(C)=O)[C@H](O)CO)CC1] |
| C-Inter-6 | 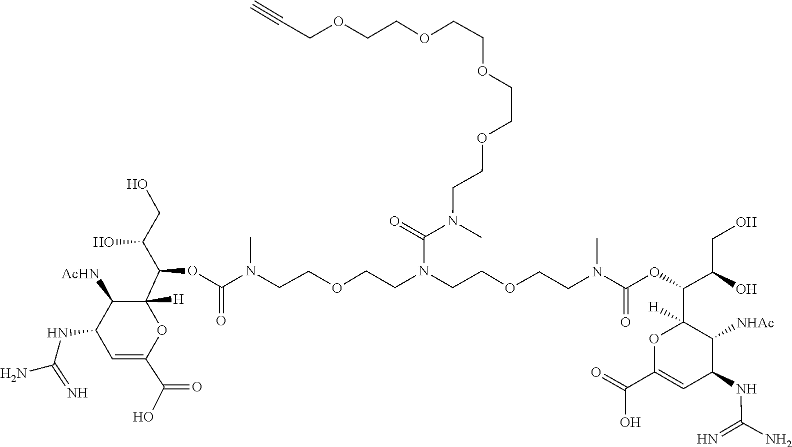 [SMILES: C#CCOCCOCCOCCOCCN(C)C(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-7 | 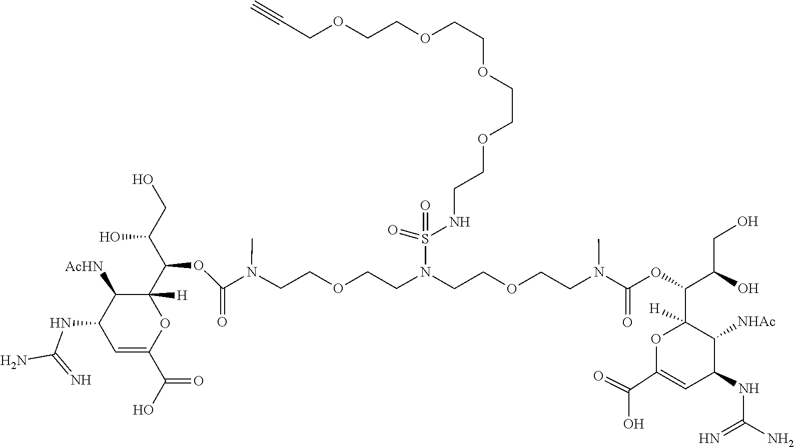 [SMILES: C#CCOCCOCCOCCOCCNS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-8 | 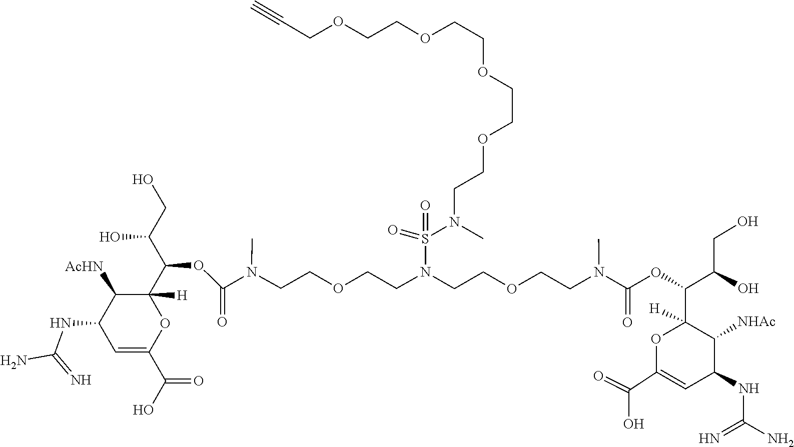 [SMILES: C#CCOCCOCCOCCOCCN(C)S(=O)(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-9 | 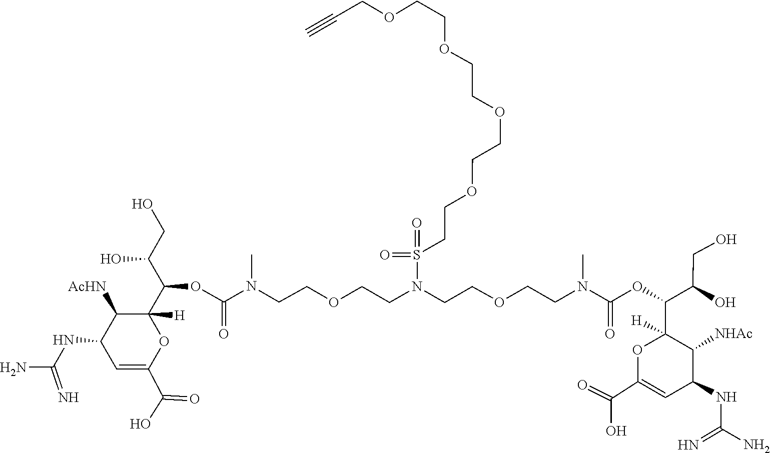 [SMILES: C#CCOCCOCCOCCOCCS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-10 | 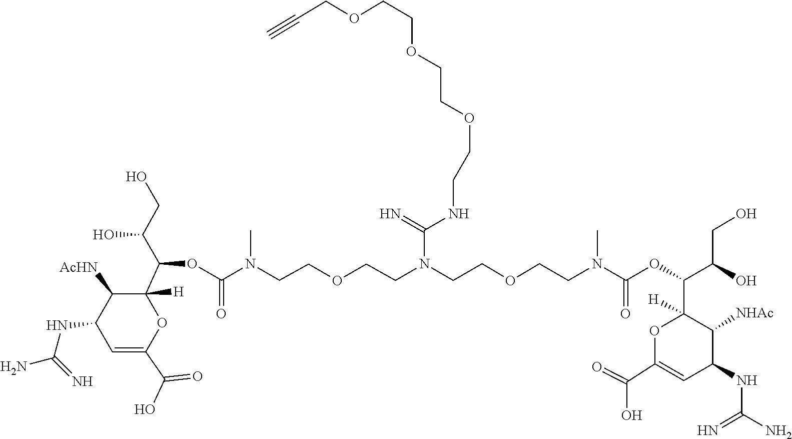 [SMILES: C#CCOCCOCCOCCNC(=N)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-11 | 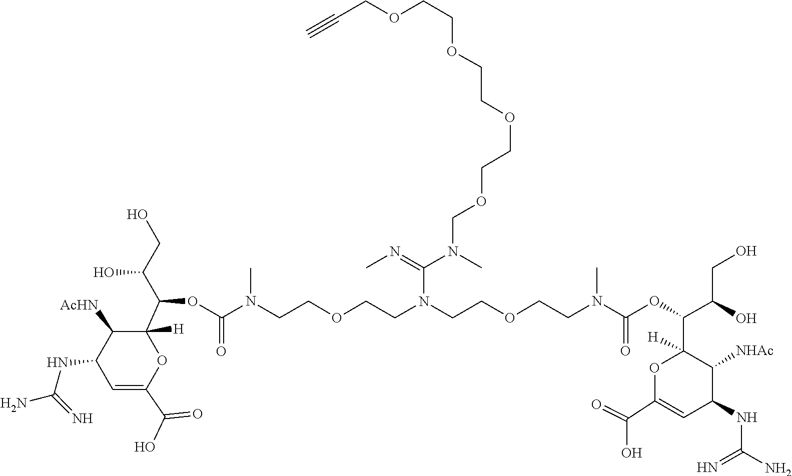 [SMILES: C#CCOCCOCCOCCOCN(C)/C(=N/C)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-12 | 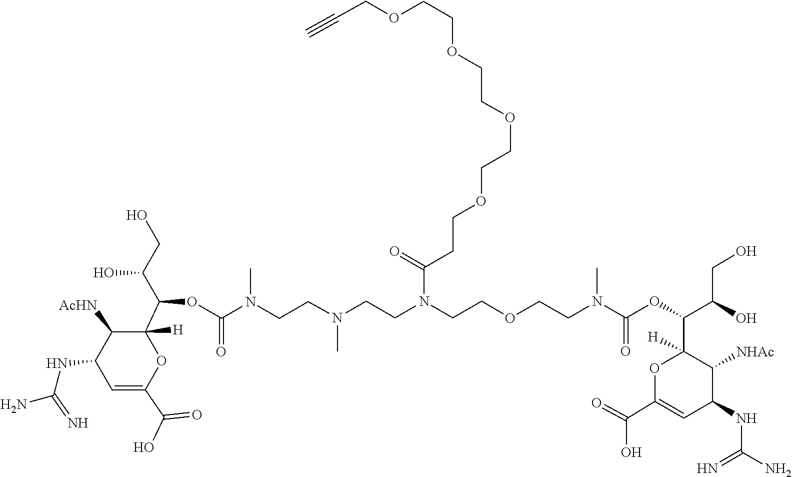 [SMILES: C#CCOCCOCCOCCOCCC(=O)N(CCOCCN(C)C(=O)O[C@@H]([C@@H]1OC(C(=O)O)=C[C@H](NC(=N)N)[C@H]1NC(C)=O)[C@H](O)CO)CCN(C)CCN(C)C(=O)O[C@@H]([C@@H]1OC(C(=O)O)=C[C@H](NC(=N)N)[C@H]1NC(C)=O)[C@H](O)CO] |
| C-Inter-13 | 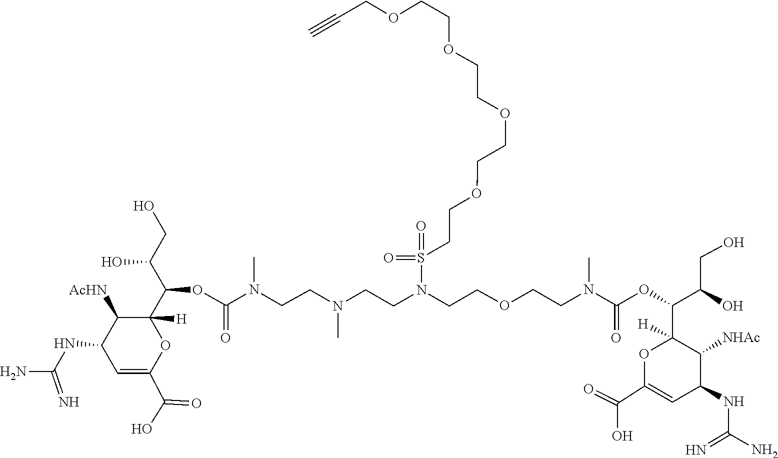 [SMILES: C#CCOCCOCCOCCOCCS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCN(C)CCN(C)C(=O)O[C@@H]([C@@H]1OC(C(=O)O)=C[C@H](NC(=N)N)[C@H]1NC(C)=O)[C@H](O)CO] |
| C-Inter-14 | 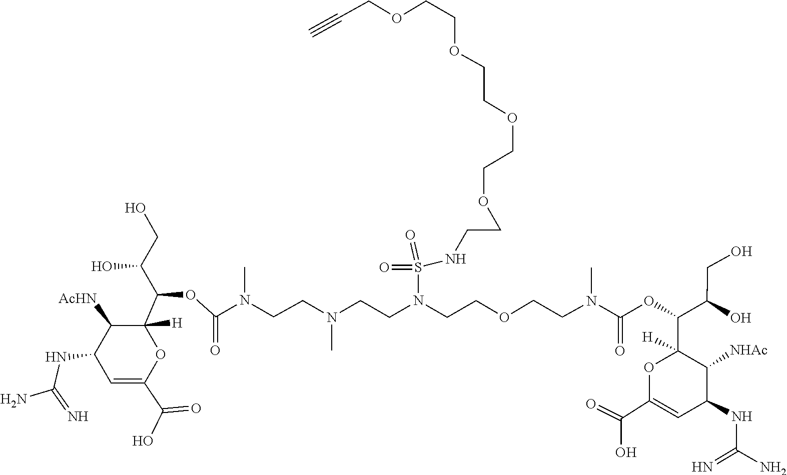 [SMILES: C#CCOCCOCCOCCOCCNS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCN(C)CCN(C)C(=O)O[C@@H]([C@@H]1OC(C(=O)O)=C[C@H](NC(=N)N)[C@H]1NC(C)=O)[C@H](O)CO] |
| C-Inter-15 | 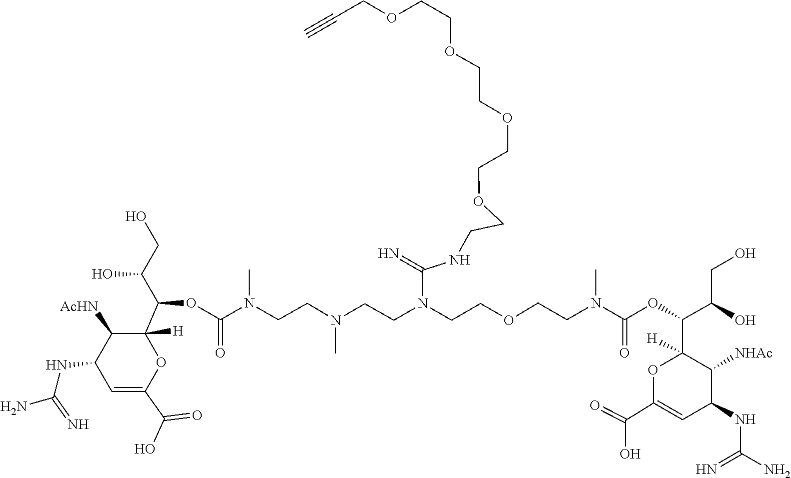 [SMILES: C#CCOCCOCCOCCOCCNC(=N)N(CCOCCN(C)C(=O)O[C@@H]([C@@H]1OC(C(=O)O)=C[C@H](NC(=N)N)[C@H]1NC(C)=O)[C@H](O)CO)CCN(C)CCN(C)C(=O)O[C@@H]([C@@H]1OC(C(=O)O)=C[C@H](NC(=N)N)[C@H]1NC(C)=O)[C@H](O)CO] |
| C-Inter-16 | 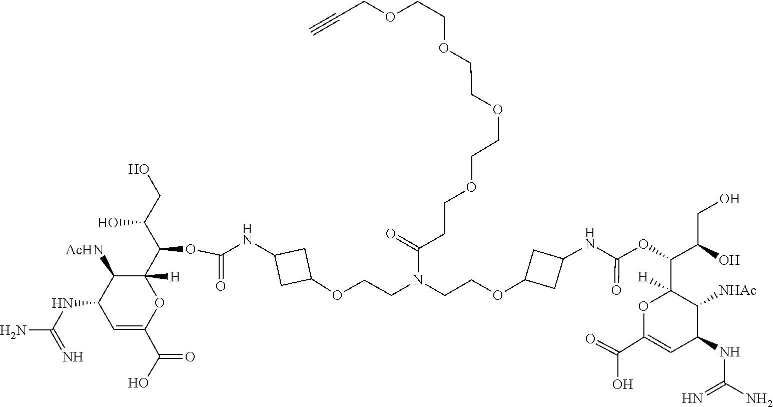 [SMILES: C#CCOCCOCCOCCOCCC(=O)N(CCOC1CC(NC(=O)O[C@@H]([C@@H]2OC(C(=O)O)=C[C@H](NC(=N)N)[C@H]2NC(C)=O)[C@H](O)CO)C1)CCOC1CC(NC(=O)O[C@@H]([C@@H]2OC(C(=O)O)=C[C@H](NC(=N)N)[C@H]2NC(C)=O)[C@H](O)CO)C1] |
| C-Inter-17 | 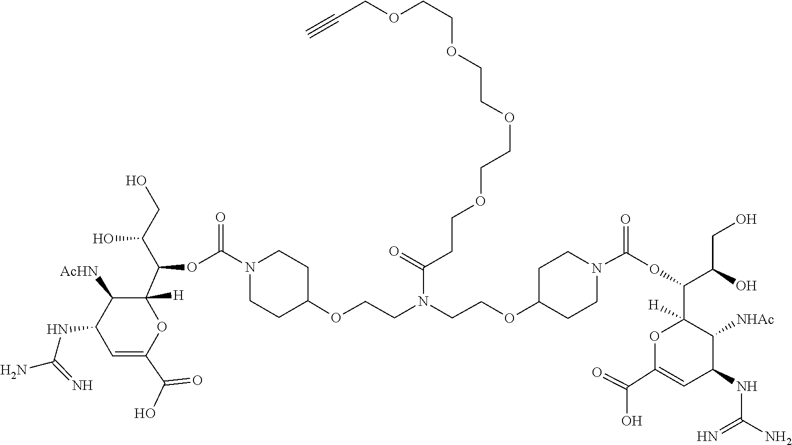 [SMILES: C#CCOCCOCCOCCOCCC(=O)N(CCOC1CCN(C(=O)O[C@@H]([C@@H]2OC(C(=O)O)=C[C@H](NC(=N)N)[C@H]2NC(C)=O)[C@H](O)CO)CC1)CCOC1CCN(C(=O)O[C@@H]([C@@H]2OC(C(=O)O)=C[C@H](NC(=N)N)[C@H]2NC(C)=O)[C@H](O)CO)CC1] |
| C-Inter-18 | 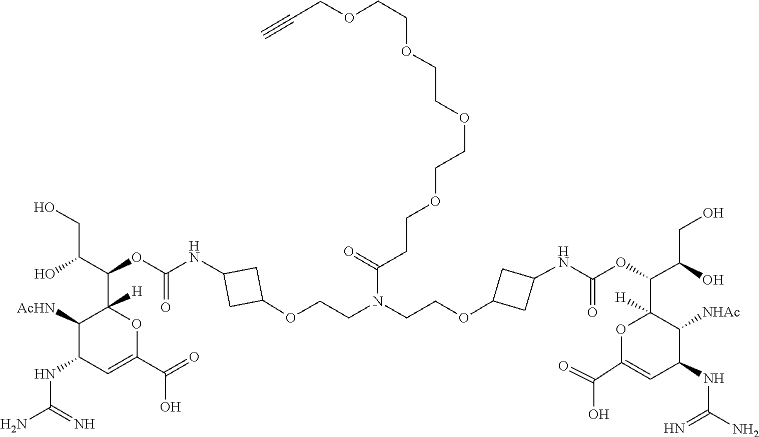 [SMILES: C#CCOCCOCCOCCOCCC(=O)N(CCOC1CC(NC(=O)O[C@@H]([C@@H]2OC(C(=O)O)=C[C@H](NC(=N)N)[C@H]2NC(C)=O)[C@H](O)CO)C1)CCOC1CC(NC(=O)O[C@@H]([C@@H]2OC(C(=O)O)=C[C@H](NC(=N)N)[C@H]2NC(C)=O)[C@H](O)CO)C1] |
| C-Inter-19 | 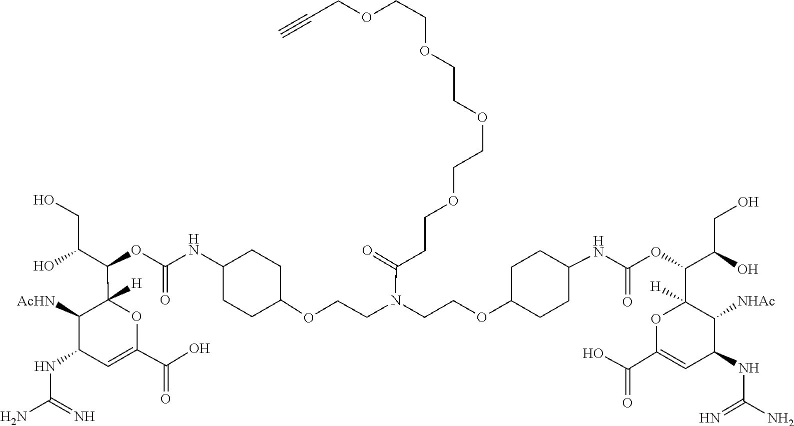 [SMILES: C#CCOCCOCCOCCOCCC(=O)N(CCOC1CCC(NC(=O)O[C@@H]([C@@H]2OC(C(=O)O)=C[C@H](NC(=N)N)[C@H]2NC(C)=O)[C@H](O)CO)CC1)CCOC1CCC(NC(=O)O[C@@H]([C@@H]2OC(C(=O)O)=C[C@H](NC(=N)N)[C@H]2NC(C)=O)[C@H](O)CO)CC1] |
| C-Inter-20 | 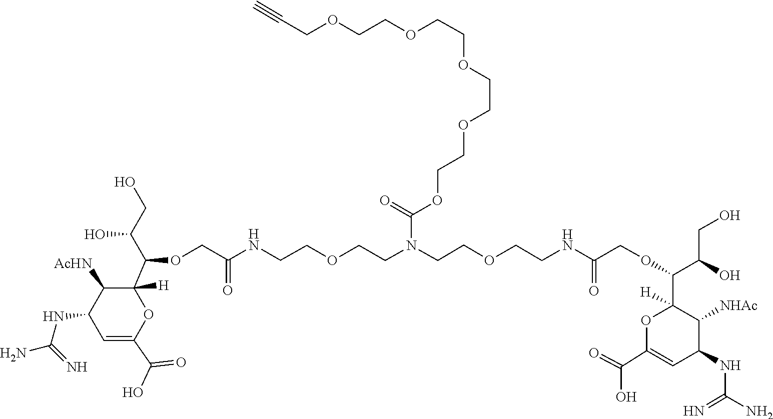 [SMILES: C#CCOCCOCCOCCOCCOC(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C-Inter-21 | 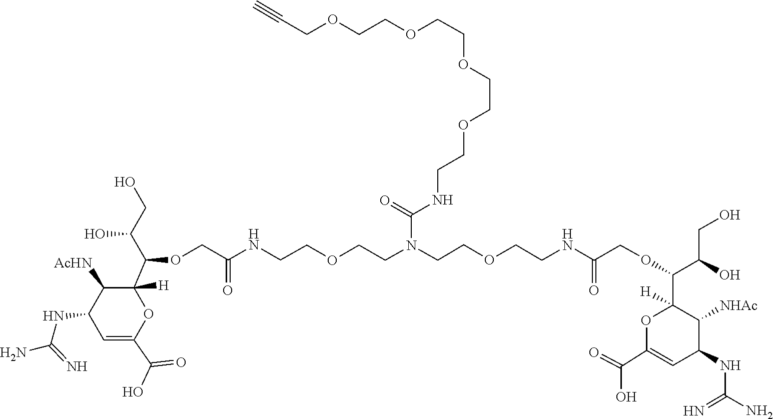 [SMILES: C#CCOCCOCCOCCOCCNC(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C-Inter-22 | 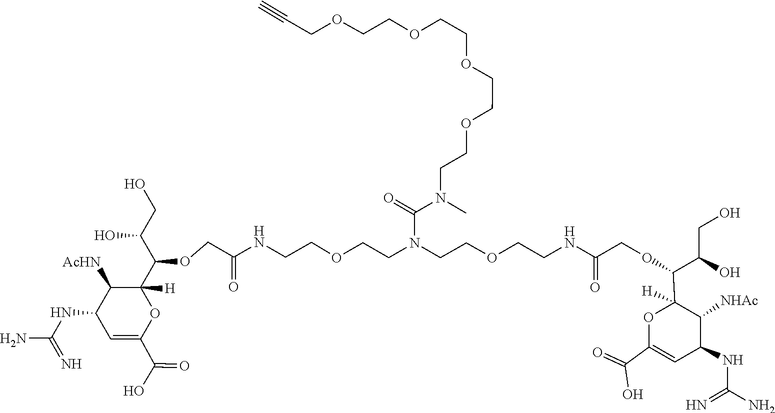 [SMILES: C#CCOCCOCCOCCOCCN(C)C(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C-Inter-23 | 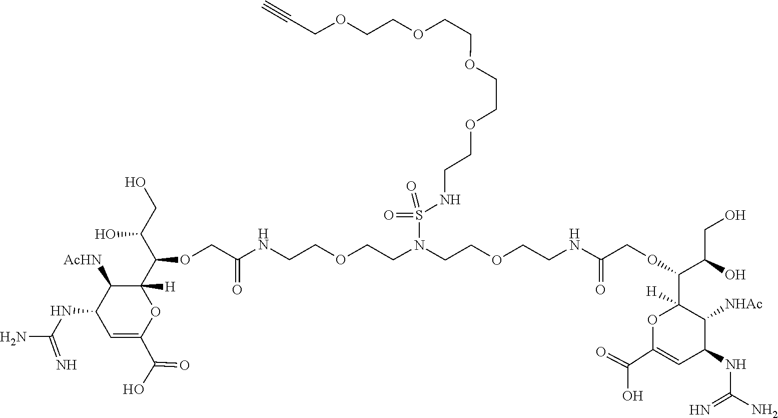 [SMILES: C#CCOCCOCCOCCOCCNS(=O)(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C-Inter-24 | 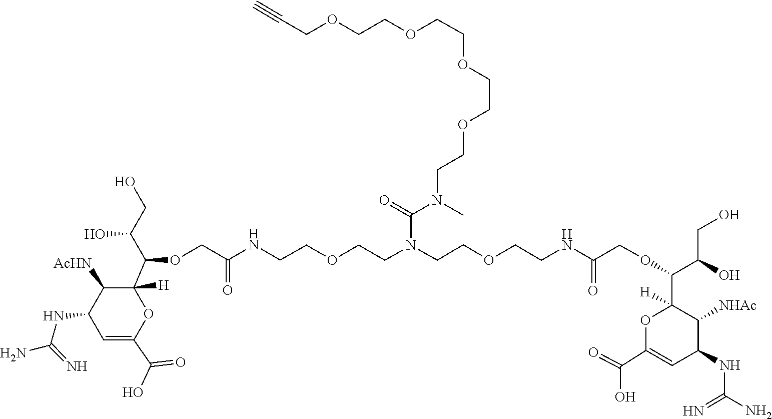 [SMILES: C#CCOCCOCCOCCOCCN(C)C(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C-Inter-25 | 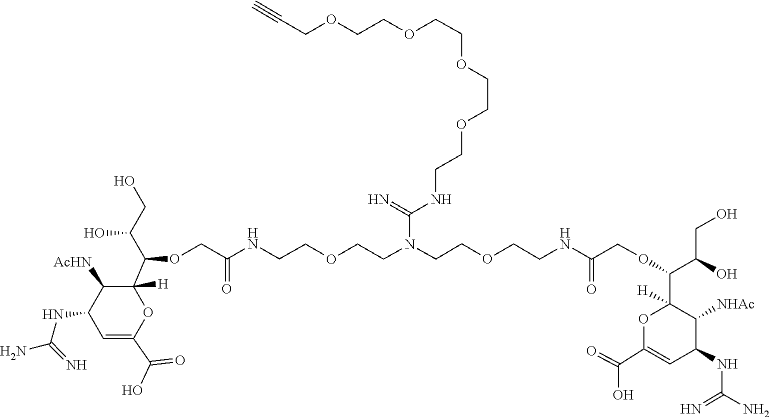 [SMILES: C#CCOCCOCCOCCOCCNC(=N)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C-Inter-26 | 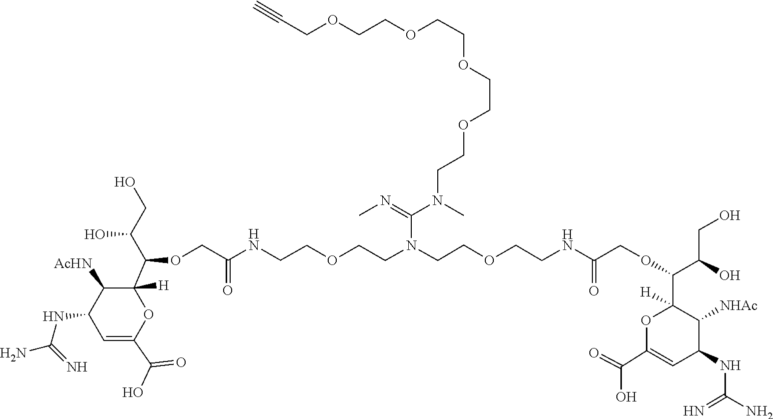 [SMILES: C#CCOCCOCCOCCOCCN(C)/C(=N/C)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C-Inter-27 | 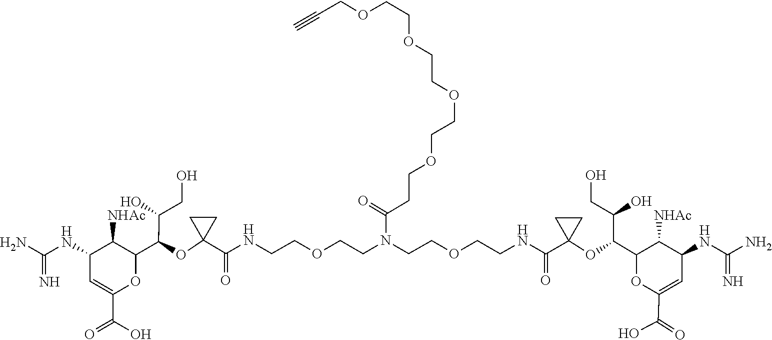 [SMILES: C#CCOCCOCCOCCOCCC(=O)N(CCOCCNC(=O)C1(O[C@@H](C2OC(C(=O)O)=C[C@H](NC(=N)N)[C@H]2NC(C)=O)[C@H](O)CO)CC1)CCOCCNC(=O)C1(O[C@@H](C2OC(C(=O)O)=C[C@H](NC(=N)N)[C@H]2NC(C)=O)[C@H](O)CO)CC1] |
| C-Inter-28 | 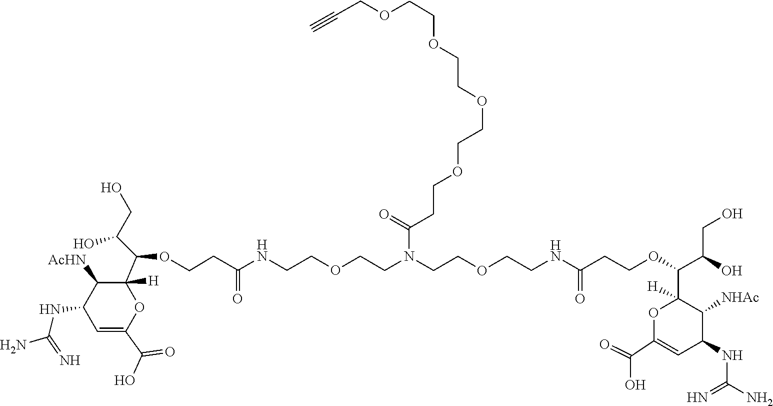 |
| C-Inter-29 | 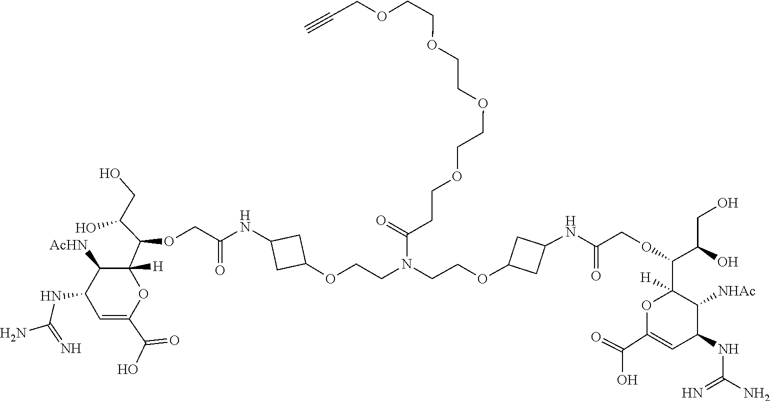 |
| C-Inter-30 | 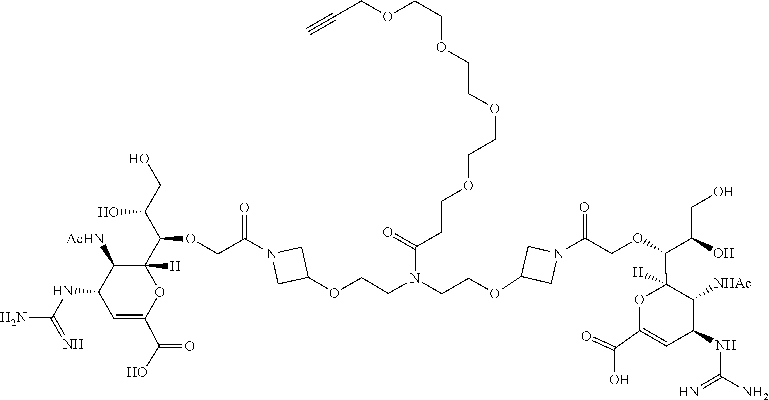 [SMILES: C#CCOCCOCCOCCOCCC(=O)N(CCOC1CN(C(=O)CO[C@@H]([C@@H]2OC(C(=O)O)=C[C@H](NC(=N)N)[C@H]2NC(C)=O)[C@H](O)CO)C1)CCOC1CN(C(=O)CO[C@@H]([C@@H]2OC(C(=O)O)=C[C@H](NC(=N)N)[C@H]2NC(C)=O)[C@H](O)CO)C1] |
| C-Inter-31 | 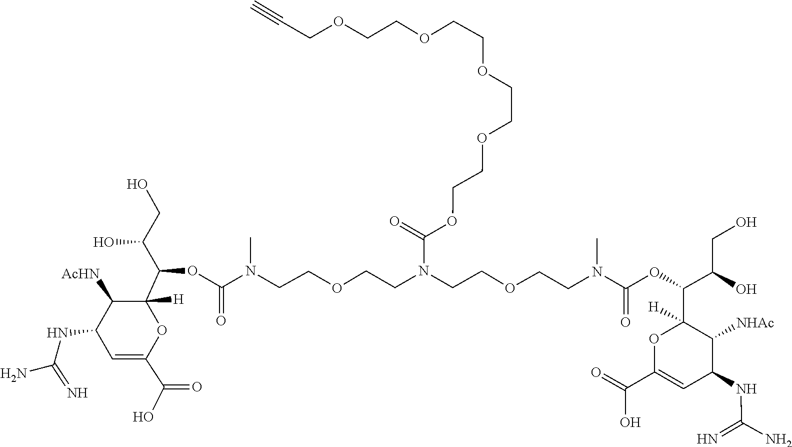 [SMILES: C#CCOCCOCCOCCOCCOC(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-32 | 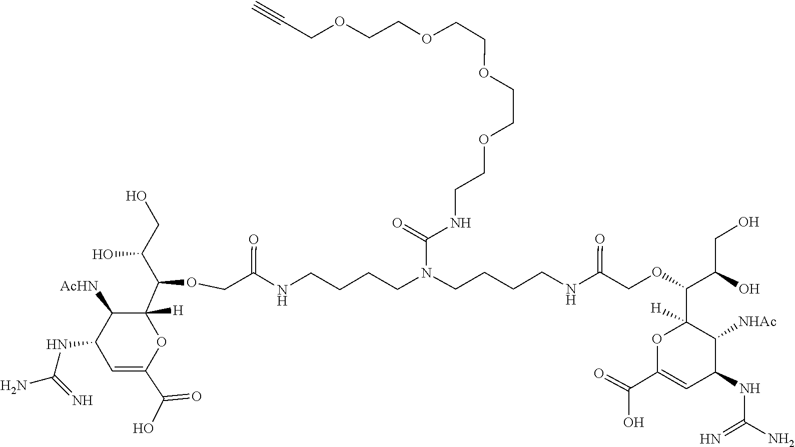 [SMILES: C#CCOCCOCCOCCOCCNC(=O)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| C-Inter-33 | 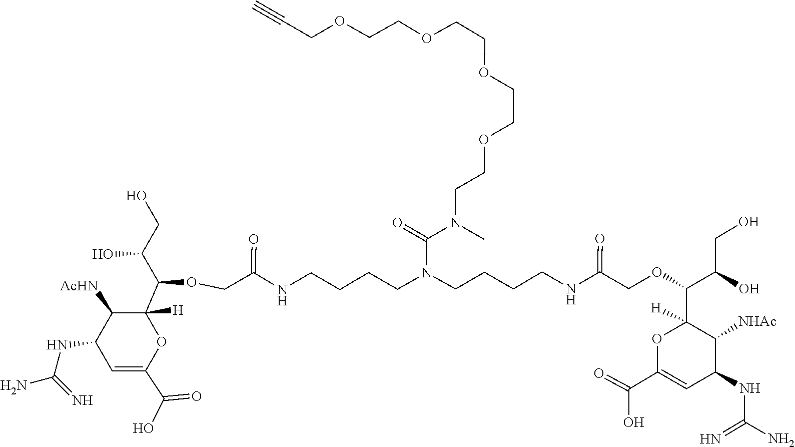 [SMILES: C#CCOCCOCCOCCOCCN(C)C(=O)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| C-Inter-34 | 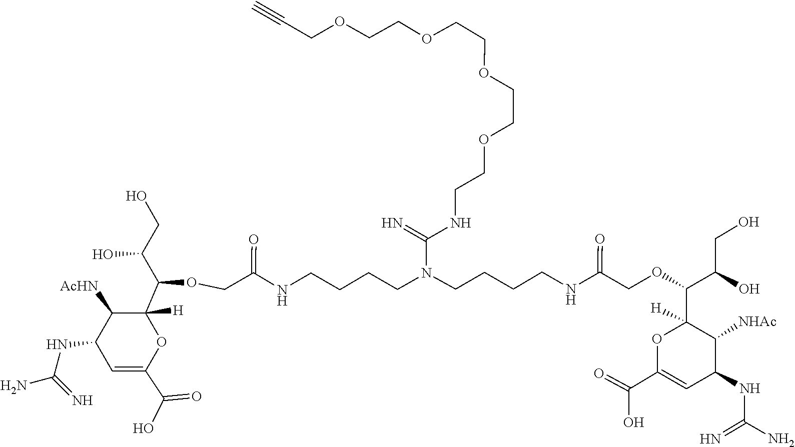 [SMILES: C#CCOCCOCCOCCOCCNC(=N)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| C-Inter-35 | 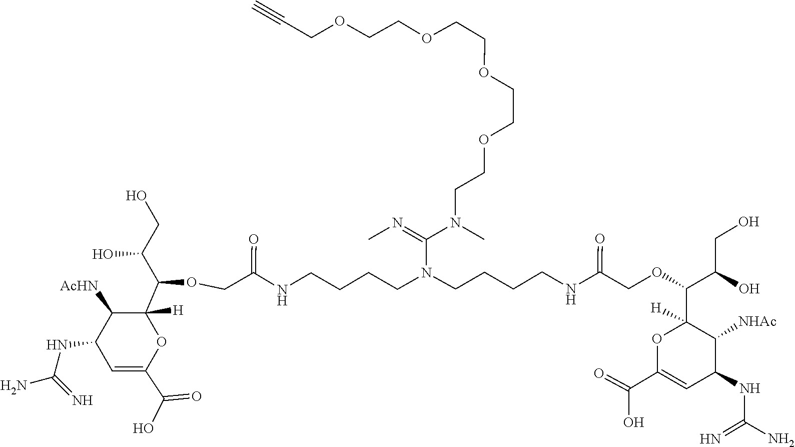 [SMILES: C#CCOCCOCCOCCOCCN(C)/C(=N/C)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| C-Inter-36 | 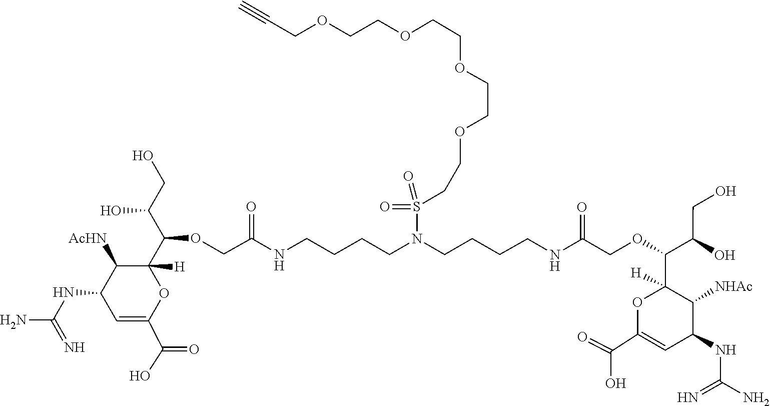 [SMILES: C#CCOCCOCCOCCOCCS(=O)(=O)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| C-Inter-37 | 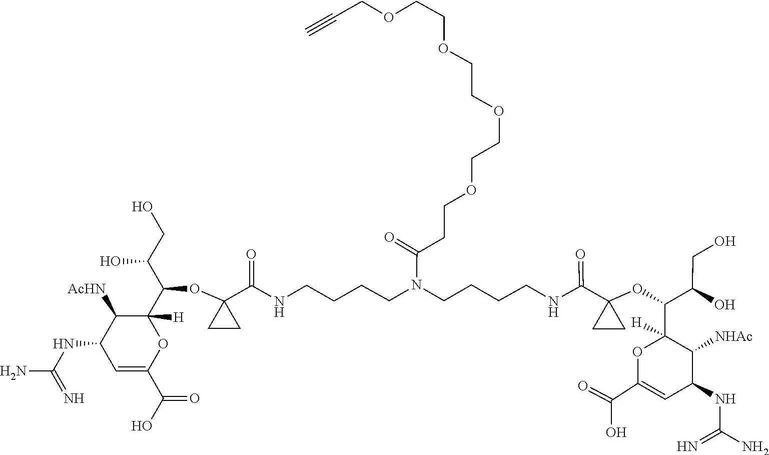 [SMILES: C#CCOCCOCCOCCOCCC(=O)N(CCCCNC(=O)C1(O[C@@H]([C@@H]2OC(C(=O)O)=C[C@H](NC(=N)N)[C@H]2NC(C)=O)[C@H](O)CO)CC1)CCCCNC(=O)C1(O[C@@H]([C@@H]2OC(C(=O)O)=C[C@H](NC(=N)N)[C@H]2NC(C)=O)[C@H](O)CO)CC1] |
| C-Inter-38 | 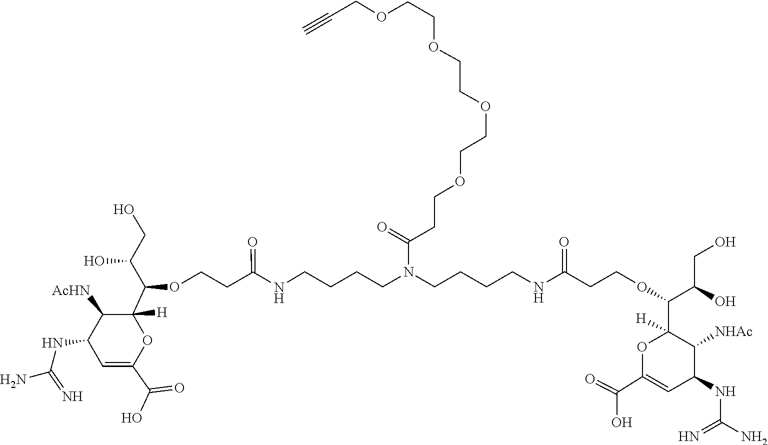 [SMILES: C#CCOCCOCCOCCOCCC(=O)N(CCCCNC(=O)CCO[C@@H]([C@@H]1OC(C(=O)O)=C[C@H](NC(=N)N)[C@H]1NC(C)=O)[C@H](O)CO)CCCCNC(=O)CCO[C@@H]([C@@H]1OC(C(=O)O)=C[C@H](NC(=N)N)[C@H]1NC(C)=O)[C@H](O)CO] |
| C-Inter-39 | 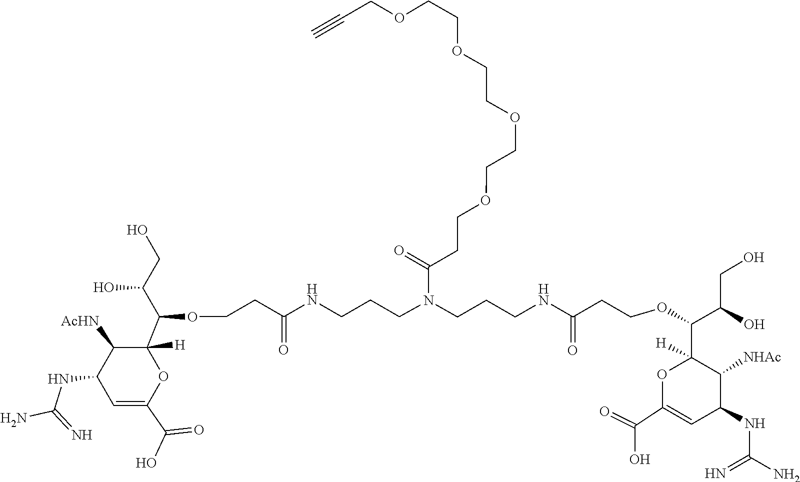 [SMILES: C#CCOCCOCCOCCOCCC(=O)N(CCCNC(=O)CCO[C@@H]([C@@H]1OC(C(=O)O)=C[C@H](NC(=N)N)[C@H]1NC(C)=O)[C@H](O)CO)CCCNC(=O)CCO[C@@H]([C@@H]1OC(C(=O)O)=C[C@H](NC(=N)N)[C@H]1NC(C)=O)[C@H](O)CO] |
| C-Inter-40 | 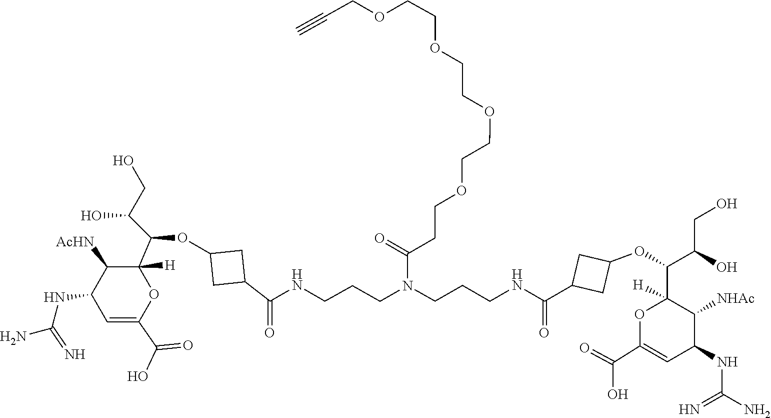 |
| C-Inter-41 | 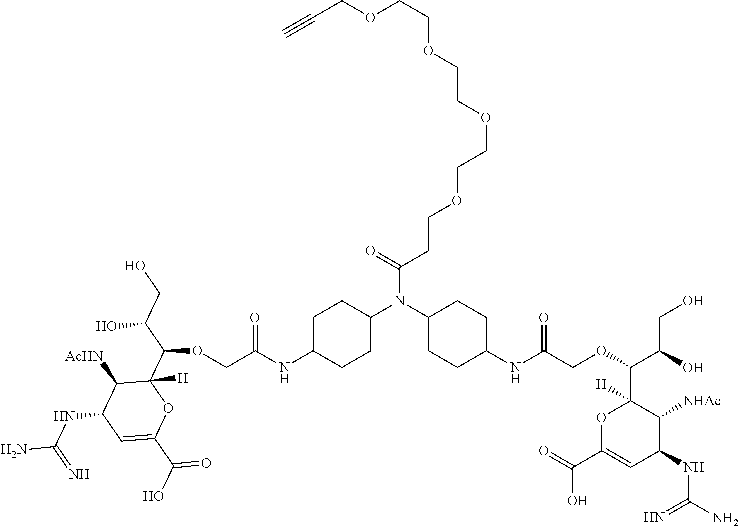 [SMILES: C#CCOCCOCCOCCOCCC(=O)N(C1CCC(NC(=O)CO[C@@H]([C@@H]2OC(C(=O)O)=C[C@H](NC(=N)N)[C@H]2NC(C)=O)[C@H](O)CO)CC1)C1CCC(NC(=O)CO[C@@H]([C@@H]2OC(C(=O)O)=C[C@H](NC(=N)N)[C@H]2NC(C)=O)[C@H](O)CO)CC1] |
| C-Inter-42 | 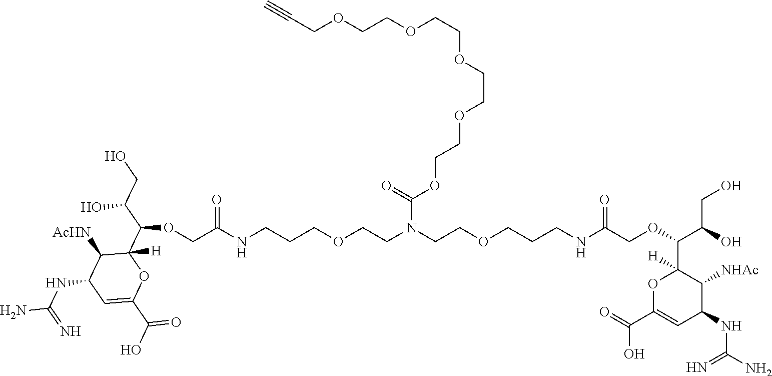 [SMILES: C#CCOCCOCCOCCOCCOC(=O)N(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| C-Inter-43 | 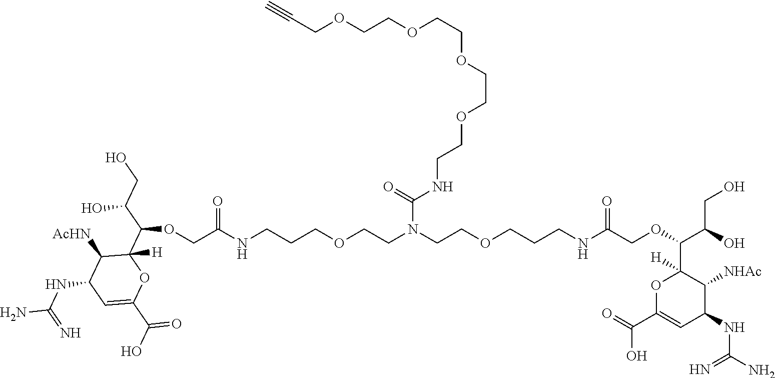 [SMILES: C#CCOCCOCCOCCOCCNC(=O)N(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| C-Inter-44 | 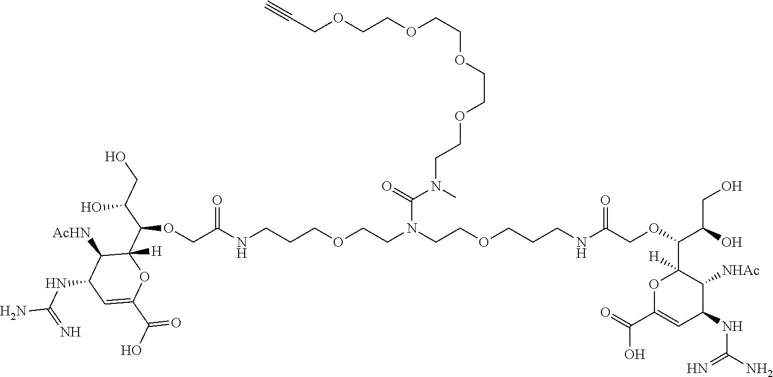 [SMILES: C#CCOCCOCCOCCOCCN(C)C(=O)N(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| C-Inter-45 | 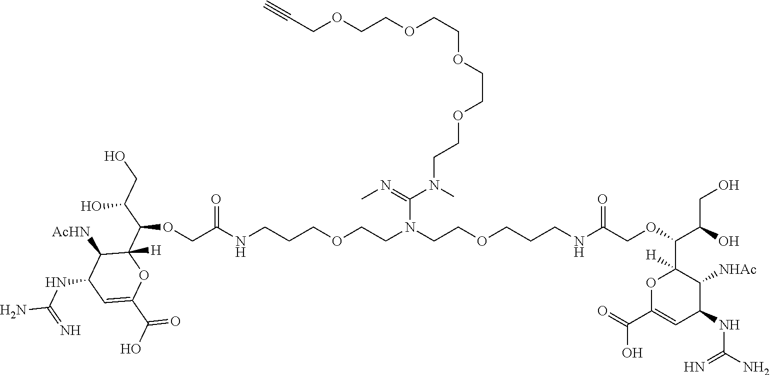 [SMILES: C#CCOCCOCCOCCOCCN(C)/C(=N/C)N(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| C-Inter-46 | 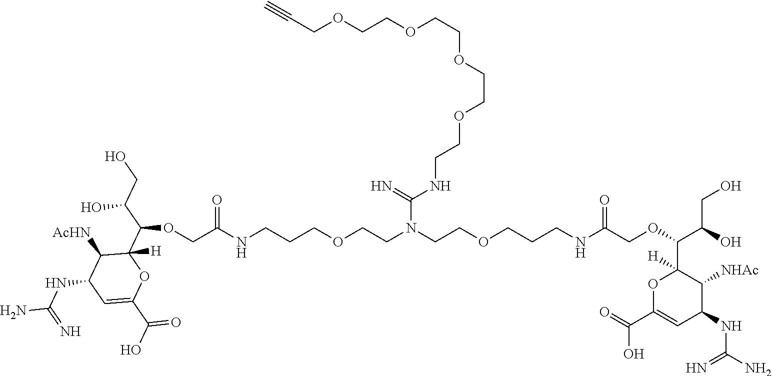 [SMILES: C#CCOCCOCCOCCOCCNC(=N)N(CCOCCCNC(=O)CO[C@@H]([C@@H]1OC(C(=O)O)=C[C@H](NC(=N)N)[C@H]1NC(C)=O)[C@H](O)CO)CCOCCCNC(=O)CO[C@@H]([C@@H]1OC(C(=O)O)=C[C@H](NC(=N)N)[C@H]1NC(C)=O)[C@H](O)CO] |
| C-Inter-47 | 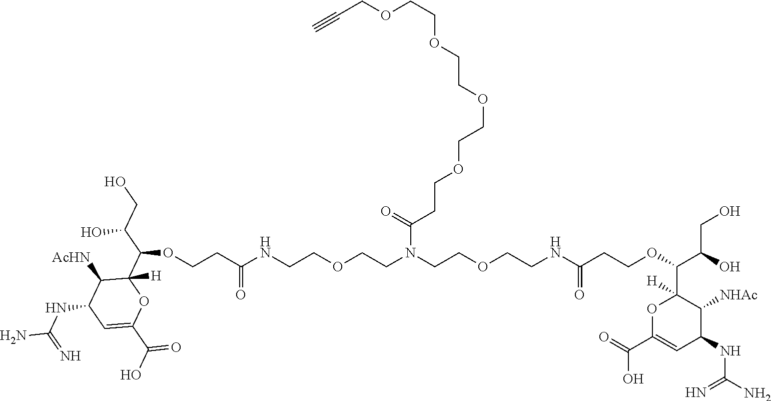 [SMILES: C#CCOCCOCCOCCOCCC(=O)N(CCOCCNC(=O)CCO[C@@H]([C@@H]1OC(C(=O)O)=C[C@H](NC(=N)N)[C@H]1NC(C)=O)[C@H](O)CO)CCOCCNC(=O)CCO[C@@H]([C@@H]1OC(C(=O)O)=C[C@H](NC(=N)N)[C@H]1NC(C)=O)[C@H](O)CO] |
| C-Inter-48 | 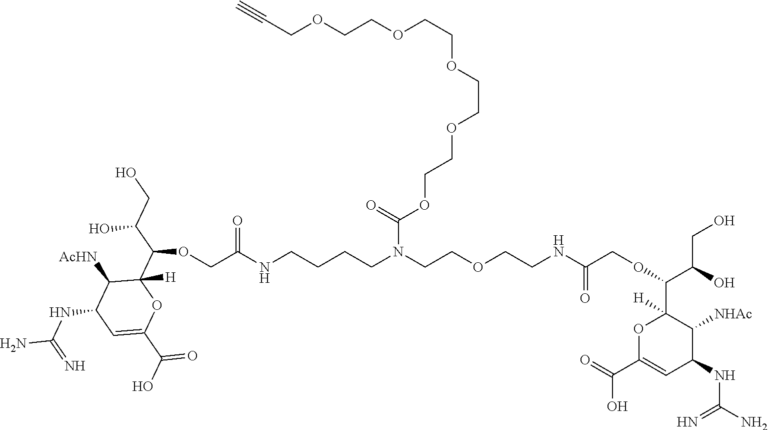 [SMILES: C#CCOCCOCCOCCOCCOC(=O)N(CCCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| C-Inter-49 | 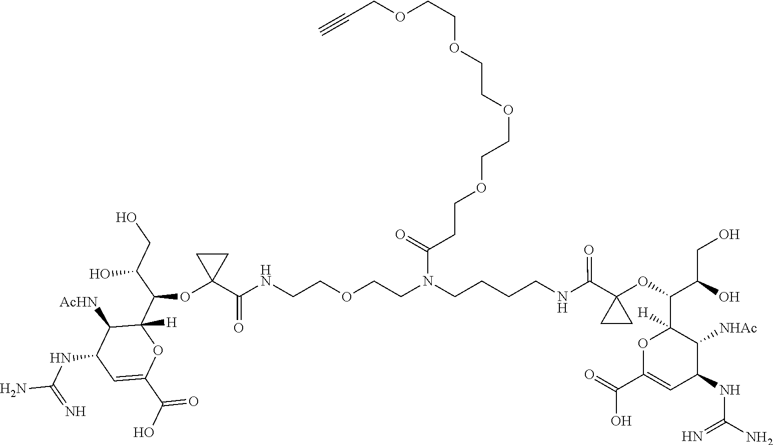 [SMILES: C#CCOCCOCCOCCOCCC(=O)N(CCCCNC(=O)C1(O[C@@H]([C@@H]2OC(C(=O)O)=C[C@H](NC(=N)N)[C@H]2NC(C)=O)[C@H](O)CO)CC1)CCOCCNC(=O)C1(O[C@@H]([C@@H]2OC(C(=O)O)=C[C@H](NC(=N)N)[C@H]2NC(C)=O)[C@H](O)CO)CC1] |
| C-Inter-50 | 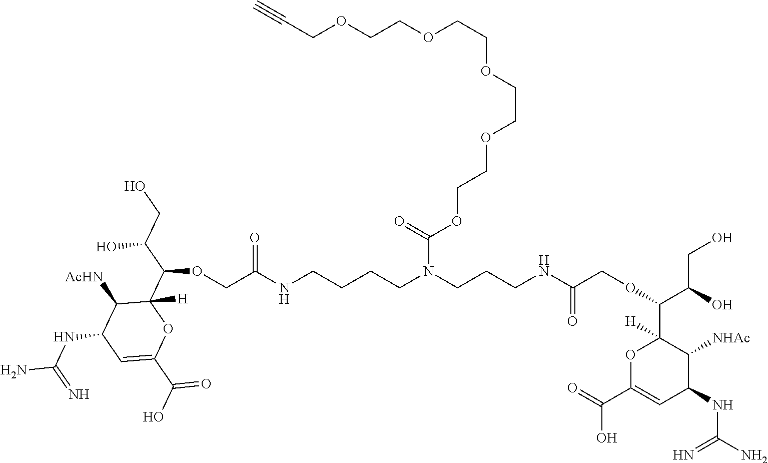 [SMILES: C#CCOCCOCCOCCOCCOC(=O)N(CCCCNC(=O)CO[C@@H]([C@@H]1OC(C(=O)O)=C[C@H](NC(=N)N)[C@H]1NC(C)=O)[C@H](O)CO)CCCNC(=O)CO[C@@H]([C@@H]1OC(C(=O)O)=C[C@H](NC(=N)N)[C@H]1NC(C)=O)[C@H](O)CO] |
| C-Inter-51 | 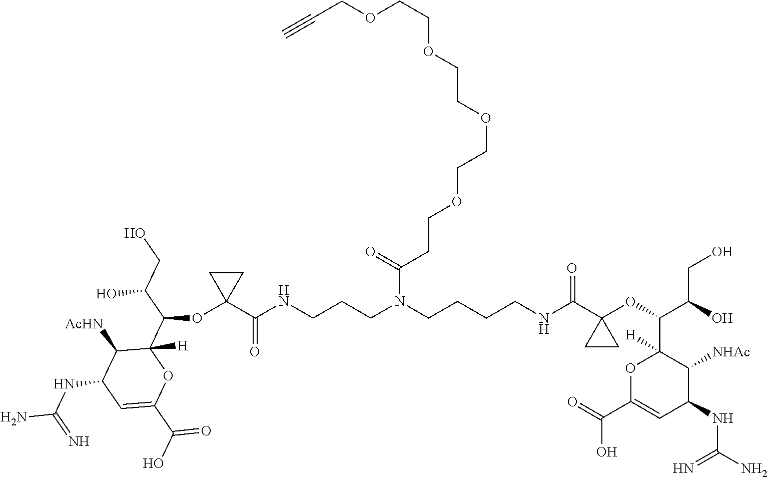 [SMILES: C#CCOCCOCCOCCOCCC(=O)N(CCCCNC(=O)C1(O[C@@H]([C@@H]2OC(C(=O)O)=C[C@H](NC(=N)N)[C@H]2NC(C)=O)[C@H](O)CO)CC1)CCCNC(=O)C1(O[C@@H]([C@@H]2OC(C(=O)O)=C[C@H](NC(=N)N)[C@H]2NC(C)=O)[C@H](O)CO)CC1] |
| C-Inter-52 | 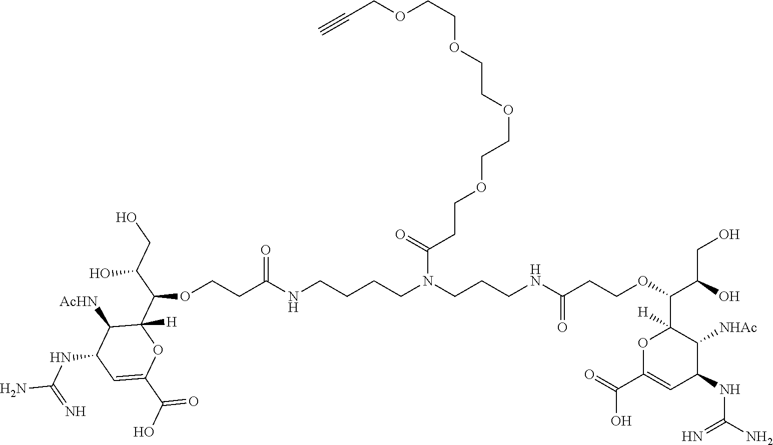 [SMILES: C#CCOCCOCCOCCOCCC(=O)N(CCCCNC(=O)CCO[C@@H]([C@@H]1OC(C(=O)O)=C[C@H](NC(=N)N)[C@H]1NC(C)=O)[C@H](O)CO)CCCNC(=O)CCO[C@@H]([C@@H]1OC(C(=O)O)=C[C@H](NC(=N)N)[C@H]1NC(C)=O)[C@H](O)CO] |
| C-Inter-53 | 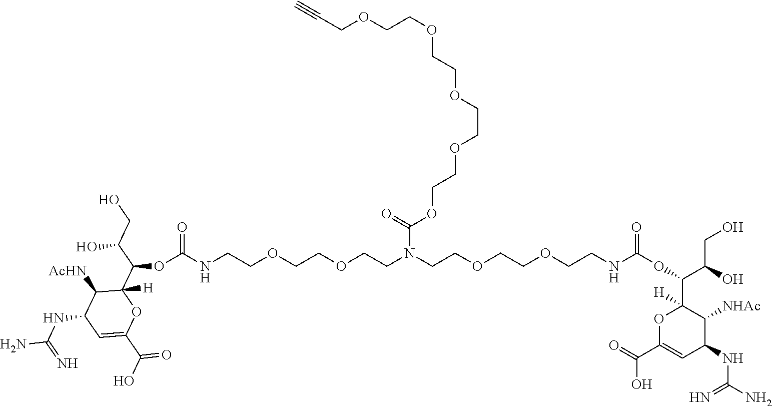 [SMILES: C#CCOCCOCCOCCOCCOC(=O)N(CCOCCOCCNC(=O)O[C@@H]([C@@H]1OC(C(=O)O)=C[C@H](NC(=N)N)[C@H]1NC(C)=O)[C@H](O)CO)CCOCCOCCNC(=O)O[C@@H]([C@@H]1OC(C(=O)O)=C[C@H](NC(=N)N)[C@H]1NC(C)=O)[C@H](O)CO] |
| C-Inter-54 | 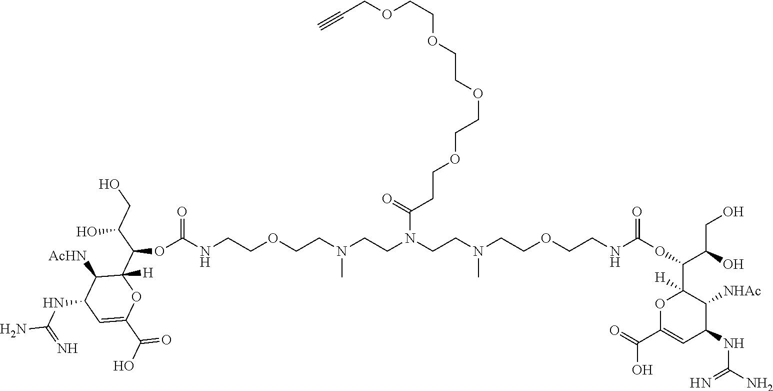 [SMILES: C#CCOCCOCCOCCOCCC(=O)N(CCN(C)CCOCCNC(=O)O[C@@H]([C@@H]1OC(C(=O)O)=C[C@H](NC(=N)N)[C@H]1NC(C)=O)[C@H](O)CO)CCN(C)CCOCCNC(=O)O[C@@H]([C@@H]1OC(C(=O)O)=C[C@H](NC(=N)N)[C@H]1NC(C)=O)[C@H](O)CO] |
| C-Inter-55 | 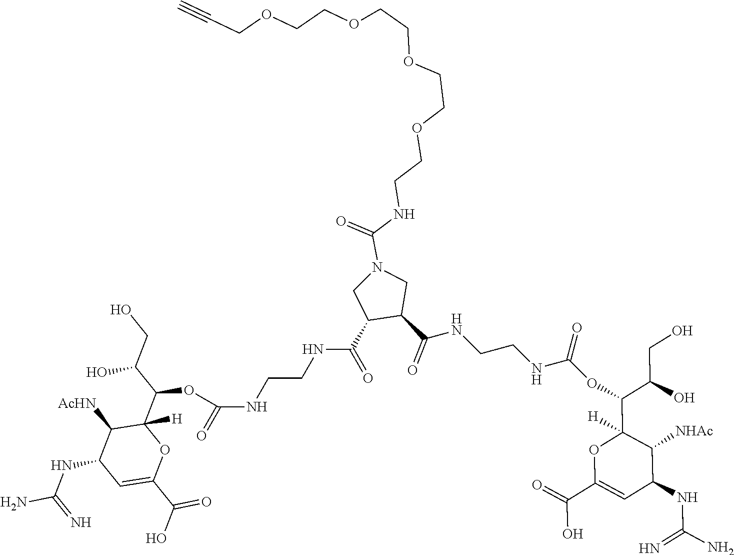 [SMILES: C#CCOCCOCCOCCOCCNC(=O)N1C[C@@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C-Inter-56 | 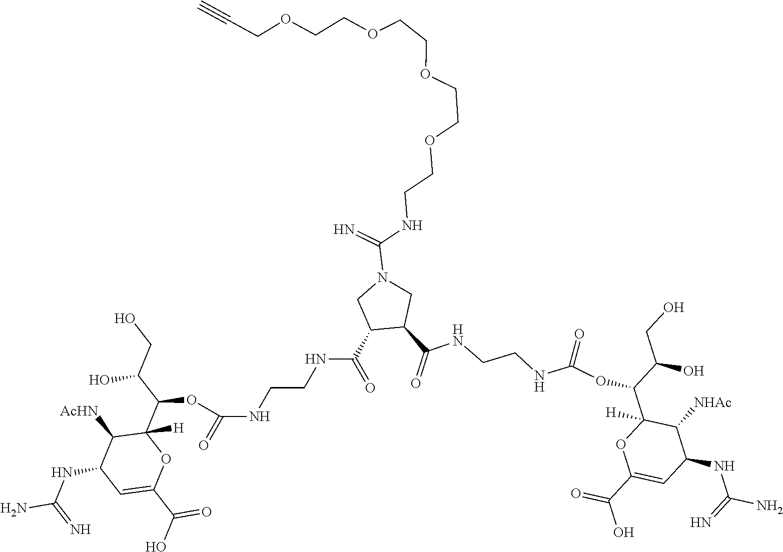 [SMILES: C#CCOCCOCCOCCOCCNC(=N)N1C[C@@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C-Inter-57 | 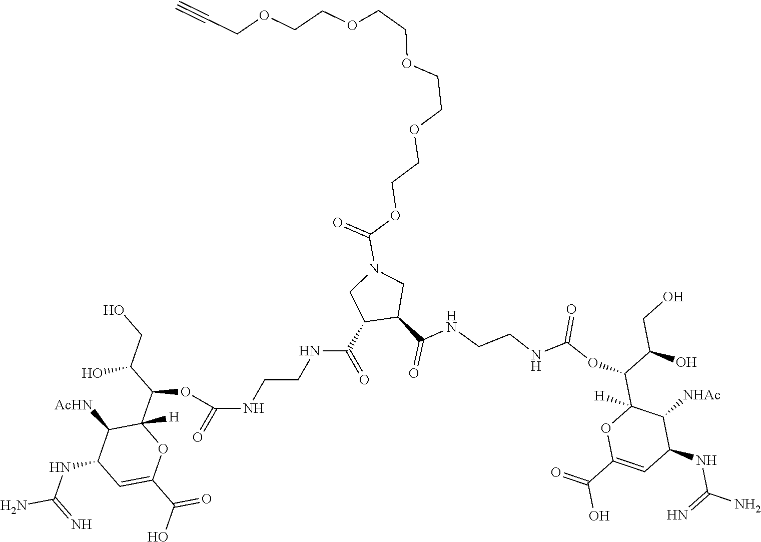 [SMILES: C#CCOCCOCCOCCOCCOC(=O)N1C[C@@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C-Inter-58 | 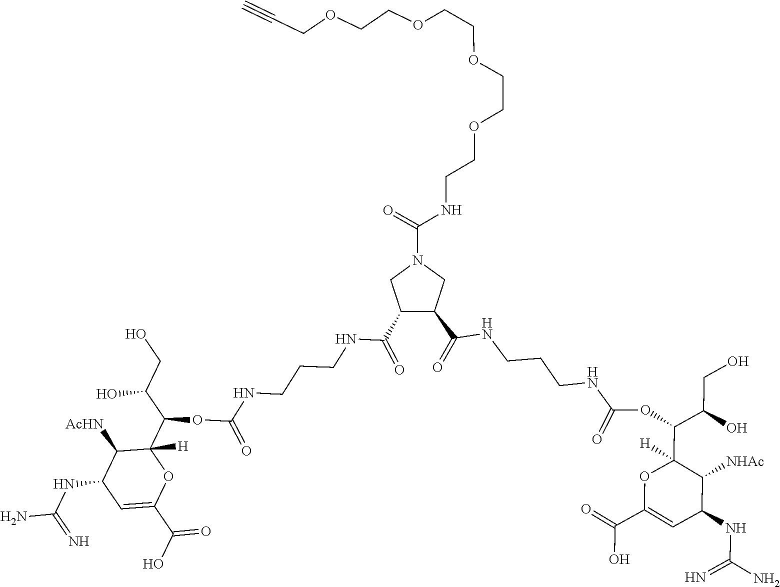 [SMILES: C#CCOCCOCCOCCOCCNC(=O)N1C[C@@H](C(=O)NCCCNC(=O)O[C@@H]([C@@H]2OC(C(=O)O)=C[C@H](NC(=N)N)[C@H]2NC(C)=O)[C@H](O)CO)[C@H](C(=O)NCCCNC(=O)O[C@@H]([C@@H]2OC(C(=O)O)=C[C@H](NC(=N)N)[C@H]2NC(C)=O)[C@H](O)CO)C1] |
| C-Inter-59 | 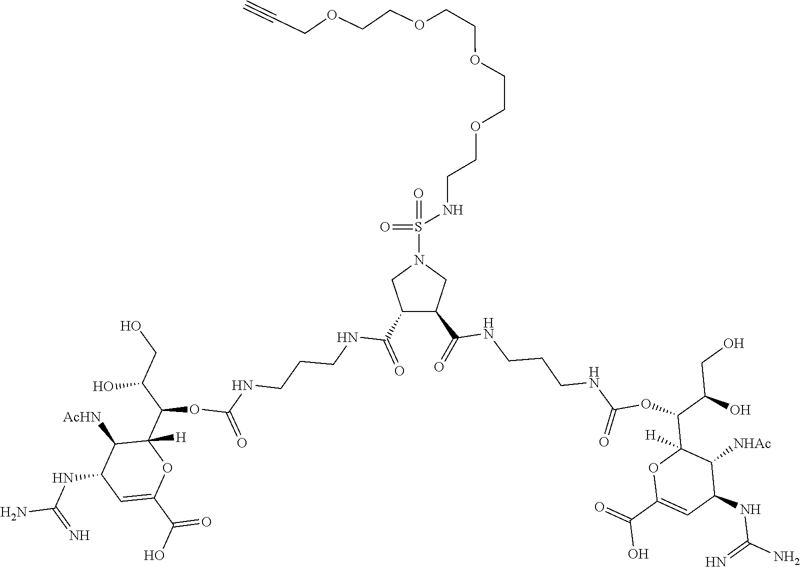 [SMILES: C#CCOCCOCCOCCOCCNS(=O)(=O)N1C[C@@H](C(=O)NCCCNC(=O)O[C@@H]([C@@H]2OC(C(=O)O)=C[C@H](NC(=N)N)[C@H]2NC(C)=O)[C@H](O)CO)[C@H](C(=O)NCCCNC(=O)O[C@@H]([C@@H]2OC(C(=O)O)=C[C@H](NC(=N)N)[C@H]2NC(C)=O)[C@H](O)CO)C1] |
| C-Inter-60 | 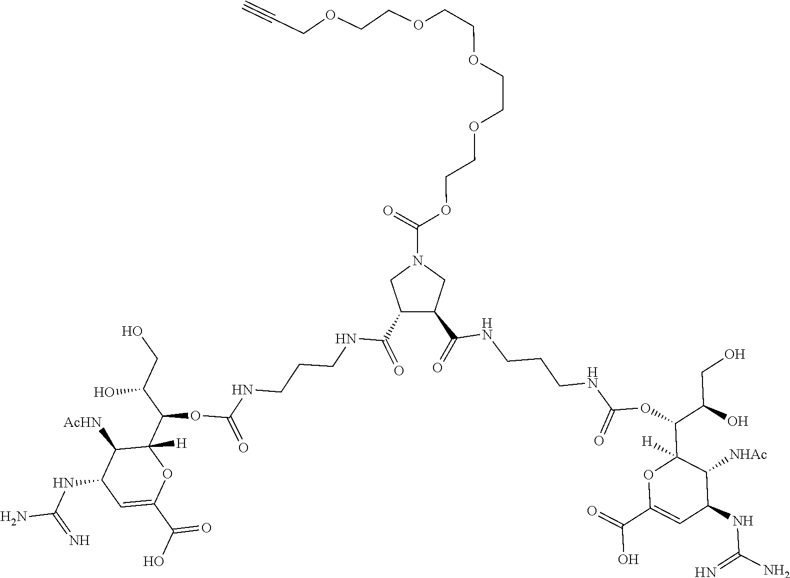 [SMILES: C#CCOCCOCCOCCOCCOC(=O)N1C[C@@H](C(=O)NCCCNC(=O)O[C@@H]([C@@H]2OC(C(=O)O)=C[C@H](NC(=N)N)[C@H]2NC(C)=O)[C@H](O)CO)[C@H](C(=O)NCCCNC(=O)O[C@@H]([C@@H]2OC(C(=O)O)=C[C@H](NC(=N)N)[C@H]2NC(C)=O)[C@H](O)CO)C1] |
| C-Inter-61 | 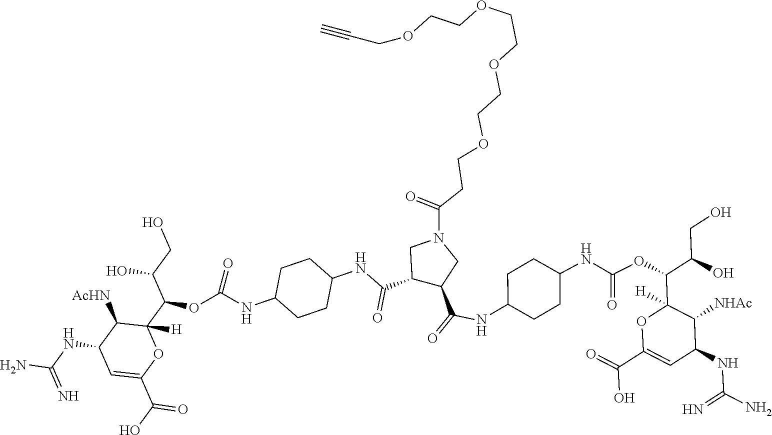 [SMILES: C#CCOCCOCCOCCOCCC(=O)N1C[C@@H](C(=O)NC2CCC(NC(=O)O[C@@H]([C@@H]3OC(C(=O)O)=C[C@H](NC(=N)N)[C@H]3NC(C)=O)[C@H](O)CO)CC2)[C@H](C(=O)NC2CCC(NC(=O)O[C@@H]([C@@H]3OC(C(=O)O)=C[C@H](NC(=N)N)[C@H]3NC(C)=O)[C@H](O)CO)CC2)C1] |
| C-Inter-62 | 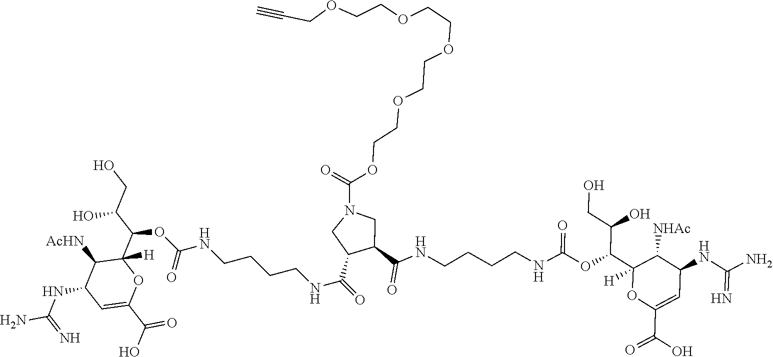 [SMILES: C#CCOCCOCCOCCOCCOC(=O)N1C[C@@H](C(=O)NCCCCNC(=O)O[C@@H]([C@@H]2OC(C(=O)O)=C[C@H](NC(=N)N)[C@H]2NC(C)=O)[C@H](O)CO)[C@H](C(=O)NCCCCNC(=O)O[C@@H]([C@@H]2OC(C(=O)O)=C[C@H](NC(=N)N)[C@H]2NC(C)=O)[C@H](O)CO)C1] |
| C-Inter-63 | 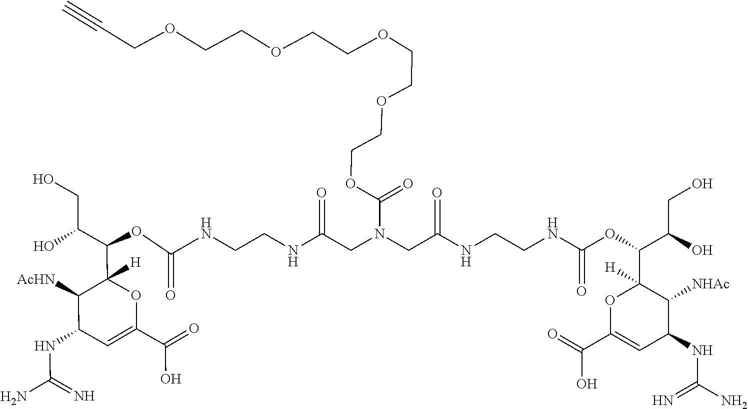 [SMILES: C#CCOCCOCCOCCOCCOC(=O)N(CC(=O)NCCNC(=O)O[C@@H]([C@@H]1OC(C(=O)O)=C[C@H](NC(=N)N)[C@H]1NC(C)=O)[C@H](O)CO)CC(=O)NCCNC(=O)O[C@@H]([C@@H]1OC(C(=O)O)=C[C@H](NC(=N)N)[C@H]1NC(C)=O)[C@H](O)CO] |
| C-Inter-64 | 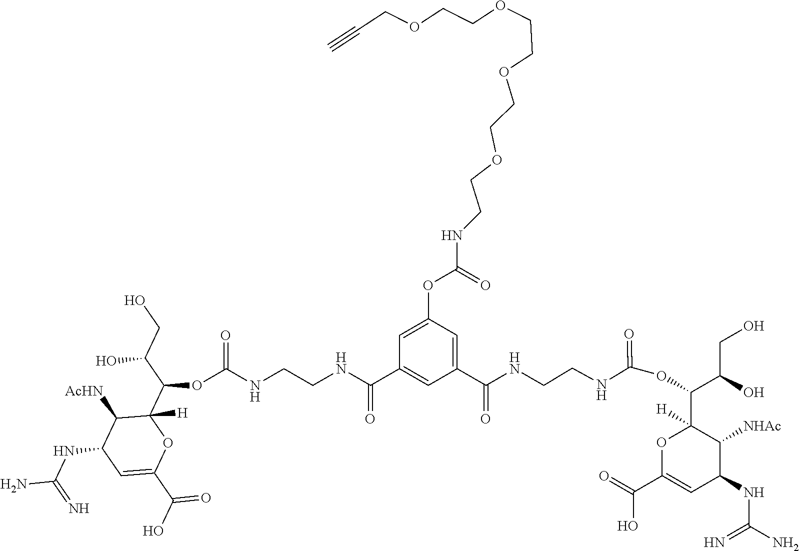 [SMILES: C#CCOCCOCCOCCOCCNC(=O)Oc1cc(C(=O)NCCNC(=O)O[C@@H]([C@@H]2OC(C(=O)O)=C[C@H](NC(=N)N)[C@H]2NC(C)=O)[C@H](O)CO)cc(C(=O)NCCNC(=O)O[C@@H]([C@@H]2OC(C(=O)O)=C[C@H](NC(=N)N)[C@H]2NC(C)=O)[C@H](O)CO)c1] |
| C-Inter-65 | 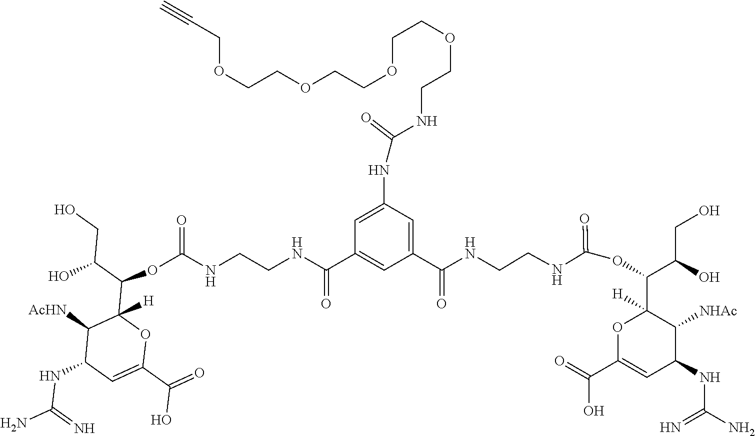 [SMILES: C#CCOCCOCCOCCOCCNC(=O)Nc1cc(C(=O)NCCNC(=O)O[C@@H]([C@@H]2OC(C(=O)O)=C[C@H](NC(=N)N)[C@H]2NC(C)=O)[C@H](O)CO)cc(C(=O)NCCNC(=O)O[C@@H]([C@@H]2OC(C(=O)O)=C[C@H](NC(=N)N)[C@H]2NC(C)=O)[C@H](O)CO)c1] |
| C-Inter-66 | 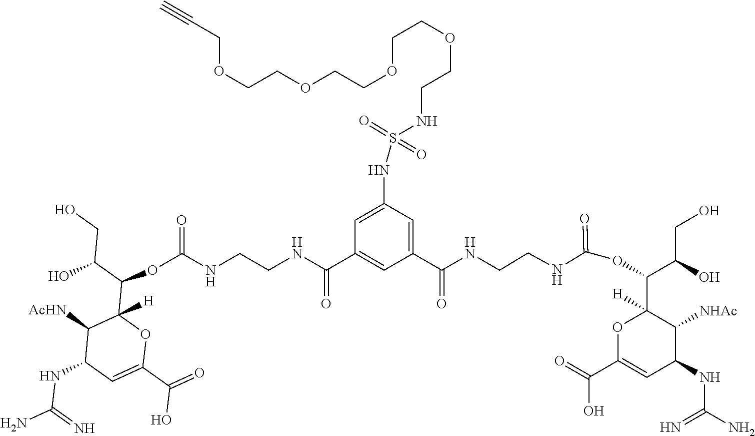 [SMILES: C#CCOCCOCCOCCOCCNS(=O)(=O)Nc1cc(C(=O)NCCNC(=O)O[C@@H]([C@@H]2OC(C(=O)O)=C[C@H](NC(=N)N)[C@H]2NC(C)=O)[C@H](O)CO)cc(C(=O)NCCNC(=O)O[C@@H]([C@@H]2OC(C(=O)O)=C[C@H](NC(=N)N)[C@H]2NC(C)=O)[C@H](O)CO)c1] |
| C-Inter-67 | 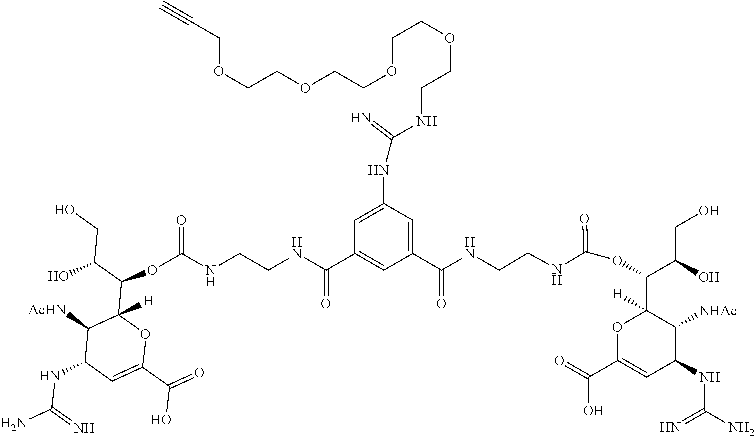 [SMILES: C#CCOCCOCCOCCOCCNC(=N)Nc1cc(C(=O)NCCNC(=O)O[C@@H]([C@@H]2OC(C(=O)O)=C[C@H](NC(=N)N)[C@H]2NC(C)=O)[C@H](O)CO)cc(C(=O)NCCNC(=O)O[C@@H]([C@@H]2OC(C(=O)O)=C[C@H](NC(=N)N)[C@H]2NC(C)=O)[C@H](O)CO)c1] |
| C-Inter-68 | 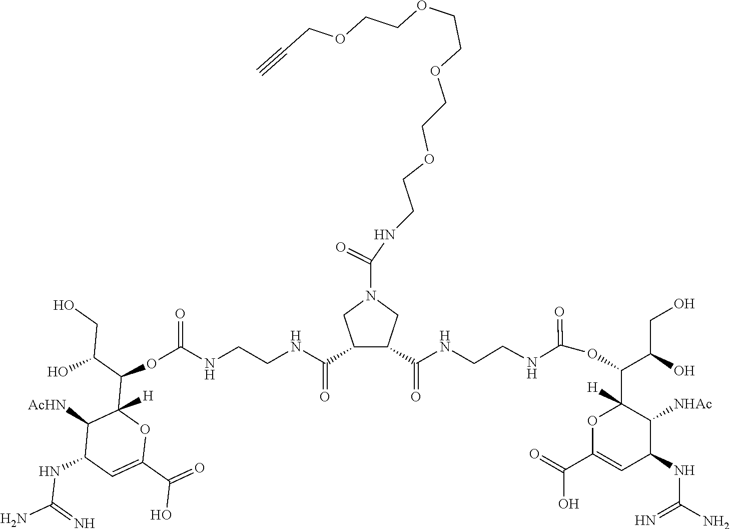 [SMILES: C#CCOCCOCCOCCOCCNC(=O)N1C[C@H](C(=O)NCCNC(=O)O[C@@H]([C@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C-Inter-69 | 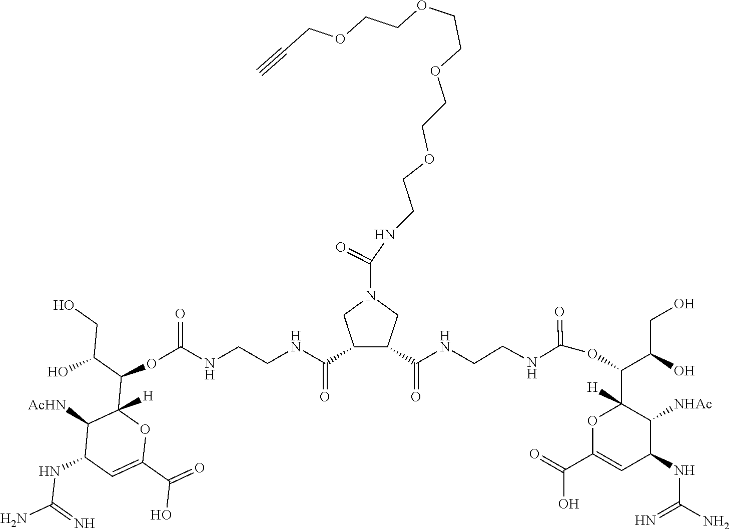 [SMILES: C#CCOCCOCCOCCOCCNC(=O)N1C[C@H](C(=O)NCCNC(=O)O[C@@H]([C@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C-Inter-70 | 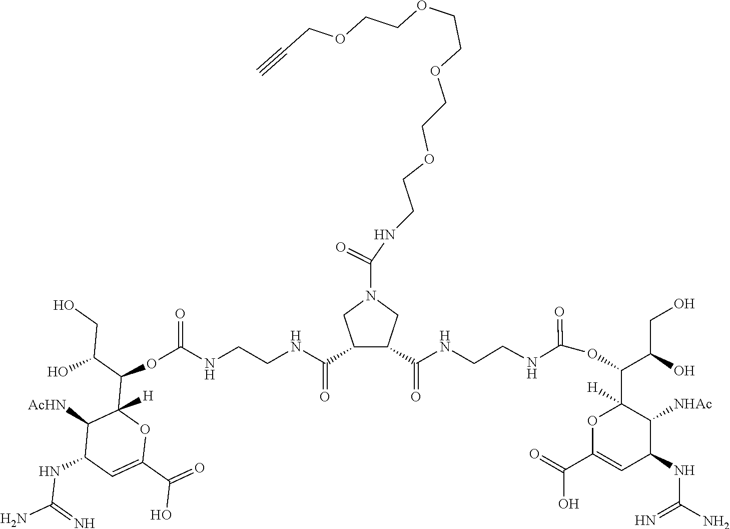 [SMILES: C#CCOCCOCCOCCOCCNC(=O)N1C[C@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C-Inter-71 | 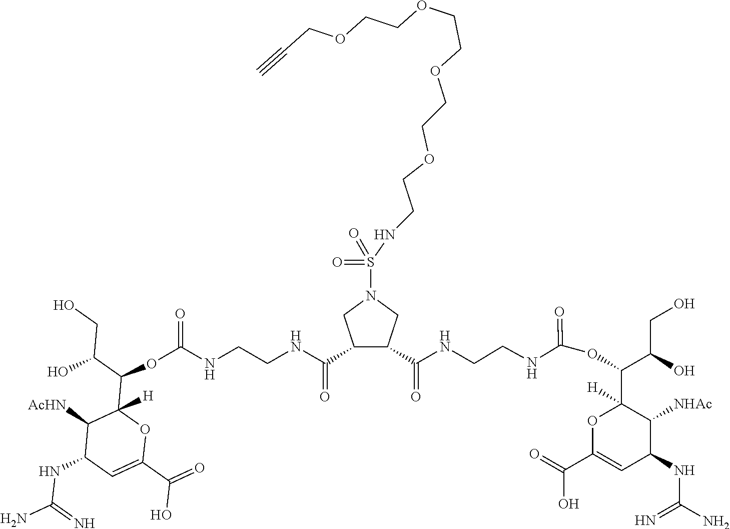 [SMILES: C#CCOCCOCCOCCOCCNS(=O)(=O)N1C[C@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| C-Inter-72 | 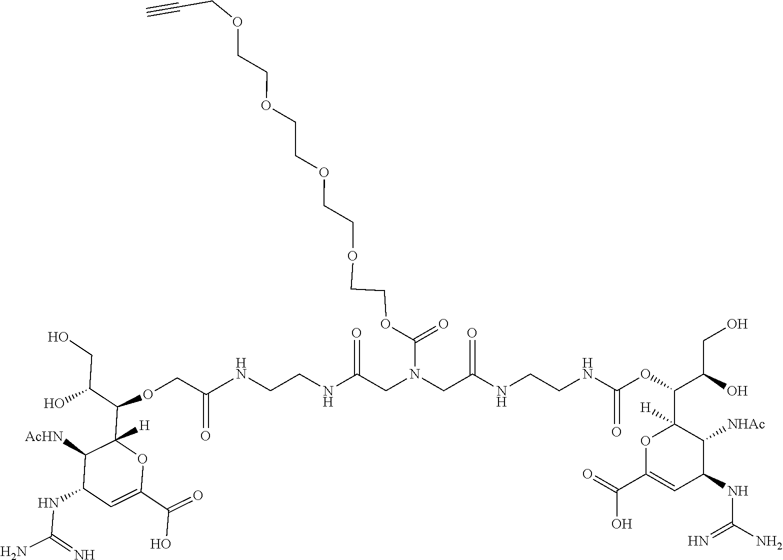 [SMILES: C#CCOCCOCCOCCOCCOC(=O)N(CC(=O)NCCNC(=O)CO[C@@H]([C@@H]1OC(C(=O)O)=C[C@H](NC(=N)N)[C@H]1NC(C)=O)[C@H](O)CO)CC(=O)NCCNC(=O)O[C@@H]([C@@H]1OC(C(=O)O)=C[C@H](NC(=N)N)[C@H]1NC(C)=O)[C@H](O)CO] |
| C-Inter-73 | 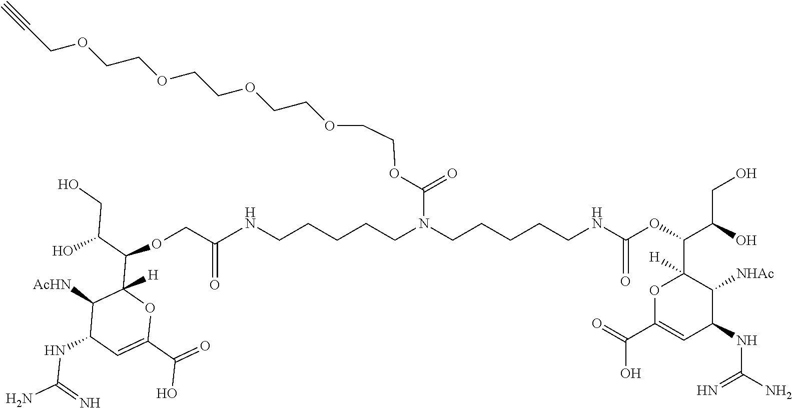 [SMILES: C#CCOCCOCCOCCOCCOC(=O)N(CCCCCNC(=O)CO[C@@H]([C@@H]1OC(C(=O)O)=C[C@H](NC(=N)N)[C@H]1NC(C)=O)[C@H](O)CO)CCCCCNC(=O)O[C@@H]([C@@H]1OC(C(=O)O)=C[C@H](NC(=N)N)[C@H]1NC(C)=O)[C@H](O)CO] |
| C-Inter-74 | 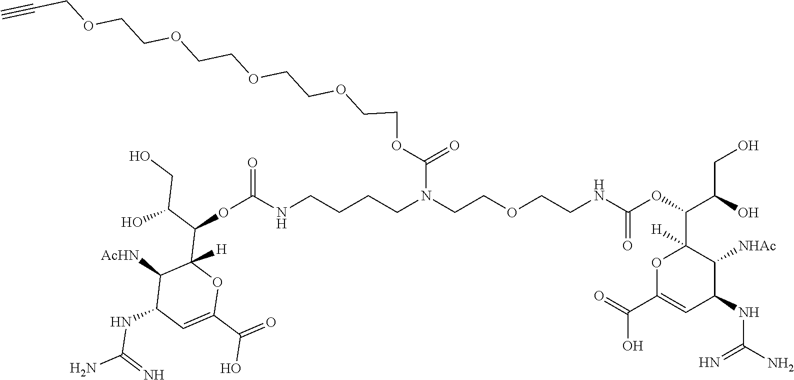 [SMILES: C#CCOCCOCCOCCOCCOC(=O)N(CCCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| C-Inter-75 | 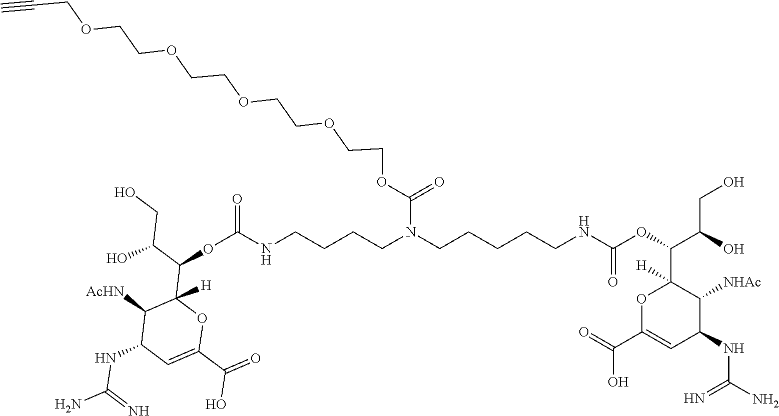 [SMILES: C#CCOCCOCCOCCOCCOC(=O)N(CCCCCNC(=O)O[C@@H]([C@@H]1OC(C(=O)O)=C[C@H](NC(=N)N)[C@H]1NC(C)=O)[C@H](O)CO)CCCCNC(=O)O[C@@H]([C@@H]1OC(C(=O)O)=C[C@H](NC(=N)N)[C@H]1NC(C)=O)[C@H](O)CO] |
| C-Inter-76 | 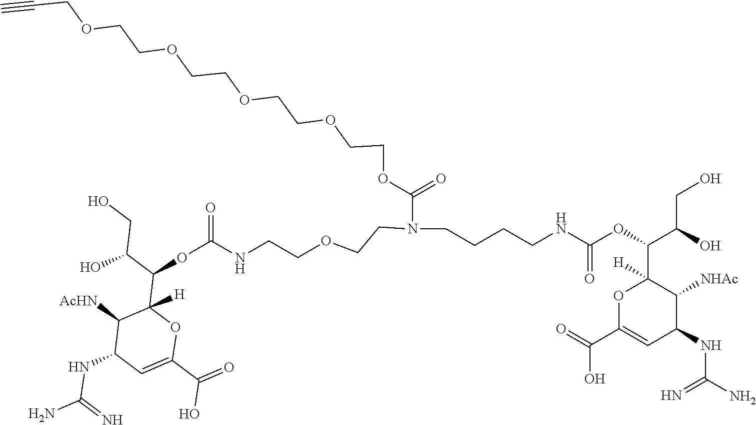 [SMILES: C#CCOCCOCCOCCOCCOC(=O)N(CCCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| C-Inter-77 | 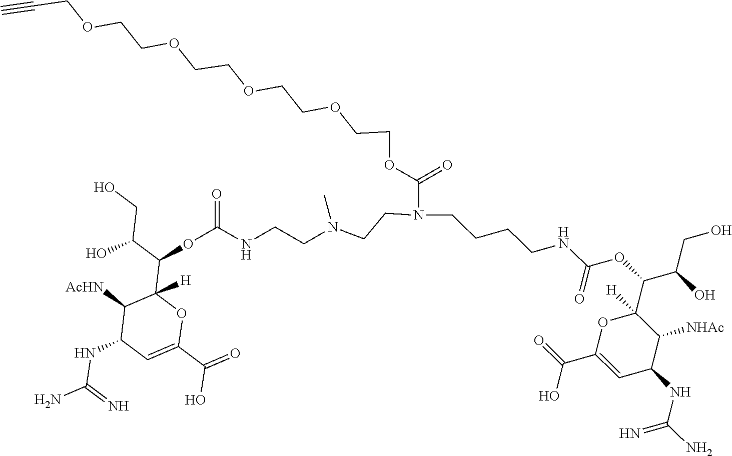 [SMILES: C#CCOCCOCCOCCOCCOC(=O)N(CCCCNC(=O)O[C@@H]([C@@H]1OC(C(=O)O)=C[C@H](NC(=N)N)[C@H]1NC(C)=O)[C@H](O)CO)CCN(C)CCNC(=O)O[C@@H]([C@@H]1OC(C(=O)O)=C[C@H](NC(=N)N)[C@H]1NC(C)=O)[C@H](O)CO] |
| C-Inter-78 | 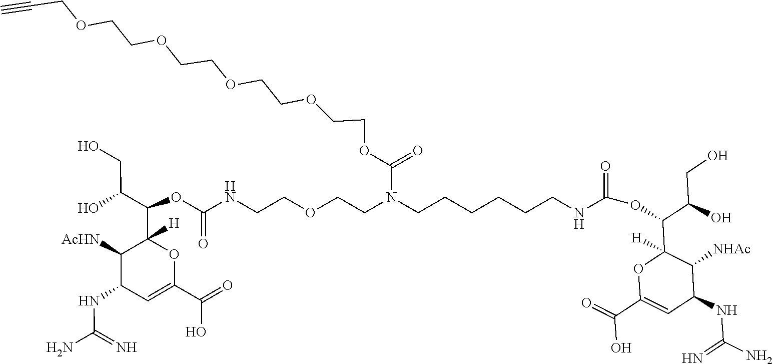 [SMILES: C#CCOCCOCCOCCOCCOC(=O)N(CCCCCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| C-Inter-79 | 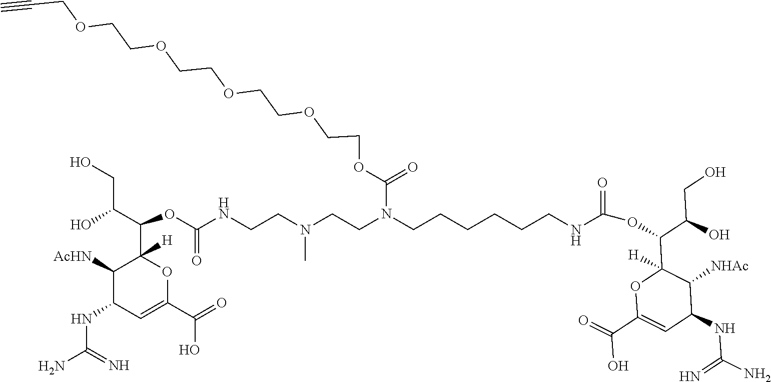 [SMILES: C#CCOCCOCCOCCOCCOC(=O)N(CCCCCCNC(=O)O[C@@H]([C@@H]1OC(C(=O)O)=C[C@H](NC(=N)N)[C@H]1NC(C)=O)[C@H](O)CO)CCN(C)CCNC(=O)O[C@@H]([C@@H]1OC(C(=O)O)=C[C@H](NC(=N)N)[C@H]1NC(C)=O)[C@H](O)CO] |
| C-Inter-80 | 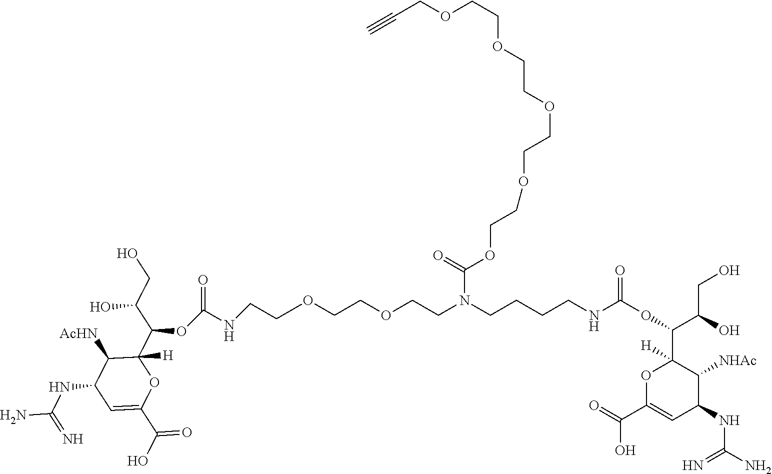 [SMILES: C#CCOCCOCCOCCOCCOC(=O)N(CCCCNC(=O)O[C@@H]([C@@H]1OC(C(=O)O)=C[C@H](NC(=N)N)[C@H]1NC(C)=O)[C@H](O)CO)CCOCCOCCNC(=O)O[C@@H]([C@@H]1OC(C(=O)O)=C[C@H](NC(=N)N)[C@H]1NC(C)=O)[C@H](O)CO] |
| C-Inter-81 | 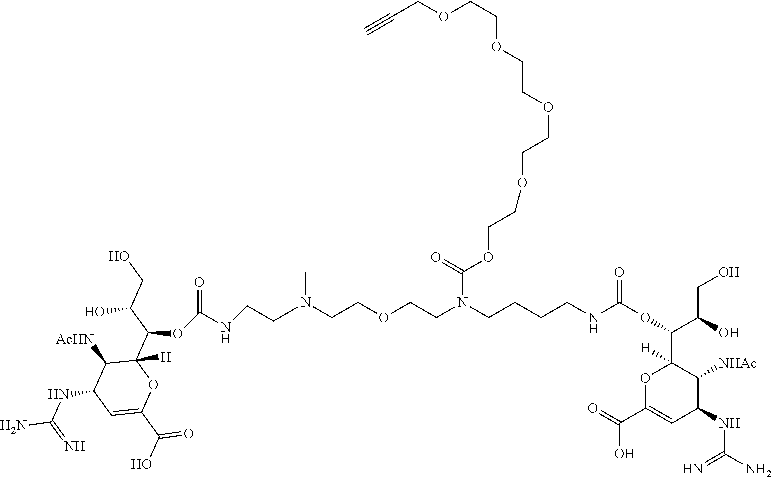 [SMILES: C#CCOCCOCCOCCOCCOC(=O)N(CCCCNC(=O)O[C@@H]([C@@H]1OC(C(=O)O)=C[C@H](NC(=N)N)[C@H]1NC(C)=O)[C@H](O)CO)CCOCCN(C)CCNC(=O)O[C@@H]([C@@H]1OC(C(=O)O)=C[C@H](NC(=N)N)[C@H]1NC(C)=O)[C@H](O)CO] |
| C-Inter-82 | 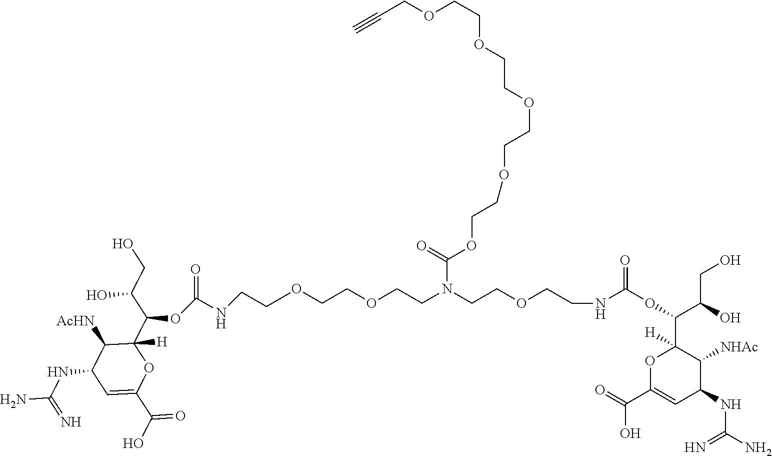 [SMILES: C#CCOCCOCCOCCOCCOC(=O)N(CCOCCNC(=O)O[C@@H]([C@@H]1OC(C(=O)O)=C[C@H](NC(=N)N)[C@H]1NC(C)=O)[C@H](O)CO)CCOCCOCCNC(=O)O[C@@H]([C@@H]1OC(C(=O)O)=C[C@H](NC(=N)N)[C@H]1NC(C)=O)[C@H](O)CO] |
| C-Inter-83 | 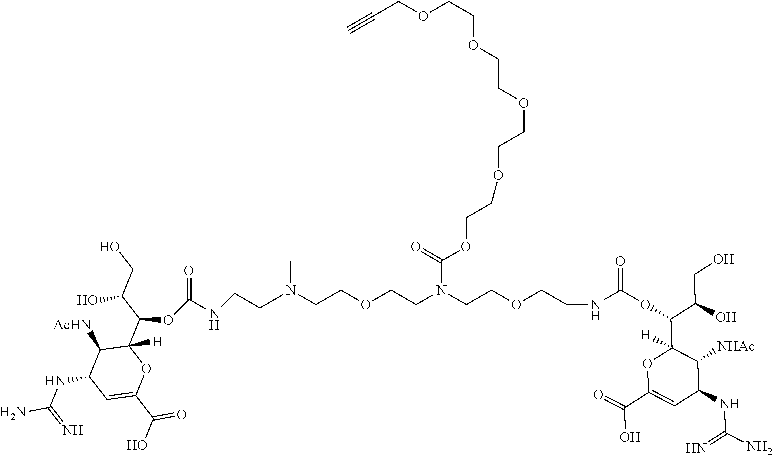 [SMILES: C#CCOCCOCCOCCOCCOC(=O)N(CCOCCNC(=O)O[C@@H]([C@@H]1OC(C(=O)O)=C[C@H](NC(=N)N)[C@H]1NC(C)=O)[C@H](O)CO)CCOCCN(C)CCNC(=O)O[C@@H]([C@@H]1OC(C(=O)O)=C[C@H](NC(=N)N)[C@H]1NC(C)=O)[C@H](O)CO] |
| C-Inter-84 | 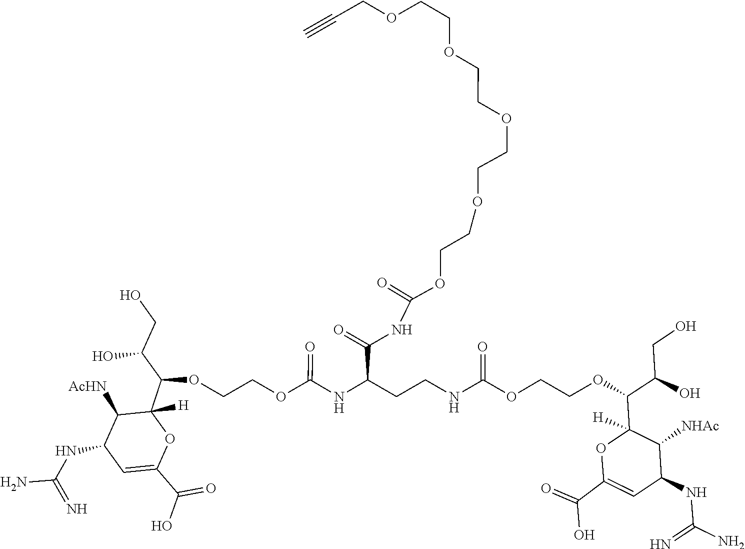 [SMILES: C#CCOCCOCCOCCOCCOC(=O)NC(=O)[C@@H](CCNC(=O)OCCO[C@@H]([C@@H]1OC(C(=O)O)=C[C@H](NC(=N)N)[C@H]1NC(C)=O)[C@H](O)CO)NC(=O)OCCO[C@@H]([C@@H]1OC(C(=O)O)=C[C@H](NC(=N)N)[C@H]1NC(C)=O)[C@H](O)CO] |
| C-Inter-85 | 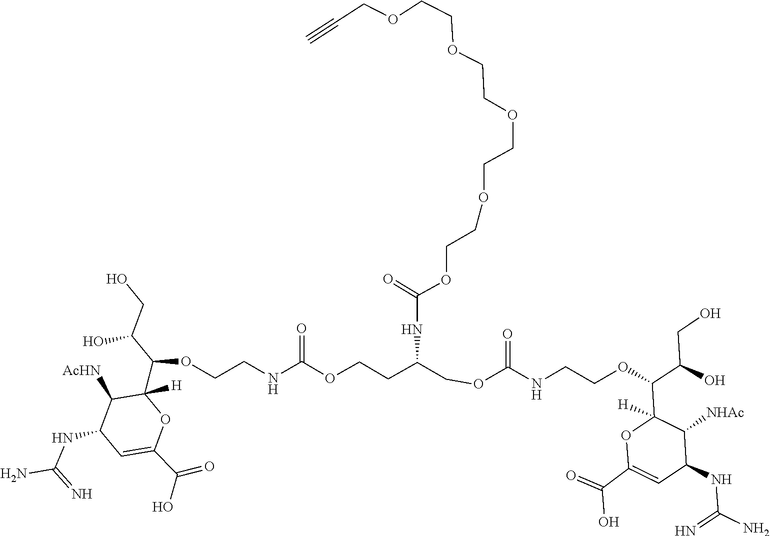 [SMILES: C#CCOCCOCCOCCOCCOC(=O)N[C@@H](CCOC(=O)NCCO[C@@H]([C@@H]1OC(C(=O)O)=C[C@H](NC(=N)N)[C@H]1NC(C)=O)[C@H](O)CO)COC(=O)NCCO[C@@H]([C@@H]1OC(C(=O)O)=C[C@H](NC(=N)N)[C@H]1NC(C)=O)[C@H](O)CO] |
| C-Inter-86 | 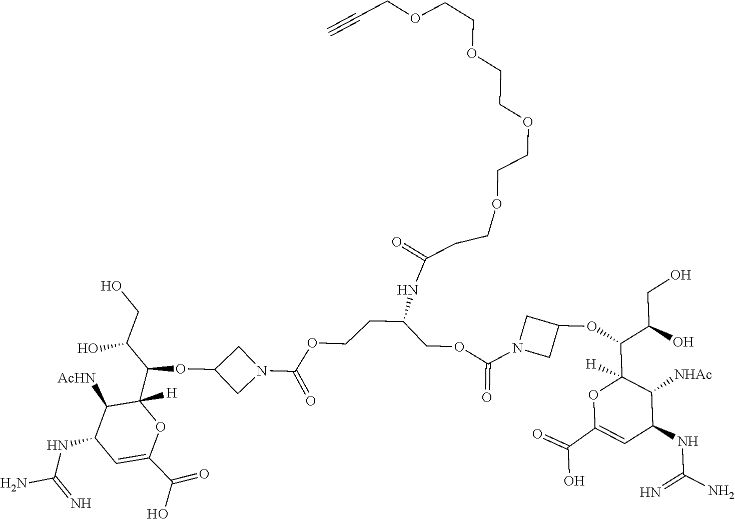 [SMILES: C#CCOCCOCCOCCOCCC(=O)N[C@@H](CCOC(=O)N1CC(O[C@@H]([C@@H]2OC(C(=O)O)=C[C@H](NC(=N)N)[C@H]2NC(C)=O)[C@H](O)CO)C1)COC(=O)N1CC(O[C@@H]([C@@H]2OC(C(=O)O)=C[C@H](NC(=N)N)[C@H]2NC(C)=O)[C@H](O)CO)C1] |
| C-Inter-87 | 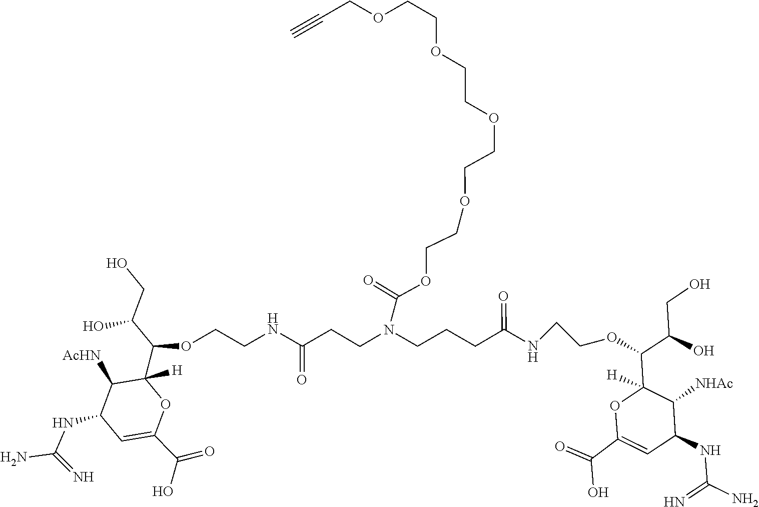 [SMILES: C#CCOCCOCCOCCOCCOC(=O)N(CCCC(=O)NCCO[C@@H]([C@@H]1OC(C(=O)O)=C[C@H](NC(=N)N)[C@H]1NC(C)=O)[C@H](O)CO)CCC(=O)NCCO[C@@H]([C@@H]1OC(C(=O)O)=C[C@H](NC(=N)N)[C@H]1NC(C)=O)[C@H](O)CO] |
| C-Inter-88 | 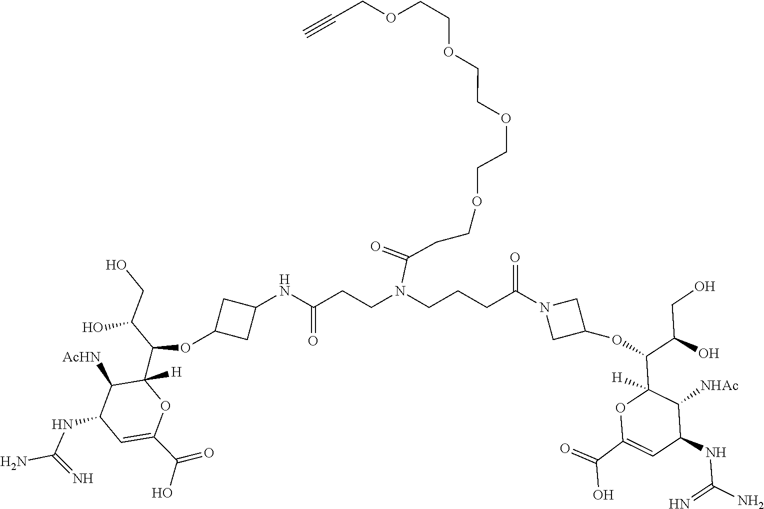 [SMILES: C#CCOCCOCCOCCOCCC(=O)N(CCCC(=O)N1CC(O[C@@H]([C@@H]2OC(C(=O)O)=C[C@H](NC(=N)N)[C@H]2NC(C)=O)[C@H](O)CO)C1)CCC(=O)NC1CC(O[C@@H]([C@@H]2OC(C(=O)O)=C[C@H](NC(=N)N)[C@H]2NC(C)=O)[C@H](O)CO)C1] |
| C-Inter-89 | 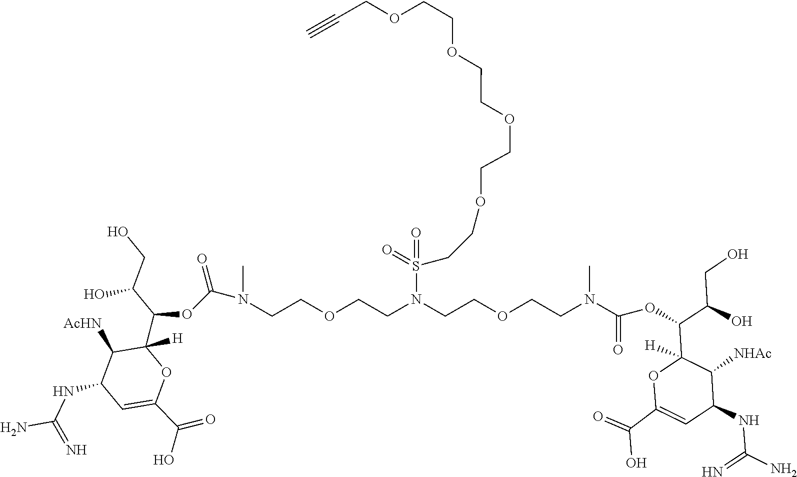 [SMILES: C#CCOCCOCCOCCOCCS(=O)(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-90 | 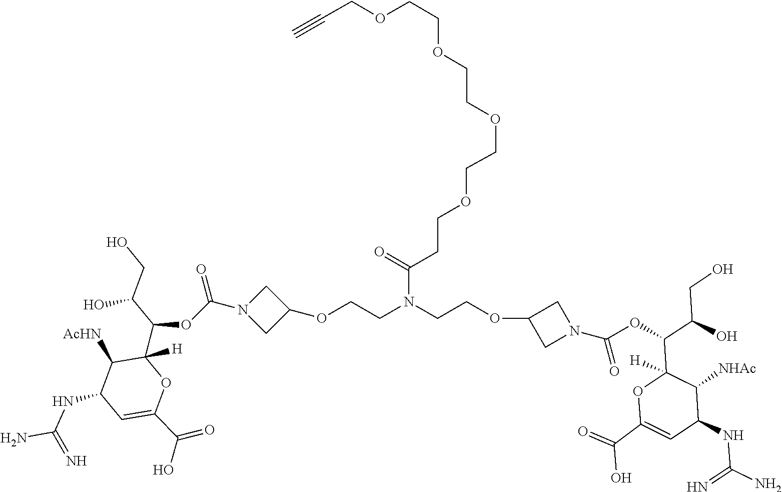 [SMILES: C#CCOCCOCCOCCOCCC(=O)N(CCOC1CN(C(=O)O[C@@H]([C@@H]2OC(C(=O)O)=C[C@H](NC(=N)N)[C@H]2NC(C)=O)[C@H](O)CO)C1)CCOC1CN(C(=O)O[C@@H]([C@@H]2OC(C(=O)O)=C[C@H](NC(=N)N)[C@H]2NC(C)=O)[C@H](O)CO)C1] |
| C-Inter-91 | 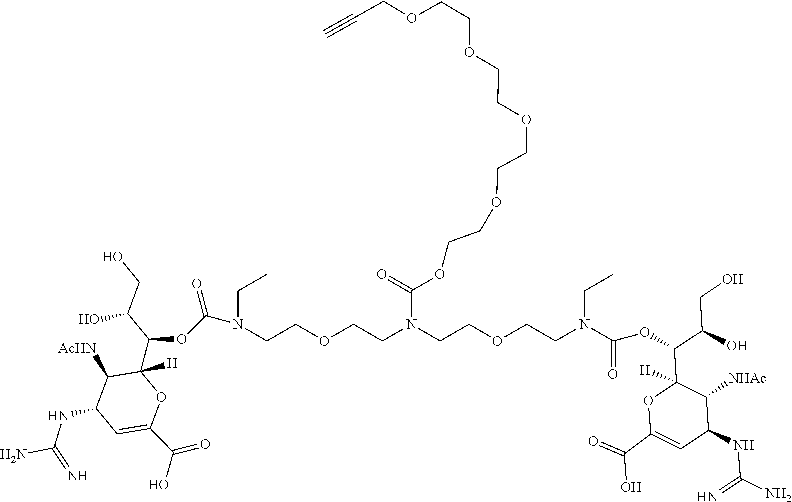 [SMILES: C#CCOCCOCCOCCOCCOC(=O)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C-Inter-92 | 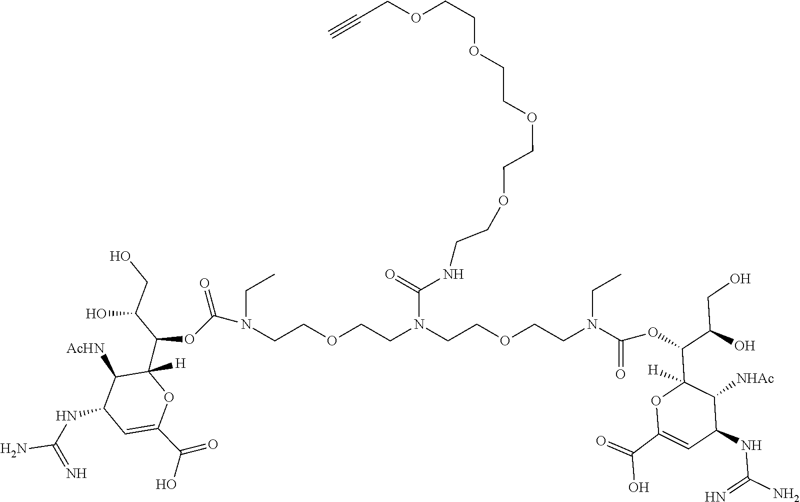 [SMILES: C#CCOCCOCCOCCOCCNC(=O)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C-Inter-93 | 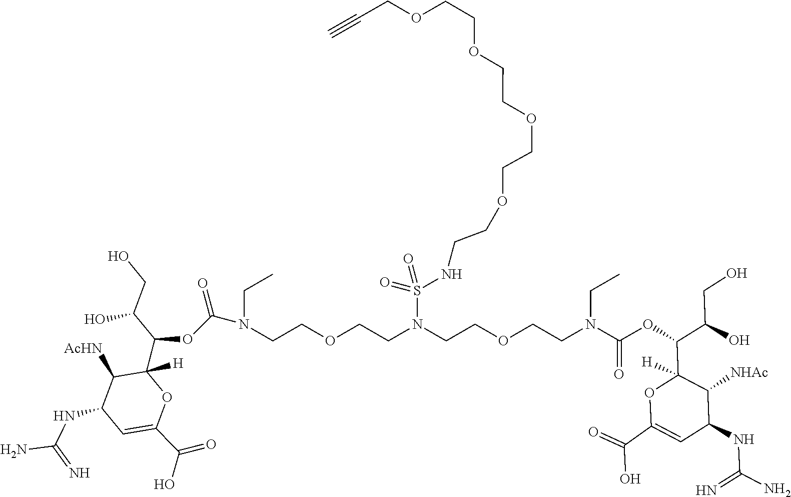 [SMILES: C#CCOCCOCCOCCOCCNS(=O)(=O)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C-Inter-94 | 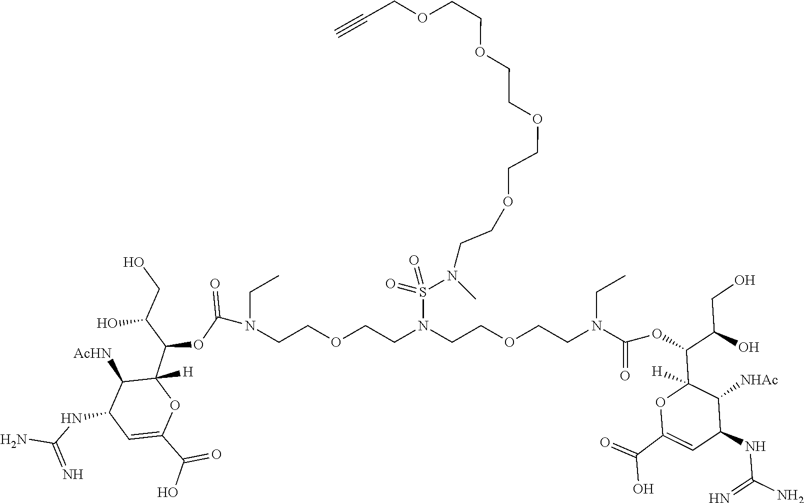 [SMILES: C#CCOCCOCCOCCOCCN(C)S(=O)(=O)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C-Inter-95 | 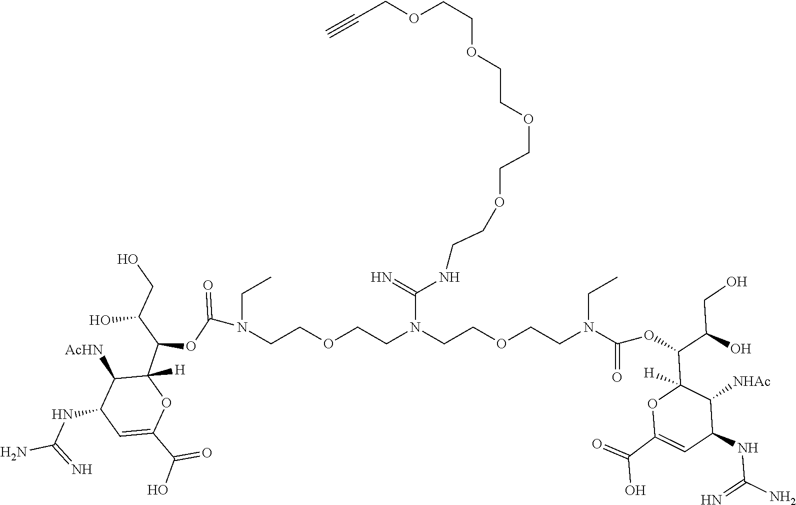 [SMILES: C#CCOCCOCCOCCOCCNC(=N)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C-Inter-96 | 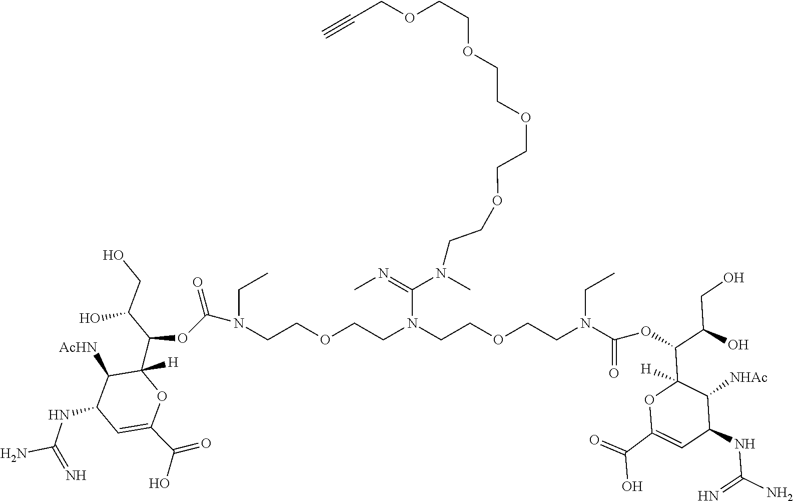 [SMILES: C#CCOCCOCCOCCOCCN(C)/C(=N/C)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| C-Inter-97 | 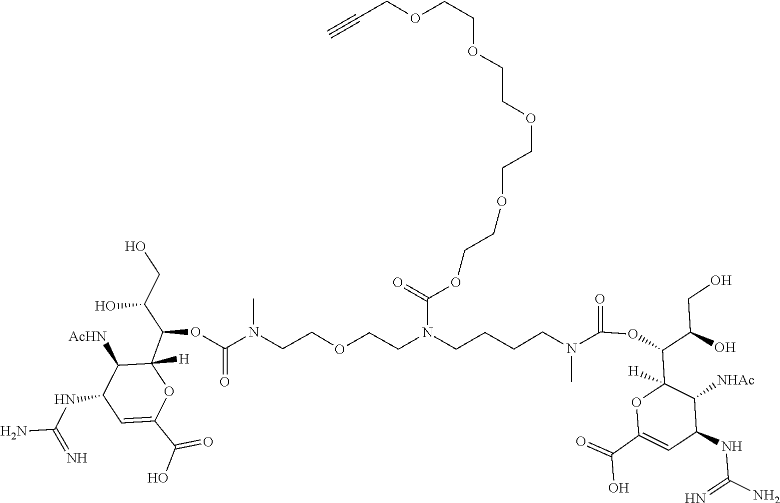 [SMILES: C#CCOCCOCCOCCOCCOC(=O)N(CCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-98 | 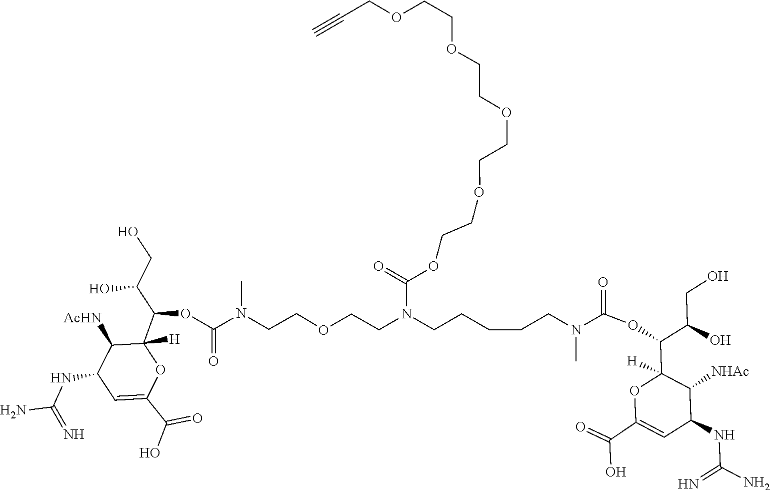 [SMILES: C#CCOCCOCCOCCOCCOC(=O)N(CCCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| C-Inter-99 | 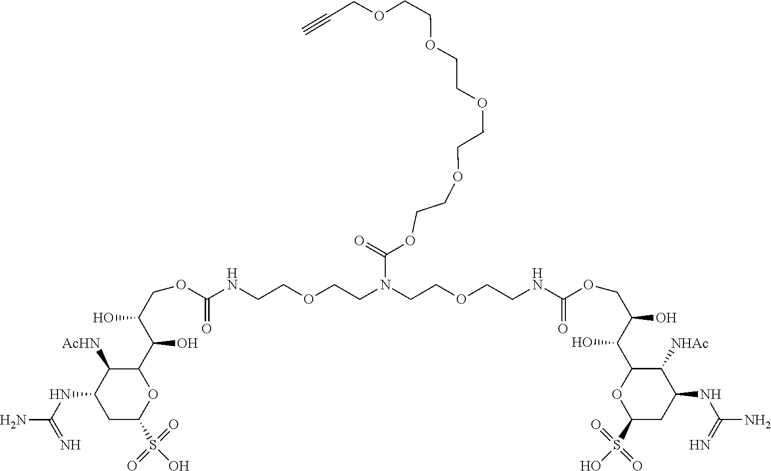 [SMILES: C#CCOCCOCCOCCOCCOC(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C-Inter-100 | 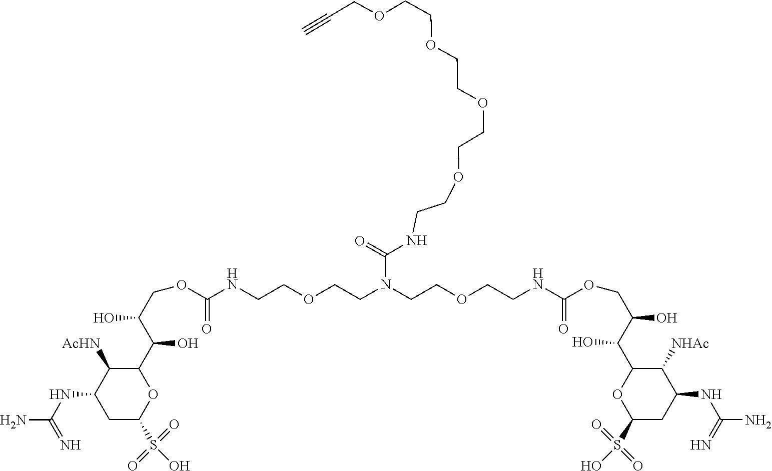 [SMILES: C#CCOCCOCCOCCOCCNC(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C-Inter-101 | 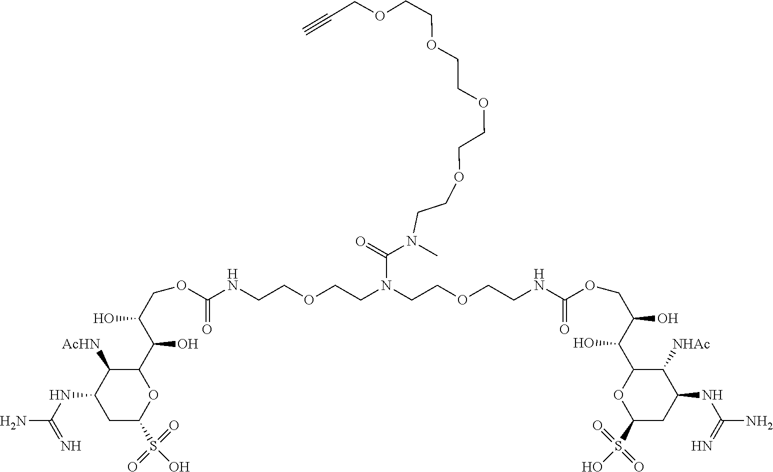 [SMILES: C#CCOCCOCCOCCOCCN(C)C(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C-Inter-102 | 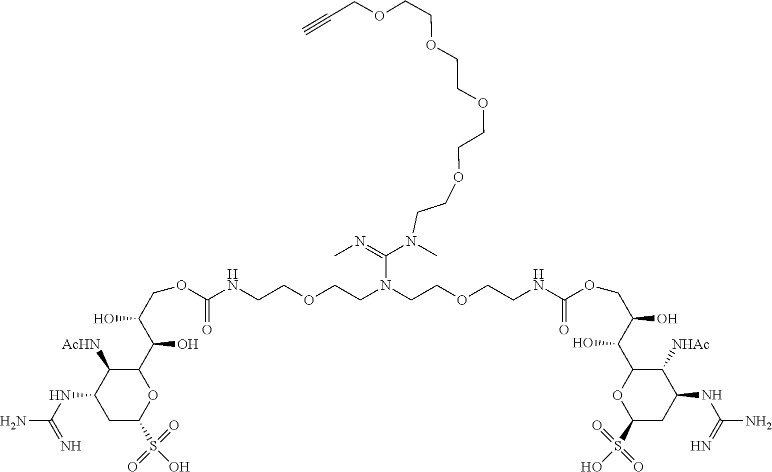 [SMILES: C#CCOCCOCCOCCOCCN(C)/C(=N/C)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C-Inter-103 | 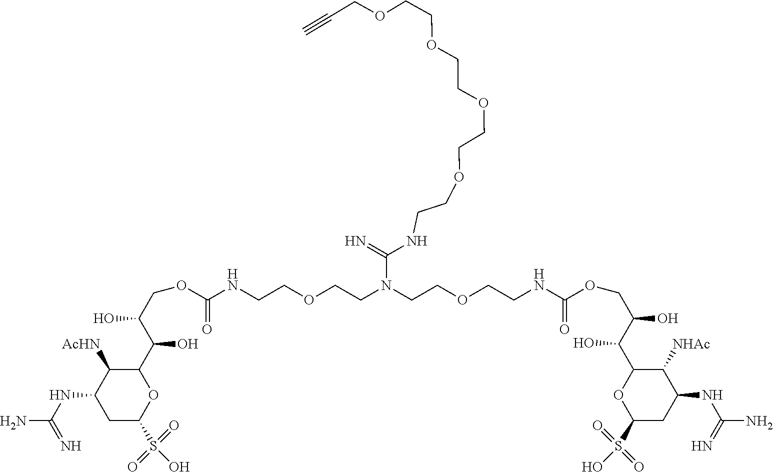 [SMILES: C#CCOCCOCCOCCOCCNC(=N)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C-Inter-104 | 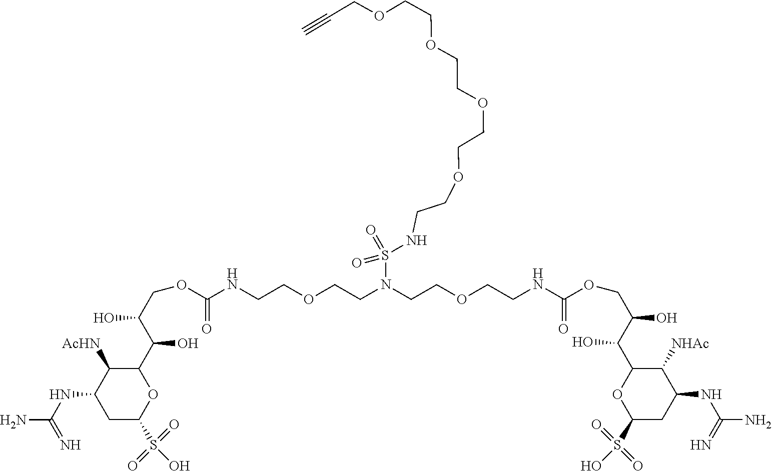 [SMILES: C#CCOCCOCCOCCOCCNS(=O)(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C-Inter-105 | 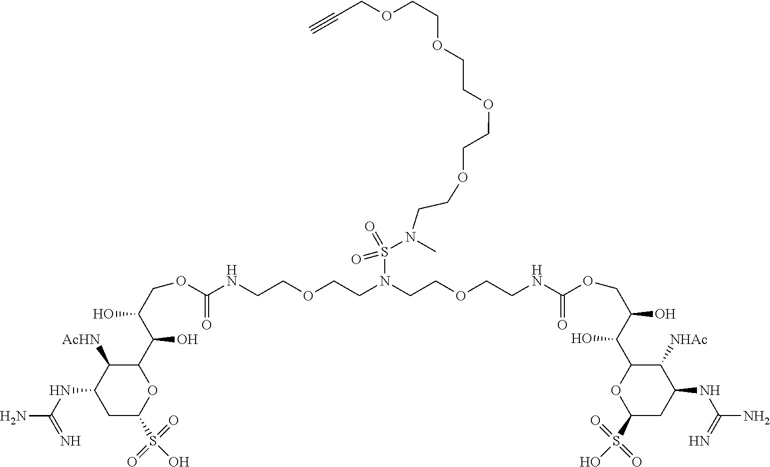 [SMILES: C#CCOCCOCCOCCOCCN(C)S(=O)(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C-Inter-106 | 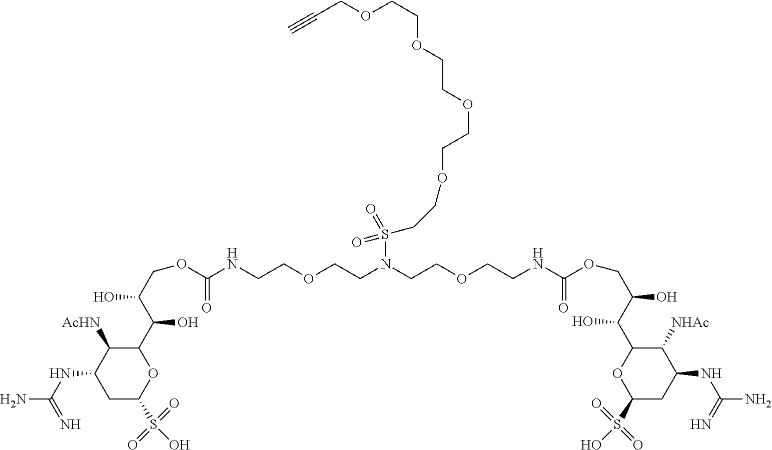 [SMILES: C#CCOCCOCCOCCOCCS(=O)(=O)N(CCOCCNC(=O)OC[C@@H](O)[C@@H](O)C1O[C@H](S(=O)(=O)O)C[C@H](NC(=N)N)[C@H]1NC(C)=O)CCOCCNC(=O)OC[C@@H](O)[C@@H](O)C1O[C@H](S(=O)(=O)O)C[C@H](NC(=N)N)[C@H]1NC(C)=O] |
| C-Inter-107 | 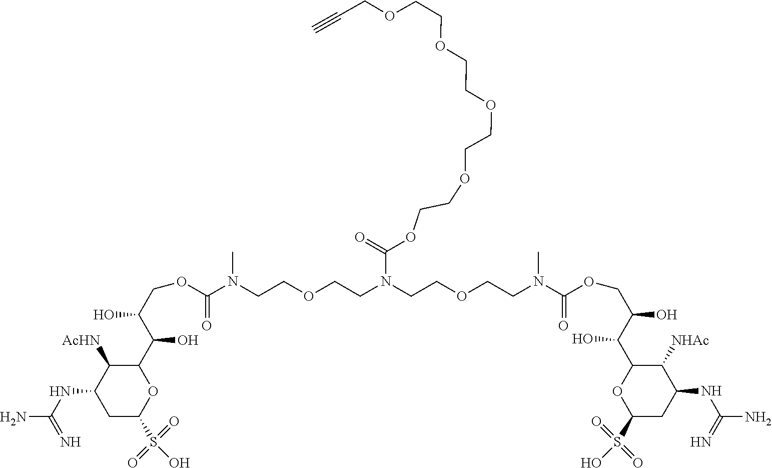 [SMILES: C#CCOCCOCCOCCOCCOC(=O)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C-Inter-108 | 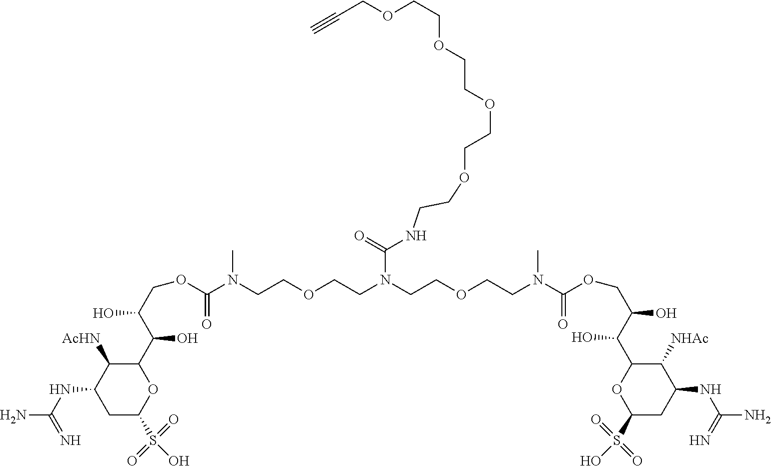 [SMILES: C#CCOCCOCCOCCOCCNC(=O)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C-Inter-109 | 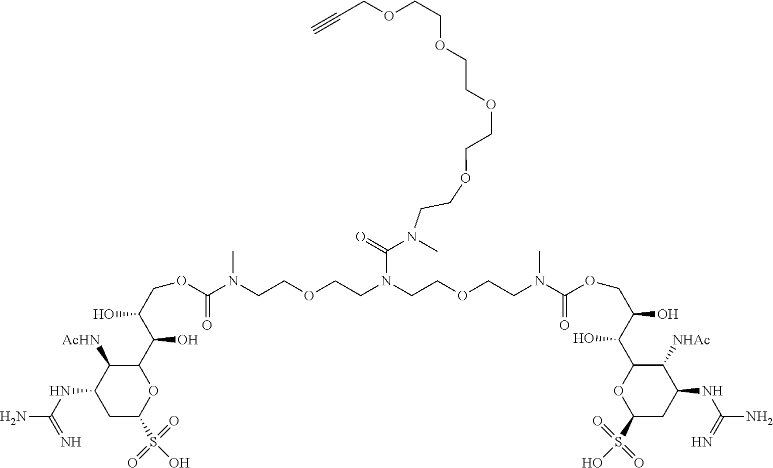 [SMILES: C#CCOCCOCCOCCOCCN(C)C(=O)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C-Inter-110 | 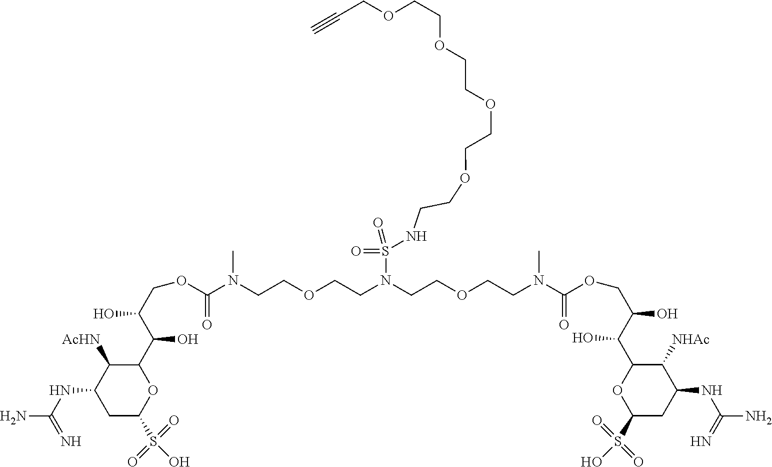 [SMILES: C#CCOCCOCCOCCOCCNS(=O)(=O)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C-Inter-111 | 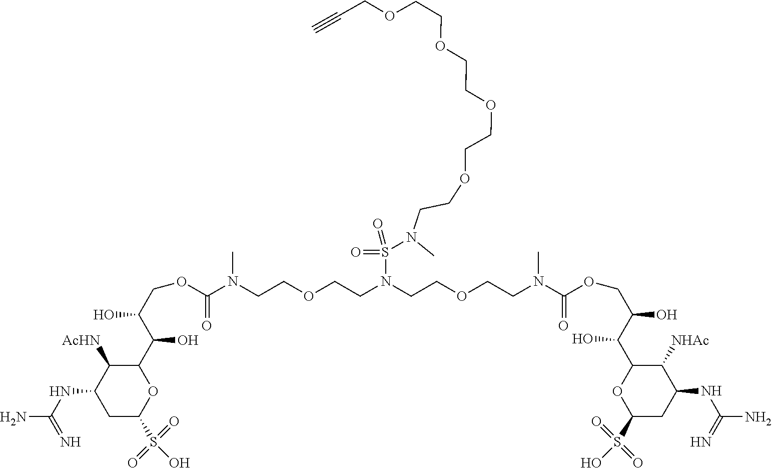 [SMILES: C#CCOCCOCCOCCOCCN(C)S(=O)(=O)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C-Inter-112 | 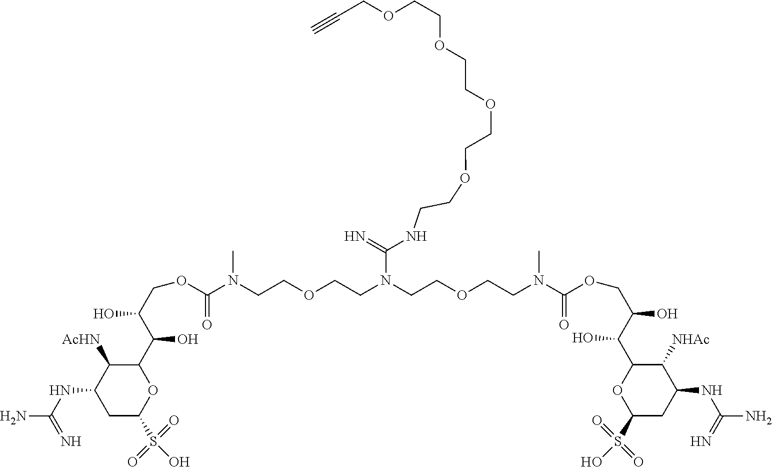 [SMILES: C#CCOCCOCCOCCOCCNC(=N)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C-Inter-113 | 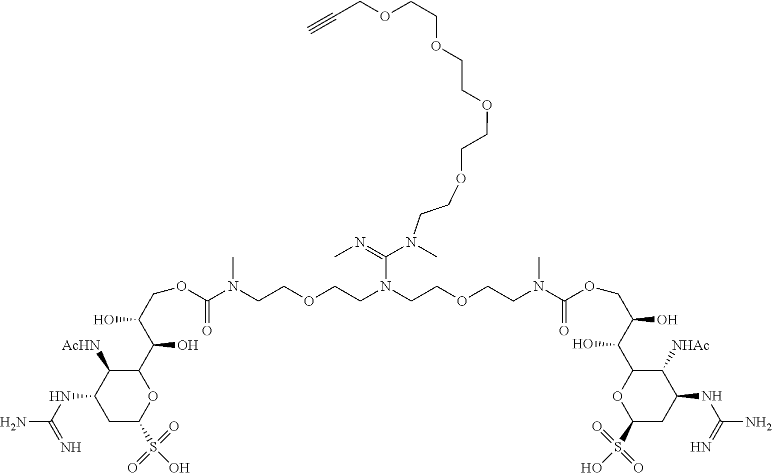 [SMILES: C#CCOCCOCCOCCOCCN(C)/C(=N/C)N(CCOCCN(C)C(=O)OC[C@@H](O)[C@@H](O)C1O[C@H](S(=O)(=O)O)C[C@H](NC(=N)N)[C@H]1NC(C)=O)CCOCCN(C)C(=O)OC[C@@H](O)[C@@H](O)C1O[C@H](S(=O)(=O)O)C[C@H](NC(=N)N)[C@H]1NC(C)=O] |
| C-Inter-114 | 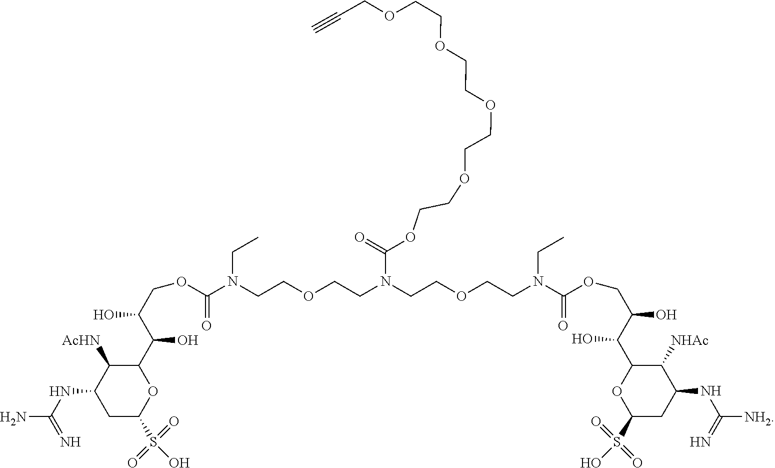 [SMILES: C#CCOCCOCCOCCOCCOC(=O)N(CCOCCN(CC)C(=O)OC[C@@H](O)[C@@H](O)C1O[C@H](S(=O)(=O)O)C[C@H](NC(=N)N)[C@H]1NC(C)=O)CCOCCN(CC)C(=O)OC[C@@H](O)[C@@H](O)C1O[C@H](S(=O)(=O)O)C[C@H](NC(=N)N)[C@H]1NC(C)=O] |

13. A Pharmaceutical composition comprising the conjugate of formula I-1 as claimed in claim 1, or a pharmaceutically acceptable salt, ester, isomer, solvate, prodrug or isotopically labeled derivative thereof, and optionally one or more other therapeutic agents, such as chemotherapeutic agents, angiogenesis inhibitors, cytokines, cytotoxic agents, other antibodies, other small molecule drugs or immunomodulators (e.g., immune checkpoint inhibitors or agonists), and optionally pharmaceutically acceptable excipients.

14. A method for the prevention or treatment of a subject having a viral infection or at a risk of having a viral infection, comprising administering to the subject, for example by injection, an effective amount of the conjugate of formula I-1 as claimed in claim 1, a pharmaceutically acceptable salt, ester, isomer, solvate, prodrug or isotopically labeled derivative thereof.

15. The method of claim 14, wherein
the viral infection is an infection caused by an influenza virus or a parainfluenza virus;
preferably, the viral infection is an infection caused by influenza virus A, B or C, or parainfluenza virus;
preferably, the subject having a viral infection or at a risk of having a viral infection may be a subject with an immune system deficiency;
preferably, the subject having a viral infection or at a risk of having a viral infection may be a subject who is or will be treated with an immunosuppressive agent, preferably, the subject having a viral infection or at a risk of having a viral infection may be a subject diagnosed with an immunosuppression disease;
preferably, the subject diagnosed with an immunosuppression disease has cancer or acquired immunodeficiency syndrome;
preferably, the subject diagnosed with an immunosuppression disease has leukemia, lymphoma, humoral immunodeficiency, T-cell deficiency, complement deficiency or multiple myeloma;
preferably, the subject is a subject undergoing or about to undergo a hematopoietic stem cell transplant;
preferably, the subject is a subject undergoing or about to undergo an organopoietic transplant; and/or
preferably, the subject may be at a risk of a secondary infection.

16. A method for preventing a secondary infection of a subject caused by an influenza virus infection, comprising administering to the subject, for example by an injection, an effective amount of the conjugate of formula I-1 as claimed in claim 1, a pharmaceutically acceptable salt, ester, isomer, solvate, prodrug or isotopically labeled derivative thereof,
preferably, said secondary infection is a respiratory infection;
preferably, said secondary infection is associated with pneumonia;
preferably, said secondary infection is a bacterial, or viral, or fungal infection;
preferably, said bacterial infection is an infection caused by methicillin-resistant Staphylococcus aureus; preferably, said bacterial infection is an infection caused by Streptococcus pneumoniae;
preferably, the conjugate of formula I-1 or the compound of formula I-2, or pharmaceutically acceptable salt, ester, isomer, solvate, prodrug or isotopically labeled derivative thereof, is administrated by an intramuscular injection, intravenous injection, intradermal injection, intra-arterial injection, intraperitoneal injection, intra-lesional injection, intracranial injection, intra-articular injection, intrapleural injection, intratracheal injection, intraprostatic injection, intranasal injection, intravitreous injection, intravaginal injection, intrarectal injection, local injection, intra-tumor injection, intraperitoneal injection, subcutaneous injection, subconjunctival injection, intracapsular injection, mucosal injection, intrapericardial injection, intra-umbilical injection, intra-ocular injection, oral, local inhalation, injection or infusion,
preferably, the conjugate of formula I-1 or the compound of formula I-2, or pharmaceutically acceptable salt, ester, isomer, solvate, prodrug or isotopically labeled derivative thereof, combines with another therapeutic agent in administration or in the preparation of a medicament;
preferably, the another therapeutic agent is an antiviral drug;
preferably, the antiviral drug is baloxavir, pimodivir, oseltamivir, zanamivir, peramivir, laninamivir, amantadine, MEDI8852 or rimantadine;
preferably, another drug used by the subject is an antiviral vaccine;
preferably, the antiviral drug and the conjugate of formula I-1 or the compound of formula I-2, or pharmaceutically acceptable salt, ester, isomer, solvate, prodrug or isotopically labeled derivative thereof, are sequentially administered, for example, by injection to the subject; and/or
preferably, the antiviral drug and the conjugate of formula I-1 or the compound of formula I-2, or pharmaceutically acceptable salt, ester, isomer, solvate, prodrug or isotopically labeled derivative thereof, are simultaneously administered, for example by injection, to the subject.


SEQUENCE LISTING (71 sequences)

SEQ ID NO: 1 - protein, 252 aa, synthetic construct
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 2 - protein, 232 aa, synthetic construct
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 251 aa, synthetic construct
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 231 aa, synthetic construct
MVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 258 aa, synthetic construct
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 6 - protein, 238 aa, synthetic construct
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 7 - protein, 257 aa, synthetic construct
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 8 - protein, 237 aa, synthetic construct
MVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 9 - protein, 262 aa, synthetic construct
MYRMQLLSCIALSLALVTNSMVRSDKTHTCPPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 10 - protein, 242 aa, synthetic construct
MVRSDKTHTCPPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 11 - protein, 237 aa, synthetic construct
MVRSDKTHTCPPCPPCKCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQMQGNVFSCSVMHEALHNHYTQKSLSLSPGK

SEQ ID NO: 12 - protein, 258 aa, synthetic construct
MYRMQLLSCIALSLALVTNSPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 13 - protein, 238 aa, synthetic construct
PRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKHHHHHH

SEQ ID NO: 14 - protein, 257 aa, synthetic construct
MYRMQLLSCIALSLALVTNSMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 15 - protein, 237 aa, synthetic construct
MVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 16 - protein, 252 aa, synthetic construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 266 aa, synthetic construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 18 - protein, 248 aa, synthetic construct
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 19 - protein, 252 aa, synthetic construct
MKWVTFISLLFLFSSAYSISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 234 aa, synthetic construct
ISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 266 aa, synthetic construct
MKWVTFISLLFLFSSAYSISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 22 - protein, 248 aa, synthetic construct
ISAMVRSDKTHTCPPCPAPELLGGPSVFLFPPKPSDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 23 - protein, 266 aa, synthetic construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 24 - protein, 248 aa, synthetic construct
ISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 25 - protein, 266 aa, synthetic construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGGGGGSEQKLISEEDL

SEQ ID NO: 26 - protein, 248 aa, synthetic construct
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGGGGGSEQKLISEEDL

SEQ ID NO: 27 - protein, 266 aa, synthetic construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 28 - protein, 248 aa, synthetic construct
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 29 - protein, 266 aa, synthetic construct
MKWVTFISLLFLFSSAYSISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 30 - protein, 248 aa, synthetic construct
ISAMVRSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 31 - protein, 259 aa, synthetic construct
MKWVTFISLLFLFSSAYSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQFLISEEDL

SEQ ID NO: 32 - protein, 245 aa, synthetic construct
MKWVTFISLLFLFSSAYSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 231 aa, synthetic construct
MVRSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 250 aa, synthetic construct
MKWVTFISLLFLFSSAYSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 245 aa, synthetic construct
MGWSCIILFLVATATGVHSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 226 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 226 aa, synthetic construct
KTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 38 - protein, 259 aa, synthetic construct
MKWVTFISLLFLFSSAYSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 39 - protein, 226 aa, synthetic construct
DKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 40 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 45 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 46 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 47 - protein, 265 aa, synthetic construct
MGWSCIILFLVATATGVHSNVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 48 - protein, 266 aa, synthetic construct
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 266 aa, synthetic construct
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 266 aa, synthetic construct
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 51 - protein, 266 aa, synthetic construct
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 52 - protein, 266 aa, synthetic construct
MGWSCIILFLVATATGVHSNVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 266 aa, synthetic construct
MGWSCIILFLVATATGVHSNVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 267 aa, synthetic construct
MGWSCIILFLVATATGVHSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSEQKLISEEDL

SEQ ID NO: 55 - protein, 267 aa, synthetic construct
MGWSCIILFLVATATGVHSISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSEQKLISEEDL

SEQ ID NO: 56 - protein, 267 aa, synthetic construct
MGWSCIILFLVATATGVHSISAMVRSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 57 - protein, 267 aa, synthetic construct
MGWSCIILFLVATATGVHSISAMVRSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 58 - protein, 275 aa, synthetic construct
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSQRNPRLRLIRRHPTLRIPPI

SEQ ID NO: 59 - protein, 275 aa, synthetic construct
MGWSCIILFLVATATGVHSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSQRNPRLRLIRRHPTLRIPPI

SEQ ID NO: 60 - protein, 247 aa, synthetic construct
NVNHKPSNTKVDKKVEPKSZDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 61 - protein, 247 aa, synthetic construct
NVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 62 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 66 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 67 - protein, 247 aa, synthetic construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 247 aa, synthetic construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 20 aa, synthetic construct
MYRMQLLSCIALSLALVTNS

SEQ ID NO: 70 - protein, 18 aa, synthetic construct
MKWVTFISLLFLFSSAYS

SEQ ID NO: 71 - protein, 19 aa, synthetic construct
MGWSCIILFLVATATGVHS